(19) United States
(12) Patent Application Publication
(10) Pub. No.: US 2026/0327651 A1
(43) Pub. Date: Oct. 22, 2026

(54) LENTIVIRAL VECTORS WITH TWO OR MORE GENOMES

(71) Applicant: Sana Biotechnology, Inc., Seattle, WA (US)

(72) Inventors: Diana VEDENOVA, Seattle, WA (US); Ryan WHITE, Seattle, WA (US)

(21) Appl. No.: 19/476,262

(22) PCT Filed: Apr. 18, 2024
(86) PCT No.: PCT/US2024/025081

§ 371 Date: Oct. 17, 2025

Related U.S. Application Data
(60) Provisional application No. 63/496,933, filed on Apr. 18, 2023.
(60) Provisional application No. 63/517,054, filed on Aug. 1, 2023.

(51) Int. Cl. C12N 15/86; A61K 40/11; A61K 40/31; A61K 40/42; A61P 35/00; C07K 14/005; C07K 14/705; C07K 16/28; C12N 5/0783; C12N 9/22; C12N 15/11

(52) U.S. Cl. CPC C12N 15/86; A61K 40/11; A61K 40/31; A61K 40/4202; A61K 40/4211; A61K 40/4212; A61K 40/4221; A61P 35/00; C07K 14/005; C07K 14/70503; C07K 16/28; C07K 16/2803; C07K 16/2809; C07K 16/2878; C12N 5/0636; C12N 9/226; C12N 15/11; C12N 2310/20; C12N 2510/00; C12N 2740/15043; C12N 2740/15052; C12N 2760/18222

(57) ABSTRACT

Among other things, provided are an engineered lentiviral vector comprising a first genome and a second genome. A first genome can comprise a first transgene. A second genome can comprise a second transgene. In some embodiments, a first and a second transgene are different. Further provided are engineered lentiviral vector systems for producing such an engineered lentiviral vector. Also, provided are methods of making engineered lentiviral vectors and compositions comprising engineered lentiviral vectors.

### CROSS-REFERENCE TO RELATED APPLICATIONS

The present application claims priority from U.S. Provisional Application No. 63/496,933 filed on Apr. 18, 2023 and U.S. Provisional Application No. 63/517,054 filed on Aug. 1, 2023 the contents of each of which are incorporated by reference in their entirety.

### BACKGROUND

Viral vectors provide an efficient means for modification of eukaryotic cells. Accordingly, the use of viral vectors continues to be important for both research and clinical gene therapy applications. One viral vector system that has been developed is the lentiviral vector system, which has been derived from the human immunodeficiency virus.

### SUMMARY OF THE INVENTION

To achieve expression of two different transgenes in cell sample using a viral vector (e.g., a lentiviral vector), sequential and/or simultaneous transduction of the cell sample (e.g., in vitro, ex vivo, or in vivo cells) would be required. These methods can be complicated from a technical perspective because such methods involve utilizing two different lentiviral vector preparations in a specified way, and managing differing analytical profiles and experimental timepoints. These methods can be also complicated from a biological perspective because of potential restriction factors in play). Moreover, the methods can be complicated from a manufacturing/clinical perspective because two separate lentiviral vector batches would be necessary, and would lead to additional regulatory assay, reagent, and product testing.

The present disclosure provides, among other things, an engineered lentiviral vector comprising a plurality (e.g., two or more) of genomes. An engineered lentiviral vector comprising a plurality of genomes overcomes the challenges associated with sequential and/or simultaneous transduction because rather than, e.g., producing multiple lentiviral vector compositions and performing multiple transductions, a single lentiviral composition can be produced that needs only a single transduction to deliver a plurality of transgenes. The present disclosure also provides lentiviral vector systems, e.g., for the production of lentiviral vectors, as well as methods of using the same.

Among other things, the present disclosure provides an engineered lentiviral vector comprising a first genome and a second genome. In some embodiments, a first genome comprises a first transgene. In some embodiments, a second genome comprises a second transgene. In some embodiments, a first transgene and a second transgene are different.

In some embodiments, a first transgene and/or a second transgene encodes a nuclease. In some embodiments, a nuclease is a Cas, a TALEN, or a zinc-finger nuclease.

In some embodiments, a first transgene and/or a second transgene encodes a gRNA.

In some embodiments, a first transgene and/or a second transgene encodes an antibody or portion thereof.

In some embodiments, a first transgene and/or a second transgene encodes a chimeric antigen receptor. In some embodiments, a CAR is a CD19 CAR, CD20 CAR, CD22 CAR, or BCMA CAR.

In some embodiments, a first transgene and/or a second transgene encodes an antigen.

In some embodiments, a first transgene and/or a second transgene encodes a therapeutic polypeptide. In some embodiments, a therapeutic polypeptide is useful for protein replacement therapy.

In some embodiments, a first transgene encodes a gRNA and a second transgene encodes a Cas nuclease.

In some embodiments, a first transgene encodes a CD19 CAR and a second transgene encodes a CD22 CAR.

In some embodiments, a first genome and/or second genome further comprises a transgene encoding a tolerogenic factor. In some embodiments, a tolerogenic factor is CD47, DUX4, CD24, CD27, CD35, CD46, CD55, CD59, CD200, HLA-C, HLA-E, HLA-E heavy chain, HLA-G, PD-L1, IDO1, CTLA4-Ig, C1-Inhibitor, IL-10, IL-35, FasL, CCL21, CCL22, Mfge8, CD16, CD52, H2-M3, CD16 Fc receptor, IL15-RF, and Serpinb9, A20/TNFAIP3, CD39, CR1, HLA-F, IL15-RF, or MANF. In some embodiments, a tolerogenic factor is CD47. In some embodiments, a first genome further comprises a transgene encoding CD47 and a second genome further comprises a transgene encoding CD47.

In some embodiments, an engineered lentiviral vector comprises a bilayer envelope that comprises one or more fusogens.

In some embodiments, one or more fusogens comprise at least one fusogen that has a tropism for B cells, T cells, natural killer cells, islet cells, glial progenitor cells, neuronal cells, hematopoietic stem cells, cardiac cells, hepatocytes, stem cells, or induced pluripotent stem cells.

In some embodiments, one or more fusogens comprise at least one fusogen that has an endogenous tropism. In some embodiments, one or more fusogens comprise at least one fusogen that has engineered tropism.

In some embodiments, one or more fusogens comprise one or more viral fusogens. In some embodiments, one or more fusogens comprise at least one fusogen that is involved in attachment of a viral vector to a cell membrane. In some embodiments, one or more fusogens comprise at least one fusogen that is involved in directing fusion of the lipid bilayer of a viral vector and a cell membrane.

In some embodiments, one or more fusogens comprise one or more paramyxovirus envelope proteins or biologically active portions thereof. In some embodiments, one or more paramyxovirus envelope proteins or biologically active portions thereof comprises a paramyxovirus glycoprotein (“Protein G”) or a biologically active portion thereof. In some embodiments, one or more paramyxovirus envelope proteins or biologically active portions thereof comprises a paramyxovirus fusion protein (“Protein F”) or a biologically active portion thereof.

In some embodiments, one or more fusogens comprise one or more chimeric proteins. In some embodiments, one or more chimeric proteins comprise at least one chimeric protein that comprises a paramyxovirus envelope protein or biologically active portion thereof. In some embodiments, one or more chimeric proteins comprise at least one chimeric protein that comprises an scFV. In some embodiments, one or more chimeric proteins comprise at least one chimeric protein that comprises (i) a paramyxovirus envelope protein or biologically active portion thereof and (ii) an scFV.

In some embodiments, an scFV targets an antigen present on the surface of a B cell, a T cell, a natural killer cell, an islet cell, a glial progenitor cell, a cardiac cell, a blood cell, a hepatocyte, a stem cell, or an induced pluripotent stem cell. In some embodiments, an scFV targets an antigen present on the surface of a B cell. In some embodiments, an scFV targets an antigen present on the surface of a T cell. In some embodiments, an scFV targets CD8. In some embodiments, an scFV targets CD4. In some embodiments, an scFV targets an antigen present on the surface of an islet cell. In some embodiments, an islet cell is an alpha cell, a beta cell, or a delta cell. In some embodiments, an islet cell is a beta cell.

In some embodiments, a first genome and/or second genome further comprises one or more of: a promoter, an RSV promoter, a T7 promoter, a UbC promoter, a 5′ LTR, a truncated HIV-1 5′ LTR, an HIV-1 Ψ packaging signal, a RRE sequence, HIV-1 cPPT/CTS sequence, a P2A sequence, a T2A sequence, a WPRE sequence, a 3′ LTR, a self-inactivating HIV-1 3′ LTR, a poly A sequence, an origin of replication (ori), SV40 ori, and a reporter gene.

Also provided herein is an engineered lentiviral vector system comprising one or more envelope plasmids, a packaging plasmid, and two or more transfer plasmids. In some embodiments, two or more transfer plasmids comprise a first transfer plasmid that comprises a first transgene and a second transfer plasmid that comprises a second transgene. In some embodiments, a first and a second transgene are different.

In some embodiments, one or more envelope plasmids encode one or more fusogens.

In some embodiments, one or more fusogens comprise at least one fusogen that has a tropism for B cells, T cells, natural killer cells, islet cells, glial progenitor cells, neuronal cells, hematopoietic stem cells, cardiac cells, hepatocytes, stem cells, or induced pluripotent stem cells.

In some embodiments, one or more fusogens comprise at least one fusogen that has an endogenous tropism. In some embodiments, one or more fusogens comprise at least one fusogen that has engineered tropism.

In some embodiments, one or more fusogens comprise one or more viral fusogens. In some embodiments, one or more fusogens comprise at least one fusogen that is involved in attachment of a viral vector to a cell membrane. In some embodiments, one or more fusogens comprise at least one fusogen that is involved in directing fusion of the lipid bilayer of a viral vector and a cell membrane.

In some embodiments, one or more fusogens comprise one or more paramyxovirus envelope proteins or biologically active portions thereof. In some embodiments, one or more paramyxovirus envelope proteins or biologically active portions thereof comprises a paramyxovirus glycoprotein (“Protein G”) or portion thereof. In some embodiments, one or more paramyxovirus envelope proteins or biologically active portions thereof comprises a paramyxovirus fusion protein (“Protein F”) or portion thereof.

In some embodiments, one or more fusogens comprise one or more chimeric proteins. In some embodiments, one or more chimeric proteins comprise at least one chimeric protein that comprises a paramyxovirus envelope protein or biologically active portion thereof. In some embodiments, one or more chimeric proteins comprise at least one chimeric protein that comprises an scFV. In some embodiments, one or more chimeric proteins comprise at least one chimeric protein that comprises (i) a paramyxovirus envelope protein or biologically active portion thereof and (ii) an scFV.

In some embodiments, an scFV targets an antigen present on the surface of a B cell, a T cell, a natural killer cell, an islet cell, a glial progenitor cell, a cardiac cell, a blood cell, a hepatocyte, a stem cell, or an induced pluripotent stem cell. In some embodiments, an scFV targets an antigen present on the surface of a B cell. In some embodiments, an scFV targets an antigen present on the surface of a T cell. In some embodiments, an scFV targets CD8. In some embodiments, an scFV targets CD4. In some embodiments, an scFV targets an antigen present on the surface of an islet cell. In some embodiments, an islet cell is an alpha cell, a beta cell, or a delta cell. In some embodiments, an islet cell is a beta cell.

In some embodiments, a first transgene and/or a second transgene encodes a nuclease. In some embodiments, a nuclease is a Cas, a TALEN, or a zinc-finger nuclease.

In some embodiments, a first transgene and/or a second transgene encodes a gRNA.

In some embodiments, a first transgene and/or a second transgene encodes an antibody or portion thereof.

In some embodiments, a first transgene and/or a second transgene encodes a chimeric antigen receptor. In some embodiments, a CAR is a CD19 CAR, CD20 CAR, CD22 CAR, or BCMA CAR.

In some embodiments, a first transgene and/or a second transgene encodes an antigen.

In some embodiments, a first transgene and/or a second transgene encodes a therapeutic polypeptide. In some embodiments, a therapeutic polypeptide is useful for protein replacement therapy.

In some embodiments, a first transgene encodes a gRNA and a second transgene encodes a Cas nuclease.

In some embodiments, a first transgene encodes a CD19 CAR and a second transgene encodes a CD22 CAR.

In some embodiments, a first genome and/or second genome further comprises a transgene encoding a tolerogenic factor. In some embodiments, a tolerogenic factor is CD47, DUX4, CD24, CD27, CD35, CD46, CD55, CD59, CD200, HLA-C, HLA-E, HLA-E heavy chain, HLA-G, PD-L1, IDO1, CTLA4-Ig, C1-Inhibitor, IL-10, IL-35, FasL, CCL21, CCL22, Mfge8, CD16, CD52, H2-M3, CD16 Fc receptor, IL15-RF, and Serpinb9, A20/TNFAIP3, CD39, CR1, HLA-F, IL15-RF, or MANF. In some embodiments, a tolerogenic factor is CD47. In some embodiments, a first genome further comprises a transgene encoding CD47 and a second genome further comprises a transgene encoding CD47.

The present disclosure further provides a method of making lentiviral vectors. In some embodiments, a method of making lentiviral vectors comprises introducing an engineered lentiviral vector system as described herein to producer cells. In some embodiments, a method of making lentiviral vectors comprises culturing the producer cells under conditions sufficient to produce the viral vectors.

In some embodiments, the present disclosure provides an engineered lentiviral vector comprising a first genome comprising a first transgene; and a second genome comprising a second transgene, wherein the first transgene encodes a first chimeric antigen receptor (CAR) and the second transgene encodes a second CAR, wherein the first CAR and the second CAR comprise different antigen binding domains that target different antigens expressed on a target cell.

In some embodiments, a first CAR is a CD19 CAR and a second CAR is a CD22 CAR; a first CAR is a B cell maturation agent (BCMA) CAR and a second CAR is a G-protein coupled receptor family C group 5 member D (GPRC5D) CAR; a first CAR is a CD19 CAR and a second CAR is a CD20 CAR; a first CAR is a CD20 CAR and a second CAR is a CD22 CAR, or a first CAR is a CD19 CAR and a second CAR is a BCMA CAR.

In some embodiments, a first genome and/or a second genome further comprises a transgene encoding a tolerogenic factor.

Further provided herein is a method of making a drug substance comprising a lentiviral vector. In some embodiments, a method of making a drug substance comprises making lentiviral vectors as described herein. In some embodiments, a method of making a drug substance comprises obtaining a subset of the producer cell culture comprising lentiviral vectors. In some embodiments, a method of making a drug substance comprises enriching lentiviral vectors from the subset of the producer cell culture. In some embodiments, a method of making a drug substance comprises removing producer cell DNA and/or producer cell protein from the subset of the producer cell culture. In some embodiments, a method of making a drug substance comprises adding a pharmaceutically acceptable excipient to the enriched lentiviral vectors.

### BRIEF DESCRIPTION OF THE DRAWING

The Drawing included herein, which is composed of the following Figures, is for illustration purposes only and not for limitation.

FIGS. 1A-1C include schematics of exemplary lentivirus systems. FIG. 1A includes a schematic of an exemplary first generation lentivirus system as provided herein. FIG. 1B includes a schematic of an exemplary second generation lentivirus system as provided herein.

FIG. 1C includes a schematic of an exemplary third generation lentivirus system as provided herein. Exemplary fourth generation lentivirus systems for use in technologies described herein are not shown.

FIG. 2 includes a schematic representing an exemplary process for producing lentiviral vectors including a plurality of genomes. As shown, a lentiviral vector produced by the process would include two genomes.

FIG. 3 includes a schematic representing an exemplary process for producing lentiviral vectors including a single genome (“Two LVs”) and a schematic representing an exemplary process for producing lentiviral vectors including a plurality of genomes (“vCAR”). FIG. 3 also includes dot plots showing the expression levels of a CD22 CAR or a CD19 CAR (which were determined using flow cytometry) in primary T cells that were transduced with two single-genome lentiviral vectors or dual-genome lentiviral vectors.

FIG. 4 includes three dot plots representing expression levels of a CD22 CAR or a CD19 CAR (which were determined using flow cytometry) in primary T cells that were transduced with lentiviral vectors having different ratios of genomes including a CD19CAR and genomes including a CD22CAR.

FIG. 5 includes five dot plots representing expression levels of a CD22 CAR or a CD19 CAR (which were determined using flow cytometry) in primary T cells that were transduced with lentiviral vectors having different ratios of genomes including a CD19 CAR and genomes including a CD22 CAR. The y-axis shows the expression level of the CD22 and the x-axis shows the expression level of the CD19 CAR.

FIGS. 6A and 6B demonstrate control of Nalm6 tumor cells by CAR-T cells produced by transduction with lentiviral vectors comprising two genomes. FIG. 6A shows control of tumor cells in dose responsive manner using a luciferase toxicity assay. FIG. 6B shows control of tumor cells in a time dependent manner using an Incucyte assay.

FIGS. 7A, 7B, and 7C demonstrate in vivo control of Nalm6 tumor cells by CAR-T cells produced by transduction with lentiviral vectors comprising two genomes. FIG. 7A shows a time course of the tumor challenge for each treatment condition. FIG. 7B shows images of the challenged mice. FIG. 7C provides the calculated area under the curve through day 30 for each treatment condition.

FIG. 8 demonstrates control of Nalm6 tumor cells in a model of antigen escape using CAR-T cells produced by transduction with lentiviral vectors comprising two genomes.

FIG. 9 includes five plots representing expression levels of a BCMA CAR or a GPRC5D CAR (which were determined using flow cytometry) in primary T cells that were transduced with lentiviral vectors having different ratios of genomes including a BCMA CAR and genomes including a GPRC5D CAR compared to primary T cells simultaneously transduced with two single-genome lentiviral vectors (2 LVs). The y-axis shows the expression level of the GPRC5D and the x-axis shows the expression level of the BCMA CAR.

### CERTAIN DEFINITIONS

As used herein, the singular terms “a,” “an,” and “the” include the plural reference unless the context clearly indicates otherwise.

As used herein, “and/or” refers to and encompasses any and all possible combinations of one or more of the associated listed items, as well as the lack of combinations when interpreted in the alternative (“or”). Moreover, the present invention also contemplates that in some embodiments of the invention, any feature or combination of features set forth herein can be excluded or omitted.

Unless the context requires otherwise, the terms “comprise,” “comprises,” and “comprising,” or similar terms are intended to mean a non-exclusive inclusion, such that a recited list of elements or features does not include those stated or listed elements solely, but may include other elements or features that are not listed or stated.

As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%. 14%. 13%. 12%. 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). In certain embodiments, the term “approximately” or “about” is meant to encompass variations of ±20% or ±10%, more preferably ±5%, even more preferably ±1%, and still more preferably ±0.1% from the specified value, as such variations are appropriate to perform the disclosed methods.

Unless otherwise indicated, nucleic acids are written left to right in the 5′ to 3′ orientation; and amino acid sequences are written left to right in amino to carboxy orientation, respectively.

It is to be understood that this disclosure is not limited to the particular methodology, protocols, and reagents described, as these may vary, depending upon the context in which they are used by those skilled in the art.

As used herein, a “lentiviral vector” or LV refers to a viral vector that contains lentiviral nucleic acid or is derived from a lentivirus. A lentiviral vector includes the following components: a vector genome (lentivirus nucleic acid), a nucleocapsid encapsidating the nucleic acid, and a membrane surrounding the nucleocapsid. Typically, a lentiviral vector contains sufficient lentiviral genetic information to allow packaging of an RNA genome, in the presence of packaging components, into a viral vector capable of infecting a target cell. Infection of the target cell may include reverse transcription and integration into the target cell genome. A lentiviral vector may be a recombinant lentiviral vector that is replication defective and lacks genes essential for replication, such as a functional gag-pol and/or env gene and/or other genes essential for replication. A lentiviral vector also may be a self-inactivating (SIN) vector.

The “Lentiviral genome”, also herein identified as “vector genome” in the context of the disclosure refers to a nucleic acid sequence, which is possibly encapsidated into a lentiviral vector, for example (i) when present in producer cells as a ribonucleic molecule, or when present in the “lentiviral vector” or (ii) when present as a ribonucleic molecule in a cell transduced by a “lentiviral vector”. In the context of the invention, the “lentiviral vector genome” contains all the cis- and trans-active elements necessary for its proper production and encapsidation, including but not limited to the long terminal repeats (LTR) and the psi encapsidation signal. It also contains one or several cassette(s) encoding the gene of interest or transgene, said cassette possibly containing, as understood in the present disclosure, any element involved in the transcriptional or post-transcriptional regulation of the coding sequence. Preferably, the recombinant lentiviral vector genome is devoid of the gag, pol and/or env gene, even more preferably of the gag, pol and env genes. At least one of the gag, pol and env genes, as well as any fragments thereof, can intentionally be reintroduced as a “transgene” in the vector genome, for example in the context of vaccination. Typically, the gag gene may be reintroduced. The “vector cassette” in the context of the disclosure allows the expression of a “vector genome” that will be encapsidated in the lentiviral vector in producer cells. The “vector cassette”, in the context of the disclosure, is to be present transiently or constitutively (i.e. a transformed cell containing an integrated transcomplementation cassette) and expressed in a constitutive or regulated (for example inducible) manner, in the producer cells to produce lentiviral vector genomes typically using a plasmid, an artificial chromosome, a viral vector genome (for example a baculoviral or adenoviral vector genome), a transposon, or any combinations thereof.

As used herein, “transgene” or “payload gene” generally refers to any nucleic acid sequence of interest. The terms “transgene” and “payload gene” are used interchangeably herein unless context indicates otherwise. This is typically a sequence encoding a peptide, for example an enzyme, a transcription factor, a growth factor, a trophic factor, an hormone, a cytokine, an antibody, a receptor, a chimeric antigen receptor (CAR), a differentiation factor, a colony stimulating factor, a suicide protein, a cell-cycle modifying protein, an anti-proliferative protein, a nuclease, a recombinase, a transposase, a neurotransmitter or a precursor thereof. It can be part of an RNA or of a DNA molecule. Preferably, it is a sequence deriving from a cDNA, a gDNA, a RNA, a synthetic nucleic acid, or a combination thereof. The context will indicate whether the term “transgene” or “payload gene” refers to a DNA or a RNA sequence. Typically, the transgene includes a sequence encoding a product of interest. Furthermore, the transgene can include one or more transcription termination regions, typically a polyadenylation signal. The “transgene” or “payload gene” may also typically refer to a non-coding sequence. The transgene can be for example selected from a guide RNA, a catalytic nucleic acid (for example a ribozyme), an interfering nucleic acid, an antisense nucleic acid, an aptamer, a miRNA or a decoy RNA.

The term “cassette” or “expression cassette” in the context of the present invention is to be understood as a nucleic acid sequence possibly involved in the production of one or several RNAs and/or of one or several peptides or polypeptides. The cassette or expression cassette consists in possibly transcribed sequences, for instance but not limited to a gene or a combination of genes, which are possibly associated with functional transcription element(s), namely “promoter(s)”, it possibly contains additional sequences such as leader and trailer sequences, introns and sequences involved in the regulation or modulation of transcription such as inhibitor, enhancer, stabilizer, internal ribosomal entry site (IRES). The cassette or expression cassette can typically be transferred into target cells for example as part of a plasmid, an artificial chromosome, a viral vector genome (for example a baculoviral or adenoviral vector genome), or of a transposon. This cassette is then possibly expressed in the target cells. When the cassette or expression cassette is a DNA molecule or part of a DNA molecule, “expression” is understood as transcription, i.e. production of a polyadenylated messenger RNA and in case of a coding mRNA, its potential subsequent translation and production of a peptide. When the cassette is a RNA molecule or part of a RNA molecule, “expression” refers to the mRNA translation into a peptide. Alternatively, when the cassette contains a non-coding RNA (for example a decoy RNA, miRNA, a ribozyme, etc.), “expression” refers to delivery of the RNA into the target cell and its subsequent processing as a catalytic, decoy or interfering RNA.

The term “construct” refers to any polynucleotide that contains a recombinant nucleic acid molecule. A construct is present in a vector (e.g., a bacterial vector, a viral vector) or is integrated into a genome. A “vector” is a nucleic acid molecule that is capable of introducing a specific nucleic acid sequence into a cell or into another nucleic acid sequence, or as a means of transporting another nucleic acid molecule. Vectors are, for example, plasmids, cosmids, viruses, an RNA vector, or a linear or circular DNA or RNA molecule that may include chromosomal, non-chromosomal, semi-synthetic, or synthetic nucleic acid molecules. Exemplary vectors are those capable of autonomous replication (episomal vector), capable of delivering a polynucleotide to a cell genome (e.g., viral vector), or capable of expressing nucleic acid molecules to which they are linked (expression vectors).

As used herein, a “promoter” refers to a cis-regulatory DNA sequence that, when operably linked to a gene coding sequence, drives transcription of the gene. The promoter may comprise a transcription factor binding sites. In some embodiments, a promoter works in concert with one or more enhancers which are distal to the gene.

As used herein, “operably linked” or “operably associated” includes reference to a functional linkage of at least two sequences. For example, operably linked includes linkage between a promoter and a second sequence, wherein the promoter sequence initiates and mediates transcription of the DNA sequence corresponding to the second sequence. Operably associated includes linkage between an inducing or repressing element and a promoter, wherein the inducing or repressing element acts as a transcriptional activator of the promoter. As used herein, “fusogen” refers to an agent or molecule that creates an interaction between two membranes, including membrane enclosed lumens. In embodiments, the fusogen facilitates fusion of the membranes. In other embodiments, the fusogen creates a connection, e.g., a pore, between two membranes or lumens (e.g., a lumen of a retroviral vector and a cytoplasm of a target cell). In some embodiments, the fusogen comprises a complex of two or more proteins, e.g., wherein neither protein has fusogenic activity alone. In some embodiments, the fusogen comprises a targeting domain.

As used herein, “G protein” refers to a henipavirus envelope attachment glycoprotein G or biologically active portion thereof. “F protein” refers to a henipavirus fusion protein F or biologically active portion thereof. In some embodiments, the F and G proteins are from a Hendra (HeV) or a Nipah (NiV) virus, and are a wild-type protein or are a variant thereof that exhibits reduced binding for the native binding partner. The F (fusion) and G (attachment) glycoproteins mediate cellular entry of Nipah virus. The G protein initiates infection by binding to the cellular surface receptor ephrin-B2 (EphB2) or EphB3. The subsequent release of the viral genome into the cytoplasm is mediated by the action of the F protein, which induces the fusion of the viral envelope with cellular membranes. In some embodiments, the efficiency of transduction of targeted viral vector is improved by engineering hyperfusogenic mutations in one or both of the F protein (such as NiV-F) and G protein (such as NiV-G).

As used herein, a “re-targeted fusogen” refers to a fusogen that comprises a targeting moiety having a sequence that is not part of the naturally-occurring form of the fusogen. In embodiments, the fusogen comprises a different targeting agent relative to the targeting agent in the naturally-occurring form of the fusogen. In embodiments, the naturally-occurring form of the fusogen lacks a targeting domain, and the re-targeted fusogen comprises a targeting agent that is absent from the naturally-occurring form of the fusogen. In embodiments, the fusogen is modified to comprise a targeting agent. In embodiments, the fusogen comprises one or more sequence alterations outside of the targeting agent relative to the naturally-occurring form of the fusogen, e.g., in a transmembrane domain, fusogenically active domain, or cytoplasmic domain.

As used herein, a “targeted envelope protein” refers to a polypeptide that contains a henipavirus G protein (G protein) attached to a single domain antibody (sdAb) variable domain, such as a VL or VH sdAb, a scFv, a nanobody, a camelid VHH domain, a shark IgNAR, or fragments thereof, that target a molecule on a desired cell type. In some such embodiments, the attachment is directly or indirectly via a linker, such as a peptide linker. The “targeted envelope protein” may also be referred to as a “fusion protein” comprising the G protein and antibodies or antigen binding fragments of the disclosure in which the antibody or antigen binding fragment is fused to the C-terminus of the G protein or a biologically active portion thereof.

As used herein, a “target cell” refers to a cell of a type to which it is desired that a targeted viral vector delivers an exogenous agent. In embodiments, a target cell is a cell of a specific tissue type or class. In some embodiments, the targeting agent or fusogen, e.g., re-targeted fusogen, leads to preferential delivery of the exogenous agent to a target cell compared to a non-target cell.

As used herein a “non-target cell” refers to a cell of a type to which it is not desired that a targeted viral vector delivers an exogenous agent. In some embodiments, a non-target cell is a cell of a specific tissue type or class. In some embodiments, the targeting agent or fusogen, e.g., re-targeted fusogen leads to lower delivery of the exogenous agent to a non-target cell compared to a target cell.

As used herein, “affinity” refers to the strength of the sum total of noncovalent interactions between a single binding site of a molecule (e.g., a receptor) and its binding partner (e.g., a ligand). The affinity of a molecule for its partner can generally be represented by the equilibrium dissociation constant (K_{D}) (or its inverse equilibrium association constant, K_{A}). Affinity can be measured by common methods known in the art, including those described herein. See, for example, Pope M. E., Soste M. V., Eyford B. A., Anderson N. L., Pearson T. W., (2009) J. Immunol. Methods. 341(1-2):86-96 and methods described therein.

As used herein, the terms “percent identity” and “% identity,” as applied to nucleic acid or polynucleotide sequences, refer to the percentage of residue matches between at least two nucleic acid or polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences.

Percent identity between nucleic acid or polynucleotide sequences may be determined using a suite of commonly used and freely available sequence comparison algorithms provided by the National Center for Biotechnology Information (NCBI) Basic Local Alignment Search Tool (BLAST) (Altschul, S. F. et al. (1990) J. Mol. Biol. 215:403-410), which is available from several sources, including the NCBI, Bethesda, Md., and on the Internet at http://www.ncbi.nlm.nih.gov/BLAST/.

Nucleic acid or polynucleotide sequences that do not show a high degree of identity may nevertheless encode similar amino acid sequences due to the degeneracy of the genetic code. It is understood that changes in a nucleic acid sequence can be made using this degeneracy to produce multiple nucleic acid sequences that all encode substantially the same protein. Specifically, degenerate codon substitutions may be achieved by generating sequences in which the third position of one or more selected (or all) codons is substituted with mixed-base and/or deoxyinosine residues (Batzer et al. (1991) Nucleic Acid Res 19:5081; Ohtsuka et al. (1985) J Biol Chem 260:2605-2608; Cassol et al. (1992); Rossolini et al. (1994) Mol Cell Probes 8:91-98).

As used herein, “percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN (DNASTAR) software. Those skilled in the art can determine appropriate parameters for measuring alignment, including any algorithms needed to achieve maximal alignment over the full length of the sequences being compared.

An amino acid substitution refers to the replacement of one amino acid in a polypeptide with another amino acid. Exemplary substitutions are shown in Table 1. Amino acid substitutions may be introduced into a protein of interest and the products screened for a desired activity, for example, retained/improved biological activity.

TABLE 1
Exemplary Amino Acid Substitutions

| Original Residue | Exemplary Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class. The term, “corresponding to” with reference to nucleotide or amino acid positions of a sequence, such as set forth in the Sequence Listing, refers to nucleotides or amino acid positions identified upon alignment with a target sequence based on structural sequence alignment or using a standard alignment algorithm, such as the GAP algorithm. For example, corresponding residues of a similar sequence (e.g., a fragment or species variant) can be determined by alignment to a reference sequence by structural alignment methods. By aligning the sequences, one skilled in the art can identify corresponding residues, for example, using conserved and identical amino acid residues as guides.

By “wild type” or “WT” or “native” herein is meant an amino acid sequence that is found in nature, including allelic variations. A wild type protein or polypeptide has an amino acid sequence that has not been intentionally modified.

As used herein a “biologically active portion,” such as with reference to a protein such as a G protein or an F protein, refers to a portion of the protein that exhibits or retains an activity or property of the full-length of the protein. For example, a biologically active portion of an F protein retains fusogenic activity in conjunction with the G protein when each are embedded in a lipid bilayer. A biologically active portion of the G protein retains fusogenic activity in conjunction with an F protein when each is embedded in a lipid bilayer. In some embodiments, the retained activity includes 10%-150% or more of the activity of a full-length or wild-type F protein or G protein. Examples of biologically active portions of F and G proteins include truncations of the cytoplasmic domain, e.g., truncations of up to 1, 2, 3, 4, 5, 6, 7, 8 9, 10, 11, 12, 13, 14, 15, 20, 22, 25, 30, 33, 34, 35, or more contiguous amino acids, see e.g. Khetawat and Broder 2010 Virology Journal 7:312; Witting et al. 2013 Gene Therapy 20:997-1005; published international; patent application No. WO/2013/148327.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, for example, in the case of an RNA polynucleotide. Thus, a DNA polynucleotide that is contained in a vector inside a host cell may be referred to as “isolated”.

The term “effective amount” as used herein means an amount of a pharmaceutical composition which is sufficient enough to significantly and positively modify the symptoms and/or conditions to be treated (e.g., provide a positive clinical response). The effective amount of an active ingredient for use in a pharmaceutical composition will vary with the particular condition being treated, the severity of the condition, the duration of treatment, the nature of concurrent therapy, the particular active ingredient(s) being employed, the particular pharmaceutically-acceptable excipient(s) and/or carrier(s) utilized, and like factors with the knowledge and expertise of the attending physician.

As used herein, a composition refers to any mixture of two or more products, substances, or compounds, including cells. It may be a solution, a suspension, liquid, powder, a paste, aqueous, non-aqueous or any combination thereof.

The term “hypoimmunogenicity,” “hypoimmunogeneic,” “hypoimmunogenic,” “hypoimmunity,” or “hypoimmune” is used interchangeably to describe a cell being less prone to immune rejection by a subject into which such cell is transplanted. For example, relative to an unaltered or unmodified wild-type cell, such a hypoimmunogenic cell is about 2.5%, 5%, 10%, 20%, 30%, 40%, 50%, 60%, 70%, 80%, 90%, 95%, 97.5%, 99% or more less prone to immune rejection by a subject into which such cell is transplanted. In some examples described herein, genome editing technologies are used to modulate the expression of MHC I and/or MHC II genes, and thus, to generate a hypoimmunogenic cell. In other examples described herein, a tolerogenic factor is introduced into a cell and when expressed can modulate or affect the ability of the cell to be recognized by host immune system and thus confer hypoimmunogenicity. Hypoimmunogenicity of a cell is determined by evaluating the cell's ability to elicit adaptive and innate immune responses. Such immune response can be measured using assays recognized by those skilled in the art, for example, by measuring the effect of a hypoimmunogenic cell on T cell proliferation, T cell activation, T cell killing, NK cell proliferation, NK cell activation, and macrophage activity. Hypoimmunogenic cells may undergo decreased killing by T cells and/or NK cells upon administration to a subject or show decreased macrophage engulfment compared to an unmodified or wildtype cell. In some embodiments, a hypoimmunogenic cell elicits a reduced or diminished immune response in a recipient subject compared to a corresponding unmodified wild-type cell. In some embodiments, a hypoimmunogenic cell is nonimmunogenic or fails to elicit an immune response in a recipient subject.

As used herein, the term “pharmaceutically acceptable” refers to a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

As used herein, the term “pharmaceutical composition” refers to a mixture of at least one viral vector with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to, intravenous, oral, aerosol, parenteral, ophthalmic, pulmonary and topical administration.

A “disease” or “disorder” as used herein refers to a condition where treatment is needed and/or desired.

As used herein, the terms “treat,” “treating,” or “treatment” refer to ameliorating a disease or disorder, e.g., slowing or arresting or reducing the development of the disease or disorder or reducing at least one of the clinical symptoms thereof. For purposes of this disclosure, ameliorating a disease or disorder can include obtaining a beneficial or desired clinical result that includes, but is not limited to, any one or more of: alleviation of one or more symptoms, diminishment of extent of disease, preventing or delaying spread (for example, metastasis, for example metastasis to the lung or to the lymph node) of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total).

As used herein, the terms “nuclear export sequence” (NES) or “nuclear export signal” (NES) refer to a nuclear export signal or other sequence or domain that is present in a protein and capable of targeting the protein for export from the cell nucleus to the cytoplasm through the nuclear pore complex using nuclear transport. A nuclear export domain can be fused (e.g., fused in-frame) with a polypeptide.

As used herein, the terms “nuclear localization sequence” (NLS) or “nuclear localization sequence” (NLS) refer to a nuclear localization signal or other sequence or domain that is present in a protein and capable of targeting the protein for import from the cytoplasm to the cell nucleus through the nuclear pore complex using nuclear transport. A nuclear localization can be fused (e.g., fused in-frame) with a polypeptide.

The term “tolerogenic factor” as used herein includes hypoimmunity factors, complement inhibitors, and other factors that modulate or affect (e.g., reduce) the ability of a cell to be recognized by the immune system of a host or recipient subject upon administration, transplantation, or engraftment. Tolerogenic factors include but are not limited to CD16, CD24, CD35, CD39, CD46, CD47, CD52, CD55, CD59, CD200, CCL22, CTLA4-Ig, C1 inhibitor, FASL, IDO1, HLA-C, HLA-E, HLA-E heavy chain, HLA-G, IL-10, IL-35, PD-L1, Serpinb9, CCl21, Mfge8, A20/TNFAIP3, CCL21, CD16 Fe receptor, CD27, CR1, DUX4, H2-M3 (HLA-G), HLA-F, IL15-RF, MANF, IL-39, and B2M-HLA-E.

As used herein, the term “antibody” refers to an immunoglobulin molecule, or fragment thereof, that binds specifically to an epitope (e.g., of an antigen). Naturally-occurring human antibodies typically include two identical heavy chains and two identical light chains, each of which includes a variable domain and a constant domain. The constant domain defines the antibody isotype (IgG, IgA, TgM, IgE, etc). Those skilled in the art aware that different animal species utilize different antibody structures in nature. For example, camelid antibodies are single chain antibodies. Those skilled in the art will further be aware that antibody variable domains are typically characterized by framework region (FR) sequences and complement-determining-region (CDR) sequences. Each variable domain typically includes three CDRs—CDR1, CDR2, and CDR3, which together contribute to specificity and/or affinity of epitope binding. In some embodiments, antibodies may be produced in or by organisms. In some embodiments, antibodies may be produced in or by cells in vitro (e.g., by hybridomas and/or by engineered cells). In some embodiments, antibody fragment(s) (e.g., as may be produced by cleavage or recombinantly) may be generated and/or utilized in accordance with the present disclosure. Those skilled in the art are aware that a variety of technologies have been developed to incorporate binding features (e.g., one or more CDRs and/or FR sequences, and in particular sets of 3 CDRs, optionally together with FR sequences) into new contexts. In some embodiments, CDR sequences may be maintained and other elements changed—e.g., as is done in humanization. Alternatively or additionally, in some embodiments, variable regions may be associated with alternative constant regions (e.g., with constant regions from a different organism and/or that include one or more particular sequence features or elements desired, for example, to impart a particular attribute to the antibody agent. Those skilled in the art are further aware of a variety of technologies commonly utilized to associate binding features of two or more different antibodies with one another—e.g., in a single multispecific (most commonly bi-specific) agent. Various known formats of antibody agents (i.e., agents that incorporate antibody binding sequences such as one or more CDRs, or a full set of CDRs) include, for instance: antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies; Adnectins®; Affilins®; Trans-Bodies®; Affibodies®; TrimerX®; MicroProteins; Fynomers®, Centyrins®; KALBITOR®s, Zybodies®, etc.

The term “antigen”, as used herein, refers to an agent that elicits an immune response; and/or (ii) an agent that binds to a T cell receptor (e.g., when presented by an MHC molecule) or to an antibody. In some embodiments, an antigen elicits a humoral response (e.g., including production of antigen-specific antibodies); in some embodiments, an antigen elicits a cellular response (e.g., involving T-cells whose receptors specifically interact with the antigen). In some embodiments, and antigen binds to an antibody and may or may not induce a particular physiological response in an organism. In general, an antigen may be or include any chemical entity such as, for example, a small molecule, a nucleic acid, a polypeptide, a carbohydrate, a lipid, a polymer (in some embodiments other than a biologic polymer [e.g., other than a nucleic acid or amino acid polymer) etc. In some embodiments, an antigen is or comprises a polypeptide. In some embodiments, an antigen is or comprises a glycan. Those of ordinary skill in the art will appreciate that, in general, an antigen may be provided in isolated or pure form, or alternatively may be provided in crude form (e.g., together with other materials, for example in an extract such as a cellular extract or other relatively crude preparation of an antigen-containing source). In some embodiments, antigens utilized in accordance with the present invention are provided in a crude form. In some embodiments, an antigen is a recombinant antigen.

As used herein, “antigen binding fragment” or “antibody fragment” refers to a portion of an immunoglobulin molecule that retains the heavy chain and/or the light chain antigen binding site, such as a heavy chain complementarity determining regions (HCDR) 1 (HCDR1), 2 (HCDR2), and 3 (HCDR3), a light chain complementarity determining regions (LCDR) 1 (LCDR1), 2 (LCDR2), and 3 (LCDR3), a heavy chain variable region (VH), or a light chain variable region (VL). Antibody fragments include a Fab fragment (a monovalent fragment consisting of the VL or the VH); a F(ab) 2 fragment (a bivalent fragment comprising two Fab fragments linked by a disulfide bridge at the hinge region); a Fd fragment consisting of the VH and CH1 domains; a Fv fragment consisting of the VL and VH domains of a single arm of an antibody; a dAb fragment, which consists of a VH domain; and a variable domain (VHH) from, e.g., human or camelid origin. VH and VL domains are engineered and linked together via a synthetic linker to form various types of single chain antibody designs in which the VH/VL domains pair intramolecularly, or intermolecularly in those embodiments in which the VH and VL domains are expressed by separate single chain antibody constructs, to form a monovalent antigen binding site, such as a single-chain Fv (scFv) or diabody. These antibody fragments are obtained using well known techniques and the fragments are characterized in the same manner as are intact antibodies.

An antibody variable region consists of a “framework” region interrupted by three “antigen binding sites.” The antigen binding sites are defined using various terms, including, for example (i) “Complementarity Determining Regions” (CDRs), three in the VH (HCDR1, HCDR2, HCDR3) and three in the VL (LCDR1, LCDR2, LCDR3) (Wu and Kabat, J Exp Med 132:211-50, 1970; Kabat et al, Sequences of Proteins of Immunological Interest, 5th Ed. Public Health Service, National Institutes of Health, Bethesda, Md., 1991), and (ii) “Hypervariable regions,” “HVR,” or “HV,” three in the VH (H1, H2, H3) and three in the VL (L1, L2, L3) (Chothia and Lesk Mol Biol 196:901-17, 1987). Other terms include “IMGT-CDRs” (Lefranc et al., Dev Comparat Inmunol 27:55-77, 2003) and “Specificity Determining Residue Usage” (SDRU) (Almagro Mol Recognit, 17:132-43, 2004). The International ImMunoGeneTics (IMGT) database (http://www_imgt org) provides a standardized numbering and definition of antigen-binding sites. The correspondence between CDRs, HVs, and IMGT delineations is described in Lefranc et al., Dev Comparat Immunol 27:55-77, 2003.

The term “framework,” or “FR” or “framework sequence” refers to the remaining sequences of a variable region other than those sequences defined to be antigen binding sites. Because the antigen binding site can be defined by various terms as described above, the exact amino acid sequence of a framework depends on how the antigen-binding site was defined.

A “binding domain,” also referred to as a “binding region,” refers to an antibody or portion thereof that possesses the ability to specifically and non-covalently associate, unite, or combine with a target. A binding domain includes any naturally occurring, synthetic, semi-synthetic, or recombinantly produced binding partner for a biological molecule, a molecular complex, or other target of interest. Exemplary binding domains include receptor ectodomains, ligands, scFvs, disulfide linked Fvs, sdAbs, VHH antibodies, Fab fragments, Fab′ fragments, F(ab′)2 fragments, diabodies, or other synthetic polypeptides selected for their specific ability to bind to a biological molecule, a molecular complex, or other target of interest.

The term “CDR” denotes a complementarity determining region as defined by at least one manner of identification to one of skill in the art. The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme); Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 Jan. 27(1):55-77 (“IMGT” numbering scheme); Honegger A and Pluckthun A, “Yet another numbering scheme for immunoglobulin variable domains: an automatic modeling and analysis tool,” J Mol Biol, 2001 Jun. 8; 309(3):657-70, (“Aho” numbering scheme); and Martin et al., “Modeling antibody hypervariable loops: a combined algorithm,” PNAS, 1989, 86(23):9268-9272, (“AbM” numbering scheme).

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme. The AbM scheme is a compromise between Kabat and Chothia definitions based on that used by Oxford Molecular's AbM antibody modeling software.

In some embodiments, CDRs can be defined in accordance with any of the Chothia numbering schemes, the Kabat numbering scheme, the IMGT numbering scheme, a combination of Kabat, IMGT, and Chothia, the AbM definition, and/or the contact definition. A sdAb variable domain comprises three CDRs, designated CDR1, CDR2, and CDR3. For CDR-H1, residue numbering is listed using both the Kabat and Chothia numbering schemes. FRs are located between CDRs, for example, with FR-H1 located before CDR-H1, FR-H2 located between CDR-H1 and CDR-H2, FR-H3 located between CDR-H2 and CDR-H3 and so forth. It is noted that because the shown Kabat numbering scheme places insertions at H35A and H35B, the end of the Chothia CDR-H1 loop when numbered using the shown Kabat numbering convention varies between H32 and H34, depending on the length of the loop.

Thus, unless otherwise specified, a “CDR” or “complementary determining region,” or individual specified CDRs (e.g., CDR-H1, CDR-H2, CDR-H3), of a given antibody or region thereof, such as a variable region thereof, should be understood to encompass a (or the specific) complementary determining region as defined by any of the aforementioned schemes. For example, where it is stated that a particular CDR (e.g., a CDR-H3) contains the amino acid sequence of a corresponding CDR in a given sdAb amino acid sequence, it is understood that such a CDR has a sequence of the corresponding CDR (e.g., CDR-H3) within the sdAb, as defined by any of the aforementioned schemes. It is understood that any antibody, such as a sdAb, includes CDRs and such are identified according to any of the other aforementioned numbering schemes or other numbering schemes known to a skilled artisan.

As used herein, “Fv” refers to the minimum antibody fragment which contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the VH-VL dimer. Collectively, the six hypervariable regions confer antigen-binding specificity to the antibody. However, even a single variable domain (or half of an Fv comprising only three hypervariable regions specific for an antigen) may have the ability to recognize and bind an antigen, although at a lower affinity than the entire binding site.

As used herein, “single-chain Fv” or “scFv” antibody fragments comprise the VH and VL domains of an antibody, wherein these domains are present in a single polypeptide chain. Preferably, the Fv polypeptide further comprises a polypeptide linker between the VH and VL domains which enables the scFv to form the desired structure for antigen binding. For a review of scFv see Pluckthun in The Pharmacology of Monoclonal Antibodies, vol. 113, Rosenburg and Moore eds., Springer-Verlag, New York, pp. 269-315 (1994).

As used herein, “VHH” or “VHH antibodies” refer to single domain antibodies that consist of the variable region of a heavy chain of an IgG antibody. For example, the terms “VHH” and “VHH antibody” can refer to the antigen binding domain of a heavy chain IgG (hcIgG) molecule produced by a Camelidae family mammal (e.g., llamas, camels, and alpacas).

As used herein, the term “specifically binds” to a target molecule, such as an antigen, means that a binding molecule, such as a single domain antibody (sdAb), reacts or associates more frequently, more rapidly, with greater duration, and/or with greater affinity with a particular target molecule than it does with alternative molecules. A binding molecule, such as a sdAb or scFv, “specifically binds” to a target molecule if it binds with greater affinity, avidity, more readily, and/or with greater duration than it binds to other molecules. It is understood that a binding molecule, such as a sdAb or scFv, that specifically binds to a first target may or may not specifically bind to a second target. As such, “specific binding” does not necessarily require (although it can include) exclusive binding.

As used herein, the term “cell surface molecule” means a molecule that is present on the outer surface of a cell. In some embodiments, the cell surface molecule is an antigen, as herein defined and disclosed. In some embodiments, the cell surface molecule is, for example, a peptide, glycopeptide, polypeptide, glycopolypeptide, polynucleotide, polysaccharide, lipid, or the like that is not immunogenic.

As used herein, the term “crude drug substance” is an intermediate composition generated in the production of a drug substance. In some embodiments, an active ingredient (e.g., viral vectors) in a crude drug substance have been enriched from other components (e.g., producer cell components, e.g., producer cell DNA and/or protein) that the active ingredient has been associated with during a production process. A crude drug substance often needs further processing to purify, isolate, or otherwise enrich the active ingredient prior to being classified as a drug substance or being incorporated into a drug product.

As used herein, the term “drug substance” is an active ingredient (e.g., viral vectors) that is intended to furnish pharmacological activity or other direct effect in the diagnosis, cure, mitigation, treatment, or prevention of disease or to affect the structure or any function of a subject's body, but does not include intermediates used in the synthesis of such ingredient. A drug substance may need further processing to become a “drug product,” which is a finished dosage form (e.g., tablet or solution) to be administered to a subject. However, a drug substance does not require further processing to purify, isolate, or otherwise enrich the active ingredient prior to incorporation into a drug product.

In general, the term “engineered” refers to the aspect of having been manipulated by the hand of man. For example, a polynucleotide is considered to be “engineered” when two or more sequences that are not linked together in that order in nature are manipulated by the hand of man to be directly linked to one another in the engineered polynucleotide and/or when a particular residue in a polynucleotide is non-naturally occurring and/or is caused through action of the hand of man to be linked with an entity or moiety with which it is not linked in nature. For example, an engineered polynucleotide comprises a regulatory sequence that is found in nature in operative association with a first coding sequence but not in operative association with a second coding sequence, is linked by the hand of man so that it is operatively associated with the second coding sequence. Comparably, a polypeptide may be considered to be “engineered” if encoded by or expressed from an engineered polynucleotide, and/or if produced other than natural expression in a cell. Analogously, a cell or organism is considered to be “engineered” if it has been subjected to a manipulation, so that its genetic, epigenetic, and/or phenotypic identity is altered relative to an appropriate reference cell such as otherwise identical cell that has not been so manipulated. In some embodiments, the manipulation is or comprises a genetic manipulation, so that its genetic information is altered (e.g., new genetic material not previously present has been introduced, for example by transformation, mating, somatic hybridization, transfection, transduction, or other mechanism, or previously present genetic material is altered or removed, for example by substitution or deletion mutation, or by mating protocols). In some embodiments, an engineered cell is one that has been manipulated so that it contains and/or expresses a particular agent of interest (e.g., a protein, a nucleic acid, and/or a particular form thereof) in an altered amount and/or according to altered timing relative to such an appropriate reference cell. As is common practice and is understood by those in the art, progeny of an engineered polynucleotide or cell are typically still referred to as “engineered” even though the actual manipulation was performed on a prior entity.

As used herein, “excipient” refers to a non-therapeutic agent that may be included in a pharmaceutical composition, for example to provide or contribute to a desired consistency or stabilizing effect.

As used herein, “improved,” “increased,” “decreased” or “reduced”, or grammatically comparable comparative terms, indicate values that are relative to a comparable reference measurement. For example, in some embodiments, an assessed value achieved with a method of interest may be “improved” relative to that obtained with a comparable reference method. Alternatively or additionally, in some embodiments, an assessed value achieved in a method of interest may be “improved” relative to that obtained in the same method under different conditions (e.g., prior to or after an event or step). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance.

As used herein, “nucleic acid” in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid residue (e.g., a nucleotide and/or nucleoside); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110,120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is partly or wholly single stranded; in some embodiments, a nucleic acid is partly or wholly double stranded. In some embodiments a nucleic acid has a nucleotide sequence comprising at least one element that encodes, or is the complement of a sequence that encodes, a polypeptide. In some embodiments, a nucleic acid has a functional activity.

As used herein, “plasmid” refers to a nucleic acid molecule capable of transporting another nucleic acid to which it has been linked. Standard techniques may be used for recombinant DNA, oligonucleotide synthesis, and tissue culture and transformation (e.g., transfection, e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose.

As used herein, “polypeptide” refers to a polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a relevant polypeptide may comprise or consist of a fragment of a parent polypeptide. In some embodiments, a useful polypeptide as may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

As used herein, a “producer cell” is a cell capable of producing a viral vector when cultured under appropriate conditions. A number of cells are known to be capable of producing viral vectors, including for example, HEK293 cells, PER.C6 cells, VERO cells, 293T cells, A549 cells, MRC5 cells, HeLa cells, Sf9 cells, and BHK-21 cells.

As used herein, the term “subject” refers an organism, typically a mammal (e.g., a human). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

As used herein, the term “tropism” refers to the ability of a molecule (e.g., a fusogen) or viral vector to interact with molecules (e.g., receptors or antigens) associated with a cell (e.g., in its cell membrane). For example, if a fusogen has a tropism for T cells, the fusogen is able to interact with molecules on the surface of or in the cell membrane of a T cell. In some embodiments, this interaction allows for the fusion of the viral vector with the membrane of the cell and ultimately entry into the cell.

All literature and similar material cited in this application, including, but not limited to, patents, patent applications, articles, books, treatises, and web pages, regardless of the format of such literature and similar materials, are expressly incorporated by reference in their entirety. In the event that one or more of the incorporated literature and similar materials differs from or contradicts this application, including but not limited to defined terms, term usage, described techniques, or the like, this application controls. The section headings used herein are for organizational purposes only and are not to be construed as limiting the subject matter described in any way.

Before the technology is further described, it is to be understood that this technology is not limited to particular embodiments described, as such may, of course, vary. It is also to be understood that the terminology used herein is for the purpose of describing particular embodiments only, and is not intended to be limiting, since the scope of the present disclosure will be limited only by the appended claims. It should also be understood that the headers used herein are not limiting and are merely intended to orient the reader, but the subject matter generally applies to the technology disclosed herein.

### DETAILED DESCRIPTION OF CERTAIN EMBODIMENTS

The present disclosure provides, among other things, an engineered lentiviral vector comprising a plurality (e.g., two or more) of genomes. An engineered lentiviral vector comprising a plurality of genomes can provide benefits, including a simpler and more efficient manner of delivering a plurality of transgenes to a transduced cell and with fewer compositions that may be subject to regulatory review. The present disclosure also provides lentiviral vector systems, e.g., for the production of lentiviral vectors, as well as methods of using the same.

In some embodiments, an engineered lentiviral vector as described herein comprises a first genome and a second genome. In some embodiments, a first genome comprises a first transgene. In some embodiments, a second genome comprises a second transgene. In some embodiments, a first and a second transgene are different.

In order to produce engineered lentiviral vectors as described herein, producer cells are transfected with a lentiviral vector system and cultured under conditions sufficient to produce the lentiviral vectors, as is understood in the art. Lentiviral vector systems comprise one or more nucleic acids for the production of a lentiviral vector. Exemplary nucleic acids in such systems are plasmids. Because lentiviral vectors are often used for delivery of nucleic acids to cells (including in vitro, ex vivo, and in vivo cells), it can be important for the lentiviral vectors to be incapable of replicating. To that end, various generations of lentiviral vector systems have been developed to minimize the production of replication competent lentiviral vectors and increase the safety of lentiviral vector use. A brief description of lentiviral vector systems and certain variations are described below.

### I. Lentiviral Vector Systems

The present disclosure provides, in part, engineered lentiviral vectors comprising a plurality (e.g., two or more) of genomes, and methods of making and using the same. The present disclosure further provides one or more nucleic acids for the production of engineered lentiviral vectors comprising a plurality (e.g., two or more) of genomes. In some embodiments, one or more nucleic acids comprise plasmids for the production of a viral vector. One or more nucleic acids (e.g., plasmids) for the production of a viral vector can include various elements, which can be divided among the one or more nucleic acids (e.g., plasmids) in various ways, as known in the art.

For the production of lentiviral vectors, several lentiviral vector systems have been developed, including first-generation, second-generation, third-generation and more recently fourth-generation vector systems. However, regardless of the generation of the lentiviral vector system, three plasmids are typically utilized: a packaging plasmid, an envelope plasmid, and a transfer plasmid. A transfer plasmid includes genetic material that becomes the genome of produce lentiviral vectors.

A brief description of those systems, including the nucleic acids (e.g., plasmids) is included below.

### A. First Generation

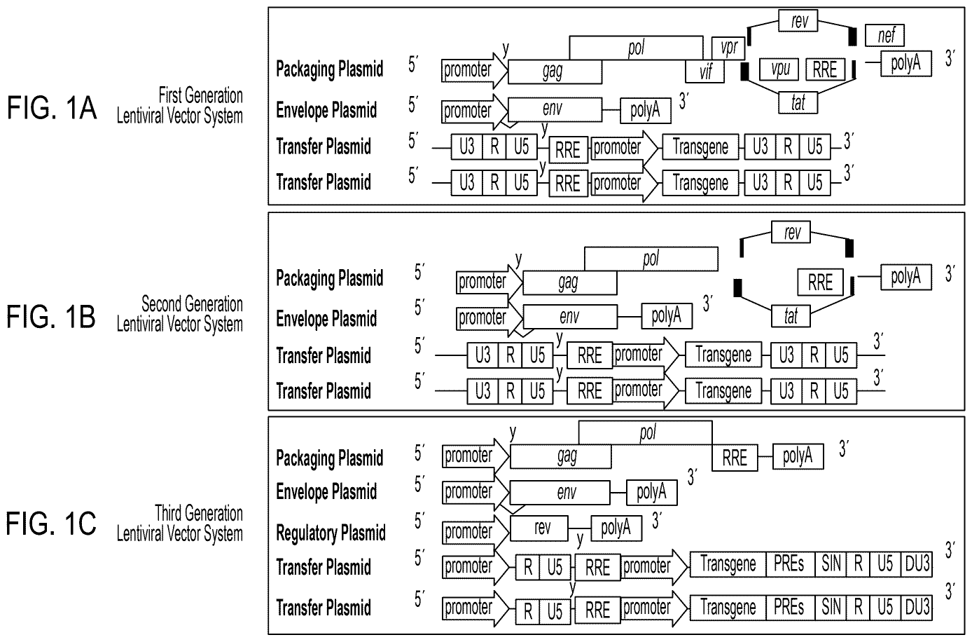

As shown in FIG. 1A, typically a first-generation lentiviral vector system includes three plasmids: a packaging plasmid, an envelope plasmid, and a transfer plasmid. A packaging plasmid of a first-generation lentivirus system usually encodes one or more accessory polypeptides (such as viral infectivity factor (Vif), viral protein r (Vpr), viral protein u (Vpu), and negative factor (Nef)) and one or more polypeptides involved in viral production (such as group-specific antigen (Gag), polymerase (Pol), regulator of virion (Rev), and trans-activator of transcription (Tat) genes). An envelope plasmid of a first-generation lentiviral vector system typically encodes an envelope protein (Env) which is usually the HIV-1 Env glycoprotein or the VSV-G glycoprotein. A first-generation lentiviral vector system can also include a transfer plasmid which typically has a 5′ long terminal repeat (LTR), a Rev responsive element, a sequence for a promoter of interest, a transgene of interest, and a 3′ LTR. The LTRs typically comprise a U3, an R, and a U5 region.

As mentioned, a transfer plasmid can comprise a transgene. A transgene as described herein can encode any gene product (e.g., RNA or polypeptide). Exemplary transgenes and encoded gene products are described herein. The present disclosure provides that lentiviral vector systems that include two or more transfer plasmids so that lentiviral vectors produced from such systems include two or more genomes.

In some embodiments, when a lentiviral vector system disclosed herein is a first-generation lentiviral vector system, one or more envelope plasmids do not encode an Env polypeptide. In some embodiments, one or more envelope plasmids of a lentiviral vector system encode one or more fusogens or a biologically active portions thereof. In some embodiments, a fusogen comprises a glycoprotein, e.g., protein G or portion thereof, and/or protein F or portions thereof. In some embodiments, one or more envelope plasmids encode paramyxovirus glycoprotein G (protein G) or a portion thereof and paramyxovirus fusion protein (protein F) or a portion thereof. In some embodiments, a fusogen comprises a chimeric protein. Various fusogens are contemplated as discussed further herein.

In some embodiments, a lentiviral vector system disclosed herein is a first-generation lentiviral vector system. In some embodiments, a lentivirus system comprises one or more plasmids of a first-generation lentiviral vector system as disclosed herein. In some embodiments, a lentivirus system comprises one or more packaging plasmids, one or more envelope plasmids, and two or more transfer plasmids of a first-generation lentiviral vector system. In some embodiments, a lentivirus system comprises a packaging plasmid, an envelope plasmid and two or more transfer plasmids of a first-generation lentiviral vector system. In some embodiments, a lentivirus system comprises a packaging plasmid, two or more envelope plasmids and two or more transfer plasmids of a first-generation lentiviral vector system.

### B. Second Generation

As shown in FIG. 1B, a second-generation lentiviral vector system also includes three plasmids, similar to the first-generation lentiviral vector system shown in FIG. 1A. The three plasmids of a second-generation lentiviral vector system are: a packaging plasmid, an envelope plasmid and a transfer plasmid. One of the differences in a second-generation lentiviral vector system as compared to the earlier generation is that the packaging plasmid does not encode viral accessory polypeptides Vif, Vpr, Vpu, and Nef. In a second-generation lentiviral vector system, the packaging plasmid only encodes the Gag, Pol, Tat and Rev polypeptides.

As mentioned, a transfer plasmid can comprise a transgene. A transgene as described herein can encode any gene product (e.g., RNA or polypeptide). Exemplary transgenes and encoded gene products are described herein. The present disclosure provides that lentiviral vector systems that include two or more transfer plasmids so that lentiviral vectors produced from such systems include two or more genomes.

In some embodiments, when a lentiviral vector system disclosed herein is a second-generation lentiviral vector system, one or more envelope plasmids do not encode an Env polypeptide. In some embodiments, one or more envelope plasmids of a lentiviral vector system encode one or more fusogens or a biologically active portions thereof. In some embodiments, a fusogen comprises a glycoprotein, e.g., protein G or portion thereof, and/or protein F or portions thereof. In some embodiments, one or more envelope plasmids encode paramyxovirus glycoprotein G (protein G) or a portion thereof and paramyxovirus fusion protein (protein F) or a portion thereof. In some embodiments, a fusogen comprises a chimeric protein. Various fusogens are contemplated as discussed further herein.

In some embodiments, a lentiviral vector system disclosed herein is a second-generation lentiviral vector system as disclosed herein. In some embodiments, a lentivirus system comprises one or more plasmids of a second-generation lentiviral vector system as disclosed herein. In some embodiments, a lentivirus system comprises one or more packaging plasmids, one or more envelope plasmids, and two or more transfer plasmids of a second-generation lentiviral vector system. In some embodiments, a lentivirus system comprises a packaging plasmid, an envelope plasmid and two or more transfer plasmids of a second-generation lentiviral vector system. In some embodiments, a lentivirus system comprises a packaging plasmid, two envelope plasmids and two or more transfer plasmids of a second-generation lentiviral vector system.

### C. Third Generation

Third-generation lentiviral vector systems are newer and were developed to increase the safety of earlier generation vector systems (See FIG. 1C). In the third-generation lentiviral vector system there are four plasmids: a packaging plasmid, an envelope plasmid, a regulatory plasmid, and a transfer plasmid. The envelope plasmid of third-generation lentiviral vector systems is relatively unchanged from envelope plasmids in prior generations in that it encodes an Env polypeptide. The packaging and transfer plasmids have several differences as described herein.

In a third-generation lentiviral vector system, the packaging plasmid only encodes the Gag and Pol polypeptides. A separate plasmid, the regulatory plasmid encodes the Rev polypeptide. The transfer plasmid also includes changes particularly to the LTRs. To enhance safety, the LTRs were modified in the U3 region. A transfer plasmid of a third-generation lentiviral vector system includes LTR regions comprising an R element, a U5 element, an RRE element, a posttranscriptional regulatory elements (PREs), and a self-inactivating (SIN) region.

As mentioned, a transfer plasmid can comprise a transgene. A transgene as described herein can encode any gene product (e.g., RNA or polypeptide). Exemplary transgenes and encoded gene products are described herein. The present disclosure provides that lentiviral vector systems that include two or more transfer plasmids so that lentiviral vectors produced from such systems include two or more genomes.

In some embodiments, when a lentiviral vector system disclosed herein is a third-generation lentiviral vector system, one or more envelope plasmids do not encode an Env polypeptide. In some embodiments, one or more envelope plasmids of a lentiviral vector system encode one or more fusogens or a biologically active portions thereof. In some embodiments, a fusogen comprises a glycoprotein, e.g., protein G or portion thereof, and/or protein F or portions thereof. In some embodiments, one or more envelope plasmids encode paramyxovirus glycoprotein G (protein G) or a portion thereof and paramyxovirus fusion protein (protein F) or a portion thereof. In some embodiments, a fusogen comprises a chimeric protein. Various fusogens are contemplated as discussed further herein.

In some embodiments, a lentiviral vector system disclosed herein is a third-generation lentiviral vector system as disclosed herein. In some embodiments, a lentivirus system comprises one or more plasmids of a third-generation lentiviral vector system as disclosed herein. In some embodiments, a lentivirus system comprises one or more packaging plasmids, one or more envelope plasmids, one or more regulatory plasmids, and two or more transfer plasmids of a third-generation lentiviral vector system. In some embodiments, a lentivirus system comprises a packaging plasmid, a regulatory plasmid, an envelope plasmid and two or more transfer plasmids of a third-generation lentiviral vector system. In some embodiments, a lentivirus system comprises a packaging plasmid, a regulatory plasmid, two envelope plasmids and two or more transfer plasmids of a third-generation lentiviral vector system.

### D. Fourth Generation

Further iterations of lentiviral vector systems have been recently developed. For example, a fourth-generation lentiviral vector system having more than four plasmids has been reported to increases the number of recombination events required to generate replication-competent lentivirus (RCL). Such a fourth-generation lentiviral vector typically includes one or more plasmids whose expression is driven by a Tet-Off and/or Tat transactivator. A fourth-generation lentiviral vector system can include five plasmids, wherein one or more plasmid(s) is/are pTre-gag-pro, LTRHIV-vpr-pol, pCMV-VSVG, pMV-tet-off, and pTre-tat-ires-rev.

In some embodiments, a lentiviral vector system disclosed herein is a fourth-generation lentiviral vector system as disclosed herein. In some embodiments, a lentivirus system comprises one or more plasmids of a fourth-generation lentiviral vector system as disclosed herein. In some embodiments, a lentivirus system comprises one or more plasmids of a fourth-generation lentiviral vector system as disclosed herein, which include two or more transfer plasmids.

In some embodiments, when a lentiviral vector system disclosed herein is a fourth-generation lentiviral vector system, one or more envelope plasmids do not encode an Env polypeptide. In some embodiments, one or more envelope plasmids of a lentiviral vector system encode one or more fusogens or a biologically active portions thereof. In some embodiments, a fusogen comprises a glycoprotein, e.g., protein G or portion thereof, and/or protein F or portions thereof. In some embodiments, one or more envelope plasmids encode paramyxovirus glycoprotein G (protein G) or a portion thereof and paramyxovirus fusion protein (protein F) or a portion thereof. In some embodiments, a fusogen comprises a chimeric protein. Various fusogens are contemplated as discussed further herein.

In some embodiments, described herein is an engineered lentiviral vector system including: (i) one or more envelope plasmids, (ii) a packaging plasmid, and (iii) two or more transfer plasmids, wherein the two or more transfer plasmids include a first transfer plasmid that includes a first transgene and a second transfer plasmid that includes a second transgene, and wherein the first and the second transgene are different. In some embodiments, the first transfer plasmid and the second transfer plasmid are present at a ratio of between about 1:1 and 1:10 or between about 1:10 and 1:1. In some embodiments, the ratio of the first transfer plasmid and second transfer plasmid is about 1:1, about 1:2, about 1:3, about 1:4, about 1:5, about 1:6, about 1:7, about 1:8, about 1:9, about 1:10, about 2:1, about 3:1, about 4:1, about 5:1, about 6:1, about 7:1, about 8:1, about 9:1, about 10:1, about 2:3, about 3:2, about 2:5, about 3:5, about 5:2, or about 5:3.

In some embodiments, the first transgene encodes a first CAR and the second transgene encodes a second CAR, wherein the first CAR and the second CAR include different antigen binding domains that target different antigens expressed on a target cell. In some embodiments, the first CAR is a CD19 CAR and the second CAR is a CD22 CAR, the first CAR is a B cell maturation agent (BCMA) CAR and the second CAR is a G-protein coupled receptor family C group 5 member D (GPRC5D) CAR, the first CAR is a CD19 CAR and the second CAR is a CD20 CAR; the first CAR is a CD20 CAR and the second CAR is a CD22 CAR; or the first CAR is a CD19 CAR and the second CAR is a BCMA CAR; the first CAR is a CD20 CAR and the second CAR is a L1-CAM CAR, the first CAR is a L1-CAM CAR and the second CAR is a GD2 CAR; the first CAR is a EGFR CAR and the second CAR is a L1-CAM CAR; the first CAR is a EGFR CAR and the second CAR is a C-MET CAR; the first CAR is a EGFR CAR and the second CAR is a HER2 CAR, the first CAR is a C-MET CAR and the second CAR is a HER2 CAR; or the first CAR is a EGFR CAR and the second CAR is a ROR1 CAR.

In some embodiments, described herein is an engineered lentiviral vector system, wherein the first CAR and the second CAR are selected from the group including or consisting of a CD19 CAR, a CD22 CAR, B cell maturation agent (BCMA) CAR, a G-protein coupled receptor family C group 5 member D (GPRC5D) CAR, a CD5 CAR, a CD20 CAR, a CD23 CAR, a CD30 CAR, a CD33 CAR, CD38 CAR, a CD70 CAR, a CD123 CAR, a CD138 CAR, a Kappa CAR, Lambda CAR, a CD123 CAR, a LeY CAR, a NKG2D ligand CAR, a WT1 CAR, a GD2 CAR, a HER2 CAR, an EGFR CAR, an EGFRvIII CAR, a B7H3 CAR, a PSMA CAR, a PSCA CAR, a CAIX CAR, a CD171 CAR, a CEA CAR, a CSPG4 CAR, a EPHA2 CAR, a FAP CAR, a FRα CAR, a IL-13Rα CAR, a Mesothelin CAR, a MUC1 CAR, a MUC16 CAR, a ROR1 CAR, a C-Met CAR, a CD133 CAR, a Ep-CAM CAR, a GPC3 CAR, a HPV16-E6 CAR, a IL13Ra2 CAR, a MAGEA3 CAR, a MAGEA4 CAR, a MART1 CAR, a NY-ESO-1 CAR, a VEGFR2 CAR, a α-Folate receptor CAR, a CD24 CAR, a CD44v7/8 CAR, a EGP-2 CAR, a EGP-40 CAR, a erb-B2 CAR, a erb-B 2,3,4 CAR, a FBP CAR, a Fetal acethylcholine e receptor CAR, a GD2 CAR, a GD3 CAR, a HMW-MAA CAR, a IL-11Rα CAR, a KDR CAR, a Lewis Y CAR, a L1-cell adhesion molecule CAR, a MAGE-A1 CAR, a Oncofetal antigen (h5T4) CAR, a TAG-72 CAR, or a CD19/CD22 bispecific CAR.

In some embodiments, described herein is an engineered lentiviral vector system, the first CAR and the second CAR are selected from the group including or consisting of a CD19 CAR, a CD22 CAR, BCMA CAR, a GPRC5D CAR, a CD20 CAR, a CD19/CD22-bispecific CAR, a CD38 CAR, a CD123 CAR, or a CD138 CAR.

In some embodiments, described herein is an engineered lentiviral vector system, wherein the first transgene encodes a sequence-specific nuclease. In some embodiments, the sequence-specific nuclease is an RNA-guided nuclease, a transcription activator-like effector nuclease (TALEN), a meganuclease, a CRISPR-associated transposase, and a TnpB polypeptide, or a zinc-finger nuclease (ZFN). In some embodiments, the sequence-specific nuclease is selected from the group consisting of: Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, a CRISPR-associated transposase, and a TnpB polypeptide.

In some embodiments, described herein is an engineered lentiviral vector system, wherein the RNA-guided nuclease includes a Cas nuclease. In some embodiments, the Cas nuclease is a Type II or a Type V Cas protein. In some embodiments, the Cas nuclease is selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, and Mad7. In some embodiments, the Cas nuclease a Cas9 or a Cas12b. In some embodiments, the RNA-guided nuclease includes a Cas nuclease and a guide RNA (CRISPR-Cas combination). In some embodiments, the first transgene encodes a gRNA.

In some embodiments, first transgene encodes an antibody or portion thereof. In some embodiments, the first transgene encodes an antigen. In some embodiments, the first transgene encodes a therapeutic polypeptide. In some embodiments, the therapeutic polypeptide is useful for protein replacement therapy. In some embodiments, the first transgene encodes a CAR.

In some embodiments, the second transgene of the lentiviral vector system encodes a sequence-specific nuclease. In some embodiments, the sequence-specific nuclease is an RNA-guided nuclease, a transcription activator-like effector nuclease (TALEN), a meganuclease, a CRISPR-associated transposase, and a TnpB polypeptide, or a zinc-finger nuclease (ZFN). In some embodiments, the sequence-specific nuclease is selected from the group consisting of: Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, a CRISPR-associated transposase, and a TnpB polypeptide. In some embodiments the RNA-guided nuclease includes a Cas nuclease. In some embodiments, the Cas nuclease is a Type II or Type V Cas protein. In some embodiments, the Cas nuclease is selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, and Mad7. In some embodiments, the Cas nuclease a Cas9 or a Cas12b. In some embodiments, the RNA-guided nuclease includes a Cas nuclease and a guide RNA (CRISPR-Cas combination). In some embodiments, the second transgene encodes a gRNA.

In some embodiments, described herein is an engineered lentiviral vector system, wherein the second transgene encodes an antibody or portion thereof. In some embodiments, the second transgene encodes an antigen. In some embodiments, the second transgene encodes a therapeutic polypeptide. In some embodiments, the therapeutic polypeptide is useful for protein replacement therapy. In some embodiments, the second transgene encodes a CAR.

In some embodiments, described herein is an engineered lentiviral vector system, wherein the first transfer plasmid and/or the second transfer plasmid further includes a transgene encoding a tolerogenic factor. In some embodiments, the first transgene and/or the second transgene includes a biscistronic or a multicistronic expression cassette. In some embodiments, the tolerogenic factor is selected from the group including or consisting of CD47, a SIRPα engager, A20/TNFAIP3, B2M-HLA-E, CD16, CD16 Fc receptor, CD24, CD27, CD35, CD39, CD46, CD47, CD52, CD55, CD59, CD64, CD200, CCL21, CCL22, CTLA4-Ig, C1 inhibitor, CR1, DUX4, FASL, HLA-C, HLA-E, HLA-E heavy chain, HLA-F, HLA-G, H2-M3, IDO1, IL-10, IL15-RF, IL-35, IL-39, MANF, Mfge8, PD-L1, or Serpinb9. In some embodiments, the tolerogenic factor is CD47. In some embodiments, the first transfer plasmid includes a first tolerogenic factor and the second transfer plasmid includes a second tolerogenic factor, and the first and the second tolerogenic factors are different. In some embodiments, the first tolerogenic factor and the second tolerogenic factor are selected from the group including or consisting of CD47, a SIRPα engager, A20/TNFAIP3, B2M-HLA-E, CD16, CD16 Fc receptor, CD24, CD27, CD35, CD39, CD46, CD47, CD52, CD55, CD59, CD64, CD200, CCL21, CCL22, CTLA4-Ig, C1 inhibitor, CR1, DUX4, FASL, HLA-C, HLA-E, HLA-E heavy chain, HLA-F, HLA-G, H2-M3, IDO1, IL-10, IL15-RF, IL-35, IL-39, MANF, Mfge8, PD-L1, or Serpinb9. In some embodiments, first transfer plasmid includes a tolerogenic factor and the second transfer plasmid does not include a tolerogenic factor. In some embodiments, the first transfer plasmid further includes a transgene encoding CD47 and the second plasmid further includes a transgene encoding CD47.

In some embodiments, described herein is an engineered lentiviral vector system, the one or more envelope plasmids include a sequence encoding one or more fusogens. For example, the one or more fusogens can be any of the fusogens known in the art and/or disclosed herein. In some embodiments, the one or more fusogens include at least one fusogen that has a tropism for B cells, T cells, natural killer cells, islet cells, glial progenitor cells, neuronal cells, hematopoietic stem cells, cardiac cells, hepatocytes, stem cells, or induced pluripotent stem cells. In some embodiments, the one or more fusogens include at least one fusogen that has an endogenous tropism. In some embodiments, the one or more fusogens include at least one fusogen that has engineered tropism. In some embodiments, the one or more fusogens include one or more viral fusogens. In some embodiments, the one or more fusogens include at least one fusogen that is involved in attachment of a viral vector to a cell membrane. In some embodiments, the one or more fusogens include at least one fusogen that is involved in directing fusion of the lipid bilayer of a viral vector and a cell membrane. In some embodiments, the one or more fusogens include one or more paramyxovirus envelope proteins or biologically active portions thereof. In some embodiments, the one or more paramyxovirus envelope proteins or biologically active portions thereof includes a paramyxovirus glycoprotein (Protein G) or biologically active portion thereof. In some embodiments, the one or more paramyxovirus envelope proteins or biologically active portions thereof includes a paramyxovirus fusion protein (Protein F) or biologically active portion thereof. In some embodiments, the one or more fusogens include a Nipah virus fusion protein or a functional variant thereof. In some embodiments, the one or more fusogens include a Nipah virus F glycoprotein (NiV-F) or a biologically active portion thereof and a Nipah virus G glycoprotein (NiV-G) or a biologically active portion thereof. In some embodiments, the one or more fusogens include one or more chimeric proteins. In some embodiments, the one or more chimeric proteins include at least one chimeric protein that includes a paramyxovirus envelope protein or biologically active portion thereof. In some embodiments, the one or more chimeric proteins include at least one chimeric protein that includes a targeting agent that targets a target molecule. In some embodiments, the one or more chimeric proteins include at least one chimeric protein that includes (i) a paramyxovirus envelope protein or biologically active portion thereof and (ii) an targeting agent. In some embodiments, the NiV-G protein or biologically active portion thereof is fused to a targeting agent.

### II. Transgenes

The present disclosure provides lentiviral vectors and methods for producing viral vectors that can be used for numerous purposes. For example, as discussed above, in some embodiments, lentiviral vectors as described herein can include two or more transgenes. In some embodiments, a transfer plasmid, which includes one or more transgenes, is used in the production of a viral vector. In some embodiments, two or more transfer plasmid, which each include one or more transgenes, can be used in the production of a viral vector. Acceptable transgenes that can be included in such viral vectors can widely vary and can be used for a large number of purposes. The only meaningful characteristic of such a transgene is that it be a nucleic acid that can be packaged and delivered by a viral vector.

In some embodiments, a genome includes one or more transgenes that each encode a gene product. In some embodiments, a genome includes two or more, three or more, or four or more transgenes that each encode a gene product.

In some embodiments, a transfer plasmid includes one or more transgenes that each encode a gene product. In some embodiments, a transfer plasmid includes two or more, three or more, or four or more transgenes that each encode a gene product.

In some embodiments, a transgene encodes a gene product. A gene product can be an RNA or a polypeptide.

### Nucleic Acids

In some embodiments, the transgene includes a nucleic acid. For example, the transgene may encode RNA to enhance expression of an endogenous protein, or a siRNA or miRNA that inhibits protein expression of an endogenous protein. For example, the endogenous protein may modulate structure or function in the target cells. In some embodiments, the transgene may include a nucleic acid encoding an engineered protein that modulates structure or function in the target cells. In some embodiments, the transgene encodes a nucleic acid that targets a transcriptional activator that modulate structure or function in the target cells.

In some embodiments, the nucleic acid is, comprises, or consists of one or more natural nucleic acid residues. In some embodiments, the nucleic acid is, comprises, or consists of one or more nucleic acid analogs. In some embodiments, the nucleic acid has a nucleotide sequence that encodes a functional gene product such as an RNA or protein. In some embodiments, the nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, the nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, the nucleic acid is partly or wholly single stranded; in some embodiments, the nucleic acid is partly or wholly double stranded. In some embodiments the nucleic acid has a nucleotide sequence comprising at least one element that encodes, or is the complement of a sequence that encodes, a polypeptide. The nucleic acid may include variants, e.g., having an overall sequence identity with a reference nucleic acid of at least 85%, 86%, 87%, 88%, 89%, 90%, 91%, 92%, 93%, 94%, 95%, 96%, 97%, or 99%. In some embodiments, a variant nucleic acid does not share at least one characteristic sequence element with a reference nucleic acid. In some embodiments, a variant nucleic acid shares one or more of the biological activities of the reference nucleic acid. In some embodiments, a nucleic acid variant has a nucleic acid sequence that is identical to that of the reference but for a small number of sequence alterations at particular positions. In some embodiments, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. In some embodiments, a variant nucleic acid comprises about 10, about 9, about 8, about 7, about 6, about 5, about 4, about 3, about 2, or about 1 substituted residue as compared to a reference. In some embodiments, a variant nucleic acid comprises a very small number (e.g., fewer than about 5, about 4, about 3, about 2, or about 1) number of substituted, inserted, or deleted, functional residues that participate in a particular biological activity relative to the reference. In some embodiments, a variant nucleic acid comprises not more than about 15, about 12, about 9, about 3, or about 1 addition or deletion, and, in some embodiments, comprises no additions or deletions, as compared to the reference. In some embodiments, a variant nucleic acid comprises fewer than about 27, about 24, about 21, about 18, about 15, about 12, about 9, about 6, about 3, or fewer than about 9, about 6, about 3, or about 2 additions or deletions as compared to the reference.

In some embodiments, the transgene includes or encodes a nucleic acid, e.g., DNA, nDNA (nuclear DNA), mtDNA (mitochondrial DNA), protein coding DNA, gene, operon, chromosome, genome, transposon, retrotransposon, viral genome, intron, exon, modified DNA, mRNA (messenger RNA), tRNA (transfer RNA), modified RNA, microRNA, siRNA (small interfering RNA), tmRNA (transfer messenger RNA), rRNA (ribosomal RNA), mtRNA (mitochondrial RNA), snRNA (small nuclear RNA), small nucleolar RNA (snoRNA), SmY RNA (mRNA trans-splicing RNA), gRNA (guide RNA), TERC (telomerase RNA component), aRNA (antisense RNA), cis-NAT (Cis-natural antisense transcript), CRISPR RNA (crRNA), lncRNA (long noncoding RNA), piRNA (piwi-interacting RNA), shRNA (short hairpin RNA), tasiRNA (trans-acting siRNA), eRNA (enhancer RNA), satellite RNA, pcRNA (protein coding RNA), dsRNA (double stranded RNA), RNAi (interfering RNA), circRNA (circular RNA), reprogramming RNAs, aptamers, and any combination thereof. In some embodiments, the nucleic acid is a wild-type nucleic acid. In some embodiments, the nucleic acid is a mutant nucleic acid. In some embodiments the nucleic acid is a fusion or chimera of multiple nucleic acid sequences

In some embodiments, the nucleic acid encodes one or more (e.g. two or more) inhibitory RNA molecules directed against one or more RNA targets. An inhibitory RNA molecule can be, e.g., a miRNA or an shRNA. In some embodiments, the inhibitory molecule can be a precursor of a miRNA, such as for example, a Pri-miRNA or a Pre-miRNA, or a precursor of an shRNA. In some embodiments, the inhibitory molecule can be an artificially derived miRNA or shRNA. In other embodiments, the inhibitory RNA molecule can be a dsRNA (either transcribed or artificially introduced) that is processed into an siRNA or the siRNA itself. In some embodiments, the inhibitory RNA molecule can be a miRNA or shRNA that has a sequence that is not found in nature, or has at least one functional segment that is not found in nature, or has a combination of functional segments that are not found in nature. In illustrative embodiments, at least one or all of the inhibitory RNA molecules are miR-155. In some embodiments, a retroviral vector described herein encodes two or more inhibitory RNA molecules directed against one or more RNA targets. Two or more inhibitory RNA molecules, in some embodiments, can be directed against different targets. In other embodiments, the two or more inhibitory RNA molecules are directed against the same target. In some embodiments, the exogenous agent comprises a shRNA. A shRNA (short hairpin RNA) can comprise a double-stranded structure that is formed by a single self complementary RNA strand. shRNA constructs can comprise a nucleotide sequence identical to a portion, of either coding or non-coding sequence, of a target gene. RNA sequences with insertions, deletions, and single point mutations relative to the target sequence can also be used. Greater than 90% sequence identity, or even 100% sequence identity, between the inhibitory RNA and the portion of the target gene can be used. In certain embodiments, the length of the duplex-forming portion of an shRNA is at least 20, 2 1 or 22 nucleotides in length, e.g., corresponding in size to RNA products produced by Dicer-dependent cleavage. In certain embodiments, the shRNA construct is at least 25, 50, 100, 200, 300 or 400 bases in length. In certain embodiments, the shRNA construct is 400-800 bases in length. shRNA constructs are highly tolerant of variation in loop sequence and loop size. In embodiments, a retroviral vector that encodes an siRNA, an miRNA, an shRNA, or a ribozyme comprises one or more regulatory sequences, such as, for example, a strong constitutive pol III, e.g., human U6 snRNA promoter, the mouse U6 snRNA promoter, the human and mouse H1 RNA promoter and the human tRNA-val promoter, or a strong constitutive pol II promoter.

In some embodiments, the nucleic acid is operatively linked to a “positive target cell-specific regulatory element” (or positive TCSRE). In some embodiments, the positive TCSRE is a functional nucleic acid sequence. In some embodiments, the positive TCSRE contains a promoter or enhancer. In some embodiments, the TCSRE is a nucleic acid sequence that increases the level of an exogenous agent in a target cell.

In some embodiments, the nucleic acid is operatively linked to a “negative target cell-specific regulatory element” (or negative TCSRE). In some embodiments, the negative TCSRE is a functional nucleic acid sequence. In some embodiments, the negative TCSRE is a miRNA recognition site that causes degradation of inhibition of the viral vector in a non-target cell. In some embodiments, the nucleic acid is operatively linked to a “non-target cell-specific regulatory element” (or NTCSRE). In some embodiments, the NTCSRE comprises a nucleic acid sequence that decreases the level of an exogenous agent in a non-target cell compared to in a target cell. In some embodiments, the NTCSRE comprises a non-target cell-specific miRNA recognition sequence, non-target cell-specific protease recognition site, non-target cell-specific ubiquitin ligase site, non-target cell-specific transcriptional repression site, or non-target cell-specific epigenetic repression site. In some embodiments, the NTCSRE comprises a tissue-specific miRNA recognition sequence, tissue-specific protease recognition site, tissue-specific ubiquitin ligase site, tissue-specific transcriptional repression site, or tissue-specific epigenetic repression site. In some embodiments, the NTCSRE comprises a non-target cell-specific miRNA recognition sequence, non-target cell-specific protease recognition site, non-target cell-specific ubiquitin ligase site, non-target cell-specific transcriptional repression site, or non-target cell-specific epigenetic repression site. In some embodiments, the NTCSRE comprises a non-target cell-specific miRNA recognition sequence and the miRNA recognition sequence is able to be bound by one or more of miR3 1, miR363, or miR29c. In some embodiments, the NTCSRE is situated or encoded within a transcribed region encoding the exogenous agent, optionally wherein an RNA produced by the transcribed region comprises the miRNA recognition sequence within a UTR or coding region.

### Proteins

In some embodiments, the transgene encodes a protein. In some embodiments, the protein includes moieties other than amino acids (e.g., may be glycoproteins, proteoglycans, etc.) and/or may be otherwise processed or modified. In some embodiments, the protein includes more than one polypeptide chain, for example linked by one or more disulfide bonds or associated by other means.

In some embodiments, the protein contains L-amino acids, D-amino acids, or both and may contain any of a variety of amino acid modifications or analogs. In some embodiments, the protein contains natural amino acids, non-natural amino acids, synthetic amino acids, or combinations of any of the foregoing. In some embodiments, the protein is selected from antibodies, antibody fragments, biologically active portions thereof, and/or characteristic portions thereof.

In some embodiments, the protein is a wild-type protein. In some embodiments, the protein is a mutant protein. In some embodiments, the protein includes a polypeptide or its variants, e.g., having an overall sequence identity with a reference polypeptide of at least 85%, 86%, 87%, 88%, 89%, 90%, 91%, 92%, 93%, 94%, 95%, 96%, 97%, or 99%. In some embodiments, a variant polypeptide does not share at least one characteristic sequence element with a reference polypeptide. In some embodiments, a variant polypeptide shares one or more of the biological activities of the reference polypeptide. In some embodiments, a polypeptide variant has an amino acid sequence that is identical to that of the reference but for a small number of sequence alterations at particular positions. In some embodiments, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. In some embodiments, a variant polypeptide comprises about 10, about 9, about 8, about 7, about 6, about 5, about 4, about 3, about 2, or about 1 substituted residue as compared to a reference. In some embodiments, a variant polypeptide comprises a very small number (e.g., fewer than about 5, about 4, about 3, about 2, or about 1) number of substituted, inserted, or deleted, functional that participate in a particular biological activity relative to the reference. In some embodiments, a variant polypeptide comprises not more than about 5, about 4, about 3, about 2, or about 1 addition or deletion, and, in some embodiments, comprises no additions or deletions, as compared to the reference. In some embodiments, a variant polypeptide comprises fewer than about 25, about 20, about 19, about 18, about 17, about 16, about 15, about 14, about 13, about 10, about 9, about 8, about 7, about 6, and commonly fewer than about 5, about 4, about 3, or about 2 additions or deletions as compared to the reference.

In some embodiments, the protein includes a polypeptide, e.g., enzymes, structural polypeptides, signaling polypeptides, regulatory polypeptides, transport polypeptides, sensory polypeptides, motor polypeptides, defense polypeptides, storage polypeptides, transcription factors, antibodies, cytokines, hormones, catabolic polypeptides, anabolic polypeptides, proteolytic polypeptides, metabolic polypeptides, kinases, transferases, hydrolases, lyases, isomerases, ligases, enzyme modulator polypeptides, protein binding polypeptides, lipid binding polypeptides, membrane fusion polypeptides, cell differentiation polypeptides, epigenetic polypeptides, cell death polypeptides, nuclear transport polypeptides, nucleic acid binding polypeptides, reprogramming polypeptides, DNA editing polypeptides, DNA repair polypeptides, DNA recombination polypeptides, transposase polypeptides, DNA integration polypeptides, targeted endonucleases (e.g. Zinc-finger nucleases, transcription-activator-like nucleases (TALENs), cas9 and homologs thereof, cas12 and homologs thereof), recombinases, transposases, DNA polymerases, RNA polymerases, reverse transcriptases, and any combination of the foregoing.

In some embodiments, the protein targets a target protein in the cell for degradation. In some embodiments, the protein targets a target protein in the cell for degradation by localizing the protein to the proteasome. In some embodiments, the target protein is a wild-type protein. In some embodiments, the target protein is a mutant protein.

### A. Payload Gene

In some embodiments, the lentiviral vector comprises a transgene that is a payload gene encoding a payload agent. For example, the viral vector may comprise a nucleic acid that is or encodes an RNA to enhance expression of an endogenous protein, or a siRNA or miRNA that inhibits protein expression of an endogenous protein. For example, the endogenous protein may modulate structure or function in the target cells. In some embodiments, the viral vector may comprise a nucleic acid that is or encodes an engineered protein that modulates structure or function in the target cells. In some embodiments, the viral vector may comprise a nucleic acid that is or encodes a transcriptional activator that modulate structure or function in the target cells. The terms “transgene” and “payload gene” are used interchangeably herein unless context indicates otherwise.

In some embodiments, the viral vector herein comprises a nucleic acid, e.g., RNA or DNA. In some embodiments, the nucleic acid is, comprises, or consists of one or more natural nucleic acid residues. In some embodiments, the nucleic acid is, comprises, or consists of one or more nucleic acid analogs. In some embodiments, the nucleic acid has a nucleotide sequence that encodes a functional gene product such as an RNA or protein. In some embodiments, the nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, the nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, the nucleic acid is partly or wholly single stranded; in some embodiments, the nucleic acid is partly or wholly double stranded. In some embodiments the nucleic acid has a nucleotide sequence comprising at least one element that encodes, or is the complement of a sequence that encodes, a polypeptide.

In some embodiments, a transgene can be a multicistronic construct. The terms “multicistronic construct” and “multicistronic vector” are used interchangeably herein and refer to a recombinant DNA construct that is to be transcribed into a single mRNA molecule, wherein the single mRNA molecule encodes two or more gene products (e.g., two or more RNAs or polypeptides). The multicistronic construct is referred to as bicistronic construct if it encodes two gene products, and tricistronic construct if it encodes three gene products, and quadrocistronic construct if it encodes four gene products, and so on.

In some embodiments, the viral vector contains a nucleic acid that encodes a payload gene (also referred to as a “heterologous, recombinant, exogenous, or therapeutic gene.”).

In some embodiments, the payload gene encodes a protein that comprises a cytosolic protein, e.g., a protein that is produced in the recipient cell and localizes to the recipient cell cytoplasm. In some embodiments, the exogenous agent is a payload gene that encodes a protein that comprises a secreted protein, e.g., a protein that is produced and secreted by the recipient cell. In some embodiments, the transgene is a payload gene that encodes a protein that is a nuclear protein, e.g., a protein that is produced in the recipient cell and is imported to the nucleus of the recipient cell. In some embodiments, the transgene agent is a payload gene that encodes a protein that comprises an organellar protein (e.g., a mitochondrial protein), e.g., a protein that is produced in the recipient cell and is imported into an organelle (e.g., a mitochondrial) of the recipient cell.

In some embodiments, the payload gene encodes a protein that comprises a membrane protein. In some embodiments, the membrane protein comprises a chimeric antigen receptor (CAR), a T cell receptor, an integrin, an ion channel, a pore forming protein, a Toll-Like Receptor, an interleukin receptor, a cell adhesion protein, or a transport protein.

In some embodiments, the payload gene encodes a protein that is a nuclease for use in gene editing methods. In some embodiments, the nuclease is a zinc-finger nucleases (ZFNs), transcription-activator like effector nucleases (TALENs), or a CRISPR-associated protein-nuclease (Cas). In some embodiments, the Cas is Cas9 from Streptococcus pyogenes. In some embodiments, the Cas is a Cas12a (also known as cpf1) from a Prevotella or Francisella bacteria, or the Cas is a Cas12b from a Bacillus, optionally Bacillus hisashii. In some of any embodiments, the Cas is a Cas3, Cas13, CasMini, or any other Cas protein known in the art. See for example, Wang et al., Biosensors and Bioelectronics (165) 1: 2020, and Wu et al. Nature Reviews Chemistry (4) 441: 2020)

In some embodiments, the payload gene comprises a nucleic acid (i.e., a heterologous, recombinant, exogenous, or therapeutic gene) that encodes a cytosolic protein, e.g., a protein that is produced in the recipient cell and localizes to the recipient cell cytoplasm. In some embodiments, the payload gene comprises a nucleic acid that encodes a secreted protein, e.g., a protein that is produced and secreted by the recipient cell. In some embodiments, the payload gene comprises a nucleic acid that encodes a nuclear protein, e.g., a protein that is produced in the recipient cell and is imported to the nucleus of the recipient cell. In some embodiments, the payload gene comprises a nucleic acid that encodes an organellar protein (e.g., a mitochondrial protein), e.g., a protein that is produced in the recipient cell and is imported into an organelle (e.g., a mitochondrial) of the recipient cell.

In some embodiments, the payload gene comprises a nucleic acid (i.e., a heterologous, recombinant, exogenous, or therapeutic gene) that encodes a membrane protein. In some embodiments, the membrane protein comprises a nucleic acid that encodes a chimeric antigen receptor (CAR), a T cell receptor, an integrin, an ion channel, a pore forming protein, a Toll-Like Receptor, an interleukin receptor, a cell adhesion protein, or a transport protein. In some embodiments, delivery of the nuclease is by a provided vector encoding the nuclease (e.g. Cas).

In some embodiments, the payload gene is a globin gene. In some embodiments, the payload gene is ADA, IL2RG, JAK3, IL7R, HBB, F8, F9, WAS, CYBA, CYBB, NCF1, NCF2, NCF4, UROS, TCIRG1, CLCN7, MPL, ITGA2B, ITGB3, ITGB2, PKLR, SLC25, A38, RAG1, RAG2, FANCA, FANCC, FANCG, ABCD1, MAN2B1, AGA, LYST, CTNS, LAMP2, GLA, CTSA, GBA, GAA, IDS, IDUA, ISSD, ARSB, GALNS, GLB1, NEU1, GNPTA, SUMF1, SMPD1, NPC1, NPC2, CTSK, GNS, HGSNAT, NAGLU, SGSH, NAGA, GUSB, PSAP, LAL. In some embodiment, the payload gene can be a gene for delivery to a hematopoietic stem cell (HSC). Exemplary payload genes are described in WO2020102485, which is incorporated by reference.

For example, the payload gene can be, but is not limited to antisense ras, antisense myc, antisense raf, antisense erb, antisense src, antisense fins, antisense jun, antisense trk, antisense ret, antisense gsp, antisense hst, antisense bcl, antisense abl, Rb, CFTR, pi 6, p21, p27, p57, p73, C-CAM, APC, CTS-I, zacl, scFV ras, DCC, NF-I, NF-2, WT-I, MEN-I, MEN-II, BRCA1, VHL, MMAC1, FCC, MCC, BRCA2, IL-I, IL-2, IL-3, IL-4, IL-5, IL-6, IL-7, IL-8, IL-9, IL-10, IL-I1 IL-12, GM-CSF, G-CSF, thymidine kinase, mda7, fus-1, interferon α, interferon β, interferon γ, ADP, p53, ABLI, BLCl, BLC6, CBFA1, CBL, CSFIR, ERBA, ERBB, EBRB2, ETS1, ETS2, ETV6, FGR, FOX, FYN, HCR, HRAS, JUN, KRAS, LCK, LYN, MDM2, MLL, MYB, MYC, MYCL1, MYCN, NRAS, PIMI, PML, RET, SRC, TALI, TCL3, YES, MADH4, RBI, TP53, WT1, TNF, BDNF, CNTF, NGF, IGF, GMF, aFGF, bFGF, NT3, NT5, ApoAI, ApoAIV, ApoE, Rap1A, cytosine deaminase, Fab, ScFv, BRCA2, zacl, ATM, HIC-I, DPC-4, FHIT, PTEN, ING1, NOEY1, NOEY2, OVCA1, MADR2, 53BP2, IRF-I, Rb, zacl, DBCCR-I, rks-3, COX-I, TFPI, PGS, Dp, E2F, ras, myc, neu, raf, erb, fins, trk, ret, gsp, hst, abl, ElA, p300, VEGF, FGF, thrombospondin, BAI-I, GDAIF, or MCC. In further embodiments of the present invention, the payload gene is a gene encoding an ACP desaturase, an ACP hydroxylase, an ADP-glucose pyrophorylase, an ATPase, an alcohol dehydrogenase, an amylase, an amyloglucosidase, a catalase, a cellulase, a cyclooxygenase, a decarboxylase, a dextrinase, an esterase, a DNA polymerase, an RNA polymerase, a hyaluron synthase, a galactosidase, a glucanase, a glucose oxidase, a GTPase, a helicase, a hemicellulase, a hyaluronidase, an integrase, an invertase, an isomerase, a kinase, a lactase, a lipase, a lipoxygenase, a lyase, a lysozyme, a pectinesterase, a peroxidase, a phosphatase, a phospholipase, a phosphorylase, a polygalacturonase, a proteinase, a peptidase, a pullanase, a recombinase, a reverse transcriptase, a topoisomerase, a xylanase, a reporter gene, an interleukin, or a cytokine. In other embodiments of the present invention, the payload gene is a gene encoding carbamoyl synthetase I, ornithine transcarbamylase, arginosuccinate synthetase, arginosuccinate lyase, arginase, fumarylacetoacetate hydrolase, phenylalanine hydroxylase, alpha-1 antitrypsin, gmcose-6-phosphatase, low-density-lipoprotein receptor, porphobilinogen deaminase, factor VIII, factor IX, cystathione α-synthase, branched chain ketoacid decarboxylase, albumin, isovaleryl-CoA dehydrogenase, propionyl CoA carboxylase, methyl malonyl CoA mutase, glutaryl CoA dehydrogenase, insulin, beta.-glucosidase, pyruvate carboxylase, hepatic phosphorylase, phosphorylase kinase, glycine decarboxylase, H-protein, T-protein, Menkes disease copper-transporting ATPase, Wilson's disease copper-transporting ATPase, cytosine deaminase, hypoxanthine-guanine phosphoribosyltransferase. galactose-1-phosphate uridyltransferase, phenylalanine hydroxylase, glucocerebrosidase, sphingomyelinase, α-L-iduronidase, glucose-6-phosphate dehydrogenase, HSV thymidine kinase, or human thymidine kinase. Alternatively, the recombinant gene may encode growth hormone, prolactin, placental lactogen, luteinizing hormone, follicle-stimulating hormone, chorionic gonadotropin, thyroid-stimulating hormone, leptin, adrenocorticotropin, angiotensin I, angiotensin II, β-endorphin, β-melanocyte stimulating hormone, cholecystokinin, endothelin I, galanin, gastric inhibitory peptide, glucagon, insulin, lipotropins, neurophysins, somatostatin, calcitonin, calcitonin gene related peptide, β-calcitonin gene related peptide, hypercalcemia of malignancy factor, parathyroid hormone-related protein, parathyroid hormone-related protein, glucagon-like peptide, pancreastatin, pancreatic peptide, peptide YY, PHM, secretin, vasoactive intestinal peptide, oxytocin, vasopressin, vasotocin, enkephalinamide, metorphinamide, alpha melanocyte stimulating hormone, atrial natriuretic factor, amylin, amyloid P component, corticotropin releasing hormone, growth hormone releasing factor, luteinizing hormone-releasing hormone, neuropeptide Y, substance K, substance P, or thyrotropin releasing hormone.

### 1. Engineered Receptors

In some embodiments, the payload gene (i.e., transgene) encodes a payload agent that is an engineered receptor, such as antigen receptors including chimeric antigen receptors (CARs), and other antigen-binding receptors such as transgenic T cell receptors (TCRs). In some instances, a transgene encoding a Chimeric Antigen Receptor (CAR) can be delivered to a cell for expression. In some embodiments, the cell is a T cell, such as a primary T cell or a T cell differentiated from a pluripotent cell (e.g., iPSC). In some embodiments, the cell is a Natural Killer (NK) cell, such as a primary NK cell or an NK cell differentiated from a pluripotent cell (e.g., iPSC).

### (i) Chimeric Antigen Receptors (CARs)

In some embodiments, the payload agent is a chimeric antigen receptor (CAR) comprising an antigen binding domain. In some embodiments, the CAR is or comprises a first generation CAR comprising an antigen binding domain, a transmembrane domain, and signaling domain (e.g., one, two or three signaling domains). In some embodiments, the CAR comprises a third generation CAR comprising an antigen binding domain, a transmembrane domain, and at least three signaling domains. In some embodiments, a fourth generation CAR comprising an antigen binding domain, a transmembrane domain, three or four signaling domains, and a domain which upon successful signaling of the CAR induces expression of a cytokine gene. In some embodiments, the antigen binding domain is or comprises an scFv or Fab.

In some embodiments, described herein is an engineered lentiviral vector including: a first genome including a first transgene, and a second genome including a second transgene; wherein the first transgene encodes a first chimeric antigen receptor (CAR) and the second transgene encodes a second CAR, wherein the first CAR and the second CAR include different antigen binding domains that target different antigens expressed on a target cell. In some embodiments, described herein is an engineered lentiviral vector, wherein: the first CAR is a CD19 CAR and the second CAR is a CD22 CAR; the first CAR is a B cell maturation agent (BCMA) CAR and the second CAR is a G-protein coupled receptor family C group 5 member D (GPRC5D) CAR; the first CAR is a CD19 CAR and the second CAR is a CD20 CAR; the first CAR is a CD20 CAR and the second CAR is a CD22 CAR; or the first CAR is a CD19 CAR and the second CAR is a BCMA CAR; the first CAR is a CD20 CAR and the second CAR is a L1-CAM CAR; the first CAR is a L1-CAM CAR and the second CAR is a GD2 CAR; the first CAR is a EGFR CAR and the second CAR is a L1-CAM CAR; the first CAR is a EGFR CAR and the second CAR is a C-MET CAR; the first CAR is a EGFR CAR and the second CAR is a HER2 CAR; the first CAR is a C-MET CAR and the second CAR is a HER2 CAR; or the first CAR is a EGFR CAR and the second CAR is a ROR1 CAR.

In some embodiments, described herein is an engineered lentiviral vector, wherein the first CAR and the second CAR are selected from the group including or consisting of a CD19 CAR, a CD22 CAR, B cell maturation agent (BCMA) CAR, a G-protein coupled receptor family C group 5 member D (GPRC5D) CAR, a CD5 CAR, a CD20 CAR, a CD23 CAR, a CD30 CAR, a CD33 CAR, CD38 CAR, a CD70 CAR, a CD123 CAR, a CD138 CAR, a Kappa CAR, Lambda CAR, a CD123 CAR, a LeY CAR, a NKG2D ligand CAR, a WT1 CAR, a GD2 CAR, a HER2 CAR, an EGFR CAR, an EGFRvIII CAR, a B7H3 CAR, a PSMA CAR, a PSCA CAR, a CAIX CAR, a CD171 CAR, a CEA CAR, a CSPG4 CAR, a EPHA2 CAR, a FAP CAR, a FRα CAR, a IL-13Rα CAR, a Mesothelin CAR, a MUC1 CAR, a MUC16 CAR, a ROR1 CAR, a C-Met CAR, a CD133 CAR, a Ep-CAM CAR, a GPC3 CAR, a HPV16-E6 CAR, a IL13Ra2 CAR, a MAGEA3 CAR, a MAGEA4 CAR, a MART1 CAR, a NY-ESO-1 CAR, a VEGFR2 CAR, a α-Folate receptor CAR, a CD24 CAR, a CD44v7/8 CAR, a EGP-2 CAR, a EGP-40 CAR, a erb-B2 CAR, a erb-B 2,3,4 CAR, a FBP CAR, a Fetal acethylcholine e receptor CAR, a GD2 CAR, a GD3 CAR, a HMW-MAA CAR, a IL-11Rα CAR, a KDR CAR, a Lewis Y CAR, a L1-cell adhesion molecule CAR, a MAGE-A1 CAR, a Oncofetal antigen (h5T4) CAR, a TAG-72 CAR, or a CD19/CD22 bispecific CAR.

In some embodiments, described herein is an engineered lentiviral vector, the first CAR and the second CAR are selected from the group including or consisting of a CD19 CAR, a CD22 CAR, BCMA CAR, a GPRC5D CAR, a CD20 CAR, a CD19/CD22-bispecific CAR, a CD38 CAR, a CD123 CAR, or a CD138 CAR.

In some embodiments, the antigen binding domain targets an antigen characteristic of a cell type. In some embodiments, the antigen binding domain targets an antigen characteristic of a neoplastic cell. In some embodiments, the antigen characteristic of a neoplastic cell is selected from a cell surface receptor, an ion channel-linked receptor, an enzyme-linked receptor, a G protein-coupled receptor, receptor tyrosine kinase, tyrosine kinase associated receptor, receptor-like tyrosine phosphatase, receptor serine/threonine kinase, receptor guanylyl cyclase, histidine kinase associated receptor, Epidermal Growth Factor Receptors (EGFR) (including ErbB1/EGFR, ErbB2/HER2, ErbB3/HER3, and ErbB4/HER4), Fibroblast Growth Factor Receptors (FGFR) (including FGF1, FGF2, FGF3, FGF4, FGF5, FGF6, FGF7, FGF18, and FGF21) Vascular Endothelial Growth Factor Receptors (VEGFR) (including VEGF-A, VEGF-B, VEGF-C, VEGF-D, and PIGF), RET Receptor and the Eph Receptor Family (including EphA1, EphA2, EphA3, EphA4, EphA5, EphA6, EphA7, EphA8, EphA9, EphA10, EphB1, EphB2. EphB3, EphB4, and EphB6), CXCR1, CXCR2, CXCR3, CXCR4, CXCR6, CCR1, CCR2, CCR3, CCR4, CCR5, CCR6, CCR8, CFTR, CIC-1, CIC-2, CIC-4, CIC-5, CIC-7, CIC-Ka, CIC-Kb, Bestrophins, TMEM16A, GABA receptor, glycin receptor, ABC transporters, NAV1.1, NAV1.2, NAV1.3, NAV1.4, NAV1.5, NAV1.6, NAV1.7, NAV1.8, NAV1.9, sphingosin-1-phosphate receptor (S1P1R), NMDA channel, transmembrane protein, multispan transmembrane protein, T-cell receptor motifs; T-cell alpha chains; T-cell β chains; T-cell γ chains; T-cell δ chains; CCR7; CD3; CD4; CD5; CD7; CD8; CD11b; CD11c; CD16; CD19; CD20; CD21; CD22; CD25; CD28; CD34; CD35; CD40; CD45RA; CD45RO; CD52; CD56; CD62L; CD68; CD80; CD95; CD117; CD127; CD133; CD137 (4-1 BB); CD163; F4/80; IL-4Ra; Sea-1; CTLA-4; GITR; GARP; LAP; granzyme B; LFA-1; transferrin receptor; NKp46, perforin, CD4+; Th1; Th2; Th17; Th40; Th22; Th9; Tfh, Canonical Treg. FoxP3+; Tr1; Th3; Treg17; T_{RE}G; CDCP1, NT5E, EpCAM, CEA, gpA33, Mucins, TAG-72, Carbonic anhydrase IX, PSMA, Folate binding protein, Gangliosides (e.g., CD2, CD3, GM2), Lewis-γ², VEGF, VEGFR 1/2/3, αVβ3, α5β1, ErbB1/EGFR, ErbB1/HER2, ErB3, c-MET, IGF1R, EphA3, TRAIL-R1, TRAIL-R2, RANKL, FAP, Tenascin, PDL-1, BAFF, HDAC, ABL, FLT3, KIT, MET, RET, IL-1P, ALK, RANKL, mTOR, CTLA-4, IL-6, IL-6R, JAK3, BRAF, PTCH, Smoothened, PIGF, ANPEP, TIMP1, PLAUR, PTPRJ, LTBR, or ANTXR1, Folate receptor alpha (FRa), ERBB2 (Her2/neu), EphA2, IL-13Ra2, epidermal growth factor receptor (EGFR), Mesothelin, TSHR, CD19, CD123, CD22, CD30, CD171, CS-1, CLL-1, CD33, EGFRvIII, GD2, GD3, BCMA, MUC16 (CA125), L1CAM, LeY, MSLN, IL13Rα1, L1-CAM, Tn Ag, prostate specific membrane antigen (PSMA), ROR1, FLT3, FAP, TAG72, CD38, CD44v6, CEA, EPCAM, B7H3, KIT, interleukin-11 receptor a (IL-11Ra), PSCA, PRSS21, VEGFR2, LewisY, CD24, platelet-derived growth factor receptor-beta (PDGFR-beta), SSEA-4, CD20, MUC1, NCAM, Prostase, PAP, ELF2M, Ephrin B2, IGF-1 receptor, CAIX, LMP2, gplOO, bcr-abl, tyrosinase, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLACl, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6, E7, ETV6-AML, sperm protein 17, XAGE1, Tie 2, MAD-CT-1, MAD-CT-2, Major histocompatibility complex class I-related gene protein (MR1), urokinase-type plasminogen activator receptor (uPAR), Fos-related antigen 1, p53, p53 mutant, prostein, survivin, telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYPIB I, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, a neoantigen, CD133, CD15, CD184, CD24, CD56, CD26, CD29, CD44, HLA-A, HLA-B, HLA-C, (HLA-A,B,C) CD49f, CD151 CD340, CD200, tkrA, trkB, or trkC, or an antigenic fragment or antigenic portion thereof.

In some embodiments, the antigen binding domain targets an antigen characteristic of a T cell. In some embodiments, the antigen characteristic of a T cell is selected from a cell surface receptor, a membrane transport protein (e.g., an active or passive transport protein such as, for example, an ion channel protein, a pore-forming protein, etc.), a transmembrane receptor, a membrane enzyme, and/or a cell adhesion protein characteristic of a T cell. In some embodiments, an antigen characteristic of a T cell may be a G protein-coupled receptor, receptor tyrosine kinase, tyrosine kinase associated receptor, receptor-like tyrosine phosphatase, receptor serine/threonine kinase, receptor guanylyl cyclase, histidine kinase associated receptor, AKT1; AKT2; AKT3; ATF2; BCL10; CALM1; CD3D (CD3δ); CD3E (CD3ε); CD3G (CD3γ); CD4; CD8, CD28, CD45, CD80 (B7-1), CD86 (B7-2); CD247 (CD3ζ); CTLA4 (CD152); ELK1; ERK1 (MAPK3); ERK2; FOS; FYN; GRAP2 (GADS); GRB2; HLA-DRA; HLA-DRB1; HLA-DRB3; HLA-DRB4; HLA-DRB5; HRAS; IKBKA (CHUK); IKBKB; IKBKE; IKBKG (NEMO); IL2; ITPR1; ITK; JUN; KRAS2; LAT; LCK; MAP2K1 (MEK1); MAP2K2 (MEK2); MAP2K3 (MKK3); MAP2K4 (MKK4); MAP2K6 (MKK6); MAP2K7 (MKK7); MAP3K1 (MEKK1); MAP3K3; MAP3K4; MAP3K5; MAP3K8; MAP3K14 (NIK); MAPK8 (JNK1); MAPK9 (JNK2); MAPK10 (JNK3); MAPK11 (p38p); MAPK12 (p38γ); MAPK13 (p386); MAPK14 (p38a); NCK; NFAT1; NFAT2; NFKB1; NFKB2; NFKBIA; NRAS; PAK1; PAK2; PAK3; PAK4; PIK3C2B; PIK3C3 (VPS34); PIK3CA; PIK3CB; PIK3CD; PIK3R1; PKCA; PKCB; PKCM; PKCQ; PLCY1; PRF1 (Perforin), PTEN; RAC1; RAF1; RELA; SDF1; SHP2; SLP76; SOS; SRC; TBK1; TCRA; TEC; TRAF6; VAV1; VAV2; or ZAP70.

In some embodiments, the antigen binding domain targets an antigen characteristic of a disorder. In some embodiments, the antigen binding domain targets an antigen characteristic of an autoimmune or inflammatory disorder. In some embodiments, the autoimmune or inflammatory disorder is selected from chronic graft-vs-host disease (GVHD), lupus, arthritis, immune complex glomerulonephritis, goodpasture, uveitis, hepatitis, systemic sclerosis or scleroderma, type I diabetes, multiple sclerosis, cold agglutinin disease, Pemphigus vulgaris, Grave's disease, autoimmune hemolytic anemia, Hemophilia A, Primary Sjogren's Syndrome, thrombotic thrombocytopenia purrpura, neuromyelits optica, Evan's syndrome, IgM mediated neuropathy, cyroglobulinemia, dermatomyositis, idiopathic thrombocytopenia, ankylosing spondylitis, bullous pemphigoid, acquired angioedema, chronic urticarial, antiphospholipid demyelinating polyneuropathy, and autoimmune thrombocytopenia or neutropenia or pure red cell aplasias, while exemplary non-limiting examples of alloimmune diseases include allosensitization (see, for example, Blazar et al., 2015, Am. J. Transplant, 15(4):931-41) or xenosensitization from hematopoietic or solid organ transplantation, blood transfusions, pregnancy with fetal allosensitization, neonatal alloimmune thrombocytopenia, hemolytic disease of the newborn, sensitization to foreign antigens such as can occur with replacement of inherited or acquired deficiency disorders treated with enzyme or protein replacement therapy, blood products, and gene therapy. In some embodiments, the antigen characteristic of an an autoimmune or inflammatory disorder is selected from a cell surface receptor, an ion channel-linked receptor, an enzyme-linked receptor, a G protein-coupled receptor, receptor tyrosine kinase, tyrosine kinase associated receptor, receptor-like tyrosine phosphatase, receptor serine/threonine kinase, receptor guanylyl cyclase, or histidine kinase associated receptor. In some embodiments, a CAR antigen binding domain binds to a ligand expressed on B cells, plasma cells, plasmablasts, CD10, CD19, CD20, CD22, CD24, CD27, CD38, CD45R, CD138, CD319, BCMA, CD28, TNF, interferon receptors, GM-CSF, ZAP-70, LFA-1, CD3 gamma, CD5 or CD2. See US 2003/0077249; WO 2017/058753; WO 2017/058850, the contents of which are herein incorporated by reference.

In some embodiments, the antigen binding domain targets an antigen characteristic of senescent cells, e.g., urokinase-type plasminogen activator receptor (uPAR). In some embodiments, the CAR may be used for treatment or prophylaxis of disorders characterized by the aberrant accumulation of senescent cells, e.g., liver and lung fibrosis, atherosclerosis, diabetes and osteoarthritis.

In some embodiments, the antigen binding domain targets an antigen characteristic of an infectious disease. In some embodiments, wherein the infectious disease is selected from HIV, hepatitis B virus, hepatitis C virus, Human herpes virus, Human herpes virus 8 (HHV-8, Kaposi sarcoma-associated herpes virus (KSHV)), Human T-lymphotrophic virus-1 (HTLV-1), Merkel cell polyomavirus (MCV), Simian virus 40 (SV40), Eptstein-Barr virus, CMV, human papillomavirus. In some embodiments, the antigen characteristic of an infectious disease is selected from a cell surface receptor, an ion channel-linked receptor, an enzyme-linked receptor, a G protein-coupled receptor, receptor tyrosine kinase, tyrosine kinase associated receptor, receptor-like tyrosine phosphatase, receptor serine/threonine kinase, receptor guanylyl cyclase, histidine kinase associated receptor, HIV Env, gp120, or CD4-induced epitope on HIV-1 Env.

In some embodiments, the CAR transmembrane domain comprises at least a transmembrane region of the alpha, beta or zeta chain of a T cell receptor, CD28, CD3 epsilon, CD45, CD4, CD5, CD8, CD9, CD16, CD22, CD33, CD37, CD64, CD80, CD86, CD134, CD137, CD154, or functional variant thereof. In some embodiments, the transmembrane domain comprises at least a transmembrane region(s) of CD8α, CD8β, 4-1BB/CD137, CD28, CD34, CD4, FcεRIγ, CD16, OX40/CD134, CD3ζ, CD3ε, CD3γ, CD3δ, TCRα, TCRβ, TCRζ, CD32, CD64, CD64, CD45, CD5, CD9, CD22, CD37, CD80, CD86, CD40, CD40L/CD154, VEGFR2, FAS, and FGFR2B, or functional variant thereof.

In some embodiments, the CAR comprises at least one signaling domain selected from one or more of B7-1/CD80; B7-2/CD86; B7-H1/PD-L1; B7-H2; B7-H3; B7-H4; B7-H6; B7-H7; BTLA/CD272; CD28; CTLA-4; Gi24/VISTA/B7-H5; ICOS/CD278; PD-1; PD-L2/B7-DC; PDCD6); 4-1BB/TNFSF9/CD137; 4-1BB Ligand/TNFSF9; BAFF/BLyS/TNFSF13B; BAFF R/TNFRSF13C; CD27/TNFRSF7; CD27 Ligand/TNFSF7; CD30/TNFRSF8; CD30 Ligand/TNFSF8; CD40/TNFRSF5; CD40/TNFSF5; CD40 Ligand/TNFSF5; DR3/TNFRSF25; GITR/TNFRSF18; GITR Ligand/TNFSF18; HVEM/TNFRSF14; LIGHT/TNFSF14; Lymphotoxin-alpha/TNF-beta; OX40/TNFRSF4; OX40 Ligand/TNFSF4; RELT/TNFRSF19L; TACI/TNFRSF13B; TL1A/TNFSF15; TNF-alpha; TNF RII/TNFRSF1B); 2B4/CD244/SLAMF4; BLAME/SLAMF8; CD2; CD2F-10/SLAMF9; CD48/SLAMF2; CD58/LFA-3; CD84/SLAMF5; CD229/SLAMF3; CRACC/SLAMF7; NTB-A/SLAMF6; SLAM/CD150); CD2; CD7; CD53; CD82/Kai-1; CD90/Thy1; CD96; CD160; CD200; CD300a/LMIR1; HLA Class I; HLA-DR; Ikaros; Integrin alpha 4/CD49d; Integrin alpha 4 beta 1; Integrin alpha 4 beta 7/LPAM-1; LAG-3; TCL1A; TCL1B; CRTAM; DAP12; Dectin-1/CLEC7A; DPPIV/CD26; EphB6; TIM-1/KIM-1/HAVCR; TIM-4; TSLP; TSLP R; lymphocyte function associated antigen-1 (LFA-1); NKG2C, a CD3 zeta domain, an immunoreceptor tyrosine-based activation motif (ITAM), CD27, CD28, 4-1BB, CD134/OX40, CD30, CD40, PD-1, ICOS, lymphocyte function-associated antigen-1 (LFA-1), CD2, CD7, LIGHT, NKG2C, B7-H3, a ligand that specifically binds with CD83, or functional fragment thereof.

In some embodiments, the CAR comprises a CD3 zeta domain or an immunoreceptor tyrosine-based activation motif (ITAM), or functional variant thereof. In some embodiments, the CAR comprises (i) a CD3 zeta domain, or an immunoreceptor tyrosine-based activation motif (ITAM), or functional variant thereof, and (ii) a CD28 domain, or a 4-1BB domain, or functional variant thereof. In some embodiments, the CAR comprises a (i) a CD3 zeta domain, or an immunoreceptor tyrosine-based activation motif (ITAM), or functional variant thereof, (ii) a CD28 domain or functional variant thereof, and (iii) a 4-1BB domain, or a CD134 domain, or functional variant thereof. In some embodiments, the CAR comprises (i) a CD3 zeta domain, or an immunoreceptor tyrosine-based activation motif (ITAM), or functional variant thereof, (ii) a CD28 domain, or a 4-1BB domain, or functional variant thereof, and/or (iii) a 4-1BB domain, or a CD134 domain, or functional variant thereof. In some embodiments, the CAR comprises a (i) a CD3 zeta domain, or an immunoreceptor tyrosine-based activation motif (ITAM), or functional variant thereof, (ii) a CD28 domain or functional variant thereof, (iii) a 4-1BB domain, or a CD134 domain, or functional variant thereof, and (iv) a cytokine or costimulatory ligand transgene.

In some embodiments, the CAR further comprises one or more spacers, e.g., wherein the spacer is a first spacer between the antigen binding domain and the transmembrane domain. In some embodiments, the first spacer includes at least a portion of an immunoglobulin constant region or variant or modified version thereof. In some embodiments, the spacer is a second spacer between the transmembrane domain and a signaling domain. In some embodiments, the second spacer is an oligopeptide, e.g., wherein the oligopeptide comprises glycine-serine doublets.

In some embodiments, the payload agent is or comprises a CAR, e.g., a first generation CAR or a nucleic acid encoding a first generation CAR. In some embodiments, a first generation CAR comprises an antigen binding domain, a transmembrane domain, and signaling domain. In some embodiments a signaling domain mediates downstream signaling during T cell activation.

In some embodiments, the payload agent is or comprises a second generation CAR or a nucleic acid encoding a second generation CAR. In some embodiments a second generation CAR comprises an antigen binding domain, a transmembrane domain, and two signaling domains. In some embodiments a signaling domain mediates downstream signaling during T cell activation. In some embodiments a signaling domain is a costimulatory domain. In some embodiments, a costimulatory domain enhances cytokine production, CAR T cell proliferation, and or CAR T cell persistence during T cell activation.

In some embodiments, the payload agent is or comprises a third generation CAR or a nucleic acid encoding a third generation CAR. In some embodiments, a third generation CAR comprises an antigen binding domain, a transmembrane domain, and at least three signaling domains. In some embodiments a signaling domain mediates downstream signaling during T cell activation. In some embodiments a signaling domain is a costimulatory domain. In some embodiments, a costimulatory domain enhances cytokine production, CAR T cell proliferation, and or CAR T cell persistence during T cell activation. In some embodiments, a third generation CAR comprises at least two costimulatory domains. In some embodiments, the at least two costimulatory domains are not the same.

In some embodiments, the payload agent is or comprises a fourth generation CAR or a nucleic acid encoding a fourth generation CAR. In some embodiments a fourth generation CAR comprises an antigen binding domain, a transmembrane domain, and at least two, three, or four signaling domains. In some embodiments a signaling domain mediates downstream signaling during T cell activation. In some embodiments a signaling domain is a costimulatory domain. In some embodiments, a costimulatory domain enhances cytokine production, CAR T cell proliferation, and or CAR T cell persistence during T cell activation.

In some embodiments, a first, second, third, or fourth generation CAR further comprises a domain which upon successful signaling of the CAR induces expression of a cytokine gene. In some embodiments, a cytokine gene is endogenous or exogenous to a target cell comprising a CAR which comprises a domain which upon successful signaling of the CAR induces expression of a cytokine gene. In some embodiments a cytokine gene encodes a pro-inflammatory cytokine. In some embodiments a cytokine gene encodes IL-1, IL-2, IL-9, IL-12, IL-18, TNF, or IFN-gamma, or functional fragment thereof. In some embodiments a domain which upon successful signaling of the CAR induces expression of a cytokine gene is or comprises a transcription factor or functional domain or fragment thereof. In some embodiments a domain which upon successful signaling of the CAR induces expression of a cytokine gene is or comprises a transcription factor or functional domain or fragment thereof. In some embodiments a transcription factor or functional domain or fragment thereof is or comprises a nuclear factor of activated T cells (NFAT), an NF-kB, or functional domain or fragment thereof. See, e.g., Zhang. C. et al., Engineering CAR-T cells. Biomarker Research. 5:22 (2017); WO 2016126608; Sha, H. et al. Chimaeric antigen receptor T-cell therapy for tumour immunotherapy. Bioscience Reports Jan. 27, 2017, 37 (1).

In some embodiments, a CAR antigen binding domain is or comprises an antibody or antigen-binding portion thereof. In some embodiments, a CAR antigen binding domain is or comprises an scFv or Fab. In some embodiments a CAR antigen binding domain comprises an scFv or Fab fragment of a BCMA antibody, T-cell alpha chain antibody; T-cell β chain antibody; T-cell γ chain antibody; T-cell δ chain antibody; CCR7 antibody; CD3 antibody; CD4 antibody; CD5 antibody; CD7 antibody; CD8 antibody; CD11b antibody; CD11c antibody; CD16 antibody; CD19 antibody; CD20 antibody; CD21 antibody; CD22 antibody; CD25 antibody; CD28 antibody; CD34 antibody; CD35 antibody; CD40 antibody; CD45RA antibody; CD45RO antibody; CD52 antibody; CD56 antibody; CD62L antibody; CD68 antibody; CD80 antibody; CD95 antibody; CD117 antibody; CD127 antibody; CD133 antibody; CD137 (4-1 BB) antibody; CD163 antibody; F4/80 antibody; IL-4Rα antibody; Sca-1 antibody; CTLA-4 antibody; GITR antibody; GARP antibody; GPRC5D antibody; LAP antibody; granzyme B antibody; LFA-1 antibody; MIR1 antibody; uPAR antibody; or transferrin receptor antibody.

In some embodiments, an antigen binding domain binds to a cell surface antigen of a cell. In some embodiments, a cell surface antigen is characteristic of one type of cell. In some embodiments, a cell surface antigen is characteristic of more than one type of cell.

In some embodiments, a CAR antigen binding domain binds a cell surface antigen characteristic of a T cell. In some embodiments, an antigen characteristic of a T cell may be a cell surface receptor, a membrane transport protein (e.g., an active or passive transport protein such as, for example, an ion channel protein, a pore-forming protein, etc.), a transmembrane receptor, a membrane enzyme, and/or a cell adhesion protein characteristic of a T cell. In some embodiments, an antigen characteristic of a T cell may be a G protein-coupled receptor, receptor tyrosine kinase, tyrosine kinase associated receptor, receptor-like tyrosine phosphatase, receptor serine/threonine kinase, receptor guanylyl cyclase, or histidine kinase associated receptor.

In some embodiments, a CAR comprises a signaling domain which is a costimulatory domain. In some embodiments a CAR comprises a second costimulatory domain. In some embodiments a CAR comprises at least two costimulatory domains. In some embodiments a CAR comprises at least three costimulatory domains. In some embodiments a CAR comprises a costimulatory domain selected from one or more of CD27, CD28, 4-1BB, CD134/OX40, CD30, CD40, PD-1, ICOS, lymphocyte function-associated antigen-1 (LFA-1), CD2, CD7, LIGHT, NKG2C, B7-H3, a ligand that specifically binds with CD83.

In some embodiments, the CAR comprises a CD3 zeta domain or an immunoreceptor tyrosine-based activation motif (ITAM), or functional variant thereof. In some embodiments, the CAR comprises (i) a CD3 zeta domain, or an immunoreceptor tyrosine-based activation motif (ITAM), or functional variant thereof, and (ii) a CD28 domain, or a 4-1BB domain, or functional variant thereof. In some embodiments, the CAR comprises a (i) a CD3 zeta domain, or an immunoreceptor tyrosine-based activation motif (ITAM), or functional variant thereof, (ii) a CD28 domain or functional variant thereof, and (iii) a 4-1BB domain, or a CD134 domain, or functional variant thereof. In some embodiments, the CAR comprises (i) a CD3 zeta domain, or an immunoreceptor tyrosine-based activation motif (ITAM), or functional variant thereof, (ii) a CD28 domain, or a 4-1BB domain, or functional variant thereof, and/or (iii) a 4-1BB domain, or a CD134 domain, or functional variant thereof. In some embodiments, the CAR comprises a (i) a CD3 zeta domain, or an immunoreceptor tyrosine-based activation motif (ITAM), or functional variant thereof, (ii) a CD28 domain or functional variant thereof, (iii) a 4-1BB domain, or a CD134 domain, or functional variant thereof, and (iv) a cytokine or costimulatory ligand transgene.

In certain embodiments, the intracellular signaling domain comprises a CD28 transmembrane and signaling domain linked to a CD3 (e.g., CD3-zeta) intracellular domain. In some embodiments, the intracellular signaling domain comprises a chimeric CD28 and CD137 (4-1BB, TNFRSF9) co-stimulatory domains, linked to a CD3 zeta intracellular domain.

In some embodiments, the CAR encompasses one or more, e.g., two or more, costimulatory domains and an activation domain, e.g., primary activation domain, in the cytoplasmic portion. Exemplary CARs include intracellular components of CD3-zeta, CD28, and 4-1BB.

In some embodiments the intracellular signaling domain includes intracellular components of a 4-1BB signaling domain and a CD3-zeta signaling domain. In some embodiments, the intracellular signaling domain includes intracellular components of a CD28 signaling domain and a CD3zeta signaling domain.

In some embodiments, the CAR comprises an extracellular antigen binding domain (e.g., antibody or antibody fragment, such as an scFv) that binds to an antigen (e.g. tumor antigen), a spacer (e.g. containing a hinge domain, such as any as described herein), a transmembrane domain (e.g. any as described herein), and an intracellular signaling domain (e.g. any intracellular signaling domain, such as a primary signaling domain or costimulatory signaling domain as described herein). In some embodiments, the intracellular signaling domain is or includes a primary cytoplasmic signaling domain. In some embodiments, the intracellular signaling domain additionally includes an intracellular signaling domain of a costimulatory molecule (e.g., a costimulatory domain).

In some embodiments, the CAR contains one or more domains that combine an antigen- or ligand-binding domain (e.g., antibody or antibody fragment) that provides specificity for a desired antigen (e.g., tumor antigen) with intracellular signaling domains. In some embodiments, the intracellular signaling domain is a stimulating or an activating intracellular domain portion, such as a T cell stimulating or activating domain, providing a primary activation signal or a primary signal. In some embodiments, the intracellular signaling domain contains or additionally contains a costimulatory signaling domain to facilitate effector functions. In some embodiments, chimeric receptors when genetically engineered into immune cells can modulate T cell activity, and, in some cases, can modulate T cell differentiation or homeostasis, thereby resulting in genetically engineered cells with improved longevity, survival and/or persistence in vivo, such as for use in adoptive cell therapy methods.

Exemplary antigen receptors, including CARs, and methods for engineering and introducing such receptors into cells, include those described, for example, in WO200014257, WO2013126726, WO2012/129514, WO2014031687, WO2013/166321, WO2013/071154, WO2013/123061, U.S. patent app. Pub. Nos. US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent app. No. EP2537416, and/or those described by Sadelain et al., Cancer Discov. 2013 April; 3(4): 388-398; Davila et al. (2013) PLoS ONE 8(4): e61338; Turtle et al., Curr. Opin. Immunol., 2012 October; 24(5): 633-39; Wu et al., Cancer, 2012 Mar. 18(2): 160-75. In some aspects, the antigen receptors include a CAR as described in U.S. Pat. No. 7,446,190, and those described in WO/2014055668. Examples of the CARs include CARs as disclosed in any of the aforementioned publications, such as WO2014031687, U.S. Pat. Nos. 8,339,645, 7,446,179, US 2013/0149337, U.S. Pat. Nos. 7,446,190, 8,389,282, Kochenderfer et al., (2013) Nature Reviews Clinical Oncology, 10, 267-276; Wang et al. (2012) J. Immunother. 35(9): 689-701; and Brentjens et al., Sci Transl Med. 2013 5(177). See also WO2014031687, U.S. Pat. Nos. 8,339,645, 7,446,179, US 2013/0149337, U.S. Pat. Nos. 7,446,190, and 8,389,282. The recombinant receptors, such as CARs, generally include an extracellular antigen binding domain, such as a portion of an antibody molecule, generally a variable heavy (VH) chain region and/or variable light (VL) chain region of the antibody, e.g., an scFv antibody fragment. In some embodiments, the antigen binding domain of the CAR molecule comprises an antibody, an antibody fragment, an scFv, a Fv, a Fab, a (Fab′)₂, a single domain antibody (SdAb), a VH or VL domain, or a camelid VHH domain.

In some embodiments, a CAR antigen binding domain is or comprises an antibody or antigen-binding portion thereof. In some embodiments, a CAR antigen binding domain is or comprises an scFv or Fab. In some embodiments, a CAR antigen binding domain comprises an scFv or Fab fragment of a CD19 antibody; CD22 antibody; CD22 antibody; GPRC5D antibody; T-cell alpha chain antibody; T-cell P chain antibody; T-cell γ chain antibody; T-cell δ chain antibody; CCR7 antibody; CD3 antibody; CD4 antibody; CD5 antibody; CD7 antibody; CD8 antibody; CD11b antibody; CD11c antibody; CD16 antibody; CD20 antibody; CD21 antibody; CD25 antibody; CD28 antibody; CD34 antibody; CD35 antibody; CD40 antibody; CD45RA antibody; CD45RO antibody; CD52 antibody; CD56 antibody; CD62L antibody; CD68 antibody; CD80 antibody; CD95 antibody; CD117 antibody; CD127 antibody; CD133 antibody; CD137 (4-1 BB) antibody; CD163 antibody; F4/80 antibody; IL-4Rα antibody; Sca-1 antibody; CTLA-4 antibody; GITR antibody GARP antibody; LAP antibody; granzyme B antibody; LFA-1 antibody; MR1 antibody; uPAR antibody; or transferrin receptor antibody.

In some embodiments, a CAR comprises a signaling domain which is a costimulatory domain. In some embodiments, a CAR comprises a second costimulatory domain. In some embodiments, a CAR comprises at least two costimulatory domains. In some embodiments, a CAR comprises at least three costimulatory domains. In some embodiments, a CAR comprises a costimulatory domain selected from one or more of CD27, CD28, 4-1BB, CD134/OX40, CD30, CD40, PD-1, ICOS, lymphocyte function-associated antigen-1 (LFA-1), CD2, CD7, LIGHT, NKG2C, B7-H3, a ligand that specifically binds with CD83. In some embodiments, if a CAR comprises two or more costimulatory domains, two costimulatory domains are different. In some embodiments, if a CAR comprises two or more costimulatory domains, two costimulatory domains are the same.

In addition to the CARs described herein, various chimeric antigen receptors and nucleotide sequences encoding the same are known in the art and would be suitable for fusosomal delivery and reprogramming of target cells in vivo and in vitro as described herein. See, e.g., WO2013040557; WO2012079000; WO2016030414; Smith T, et al., Nature Nanotechnology. 2017. DOI: 10.1038/NNANO.2017.57, the disclosures of which are herein incorporated by reference.

In some embodiments, the antigen targeted by the receptor is a polypeptide. In some embodiments, it is a carbohydrate or other molecule. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

In some embodiments, the antigen targeted by the receptor includes antigens associated with a B cell malignancy, such as any of a number of known B cell markers. In some embodiments, the antigen targeted by the receptor is CD20, CD19, CD22, ROR1, CD45, CD47, CD21, CD5, CD33, Igkappa, Iglambda, CD79a, CD79b or CD30.

In some embodiments, the CAR binds to CD19. In some embodiments, the CAR binds to CD22. In some embodiments, the CAR binds to CD19 and CD22. In some embodiments, the CAR is selected from the group consisting of a first generation CAR, a second generation CAR, a third generation CAR, and a fourth generation CAR. In some embodiments, the CAR includes a single binding domain that binds to a single target antigen. In some embodiments, the CAR includes a single binding domain that binds to more than one target antigen, e.g., 2, 3, or more target antigens. In some embodiments, the CAR includes two binding domains such that each binding domain binds to a different target antigens. In some embodiments, the CAR includes two binding domains such that each binding domain binds to the same target antigen. Detailed descriptions of exemplary CARs including CD19-specific, CD22-specific and CD19/CD22-bispecific CARs can be found in WO2012/079000, WO2016/149578 and WO2020/014482, the disclosures including the sequence listings and figures are incorporated herein by reference in their entirety.

In some embodiments, the chimeric antigen receptor includes an extracellular portion containing an antibody or antibody fragment. In some aspects, the chimeric antigen receptor includes an extracellular portion containing the antibody or fragment and an intracellular signaling domain. In some embodiments, the antibody or fragment includes an scFv.

In some embodiments, the antigen targeted by the antigen-binding domain is CD19. In some aspects, the antigen-binding domain of the recombinant receptor, e.g., CAR, and the antigen-binding domain binds, such as specifically binds or specifically recognizes, a CD19, such as a human CD19. In some embodiments, the scFv contains a VH and a VL derived from an antibody or an antibody fragment specific to CD19. In some embodiments, the antibody or antibody fragment that binds CD19 is a mouse derived antibody such as FMC63 and SJ25C1. In some embodiments, the antibody or antibody fragment is a human antibody, e.g., as described in U.S. Patent Publication No. US 2016/0152723.

In some embodiments, the antigen is CD19. In some embodiments, the scFv contains a VH and a VL derived from an antibody or an antibody fragment specific to CD 19. In some embodiments, the antibody or antibody fragment that binds CD 19 is a mouse derived antibody such as FMC63 and SJ25CJ. In some embodiments, the antibody or antibody fragment is a human antibody, e.g., as described in U.S. Patent Publication No. US 2016/0152723.

In some embodiments, the scFv is derived from FMC63. FMC63 generally refers to a mouse monoclonal IgGl antibody raised against Naim-1 and -16 cells expressing CD19 of human origin (Fing, N. R., et al. (1987). Leucocyte typing III. 302).

In some embodiments, the antibody portion of the recombinant receptor, e.g., CAR, further includes spacer between the transmembrane domain and extracellular antigen binding domain. In some embodiments, the spacer includes at least a portion of an immunoglobulin constant region, such as a hinge region, e.g., an IgG4 hinge region, and/or a CH1/CL and/or Fc region. In some embodiments, the constant region or portion is of a human IgG, such as IgG4 or IgG1. In some aspects, the portion of the constant region serves as a spacer region between the antigen-recognition component, e.g., scFv, and transmembrane domain. The spacer can be of a length that provides for increased responsiveness of the cell following antigen binding, as compared to in the absence of the spacer. Exemplary spacers include, but are not limited to, those described in Hudecek et al. (2013) Clin. Cancer Res., 19:3153, WO2014031687, U.S. Pat. No. 8,822,647 or published app. No. US 2014/0271635. In some embodiments, the constant region or portion is of a human IgG, such as IgG4 or IgG1.

In some embodiments, the antigen receptor comprises an intracellular domain linked directly or indirectly to the extracellular domain. In some embodiments, the chimeric antigen receptor includes a transmembrane domain linking the extracellular domain and the intracellular signaling domain. In some embodiments, the intracellular signaling domain comprises an ITAM. For example, in some aspects, the antigen recognition domain (e.g. extracellular domain) generally is linked to one or more intracellular signaling components, such as signaling components that mimic activation through an antigen receptor complex, such as a TCR complex, in the case of a CAR, and/or signal via another cell surface receptor. In some embodiments, the chimeric receptor comprises a transmembrane domain linked or fused between the extracellular domain (e.g. scFv) and intracellular signaling domain. Thus, in some embodiments, the antigen-binding component (e.g., antibody) is linked to one or more transmembrane and intracellular signaling domains.

In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

In some embodiments, the CAR transmembrane domain comprises at least a transmembrane region of the alpha, beta or zeta chain of a T cell receptor, CD28, CD3 epsilon, CD45, CD4, CD5, CD8, CD9, CD16, CD22, CD33, CD37, CD64, CD80, CD86, CD134, CD137, CD154, or functional variant thereof. In some embodiments, the transmembrane domain comprises at least a transmembrane region(s) of CD8α, CD8β, 4-1BB/CD137, CD28, CD34, CD4, FcεRIγ, CD16, OX40/CD134, CD3ζ, CD3F, CD3γ, CD3δ, TCRα, TCRζ3, TCRζ, CD32, CD64, CD64, CD45, CD5, CD9, CD22, CD37, CD80, CD86, CD40, CD40L/CD154, VEGFR2, FAS, and FGFR2B, or functional variant thereof. The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD 134, CD 137, CD 154. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s). In some aspects, the transmembrane domain contains a transmembrane portion of CD28.

In some embodiments, the extracellular domain and transmembrane domain can be linked directly or indirectly. In some embodiments, the extracellular domain and transmembrane are linked by a spacer, such as any described herein. In some embodiments, the receptor contains extracellular portion of the molecule from which the transmembrane domain is derived, such as a CD28 extracellular portion.

Among the intracellular signaling domains are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the CAR.

T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the CAR includes one or both of such signaling components.

The receptor, e.g., the CAR, generally includes at least one intracellular signaling component or components. In some aspects, the CAR includes a primary cytoplasmic signaling sequence that regulates primary activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from CD3 zeta chain, FcR gamma, CD3 gamma, CD3 delta and CD3 epsilon. In some embodiments, cytoplasmic signaling molecule(s) in the CAR contain(s) a cytoplasmic signaling domain, portion thereof, or sequence derived from CD3 zeta.

In some embodiments, the receptor includes an intracellular component of a TCR complex, such as a TCR CD3 chain that mediates T-cell activation and cytotoxicity, e.g., CD3 zeta chain. Thus, in some aspects, the antigen-binding portion is linked to one or more cell signaling modules. In some embodiments, cell signaling modules include CD3 transmembrane domain, CD3 intracellular signaling domains, and/or other CD transmembrane domains. In some embodiments, the intracellular component is or includes a CD3-zeta intracellular signaling domain. In some embodiments, the intracellular component is or includes a signaling domain from Fc receptor gamma chain. In some embodiments, the receptor, e.g., CAR, includes the intracellular signaling domain and further includes a portion, such as a transmembrane domain and/or hinge portion, of one or more additional molecules such as CD8, CD4, CD25, or CD 16. For example, in some aspects, the CAR or other chimeric receptor is a chimeric molecule of CD3-zeta (CD3-z) or Fc receptor and a portion of one of CD8, CD4, CD25 or CD16.

In some embodiments, upon ligation of the CAR or other chimeric receptor, the cytoplasmic domain or intracellular signaling domain of the receptor activates at least one of the normal effector functions or responses of the immune cell, e.g., T cell engineered to express the CAR. For example, in some contexts, the CAR induces a function of a T cell such as cytolytic activity or T-helper activity, such as secretion of cytokines or other factors. In some embodiments, a truncated portion of an intracellular signaling domain of an antigen receptor component or costimulatory molecule is used in place of an intact immunostimulatory chain, for example, if it transduces the effector function signal. In some embodiments, the intracellular signaling domain or domains include the cytoplasmic sequences of the T cell receptor (TCR), and in some aspects also those of co-receptors that in the natural context act in concert with such receptors to initiate signal transduction following antigen receptor engagement.

In the context of a natural TCR, full activation generally requires not only signaling through the TCR, but also a costimulatory signal. Thus, in some embodiments, to promote full activation, a component for generating secondary or co-stimulatory signal is also included in the CAR. In other embodiments, the CAR does not include a component for generating a costimulatory signal. In some aspects, an additional CAR is expressed in the same cell and provides the component for generating the secondary or costimulatory signal.

In some embodiments, the chimeric antigen receptor contains an intracellular domain of a T cell costimulatory molecule. In some embodiments, the CAR includes a signaling domain and/or transmembrane portion of a costimulatory receptor, such as CD28, 4-1BB, OX40, DAP10, and ICOS. In some aspects, the same CAR includes both the activating and costimulatory components. In some embodiments, the chimeric antigen receptor contains an intracellular domain derived from a T cell costimulatory molecule or a functional variant thereof, such as between the transmembrane domain and intracellular signaling domain. In some aspects, the T cell costimulatory molecule is CD28 or 41BB. In some aspects, the T cell costimulatory molecule is 41BB.

In some embodiments, the activating domain is included within one CAR, whereas the costimulatory component is provided by another CAR recognizing another antigen. In some embodiments, the CARs include activating or stimulatory CARs, costimulatory CARs, both expressed on the same cell (see WO2014/055668). In some aspects, the cells include one or more stimulatory or activating CAR and/or a costimulatory CAR. In some embodiments, the cells further include inhibitory CARs (iCARs, see Fedorov et al., Sci. Transl. Medicine, 5(215) (December, 2013), such as a CAR recognizing an antigen other than the one associated with and/or specific for the disease or condition whereby an activating signal delivered through the disease-targeting CAR is diminished or inhibited by binding of the inhibitory CAR to its ligand, e.g., to reduce off-target effects.

In certain embodiments, the intracellular signaling domain comprises a CD28 transmembrane and signaling domain linked to a CD3 (e.g., CD3-zeta) intracellular domain. In some embodiments, the intracellular signaling domain comprises a chimeric CD28 and CD137 (4-1BB, TNFRSF9) co-stimulatory domains, linked to a CD3 zeta intracellular domain.

In some embodiments, the CAR encompasses one or more, e.g., two or more, costimulatory domains and an activation domain, e.g., primary activation domain, in the cytoplasmic portion. Exemplary CARs include intracellular components of CD3-zeta, CD28, and 4-1BB.

In some embodiments the intracellular signaling domain includes intracellular components of a 4-1BB signaling domain and a CD3-zeta signaling domain. In some embodiments, the intracellular signaling domain includes intracellular components of a CD28 signaling domain and a CD3zeta signaling domain.

In some embodiments, a CD19 specific CAR includes an anti-CD19 single-chain antibody fragment (scFv), a transmembrane domain such as one derived from human CD80L, a 4-1BB (CD137) co-stimulatory signaling domain, and a CD3ζ signaling domain. In some embodiments, a CD22 specific CAR includes an anti-CD22 scFv, a transmembrane domain such as one derived from human CD8α, a 4-1BB (CD137) co-stimulatory signaling domain, and a CD3C signaling domain. In some embodiments, a CD19/CD22-bispecific CAR includes an anti-CD19 scFv, an anti-CD22 scFv, a transmembrane domain such as one derived from human CD8α, a 4-1BB (CD137) co-stimulatory signaling domain, and a CD3ζ signaling domain.

In some embodiments, the CAR comprises a commercial CAR construct carried by a T cell. Non-limiting examples of commercial CAR-T cell based therapies include brexucabtagene autoleucel (TECARTUS®), axicabtagene ciloleucel (YESCARTA®), idecabtagene vicleucel (ABECMA®), lisocabtagene maraleucel (BREYANZI®), tisagenlecleucel (KYMRIAH®), Descartes-08 and Descartes-li from Cartesian Therapeutics, CTL110 from Novartis, β-BMCA-101 from Poseida Therapeutics, AUTO4 from Autolus Limited, UCARTCS from Cellectis, PBCAR19B and PBCAR269A from Precision Biosciences, FT819 from Fate Therapeutics, and CYAD-211 from Clyad Oncology.

In some embodiments, the antigen binding domain targets an antigen characteristic of an autoimmune or inflammatory disorder. In some embodiments, the ABD binds an antigen associated with an autoimmune or inflammatory disorder. In some instances, the antigen is expressed by a cell associated with an autoimmune or inflammatory disorder. In some embodiments, the autoimmune or inflammatory disorder is selected from chronic graft-vs-host disease (GVHD), lupus, arthritis, immune complex glomerulonephritis, goodpasture syndrome, uveitis, hepatitis, systemic sclerosis or scleroderma, type I diabetes, multiple sclerosis, cold agglutinin disease, Pemphigus vulgaris, Grave's disease, autoimmune hemolytic anemia, Hemophilia A, Primary Sjogren's Syndrome, thrombotic thrombocytopenia purrpura, neuromyelits optica, Evan's syndrome, IgM mediated neuropathy, cryoglobulinemia, dermatomyositis, idiopathic thrombocytopenia, ankylosing spondylitis, bullous pemphigoid, acquired angioedema, chronic urticarial, antiphospholipid demyelinating polyneuropathy, and autoimmune thrombocytopenia or neutropenia or pure red cell aplasias, while exemplary non-limiting examples of alloimmune diseases include allosensitization (see, for example, Blazar et al., 2015, Am. J. Transplant, 15(4):931-41) or xenosensitization from hematopoietic or solid organ transplantation, blood transfusions, pregnancy with fetal allosensitization, neonatal alloimmune thrombocytopenia, hemolytic disease of the newborn, sensitization to foreign antigens such as can occur with replacement of inherited or acquired deficiency disorders treated with enzyme or protein replacement therapy, blood products, and gene therapy. In some embodiments, the antigen characteristic of an autoimmune or inflammatory disorder is selected from a cell surface receptor, an ion channel-linked receptor, an enzyme-linked receptor, a G protein-coupled receptor, receptor tyrosine kinase, tyrosine kinase associated receptor, receptor-like tyrosine phosphatase, receptor serine/threonine kinase, receptor guanylyl cyclase, or histidine kinase associated receptor.

In some embodiments, an antigen binding domain of a CAR binds to a ligand expressed on B cells, plasma cells, or plasmablasts. In some embodiments, an antigen binding domain of a CAR binds to CD10, CD19, CD20, CD22, CD24, CD27, CD38, CD45R, CD138, CD319, BCMA, CD28, TNF, interferon receptors, GM-CSF, ZAP-70, LFA-1, CD3 gamma, CD5 or CD2. See, e.g., US 2003/0077249; WO 2017/058753; WO 2017/058850, the contents of which are herein incorporated by reference.

In some embodiments, the antigen binding domain targets an antigen characteristic of senescent cells, e.g., urokinase-type plasminogen activator receptor (uPAR). In some embodiments, the ABD binds an antigen associated with a senescent cell. In some instances, the antigen is expressed by a senescent cell. In some embodiments, the CAR may be used for treatment or prophylaxis of disorders characterized by the aberrant accumulation of senescent cells, e.g., liver and lung fibrosis, atherosclerosis, diabetes and osteoarthritis.

In some embodiments, the antigen binding domain targets an antigen characteristic of an infectious disease. In some embodiments, the ABD binds an antigen associated with an infectious disease. In some instances, the antigen is expressed by a cell affected by an infectious disease. In some embodiments, wherein the infectious disease is selected from HIV, hepatitis B virus, hepatitis C virus, Human herpes virus, Human herpes virus 8 (HHV-8, Kaposi sarcoma-associated herpes virus (KSHV)), Human T-lymphotrophic virus-1 (HTLV-1), Merkel cell polyomavirus (MCV), Simian virus 40 (SV40), Epstein-Barr virus, CMV, human papillomavirus. In some embodiments, the antigen characteristic of an infectious disease is selected from a cell surface receptor, an ion channel-linked receptor, an enzyme-linked receptor, a G protein-coupled receptor, receptor tyrosine kinase, tyrosine kinase associated receptor, receptor-like tyrosine phosphatase, receptor serine/threonine kinase, receptor guanylyl cyclase, histidine kinase associated receptor, HIV Env, gp120, or CD4-induced epitope on HIV-1 Env.

In some embodiments, an antigen binding domain binds to a cell surface antigen of a cell. In some embodiments, a cell surface antigen is characteristic of (e.g., expressed by) a particular or specific cell type. In some embodiments, a cell surface antigen is characteristic of more than one type of cell.

In some embodiments, a CAR antigen binding domain binds a cell surface antigen characteristic of a T cell, such as a cell surface antigen on a T cell. In some embodiments, an antigen characteristic of a T cell may be a cell surface receptor, a membrane transport protein (e.g., an active or passive transport protein such as, for example, an ion channel protein, a pore-forming protein, etc.), a transmembrane receptor, a membrane enzyme, and/or a cell adhesion protein characteristic of a T cell. In some embodiments, an antigen characteristic of a T cell may be a G protein-coupled receptor, receptor tyrosine kinase, tyrosine kinase associated receptor, receptor-like tyrosine phosphatase, receptor serine/threonine kinase, receptor guanylyl cyclase, or histidine kinase associated receptor.

In some embodiments, the CAR comprises an extracellular antigen binding domain (e.g., antibody or antibody fragment, such as an scFv) that binds to an antigen (e.g. tumor antigen), a spacer (e.g. containing a hinge domain, such as any as described herein), a transmembrane domain (e.g. any as described herein), and an intracellular signaling domain (e.g. any intracellular signaling domain, such as a primary signaling domain or costimulatory signaling domain as described herein). In some embodiments, the intracellular signaling domain is or includes a primary cytoplasmic signaling domain. In some embodiments, the intracellular signaling domain additionally includes an intracellular signaling domain of a costimulatory molecule (e.g., a costimulatory domain). Examples of exemplary components of a CAR are described in Table 2. In provided aspects, the sequences of each component in a CAR can include any combination listed in Table 2.

TABLE 2
CAR components and Exemplary Sequences

| Component | Sequence |
|---|---|
| Extracellular | |
| binding domain | |
| Anti-CD19 | DIQMTQTTSSLSASLGDRVTISCRAS |
| scFv (FMC63) | QDISKYLNWYQQKPDGTVKLLIYHTS |
| SEQ ID NO: 63 | RLHSGVPSRFSGSGSGTDYSLTISNL |
| | EQEDIATYFCQQGNTLPYTFGGGTKL |
| | EITGSTSGSGKPGSGEGSTKGEVKLQ |
| | ESGPGLVAPSQSLSVTCTVSGVSLPD |
| | YGVSWIRQPPRKGLEWLGVIWGSETT |
| | YYNSALKSRLTIIKDNSKSQVFLKMN |
| | SLQTDDTAIYYCAKHYYYGGSYAMDY |
| | WGQGTSVTVSS |
| Anti-CD19 | DIQMTQTTSSLSASLGDRVTISCRAS |
| scFv (FMC63) | QDISKYLNWYQQKPDGTVKLLIYHTS |
| SEQ ID NO: 73 | RLHSGVPSRFSGSGSGTDYSLTISNL |
| | EQEDIATYFCQQGNTLPYTFGGGTKL |
| | EITGGGGSGGGGSGGGGSEVKLQESG |
| | PGLVAPSQSLSVTCTVSGVSLPDYGV |
| | SWIRQPPRKGLEWLGVIWGSETTYYN |
| | SALKSRLTIIKDNSKSQVFLKMNSLQ |
| | TDDTAIYYCAKHYYYGGSYAMDYWGQ |
| | GTSVTVSS |
| Spacer | |
| (e.g. hinge) | |
| IgG4 Hinge | ESKYGPPCPPCP |
| SEQ ID NO: 53 | |
| CD8 Hinge | TTTPAPRPPTPAPTIASQPLSLRPE |
| SEQ ID NO: 142 | |
| CD28 | IEVMYPPPYLDNEKSNGTIIHVKGKH |
| SEQ ID NO: 51 | LCPSPLFPGPSKP |
| Transmembrane | |
| CD8 | ACRPAAGGAVHTRGLDFACDIYIWAP |
| SEQ ID NO: 141 | LAGTCGVLLLSLVITLYC |
| CD28 | FWVLVVVGGVLACYSLLVTVAFIIFW |
| SEQ ID NO: 57 | V |
| CD28 | FWVLVVVGGVLACYSLLVTVAFIIFW |
| SEQ ID NO: 58 | V |
| Costimulatory | |
| domain | |
| CD28 | RSKRSRLLHSDYMNMTPRRPGPTRKH |
| SEQ ID NO: 60 | YQPYAPPRDFAAYRS |
| 4-1BB | KRGRKKLLYIFKQPFMRPVQTTQEED |
| SEQ ID NO: 59 | GCSCRFPEEEEGGCEL |
| Primary | |
| Signaling | |
| Domain | |
| CD3zeta | RVKFSRSADAPAYQQGQNQLYNELNL |
| SEQ ID NO: 61 | GRREEYDVLDKRRGRDPEMGGKPRRK |
| | NPQEGLYNELQKDKMAEAYSEIGMKG |
| | ERRRGKGHDGLYQGLSTATKDTYDAL |
| | HMQALPPR |
| CD3zeta | RVKFSRSADAPAYKQGQNQLYNELNL |
| SEQ ID NO: 62 | GRREEYDVLDKRRGRDPEMGGKPRRK |
| | NPQEGLYNELQKDKMAEAYSEIGMKG |
| | ERRRGKGHDGLYQGLSTATKDTYDAL |
| | HMQALPPR |

In some embodiments, the antigen receptor further includes a marker and/or cells expressing the CAR or other antigen receptor further includes a surrogate marker, such as a cell surface marker, which may be used to confirm transduction or engineering of the cell to express the receptor. In some aspects, the marker includes all or part (e.g., truncated form) of CD34, a NGFR, or epidermal growth factor receptor, such as truncated version of such a cell surface receptor (e.g., tEGFR). In some embodiments, the nucleic acid encoding the marker is operably linked to a polynucleotide encoding for a linker sequence, such as a cleavable linker sequence, e.g., T2A. For example, a marker, and optionally a linker sequence, can be any as disclosed in published patent application No. WO2014031687. For example, the marker can be a truncated EGFR (tEGFR) that is, optionally, linked to a linker sequence, such as a T2A cleavable linker sequence.

In some embodiments, the marker is a molecule, e.g., cell surface protein, not naturally found on T cells or not naturally found on the surface of T cells, or a portion thereof. In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

In some cases, CARs are referred to as first, second, and/or third generation CARs. In some aspects, a first generation CAR is one that solely provides a CD3-chain induced signal upon antigen binding; in some aspects, a second-generation CARs is one that provides such a signal and costimulatory signal, such as one including an intracellular signaling domain from a costimulatory receptor such as CD28 or CD 137; in some aspects, a third generation CAR is one that includes multiple costimulatory domains of different costimulatory receptors.

For example, in some embodiments, the CAR contains an antibody, e.g., an antibody fragment, a transmembrane domain that is or contains a transmembrane portion of CD28 or a functional variant thereof, and an intracellular signaling domain containing a signaling portion of CD28 or functional variant thereof and a signaling portion of CD3 zeta or functional variant thereof. In some embodiments, the CAR contains an antibody, e.g., antibody fragment, a transmembrane domain that is or contains a transmembrane portion of CD28 or a functional variant thereof, and an intracellular signaling domain containing a signaling portion of a 4-IBB or functional variant thereof and a signaling portion of CD3 zeta or functional variant thereof. In some such embodiments, the receptor further includes a spacer containing a portion of an Ig molecule, such as a human Ig molecule, such as an Ig hinge, e.g. an IgG4 hinge, such as a hinge-only spacer.

In some aspects, the spacer contains only a hinge region of an IgG, such as only a hinge of IgG4 or IgG1 In other embodiments, the spacer is or contains an Ig hinge, e.g., an IgG4-derived hinge, optionally linked to a CH2 and/or CH3 domains. In some embodiments, the spacer is an Ig hinge, e.g., an IgG4 hinge, linked to CH2 and CH3 domains. In some embodiments, the spacer is an Ig hinge, e.g., an IgG4 hinge, linked to a CH3 domain only. In some embodiments, the spacer is or comprises a glycine-serine rich sequence or other flexible linker such as known flexible linkers.

For example, in some embodiments, the CAR includes an antibody such as an antibody fragment, including scFvs, a spacer, such as a spacer containing a portion of an immunoglobulin molecule, such as a hinge region and/or one or more constant regions of a heavy chain molecule, such as an Ig-hinge containing spacer, a transmembrane domain containing all or a portion of a CD28-derived transmembrane domain, a CD28-derived intracellular signaling domain, and a CD3 zeta signaling domain. In some embodiments, the CAR includes an antibody or fragment, such as scFv, a spacer such as any of the Ig-hinge containing spacers, a CD28-derived transmembrane domain, a 4-1BB-derived intracellular signaling domain, and a CD3 zeta-derived signaling domain.

The recombinant receptors, such as CARs, expressed by the cells administered to the subject generally recognize or specifically bind to a molecule that is expressed in, associated with, and/or specific for the disease or condition or cells thereof being treated. Upon specific binding to the molecule, e.g., antigen, the receptor generally delivers an immunostimulatory signal, such as an ITAM-transduced signal, into the cell, thereby promoting an immune response targeted to the disease or condition. For example, in some embodiments, the cells express a CAR that specifically binds to an antigen expressed by a cell or tissue of the disease or condition or associated with the disease or condition.

### (ii) T Cell Receptors (TCRs)

In some embodiments, the payload agent is a T cell receptor (TCR) or antigen-binding portion thereof that recognizes a peptide epitope or T cell epitope of a target polypeptide, such as an antigen of a tumor, viral or autoimmune protein.

In some embodiments, a “T cell receptor” or “TCR” is a molecule that contains a variable a and b chains (also known as TCRalpha and TCRbeta, respectively) or a variable g and d chains (also known as TCRalpha and TCRbeta, respectively), or antigen-binding portions thereof, and which is capable of specifically binding to a peptide bound to an MHC molecule. In some embodiments, the TCR is in the ab form. Typically, TCRs that exist in alpha-beta and gamma-delta forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules.

Unless otherwise stated, the term “TCR” should be understood to encompass full TCRs as well as antigen-binding portions or antigen-binding fragments thereof. In some embodiments, the TCR is an intact or full-length TCR, including TCRs in the ab form or gd form. In some embodiments, the TCR is an antigen-binding portion that is less than a full-length TCR but that binds to a specific peptide bound in an MHC molecule, such as binds to an MHC-peptide complex. In some cases, an antigen-binding portion or fragment of a TCR can contain only a portion of the structural domains of a full-length or intact TCR, but yet is able to bind the peptide epitope, such as MHC-peptide complex, to which the full TCR binds. In some cases, an antigen-binding portion contains the variable domains of a TCR, such as variable a chain and variable b chain of a TCR, sufficient to form a binding site for binding to a specific MHC-peptide complex. Generally, the variable chains of a TCR contain complementarity determining regions involved in recognition of the peptide, MHC and/or MHC-peptide complex.

(iii) Chimeric Auto-Antibody Receptor (CAAR)

In some embodiments, the engineered receptor is a chimeric autoantibody receptor (CAAR). In some embodiments, the CAAR binds, e.g., specifically binds, or recognizes, an autoantibody. In some embodiments, a cell expressing the CAAR, such as a T cell engineered to express a CAAR, can be used to bind to and kill autoantibody-expressing cells, but not normal antibody expressing cells. In some embodiments, CAAR-expressing cells can be used to treat an autoimmune disease associated with expression of self-antigens, such as autoimmune diseases. In some embodiments, CAAR-expressing cells can target B cells that ultimately produce the autoantibodies and display the autoantibodies on their cell surfaces, mark these B cells as disease-specific targets for therapeutic intervention. In some embodiments, CAAR-expressing cells can be used to efficiently targeting and killing the pathogenic B cells in autoimmune diseases by targeting the disease-causing B cells using an antigen-specific chimeric autoantibody receptor. In some embodiments, the recombinant receptor is a CAAR, such as any described in U.S. Patent Application Pub. No. US 2017/0051035.

In some embodiments, the CAAR comprises an autoantibody binding domain, a transmembrane domain, and one or more intracellular signaling region or domain (also interchangeably called a cytoplasmic signaling domain or region). In some embodiments, the intracellular signaling region comprises an intracellular signaling domain. In some embodiments, the intracellular signaling domain is or comprises a primary signaling domain, a signaling domain that is capable of stimulating and/or inducing a primary activation signal in a T cell, a signaling domain of a T cell receptor (TCR) component (e.g. an intracellular signaling domain or region of a CD3-zeta) chain or a functional variant or signaling portion thereof), and/or a signaling domain comprising an immunoreceptor tyrosine-based activation motif (ITAM).

In some embodiments, the autoantibody binding domain comprises an autoantigen or a fragment thereof. The choice of autoantigen can depend upon the type of autoantibody being targeted. For example, the autoantigen may be chosen because it recognizes an autoantibody on a target cell, such as a B cell, associated with a particular disease state, e.g. an autoimmune disease, such as an autoantibody-mediated autoimmune disease. In some embodiments, the autoimmune disease includes pemphigus vulgaris (PV). Exemplary autoantigens include desmoglein 1 (Dsg1) and Dsg3.

### (iv) Additional Descriptions of CARs

In certain embodiments, the payload agent is a CAR. CARs (also known as chimeric immunoreceptors, chimeric T cell receptors, or artificial T cell receptors) are receptor proteins that have been engineered to give host cells (e.g., T cells) the new ability to target a specific protein. The receptors are chimeric because they combine both antigen-binding and T cell activating functions into a single receptor. The provided viral vectors may be used to express one or more CARs in a host cell (e.g., a T cell) for use in cell-based therapies against various target antigens. In these embodiments, the CAR may comprise an extracellular binding domain (also referred to as a “binder”) that specifically binds a target antigen, a transmembrane domain, and an intracellular signaling domain. In certain embodiments, the CAR may further comprise one or more additional elements, including one or more signal peptides, one or more extracellular hinge domains, and/or one or more intracellular costimulatory domains. Domains may be directly adjacent to one another, or there may be one or more amino acids linking the domains. The nucleotide sequence encoding a CAR may be derived from a mammalian sequence, for example, a mouse sequence, a primate sequence, a human sequence, or combinations thereof. In the cases where the nucleotide sequence encoding a CAR is non-human, the sequence of the CAR may be humanized. The nucleotide sequence encoding a CAR may also be codon-optimized for expression in a mammalian cell, for example, a human cell. In any of these embodiments, the nucleotide sequence encoding a CAR may be at least 80% identical (e.g., at least 80%, at least 85%, at least 90%, at least 95%, at least 96%, at least 97%, at least 98%, at least 99%, or 100% identical) to any of the nucleotide sequences disclosed herein. The sequence variations may be due to codon-optimalization, humanization, restriction enzyme-based cloning scars, and/or additional amino acid residues linking the functional domains, etc.

In certain embodiments, the CAR may comprise a signal peptide at the N-terminus. Non-limiting examples of signal peptides include CD8α signal peptide, IgK signal peptide, and granulocyte-macrophage colony-stimulating factor receptor subunit alpha (GMCSFR-α, also known as colony stimulating factor 2 receptor subunit alpha (CSF2RA)) signal peptide, and variants thereof, the amino acid sequences of which are provided in Table 3 below.

TABLE 3
Exemplary sequences of signal peptides

| SEQ | | |
|---|---|---|
| ID NO: | Sequence | Description |
| 47 | MALPVTALLLPLALLLHAARP | CD8α signal peptide |
| 48 | METDTLLLWVLLLWVPGSTG | IgK signal peptide |
| 49 | MLLLVTSLLLCELPHPAFLLIP | GMCSFR-α (CSF2RA) |
| | | signal peptide |

In certain embodiments, the extracellular binding domain of the CAR may comprise one or more antibodies specific to one target antigen or multiple target antigens. The antibody may be an antibody fragment, for example, an scFv, or a single-domain antibody fragment, for example, a VHH. In certain embodiments, the scFv may comprise a heavy chain variable region (VH) and a light chain variable region (V_{L}) of an antibody connected by a linker. The VH and the V_{L} may be connected in either order, i.e., V_{H}-linker-V_{L} or V_{L}-linker-VH. Non-limiting examples of linkers include Whitlow linker, (G₄S)ₙ (n can be a positive integer, e.g., 1, 2, 3, 4, 5, 6, etc.) linker, and variants thereof. In certain embodiments, the antigen may be an antigen that is exclusively or preferentially expressed on tumor cells, or an antigen that is characteristic of an autoimmune or inflammatory disease. Exemplary target antigens include, but are not limited to, CD5, CD19, CD20, CD22, CD23, CD30, CD70, Kappa, Lambda, and B cell maturation agent (BCMA), G-protein coupled receptor family C group 5 member D (GPRC5D) (associated with leukemias); CS1/SLAMF7, CD38, CD138, GPRC5D, TACI, and BCMA (associated with myelomas); GD2, HER2, EGFR, EGFRvIII, B7H3, PSMA, PSCA, CAIX, CD171, CEA, CSPG4, EPHA2, FAP, FRα, IL-13Ra, Mesothelin, MUC1, MUC16, and ROR1 (associated with solid tumors). In any of these embodiments, the extracellular binding domain of the CAR can be codon-optimized for expression in a host cell or have variant sequences to increase functions of the extracellular binding domain.

In certain embodiments, the CAR may comprise a hinge domain, also referred to as a spacer. The terms “hinge” and “spacer” may be used interchangeably in the present disclosure. Non-limiting examples of hinge domains include CD8α hinge domain, CD28 hinge domain, IgG4 hinge domain, IgG4 hinge-CH2-CH3 domain, and variants thereof, the amino acid sequences of which are provided in Table 4 below.

TABLE 4
Exemplary sequences of hinge domains

| SEQ ID NO: | Sequence | Description |
|---|---|---|
| 50 | TTTPAPRPPTPAPTIASQPLSLRPEACRPAAGGAVHTRGLDFACD | CD8α hinge domain |
| 51 | IEVMYPPPYLDNEKSNGTIIHVKGKHLCPSPLFPGPSKP | CD28 hinge domain |
| 52 | AAAIEVMYPPPYLDNEKSNGTIIHVKGKHLCPSPLFPGPSKP | CD28 hinge domain |
| 53 | ESKYGPPCPPCP | IgG4 hinge domain |
| 54 | ESKYGPPCPSCP | IgG4 hinge domain |
| 55 | ESKYGPPCPPCPAPEFLGGPSVFLFPPKPKDTLMISRTPEVTCV | IgG4 hinge-CH2—CH3 |
| | VVDVSQEDPEVQFNWYVDGVEVHNAKTKPREEQFNSTYRVV | domain |
| | SVLTVLHQDWLNGKEYKCKVSNKGLPSSIEKTISKAKGQPREP | |
| | QVYTLPPSQEEMTKNQVSLTCLVKGFYPSDIAVEWESNGQPE | |
| | NNYKTTPPVLDSDGSFFLYSRLTVDKSRWQEGNVFSCSVMHE | |
| | ALHNHYTQKSLSLSLGK | |

In certain embodiments, the transmembrane domain of the CAR may comprise a transmembrane region of the alpha, beta, or zeta chain of a T cell receptor, CD28, CD3F, CD45, CD4, CD5, CD8, CD9, CD16, CD22, CD33, CD37, CD64, CD80, CD86, CD134, CD137, CD154, or a functional variant thereof, including the human versions of each of these sequences. In other embodiments, the transmembrane domain may comprise a transmembrane region of CD8α, CD8β, 4-1BB/CD137, CD28, CD34, CD4, FcεRIγ, CD16, OX40/CD134, CD3C, CD3F, CD37, CD3δ, TCRα, TCRβ, TCRζ, CD32, CD64, CD64, CD45, CD5, CD9, CD22, CD37, CD80, CD86, CD40, CD40L/CD154, VEGFR2, FAS, and FGFR2B, or a functional variant thereof, including the human versions of each of these sequences. Table 5 provides the amino acid sequences of a few exemplary transmembrane domains.

TABLE 5
Exemplary sequences of transmembrane domains

| SEQ | | |
|---|---|---|
| ID NO: | Sequence | Description |
| 56 | IYIWAPLAGTCGVL | CD8α transmembrane domain |
| | LLSLVITLYC | |
| 57 | FWVLVVVGGVLAFW | CD28 transmembrane domain |
| | SLLVTVAFIIFWV | |
| 58 | MFWVLVVVGGVLAC | CD28 transmembrane domain |
| | YSLLVTVAFIIFWV | |

In certain embodiments, the intracellular signaling domain and/or intracellular costimulatory domain of the CAR may comprise one or more signaling domains selected from B7-1/CD80, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, PDCD6, 4-1BB/TNFSF9/CD137, 4-1BB Ligand/TNFSF9, BAFF/BLyS/TNFSF13B, BAFF R/TNFRSF13C, CD27/TNFRSF7, CD27 Ligand/TNFSF7, CD30/TNFRSF8, CD30 Ligand/TNFSF8, CD40/TNFRSF5, CD40/TNFSF5, CD40 Ligand/TNFSF5, DR3/TNFRSF25, GITR/TNFRSF18, GITR Ligand/TNFSF18, HVEM/TNFRSF14, LIGHT/TNFSF14, Lymphotoxin-alpha/TNFβ, OX40/TNFRSF4, OX40 Ligand/TNFSF4, RELT/TNFRSF19L, TACI/TNFRSF13B, TL1A/TNFSF15, TNFα, TNF RII/TNFRSF1B, 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, SLAM/CD150, CD2, CD7, CD53, CD82/Kai-1, CD90/Thy1, CD96, CD160, CD200, CD300a/LMIR1, HLA Class I, HLA-DR, Ikaros, Integrin alpha 4/CD49d, integrin alpha 4 beta 1, integrin alpha 4 beta 7/LPAM-1, LAG-3, TCL1A, TCL1B, CRTAM, DAP12, Dectin-1/CLEC7A, DPPIV/CD26, EphB6, TIM-1/KIM-1/HAVCR, TIM-4, TSLP, TSLP R, lymphocyte function associated antigen-1 (LFA-1), NKG2C, CD3ζ, an immunoreceptor tyrosine-based activation motif (ITAM), CD27, CD28, 4-1BB, CD134/OX40, CD30, CD40, PD-1, ICOS, lymphocyte function-associated antigen-1 (LFA-1), CD2, CD7, LIGHT, NKG2C, B7-H3, a ligand that specifically binds with CD83, and a functional variant thereof including the human versions of each of these sequences. In some embodiments, the intracellular signaling domain and/or intracellular costimulatory domain comprises one or more signaling domains selected from a CD3C domain, an ITAM, a CD28 domain, 4-1BB domain, or a functional variant thereof. Table 6 provides the amino acid sequences of a few exemplary intracellular costimulatory and/or signaling domains. In certain embodiments, as in the case of tisagenlecleucel as described below, the CD3ζ signaling domain of SEQ ID NO:99 may have a mutation, e.g., a glutamine (Q) to lysine (K) mutation, at amino acid position 14 (see SEQ ID NO:62).

TABLE 6
Exemplary sequences of intracellular costimulatory and/or signaling domains

| SEQ ID NO: | Sequence | Description |
|---|---|---|
| 59 | KRGRKKLLYIFKQPFMRPVQTTQEEDGCSCRFPEEEEG | 4-1BB costimulatory domain |
| | GCEL | |
| 60 | RSKRSRLLHSDYMNMTPRRPGPTRKHYQPYAPPRDFA | CD28 costimulatory domain |
| | AYRS | |
| 61 | RVKFSRSADAPAYQQGQNQLYNELNLGRREEYDVLDK | CD3ζ signaling domain |
| | RRGRDPEMGGKPRRKNPQEGLYNELQKDKMAEAYSEI | |
| | GMKGERRRGKGHDGLYQGLSTATKDTYDALHMQALP | |
| | PR | |
| 62 | RVKFSRSADAPAYKQGQNQLYNELNLGRREEYDVLDK | CD3ζ signaling domain (with Q |
| | RRGRDPEMGGKPRRKNPQEGLYNELQKDKMAEAYSEI | to K mutation at position 14) |
| | GMKGERRRGKGHDGLYQGLSTATKDTYDALHMQALP | |
| | PR | |

### (a) CD19 CARs

In some embodiments, the CAR is a CD19 CAR (“CD19-CAR”). In some embodiments, the CD19 CAR may comprise a signal peptide, an extracellular binding domain that specifically binds CD19, a hinge domain, a transmembrane domain, an intracellular costimulatory domain, and/or an intracellular signaling domain in tandem.

In some embodiments, the signal peptide of the CD19 CAR comprises a CD8α signal peptide. In some embodiments, the CD8α signal peptide comprises or consists of an amino acid sequence set forth in SEQ ID NO:47 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:47. In some embodiments, the signal peptide comprises an IgK signal peptide. In some embodiments, the IgK signal peptide comprises or consists of an amino acid sequence set forth in SEQ ID NO:48 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:48. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:49 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:49.

In some embodiments, the extracellular binding domain of the CD19 CAR is specific to CD19, for example, human CD19. The extracellular binding domain of the CD19 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the CD19 CAR comprises an scFv derived from the FMC63 monoclonal antibody (FMC63), which comprises the heavy chain variable region (VH) and the light chain variable region (V_{L}) of FMC63 connected by a linker. FMC63 and the derived scFv have been described in Nicholson et al., Mol. Immun. 34(16-17):1157-1165 (1997) and PCT Application Publication No. WO2018/213337, the entire contents of each of which are incorporated by reference herein. In some embodiments, the amino acid sequences of the entire FMC63-derived scFv (also referred to as FMC63 scFv) and its different portions are provided in Table 7 below. In some embodiments, the CD19-specific scFv comprises or consists of an amino acid sequence set forth in SEQ ID NO:63, 64, or 69, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:163, 64, or 69. In some embodiments, the CD19-specific scFv may comprise one or more CDRs having amino acid sequences set forth in SEQ ID NOs: 65-67 and 70-72. In some embodiments, the CD19-specific scFv may comprise a light chain with one or more CDRs having amino acid sequences set forth in SEQ ID NOs: 65-67. In some embodiments, the CD19-specific scFv may comprise a heavy chain with one or more CDRs having amino acid sequences set forth in SEQ ID NOs: 70-72. In any of these embodiments, the CD19-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD19 CAR comprises or consists of the one or more CDRs as described herein.

In some embodiments, the linker linking the V_{H} and the V_{L} portions of the scFv is a Whitlow linker having an amino acid sequence set forth in SEQ ID NO:68. In some embodiments, the Whitlow linker may be replaced by a different linker, for example, a 3×G₄S linker having an amino acid sequence set forth in SEQ ID NO:143, which gives rise to a different FMC63-derived scFv having an amino acid sequence set forth in SEQ ID NO:73. In certain of these embodiments, the CD19-specific scFv comprises or consists of an amino acid sequence set forth in SEQ ID NO:73 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:73.

TABLE 7
Exemplary sequences of anti-CD19 scFv and components

| SEQ ID NO: | Amino Acid Sequence | Description |
|---|---|---|
| 63 | DIQMTQTTSSLSASLGDRVTISCRASQDISKYLN | Anti-CD19 FMC63 scFv |
| | WYQQKPDGTVKLLIYHTSRLHSGVPSRFSGSGSG | entire sequence, with |
| | TDYSLTISNLEQEDIATYFCQQGNTLPYTFGGGTK | Whitlow linker |
| | LEITGSTSGSGKPGSGEGSTKGEVKLQESGPGLV | |
| | APSQSLSVTCTVSGVSLPDYGVSWIRQPPRKGLE | |
| | WLGVIWGSETTYYNSALKSRLTIIKDNSKSQVFLK | |
| | MNSLQTDDTAIYYCAKHYYYGGSYAMDYWGQ | |
| | GTSVTVSS | |
| 64 | DIQMTQTTSSLSASLGDRVTISCRASQDISKYLN | Anti-CD19 FMC63 scFv light |
| | WYQQKPDGTVKLLIYHTSRLHSGVPSRFSGSGSG | chain variable region |
| | TDYSLTISNLEQEDIATYFCQQGNTLPYTFGGGTK | |
| | LEIT | |
| 65 | QDISKY | Anti-CD19 FMC63 scFv light |
| | | chain CDR1 |
| 66 | HTS | Anti-CD19 FMC63 scFv light |
| | | chain CDR2 |
| 67 | QQGNTLPYT | Anti-CD19 FMC63 scFv light |
| | | chain CDR3 |
| 68 | GSTSGSGKPGSGEGSTKG | Whitlow linker |
| 69 | EVKLQESGPGLVAPSQSLSVTCTVSGVSLPDYGV | Anti-CD19 FMC63 scFv |
| | SWIRQPPRKGLEWLGVIWGSETTYYNSALKSRLT | heavy chain variable region |
| | IIKDNSKSQVFLKMNSLQTDDTAIYYCAKHYYYG | |
| | GSYAMDYWGQGTSVTVSS | |
| 70 | GVSLPDYG | Anti-CD19 FMC63 scFv |
| | | heavy chain CDR1 |
| 71 | IWGSETT | Anti-CD19 FMC63 scFv |
| | | heavy chain CDR2 |
| 72 | AKHYYYGGSYAMDY | Anti-CD19 FMC63 scFv |
| | | heavy chain CDR3 |
| 73 | DIQMTQTTSSLSASLGDRVTISCRASQDISKYLN | Anti-CD19 FMC63 scFv |
| | WYQQKPDGTVKLLIYHTSRLHSGVPSRFSGSGSG | entire sequence, with 3xG₄S |
| | TDYSLTISNLEQEDIATYFCQQGNTLPYTFGGGTK | linker |
| | LEITGGGGSGGGGSGGGGSEVKLQESGPGLVAP | |
| | SQSLSVTCTVSGVSLPDYGVSWIRQPPRKGLEW | |
| | LGVIWGSETTYYNSALKSRLTIIKDNSKSQVFLKM | |
| | NSLQTDDTAIYYCAKHYYYGGSYAMDYWGQGT | |
| | SVTVSS | |
| 143 | GGGGSGGGGSGGGGS | 3xG₄S linker |

In some embodiments, the extracellular binding domain of the CD19 CAR is derived from an antibody specific to CD19, including, for example, SJ25C1 (Bejceck et al., Cancer Res. 55:2346-2351 (1995)), HD37 (Pezutto et al., J. Immunol. 138(9):2793-2799 (1987)), 4G7 (Meeker et al., Hybridoma 3:305-320 (1984)), B43 (Bejcek (1995)), BLY3 (Bejcek (1995)), B4 (Freedman et al., 70:418-427 (1987)), B4 HB 12b (Kansas & Tedder, J. Immunol. 147:4094-4102 (1991); Yazawa et al., Proc. Natl. Acad. Sci. USA 102:15178-15183 (2005), Herbst et al., J. Pharmacol. Exp. Ther. 335:213-222 (2010)), BU12 (Callard et al., J. Immunology, 148(10): 2983-2987 (1992)), and CLB-CD19 (De Rie Cell. Immunol. 118:368-381(1989)). In any of these embodiments, the extracellular binding domain of the CD 19 CAR can comprise or consist of the V_{H}, the V_{L}, and/or one or more CDRs of any of the antibodies.

In some embodiments, the hinge domain of the CD19 CAR comprises a CD8α hinge domain, for example, a human CD8α hinge domain. In some embodiments, the CD8α hinge domain comprises or consists of an amino acid sequence set forth in SEQ ID NO:50 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:50. In some embodiments, the hinge domain comprises a CD28 hinge domain, for example, a human CD28 hinge domain. In some embodiments, the CD28 hinge domain comprises or consists of an amino acid sequence set forth in SEQ ID NO:51 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:51. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:53 or SEQ ID NO:54, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:53 or SEQ ID NO:54. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:55 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:55.

In some embodiments, the transmembrane domain of the CD19 CAR comprises a CD8α transmembrane domain, for example, a human CD8α transmembrane domain. In some embodiments, the CD8α transmembrane domain comprises or consists of an amino acid sequence set forth in SEQ ID NO:56 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:56. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:57 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:57.

In some embodiments, the intracellular costimulatory domain of the CD19 CAR comprises a 4-1BB costimulatory domain. 4-1BB, also known as CD137, transmits a potent costimulatory signal to T cells, promoting differentiation and enhancing long-term survival of T lymphocytes. In some embodiments, the 4-1BB costimulatory domain is human. In some embodiments, the 4-1BB costimulatory domain comprises or consists of an amino acid sequence set forth in SEQ ID NO:59 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:59. In some embodiments, the intracellular costimulatory domain comprises a CD28 costimulatory domain. CD28 is another co-stimulatory molecule on T cells. In some embodiments, the CD28 costimulatory domain is human. In some embodiments, the CD28 costimulatory domain comprises or consists of an amino acid sequence set forth in SEQ ID NO:60 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:60. In some embodiments, the intracellular costimulatory domain of the CD19 CAR comprises a 4-1BB costimulatory domain and a CD28 costimulatory domain as described.

In some embodiments, the intracellular signaling domain of the CD19 CAR comprises a CD3 zeta (ζ) signaling domain. CD3 zeta associates with T cell receptors (TCRs) to produce a signal and contains immunoreceptor tyrosine-based activation motifs (ITAMs). The CD3 zeta signaling domain refers to amino acid residues from the cytoplasmic domain of the zeta chain that are sufficient to functionally transmit an initial signal necessary for T cell activation. In some embodiments, the CD3 zeta signaling domain is human. In some embodiments, the CD3 zeta signaling domain comprises or consists of an amino acid sequence set forth in SEQ ID NO:61 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:61.

In some embodiments, the payload agent is a CD119 CAR, including, for example, a CD19 CAR comprising the CD19-specific scFv having sequences set forth in SEQ ID NO:63 or SEQ ID NO:73, the CD8α hinge domain of SEQ ID NO:50, the CD8α transmembrane domain of SEQ ID NO:56, the 4-1BB costimulatory domain of SEQ ID NO:59, the CD3ζ signaling domain of SEQ ID NO:61, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the CD19 CAR may additionally comprise a signal peptide (e.g., a CD8α signal peptide) as described.

In some embodiments, the payload agent is a CD119 CAR, including, for example, a CD19 CAR comprising the CD19-specific scFv having sequences set forth in SEQ ID NO:63 or SEQ ID NO:73, the IgG4 hinge domain of SEQ ID NO:53 or SEQ ID NO:54, the CD28 transmembrane domain of SEQ ID NO:57, the 4-1BB costimulatory domain of SEQ ID NO:59, the CD3ζ signaling domain of SEQ ID NO:61, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the CD19 CAR may additionally comprise a signal peptide (e.g., a CD8α signal peptide) as described.

In some embodiments, the payload agent is a CD119 CAR, including, for example, a CD19 CAR comprising the CD19-specific scFv having sequences set forth in SEQ ID NO:63 or SEQ ID NO:73, the CD28 hinge domain of SEQ ID NO:51, the CD28 transmembrane domain of SEQ ID NO:57, the CD28 costimulatory domain of SEQ ID NO:60, the CD3ζ signaling domain of SEQ ID NO:61, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the CD19 CAR may additionally comprise a signal peptide (e.g., a CD8α signal peptide) as described.

In some embodiments, the payload agent is a CD19 CAR as encoded by the sequence set forth in SEQ ID NO:74 or a sequence at least 80% identical (e.g., at least 80%, at least 85%, at least 90%, at least 95%, at least 96%, at least 97%, at least 98%, at least 99%, or 100% identical) to the nucleotide sequence set forth in SEQ ID NO:74 (see Table 8). The encoded CD19 CAR has a corresponding amino acid sequence set forth in SEQ ID NO:75 or is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:75, with the following components: CD8α signal peptide, FMC63 scFv (V_{L}—Whitlow linker-V_{H}), CD8α hinge domain, CD8α transmembrane domain, 4-1BB costimulatory domain, and CD3ζ signaling domain.

In some embodiments, the payload agent is a commercially available embodiment of a CD19 CAR. Non-limiting examples of commercially available embodiments of CD19 CARs include tisagenlecleucel, lisocabtagene maraleucel, axicabtagene ciloleucel, and brexucabtagene autoleucel.

In some embodiments, the CAR is tisagenlecleucel or portions thereof. Tisagenlecleucel comprises a CD19 CAR with the following components: CD8α signal peptide, FMC63 scFv (V_{L}-3×G₄S linker-V_{H}), CD8α hinge domain, CD8α transmembrane domain, 4-1BB costimulatory domain, and CD3ζ signaling domain. The nucleotide and amino acid sequence of the CD19 CAR in tisagenlecleucel are provided in Table 8, with annotations of the sequences provided in Table 9.

In some embodiments, the CAR is lisocabtagene maraleucel or portions thereof. Lisocabtagene maraleucel comprises a CD19 CAR with the following components: GMCSFR-α or CSF2RA signal peptide, FMC63 scFv (V_{L}—Whitlow linker-VH), IgG4 hinge domain, CD28 transmembrane domain, 4-1BB costimulatory domain, and CD3ζ signaling domain. The nucleotide and amino acid sequence of the CD19 CAR in lisocabtagene maraleucel are provided in Table 8, with annotations of the sequences provided in Table 10.

In some embodiments, the CAR is axicabtagene ciloleucel or portions thereof. Axicabtagene ciloleucel comprises a CD19 CAR with the following components: GMCSFR-α or CSF2RA signal peptide, FMC63 scFv (V_{L}-Whitlow linker-VH), CD28 hinge domain, CD28 transmembrane domain, CD28 costimulatory domain, and CD3ζ signaling domain. The nucleotide and amino acid sequence of the CD19 CAR in axicabtagene ciloleucel are provided in Table 8, with annotations of the sequences provided in Table 11.

In some embodiments, the CAR is brexucabtagene autoleucel or portions thereof. Brexucabtagene autoleucel comprises a CD19 CAR with the following components: GMCSFR-α signal peptide, FMC63 scFv, CD28 hinge domain, CD28 transmembrane domain, CD28 costimulatory domain, and CD3 signaling domain.

In some embodiments, the CAR is encoded by the sequence set forth in SEQ ID NO: 76, 78, or 80, or a sequence at least 80% identical (e.g., at least 80%, at least 85%, at least 90%, at least 95%, at least 96%, at least 97%, at least 98%, at least 99%, or 100% identical) to the nucleotide sequence set forth in SEQ ID NO: 76, 78, or 80. The encoded CD19 CAR has a corresponding amino acid sequence set forth in SEQ ID NO: 77, 79, or 81, respectively, or is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 77, 79, or 81, respectively.

TABLE 8
Exemplary sequences of CD19 CARs

| SEQ ID NO: | Sequence | Description |
|---|---|---|
| 74 | atggccttaccagtgaccgccttgctcctgccgctggccttgctgctccac | Exemplary CD19 |
| | gccgccaggccggacatccagatgacacagactacatcctccctgtctg | CAR nucleotide |
| | cctctctgggagacagagtcaccatcagttgcagggcaagtcaggacat | sequence |
| | tagtaaatatttaaattggtatcagcagaaaccagatggaactgttaaa | |
| | ctcctgattaccatacatcaagattacactcaggagtcccatcaaggtt | |
| | cagtggcagtgggtctggaacagattattctctcaccattagcaacctgg | |
| | agcaagaagatattgccacttacttttgccaacagggtaatacgcttccg | |
| | tacacgttcggaggggggaccaagctggagatcacaggctccacctctg | |
| | gatccggcaagcccggatctggcgagggatccaccaagggcgaggtga | |
| | aactgcaggagtcaggacctggcctggtggcgccctcacagagcctgtc | |
| | cgtcacatgcactgtctcaggggtctcattacccgactatggtgtaagct | |
| | ggattcgccagcctccacgaaagggtctggagtggctgggagtaatatg | |
| | gggtagtgaaaccacatactataattcagctctcaaatccagactgacc | |
| | atcatcaaggacaactccaagagccaagttttcttaaaaatgaacagtc | |
| | tgcaaactgatgacacagccatttactactgtgccaaacattattactac | |
| | ggtggtagctatgctatggactactggggccaaggaacctcagtcaccg | |
| | tctcctcaaccacgacgccagcgccgcgaccaccaacaccggcgccca | |
| | ccatcgcgtcgcagcccctgtccctgcgcccagaggcgtgccggccagc | |
| | ggcggggggcgcagtgcacacgagggggctggacttcgcctgtgatatc | |
| | tacatctgggcgcccttggccgggacttgtggggtccttctcctgtcactg | |
| | gttatcaccctttactgcaaacggggcagaaagaaactcctgtatatatt | |
| | caaacaaccatttatgagaccagtacaaactactcaagaggaagatgg | |
| | ctgtagctgccgatttccagaagaagaagaaggaggatgtgaactgag | |
| | agtgaagttcagcaggagcgcagacgcccccgcgtaccagcagggcca | |
| | gaaccagctctataacgagctcaatctaggacgaagagaggagtacga | |
| | tgttttggacaagagacgtggccgggaccctgagatggggggaaagcc | |
| | gagaaggaagaaccctcaggaaggcctgtacaatgaactgcagaaag | |
| | ataagatggcggaggcctacagtgagattgggatgaaaggcgagcgcc | |
| | ggaggggcaaggggcacgatggcctttaccagggtctcagtacagcca | |
| | ccaaggacacctacgacgcccttcacatgcaggccctgccccctcgc | |
| 75 | MALPVTALLLPLALLLHAARPDIQMTQTTSSLSASLGDRV | Exemplary CD19 |
| | TISCRASQDISKYLNWYQQKPDGTVKLLIYHTSRLHSGVPS | CAR amino acid |
| | RFSGSGSGTDYSLTISNLEQEDIATYFCQQGNTLPYTFGGG | sequence |
| | TKLEITGSTSGSGKPGSGEGSTKGEVKLQESGPGLVAPSQS | |
| | LSVTCTVSGVSLPDYGVSWIRQPPRKGLEWLGVIWGSET | |
| | TYYNSALKSRLTIIKDNSKSQVFLKMNSLQTDDTAIYYCAK | |
| | HYYYGGSYAMDYWGQGTSVTVSSTTTPAPRPPTPAPTIA | |
| | SQPLSLRPEACRPAAGGAVHTRGLDFACDIYIWAPLAGTC | |
| | GVLLLSLVITLYCKRGRKKLLYIFKQPFMRPVQTTQEEDGC | |
| | SCRFPEEEEGGCELRVKFSRSADAPAYQQGQNQLYNELN | |
| | LGRREEYDVLDKRRGRDPEMGGKPRRKNPQEGLYNELQ | |
| | KDKMAEAYSEIGMKGERRRGKGHDGLYQGLSTATKDTY | |
| | DALHMQALPPR | |
| 76 | atggccttaccagtgaccgccttgctcctgccgctggccttgctgctccac | Tisagenlecleucel |
| | gccgccaggccggacatccagatgacacagactacatcctccctgtctg | CD19 CAR |
| | cctctctgggagacagagtcaccatcagttgcagggcaagtcaggacat | nucleotide sequence |
| | tagtaaatatttaaattggtatcagcagaaaccagatggaactgttaaa | |
| | ctcctgatctaccatacatcaagattacactcaggagtcccatcaaggtt | |
| | cagtggcagtgggtctggaacagattattctctcaccattagcaacctgg | |
| | agcaagaagatattgccacttacttttgccaacagggtaatacgcttccg | |
| | tacacgttcggaggggggaccaagctggagatcacaggtggcggtggc | |
| | tcgggcggtggtgggtcgggtggcggcggatctgaggtgaaactgcag | |
| | gagtcaggacctggcctggtggcgccctcacagagcctgtccgtcacat | |
| | gcactgtctcaggggtctcattacccgactatggtgtaagctggattcgc | |
| | cagcctccacgaaagggtctggagtggctgggagtaatatggggtagtg | |
| | aaaccacatactataattcagctctcaaatccagactgaccatcatcaag | |
| | gacaactccaagagccaagttttcttaaaaatgaacagtctgcaaactg | |
| | atgacacagccatttactactgtgccaaacattattactacggtggtagc | |
| | tatgctatggactactggggccaaggaacctcagtcaccgtctcctcaac | |
| | cacgacgccagcgccgcgaccaccaacaccggcgcccaccatcgcgtc | |
| | gcagcccctgtccctgcgcccagaggcgtgccggccagcggcgggggg | |
| | cgcagtgcacacgagggggctggacttcgcctgtgatatctacatctggg | |
| | cgcccttggccgggacttgtggggtccttctcctgtcactggttatcaccct | |
| | ttactgcaaacggggcagaaagaaactcctgtatatattcaaacaacca | |
| | tttatgagaccagtacaaactactcaagaggaagatggctgtagctgcc | |
| | gatttccagaagaagaagaaggaggatgtgaactgagagtgaagttca | |
| | gcaggagcgcagacgcccccgcgtacaagcagggccagaaccagctct | |
| | ataacgagctcaatctaggacgaagagaggagtacgatgttttggaca | |
| | agagacgtggccgggaccctgagatggggggaaagccgagaaggaag | |
| | aaccctcaggaaggcctgtacaatgaactgcagaaagataagatggcg | |
| | gaggcctacagtgagattgggatgaaaggcgagcgccggaggggcaa | |
| | ggggcacgatggcctttaccagggtctcagtacagccaccaaggacacc | |
| | tacgacgcccttcacatgcaggccctgccccctcgc | |
| 77 | MALPVTALLLPLALLLHAARPDIQMTQTTSSLSASLGDRV | Tisagenlecleucel |
| | TISCRASQDISKYLNWYQQKPDGTVKLLIYHTSRLHSGVPS | CD19 CAR amino |
| | RFSGSGSGTDYSLTISNLEQEDIATYFCQQGNTLPYTFGGG | acid sequence |
| | TKLEITGGGGSGGGGSGGGGSEVKLQESGPGLVAPSQSL | |
| | SVTCTVSGVSLPDYGVSWIRQPPRKGLEWLGVIWGSETT | |
| | YYNSALKSRLTIIKDNSKSQVFLKMNSLQTDDTAIYYCAKH | |
| | YYYGGSYAMDYWGQGTSVTVSSTTTPAPRPPTPAPTIAS | |
| | QPLSLRPEACRPAAGGAVHTRGLDFACDIYIWAPLAGTC | |
| | GVLLLSLVITLYCKRGRKKLLYIFKQPFMRPVQTTQEEDGC | |
| | SCRFPEEEEGGCELRVKFSRSADAPAYKQGQNQLYNELNL | |
| | GRREEYDVLDKRRGRDPEMGGKPRRKNPQEGLYNELQK | |
| | DKMAEAYSEIGMKGERRRGKGHDGLYQGLSTATKDTYD | |
| | ALHMQALPPR | |
| 78 | atgctgctgctggtgaccagcctgctgctgtgcgagctgccccaccccgc | Lisocabtagene |
| | ctttctgctgatccccgacatccagatgacccagaccacctccagcctga | maraleucel CD19 |
| | gcgccagcctgggcgaccgggtgaccatcagctgccgggccagccagg | CAR nucleotide |
| | acatcagcaagtacctgaactggtatcagcagaagcccgacggcaccg | sequence |
| | tcaagctgctgatctaccacaccagccggctgcacagcggcgtgcccag | |
| | ccggtttagcggcagcggctccggcaccgactacagcctgaccatctcc | |
| | aacctggaacaggaagatatcgccacctacttttgccagcagggcaaca | |
| | cactgccctacacctttggcggcggaacaaagctggaaatcaccggcag | |
| | cacctccggcagcggcaagcctggcagcggcgagggcagcaccaagg | |
| | gcgaggtgaagctgcaggaaagcggccctggcctggtggcccccagcc | |
| | agagcctgagcgtgacctgcaccgtgagcggcgtgagcctgcccgacta | |
| | cggcgtgagctggatccggcagccccccaggaagggcctggaatggct | |
| | gggcgtgatctggggcagcgagaccacctactacaacagcgccctgaa | |
| | gagccggctgaccatcatcaaggacaacagcaagagccaggtgttcct | |
| | gaagatgaacagcctgcagaccgacgacaccgccatctactactgcgc | |
| | caagcactactactacggcggcagctacgccatggactactggggccag | |
| | ggcaccagcgtgaccgtgagcagcgaatctaagtacggaccgccctgc | |
| | cccccttgccctatgttctgggtgctggtggtggtcggaggcgtgctggcc | |
| | tgctacagcctgctggtcaccgtggccttcatcatcttttgggtgaaacgg | |
| | ggcagaaagaaactcctgtatatattcaaacaaccatttatgagaccag | |
| | tacaaactactcaagaggaagatggctgtagctgccgatttccagaaga | |
| | agaagaaggaggatgtgaactgcgggtgaagttcagcagaagcgccg | |
| | acgcccctgcctaccagcagggccagaatcagctgtacaacgagctga | |
| | acctgggcagaagggaagagtacgacgtcctggataagcggagaggc | |
| | cgggaccctgagatgggcggcaagcctcggcggaagaacccccagga | |
| | aggcctgtataacgaactgcagaaagacaagatggccgaggcctacag | |
| | cgagatcggcatgaagggcgagcggaggcggggcaagggccacgacg | |
| | gcctgtatcagggcctgtccaccgccaccaaggatacctacgacgccct | |
| | gcacatgcaggccctgcccccaagg | |
| 79 | MLLLVTSLLLCELPHPAFLLIPDIQMTQTTSSLSASLGDRVT | Lisocabtagene |
| | ISCRASQDISKYLNWYQQKPDGTVKLLIYHTSRLHSGVPSR | maraleucel CD19 |
| | FSGSGSGTDYSLTISNLEQEDIATYFCQQGNTLPYTFGGGT | CAR amino acid |
| | KLEITGSTSGSGKPGSGEGSTKGEVKLQESGPGLVAPSQSL | sequence |
| | SVTCTVSGVSLPDYGVSWIRQPPRKGLEWLGVIWGSETT | |
| | YYNSALKSRLTIIKDNSKSQVFLKMNSLQTDDTAIYYCAKH | |
| | YYYGGSYAMDYWGQGTSVTVSSESKYGPPCPPCPMFW | |
| | VLVVVGGVLACYSLLVTVAFIIFWVKRGRKKLLYIFKQPFM | |
| | RPVQTTQEEDGCSCRFPEEEEGGCELRVKFSRSADAPAYQ | |
| | QGQNQLYNELNLGRREEYDVLDKRRGRDPEMGGKPRRK | |
| | NPQEGLYNELQKDKMAEAYSEIGMKGERRRGKGHDGLY | |
| | QGLSTATKDTYDALHMQALPPR | |
| 80 | atgcttctcctggtgacaagccttctgctctgtgagttaccacacccagca | Axicabtagene |
| | ttcctcctgatcccagacatccagatgacacagactacatcctccctgtct | ciloleucel CD19 CAR |
| | gcctctctgggagacagagtcaccatcagttgcagggcaagtcaggaca | nucleotide sequence |
| | ttagtaaatatttaaattggtatcagcagaaaccagatggaactgttaaa | |
| | ctcctgatctaccatacatcaagattacactcaggagtcccatcaaggtt | |
| | cagtggcagtgggtctggaacagattattctctcaccattagcaacctgg | |
| | agcaagaagatattgccacttacttttgccaacagggtaatacgcttccg | |
| | tacacgttcggaggggggactaagttggaaataacaggctccacctctg | |
| | gatccggcaagcccggatctggcgagggatccaccaagggcgaggtga | |
| | aactgcaggagtcaggacctggcctggtggcgccctcacagagcctgtc | |
| | cgtcacatgcactgtctcaggggtctcattacccgactatggtgtaagct | |
| | ggattcgccagcctccacgaaagggtctggagtggctgggagtaatatg | |
| | gggtagtgaaaccacatactataattcagctctcaaatccagactgacc | |
| | atcatcaaggacaactccaagagccaagttttcttaaaaatgaacagtc | |
| | tgcaaactgatgacacagccatttactactgtgccaaacattattactac | |
| | ggtggtagctatgctatggactactggggtcaaggaacctcagtcaccgt | |
| | ctcctcagcggccgcaattgaagttatgtatcctcctccttacctagacaa | |
| | tgagaagagcaatggaaccattatccatgtgaaagggaaacacctttgt | |
| | ccaagtcccctatttcccggaccttctaagcccttttgggtgctggtggtg | |
| | gttgggggagtcctggcttgctatagcttgctagtaacagtggcctttatt | |
| | attttctgggtgaggagtaagaggagcaggctcctgcacagtgactaca | |
| | tgaacatgactccccgccgccccgggcccacccgcaagcattaccagcc | |
| | ctatgccccaccacgcgacttcgcagcctatcgctccagagtgaagttca | |
| | gcaggagcgcagacgcccccgcgtaccagcagggccagaaccagctct | |
| | ataacgagctcaatctaggacgaagagaggagtacgatgttttggaca | |
| | agagacgtggccgggaccctgagatggggggaaagccgagaaggaag | |
| | aaccctcaggaaggcctgtacaatgaactgcagaaagataagatggcg | |
| | gaggcctacagtgagattgggatgaaaggcgagcgccggaggggcaa | |
| | ggggcacgatggcctttaccagggtctcagtacagccaccaaggacacc | |
| | tacgacgcccttcacatgcaggccctgccccctcgc | |
| 81 | MLLLVTSLLLCELPHPAFLLIPDIQMTQTTSSLSASLGDRVT | Axicabtagene |
| | ISCRASQDISKYLNWYQQKPDGTVKLLIYHTSRLHSGVPSR | ciloleucel CD19 CAR |
| | FSGSGSGTDYSLTISNLEQEDIATYFCQQGNTLPYTFGGGT | amino acid |
| | KLEITGSTSGSGKPGSGEGSTKGEVKLQESGPGLVAPSQSL | sequence |
| | SVTCTVSGVSLPDYGVSWIRQPPRKGLEWLGVIWGSETT | |
| | YYNSALKSRLTIIKDNSKSQVFLKMNSLQTDDTAIYYCAKH | |
| | YYYGGSYAMDYWGQGTSVTVSSAAAIEVMYPPPYLDNE | |
| | KSNGTIIHVKGKHLCPSPLFPGPSKPFWVLVVVGGVLACY | |
| | SLLVTVAFIIFWVRSKRSRLLHSDYMNMTPRRPGPTRKHY | |
| | QPYAPPRDFAAYRSRVKFSRSADAPAYQQGQNQLYNEL | |
| | NLGRREEYDVLDKRRGRDPEMGGKPRRKNPQEGLYNEL | |
| | QKDKMAEAYSEIGMKGERRRGKGHDGLYQGLSTATKDT | |
| | YDALHMQALPPR | |

TABLE 9
Annotation of tisagenlecleucel CD19 CAR sequences

| | Nucleotide | Amino Acid |
|---|---|---|
| | Sequence | Sequence |
| Feature | Position | Position |
| CD8α signal peptide | 1-63 | 1-21 |
| FMC63 scFv | 64-789 | 22-263 |
| (V_{L}-3xG₄S | | |
| linker-V_{H}) | | |
| CD8α hinge domain | 790-924 | 264-308 |
| CD8α transmembrane domain | 925-996 | 309-332 |
| 4-1BB costimulatory domain | 997-1122 | 333-374 |
| CD3ζ signaling domain | 1123-1458 | 375-486 |

TABLE 10
Annotation of lisocabtagene maraleucel CD19 CAR sequences

| | Nucleotide | Amino Acid |
|---|---|---|
| | Sequence | Sequence |
| Feature | Position | Position |
| GMCSFR-α signal peptide | 1-66 | 1-22 |
| FMC63 scFv | 67-801 | 23-267 |
| (V_{L}-Whitlow linker-V_{H}) | | |
| IgG4 hinge domain | 802-837 | 268-279 |
| CD28 transmembrane domain | 838-921 | 280-307 |
| 4-1BB costimulatory domain | 922-1047 | 308-349 |
| CD3ζ signaling domain | 1048-1383 | 350-461 |

TABLE 11
Annotation of axicabtagene ciloleucel CD19 CAR sequences

| | Nucleotide | Amino Acid |
|---|---|---|
| | Sequence | Sequence |
| Feature | Position | Position |
| CSF2RA signal peptide | 1-66 | 1-22 |
| FMC63 scFv | 67-801 | 23-267 |
| (V_{L}-Whitlow linker-V_{H}) | | |
| CD28 hinge domain | 802-927 | 268-309 |
| CD28 transmembrane domain | 928-1008 | 310-336 |
| CD28 costimulatory domain | 1009-1131 | 337-377 |
| CD3 zeta signaling domain | 1132-1467 | 378-489 |

In some embodiments, the CAR is encoded by the sequence set forth in SEQ ID NO: 76, 78, or 80, or a sequence at least 80% identical (e.g., at least 80%, at least 85%, at least 90%, at least 95%, at least 96%, at least 97%, at least 98%, at least 99%, or 100% identical) to the nucleotide sequence set forth in SEQ ID NO: 76, 78, or 80. The encoded CD19 CAR has a corresponding amino acid sequence set forth in SEQ ID NO: 77, 79, or 81, respectively, is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 77, 79, or 81, respectively.

### (b) CD20 CARs

In some embodiments, the CAR is a CD20 CAR (“CD20-CAR”). CD20 is an antigen found on the surface of B cells as early at the pro-B phase and progressively at increasing levels until B cell maturity, as well as on the cells of most B-cell neoplasms. CD20 positive cells are also sometimes found in cases of Hodgkins disease, myeloma, and thymoma. In some embodiments, the CD20 CAR may comprise a signal peptide, an extracellular binding domain that specifically binds CD20, a hinge domain, a transmembrane domain, an intracellular costimulatory domain, and/or an intracellular signaling domain in tandem.

In some embodiments, the signal peptide of the CD20 CAR comprises a CD8α signal peptide. In some embodiments, the CD8α signal peptide comprises or consists of an amino acid sequence set forth in SEQ ID NO:47 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:47. In some embodiments, the signal peptide comprises an IgK signal peptide. In some embodiments, the IgK signal peptide comprises or consists of an amino acid sequence set forth in SEQ ID NO:48 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:48. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:49 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:49.

In some embodiments, the extracellular binding domain of the CD20 CAR is specific to CD20, for example, human CD20. The extracellular binding domain of the CD20 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the CD20 CAR is derived from an antibody specific to CD20, including, for example, Leu16, IF5, 1.5.3, rituximab, obinutuzumab, ibritumomab, ofatumumab, tositumumab, odronextamab, veltuzumab, ublituximab, and ocrelizumab. In any of these embodiments, the extracellular binding domain of the CD20 CAR can comprise or consist of the V_{H}, the V_{L}, and/or one or more CDRs of any of the antibodies.

In some embodiments, the extracellular binding domain of the CD20 CAR comprises an scFv derived from the Leu16 monoclonal antibody, which comprises the heavy chain variable region (V_{H}) and the light chain variable region (V_{L}) of Leu16 connected by a linker. See Wu et al., Protein Engineering. 14(12):1025-1033 (2001). In some embodiments, the linker is a 3×G₄S linker. In other embodiments, the linker is a Whitlow linker as described herein. In some embodiments, the amino acid sequences of different portions of the entire Leu16-derived scFv (also referred to as Leu16 scFv) and its different portions are provided in Table 12 below. In some embodiments, the CD20-specific scFv comprises or consists of an amino acid sequence set forth in SEQ ID NO:82, 83, or 87, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:82, 83, or 87. In some embodiments, the CD20-specific scFv may comprise one or more CDRs having amino acid sequences set forth in SEQ ID NOs: 84-86, 88, 89, and 144. In some embodiments, the CD20-specific scFv may comprise a light chain with one or more CDRs having amino acid sequences set forth in SEQ ID NOs: 84-86. In some embodiments, the CD20-specific scFv may comprise a heavy chain with one or more CDRs having amino acid sequences set forth in SEQ ID NOs: 88, 89, and 144. In any of these embodiments, the CD20-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD20 CAR comprises or consists of the one or more CDRs as described herein.

TABLE 12
Exemplary sequences of anti-CD20 scFv and components

| SEQ ID NO: | Amino Acid Sequence | Description |
|---|---|---|
| 82 | DIVLTQSPAILSASPGEKVTMTCRASSSVNYMD | Anti-CD20 Leu16 scFv |
| | WYQKKPGSSPKPWIYATSNLASGVPARFSGSGS | entire sequence, with |
| | GTSYSLTISRVEAEDAATYYCQQWSFNPPTFGG | Whitlow linker |
| | GTKLEIKGSTSGSGKPGSGEGSTKGEVQLQQSGA | |
| | ELVKPGASVKMSCKASGYTFTSYNMHWVKQTP | |
| | GQGLEWIGAIYPGNGDTSYNQKFKGKATLTADK | |
| | SSSTAYMQLSSLTSEDSADYYCARSNYYGSSYWF | |
| | FDVWGAGTTVTVSS | |
| 83 | DIVLTQSPAILSASPGEKVTMTCRASSSVNYMD | Anti-CD20 Leu16 scFv light |
| | WYQKKPGSSPKPWIYATSNLASGVPARFSGSGS | chain variable region |
| | GTSYSLTISRVEAEDAATYYCQQWSFNPPTFGG | |
| | GTKLEIK | |
| 84 | RASSSVNYMD | Anti-CD20 Leu16 scFv light |
| | | chain CDR1 |
| 85 | ATSNLAS | Anti-CD20 Leu16 scFv light |
| | | chain CDR2 |
| 86 | QQWSFNPPT | Anti-CD20 Leu16 scFv light |
| | | chain CDR3 |
| 87 | EVQLQQSGAELVKPGASVKMSCKASGYTFTSYN | Anti-CD20 Leu16 scFv |
| | MHWVKQTPGQGLEWIGAIYPGNGDTSYNQKF | heavy chain |
| | KGKATLTADKSSSTAYMQLSSLTSEDSADYYCAR | |
| | SNYYGSSYWFFDVWGAGTTVTVSS | |
| 88 | SYNMH | Anti-CD20 Leu16 scFv |
| | | heavy chain CDR1 |
| 89 | AIYPGNGDTSYNQKFKG | Anti-CD20 Leu16 scFv |
| | | heavy chain CDR2 |
| 144 | SNYYGSSYWFFDV | Anti-CD20 Leu16 scFv |
| | | heavy chain CDR3 |

In some embodiments, the hinge domain of the CD20 CAR comprises a CD80a hinge domain, for example, a human CD8α hinge domain. In some embodiments, the CD8α hinge domain comprises or consists of an amino acid sequence set forth in SEQ ID NO:50 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:50. In some embodiments, the hinge domain comprises a CD28 hinge domain, for example, a human CD28 hinge domain. In some embodiments, the CD28 hinge domain comprises or consists of an amino acid sequence set forth in SEQ ID NO:51 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:51. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:53 or SEQ ID NO:54, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:53 or SEQ ID NO:54. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:55 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:55.

In some embodiments, the transmembrane domain of the CD20 CAR comprises a CD8α transmembrane domain, for example, a human CD8α transmembrane domain. In some embodiments, the CD8α transmembrane domain comprises or consists of an amino acid sequence set forth in SEQ ID NO:56 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:56. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:57 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:57.

In some embodiments, the intracellular costimulatory domain of the CD20 CAR comprises a 4-1BB costimulatory domain, for example, a human 4-1BB costimulatory domain. In some embodiments, the 4-1BB costimulatory domain comprises or consists of an amino acid sequence set forth in SEQ ID NO:59 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:59. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO:60 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:60.

In some embodiments, the intracellular signaling domain of the CD20 CAR comprises a CD3 zeta (ζ) signaling domain, for example, a human CD3C signaling domain. In some embodiments, the CD3ζ signaling domain comprises or consists of an amino acid sequence set forth in SEQ ID NO:61 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:61.

In some embodiments, the CAR is a CD20 CAR, including, for example, a CD20 CAR comprising the CD20-specific scFv having sequences set forth in SEQ ID NO:82, the CD8α hinge domain of SEQ ID NO:50, the CD8α transmembrane domain of SEQ ID NO:56, the 4-1BB costimulatory domain of SEQ ID NO:59, the CD3ζ signaling domain of SEQ ID NO:61, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the CAR is a CD20 CAR, including, for example, a CD20 CAR comprising the CD20-specific scFv having sequences set forth in SEQ ID NO:82, the CD28 hinge domain of SEQ ID NO:51, the CD8α transmembrane domain of SEQ ID NO:56, the 4-1BB costimulatory domain of SEQ ID NO:59, the CD3ζ signaling domain of SEQ ID NO:61, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the CAR is a CD20 CAR, including, for example, a CD20 CAR comprising the CD20-specific scFv having sequences set forth in SEQ ID NO:82, the IgG4 hinge domain of SEQ ID NO:53 or SEQ ID NO:54, the CD8α transmembrane domain of SEQ ID NO:56, the 4-1BB costimulatory domain of SEQ ID NO:59, the CD3ζ signaling domain of SEQ ID NO:61, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the CAR is a CD20 CAR, including, for example, a CD20 CAR comprising the CD20-specific scFv having sequences set forth in SEQ ID NO:82, the CD8α hinge domain of SEQ ID NO:50, the CD28 transmembrane domain of SEQ ID NO:57, the 4-1BB costimulatory domain of SEQ ID NO:59, the CD3C signaling domain of SEQ ID NO:61, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the CAR is a CD20 CAR, including, for example, a CD20 CAR comprising the CD20-specific scFv having sequences set forth in SEQ ID NO:82, the CD28 hinge domain of SEQ ID NO:51, the CD28 transmembrane domain of SEQ ID NO:57, the 4-1BB costimulatory domain of SEQ JD NO:59, the CD3ζ signaling domain of SEQ JD NO:61, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the CAR is a CD20 CAR, including, for example, a CD20 CAR comprising the CD20-specific scFv having sequences set forth in SEQ ID NO:82, the IgG4 hinge domain of SEQ ID NO:53 or SEQ ID NO:54, the CD28 transmembrane domain of SEQ ID NO:57, the 4-1BB costimulatory domain of SEQ ID NO:59, the CD3ζ signaling domain of SEQ ID NO:61, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

### (c) CD22 CARs

In some embodiments, the CAR is a CD22 CAR (“CD22-CAR”). CD22, which is a transmembrane protein found mostly on the surface of mature B cells that functions as an inhibitory receptor for B cell receptor (BCR) signaling. CD22 is expressed in 60-70% of B cell lymphomas and leukemias (e.g., B-chronic lymphocytic leukemia, hairy cell leukemia, acute lymphocytic leukemia (ALL), and Burkitt's lymphoma) and is not present on the cell surface in early stages of B cell development or on stem cells. In some embodiments, the CD22 CAR may comprise a signal peptide, an extracellular binding domain that specifically binds CD22, a hinge domain, a transmembrane domain, an intracellular costimulatory domain, and/or an intracellular signaling domain in tandem.

In some embodiments, the signal peptide of the CD22 CAR comprises a CD8α signal peptide. In some embodiments, the CD8α signal peptide comprises or consists of an amino acid sequence set forth in SEQ ID NO:47 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:47. In some embodiments, the signal peptide comprises an IgK signal peptide. In some embodiments, the IgK signal peptide comprises or consists of an amino acid sequence set forth in SEQ ID NO:48 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:48. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:49 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:49.

In some embodiments, the extracellular binding domain of the CD22 CAR is specific to CD22, for example, human CD22. The extracellular binding domain of the CD22 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the CD22 CAR is derived from an antibody specific to CD22, including, for example, SM03, inotuzumab, epratuzumab, moxetumomab, and pinatuzumab. In any of these embodiments, the extracellular binding domain of the CD22 CAR can comprise or consist of the V_{H}, the V_{L}, and/or one or more CDRs of any of the antibodies.

In some embodiments, the extracellular binding domain of the CD22 CAR comprises an scFv derived from the m971 monoclonal antibody (m971), which comprises the heavy chain variable region (V_{H}) and the light chain variable region (V_{L}) of m971 connected by a linker. In some embodiments, the linker is a 3×G4S linker. In other embodiments, the Whitlow linker may be used instead. In some embodiments, the amino acid sequences of the entire m971-derived scFv (also referred to as m971 scFv) and its different portions are provided in Table 13 below. In some embodiments, the CD22-specific scFv comprises or consists of an amino acid sequence set forth in SEQ ID NO:90, 91, or 95, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:90, 91, or 95. In some embodiments, the CD22-specific scFv may comprise one or more CDRs having amino acid sequences set forth in SEQ ID NOs: 92-94 and 96-98. In some embodiments, the CD22-specific scFv may comprise a heavy chain with one or more CDRs having amino acid sequences set forth in SEQ ID NOs: 92-94. In some embodiments, the CD22-specific scFv may comprise a light chain with one or more CDRs having amino acid sequences set forth in SEQ ID NOs: 96-98. In any of these embodiments, the CD22-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD22 CAR comprises or consists of the one or more CDRs as described herein.

In some embodiments, the extracellular binding domain of the CD22 CAR comprises an scFv derived from m971-L7, which is an affinity matured variant of m971 with significantly improved CD22 binding affinity compared to the parental antibody m971 (improved from about 2 nM to less than 50 pM). In some embodiments, the scFv derived from m971-L7 comprises the V_{H} and the V_{L} of m971-L7 connected by a 3×G4S linker. In other embodiments, the Whitlow linker may be used instead. In some embodiments, the amino acid sequences of the entire m971-L7-derived scFv (also referred to as m971-L7 scFv) and its different portions are provided in Table 13 below. In some embodiments, the CD22-specific scFv comprises or consists of an amino acid sequence set forth in SEQ ID NO:99, 100, or 104, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:99, 100, or 104. In some embodiments, the CD22-specific scFv may comprise one or more CDRs having amino acid sequences set forth in SEQ ID NOs: 101-103 and 105-107. In some embodiments, the CD22-specific scFv may comprise a heavy chain with one or more CDRs having amino acid sequences set forth in SEQ ID NOs: 101-103. In some embodiments, the CD22-specific scFv may comprise a light chain with one or more CDRs having amino acid sequences set forth in SEQ ID NOs: 105-107. In any of these embodiments, the CD22-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD22 CAR comprises or consists of the one or more CDRs as described herein.

TABLE 13
Exemplary sequences of anti-CD22 scFv and components

| SEQ ID NO: | Amino Acid Sequence | Description |
|---|---|---|
| 90 | QVQLQQSGPGLVKPSQTLSLTCAISGDSVSSNSA | Anti-CD22 m971 scFv |
| | AWNWIRQSPSRGLEWLGRTYYRSKWYNDYAVS | entire sequence, with |
| | VKSRITINPDTSKNQFSLQLNSVTPEDTAVYYCAR | 3xG4S linker |
| | EVTGDLEDAFDIWGQGTMVTVSSGGGGSGGG | |
| | GSGGGGSDIQMTQSPSSLSASVGDRVTITCRAS | |
| | QTIWSYLNWYQQRPGKAPNLLIYAASSLQSGVP | |
| | SRFSGRGSGTDFTLTISSLQAEDFATYYCQQSYSI | |
| | PQTFGQGTKLEIK | |
| 91 | QVQLQQSGPGLVKPSQTLSLTCAISGDSVSSNSA | Anti-CD22 m971 scFv |
| | AWNWIRQSPSRGLEWLGRTYYRSKWYNDYAVS | heavy chain variable |
| | VKSRITINPDTSKNQFSLQLNSVTPEDTAVYYCAR | region |
| | EVTGDLEDAFDIWGQGTMVTVSS | |
| 92 | GDSVSSNSAA | Anti-CD22 m971 scFv |
| | | heavy chain CDR1 |
| 93 | TYYRSKWYN | Anti-CD22 m971 scFv |
| | | heavy chain CDR2 |
| 94 | AREVTGDLEDAFDI | Anti-CD22 m971 scFv |
| | | heavy chain CDR3 |
| 95 | DIQMTQSPSSLSASVGDRVTITCRASQTIWSYLN | Anti-CD22 m971 scFv light |
| | WYQQRPGKAPNLLIYAASSLQSGVPSRFSGRGS | chain |
| | GTDFTLTISSLQAEDFATYYCQQSYSIPQTFGQGT | |
| | KLEIK | |
| 96 | QTIWSY | Anti-CD22 m971 scFv light |
| | | chain CDR1 |
| 97 | AAS | Anti-CD22 m971 scFv light |
| | | chain CDR2 |
| 98 | QQSYSIPQT | Anti-CD22 m971 scFv light |
| | | chain CDR3 |
| 99 | QVQLQQSGPGMVKPSQTLSLTCAISGDSVSSNS | Anti-CD22 m971-L7 scFv |
| | VAWNWIRQSPSRGLEWLGRTYYRSTWYNDYA | entire sequence, with |
| | VSMKSRITINPDTNKNQFSLQLNSVTPEDTAVYY | 3xG4S linker |
| | CAREVTGDLEDAFDIWGQGTMVTVSSGGGGSG | |
| | GGGSGGGGSDIQMIQSPSSLSASVGDRVTITCR | |
| | ASQTIWSYLNWYRQRPGEAPNLLIYAASSLQSG | |
| | VPSRFSGRGSGTDFTLTISSLQAEDFATYYCQQSY | |
| | SIPQTFGQGTKLEIK | |
| 100 | QVQLQQSGPGMVKPSQTLSLTCAISGDSVSSNS | Anti-CD22 m971-L7 scFv |
| | VAWNWIRQSPSRGLEWLGRTYYRSTWYNDYA | heavy chain variable |
| | VSMKSRITINPDTNKNQFSLQLNSVTPEDTAVYY | region |
| | CAREVTGDLEDAFDIWGQGTMVTVSS | |
| 101 | GDSVSSNSVA | Anti-CD22 m971-L7 scFv |
| | | heavy chain CDR1 |
| 102 | TYYRSTWYN | Anti-CD22 m971-L7 scFv |
| | | heavy chain CDR2 |
| 103 | AREVTGDLEDAFDI | Anti-CD22 m971-L7 scFv |
| | | heavy chain CDR3 |
| 104 | DIQMIQSPSSLSASVGDRVTITCRASQTIWSYLN | Anti-CD22 m971-L7 scFv |
| | WYRQRPGEAPNLLIYAASSLQSGVPSRFSGRGS | light chain variable region |
| | GTDFTLTISSLQAEDFATYYCQQSYSIPQTFGQGT | |
| | KLEIK | |
| 105 | QTIWSY | Anti-CD22 m971-L7 scFv |
| | | light chain CDR1 |
| 106 | AAS | Anti-CD22 m971-L7 scFv |
| | | light chain CDR2 |
| 107 | QQSYSIPQT | Anti-CD22 m971-L7 scFv |
| | | light chain CDR3 |

In some embodiments, the extracellular binding domain of the CD22 CAR comprises immunotoxins HA22 or BL22. Immunotoxins BL22 and HA22 are therapeutic agents that comprise an scFv specific for CD22 fused to a bacterial toxin, and thus can bind to the surface of the cancer cells that express CD22 and kill the cancer cells. BL22 comprises a dsFv of an anti-CD22 antibody, RFB4, fused to a 38-kDa truncated form of Pseudomonas exotoxin A (Bang et al., Clin. Cancer Res., 11:1545-50 (2005)). HA22 (CAT8015, moxetumomab pasudotox) is a mutated, higher affinity version of BL22 (Ho et al., J. Biol. Chem., 280(1): 607-17 (2005)). Suitable sequences of antigen binding domains of HA22 and BL22 specific to CD22 are disclosed in, for example, U.S. Pat. Nos. 7,541,034; 7,355,012; and 7,982,011, which are hereby incorporated by reference in their entirety.

In some embodiments, the hinge domain of the CD22 CAR comprises a CD8α hinge domain, for example, a human CD8α hinge domain. In some embodiments, the CD8α hinge domain comprises or consists of an amino acid sequence set forth in SEQ ID NO:50 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:50. In some embodiments, the hinge domain comprises a CD28 hinge domain, for example, a human CD28 hinge domain. In some embodiments, the CD28 hinge domain comprises or consists of an amino acid sequence set forth in SEQ ID NO:51 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:51. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:53 or SEQ ID NO:54, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:53 or SEQ ID NO:54. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:55 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:55.

In some embodiments, the transmembrane domain of the CD22 CAR comprises a CD8α transmembrane domain, for example, a human CD8α transmembrane domain. In some embodiments, the CD8α transmembrane domain comprises or consists of an amino acid sequence set forth in SEQ ID NO:56 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:56. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:57 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:57.

In some embodiments, the intracellular costimulatory domain of the CD22 CAR comprises a 4-1BB costimulatory domain, for example, a human 4-1BB costimulatory domain. In some embodiments, the 4-1BB costimulatory domain comprises or consists of an amino acid sequence set forth in SEQ ID NO:59 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:59. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO:60 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:60.

In some embodiments, the intracellular signaling domain of the CD22 CAR comprises a CD3 zeta (ζ) signaling domain, for example, a human CD3ζ signaling domain. In some embodiments, the CD3ζ signaling domain comprises or consists of an amino acid sequence set forth in SEQ ID NO:61 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:61.

In some embodiments, the CAR is a CD22 CAR, including, for example, a CD22 CAR comprising the CD22-specific scFv having sequences set forth in SEQ ID NO:90 or SEQ ID NO:99, the CD8α hinge domain of SEQ ID NO:50, the CD8α transmembrane domain of SEQ ID NO:56, the 4-1BB costimulatory domain of SEQ ID NO:59, the CD3ζ signaling domain of SEQ ID NO:61, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the CAR is a CD22 CAR, including, for example, a CD22 CAR comprising the CD22-specific scFv having sequences set forth in SEQ ID NO:90 or SEQ ID NO:99, the CD28 hinge domain of SEQ ID NO:51, the CD8α transmembrane domain of SEQ ID NO:56, the 4-1BB costimulatory domain of SEQ ID NO:59, the CD3ζ signaling domain of SEQ JD NO:61, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the CAR is a CD22 CAR, including, for example, a CD22 CAR comprising the CD22-specific scFv having sequences set forth in SEQ ID NO:90 or SEQ ID NO:99, the IgG4 hinge domain of SEQ ID NO:53 or SEQ ID NO:54, the CD8α transmembrane domain of SEQ ID NO:56, the 4-1BB costimulatory domain of SEQ ID NO:59, the CD3ζ signaling domain of SEQ ID NO:61, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the CAR is a CD22 CAR, including, for example, a CD22 CAR comprising the CD22-specific scFv having sequences set forth in SEQ ID NO:90 or SEQ ID NO:99, the CD8α hinge domain of SEQ ID NO:50, the CD28 transmembrane domain of SEQ ID NO:57, the 4-1BB costimulatory domain of SEQ ID NO:59, the CD3ζ signaling domain of SEQ ID NO:61, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the CAR is a CD22 CAR, including, for example, a CD22 CAR comprising the CD22-specific scFv having sequences set forth in SEQ ID NO:90 or SEQ ID NO:99, the CD28 hinge domain of SEQ ID NO:51, the CD28 transmembrane domain of SEQ ID NO:57, the 4-1BB costimulatory domain of SEQ ID NO:59, the CD3ζ signaling domain of SEQ ID NO:61, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the CAR is a CD22 CAR, including, for example, a CD22 CAR comprising the CD22-specific scFv having sequences set forth in SEQ ID NO:90 or SEQ ID NO:99, the IgG4 hinge domain of SEQ ID NO:53 or SEQ ID NO:54, the CD28 transmembrane domain of SEQ ID NO:57, the 4-1BB costimulatory domain of SEQ ID NO:59, the CD3ζ signaling domain of SEQ JD NO:61, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

### (d) BCMA CARs

In some embodiments, the CAR is a BCMA CAR (“BCMA-CAR”). BCMA is a tumor necrosis family receptor (TNFR) member expressed on cells of the B cell lineage, with the highest expression on terminally differentiated B cells or mature B lymphocytes. BCMA is involved in mediating the survival of plasma cells for maintaining long-term humoral immunity. The expression of BCMA has been recently linked to a number of cancers, such as multiple myeloma, Hodgkin's and non-Hodgkin's lymphoma, various leukemias, and glioblastoma. In some embodiments, the BCMA CAR may comprise a signal peptide, an extracellular binding domain that specifically binds BCMA, a hinge domain, a transmembrane domain, an intracellular costimulatory domain, and/or an intracellular signaling domain in tandem.

In some embodiments, the signal peptide of the BCMA CAR comprises a CD8α signal peptide. In some embodiments, the CD8α signal peptide comprises or consists of an amino acid sequence set forth in SEQ ID NO:47 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:47. In some embodiments, the signal peptide comprises an IgK signal peptide. In some embodiments, the IgK signal peptide comprises or consists of an amino acid sequence set forth in SEQ ID NO:48 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:48. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:49 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:49.

In some embodiments, the extracellular binding domain of the BCMA CAR is specific to BCMA, for example, human BCMA. The extracellular binding domain of the BCMA CAR can be codon-optimized for expression in a host cell or to have variant sequences to increase functions of the extracellular binding domain.

In some embodiments, the extracellular binding domain comprises an immunogenically active portion of an immunoglobulin molecule, for example, an scFv. In some embodiments, the extracellular binding domain of the BCMA CAR is derived from an antibody specific to BCMA, including, for example, belantamab, elranatamab, teclistamab, LCAR-B38M, and ciltacabtagene. In any of these embodiments, the extracellular binding domain of the BCMA CAR can comprise or consist of the V_{H}, the V_{L}, and/or one or more CDRs of any of the antibodies.

In some embodiments, the extracellular binding domain of the BCMA CAR comprises an scFv derived from C11D5.3, a murine monoclonal antibody as described in Carpenter et al., Clin. Cancer Res. 19(8):2048-2060 (2013). See also PCT Application Publication No. WO2010/104949. The C11D5.3-derived scFv may comprise the heavy chain variable region (V_{H}) and the light chain variable region (V_{L}) of C11D5.3 connected by the Whitlow linker, the amino acid sequences of which is provided in Table 14 below. In some embodiments, the BCMA-specific extracellular binding domain comprises or consists of an amino acid sequence set forth in SEQ ID NO:108, 109, or 113, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:108, 109, or 113. In some embodiments, the BCMA-specific extracellular binding domain may comprise one or more CDRs having amino acid sequences set forth in SEQ ID NOs: 110-112 and 114-116. In some embodiments, the BCMA-specific extracellular binding domain may comprise a light chain with one or more CDRs having amino acid sequences set forth in SEQ ID NOs: 110-112. In some embodiments, the BCMA-specific extracellular binding domain may comprise a heavy chain with one or more CDRs having amino acid sequences set forth in SEQ ID NOs: 114-116. In any of these embodiments, the BCMA-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

In some embodiments, the extracellular binding domain of the BCMA CAR comprises an scFv derived from another murine monoclonal antibody, C12A3.2, as described in Carpenter et al., Clin. Cancer Res. 19(8):2048-2060 (2013) and PCT Application Publication No. WO2010/104949, the amino acid sequence of which is also provided in Table 14 below. In some embodiments, the BCMA-specific extracellular binding domain comprises or consists of an amino acid sequence set forth in SEQ ID NO:117, 118, or 122, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:117, 118, or 122. In some embodiments, the BCMA-specific extracellular binding domain may comprise one or more CDRs having amino acid sequences set forth in SEQ ID NOs: 119-121 and 123-125. In some embodiments, the BCMA-specific extracellular binding domain may comprise a light chain with one or more CDRs having amino acid sequences set forth in SEQ ID NOs: 119-121. In some embodiments, the BCMA-specific extracellular binding domain may comprise a heavy chain with one or more CDRs having amino acid sequences set forth in SEQ ID NOs: 123-135. In any of these embodiments, the BCMA-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

In some embodiments, the extracellular binding domain of the BCMA CAR comprises a murine monoclonal antibody with high specificity to human BCMA, referred to as BB2121 in Friedman et al., Hum. Gene Ther. 29(5):585-601 (2018)). See also, PCT Application Publication No. WO2012163805.

In some embodiments, the extracellular binding domain of the BCMA CAR comprises single variable fragments of two heavy chains (VHH) that can bind to two epitopes of BCMA as described in Zhao et al., J. Hematol. Oncol. 11(1):141 (2018), also referred to as LCAR-B38M. See also, PCT Application Publication No. WO2018/028647.

In some embodiments, the extracellular binding domain of the BCMA CAR comprises a fully human heavy-chain variable domain (FHVH) as described in Lam et al., Nat. Commun. 11(1):283 (2020), also referred to as FHVH33. See also, PCT Application Publication No. WO2019/006072. The amino acid sequences of FHVH33 and its CDRs are provided in Table 14 below. In some embodiments, the BCMA-specific extracellular binding domain comprises or consists of an amino acid sequence set forth in SEQ ID NO:126 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:126. In some embodiments, the BCMA-specific extracellular binding domain may comprise one or more CDRs having amino acid sequences set forth in SEQ ID NOs: 127-129. In any of these embodiments, the BCMA-specific extracellular binding domain may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

In some embodiments, the extracellular binding domain of the BCMA CAR comprises an scFv derived from CT103A (or CAR0085) as described in U.S. Pat. No. 11,026,975 B2, the amino acid sequence of which is provided in Table 14 below. In some embodiments, the BCMA-specific extracellular binding domain comprises or consists of an amino acid sequence set forth in SEQ ID NO:130, 131, or 135, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 130, 131, or 135. In some embodiments, the BCMA-specific extracellular binding domain may comprise one or more CDRs having amino acid sequences set forth in SEQ ID NOs: 132-134 and 136-138. In some embodiments, the BCMA-specific extracellular binding domain may comprise a light chain with one or more CDRs having amino acid sequences set forth in SEQ ID NOs: 132-134. In some embodiments, the BCMA-specific extracellular binding domain may comprise a heavy chain with one or more CDRs having amino acid sequences set forth in SEQ ID NOs: 136-138. In any of these embodiments, the BCMA-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

Additionally, CARs and binders directed to BCMA have been described in U.S. Application Publication Nos. 2020/0246381 A1 and 2020/0339699 A1, the entire contents of each of which are incorporated by reference herein.

TABLE 14
Exemplary sequences of anti-BCMA binder and components

| SEQ ID NO: | Amino Acid Sequence | Description |
|---|---|---|
| 108 | DIVLTQSPASLAMSLGKRATISCRASESVSVIGAH | Anti-BCMA C11D5.3 scFv |
| | LIHWYQQKPGQPPKLLIYLASNLETGVPARFSGS | entire sequence, with |
| | GSGTDFTLTIDPVEEDDVAIYSCLQSRIFPRTFGG | Whitlow linker |
| | GTKLEIKGSTSGSGKPGSGEGSTKGQIQLVQSGP | |
| | ELKKPGETVKISCKASGYTFTDYSINWVKRAPGK | |
| | GLKWMGWINTETREPAYAYDFRGRFAFSLETSA | |
| | STAYLQINNLKYEDTATYFCALDYSYAMDYWGQ | |
| | GTSVTVSS | |
| 109 | DIVLTQSPASLAMSLGKRATISCRASESVSVIGAH | Anti-BCMA C11D5.3 scFv |
| | LIHWYQQKPGQPPKLLIYLASNLETGVPARFSGS | light chain variable region |
| | GSGTDFTLTIDPVEEDDVAIYSCLQSRIFPRTFGG | |
| | GTKLEIK | |
| 110 | RASESVSVIGAHLIH | Anti-BCMA C11D5.3 scFv |
| | | light chain CDR1 |
| 111 | LASNLET | Anti-BCMA C11D5.3 scFv |
| | | light chain CDR2 |
| 112 | LQSRIFPRT | Anti-BCMA C11D5.3 scFv |
| | | light chain CDR3 |
| 113 | QIQLVQSGPELKKPGETVKISCKASGYTFTDYSIN | Anti-BCMA C11D5.3 scFv |
| | WVKRAPGKGLKWMGWINTETREPAYAYDFRG | heavy chain variable |
| | RFAFSLETSASTAYLQINNLKYEDTATYFCALDYSY | region |
| | AMDYWGQGTSVTVSS | |
| 114 | DYSIN | Anti-BCMA C11D5.3 scFv |
| | | heavy chain CDR1 |
| 115 | WINTETREPAYAYDFRG | Anti-BCMA C11D5.3 scFv |
| | | heavy chain CDR2 |
| 116 | DYSYAMDY | Anti-BCMA C11D5.3 scFv |
| | | heavy chain CDR3 |
| 117 | DIVLTQSPPSLAMSLGKRATISCRASESVTILGSHL | Anti-BCMA C12A3.2 scFv |
| | IYWYQQKPGQPPTLLIQLASNVQTGVPARFSGS | entire sequence, with |
| | GSRTDFTLTIDPVEEDDVAVYYCLQSRTIPRTFGG | Whitlow linker |
| | GTKLEIKGSTSGSGKPGSGEGSTKGQIQLVQSGP | |
| | ELKKPGETVKISCKASGYTFRHYSMNWVKQAPG | |
| | KGLKWMGRINTESGVPIYADDFKGRFAFSVETS | |
| | ASTAYLVINNLKDEDTASYFCSNDYLYSLDFWGQ | |
| | GTALTVSS | |
| 118 | DIVLTQSPPSLAMSLGKRATISCRASESVTILGSHL | Anti-BCMA C12A3.2 scFv |
| | IYWYQQKPGQPPTLLIQLASNVQTGVPARFSGS | light chain variable region |
| | GSRTDFTLTIDPVEEDDVAVYYCLQSRTIPRTFGG | |
| | GTKLEIK | |
| 119 | RASESVTILGSHLIY | Anti-BCMA C12A3.2 scFv |
| | | light chain CDR1 |
| 120 | LASNVQT | Anti-BCMA C12A3.2 scFv |
| | | light chain CDR2 |
| 121 | LQSRTIPRT | Anti-BCMA C12A3.2 scFv |
| | | light chain CDR3 |
| 122 | QIQLVQSGPELKKPGETVKISCKASGYTFRHYSM | Anti-BCMA C12A3.2 scFv |
| | NWVKQAPGKGLKWMGRINTESGVPIYADDFK | heavy chain variable |
| | GRFAFSVETSASTAYLVINNLKDEDTASYFCSNDY | region |
| | LYSLDFWGQGTALTVSS | |
| 123 | HYSMN | Anti-BCMA C12A3.2 scFv |
| | | heavy chain CDR1 |
| 124 | RINTESGVPIYADDFKG | Anti-BCMA C12A3.2 scFv |
| | | heavy chain CDR2 |
| 125 | DYLYSLDF | Anti-BCMA C12A3.2 scFv |
| | | heavy chain CDR3 |
| 126 | EVQLLESGGGLVQPGGSLRLSCAASGFTFSSYAM | Anti-BCMA FHVH33 entire |
| | SWVRQAPGKGLEWVSSISGSGDYIYYADSVKGR | sequence |
| | FTISRDISKNTLYLQMNSLRAEDTAVYYCAKEGT | |
| | GANSSLADYRGQGTLVTVSS | |
| 127 | GFTFSSYA | Anti-BCMA FHVH33 CDR1 |
| 128 | ISGSGDYI | Anti-BCMA FHVH33 CDR2 |
| 129 | AKEGTGANSSLADY | Anti-BCMA FHVH33 CDR3 |
| 130 | DIQMTQSPSSLSASVGDRVTITCRASQSISSYLN | Anti-BCMA CT103A scFv |
| | WYQQKPGKAPKLLIYAASSLQSGVPSRFSGSGSG | entire sequence, with |
| | TDFTLTISSLQPEDFATYYCQQKYDLLTFGGGTKV | Whitlow linker |
| | EIKGSTSGSGKPGSGEGSTKGQLQLQESGPGLVK | |
| | PSETLSLTCTVSGGSISSSSYYWGWIRQPPGKGLE | |
| | WIGSISYSGSTYYNPSLKSRVTISVDTSKNQFSLKL | |
| | SSVTAADTAVYYCARDRGDTILDVWGQGTMVT | |
| | VSS | |
| 131 | DIQMTQSPSSLSASVGDRVTITCRASQSISSYLN | Anti-BCMA CT103A scFv |
| | WYQQKPGKAPKLLIYAASSLQSGVPSRFSGSGSG | light chain variable region |
| | TDFTLTISSLQPEDFATYYCQQKYDLLTFGGGTKV | |
| | EIK | |
| 132 | QSISSY | Anti-BCMA CT103A scFv |
| | | light chain CDR1 |
| 133 | AAS | Anti-BCMA CT103A scFv |
| | | light chain CDR2 |
| 134 | QQKYDLLT | Anti-BCMA CT103A scFv |
| | | light chain CDR3 |
| 135 | QLQLQESGPGLVKPSETLSLTCTVSGGSISSSSYY | Anti-BCMA CT103A scFv |
| | WGWIRQPPGKGLEWIGSISYSGSTYYNPSLKSRV | heavy chain variable |
| | TISVDTSKNQFSLKLSSVTAADTAVYYCARDRGD | region |
| | TILDVWGQGTMVTVSS | |
| 136 | GGSISSSSYY | Anti-BCMA CT103A scFv |
| | | heavy chain CDR1 |
| 137 | ISYSGST | Anti-BCMA CT103A scFv |
| | | heavy chain CDR2 |
| 138 | ARDRGDTILDV | Anti-BCMA CT103A scFv |
| | | heavy chain CDR3 |

In some embodiments, the hinge domain of the BCMA CAR comprises a CD8α hinge domain, for example, a human CD8α hinge domain. In some embodiments, the CD8α hinge domain comprises or consists of an amino acid sequence set forth in SEQ ID NO:50 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:50. In some embodiments, the hinge domain comprises a CD28 hinge domain, for example, a human CD28 hinge domain. In some embodiments, the CD28 hinge domain comprises or consists of an amino acid sequence set forth in SEQ ID NO:51 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:51. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:53 or SEQ ID NO:54, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:53 or SEQ ID NO:54. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:55 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:55.

In some embodiments, the transmembrane domain of the BCMA CAR comprises a CD8α transmembrane domain, for example, a human CD8α transmembrane domain. In some embodiments, the CD8α transmembrane domain comprises or consists of an amino acid sequence set forth in SEQ ID NO:56 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:56. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:57 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:57.

In some embodiments, the intracellular costimulatory domain of the BCMA CAR comprises a 4-1BB costimulatory domain, for example, a human 4-1BB costimulatory domain. In some embodiments, the 4-1BB costimulatory domain comprises or consists of an amino acid sequence set forth in SEQ ID NO:59 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:59. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO:60 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:60.

In some embodiments, the intracellular signaling domain of the BCMA CAR comprises a CD3 zeta (ζ) signaling domain, for example, a human CD3ζ signaling domain. In some embodiments, the CD3ζ signaling domain comprises or consists of an amino acid sequence set forth in SEQ ID NO:61 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:61.

In some embodiments, the CAR is a BCMA CAR, including, for example, a BCMA CAR comprising any of the BCMA-specific extracellular binding domains as described, the CD8α hinge domain of SEQ ID NO:50, the CD8α transmembrane domain of SEQ ID NO:56, the 4-1BB costimulatory domain of SEQ ID NO:59, the CD3ζ signaling domain of SEQ ID NO:61, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the BCMA CAR may additionally comprise a signal peptide (e.g., a CD8α signal peptide) as described.

In some embodiments, the CAR is a BCMA CAR, including, for example, a BCMA CAR comprising any of the BCMA-specific extracellular binding domains as described, the CD8α hinge domain of SEQ ID NO:50, the CD8α transmembrane domain of SEQ ID NO:56, the CD28 costimulatory domain of SEQ ID NO:60, the CD3ζ signaling domain of SEQ ID NO:61, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the BCMA CAR may additionally comprise a signal peptide as described.

In some embodiments, the CAR is a BCMA CAR as set forth in SEQ ID NO:139 or is at least 80% identical (e.g., at least 80%, at least 85%, at least 90%, at least 95%, at least 96%, at least 97%, at least 98%, at least 99%, or 100% identical) to the nucleotide sequence set forth in SEQ ID NO:139 (see Table 15). The encoded BCMA CAR has a corresponding amino acid sequence set forth in SEQ ID NO:140 or is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:140, with the following components: CD8α signal peptide, CT103A scFv (V_{L}-Whitlow linker-V_{H}), CD8α hinge domain, CD8α transmembrane domain, 4-1BB costimulatory domain, and CD3ζ signaling domain.

In some embodiments, the CAR is a commercially available embodiment of BCMA CAR, including, for example, idecabtagene vicleucel (ide-cel, also called bb2121). In some embodiments, the CAR is idecabtagene vicleucel or portions thereof. Idecabtagene vicleucel comprises a BCMA CAR with the following components: the BB2121 binder, CD8α hinge domain, CD8α transmembrane domain, 4-1BB costimulatory domain, and CD3ζ signaling domain.

TABLE 15
Exemplary sequences of BCMA CARs

| SEQ ID NO: | Sequence | Description |
|---|---|---|
| 139 | atggccttaccagtgaccgccttgctcctgccgctggccttgctgctcca | Exemplary BCMA |
| | cgccgccaggccggacatccagatgacccagtctccatcctccctgtct | CAR nucleotide |
| | gcatctgtaggagacagagtcaccatcacttgccgggcaagtcagagc | sequence |
| | attagcagctatttaaattggtatcagcagaaaccagggaaagcccct | |
| | aagctcctgatctatgctgcatccagtttgcaaagtggggtcccatcaa | |
| | ggttcagtggcagtggatctgggacagatttcactctcaccatcagcag | |
| | tctgcaacctgaagattttgcaacttactactgtcagcaaaaatacgac | |
| | ctcctcacttttggcggagggaccaaggttgagatcaaaggcagcacc | |
| | agcggctccggcaagcctggctctggcgagggcagcacaaagggaca | |
| | gctgcagctgcaggagtcgggcccaggactggtgaagccttcggaga | |
| | ccctgtccctcacctgcactgtctctggtggctccatcagcagtagtagt | |
| | tactactggggctggatccgccagcccccagggaaggggctggagtg | |
| | gattgggagtatctcctatagtgggagcacctactacaacccgtccctc | |
| | aagagtcgagtcaccatatccgtagacacgtccaagaaccagttctcc | |
| | ctgaagctgagttctgtgaccgccgcagacacggcggtgtactactgc | |
| | gccagagatcgtggagacaccatactagacgtatggggtcagggtac | |
| | aatggtcaccgtcagctcattcgtgcccgtgttcctgcccgccaaaccta | |
| | ccaccacccctgcccctagacctcccaccccagccccaacaatcgcca | |
| | gccagcctctgtctctgcggcccgaagcctgtagacctgctgccggcgg | |
| | agccgtgcacaccagaggcctggacttcgcctgcgacatctacatctg | |
| | ggcccctctggccggcacctgtggcgtgctgctgctgagcctggtgatc | |
| | accctgtactgcaaccaccggaacaaacggggcagaaagaaactcct | |
| | gtatatattcaaacaaccatttatgagaccagtacaaactactcaaga | |
| | ggaagatggctgtagctgccgatttccagaagaagaagaaggaggat | |
| | gtgaactgagagtgaagttcagcagatccgccgacgcccctgcctacc | |
| | agcagggacagaaccagctgtacaacgagctgaacctgggcagacg | |
| | ggaagagtacgacgtgctggacaagcggagaggccgggaccccgag | |
| | atgggcggaaagcccagacggaagaacccccaggaaggcctgtata | |
| | acgaactgcagaaagacaagatggccgaggcctacagcgagatcgg | |
| | catgaagggcgagcggaggcgcggcaagggccacgatggcctgtacc | |
| | agggcctgagcaccgccaccaaggacacctacgacgccctgcacatg | |
| | caggccctgccccccaga | |
| 140 | MALPVTALLLPLALLLHAARPDIQMTQSPSSLSASVGDR | Exemplary BCMA |
| | VTITCRASQSISSYLNWYQQKPGKAPKLLIYAASSLQSGV | CAR amino acid |
| | PSRFSGSGSGTDFTLTISSLQPEDFATYYCQQKYDLLTFG | sequence |
| | GGTKVEIKGSTSGSGKPGSGEGSTKGQLQLQESGPGLVK | |
| | PSETLSLTCTVSGGSISSSSYYWGWIRQPPGKGLEWIGSI | |
| | SYSGSTYYNPSLKSRVTISVDTSKNQFSLKLSSVTAADTAV | |
| | YYCARDRGDTILDVWGQGTMVTVSSFVPVFLPAKPTTT | |
| | PAPRPPTPAPTIASQPLSLRPEACRPAAGGAVHTRGLDF | |
| | ACDIYIWAPLAGTCGVLLLSLVITLYCNHRNKRGRKKLLYI | |
| | FKQPFMRPVQTTQEEDGCSCRFPEEEEGGCELRVKFSRS | |
| | ADAPAYQQGQNQLYNELNLGRREEYDVLDKRRGRDPE | |
| | MGGKPRRKNPQEGLYNELQKDKMAEAYSEIGMKGERR | |
| | RGKGHDGLYQGLSTATKDTYDALHMQALPPR | |

### (e) GPRC5D Chimeric Antigen Receptor

In some embodiments, the antigen targeted by the antigen-binding domain is GPRC5D. In some aspects, the antigen-binding domain of the recombinant receptor, e.g., CAR, binds, such as specifically binds or specifically recognizes, a GPRC5D, such as a human GPRC5D. In some embodiments, the antibody or antigen binding fragment thereof comprises a V_{H} and a V_{L} derived from an antibody or an antibody fragment specific to GPRC5D as disclosed herein. In some embodiments, the antibody or antigen binding fragment thereof is a human antibody, e.g., as described in U.S. Patent Publication No. US 2016/0152723.

In some embodiments, the CAR is a GPRC5D CAR (“GPRC5D-CAR”). In some of these embodiments, a polycistronic vector comprises an expression cassette that contains a nucleotide sequence encoding a GPRC5D CAR or another CAR disclosed herein. GPRC5D is an orphan G protein-coupled receptor family member that is expressed on plasma cells. The expression of GPRC5D has been linked to multiple myeloma. In some embodiments, the GPRC5D CAR may comprise a signal peptide, an extracellular binding domain that specifically binds GPRC5D, a hinge domain, a transmembrane domain, an intracellular costimulatory domain, and/or an intracellular signaling domain in tandem.

In some embodiments, the GPRC5D CAR includes an antibody or antigen binding fragment thereof, a transmembrane domain, a co-stimulatory signaling domain, and a signaling domain. In some embodiments, the antibody or antigen binding fragment thereof is an anti-GPRC5D single-chain antibody fragment (scFv) or single-domain antibody fragment (sdAb). Table 20 provides several non-limiting exemplary sequences of full-length GPRC5D scFv and sdAb sequences. In some embodiments, the GPRC5D specific CAR includes an anti-GPRC5D single-chain antibody fragment (scFv) or single-domain antibody fragment (sdAb), a transmembrane domain such as one derived from human CD8α, a 4-1BB (CD137) co-stimulatory signaling domain, and a CD3ζ signaling domain.

TABLE 16
HCDRS in Kabat Numbering Scheme

| GPRC5D | H-CDR1 | | H-CDR2 | | H-CDR3 | |
|---|---|---|---|---|---|---|
| Binder | Sequence | SEQ ID NO: | Sequence | SEQ ID NO: | Sequence | SEQ ID NO: |
| 1 | GYTFTSYY | 504 | INPNSGGT | 507 | VRSKGRAARNYYYMDV | 510 |
| 2 | GFTFSSYA | 505 | ISGSGGST | 508 | ARDLYGYRYYYYGMDV | 511 |
| 3 | GDSVSSNSAA | 506 | TYYRSKWYN | 509 | ARAYSPSRLRWSRAAAFDI | 512 |

TABLE 17
LCDRS in Kabat Numbering Scheme

| GPRC5D | L-CDR1 | | L-CDR2 | | L-CDR3 | |
|---|---|---|---|---|---|---|
| Binder | Sequence | SEQ ID NO: | Sequence | SEQ ID NO: | Sequence | SEQ ID NO: |
| 1 | SVRTYY | 513 | GKN | 516 | NSRDSSANPV | 519 |
| 2 | SLRRYF | 514 | GKN | 517 | NSRDRSGTVV | 520 |
| 3 | SLRSYY | 515 | GKN | 518 | SSRDSSGNHLVV | 521 |

TABLE 18
VH Sequences

| GPRC5D | | SEQ |
|---|---|---|
| Binder | VH Sequence | ID NO: |
| 1 | QVQLVQSGAEVKKPGASVKVSCKASGYTFTSYY | 522 |
| | MHWVRQAPGQGLEWMGWINPNSGGTNYAQKFQG | |
| | RVTITADKSTSTAYMELNSLRAEDTAVYYCVRS | |
| | KGRAARNYYYMDVWGKGTTVTVSS | |
| 2 | EVQLVESGGGVVQPGRSLRLSCAASGFTFSSYA | 523 |
| | MSWVRQAPGKGLEWVSAISGSGGSTYYAGSVKG | |
| | RFTISRDNSKNTLYLQMNSLRAEDTAVYYCARD | |
| | LYGYRYYYYGMDVWGQGTMVTVSS | |
| 3 | QVQLQQSGPGQVKPSQTLSLTCAISGDSVSSNS | 524 |
| | AAWNWIRQSPSRGLEWLGRTYYRSKWYNDYAVS | |
| | VKSRITINPDTSKNQFSLQLNSVTPEDTAVYYC | |
| | ARAYSPSRLRWSRAAAFDIWGQGTTVTVSS | |

TABLE 19
VL Sequences

| GPRC5D | | SEQ |
|---|---|---|
| Binder | VH Sequence | ID NO: |
| 1 | SSELTQDPAASVALGQTVRITCQGDSVRTYYAG | 525 |
| | WYQQKPGQAPVLVIYGKNHRPSGIPDRFSGSTS | |
| | GNTASLTITGVQAEDEADYYCNSRDSSANPVFG | |
| | GGTKVTVL | |
| 2 | SSELTQDPAASVALGQTVKITCQGDSLRRYFAS | 526 |
| | WYQQKPGQAPTLVTYGKNRRPSGVPDRLSGSSS | |
| | GDTASLTITGAQAEDEGDYYCNSRDRSGTVVFG | |
| | GGTKLTVL | |
| 3 | SSELTQDPAVSVALGQTVRITCQGDSLRSYYAN | 527 |
| | WYQQKPGQAPILVNYGKNNRPSGIPDRFSGSSS | |
| | GKTASLTITGAQAEDEADYYCSSRDSSGNHLVV | |
| | FGGGTQLTVL | |

TABLE 20
Full GPRC5D Binder scFv and sdAb Sequences

| GPRC5D | | |
|---|---|---|
| Binder | scFv Sequence | SEQ ID NO: |
| 1 | SSELTQDPAASVALGQTVRITCQGDSVRTYYAGWYQQKPGQAPVLVIYGKNHRPSGIPDRF | 528 |
| | SGSTSGNTASLTITGVQAEDEADYYCNSRDSSANPVFGGGTKVTVLGGGGSGGGGSGGGGS | |
| | QVQLVQSGAEVKKPGASVKVSCKASGYTFTSYYMHWVRQAPGQGLEWMGWINPNSGGT | |
| | NYAQKFQGRVTITADKSTSTAYMELNSLRAEDTAVYYCVRSKGRAARNYYYMDVWGKGT | |
| | TVTVSS | |
| 2 | SSELTQDPAASVALGQTVKITCQGDSLRRYFASWYQQKPGQAPTLVTYGKNRRPSGVPDR | 529 |
| | LSGSSSGDTASLTITGAQAEDEGDYYCNSRDRSGTVVFGGGTKLTVLGGGGSGGGGGGGG | |
| | SEVQLVESGGGVVQPGRSLRLSCAASGFTFSSYAMSWVRQAPGKGLEWVSAISGSGGSTYY | |
| | AGSVKGRFTISRDNSKNTLYLQMNSLRAEDTAVYYCARDLYGYRYYYYGMDVWGQGTMV | |
| | TVSS | |
| 3 | SSELTQDPAVSVALGQTVRITCQGDSLRSYYANWYQQKPGQAPILVNYGKNNRPSGIPDRF | 530 |
| | SGSSSGKTASLTITGAQAEDEADYYCSSRDSSGNHLVVFGGGTQLTVLGGGGSGGGGSGGG | |
| | GSQVQLQQSGPGQVKPSQTLSLTCAISGDSVSSNSAAWNWIRQSPSRGLEWLGRTYYRSK | |
| | WYNDYAVSVKSRITINPDTSKNQFSLQLNSVTPEDTAVYYCARAYSPSRLRWSRAAAFDI | |
| | WGQGTTVTVSS | |

TABLE 21
Exemplary sequences of CAR components

| Component | Sequence | SEQ ID NO: |
|---|---|---|
| Extracellular | | |
| binding domain | | |
| GPRC5D Binder | SSELTQDPAASVALGQTVRITCQGDSVRTYYAGWYQQKPGQAPVLVIYGKNHRPSGI | 528 |
| 1 | PDRFSGSTSGNTASLTITGVQAEDEADYYCNSRDSSANPVFGGGTKVTVLGGGGSGG | |
| | GGSGGGGSQVQLVQSGAEVKKPGASVKVSCKASGYTFTSYYMHWVRQAPGQGLEW | |
| | MGWINPNSGGTNYAQKFQGRVTITADKSTSTAYMELNSLRAEDTAVYYCVRSKGRA | |
| | ARNYYYMDVWGKGTTVTVSS | |
| GPRC5D Binder | SSELTQDPAASVALGQTVKITCQGDSLRRYFASWYQQKPGQAPTLVTYGKNRRPSG | 529 |
| 2 | VPDRLSGSSSGDTASLTITGAQAEDEGDYYCNSRDRSGTVVFGGGTKLTVLGGGGSG | |
| | GGGSGGGGSEVQLVESGGGVVQPGRSLRLSCAASGFTFSSYAMSWVRQAPGKGLEW | |
| | VSAISGSGGSTYYAGSVKGRFTISRDNSKNTLYLQMNSLRAEDTAVYYCARDLYGYRY | |
| | YYYGMDVWGQGTMVTVSS | |
| GPRC5D Binder | SSELTQDPAVSVALGQTVRITCQGDSLRSYYANWYQQKPGQAPILVNYGKNNRPSGI | 530 |
| 3 | PDRFSGSSSGKTASLTITGAQAEDEADYYCSSRDSSGNHLVVFGGGTQLTVLGGGGS | |
| | GGGGSGGGGSQVQLQQSGPGQVKPSQTLSLTCAISGDSVSSNSAAWNWIRQSPSRG | |
| | LEWLGRTYYRSKWYNDYAVSVKSRITINPDTSKNQFSLQLNSVTPEDTAVYYCARA | |
| | YSPSRLRWSRAAAFDIWGQGTTVTVSS | |
| Spacer (e.g., | | |
| hinge) | | |
| IgG4 Hinge | ESKYGPPCPPCP | 531 |
| CD8 Hinge | TTTPAPRPPTPAPTIASQPLSLRPE | 532 |
| CD28 | IEVMYPPPYLDNEKSNGTIIHVKGKHLCPSPLFPGPSKP | 533 |
| Transmembrane | | |
| CD8 | ACRPAAGGAVHTRGLDFACDIYIWAPLAGTCGVLLLSLVITLYC | 534 |
| CD28 | FWVLVVVGGVLACYSLLVTVAFIIFWV | 535 |
| CD28 | MFWVLVVVGGVLACYSLLVTVAFIIFWV | 536 |
| Costimulatory | | |
| domain | | |
| CD28 | RSKRSRLLHSDYMNMTPRRPGPTRKHYQPYAPPRDFAAYRS | 537 |
| 4-1BB | KRGRKKLLYIFKQPFMRPVQTTQEEDGCSCRFPEEEEGGCEL | 538 |
| Primary | | |
| Signaling Domain | | |
| CD 3zeta | RVKFSRSADAPAYQQGQNQLYNELNLGRREEYDVLDKRRGRDPEMGGKPRRKNP | 539 |
| | QEGLYNELQKDKMAEAYSEIGMKGERRRGKGHDGLYQGLSTATKDTYDALHMQAL | |
| | PPR | |
| CD3zeta (Q > K) | RVKFSRSADAPAYKQGQNQLYNELNLGRREEYDVLDKRRGRDPEMGGKPRRKNP | 540 |
| | QEGLYNELQKDKMAEAYSEIGMKGERRRGKGHDGLYQGLSTATKDTYDALHMQAL | |
| | PPR | |

In some embodiments, the signal peptide of the GPRC5D CAR comprises a CD8α signal peptide. In some embodiments, the CD8α signal peptide comprises or consists of an amino acid sequence set forth in SEQ ID NO:47 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:47. In some embodiments, the signal peptide comprises an IgK signal peptide. In some embodiments, the IgK signal peptide comprises or consists of an amino acid sequence set forth in SEQ ID NO:48 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:48. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:49 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:49.

In some embodiments, the extracellular binding domain of the GPRC5D CAR is specific to GPRC5D, for example, human GPRC5D. The extracellular binding domain of the GPRC5D CAR is codon-optimized for expression in a host cell or to have variant sequences to increase functions of the extracellular binding domain.

In some embodiments, the extracellular binding domain comprises an immunogenically active portion of an immunoglobulin molecule, for example, an scFv. In some embodiments, the extracellular binding domain of the GPRC5D CAR is derived from an antibody specific to GPRC5D, including, for example, any one of the antibodies or antigen binding fragments thereof herein disclosed, and telquetamab. In any of these embodiments, the extracellular binding domain of the GPRC5D CAR can comprise or consist of the V_{H}, the V_{L}, and/or one or more CDRs of any of the antibodies or antigen binding fragments thereof disclosed herein.

In some embodiments, the extracellular binding domain of the GPRC5D CAR comprises an scFv. The scFv may comprise the heavy chain variable region (V_{H}) and the light chain variable region (V_{L}) connected by a (G4S)3 linker or by a Whitlow linker. In some embodiments, the GPRC5D-specific extracellular binding domain comprises or consists of an amino acid sequence set forth in SEQ ID NO: 528, 529, or 530, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 528, 529, or 530, set forth in Table 20. In some embodiments, the GPRC5D-specific extracellular binding domain may comprise one or more heavy chain CDRs having amino acid sequences set forth in Table 16 and one or more light chain CDRs having amino acid sequences set forth in Table 17. In some embodiments, the GPRC5D-specific extracellular binding domain may comprise a heavy chain having amino acid sequences set forth in Table 18. In some embodiments, the GPRC5D-specific extracellular binding domain may comprise a light chain having amino acid sequences set forth in Table 19. In any of these embodiments, the GPRC5D-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In any of these embodiments, the GPRC5D-specific scFv may comprise one or more heavy chains (V_{H}) comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In any of these embodiments, the GPRC5D-specific scFv may comprise one or more light chains (V_{L}) comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the GPRC5D CAR comprises or consists of the one or more CDRs as described herein.

In some embodiments, the extracellular binding domain of the GPRC5D CAR comprises single variable fragments of a heavy chain (V_{H}) that can bind to an epitopes of GPRC5D.

In some embodiments, the hinge domain of the GPRC5D CAR comprises a CD8α hinge domain, for example, a human CD8α hinge domain. In some embodiments, the CD80 hinge domain comprises or consists of an amino acid sequence set forth in SEQ ID NO:50 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:50. In some embodiments, the hinge domain comprises a CD28 hinge domain, for example, a human CD28 hinge domain. In some embodiments, the CD28 hinge domain comprises or consists of an amino acid sequence set forth in SEQ ID NO:51 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:51. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:53 or SEQ ID NO:54, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:53 or SEQ ID NO:54. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:55 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:55. Non-limiting exemplary sequences of hinge domains are set forth in Table 4.

In some embodiments, the transmembrane domain comprises one selected from a group that includes a transmembrane region of TCRα, TCR3, TCRζ, CD3ε, CD3γ, CD3δ, CD3ζ, CD4, CDS, CD8α, CD8β, CD9, CD16, CD28, CD45, CD22, CD33, CD34, CD37, CD40, CD40L/CD154, CD45, CD64, CD80, CD86, OX40/CD134, 4-1BB/CD137, CD154, FcεRI γ, VEGFR2, FAS, FGFR2B, and functional variant thereof.

In some embodiments, the transmembrane domain of the GPRC5D CAR comprises a CD8α transmembrane domain, for example, a human CD8α transmembrane domain. In some embodiments, the CD8α transmembrane domain comprises or consists of an amino acid sequence set forth in SEQ ID NO:56 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:56. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:57 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:57. Non-limiting exemplary sequences of transmembrane domains are set forth in Table 5.

In some embodiments, the signaling domain(s) of the CAR comprises a costimulatory domain(s). For instance, a signaling domain can contain a costimulatory domain. Or, a signaling domain can contain one or more costimulatory domains. In some embodiments, the signaling domain comprises a costimulatory domain. In other embodiments, the signaling domains comprise costimulatory domains. In some embodiments, when the CAR comprises two or more costimulatory domains, two costimulatory domains are not the same. In some embodiments, the costimulatory domains comprise two costimulatory domains that are not the same. In some embodiments, the costimulatory domain enhances cytokine production, CAR-T cell proliferation, and/or CAR-T cell persistence during T cell activation. In some embodiments, the costimulatory domains enhance cytokine production, CAR-T cell proliferation, and/or CAR-T cell persistence during T cell activation.

In some embodiments, the intracellular costimulatory domain of the GPRC5D CAR comprises a 4-1BB costimulatory domain, for example, a human 4-1BB costimulatory domain. In some embodiments, the 4-1BB costimulatory domain comprises or consists of an amino acid sequence set forth in SEQ ID NO:59 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:59. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO:60 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:60. Non-limiting exemplary sequences of intracellular costimulatory and/or signaling domains are set forth in Table 6.

In some embodiments, the intracellular signaling domain of the GPRC5D CAR comprises a CD3 zeta (ζ) signaling domain, for example, a human CD3ζ signaling domain. In some embodiments, the CD3 signaling domain comprises or consists of an amino acid sequence set forth in SEQ ID NO:61 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:61.

In some embodiments, the CAR is a GPRC5D CAR, including, for example, any of the GPRC5D-specific extracellular binding domains as described, the CD8α hinge domain of SEQ ID NO: 532, the CD8α transmembrane domain of SEQ ID NO: 534, the 4-1BB costimulatory domain of SEQ ID NO: 538, the CD3ζ signaling domain of SEQ ID NO: 539, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the GPRC5D CAR may additionally comprise a signal peptide (e.g., a CD8α signal peptide) as described.

In some embodiments, the CAR is a GPRC5D CAR, including, for example, a GPRC5D CAR comprising any of the GPRC5D-specific extracellular binding domains as described, the CD8α hinge domain of SEQ ID NO: 532, the CD8α transmembrane domain of SEQ ID NO: 534, the CD28 costimulatory domain of SEQ ID NO: 537, the CD3ζ signaling domain of SEQ ID NO: 539, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the GPRC5D CAR may additionally comprise a signal peptide as described.

In some embodiments, the CAR binds to CD19. In some embodiments, the CAR binds to CD22. In some embodiments, the CAR binds to CD20. In some embodiments, the CAR binds to BCMA. In some embodiments, the CAR binds to an EBV antigen. In some embodiments, the CAR binds to CD27. In some embodiments, the CAR binds to CD30. In some embodiments, the CAR binds to CD19 and CD20. In some embodiments, the CAR binds to CD19 and CD22. In some embodiments, the CAR binds to CD19 and CD27. In some embodiments, the CAR binds to EBNA1. In some embodiments, the CAR binds to EBNA3A. In some embodiments, the CAR binds to BRLF1. In some embodiments, the CAR binds to BALF4. In some embodiments, the CAR binds to EBNA3C. In some embodiments, the CAR binds to LMPL. In some embodiments, the CAR binds to LMP2. In some embodiments, the CAR binds to LMP2A. In some embodiments, the CAR binds to LMP2B. In some embodiments, the CAR binds to BZLF1. In some embodiments, the CAR binds to BMLF1. In some embodiments, the CAR binds to gp350. In some embodiments, the CAR binds to gH/gL. In some embodiments, the CAR binds to EBNA1 and LMPL. In some embodiments, the CAR binds to EBNA1 and LMP2A. In some embodiments, the CAR binds to EBNA1, LMP1 and LMP2A. In some embodiments, the CAR binds to LMP, BARF1 and EBNA1. In some embodiments, the CAR binds to CD19 and an EBV antigen. In some embodiments, the CAR binds to CD20 and an EBV antigen. In some embodiments, the CAR binds to CD22 and an EBV antigen. In some embodiments, the CAR is selected from the group consisting of a first generation CAR, a second generation CAR, a third generation CAR, and a fourth generation CAR. In some embodiments, the CAR includes a single binding domain that binds to a single target antigen. In some embodiments, the CAR includes a single binding domain that binds to more than one target antigen, e.g., 2, 3, or more target antigens. In some embodiments, the CAR includes two binding domains such that each binding domain binds to a different target antigens. In some embodiments, the CAR includes two binding domains such that each binding domain binds to the same target antigen. Detailed descriptions of exemplary CARs including CD19-specific, CD20-specific and CD19/CD20-bispecific CARs can be found in WO2012/079000, WO2016/149578 and WO2020/014482, the disclosures including the sequence listings and figures are incorporated herein by reference in their entirety. In some embodiments, the CAR includes two binding domains such that each binding domain binds to the same target antigen. Detailed descriptions of exemplary CARs including CD19-specific, CD22-specific and CD19/CD22-bispecific CARs can be found in WO2012/079000, WO2016/149578 and WO2020/014482, the disclosures including the sequence listings and figures are incorporated herein by reference in their entirety. Detailed descriptions of exemplary CARs, TCRs or scFvs including CD27-specific, CD30-specific, EBNA1-specific, EBNA3C-specific, LMP1-specific, LMP2-specific, LMP2A-specific, gp350-specific CARs, gH/gL-specific CARs can be found in EP 2 520 589, U.S. Pat. No. 9,403,914, U.S. Ser. No. 11/180,566, US 2021/0009706, EP 2 558 498 B1, WO 2021/222929, US 2021/10206863, WO 2015/199617A1, WO 2012/109659A1, U.S. Pat. No. 7,786,269B2, WO 2021/211455A1, US 2016/0199479, WO2019/201995A1, and U.S. Ser. No. 11/116,835, the disclosures including the sequence listings and figures are incorporated herein by reference in their entireties. In some embodiments, the CAR includes two binding domains such that each binding domain binds to the same target antigen.

In some embodiments, the CD19 specific CAR includes an anti-CD19 single-chain antibody fragment (scFv), a transmembrane domain such as one derived from human CD8α, a 4-1BB (CD137) co-stimulatory signaling domain, and a CD3ζ signaling domain. In some embodiments, the CD20 specific CAR includes an anti-CD20 scFv, a transmembrane domain such as one derived from human CD8α, a 4-1BB (CD137) co-stimulatory signaling domain, and a CD3ζ signaling domain. In some embodiments, the CD19/CD20-bispecific CAR includes an anti-CD19 scFv, an anti-CD20 scFv, a transmembrane domain such as one derived from human CD8α, a 4-1BB (CD137) co-stimulatory signaling domain, and a CD3ζ signaling domain. In some embodiments, the CD22 specific CAR includes an anti-CD22 scFv, a transmembrane domain such as one derived from human CD8α, a 4-1BB (CD137) co-stimulatory signaling domain, and a CD3ζ signaling domain. In some embodiments, the CD19/CD22-bispecific CAR includes an anti-CD19 scFv, an anti-CD22 scFv, a transmembrane domain such as one derived from human CD8α, a 4-1BB (CD137) co-stimulatory signaling domain, and a CD3ζ signaling domain. In some embodiments, the EBNA1 specific CAR includes an anti-EBNA1 scFv, a transmembrane domain such as one derived from human CD8α, a 4-1BB (CD137) co-stimulatory signaling domain, and a CD3C signaling domain. In some embodiments, the EBNA3A CAR includes an anti-EBNA3A scFv, a transmembrane domain such as one derived from human CD8α, a 4-1BB (CD137) co-stimulatory signaling domain, and a CD3ζ signaling domain. In some embodiments, the EBNA3C CAR includes an anti-EBNA3C scFv, a transmembrane domain such as one derived from human CD8α, a 4-1BB (CD137) co-stimulatory signaling domain, and a CD3ζ signaling domain. In some embodiments, the LMP1 specific CAR includes an anti-LMP1 scFv, a transmembrane domain such as one derived from human CD8α, a 4-1BB (CD137) co-stimulatory signaling domain, and a CD3ζ signaling domain. In some embodiments, the LMP2 specific CAR includes an anti-LMP2 scFv, a transmembrane domain such as one derived from human CD8α, a 4-1BB (CD137) co-stimulatory signaling domain, and a CD3ζ signaling domain. In some embodiments, the LMP2A CAR includes an anti-LMP2A scFv, a transmembrane domain such as one derived from human CD8α, a 4-1BB (CD137) co-stimulatory signaling domain, and a CD3C signaling domain. In some embodiments, the BZLF1 CAR includes an anti-BZLF1 scFv, a transmembrane domain such as one derived from human CD8α, a 4-1BB (CD137) co-stimulatory signaling domain, and a CD3ζ signaling domain. In some embodiments, the BMLF1 CAR includes an anti-BMLF1 scFv, a transmembrane domain such as one derived from human CD8α, a 4-1BB (CD137) co-stimulatory signaling domain, and a CD3ζ signaling domain. In some embodiments, the gp350 CAR includes an anti-gp350 scFv, a transmembrane domain such as one derived from human CD8α, a 4-1BB (CD137) co-stimulatory signaling domain, and a CD3ζ signaling domain. In some embodiments, the gH/gL specific CAR includes an anti-gH/gL scFv, a transmembrane domain such as one derived from human CD8α, a 4-1BB (CD137) co-stimulatory signaling domain, and a CD3ζ signaling domain.

In some embodiments, the CAR comprises a commercial CAR construct carried by a T cell. Non-limiting examples of commercial CAR-T cell based therapies include brexucabtagene autoleucel (TECARTUS®), axicabtagene ciloleucel (YESCARTA®), idecabtagene vicleucel (ABECMA®), lisocabtagene maraleucel (BREYANZI®), tisagenlecleucel (KYMRIAH®), Descartes-08 and Descartes-11 from Cartesian Therapeutics, CTL119 from Novartis, β-BMCA-101 from Poseida Therapeutics, PBCAR19B and PBCAR269A from Precision Biosciences, FT819 from Fate Therapeutics, and CYAD-211 from Clyad Oncology.

In some embodiments, a hypoimmunogenic cell described herein comprises a polynucleotide encoding a chimeric antigen receptor (CAR) comprising an antigen binding domain. In some embodiments, a hypoimmunogenic cell described herein comprises a chimeric antigen receptor (CAR) comprising an antigen binding domain. In some embodiments, the polynucleotide is or comprises a chimeric antigen receptor (CAR) comprising an antigen binding domain. In some embodiments, the CAR is or comprises a first generation CAR comprising an antigen binding domain, a transmembrane domain, and at least one signaling domain (e.g., one, two or three signaling domains). In some embodiments, the CAR comprises a second generation CAR comprising an antigen binding domain, a transmembrane domain, and at least two signaling domains. In some embodiments, the CAR comprises a third generation CAR comprising an antigen binding domain, a transmembrane domain, and at least three signaling domains. In some embodiments, a fourth generation CAR comprising an antigen binding domain, a transmembrane domain, three or four signaling domains, and a domain which upon successful signaling of the CAR induces expression of a cytokine gene. In some embodiments, the antigen binding domain is or comprises an antibody, an antibody fragment, an scFv or a Fab.

(f) Chimeric B-cell autoantibody receptor (BAR)

In some embodiments, a transgene can encode a chimeric B-cell autoantibody receptor (BAR). A BAR recognizes and binds to certain antibody-expressing B cells. In some embodiments, a BAR comprises an antigen. An antigen of a BAR can be bound by neutralizing antibodies. The neutralizing antibodies may be undesirable because they can block or inhibit an effect or function of antigen to which they bind. For example, hemophilia patients can receive therapeutic factor VIII (FVIII) as part of their treatment. However, a patient's body may develop an immune response against the FVIII, including the production of anti-FVIII antibodies from B cells. When the patient produces anti-FVIII antibodies that bind to FVIII, FVIII is not able to perform its therapeutic functions. Accordingly, it may be beneficial to remove the anti-FVIII antibodies and/or the B-cells producing those antibodies from the patient. A BAR, which includes an FVIII antigen, can be used for this purpose.

In some embodiments, a BAR comprises a transmembrane domain. In some embodiments, a BAR comprises a signaling domain. In some embodiments, a BAR comprises one or more signaling domains.

In some embodiments, a BAR comprises an antigen, a transmembrane domain, and a signaling domain. In some embodiments, a BAR comprises an antigen, a transmembrane domain, and one or more signaling domains.

A BAR can be expressed by, e.g., a hypoimmunogenic T-cell. BAR T-cells can recognize and can bind target select antibodies and/or the B cells producing those antibodies. Once a BAR T-cell binds a target antibody, the BAR T-cell can destroy the antibodies and/or the B cells producing those antibodies. In some embodiments, a BAR T-cell is a BAR T-cell (Treg), e.g., a regulatory T-cell (Treg) comprising a BAR.

A BAR can be expressed by, e.g., a hypoimmunogenic NK-cell BAR. NK-cells can recognize and can bind target select antibodies and/or the B cells producing those antibodies. Once a BAR NK-cell binds a target antibody, the BAR NK-cell can destroy the antibodies and/or the B cells producing those antibodies.

### 2. Tolerogenic Factors

In some embodiments, described herein is an engineered lentiviral vector, wherein the first genome and/or the second genome further includes a transgene encoding a tolerogenic factor. In some embodiments, described herein is an engineered lentiviral vector, wherein the first transgene includes a biscistronic or multicistronic expression cassette encoding the first CAR and a tolerogenic factor and/or the second transgene includes a biscistronic or multicistronic expression cassette encoding the second CAR and a tolerogenic factor. In some embodiments, a transgene can encode a tolerogenic factor. In some embodiments, a tolerogenic factor is CD47, DUX4, CD24, CD27, CD35, CD46, CD55, CD59, CD200, HLA-C, HLA-E, HLA-E heavy chain, HLA-G, PD-L1, IDO1, CTLA4-Ig, C1-Inhibitor, IL-10, IL-35, FasL, CCL21, CCL22, Mfge8, CD16, CD52, H2-M3, CD16 Fe receptor, IL15-RF, and Serpinb9, A20/TNFAIP3, CD39, CR1, HLA-F, IL15-RF, MANF, or a SIRPα-binding polypeptide (e.g. a SIRPα engager). In some embodiments, a tolerogenic factor is CD47. In some embodiments, described herein is an engineered lentiviral vector, wherein the first genome includes a first tolerogenic factor and the second genome includes a second tolerogenic factor, and the first and second tolerogenic factors are different. In some embodiments, described herein is an engineered lentiviral vector, wherein the first tolerogenic factor and the second tolerogenic factor are selected from the group including or consisting of CD47, a SIRPα engager, A20/TNFAIP3, B2M-HLA-E, CD16, CD16 Fc receptor, CD24, CD27, CD35, CD39, CD46, CD47, CD52, CD55, CD59, CD64, CD200, CCL21, CCL22, CTLA4-Ig, C1 inhibitor, CR1, DUX4, FASL, HLA-C, HLA-E, HLA-E heavy chain, HLA-F, HLA-G, H2-M3, IDO1, IL-10, IL15-RF, IL-35, IL-39, MANF, Mfge8, PD-L1, or Serpinb9. In some embodiments, described herein is an engineered lentiviral vector, wherein the first genome includes a tolerogenic factor and the second genome does not include a tolerogenic factor.

In some embodiments, the present disclosure provides a method for altering a cell genome to express one or more tolerogenic factors. In some embodiments, the cell is genetically modified to comprise an integrated exogenous polynucleotide encoding one or more tolerogenic factors lentiviral-mediated integration into the genome of the cell. In some embodiments, the cell is genetically modified to comprise an integrated exogenous polynucleotide encoding one or more tolerogenic factors using homology-directed repair. In some instances, the cell expresses a nucleotide sequence encoding a tolerogenic factor polypeptide such that the nucleotide sequence is inserted into at least one allele of a safe harbor or target locus. In some instances, the cell expresses a nucleotide sequence encoding a tolerogenic factor wherein the nucleotide sequence is inserted into at least one allele of an AAVS1 locus. In some instances, the cell expresses a nucleotide sequence encoding a tolerogenic factor wherein the nucleotide sequence is inserted into at least one allele of a CCR5 locus. In some instances, the cell expresses a nucleotide sequence encoding a tolerogenic factor wherein the nucleotide sequence is inserted into at least one allele of a safe harbor or target gene locus, such as, but not limited to, a CCR5 gene locus, a CXCR4 gene locus, a PPP1R12C gene locus, an albumin gene locus, a SHS231 gene locus, a CLYBL gene locus, a Rosa gene locus, an F3 (CD142) gene locus, a MICA gene locus, a MICB gene locus, a LRP1 (CD91) gene locus, a HMGB1 gene locus, an ABO gene locus, an RHD gene locus, a FUT1 locus, and a KDM5D gene locus. In some instances, the cell expresses a nucleotide sequence encoding a tolerogenic factor wherein the nucleotide sequence is inserted into at least one allele of a TRAC locus.

### (i) CD47

In some embodiments, described herein is an engineered lentiviral vector comprising a transgene that encodes a CD47. CD47 is a leukocyte surface antigen and has a role in cell adhesion and modulation of integrins. It is expressed on the surface of a cell and signals to circulating macrophages not to eat the cell.

In some embodiments, the transgene comprises a nucleotide sequence encoding a CD47 polypeptide has at least 95% sequence identity (e.g., 95%, 96%, 97%, 98%, 99%, or more) to an amino acid sequence as set forth in NCBI Ref. Sequence Nos. NP_001768.1 and NP_942088.1. In some embodiments, the transgene comprises a nucleotide sequence encoding a CD47 polypeptide having an amino acid sequence as set forth in NCBI Ref. Sequence Nos. NP_001768.1 and NP_942088.1. In some embodiments, the transgene comprises a nucleotide sequence for CD47 having at least 85% sequence identity (e.g., 85%, 86%, 87%, 88%, 89%, 90%, 91%, 92%, 93%, 94%, 95%, 96%, 97%, 98%, 99%, or more) to the sequence set forth in NCBI Ref. Nos. NM_001777.3 and NM_198793.2. In some embodiments, the transgene comprises a nucleotide sequence for CD47 as set forth in NCBI Ref. Sequence Nos. NM_001777.3 and NM_198793.2. In some embodiments, the nucleotide sequence encoding a CD47 polynucleotide is a codon optimized sequence. In some embodiments, the nucleotide sequence encoding a CD47 polynucleotide is a human codon optimized sequence.

In some embodiments, the transgene encodes a CD47 polypeptide having at least 95% sequence identity (e.g., 95%, 96%, 97%, 98%, 99%, or more) to an amino acid sequence as set forth in NCBI Ref. Sequence Nos. NP_001768.1 and NP_942088.1. In some embodiments, the transgene encodes a CD47 polypeptide having an amino acid sequence as set forth in NCBI Ref. Sequence Nos. NP_001768.1 and NP_942088.1.

Exemplary amino acid sequences of human CD47 with a signal sequence and without a signal sequence are provided in Table 22.

TABLE 22
Exemplary sequences of CD47

| SEQ ID | | |
|---|---|---|
| NO: | Sequence | Description |
| 541 | MWPLVAALLLGSACCGSAQLLFNKTKSVEFTFCNDTVVIPCFVTNMEAQN | Amino acid |
| | TTEVYVKWKFKGRDIYTFDGALNKSTVPTDFSSAKIEVSQLLKGDASLKM | sequence encoded |
| | DKSDAVSHTGNYTCEVTELTREGETIIELKYRVVSWFSPNENILIVIFPIFAIL | by CDS of |
| | LFWGQFGIKTLKYRSGGMDEKTIALLVAGLVITVIVIVGAILFVPGEYSLKNA | NM_001777.4 |
| | TGLGLIVTSTGILILLHYYVFSTAIGLTSFVIAILVIQVIAYILAVVGLSLCIAACI | |
| | PMHGPLLISGLSILALAQLLGLVYMKFVASNQKTIQPPRKAVEEPLNAFKES | |
| | KGMMNDE | |
| 542 | MWPLVAALLLGSACCGSAQLLFNKTKSVEFTFCNDTVVIPCFVTNMEAQN | Amino acid |
| | TTEVYVKWKFKGRDIYTFDGALNKSTVPTDFSSAKIEVSQLLKGDASLKM | sequence encoded |
| | DKSDAVSHTGNYTCEVTELTREGETIIELKYRVVSWFSPNENILIVIFPIFAIL | by CDS of |
| | LFWGQFGIKTLKYRSGGMDEKTIALLVAGLVITVIVIVGAILFVPGEYSLKNA | NM_198793.2 |
| | TGLGLIVTSTGILILLHYYVFSTAIGLTSFVIAILVIQVIAYILAVVGLSLCIAACI | |
| | PMHGPLLISGLSILALAQLLGLVYMKFVASNQKTIQPPRNN | |
| 543 | atgtggcccctggtagcggcgctgttgctgggctcggcgtgctgcggatcagctca | Nucleotide |
| | gctactatttaataaaacaaaatctgtagaattcacgttttgtaatgacactgtcgtc | sequence of |
| | attccatgctttgttactaatatggaggcacaaaacactactgaagtatacgtaaag | NM_001777.4 CDS |
| | tggaaatttaaaggaagagatatttacacctttgatggagctctaaacaagtccac | (nts 124-1095) |
| | tgtccccactgactttagtagtgcaaaaattgaagtctcacaattactaaaaggag | |
| | atgcctctttgaagatggataagagtgatgctgtctcacacacaggaaactacactt | |
| | gtgaagtaacagaattaaccagagaaggtgaaacgatcatcgagctaaaatatcg | |
| | tgttgtttcatggttttctccaaatgaaaatattcttattgttattttcccaatttttgct | |
| | atactcctgttctggggacagtttggtattaaaacacttaaatatagatccggtggt | |
| | atggatgagaaaacaattgctttacttgttgctggactagtgatcactgtcattgtca | |
| | ttgttggagccattcttttcgtcccaggtgaatattcattaaagaatgctactggcctt | |
| | ggtttaattgtgacttctacagggatattaatattacttcactactatgtgtttagtac | |
| | agcgattggattaacctccttcgtcattgccatattggttattcaggtgatagcctat | |
| | atcctcgctgtggttggactgagtctctgtattgcggcgtgtataccaatgcatggcc | |
| | ctcttctgatttcaggtttgagtatcttagctctagcacaattacttggactagtttat | |
| | atgaaatttgtggcttccaatcagaagactatacaacctcctaggaaagctgtaga | |
| | ggaaccccttaatgcattcaaagaatcaaaaggaatgatgaatgatgaataa | |
| 544 | atgtggcccctggtagcggcgctgttgctgggctcggcgtgctgcggatcagctcagctactatttaa | Nucleotide |
| | taaaacaaaatctgtagaattcacgttttgtaatgacactgtcgtcattccatgctttgttactaatatg | sequence of |
| | gaggcacaaaacactactgaagtatacgtaaagtggaaatttaaaggaagagatatttacacctttg | NM_198793.2 CDS |
| | atggagctctaaacaagtccactgtccccactgactttagtagtgcaaaaattgaagtctcacaatta | (nts 181-1098) |
| | ctaaaaggagatgcctctttgaagatggataagagtgatgctgtctcacacacaggaaactacactt | |
| | gtgaagtaacagaattaaccagagaaggtgaaacgatcatcgagctaaaatatcgtgttgtttcatg | |
| | gttttctccaaatgaaaatattcttattgttattttcccaatttttgctatactcctgttctggggacagttt | |
| | ggtattaaaacacttaaatatagatccggtggtatggatgagaaaacaattgctttacttgttgctgg | |
| | actagtgatcactgtcattgtcattgttggagccattcttttcgtcccaggtgaatattcattaaagaat | |
| | gctactggccttggtttaattgtgacttctacagggatattaatattacttcactactatgtgtttagtac | |
| | agcgattggattaacctccttcgtcattgccatattggttattcaggtgatagcctatatcctcgctgtg | |
| | gttggactgagtctctgtattgcggcgtgtataccaatgcatggccctcttctgatttcaggtttgagta | |
| | tcttagctctagcacaattacttggactagtttatatgaaatttgtggcttccaatcagaagactataca | |
| | acctcctaggaataactga | |
| 545 | atgtggcccctggtcgccgccctgttgctgggctcggcatgctgcggatcagctcagctactgtttaat | Codon-optimized |
| | aaaacaaaatctgtagaattcacgttttgtaacgacactgtcgtgatcccatgctttgttactaatatg | nucleotide |
| | gaggcacaaaacaccactgaagtgtacgtgaagtggaaattcaaaggcagagacatttacaccttt | sequence encoding |
| | gacggcgccctcaacaagtccaccgtgcccactgactttagtagcgcaaaaattgaggtcagccaat | CD47 |
| | tactaaaaggagatgcctctttgaagatggacaagagcgatgctgtcagccacacagggaactaca | |
| | cttgtgaagtaacagagttaacccgcgaaggtgaaacgatcatcgagctgaagtatcgagtggtgtc | |
| | ctggttttctccgaacgagaatatccttatcgtaattttcccaattttcgctatcctcctgttctggggcc | |
| | agtttggtatcaagacactcaaatatcggtccggtgggatggatgagaagacaattgccctgcttgtt | |
| | gctggactcgtgatcaccgtcatcgtgattgttggggccatccttttcgtcccaggggagtacagcctg | |
| | aagaatgctacgggcctgggattaattgtgacctctacagggatactcatcctgcttcactactatgtg | |
| | ttcagtaccgcgattggactgacctccttcgtcattgccatattggtgattcaggtgatagcctacatcc | |
| | tcgccgtggttggcctgagtctctgtatcgcggcgtgcatacccatgcatggccctcttctgatttcag | |
| | ggttgagtatcctcgcactagcacagttgctgggactggtttatatgaaatttgtggcctccaaccag | |
| | aagactatacagcctcctaggaaggctgtagaggagcccctgaatgcattcaaggaatcaaaaggc | |
| | atgatgaatgatgaa | |

In some embodiments, the transgene comprises a nucleotide sequence encoding a CD4′7 polypeptide having at least 95% sequence identity (e.g., 95%, 96%, 97%, 98%, 99%, or more) to the amino acid sequence of SEQ ID NO:541. In some embodiments, the transgene comprises a nucleotide sequence encoding a CD47 polypeptide having the amino acid sequence of SEQ ID NO:541. In some embodiments, the transgene comprises a nucleotide sequence encoding a CD47 polypeptide having at least 95% sequence identity (e.g., 95%, 96%, 97%, 98%, 99%, or more) to the amino acid sequence of SEQ ID NO:542. In some embodiments, the transgene comprises a nucleotide sequence encoding a CD47 polypeptide having the nucleotide sequence of SEQ ID NO:543-545. In some embodiments, the nucleotide sequence is codon optimized for expression in a particular cell.

In some embodiments, a suitable gene editing system (e.g., CRISPR/Cas system or any of the gene editing systems described herein) is used to facilitate the insertion of a polynucleotide encoding CD47, into a genomic locus of the hypoimmunogenic cell. In some embodiments, the polynucleotide encoding CD47 is inserted into a safe harbor or target locus, such as but not limited to, an AAVS1, CCR5, CLYBL, ROSA26, SHS231, F3 (CD142), MICA, MICB, LRP1 (CD91), HMGB1, ABO, RHD, FUT1, or KDM5D gene locus. In some embodiments, the polynucleotide encoding CD47 is inserted into a B2M gene locus, a CIITA gene locus, a TRAC gene locus, or a TRB gene locus. In some embodiments, the polynucleotide encoding CD47 is operably linked to a promoter.

In some embodiments, the polynucleotide encoding CD47 is inserted into at least one allele of the T cell using viral transduction. In some embodiments, the polynucleotide encoding CD47 is inserted into at least one allele of the T cell using a lentivirus viral vector described herein. In some embodiments, the lentivirus viral vector is a pseudotyped, self-inactivating lentiviral vector that carries the polynucleotide encoding CD47. In some embodiments, the lentivirus viral vector is a self-inactivating lentiviral vector pseudotyped with a vesicular stomatitis VSV-G envelope, and which carries the polynucleotide encoding CD47.

In some embodiments, CD47 protein expression is detected using a Western blot of cell lysates probed with antibodies against the CD47 protein. In some embodiments, reverse transcriptase polymerase chain reactions (RT-PCR) are used to confirm the presence of the exogenous CD47 mRNA.

### (ii) CD24

In some embodiments, the present disclosure provides a transgene that encodes the tolerogenic factor (e.g., immunomodulatory polypeptide) CD24. CD24 which is also referred to as a heat stable antigen or small-cell lung cancer cluster 4 antigen is a glycosylated glycosylphosphatidylinositol-anchored surface protein (Pirruccello et al., J Immunol, 1986, 136, 3779-3784; Chen et al., Glycobiology, 2017, 57, 800-806). It binds to Siglec-10 on innate immune cells. Recently it has been shown that CD24 via Siglec-10 acts as an innate immune checkpoint (Barkal et al., Nature, 2019, 572, 392-396). In some embodiments, the exogenous polynucleotide is inserted into at least one allele of the cell using viral transduction with a lentiviral vector as described herein.

In some embodiments, the transgene disclosed herein comprises a nucleotide sequence encoding a CD24 polypeptide has at least 95% sequence identity (e.g., 95%, 96%, 97%, 98%, 99%, or more) to an amino acid sequence set forth in NCBI Ref. Nos. NP_001278666.1, NP_001278667.1, NP_001278668.1, and NP_037362.1. In some embodiments, the transgene disclosed herein comprises a nucleotide sequence encoding a CD24 polypeptide having an amino acid sequence set forth in NCBI Ref. Nos. NP_001278666.1, NP_001278667.1, NP_001278668.1, and NP_037362.1.

In some embodiments, the transgene comprises a nucleotide sequence having at least 85% sequence identity (e.g., 85%, 86%, 87%, 88%, 89%, 90%, 91%, 92%, 93%, 94%, 95%, 96%, 97%, 98%, 99%, or more) to the sequence set forth in NCBI Ref. Nos. NM_00129737.1, NM_00129738.1, NM_001291739.1, and NM_013230.3. In some embodiments, the transgene comprises a nucleotide sequence as set forth in NCBI Ref. Nos. NM_00129737.1, NM_00129738.1, NM_001291739.1, and NM_013230.3.

In some embodiments, a suitable gene editing system (e.g., CRISPR/Cas system or any of the gene editing systems described herein) is used to facilitate the insertion of a polynucleotide encoding CD24, into a genomic locus of the hypoimmunogenic cell. In some embodiments, the polynucleotide encoding CD24 is inserted into a safe harbor or target locus, such as but not limited to, an AAVS1, CCR5, CLYBL, ROSA26, SHS231, F3 (CD142), MICA, MICB, LRP1 (CD91), HMGB1, ABO, RHD, FUT1, or KDM5D gene locus. In some embodiments, the polynucleotide encoding CD24 is inserted into a B2M gene locus, a CIITA gene locus, a TRAC gene locus, or a TRB gene locus. In some embodiments, the polynucleotide encoding CD24 is operably linked to a promoter.

In some embodiments, CD24 protein expression is detected using a Western blot of cells lysates probed with antibodies against the CD24 protein. In some embodiments, reverse transcriptase polymerase chain reactions (RT-PCR) are used to confirm the presence of the exogenous CD24 mRNA.

In some embodiments, a suitable gene editing system (e.g., CRISPR/Cas system or any of the gene editing systems described herein) is used to facilitate the insertion of a polynucleotide encoding CD24, into a genomic locus of the hypoimmunogenic cell. In some embodiments, the polynucleotide encoding CD24 is inserted into a safe harbor or target locus, such as but not limited to, an AAVS1, CCR5, CLYBL, ROSA26, SHS231, F3 (also known as CD142), MICA, MICB, LRP1 (also known as CD91), HMGB1, ABO, RHD, FUT1, or KDM5D gene locus. In some embodiments, the polynucleotide encoding CD24 is inserted into a B2M gene locus, a CIITA gene locus, a TRAC gene locus, or a TRB gene locus. In some embodiments, the polynucleotide encoding CD24 is operably linked to a promoter.

(iii) DUX4

In some embodiments, the present disclosure provides a transgene that encodes DUX4. In some embodiments, increased expression of DUX4 suppresses, reduces or eliminates expression of one or more of the following MHC I molecules—HLA-A, HLA-B, and HLA-C. In some embodiments, the exogenous polynucleotide is inserted into at least one allele of the cell using viral transduction with a lentiviral vector described herein.

DUX4 is a transcription factor that is active in embryonic tissues and induced pluripotent stem cells, and is silent in normal, healthy somatic tissues (Feng et al., 2015, ELife4; De Iaco et al., 2017, Nat Genet, 49, 941-945; Hendrickson et al., 2017, Nat Genet, 49, 925-934; Snider et al., 2010, PLoS Genet, e1001181; Whiddon et al., 2017, Nat Genet). DUX4 expression acts to block IFN-gamma mediated induction of major histocompatibility complex (MHC) class I gene expression (e.g., expression of B2M, HLA-A, HLA-B, and HLA-C). DUX4 expression has been implicated in suppressed antigen presentation by MHC class I (Chew et al., Developmental Cell, 2019, 50, 1-14). DUX4 functions as a transcription factor in the cleavage-stage gene expression (transcriptional) program. Its target genes include, but are not limited to, coding genes, noncoding genes, and repetitive elements.

There are at least two isoforms of DUX4, with the longest isoform comprising the DUX4 C-terminal transcription activation domain. The isoforms are produced by alternative splicing. See, e.g., Geng et al., 2012, Dev Cell, 22, 38-51; Snider et al., 2010, PLoS Genet, e1001181. Active isoforms for DUX4 comprise its N-terminal DNA-binding domains and its C-terminal activation domain. See, e.g., Choi et al., 2016, Nucleic Acid Res, 44, 5161-5173.

It has been shown that reducing the number of CpG motifs of DUX4 decreases silencing of a DUX4 transgene (Jagannathan et al., Human Molecular Genetics, 2016, 25(20):4419-4431). The nucleic acid sequence provided in Jagannathan et al., supra represents a codon altered sequence of DUX4 comprising one or more base substitutions to reduce the total number of CpG sites while preserving the DUX4 protein sequence. The nucleic acid sequence is commercially available from Addgene, Catalog No. 99281.

In some embodiments, at least one or more transgenes encoding DUX4 is utilized to facilitate the exogenous expression of DUX4 by a cell, e.g., a stem cell, induced pluripotent stem cell, differentiated cell, hematopoietic stem cell, primary T cell or CAR-T cell.

In some embodiments, a suitable gene editing system (e.g., CRISPR/Cas system or any of the gene editing systems described herein) is used to facilitate the insertion of a polynucleotide encoding DUX4, into a genomic locus of the hypoimmunogenic cell. In some embodiments, the polynucleotide encoding DUX4 is inserted into a safe harbor or target locus, such as but not limited to, an AAVS1, CCR5, CLYBL, ROSA26, SHS231, F3 (CD142), MICA, MICB, LRP1 (CD91), HMGB1, ABO, RHD, FUT1, or KDM5D gene locus. In some embodiments, the polynucleotide encoding DUX4 is inserted into a B2M gene locus, a CIITA gene locus, a TRAC gene locus, or a TRB gene locus. In some embodiments, the polynucleotide encoding DUX4 is operably linked to a promoter.

In some embodiments, the polynucleotide encoding DUX4 is inserted into at least one allele of the T cell using viral transduction. In some embodiments, the polynucleotide encoding DUX4 is inserted into at least one allele of the T cell using a lentivirus based viral vector. In some embodiments, the lentivirus based viral vector is a pseudotyped, self-inactivating lentiviral vector that carries the polynucleotide encoding DUX4. In some embodiments, the lentivirus based viral vector is a self-inactivating lentiviral vector pseudotyped with a vesicular stomatitis VSV-G envelope, and which carries the polynucleotide encoding DUX4.

In some embodiments, the polynucleotide sequence encoding DUX4 comprises a polynucleotide sequence comprising a codon altered nucleotide sequence of DUX4 comprising one or more base substitutions to reduce the total number of CpG sites while preserving the DUX4 protein sequence. In some embodiments, the polynucleotide sequence encoding DUX4 comprising one or more base substitutions to reduce the total number of CpG sites has at least 85% (e.g., 85%, 86%, 87%, 88%, 89%, 90%, 91%, 92%, 93%, 94%, 95%, 96%, 97%, 98%, 99% or 100%) sequence identity to SEQ ID NO:1 of PCT/US2020/44635, filed Jul. 31, 2020. In some embodiments, the polynucleotide sequence encoding DUX4 is SEQ ID NO:1 of PCT/US2020/44635.

In some embodiments, the polynucleotide sequence encoding DUX4 is a nucleotide sequence encoding a polypeptide sequence having at least 95% (e.g., 95%, 96%, 97%, 98%, 99% or 100%) sequence identity to a sequence selected from a group including SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, and SEQ ID NO:29, as provided in PCT/US2020/44635. In some embodiments, the polynucleotide sequence encoding DUX4 is a nucleotide sequence encoding a polypeptide sequence is selected from a group including SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, and SEQ ID NO:29. Amino acid sequences set forth as SEQ ID NOS:2-29 are shown in FIG. 1A-1G of PCT/US2020/44635.

In some instances, the DUX4 polypeptide comprises an amino acid sequence having at least 95% sequence identity to SEQ ID NO:28 provided in PCT/US2020/44635 or an amino acid sequence of SEQ ID NO:28 provided in PCT/US2020/44635. In some instances, the DUX4 polypeptide comprises an amino acid sequence having at least 95% sequence identity to SEQ ID NO:29 provided in PCT/US2020/44635 or an amino acid sequence of SEQ ID NO:29 provided in PCT/US2020/44635.

An increase of DUX4 expression is assayed using known techniques, such as Western blots, ELISA assays, FACS assays, immunoassays, and the like.

### (iv) Additional Tolerogenic Factors

In some embodiments, one or more tolerogenic factors is inserted or reinserted into genome-edited cells to create immune-privileged universal donor cells, such as universal donor stem cells, universal donor T cells, or universal donor cells. In some embodiments, the hypoimmunogenic cells disclosed herein have been further modified to express one or more tolerogenic factors. Exemplary tolerogenic factors include, without limitation, one or more of CD47, DUX4, CD24, CD27, CD35, CD46, CD55, CD59, CD200, HLA-C, HLA-E, HLA-E heavy chain, HLA-G, PD-L1, IDO1, CTLA4-Ig, C1-Inhibitor, IL-10, IL-35, IL-39 FasL, CCL21, CCL22, Mfge8, CD16, CD52, H2-M3, CD16 Fc receptor, IL15-RF, H2-M3(HLA-G), B2M-HLA-E, A20/TNFAIP3, CR1, HLA-F, MANF, Serpinb9, and a SIRPα-binding polypeptide (e.g. a SIRPα engager). In some embodiments, the tolerogenic factors are selected from the group consisting of CD200, HLA-G, HLA-E, HLA-C, HLA-E heavy chain, PD-L1, IDO1, CTLA4-Ig, IL-10, IL-35, FasL, Serpinb9, CCL21, CCL22, and Mfge8. In some embodiments, the tolerogenic factors are selected from the group consisting of DUX4, HLA-C, HLA-E, HLA-F, HLA-G, PD-L1, CTLA-4-Ig, C1-inhibitor, and IL-35. In some embodiments, the tolerogenic factors are selected from the group consisting of HLA-C, HLA-E, HLA-F, HLA-G, PD-L1, CTLA-4-Ig, C1-inhibitor, and IL-35. In some embodiments, the tolerogenic factors are selected from a group including CD47, DUX4, CD24, CD27, CD35, CD46, CD55, CD59, CD200, HLA-C, HLA-E, HLA-E heavy chain, HLA-G, PD-L1, IDO1, CTLA4-Ig, C1-Inhibitor, IL-10, IL-35, IL-39 FasL, CCL21, CCL22, Mfge8, CD16, CD52, H2-M3, CD16 Fc receptor, IL15-RF, H2-M3(HLA-G), B2M-HLA-E, A20/TNFAIP3, CR1, HLA-F, and MANF, and Serpinb9.

A SIRPα-binding polypeptide (e.g. a SIRPα engager) is an engager molecule on a cell surface that engages with a Signal Regulatory Protein Alpha (SIRPα) protein on an immune cell, wherein the engagement prevents the engager cell from being killed by said immune cell, wherein the engager molecule lacks a functional CD47 intracellular domain. The SIRPα engager can be a fusion protein. The fusion protein may include a CD47 extracellular domain and a transmembrane domain or membrane anchor, wherein the transmembrane domain or membrane anchor are not found in wild-type CD47. The SIRPα engager can be a fusion protein comprising an antibody or scFv that targets and activates SIPRα signaling on an immune cell. Examples of SIRPα engagers are disclosed in US App. Pub. No. US20240043803, herein incorporated by reference.

In some embodiments, the polynucleotide encoding the one or more tolerogenic factors is inserted into at least one allele of the T cell using viral transduction. In some embodiments, the polynucleotide encoding the one or more tolerogenic factors is inserted into at least one allele of the T cell using a lentivirus viral vector. In some embodiments, the lentivirus viral vector is a pseudotyped, self-inactivating lentiviral vector that carries the polynucleotide encoding the one or more tolerogenic factors. In some embodiments, the lentivirus viral vector is a self-inactivating lentiviral vector pseudotyped with a vesicular stomatitis VSV-G envelope, and which carries the polynucleotide encoding the one or more tolerogenic factors.

Useful genomic, polynucleotide and polypeptide information about human CD27 (which is also known as CD27L receptor, Tumor Necrosis Factor Receptor Superfamily Member 7, TNFSF7, T Cell Activation Antigen S152, Tp55, and T14) are provided in, for example, the GeneCard Identifier GC12P008144, HGNC No. 11922, NCBI Gene ID 939, Uniprot No. P26842, and NCBI RefSeq Nos. NM_001242.4 and NP_001233.1.

Useful genomic, polynucleotide and polypeptide information about human CD46 are provided in, for example, the GeneCard Identifier GC01P207752, HGNC No. 6953, NCBI Gene ID 4179, Uniprot No. P15529, and NCBI RefSeq Nos. NM_002389.4, NM_153826.3, NM_172350.2, NM_172351.2, NM_172352.2 NP_758860.1, NM_172353.2, NM_172359.2, NM_172361.2, NP_002380.3, NP_722548.1, NP_758860.1, NP_758861.1, NP_758862.1, NP_758863.1, NP_758869.1, and NP_758871.1.

Useful genomic, polynucleotide and polypeptide information about human CD55 (also known as complement decay-accelerating factor) are provided in, for example, the GeneCard Identifier GC01P207321, HGNC No. 2665, NCBI Gene ID 1604, Uniprot No. P08174, and NCBI RefSeq Nos. NM_000574.4, NM_001114752.2, NM_001300903.1, NM_001300904.1, NP_000565.1, NP_001108224.1, NP_001287832.1, and NP_001287833.1.

Useful genomic, polynucleotide and polypeptide information about human CD59 are provided in, for example, the GeneCard Identifier GC11M033704, HGNC No. 1689, NCBI Gene ID 966, Uniprot No. P13987, and NCBI RefSeq Nos. NP_000602.1, NM_000611.5, NP_001120695.1, NM_001127223.1, NP_001120697.1, NM_001127225.1, NP_001120698.1, NM_001127226.1, NP_001120699.1, NM_001127227.1, NP_976074.1, NM_203329.2, NP_976075.1, NM_203330.2, NP_976076.1, and NM_203331.2.

Useful genomic, polynucleotide and polypeptide information about human CD200 are provided in, for example, the GeneCard Identifier GC03P112332, HGNC No. 7203, NCBI Gene ID 4345, Uniprot No. P41217, and NCBI RefSeq Nos. NP_001004196.2, NM_001004196.3, NP_001305757.1, NM_001318828.1, NP_005935.4, NM_005944.6, XP_005247539.1, and XM_005247482.2.

Useful genomic, polynucleotide and polypeptide information about human HLA-C are provided in, for example, the GeneCard Identifier GC06M031272, HGNC No. 4933, NCBI Gene ID 3107, Uniprot No. P10321, and NCBI RefSeq Nos. NP_002108.4 and NM_002117.5.

Useful genomic, polynucleotide and polypeptide information about human HLA-E are provided in, for example, the GeneCard Identifier GC06P047281, HGNC No. 4962, NCBI Gene ID 3133, Uniprot No. P13747, and NCBI RefSeq Nos. NP_005507.3 and NM_005516.5.

Useful genomic, polynucleotide and polypeptide information about human HLA-G are provided in, for example, the GeneCard Identifier GC06P047256, HGNC No. 4964, NCBI Gene ID 3135, Uniprot No. P17693, and NCBI RefSeq Nos. NP_002118.1 and NM_002127.5.

Useful genomic, polynucleotide and polypeptide information about human PD-L1 or CD274 are provided in, for example, the GeneCard Identifier GC09P005450, HGNC No. 17635, NCBI Gene ID 29126, Uniprot No. Q9NZQ7, and NCBI RefSeq Nos. NP_001254635.1, NM 001267706.1. NP_054862.1, and NM_014143.3.

Useful genomic, polynucleotide and polypeptide information about human IDO1 are provided in, for example, the GeneCard Identifier GC08P039891, HGNC No. 6059, NCBI Gene ID 3620, Uniprot No. P14902, and NCBI RefSeq Nos. NP_002155.1 and NM_002164.5.

Useful genomic, polynucleotide and polypeptide information about human IL-10 are provided in, for example, the GeneCard Identifier GC01M206767, HGNC No. 5962, NCBI Gene ID 3586, Uniprot No. P22301, and NCBI RefSeq Nos. NP_000563.1 and NM_000572.2.

Useful genomic, polynucleotide and polypeptide information about human Fas ligand (which is known as FasL, FASLG, CD178, TNFSF6, and the like) are provided in, for example, the GeneCard Identifier GC01P172628, HGNC No. 11936, NCBI Gene ID 356, Uniprot No. P48023, and NCBI RefSeq Nos. NP_000630.1, NM_000639.2, NP_001289675.1, and NM_001302746.1.

Useful genomic, polynucleotide and polypeptide information about human CCL21 are provided in, for example, the GeneCard Identifier GC09M034709, HGNC No. 10620, NCBI Gene ID 6366, Uniprot No. 000585, and NCBI RefSeq Nos. NP_002980.1 and NM_002989.3.

Useful genomic, polynucleotide and polypeptide information about human CCL22 are provided in, for example, the GeneCard Identifier GC16P057359, HGNC No. 10621, NCBI Gene ID 6367, Uniprot No. 000626, and NCBI RefSeq Nos. NP_002981.2, NM_002990.4, XP_016879020.1, and XM_017023531.1.

Useful genomic, polynucleotide and polypeptide information about human Mfge8 are provided in, for example, the GeneCard Identifier GC15M088898, HGNC No. 7036, NCBI Gene ID 4240, Uniprot No. Q08431, and NCBI RefSeq Nos. NP_001108086.1, NM_001114614.2, NP_001297248.1, NM_001310319.1, NP_001297249.1, NM_001310320.1, NP_001297250.1, NM_001310321.1, NP_005919.2, and NM_005928.3.

Useful genomic, polynucleotide and polypeptide information about human SerpinB9 are provided in, for example, the GeneCard Identifier GC06M002887, HGNC No. 8955, NCBI Gene ID 5272, Uniprot No. P50453, and NCBI RefSeq Nos. NP_004146.1, NM_004155.5, XP_005249241.1, and XM_005249184.4.

In some embodiments, the lentiviral vector comprising two or more genomes is used to transduced cells (e.g., stem cell, induced pluripotent stem cell, differentiated cell, hematopoietic stem cell, primary T cell or CAR-T cell) that possess genetic modifications that inactivate the B2M and CIITA genes. The lentiviral vector comprising two or more genomes introduces two or more transgenes to the cell. The transduced cells express a plurality of exogenous polypeptides selected from the group including CD47 and DUX4, CD47 and CD24, CD47 and CD27, CD47 and CD35, CD47 and CD46, CD47 and CD55, CD47 and CD59, CD47 and CD200, CD47 and HLA-C, CD47 and HLA-E, CD47 and HLA-E heavy chain, CD47 and HLA-G, CD47 and PD-L1, CD47 and IDO1, CD47 and CTLA4-Ig, CD47 and C1-Inhibitor, CD47 and IL-10, CD47 and IL-35, CD47 and IL-39, CD47 and FasL, CD47 and CCL21, CD47 and CCL22, CD47 and Mfge8, CD47 and CD16, CD47 and CD52, CD47 and CD16 Fe receptor, CD47 and IL15-RF, CD47 and H2-M3(HLA-G), CD47 and B2M-HLA-E, CD47 and A20/TNFAIP3, CD47 and CR1, CD47 and HLA-F, CD47 and MANF, and CD47 and Serpinb9, and any combination thereof. In some instances, such cells also possess a genetic modification that inactivates the CD142 gene.

### 3. Multicistronic Constructs

In some embodiments, the first genome and/or the second genome of the lentiviral vector comprises a bicistronic or multicistronic construct. Multicistronic constructs have two or more expression cassettes for co-expression of two or more proteins of interest in a target cell. In some embodiments, the multicistronic construct comprises two expression cassettes, i.e., is bicistronic. In some embodiments, the multicistronic construct comprises three expression cassettes, i.e., is tricistronic. In some embodiments, the multicistronic construct comprises four expression cassettes, i.e., is quadcistronic. In some embodiments, the multicistronic construct comprises more than four expression cassettes. In any of these embodiments, each of the expression cassettes comprises a nucleotide sequence encoding a protein of interest (e.g., a tolerogenic factor, a suicide switch, a regulatory factor, an antibody or antigen binding fragment thereof, or a CAR). In some embodiments, the two or more genes being expressed are under the control of a single promoter and are separated from one another by one or more cleavage sites to achieve co-expression of the proteins of interest from one transcript. In other embodiments, the two or more genes are under the control of separate promoters.

In some embodiments, the two or more expression cassettes of the multicistronic construct are separated by one or more cleavage sites. As the name suggests, a multicistronic construct allows simultaneous expression of two or more separate proteins from one mRNA transcript in a host cell. Cleavage sites are used in the design of a multicistronic construct to achieve such co-expression of multiple genes.

In some embodiments, the one or more cleavage sites comprise one or more self-cleaving sites. In some embodiments, the self-cleaving site comprises a 2A site. 2A peptides are a class of 18-22 amino acid-long peptides first discovered in picornaviruses and can induce ribosomal skipping during translation of a protein, thus producing equal amounts of multiple genes from the same mRNA transcript. 2A peptides function to “cleave” an mRNA transcript by making the ribosome skip the synthesis of a peptide bond at the C-terminus, between the glycine (G) and proline (P) residues, leading to separation between the end of the 2A sequence and the next peptide downstream. There are four 2A peptides commonly employed in molecular biology, T2A, P2A, E2A, and F2A, the sequences of which are summarized in Table 23. A glycine-serine-glycine (GSG) linker is optionally added to the N-terminal of a 2A peptide to increase cleavage efficiency. The use of “(” around a sequence in the present disclosure means that the enclosed sequence is optional.

In some embodiments, the one or more cleavage sites additionally comprise one or more protease sites. The one or more protease sites can either precede or follow the self-cleavage sites (e.g., 2A sites) in the 5′ to 3′ order. The protease site is cleaved by a protease after translation of the full transcript or after translation of each expression cassette such that the first expression product is released prior to translation of the next expression cassette. In these embodiments, having a protease site in addition to the 2A site, especially preceding the 2A site in the 5′ to 3′ order, may reduce the number of extra amino acid residues attached to the expressed proteins of interest. In some embodiments, the protease site comprises a furin site, also known as a Paired basic Amino acid Cleaving Enzyme (PACE) site. There are at least three furin cleavage sequences, FC1, FC2, and FC3, the amino acid sequences of which are summarized in Table 24. In some embodiments, one or more optional glycine-serine-glycine (GSG) sequences are included for cleavage efficiency.

In some embodiments, the one or more cleavage sites comprise one or more self-cleaving sites, one or more protease sites, and/or any combination thereof. For example, the cleavage site includes a 2A site alone. For another example, the cleavage site includes a FC2 or FC3 site, followed by a 2A site. In these embodiments, the one or more self-cleaving sites are the same or different. In some embodiments, the one or more protease sites are the same or different.

In some embodiments, the polycistronic construct are in the form of a vector. In some embodiments, any type of vector suitable for introduction of nucleotide sequences into a host cell is used, including, for example, plasmids, adenoviral vectors, adenoviral-associated vectors, retroviral vectors, lentiviral vectors, phages, and homology-directed repair (HDR)-based donor vectors.

TABLE 23
Sequences of 2A peptides

| SEQ | | |
|---|---|---|
| ID NO: | Amino Acid Sequence | 2A Peptide |
| 546 | (GSG) EGRGSLLTCGDVEENPGP | T2A |
| 547 | (GSG) ATNFSLLKQAGDVEENPGP | P2A |
| 548 | (GSG) QCTNYALLKLAGDVESNPGP | E2A |
| 549 | (GSG) VKQTLNFDLLKLAGDVESNPGP | F2A |

TABLE 24
Sequences of furin sites

| SEQ | | |
|---|---|---|
| ID NO: | Amino Acid Sequence | Furin site |
| 552 | RRRR (GSG) | FC1 |
| 553 | RKRR (GSG) | FC2 |
| 554 | RKRR (GSG) TPDPW (GSG) | FC3 |

### 4. Gene-Editing Agents

In some embodiments, described herein is an engineered lentiviral vector including a first genome and a second genome, wherein the first genome includes a first transgene and the second genome includes a second transgene, and wherein the first and the second transgene are different. In some embodiments, the first transgene encodes a payload agent that is associated with a gene editing technology. In some embodiments, the second transgene encodes a payload agent that is associated with a gene editing technology. Any of a variety of agents associated with gene editing technologies can be included as the payload agent, such as for delivery of gene editing machinery to a cell. In some embodiments, the gene editing technology can include systems involving nucleases, integrases, transposases, recombinases. In some embodiments, the gene editing technologies can be used for knock-out or knock-down of genes. In some embodiments, the gene-editing technologies can be used for knock-in or integration of DNA into a region of the genome. In some embodiments, the payload agent mediates double-strand breaks (DSB), including in connection with non-homologous end-joining (NHEJ) or homology-directed repair (HDR). In some embodiments, the payload agent does not mediate DSB. In some embodiments, the payload agent can be used for DNA-based editing or prime-editing. In some embodiments, the payload agent can be used for Programmable Addition via Site-specific Targeting Elements (PASTE).

In some embodiments, a transgene encodes a payload agent that is a nuclease. In some embodiments, a nuclease is a CRISPR-associated protein (Cas) nuclease, a transcription activator-like effector nuclease (TALEN), a meganuclease, or a zinc-finger nuclease (ZFN). Viral vectors including transgenes encoding a nuclease can be useful for applications in which a viral vector is used to introduce genetic modifications into a cell. In such cases, a viral vector can enter into a cell and express the nuclease. In some embodiments, a viral vector may encode an RNA (e.g., a gRNA) and a polypeptide (e.g., a Cas nuclease) to impart targeted genetic modifications.

In some embodiments, a first transgene encodes a gRNA and a second transgene encodes a Cas nuclease.

In some embodiments, the payload agent is a nuclease for use in gene editing methods, such as a sequence-specific nuclease. In some embodiments, the nuclease is a zinc-finger nucleases (ZFNs), transcription-activator like effector nucleases (TALENs), or a CRISPR-associated protein-nuclease (Cas). In some embodiments, the Cas protein is selected from the group consisting of Cas3, Cas9, Cas10, Cas12, and Cas13. In some embodiments, the Cas is Cas9 from Streptococcus pyogenes. In some embodiments, the Cas is Cas9 from Streptococcus pyogenes (SpCas). In some embodiments, the Cas9 is from Staphylococcus aureus (SaCas9). In some embodiments, the Cas9 is from Neisseria meningitidis (NmeCas9). In some embodiments, the Cas9 is from Campylobacter jejuni (CjCas9). In some embodiments, the Cas9 is from Streptococcus thermophilis (StCas9). In some embodiments, the Cas is a Cas12a (also known as Cpf1) from a Prevotella or Francisella bacteria, or the Cas is a Cas12b from a Bacillus, optionally Bacillus hisashii. In some embodiments, the Cas is a Cas12a (also known as cpf1) from a Prevotella, Francisella novicida, Acidaminococcus sp., Lachnospiraceae bacterium, or Francisella bacteria. In some embodiments, the nuclease is MAD7 or CasX. In some of any embodiments, the Cas is a Cas3, Cas13, CasMini, or any other Cas protein known in the art. See for example, Wang et al., Biosensors and Bioelectronics (165) 1: 2020, and Wu et al. Nature Reviews Chemistry (4) 441: 2020). The Cas9 nuclease can, in some embodiments, be a Cas9 or functional fragment thereof from any bacterial species. See, e.g., Makarova et al. Nature Reviews, Microbiology, 9: 467-477 (2011), including supplemental information, hereby incorporated by reference in its entirety.

In some embodiments, the Cas is wild-type Cas9, which can site-specifically cleave double-stranded DNA, resulting in the activation of the double-strand break (DSB) repair machinery. DSBs can be repaired by the cellular Non-Homologous End Joining (NHEJ) pathway (Overballe-Petersen et al., 2013, Proc Natl Acad Sci USA, Vol. 110: 19860-19865), resulting in insertions and/or deletions (indels) which disrupt the targeted locus. Alternatively, if a donor template with homology to the targeted locus is supplied, the DSB may be repaired by the homology-directed repair (HDR) pathway allowing for precise replacement mutations to be made (Overballe-Petersen et al., 2013, Proc Natl Acad Sci USA, Vol. 110: 19860-19865; Gong et al., 2005, Nat. Struct Mol Biol, Vol. 12: 304-312). In some embodiments, the Cas is mutant form, known as Cas9 D10A, with only nickase activity. This means that Cas9D10A cleaves only one DNA strand, and does not activate NHEJ. Instead, when provided with a homologous repair template, DNA repairs are conducted via the high-fidelity HDR pathway only, resulting in reduced indel mutations (Cong et al., 2013, Science, Vol. 339: 819-823; Jinek et al., 2012, Science, Vol. 337: 816-821; Qi et al., 2013 Cell, Vol. 152: 1173-1183). Cas9D10A is even more appealing in terms of target specificity when loci are targeted by paired Cas9 complexes designed to generate adjacent DNA nicks (Ran et al., 2013, Cell, Vol. 154: 1380-1389). In some embodiments, the Cas is a nuclease-deficient Cas9 (Qi et al., 2013 Cell, Vol. 152: 1173-1183). For instance, mutations H840A in the HNH domain and D10A in the RuvC domain inactivate cleavage activity, but do not prevent DNA binding. Therefore, this variant can be used to target in a sequence-specific manner any region of the genome without cleavage. Instead, by fusing with various effector domains, dCas9 can be used either as a gene silencing or activation tools. Furthermore, it can be used as a visualization tool by coupling the guide RNA or the Cas9 protein to a fluorophore or a fluorescent protein.

In some embodiments, the Cas protein comprises one or more mutations such that the Cas protein is converted into a nickase that lacks the ability to cleave both strands of a double stranded DNA molecule. In some embodiments, the Cas protein comprises one or more mutations such that the Cas protein is converted into a nickase that is able to cleave only one strand of a double stranded DNA molecule (e.g., a SSB). For example, Cas9, which is normally capable of inducing a double strand break, can be converted into a Cas9 nickase, which is capable of inducing a single strand break, by mutating one of two Cas9 catalytic domains: the RuvC domain, which comprises the RuvC I, RuvC II, and RuvC III motifs, or the NHN domain. In some embodiments, the Cas protein comprises one or more mutations in the RuvC catalytic domain or the HNH catalytic domain. In some embodiments, the genome-modifying protein is a recombinant nuclease that has been modified to have nickase activity. In some embodiments, the recombinant nuclease cleaves the strand to which the guide RNA, e.g., sgRNA, hybridizes, but does not cleave the strand that is complementary to the strand to which the guide RNA, e.g., sgRNA, hybridizes. In some embodiments, the recombinant nuclease does not cleave the strand to which the guide RNA, e.g., sgRNA, hybridizes, but does cleave the strand that is complementary to the strand to which the guide RNA, e.g., sgRNA, hybridizes.

In some embodiments, the Cas protein is selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, and Mad7. In some embodiments, the Cas protein is Cas9. In some embodiments, the Cas9 is from a bacteria selected from the group consisting of Streptococcus pyogenes, Staphylococcus aureus, Neisseria meningitides, Campylobacter jejuni, and Streptococcus thermophilis. In some embodiments, the Cas9 is from Streptococcus pyogenes. In some embodiments, the Cas9 is from Streptococcus pyogenes and comprises one or more mutations in the RuvC I, RuvC II, or RuvC III motifs. In some embodiments, the Cas9 is from Streptococcus pyogenes and comprises a D10A mutation in the RuvC I motif. In some embodiments, the Cas9 is from Streptococcus pyogenes and comprises one or more mutations in the HNH catalytic domain. In some embodiments, the Cas9 is from Streptococcus pyogenes and comprises one or more mutations in the HNH catalytic domain selected from the group consisting of H840A, H854A, and H863A. In some embodiments, the Cas9 is from Streptococcus pyogenes and comprises a H840A mutation in the HNH catalytic domain. In some embodiments, the Cas9 is from Streptococcus pyogenes and comprises a mutation selected from the group consisting of D10A, H840A, H854A, and H863A.

In some embodiments, the one or more agent(s) (e.g., the heterologous protein) capable of inducing a DSB comprise Cas9 or a functional fragment thereof, and a first guide RNA, e.g., a first sgRNA, and a second guide RNA, e.g., a second sgRNA. The guide RNA, e.g., the first guide RNA or the second guide RNA, in some embodiments, binds to the recombinant nuclease and targets the recombinant nuclease to a specific location within the target gene such as at a location within the sense strand or the antisense strand of the target gene that is or includes the cleavage site. In some embodiments, the recombinant nuclease is a Cas protein from any bacterial species, or is a functional fragment thereof. In some embodiments, the Cas protein is Cas9 nuclease. Cas9 can, in some embodiments, be a Cas9 or functional fragment thereof from any bacterial species. See, e.g., Makarova et al. Nature Reviews, Microbiology, 9: 467-477 (2011), including supplemental information, hereby incorporated by reference in its entirety. In some embodiments, the Cas9 is from Streptococcus pyogenes (SpCas9). In some embodiments, the Cas9 is from Staphylococcus aureus (SaCas9). In some embodiments, the Cas9 is from Neisseria meningitidis (NmeCas9). In some embodiments, the Cas9 is from Campylobacter jejuni (CjCas9). In some embodiments, the Cas9 is from Streptococcus thermophilis (StCas9).

In some embodiments, the Cas9 is from Streptococcus pyogenes (SpCas9) and comprises one or more mutations in the RuvC catalytic domain or the HNH catalytic domain. In some embodiments, the one or more mutations in the RuvC catalytic domain or the HNH catalytic domain inactivates the catalytic activity of the domain. In some embodiments, the recombinant nuclease has RuvC activity but does not have HNH activity. In some embodiments, the recombinant nuclease does not have RuvC activity but does have HNH activity. In some embodiments, the Cas9 is from Streptococcus pyogenes (SpCas9) and comprises one or more mutations selected from the group consisting of D10A, H840A, H854A, and H863A. In some embodiments, the Cas9 is from Streptococcus pyogenes (SpCas9) and comprises one or more mutations in the RuvC I, RuvC II, or RuvC III motifs. In some embodiments, the Cas9 is from Streptococcus pyogenes (SpCas9) and comprises a mutation in the RuvC I motif. In some embodiments, the Cas9 is from Streptococcus pyogenes (SpCas9) and comprises a D10A mutation in the RuvC I motif. In some embodiments, the Cas9 is from Streptococcus pyogenes (SpCas9) and comprises one or more mutations in the HNH catalytic domain. In some embodiments, the one or more mutations in the HNH catalytic domain is selected from the group consisting of H840A, H854A, and H863A. In some embodiments, the Cas9 is from Streptococcus pyogenes (SpCas9) and comprises a H840A mutation in the HNH catalytic domain. In some embodiments, the Cas9 is from Streptococcus pyogenes (SpCas9) and comprises a H840A mutation. In some embodiments, the Cas9 is from Streptococcus pyogenes (SpCas9) and comprises a D10A mutation. In some embodiments, the Cas9 is from Streptococcus pyogenes (SpCas9) and comprises one or more mutations selected from the group consisting of N497A, R661A, Q695A, and Q926A. In some embodiments, the Cas9 is from Streptococcus pyogenes (SpCas9) and comprises one or more mutations selected from the group consisting of R780A, K810A, K855A, H982A, K1003A, R1060A, and K848A. In some embodiments, the Cas9 is from Streptococcus pyogenes (SpCas9) and comprises one or more mutations selected from the group consisting of N692A, M694A, Q695A, and H698A. In some embodiments, the Cas9 is from Streptococcus pyogenes (SpCas9) and comprises one or more mutations selected from the group consisting of M495V, Y515N, K526E, and R661Q. In some embodiments, the Cas9 is from Streptococcus pyogenes (SpCas9) and comprises one or more mutations selected from the group consisting of F539S, M7631, and K890N. In some embodiments, the Cas9 is from Streptococcus pyogenes (SpCas9) and comprises one or more mutations selected from the group consisting of E480K, E543D, E1219V, A262T, 54091, M6941, E108G, S217A.

In some embodiments, the Cas9 is from Streptococcus pyogenes (SaCas9). In some embodiments, the SaCas9 is wild type SaCas9. In some embodiments, the SaCas9 comprises one or more mutations in REC3 domain. In some embodiments, the SaCas9 comprises one or more mutations in REC1 domain. In some embodiments, the SaCas9 comprises one or more mutations selected from the group consisting of N260D, N260Q, N260E, Q414A, Q414L. In some embodiments, the SaCas9 comprises one or more mutations in the recognition lobe. In some embodiments, the SaCas9 comprises one or more mutations selected from the group consisting of R245A, N413A, N419A. In some embodiments, the SaCas9 comprises one or more mutations in the RuvC-III domain. In some embodiments, the SaCas9 comprises a R654A mutation.

In some embodiments, the Cas protein is Cas12. In some embodiments, the Cas protein is Cas12a (i.e. cpf1). In some embodiments, the Cas12a is from the group consisting of Francisella novicida U112 (FnCas12a), Acidaminococcus sp. BV3L6 (AsCas12a), Moraxella bovoculi AAX11_00205 (Mb3Cas12a), Lachnospiraceae bacterium ND2006 (LbCas12a), Thiomicrospira sp. Xs5 (TsCas12a), Moraxella bovoculi AAX08_00205 (Mb2Cas12a), and Butyrivibrio sp. NC3005 (BsCas12a). In some embodiments, the Cas12a recognizes a T-rich 5′ protospacer adjacent motif (PAM). In some embodiments, the Cas12a processes its own crRNA without requiring a transactivating crRNA (tracrRNA). In some embodiments, the Cas12a processes both RNase and DNase activity. In some embodiments, the Cas12a is a split Cas12a platform, consisting of N-terminal and C-terminal fragments of Cas12a. In some embodiments, the split Cas12a platform is from Lachnospiraceae bacterium.

In some embodiments, the one or more agent(s) (e.g., one or more exogenous agent and/or heterologous protein) comprise, or are used in combination with, a guide RNA, e.g., single guide RNA (sgRNA), for inducing a DSB at the cleavage site. In some embodiments, the one or more agent(s) comprise, or are used in combination with, more than one guide RNA, e.g., a first sgRNA and a second sgRNA, for inducing a DSB at the cleavage site through a SSB on each strand. In some embodiments, the one or more agent(s) (e.g., the heterologous protein) can be used in combination with a donor template, e.g., a single-stranded DNA oligonucleotide (ssODN), for HDR-mediated integration of the donor template into the target gene, such as at the targeting sequence. In some embodiments, the one or more agent(s) (e.g., one or more exogenous agent and/or heterologous protein) can be used in combination with a donor template, e.g., an ssODN, and a guide RNA, e.g., a sgRNA, for HDR-mediated integration of the donor template into the target gene, such as at the targeting sequence. In some embodiments, the one or more agent(s) (e.g., one or more exogenous agent and/or heterologous protein) can be used in combination with a donor template, e.g., an ssODN, and a first guide RNA, e.g., a first sgRNA, and a second guide RNA, e.g., a second sgRNA, for HDR-mediated integration of the donor template into the target gene, such as at the targeting sequence.

In some embodiments, the genome-modifying protein, e.g., Cas9, is targeted to the cleavage site by interacting with a guide RNA, e.g., a first guide RNA, such as a first sgRNA, or a second guide RNA, such as a second sgRNA, that hybridizes to a DNA sequence on the sense strand or the antisense strand that immediately precedes a Protospacer Adjacent Motif (PAM) sequence.

In some embodiments, the genome-modifying agent, e.g., Cas9, is targeted to the cleavage site on the sense strand by interacting with a first guide RNA, e.g., first sgRNA, that hybridizes to a sequence on the sense strand that immediately precedes a PAM sequence. In some embodiments, the genome-modifying agent, e.g., Cas9, is targeted to the cleavage site on the antisense strand by interacting with a second guide RNA, e.g., second sgRNA, that hybridizes to a sequence on the antisense strand that immediately precedes a PAM sequence.

In some embodiments, the first guide RNA, e.g., first sgRNA, that is specific to the sense strand of a target gene of interest is used to target the recombinant nuclease, e.g., Cas9, to induce a SSB at a cleavage site within the sense strand of the target gene. In some embodiments, the first guide RNA, e.g., first sgRNA, that is specific to the antisense strand of a target gene of interest is used to target the recombinant nuclease, e.g., Cas9, to induce a SSB at a cleavage site within the antisense strand of the target gene.

In some embodiments, the second guide RNA, e.g., second sgRNA, that is specific to the sense strand of a target gene of interest used to target the recombinant nuclease, e.g., Cas9, to induce a SSB at a cleavage site within the sense strand of the target gene. In some embodiments, the second guide RNA, e.g., second sgRNA, that is specific to the antisense strand of a target gene of interest is used to target the recombinant nuclease, e.g., Cas9, to induce a SSB at a cleavage site within the antisense strand of the target gene.

In some embodiments, the first guide RNA, e.g., first sgRNA, that is specific to the sense strand of a target gene of interest is used to target the recombinant nuclease, e.g., Cas9, to induce a SSB at a cleavage site within the sense strand of the target gene; and the second guide RNA, e.g., second sgRNA, that is specific to the antisense strand of a target gene of interest is used to target the recombinant nuclease, e.g., Cas9, to induce a SSB at a cleavage site within the antisense strand of the target gene.

In some embodiments, the first guide RNA, e.g., first sgRNA, that is specific to the antisense strand of a target gene of interest is used to target the recombinant nuclease, e.g., Cas9, to induce a SSB at a cleavage site within the antisense strand of the target gene; and the second guide RNA, e.g., second sgRNA, that is specific to the sense strand of a target gene of interest is used to target the recombinant nuclease, e.g., Cas9, to induce a SSB at a cleavage site within the sense strand of the target gene. In general, a guide RNA, e.g., a first guide RNA, such as a first sgRNA, or a second guide RNA, such as a second sgRNA, is any nucleotide sequence comprising a sequence, e.g., a crRNA sequence, that has sufficient complementarity with a target gene sequence to hybridize with the target gene sequence at the cleavage site and direct sequence-specific binding of the recombinant nuclease to a portion of the target gene that includes the cleavage site. Full complementarity (100%) is not necessarily required, so long as there is sufficient complementarity to cause hybridization and promote formation of a complex, e.g., CRISPR complex, that includes the recombinant nuclease, e.g., Cas9, and the guide RNA, e.g., the first guide RNA, such as the first sgRNA, or the second guide RNA, such as the second sgRNA.

In some embodiments, the cleavage site is situated at a site within the target gene that is homologous to a sequence comprised within the guide RNA, e.g., sgRNA. In some embodiments, the cleavage site of the sense strand is situated at a site within the sense strand of the target gene that is homologous to a sequence comprised within the first guide RNA, e.g., the first sgRNA. In some embodiments, the cleavage site of the antisense strand is situated at a site within the antisense strand of the target gene that is homologous to a sequence comprised within the first guide RNA, e.g., the first sgRNA. In some embodiments, the cleavage site of the sense strand is situated at a site within the sense strand of the target gene that is homologous to a sequence comprised within the second guide RNA, e.g., the second sgRNA. In some embodiments, the cleavage site of the antisense strand is situated at a site within the antisense strand of the target gene that is homologous to a sequence comprised within the second guide RNA, e.g., the second sgRNA. In some embodiments, the cleavage site of the sense strand is situated at a site within the sense strand of the target gene that is homologous to a sequence comprised within the first guide RNA, e.g., the first sgRNA; and the cleavage site of the antisense strand is situated at a site within the antisense strand of the target gene that is homologous to a sequence comprised within the second guide RNA, e.g., the second sgRNA. In some embodiments, the cleavage site of the antisense strand is situated at a site within the antisense strand of the target gene that is homologous to a sequence comprised within the first guide RNA, e.g., the first sgRNA; and the cleavage site of the sense strand is situated at a site within the sense strand of the target gene that is homologous to a sequence comprised within the second guide RNA, e.g., the second sgRNA. In some embodiments, the cleavage site of the antisense strand is situated at a site within the antisense strand of the target gene that is homologous to a sequence comprised within the second guide RNA, e.g., the second sgRNA; and the cleavage site of the sense strand is situated at a site within the sense strand of the target gene that is homologous to a sequence comprised within the first guide RNA, e.g., the first sgRNA.

In some embodiments, the sense strand comprises the targeting sequence, and the targeting sequence includes the SNP and a protospacer adjacent motif (PAM) sequence. In some embodiments, the sense strand comprises the targeting sequence, and the targeting sequence includes the SNP and a protospacer adjacent motif (PAM) sequence; and the antisense strand comprises a sequence that is complementary to the targeting sequence and includes a PAM sequence. In some embodiments, the antisense strand comprises the targeting sequence, and the targeting sequence includes the SNP and a protospacer adjacent motif (PAM) sequence. In some embodiments, the antisense strand comprises the targeting sequence, and the targeting sequence includes the SNP and a protospacer adjacent motif (PAM) sequence; and the sense strand comprises a sequence that is complementary to the targeting sequence and includes a PAM sequence.

In some embodiments, the cleavage site on the sense strand and/or the antisense strand is situated approximately 3 nucleotides upstream of the PAM sequence. In some embodiments, the cleavage site on the sense strand and/or the antisense strand is situated approximately 3 nucleotides upstream of the juncture between the guide RNA and the PAM sequence. In some embodiments, the cleavage site on the sense strand and/or the antisense strand is situated 3 nucleotides upstream of the PAM sequence. In some embodiments, the cleavage site on the sense strand and/or the antisense strand is situated 4 nucleotides upstream of the PAM sequence.

In some embodiments, the PAM sequence that is recognized by a recombinant nuclease is in the sense strand. In some embodiments, the PAM sequence that is recognized by a recombinant nuclease is in the antisense strand. In some embodiments, the PAM sequence that is recognized by a recombinant nuclease is in the sense strand and is in the antisense strand. In some embodiments, the PAM sequence on the sense strand and the PAM sequence on the antisense strand are outwardly facing. In some embodiments, the PAM sequence on the sense strand and the PAM sequence on the antisense strand comprise the same nucleic acid sequence, which can be any PAM sequence disclosed herein. In some embodiments, the PAM sequence on the sense strand and the PAM sequence on the antisense strand each comprise a different nucleic acid sequence, each of which can be any of the PAM sequences disclosed herein.

In some embodiments, the PAM sequence that is recognized by a recombinant nuclease, e.g., Cas9, differs depending on the particular recombinant nuclease and the bacterial species it is from Methods for designing guide RNAs, e.g., sgRNAs, and their exemplary targeting sequences, e.g., crRNA sequences, can include those described in, e.g., International PCT Pub. Nos. WO2015/161276, WO2017/193107, and WO2017/093969. Exemplary guide RNA structures, including particular domains, are described in WO2015/161276, e.g., in FIGS. 1A-1G therein. Since guide RNA is an RNA molecule, it will comprise the base uracil (U), while any DNA encoding the guide RNA molecule will comprise the base thymine (T). In some embodiments, the guide RNA, e.g., sgRNA, comprises a CRISPR targeting RNA sequence (crRNA) and a trans-activating crRNA sequence (tracrRNA). In some embodiments, the first guide RNA, e.g., the first sgRNA, and the second guide RNA, e.g., the second sgRNA, each comprise a crRNA and a tracrRNA. In some embodiments, the guide RNA, e.g., sgRNA, is an RNA comprising, from 5′ to 3′: a crRNA sequence and a tracrRNA sequence. In some embodiments, each of the first guide RNA, e.g., first sgRNA, and the second guide RNA, e.g., second sgRNA, is an RNA comprising, from 5′ to 3′: a crRNA sequence and a tracrRNA sequence. In some embodiments, the crRNA and tracrRNA do not naturally occur together in the same sequence.

In some embodiments, the crRNA comprises a nucleotide sequence that is homologous, e.g., is at least 80%, 85%, 90%, 95%, 96%, 97%, 98%, or 99% homologous, or is 100% homologous, to a portion of the target gene that includes the cleavage site. In some embodiments, the crRNA comprises a nucleotide sequence that is 100% homologous to a portion of the target gene that includes the cleavage site. In some embodiments, the portion of the target gene that includes the cleavage site is a portion of the sense strand of the target gene that includes the cleavage site. In some embodiments, the portion of the target gene that includes the cleavage site is a portion of the antisense strand of the target gene that includes the cleavage site.

In some embodiments, the sgRNA comprises a crRNA sequence that is homologous to a sequence in the target gene that includes the cleavage site. In some embodiments, the first sgRNA comprises a crRNA sequence that is homologous to a sequence in the sense strand of the target gene that includes the cleavage site; and/or the second sgRNA comprises a crRNA sequence that is homologous to a sequence in the antisense strand of the target gene that includes the cleavage site. In some embodiments, the first sgRNA comprises a crRNA sequence that is homologous to a sequence in the antisense strand of the target gene that includes the cleavage site; and/or the second sgRNA comprises a crRNA sequence that is homologous to a sequence in the sense strand of the target gene that includes the cleavage site.

In some embodiments, the crRNA sequence has 100% sequence identity to a sequence in the target gene that includes the cleavage site. In some embodiments, the crRNA sequence of the first sgRNA has 100% sequence identity to a sequence in the sense strand of the target gene that includes the cleavage site; and/or the crRNA sequence of the second sgRNA has 100% sequence identity to a sequence in the antisense strand of the target gene that includes the cleavage site. In some embodiments, the crRNA sequence of the first sgRNA has 100% sequence identity to a sequence in the antisense strand of the target gene that includes the cleavage site; and/or the crRNA sequence of the second sgRNA has 100% sequence identity to a sequence in the sense strand of the target gene that includes the cleavage site.

Guidance on the selection of crRNA sequences can be found, e.g., in Fu Y et al., Nat Biotechnol 2014 (doi: 10.1038/nbt.2808) and Sternberg S H et al., Nature 2014 (doi: 10.1038/nature13011). Examples of the placement of crRNA sequences within the guide RNA, e.g., sgRNA, structure include those described in WO2015/161276, e.g., in FIGS. 1A-1G therein.

Reference to “the crRNA” is to be understood as also including reference to the crRNA of the first sgRNA and the crRNA of the second sgRNA, each independently. Thus, embodiments referring to “the crRNA” is to be understood as independently referring to embodiments of (i) the crRNA, (ii) the crRNA of the first sgRNA, and (iii) the crRNA of the second sgRNA. In some embodiments, the crRNA is 15-27 nucleotides in length, i.e., the crRNA is 15, 16, 17, 18, 19, 20, 21, 22, 23, 24, 25, 26, or 27 nucleotides in length. In some embodiments, the crRNA is 18-22 nucleotides in length. In some embodiments, the crRNA is 19-21 nucleotides in length. In some embodiments, the crRNA is 20 nucleotides in length.

In some embodiments, the crRNA is homologous to a portion of a target gene that includes the cleavage site. In some embodiments, the crRNA is homologous to a portion of the sense strand of the target gene that includes the cleavage site. In some embodiments, the crRNA is homologous to a portion of the antisense strand of the target gene that includes the cleavage site. In some embodiments, the crRNA of the first sgRNA is homologous to a portion of the sense strand of the target gene that includes the cleavage site; and the crRNA of the second sgRNA is homologous to a portion of the antisense strand of the target gene that includes the cleavage site.

In some embodiments, the crRNA is homologous to a portion of the antisense strand of a target gene that includes the cleavage site. In some embodiments, the crRNA is homologous to a portion of the sense strand of the target gene that includes the cleavage site. In some embodiments, the crRNA of the first sgRNA is homologous to a portion of the antisense strand of the target gene that includes the cleavage site; and the crRNA of the second sgRNA is homologous to a portion of the sense strand of the target gene that includes the cleavage site.

In some embodiments, the crRNA is homologous to a portion of a target gene that includes the cleavage site, and is 15-27 nucleotides in length, i.e., the crRNA is 15, 16, 17, 18, 19, 20, 21, 22, 23, 24, 25, 26, or 27 nucleotides in length. In some embodiments, the portion of the target gene that includes the cleavage site is on the sense strand. In some embodiments, the portion of the target gene that includes the cleavage site is on the antisense strand.

In some embodiments, the crRNA is homologous to a portion, i.e., sequence, in the sense strand or the antisense strand of the target gene that includes the cleavage site and is immediately upstream of the PAM sequence.

In some embodiments, the tracrRNA sequence may be or comprise any sequence for tracrRNA that is used in any CRISPR/Cas9 system known in the art. Reference to “the tracrRNA” is to be understood as also including reference to the tracrRNA of the first sgRNA and the tracrRNA of the second sgRNA, each independently. Thus, embodiments referring to “the tracrRNA” is to be understood as independently referring to embodiments of (i) the tracrRNA, (ii) the tracrRNA of the first sgRNA, and (iii) the tracrRNA of the second sgRNA. Exemplary CRISPR/Cas9 systems, sgRNA, crRNA, and tracrRNA, and their manufacturing process and use include those described in, e.g., International PCT Pub. Nos. WO2015/161276, WO2017/193107 and WO2017/093969, and those described in, e.g., U.S. Patent Application Publication Nos. 20150232882, 20150203872, 20150184139, 20150079681, 20150073041, 20150056705,20150031134,20150020223,20140357530,20140335620,20140310830, 20140273234,20140273232,20140273231,20140256046,20140248702,20140242700, 20140242699,20140242664,20140234972,20140227787,20140189896,20140186958, 20140186919, 20140186843, 20140179770, 20140179006, 20140170753, 20140093913, and 20140080216.

In some embodiments, the Cas protein is selected from the group consisting of Cas3, Cas9, Cas10, Cas12, and Cas13. In particular embodiments, the nuclease is a Cas nuclease, such as Cas9. In some embodiments, delivery of the CRISPR/Cas can be used to introduce single point mutations (deletions or insertions) in a particular target gene, via a single gRNA. Using a pair of gRNA-directed Cas9 nucleases instead, it is also possible to induce large deletions or genomic rearrangements, such as inversions or translocations. In some embodiments, a dCas9 version of the CRISPR/Cas9 system can be used to target protein domains for transcriptional regulation, epigenetic modification, and microscopic visualization of specific genome loci.

In some embodiments, the payload agent is an RNA-guided nuclease. In some embodiments, the RNA-guided nuclease is TnpB (see, e.g., WO2018035250 and WO2022159892).

In some embodiments, the payload agent is one for use in target-primed reverse transcription (TPRT) or “prime editing”. In some embodiments, prime editing mediates targeted insertions, deletions, all 12 possible base-to-base conversions, and combinations thereof in human cells without requiring DSBs or donor DNA templates.

Prime editing is a genome editing method that directly writes new genetic information into a specified DNA site using a nucleic acid programmable DNA binding protein (“napDNAbp”) working in association with a polymerase (e.g., in the form of a fusion protein or otherwise provided in trans with the napDNAbp), wherein the prime editing system is programmed with a prime editing (PE) guide RNA (“PEgRNA”) that both specifies the target site and templates the synthesis of the desired edit in the form of a replacement DNA strand by way of an extension (either DNA or RNA) engineered onto a guide RNA (e.g., at the 5′ or 3′ end, or at an internal portion of a guide RNA). The replacement strand containing the desired edit (e.g., a single nucleobase substitution) shares the same sequence as the endogenous strand of the target site to be edited (with the exception that it includes the desired edit). Through DNA repair and/or replication machinery, the endogenous strand of the target site is replaced by the newly synthesized replacement strand containing the desired edit. In some cases, prime editing may be thought of as a “search-and-replace” genome editing technology since the prime editors search and locate the desired target site to be edited, and encode a replacement strand containing a desired edit which is installed in place of the corresponding target site endogenous DNA strand at the same time. For example, prime editing can be adapted for conducting precision CRISPR/Cas-based genome editing in order to bypass double stranded breaks. In some embodiments, the payload agent is a Cas protein-reverse transcriptase fusions or related systems to target a specific DNA sequence with a guide RNA, generate a single strand nick at the target site, and use the nicked DNA as a primer for reverse transcription of an engineered reverse transcriptase template that is integrated with the guide RNA

In some embodiments, the payload agent is a primer editor that is a reverse transcriptase, or any DNA polymerase known in the art. Thus, in one aspect, the prime editor may comprise Cas9 (or an equivalent napDNAbp) which is programmed to target a DNA sequence by associating it with a specialized guide RNA (i.e., PEgRNA) containing a spacer sequence that anneals to a complementary protospacer in the target DNA. Such methods include any disclosed in Anzalone et al., (https://doi.org/10.1038/s41586-019-1711-4), or in PCT publication Nos. WO2020191248, WO2021226558, or WO2022067130, which are hereby incorporated in their entirety.

In some embodiments, the payload agent is for use in Programmable Addition via Site-specific Targeting Elements (PASTE). In some aspects, PASTE is platform in which genomic insertion is directed via a CRISPR-Cas9 nickase fused to both a reverse transcriptase and serine integrase. As described in Loannidi et al. (doi: https://doi.org/10.1101/2021.11.01.466786), PASTE does not generate double stranded breaks, but allowed for integration of sequences as large as ~36 kb. In some embodiments, the serine integrase can be any known in the art. In some embodiments, the serine integrase has sufficient orthogonality such that PASTE can be used for multiplexed gene integration, simultaneously integrating at least two different genes at least two genomic loci. In some embodiments, PASTE has editing efficiencies comparable to or better than those of homology directed repair or non-homologous end joining based integration, with activity in nondividing cells and fewer detectable off-target events.

In some embodiments, the payload agent is associate with base editing. Base editors (BEs) are typically fusions of a Cas (“CRISPR-associated”) domain and a nucleobase modification domain (e.g., a natural or evolved deaminase, such as a cytidine deaminase that include APOBEC1 (“apolipoprotein B mRNA editing enzyme, catalytic polypeptide 1”), CDA (“cytidine deaminase”), and AID (“activation-induced cytidine deaminase”)) domains. In some cases, base editors may also include proteins or domains that alter cellular DNA repair processes to increase the efficiency and/or stability of the resulting single-nucleotide change.

In some embodiments, currently available base editors include cytidine base editors (e.g., BE4) that convert target C⋅G to T⋅A and adenine base editors (e.g., ABE7.10) that convert target A⋅T to G⋅C. In some embodiments, Cas9-targeted deamination was first demonstrated in connection with a Base Editor (BE) system designed to induce base changes without introducing double-stranded DNA breaks. Further Rat deaminase APOBEC1 (rAPOBEC1) fused to deactivated Cas9 (dCas9) was used to successfully convert cytidines to thymidines upstream of the PAM of the sgRNA. In some embodiments, this first BE system was optimized by changing the dCas9 to a “nickase” Cas9 D10A, which nicks the strand opposite the deaminated cytidine. Without being bound by theory, this is expected to initiate long-patch base excision repair (BER), where the deaminated strand is preferentially used to template the repair to produce a U:A base pair, which is then converted to T:A during DNA replication.

In some embodiments, the payload agent is a base editor (e.g., a nucleobase editor). In some embodiments, the payload agent is a nucleobase editor containing a first DNA binding protein domain that is catalytically inactive, a domain having base editing activity, and a second DNA binding protein domain having nickase activity, where the DNA binding protein domains are expressed on a single fusion protein or are expressed separately (e.g., on separate expression vectors). In some embodiments, the base editor is a fusion protein comprising a domain having base editing activity (e.g., cytidine deaminase or adenosine deaminase), and two nucleic acid programmable DNA binding protein domains (napDNAbp), a first comprising nickase activity and a second napDNAbp that is catalytically inactive, wherein at least the two napDNAbp are joined by a linker. In some embodiments, the base editor is a fusion protein that comprises a DNA domain of a CRISPR-Cas (e.g., Cas9) having nickase activity (nCas; nCas9), a catalytically inactive domain of a CRISPR-Cas protein (e.g., Cas9) having nucleic acid programmable DNA binding activity (dCas; e.g., dCas9), and a deaminase domain, wherein the dCas is joined to the nCas by a linker, and the dCas is immediately adjacent to the deaminase domain. In some embodiments, the base editor is a adenine-to-thymine or “ATBE” (or thymine-to-adenine or “TABE”) transversion base editors. Exemplary base editor and base editor systems include any as described in patent publication Nos. US20220127622, US20210079366, US20200248169, US20210093667, US20210071163, WO2020181202, WO2021158921, WO2019126709, WO2020181178, WO2020181195, WO2020214842, WO2020181193, which are hereby incorporated in their entirety.

In some embodiments, the one or more agent(s) (e.g., one or more exogenous agent and/or heterologous protein) capable of inducing a DSB comprise a fusion protein comprising a DNA binding domain and a DNA cleavage domain. In some embodiments, the DNA cleavage domain is or comprises a recombinant nuclease. In some embodiments, the fusion protein is a TALEN comprising a DNA binding domain and a DNA cleavage domain. In some embodiments, the DNA binding domain is a transcription activator-like (TAL) effector DNA binding domain. In some embodiments, the TAL effector DNA binding domain is from Xanthomonas bacteria. In some embodiments, the DNA cleavage domain is a Fok1 nuclease domain. In some embodiments, the TAL effector DNA binding domain is engineered to target a specific target sequence, e.g., a portion of a target gene that includes a cleavage site.

In some embodiments, the fusion protein is a zinc finger nuclease (ZFN) comprising a zinc finger DNA binding domain and a DNA cleavage domain. In some embodiments, the DNA cleavage domain is a Fok1 nuclease domain. In some embodiments, the zinc finger DNA binding domain is engineered to target a specific target sequence, e.g., a portion of a target gene, that includes a cleavage site, such as the targeting sequence.

In some embodiments, a viral vector includes one or more transgenes. In some embodiments, a viral vector includes one, two, three, four, five or more transgenes.

In some embodiments, one or more nucleic acids for the production of the viral vector can include one or more transgenes. In some embodiments, one or more nucleic acids for the production of the viral vector include one, two, three, four, five or more transgenes.

In some embodiments, one or more nucleic acids for the production of the viral vector include a transfer plasmid. In some embodiments, a transfer plasmid can include one or more transgenes. In some embodiments, a transfer plasmid includes one, two, three, four, five or more transgenes.

In some embodiments, a transgene can encode a chimeric antigen receptor (CAR). In some instances, a transgene encoding a CAR can be delivered to, e.g., a T-cell, for expression.

In some embodiments, a T-cell (e.g., a cell to which a transgene encoding a CAR or a CAAR is delivered) is a primary T-cell. In some embodiments a primary T-cell is autologous. In some embodiments, a primary T-cell is allogenic.

In some embodiments, a T-cell is derived from pluripotent stem cells. In some embodiments a pluripotent stem cells is induced (e.g IPS). In some embodiments, a pluripotent stem cell is embryonic.

In some embodiments, a transgene can encode an RNA. For example, a transgene can encode a gRNA, an siRNA, an shRNA, or miRNA.

In some embodiments, a transgene encodes an antibody or portion thereof. Due to size limitations, in some embodiments, a transgene may encode an antibody having an alternative format that is smaller than a full canonical antibody (e.g., a Fab, a diabody, an scFV, a minibody, or nanobody). Viral vectors including transgenes encoding an antibody or portion thereof may be useful in applications involving targeted inhibition of molecules, e.g., molecules associated with specific cell types.

In some embodiments, a transgene encodes an antigen. Viral vectors including such transgenes can be helpful, e.g., in inducing desired immune responses.

In some embodiments, a transgene can encode a therapeutic polypeptide. In some embodiments, a transgene can encode a polypeptide used in protein replacement therapy. In some embodiments, a viral vector can include more than one transgene used in protein replacement therapy. For example, in some embodiments, a viral vector may include a first transgene encoding a nuclease that introduces a genetic modification knocking-out expression of an endogenous (e.g., dysfunctional) polypeptide and a second that delivers a (e.g., functional) replacement protein.

### III. Fusogens

Disclosed herein are methods of producing viral vectors, e.g., lentiviral vectors, comprising one or more fusogens. Also disclosed are one or more nucleic acids for the production of viral vectors (e.g., lentiviral vectors) that comprise one or more nucleic acid sequences encoding a fusogen. In some embodiments, one or more nucleic acids for the production of viral vectors (e.g., lentiviral vectors) comprise one or more envelope plasmids. In some embodiments, one or more envelope plasmids encode one or more fusogens as described herein. In some embodiments, two or more envelope plasmids encode two or more fusogens as described herein.

In some embodiments, the fusogen promotes mixing between lipids in the lentiviral vector and lipids in the target cell. In some embodiments, the fusogen facilitates the fusion of the lentiviral vector to a membrane of the target cell. In some embodiments, the lentiviral vector integrates into the membrane of the target cell. In some embodiments, the membrane is the plasma membrane of the target cell. In some embodiments, the fusogen promotes formation of one or more pores between the interior of the lentiviral vector and the cytosol of the target cell.

In some embodiments, the fusogen is endogenous to the lentiviral vector. In some embodiments, the lentiviral vector is pseudotyped with the fusogen.

In some embodiments, the fusogen is exposed on the surface of the lentiviral vector. In some embodiments, the fusogen is exposed on the surface of the lipid bilayer of the lentiviral vector. In some embodiments, a portion of the fusogen is embedded in the lipid bilayer of the lentiviral vector.

In some embodiments, the fusogen is exposed on the surface of the viral envelope of the lentiviral vector. In some embodiments, a portion of the fusogen is embedded in the viral envelope of the lentiviral vector.

In some embodiments, the fusogen is present at a copy number of at least, or no more than, 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies. In some embodiments, at least 10%, 20%, 30%, 40%, 50%, 60%, 70%, 80%, 90%, 95%, 96%, 97%, 98%, or 99% of the fusogen comprised by the lentiviral vector is disposed in the cell membrane. In some embodiments, the fusogen comprises (or is identified as comprising) about 0.1%, 0.2%, 0.3%, 0.4%, 0.5%, 0.6%, 0.7%, 0.8%, 0.9%, 1%, 5%, 10%, 11%, 12%, 13%, 14%, 15%, 20%, or more, or about 1-30%, 5-20%, 10-15%, 12-15%, 13-14%, or 13.6% of the total protein in a lentiviral vector, e.g., as determined by a mass spectrometry assay. In embodiments, the fusogen comprises (or is identified as comprising) about 13.6% of the total protein in the lentiviral vector. In some embodiments, the fusogen is (or is identified as being) more or less abundant than one or more additional proteins of interest. In an embodiment, the fusogen has (or is identified as having) a ratio to EGFP of about 140, 145, 150, 151, 152, 153, 154, 155, 156, 157 (e.g., 156.9), 158, 159, 160, 165, or 170. In another embodiment, the fusogen has (or is identified as having) a ratio to CD63 of about 2700, 2800, 2900, 2910 (e.g., 2912), 2920, 2930, 2940, 2950, 2960, 2970, 2980, 2990, or 3000, or about 1000-5000, 2000-4000, 2500-3500, 2900-2930, 2910-2915, or 2912.0, e.g., by a mass spectrometry assay. In an embodiment, the fusogen has (or is identified as having) a ratio to ARRDC1 of about 600, 610, 620, 630, 640, 650, 660 (e.g., 664.9), 670, 680, 690, or 700. In another embodiment, the fusogen has (or is identified as having) a ratio to GAPDH of about 50, 55, 60, 65, 70 (e.g., 69), 75, 80, or 85, or about 1-30%, 5-20%, 10-15%, 12-15%, 13-14%, or 13.6%. In another embodiment, the fusogen has (or is identified as having) a ratio to CNX of about 500, 510, 520, 530, 540, 550, 560 (e.g., 558.4), 570, 580, 590, or 600, or about 300-800, 400-700, 500-600, 520-590, 530-580, 540-570, 550-560, or 558.4, e.g., by a mass spectrometry assay.

In some embodiments, the fusogen is a protein fusogen. In some embodiments, the fusogen is a native protein or a derivative of a native protein. In some embodiments, the fusogen is a synthetic protein. In some embodiments, the fusogen is a mammalian protein or a homologue of a mammalian protein (e.g., having 50%, 60%, 70%, 80%, 85%, 90%, 95%, 96%, 97%, 98%, 99%, or greater sequence identity to the mammalian protein). In some embodiments, the fusogen is a non-mammalian protein. In some embodiments, the fusogen is a viral protein or a homologue of a viral protein (e.g., having 50%, 60%, 70%, 80%, 85%, 90%, 95%, 96%, 97%, 98%, 99%, or greater sequence identity to the viral protein). In some embodiments, the fusogen is a fragment of any of the foregoing. In some embodiments, the fusogen is a variant of any of the foregoing. In some embodiments, the fusogen is a protein fusion containing one or more proteins or fragments thereof. In some embodiments, the fusogen is a protein fusion containing one or more of any of the foregoing.

In some embodiments, the fusogen is mutated to reduce binding for the native binding partner of the fusogen. In some embodiments the fusogen is randomly mutated. In some embodiments the fusogen is rationally mutated. In some embodiments the fusogen is subjected to directed evolution. In some embodiments the fusogen is truncated and only a subset of the peptide is used in the lentiviral vector.

### A. Mammalian Proteins

In some embodiments, the fusogen is or contains a mammalian protein. Exemplary mammalian fusogens include a SNARE family protein such as vSNAREs or tSNAREs, a syncytin protein such as Syncytin-1 (DOI: 10.1128/JVI.76.13.6442-6452.2002) and Syncytin-2, myomaker (biorxiv.org/content/early/2017/04/02/123158, doi.org/10.1101/123158, doi: 10.1096/fj.201600945R, doi:10.1038/nature12343), myomixer (www.nature.com/nature/journal/v499/n7458/full/nature12343.html, doi:10.1038/nature12343), myomerger (science.scieencemag.org/content/early/2017/04/05/science.aam9361, DOI: 10.1126/science.aam9361), FGFRL1 (fibroblast growth factor receptor-like 1), Minion (doi.org/10.1101/122697), an isoform of glyceraldehyde-3-phosphate dehydrogenase (GAPDH) (e.g., as disclosed in U.S. Pat. No. 6,099,857A), a gap junction protein such as connexin 43, connexin 40, connexin 45, connexin 32, or connexin 37 (e.g., as disclosed in US 2007/0224176), Hap2, any protein capable of inducing syncytium formation between heterologous cells (see Table 2), any protein with fusogenic properties, a homologue thereof, a fragment thereof, a variant thereof, and a protein fusion containing one or more proteins or fragments thereof, such as one or more of any of the foregoing. In some embodiments, the fusogen is encoded by a human endogenous retroviral element (hERV) found in the human genome. Additional exemplary fusogens are disclosed in U.S. Pat. No. 6,099,857A and US 2007/0224176, the entire contents of each of which are hereby incorporated by reference.

### B. Viral Proteins

In some embodiments, the fusogen is or contains a non-mammalian protein. In some embodiments, the fusogen is or contains a viral protein. In some embodiments, the fusogen is a viral fusion protein. In some embodiments, the fusogen is a viral envelope protein.

In some embodiments, a viral fusogen is a Class I viral membrane fusion protein, a Class II viral membrane protein, a Class III viral membrane fusion protein, a viral membrane glycoprotein, or other viral fusion proteins, or a homologue thereof, a fragment thereof, a variant thereof, or a protein fusion containing one or more proteins or fragments thereof, such as one or more of any of the foregoing.

In some embodiments, Class I viral membrane fusion proteins include Baculovirus F protein, e.g., F proteins of the nucleopolyhedrovirus (NPV) genera, e.g., Spodoptera exigua MNPV (SeMNPV) F protein and Lymantria dispar MNPV (LdMNPV), and paramyxovirus F proteins.

In some embodiments, Class II viral membrane proteins include tick bone encephalitis E (TBEV E) and Semliki Forest Virus E1/E2.

In some embodiments, Class III viral membrane fusion proteins include rhabdovirus G (e.g., fusogenic protein G of the Vesicular Stomatitis Virus (VSV-G), Cocal virus G protein), herpesvirus glycoprotein B (e.g., Herpes Simplex virus 1 (HSV-1) gB), Epstein Barr Virus glycoprotein B (EBV gB), thogotovirus G, baculovirus gp64 (e.g., Autographa California multiple NPV (AcMNPV) gp64), and Borna disease virus (BDV) glycoprotein (BDV G).

Examples of other viral fusogens, e.g., membrane glycoproteins and viral fusion proteins, include viral syncytia proteins such as influenza hemagglutinin (HA) or mutants, or fusion proteins thereof; human immunodeficiency virus type 1 envelope protein (HIV-1 ENV), gp120 from HIV binding LFA-1 to form lymphocyte syncytium, HIV gp41, HIV gp160, or HIV Trans-Activator of Transcription (TAT); viral glycoprotein VSV-G, viral glycoprotein from vesicular stomatitis virus of the Rhabdoviridae family; glycoproteins gB and gH-gL of the varicella-zoster virus (VZV); murine leukaemia virus (MLV)-10A1; Gibbon Ape Leukemia Virus glycoprotein (GaLV); type G glycoproteins in Rabies, Mokola, vesicular stomatitis virus and Togaviruses; murine hepatitis virus JHM surface projection protein; porcine respiratory coronavirus spike- and membrane glycoproteins; avian infectious bronchitis spike glycoprotein and its precursor; bovine enteric coronavirus spike protein; the F and H, HN or G genes of a Morbillivirus (e.g., measles virus (MeV), canine distemper virus, Cetacean morbillivirus, Peste-des-petits-ruminants virus, Phocine distemper virus, Rinderpest virus), Newcastle disease virus, human parainfluenza virus 3, simian virus 41, Sendai virus and human respiratory syncytial virus; gH of human herpesvirus 1 and simian varicella virus, with the chaperone protein gL; human, bovine and cercopithicine herpesvirus gB; envelope glycoproteins of Friend murine leukaemia virus and Mason Pfizer monkey virus; mumps virus hemagglutinin neuraminidase, and glycoproteins F1 and F2; membrane glycoproteins from Venezuelan equine encephalomyelitis; paramyxovirus F protein; SIV gp160 protein; Ebola virus G protein; or Sendai virus fusion protein, or a homologue thereof, a fragment thereof, a variant thereof, and a protein fusion containing one or more proteins or fragments thereof, such as one or more of any of the foregoing.

Non-mammalian fusogens include viral fusogens, homologues thereof, fragments thereof, and fusion proteins comprising one or more proteins or fragments thereof, such as one or more of any of the foregoing. Viral fusogens include class I fusogens, class II fusogens, class III fusogens, and class IV fusogens. In some embodiments, class I fusogens such as human immunodeficiency virus (HIV) gp41 have a characteristic postfusion conformation with a signature trimer of α-helical hairpins with a central coiled-coil structure. Class I viral fusion proteins include proteins having a central postfusion six-helix bundle. Class I viral fusion proteins include influenza HA, parainfluenza F, HIV Env, Ebola GP, hemagglutinins from orthomyxoviruses, F proteins from paramyxoviruses (e.g. Measles, (Katoh et al. BMC Biotechnology 2010, 10:37)), ENV proteins from retroviruses, and fusogens of filoviruses and coronaviruses. In some embodiments, class II viral fusogens such as dengue E glycoprotein, have a structural signature of β-sheets forming an elongated ectodomain that refolds to result in a trimer of hairpins. In some embodiments, the class II viral fusogen lacks the central coiled coil. Class II viral fusogen can be found in alphaviruses (e.g., E1 protein) and flaviviruses (e.g., E glycoproteins). Class II viral fusogens include fusogens from Semliki Forest virus, Sinbis, rubella virus, and dengue virus. In some embodiments, class III viral fusogens such as the vesicular stomatitis virus G glycoprotein, combine structural signatures found in classes I and II. In some embodiments, a class III viral fusogen comprises a helices (e.g., forming a six-helix bundle to fold back the protein as with class I viral fusogens), and p sheets with an amphiphilic fusion peptide at its end, reminiscent of class II viral fusogens. Class III viral fusogens can be found in rhabdoviruses and herpesviruses. In some embodiments, class IV viral fusogens are fusion-associated small transmembrane (FAST) proteins (doi:10.1038/sj.emboj.7600767, Nesbitt, Rae L., “Targeted Intracellular Therapeutic Delivery Using Liposomes Formulated with Multifunctional FAST proteins” (2012). Electronic Thesis and Dissertation Repository. Paper 388), which are encoded by nonenveloped reoviruses. In some embodiments, the class IV viral fusogens are sufficiently small that they do not form hairpins (doi: 10.1146/annurev-cellbio-101512-122422, doi:10.1016/j.devcel.2007.12.008).

In some embodiments, the fusogen is any of the fusogenic moieties described in WO2017/182585; WO2022/164935; WO2021/076788; Hamilton et al. bioRxiv 2022.08.24.505004; Nikolic et al. Nat Commun 9, 1029 (2018); Dobson et al. Nat. Methods. 19, 449-460 (2022); and Yu et al. bioRxiv 2021.12.13.472464, for instance any of the VSV or variant VSV glycoproteins described therein, such as VSV glycoproteins that have reduced binding to native receptors.

### 1. Baboon Endogenous Retrovirus

In some embodiments, the fusogen is a Baboon Endogenous Retrovirus (BaEV) envelope glycoprotein. Exemplary BaEV envelope glycoproteins and variants thereof are described in PCT/US2022/031459; U.S. Pat. No. 9,249,426; Aguila et al. Journal of Virology 2003 77(2):1281-1291; Bernadin et al. Blood Advances 2019 3(3):461-475; Colamartino et al. Frontiers in Immunology 2019 10:2873; Girard-Gagnepain et al. Blood 2014 124(8):1221-1231; and Levy et al. Journal of Thrombosis and Haemostasis 2016 14:2478-2492.

Wild-type BaEV envelope glycoproteins are retroviral envelope proteins containing a C-terminal cytoplasmic tail (e.g., corresponding to amino acids 512-545 of SEQ ID NO:252), a transmembrane domain (e.g., corresponding to amino acids 489-511 of SEQ ID NO:252), and an extracellular domain (e.g., corresponding to amino acids 1-488 of SEQ ID NO:252). Maturation of the precursor protein in the Golgi, which requires the minimal sequence [KR]-X-[KR]-R (wherein X is any amino acid), results in two subunits, the surface unit protein or gp70, and the transmembrane protein p20E. The surface unit protein or gp70 (e.g., corresponding to amino acids 1-358 of SEQ ID NO:252) and the transmembrane protein p20E (e.g., corresponding to amino acids 359-545 of SEQ ID NO:252) remain associated in a labile interaction that may include a disulfide bond. In wild-type BaEV envelope glycoproteins, fusogenicity is controlled by a short, 17 amino acid sequence termed a fusion inhibitory R peptide (e.g., set forth in SEQ ID NO:253), which is localized on the C-terminal of the cytoplasmic tail domain. The fusion inhibitory R peptide harbors the tyrosine endocytosis signal YXXL, and its cleavage by the viral protease is thought to potentiate fusogenic activation through molecular rearrangements in the membrane-spanning domain and the extracellular region of the envelope glycoprotein (Salamango et al (2015) Journal of virology 89(24):12492-12500). In wild-type BaEV envelope glycoproteins, the gp70 mediates receptor binding to the ASCT-2 and ASCT-1 receptors on host cells. In some embodiments, the glycoprotein 70 (g70) subunit or a biologically active portion thereof binds the ASCT-2 and ASCT-1 receptors. In wild-type BaEV envelope glycoproteins, the p20E acts as a class I viral fusion protein. The interaction of the gp70 subunit with a host cell membrane triggers refolding of the p20E and is believed to activate the fusogenic potential by unmasking the fusion peptide.

In some embodiments, the fusogen is a baboon endogenous virus (BaEV) envelope glycoprotein or a functional variant thereof.

In some embodiments, the fusogen is a truncated BaEV envelope glycoprotein. Exemplary BaEV envelope glycoproteins and truncates thereof are described in PCT/US2022/031459. In some embodiments, the truncated BaEV envelope glycoprotein comprises a cytoplasmic tail with a partial fusion inhibitory R peptide relative to a wild-type BaEV envelope glycoprotein, wherein the R peptide contains a contiguous portion of the inhibitory R peptide but lacks the full length R peptide of wild-type BaEV envelope glycoprotein. In some embodiments, the truncated BaEV envelope glycoprotein has a cytoplasmic tail that is composed of a partial inhibitory R peptide with at least one, at least two, or at least three contiguous amino-terminal amino acids of the inhibitory R peptide but less than the full-length R peptide relative to wild-type BaEV envelope glycoprotein. In some embodiments, the truncated BaEV envelope glycoprotein has a cytoplasmic tail that has a partial inhibitory R peptide composed of 1 to 16 contiguous amino-terminal amino acids of the inhibitory R peptide of the wild-type BaEV envelope glycoprotein, such as is composed of 1, 2, 3, 4, 5, 6, 7, 8, 9, 10, 11, 12, 12, 14, 15 or 16 amino-terminal amino acids of the inhibitory R peptide of the wild-type BaEV envelope glycoprotein. In some embodiments, the truncated BaEV envelope glycoprotein is set forth in any of SEQ ID NO:254-260. In some embodiments, the truncated BaEV envelope glycoprotein s BaEV R+8 set fort hin SEQ ID NO:259.

In some embodiments, the fusogen is a modified BaEV envelope glycoprotein. In some embodiments, the cytoplasmic tail domain of the BaEV envelope glycoprotein is devoid of the fusion inhibitory R peptide. The expression “fusion inhibitory R peptide” refers to the C-terminal portion of the cytoplasmic tail domain of the envelope glycoprotein which harbours a tyrosine endocytosis signal—YXXL—and which is cleaved by viral protease during virion maturation, thus enhancing membrane fusion of the envelope glycoprotein. The fusion inhibitory R peptide of the BaEV envelope glycoprotein is typically located between amino acids 547 and 564 of the wild-type BaEV envelope glycoprotein. In some embodiments, the modified BaEV envelope glycoprotein is set forth in SEQ ID NO: 261 (BaEVRLess).

In some embodiments, the cytoplasmic tail domain of the BaEV envelope glycoprotein is replaced by the cytoplasmic tail domain of a murine leukemia virus (MLV) envelope glycoprotein. The Murine Leukemia Virus envelope glycoprotein is notably described in Ott et al. (1990) J. Virol. 64:757-766. In some embodiments, the Murine Leukemia Virus envelope glycoprotein is that of strain 4070A. The term “MLV envelope glycoprotein” refers to the wild-type form of the MLV envelope glycoprotein or to a mutant of said wild-type MLV envelope glycoprotein which is at least 80%, preferably at least 85%, still preferably at least 90%, more preferably at least 95%, still more preferably at least 99% identical to said wild-type MLV envelope glycoprotein, provided that said mutant glycoprotein retains the capacity of the wild-type envelope glycoprotein of interacting with viral core proteins, in particular with lentiviral core proteins. Typically, the cytoplasmic tail domain of the MLV envelope glycoprotein is located between amino acids 622 and 654 of the wild-type MLV envelope glycoprotein. In some embodiments, the fusogen is BaEVTR. In some embodiments, the modified BaEV envelope glycoprotein is set forth in SEQ ID NO: 262 (BaEVTR).

In some embodiments where a VLP includes a BaEVTR glycoprotein fusogen (e.g., a fusogen comprising a baboon endogenous virus (BaEV) envelope glycoprotein or a functional variant thereof or a truncated BaEV envelope glycoprotein or a functional variant thereof), the VLP also comprises a gag protein (e.g. an MLV-gag protein). In some embodiments, the MLV-gag protein is part of a fusion protein, where the the fusion protein is a cleavable fusion protein between (i) a viral structural protein (e.g. GAG (e.g. MLV-gag or HIV-gag)) and (ii) a nuclease protein (e.g. Cas protein (e.g. any of the Cas protein described herein). In some embodiments where the VLP includes a BaEVTR glycoprotein fusogen and a gag protein (e.g. an MLV-gag protein), production of such a VLP includes varying the ratio of polynucleotide encoding the BaEVTR glycoprotein fusogen and the polynucleotide encoding the gag protein. In some embodiments, the ratio of polynucleotide encoding the BaEVTR glycoprotein fusogen to the polynucleotide encoding the gag protein includes a 1:1, 1:2, 1:3, 1:4, or 1:5 ratio.

### 2. G/H Proteins

In some embodiments, the fusogen is or contains a G or H protein. In some embodiments, the G or H protein is a Paramyxovirus (e.g., Morbillivirus or Henipavirus) G or H protein or a biologically active portion thereof. In some embodiments, the Henipavirus G protein is a Hendra (HeV) virus G protein, a Nipah (NiV) virus G-protein (NiV-G), a Cedar (CedPV) virus G-protein, a Mojiang virus G-protein, a bat Paramyxovirus G-protein, a Kumasi virus G-protein, a Langya virus G-protein, or a biologically active portion thereof. A non-limited list of exemplary G proteins is shown in Table 24.

The Henipavirus attachment G proteins are type II transmembrane glycoproteins containing an N-terminal cytoplasmic tail (e.g., corresponding to amino acids 1-49 of SEQ ID NO:1), a transmembrane domain (e.g., corresponding to amino acids 50-70 of SEQ ID NO:1, and an extracellular domain containing an extracellular stalk (e.g., corresponding to amino acids 71-187 of SEQ ID NO:1), and a globular head (corresponding to amino acids 188-602 of SEQ ID NO:1). The N-terminal cytoplasmic domain is within the inner lumen of the lipid bilayer and the C-terminal portion is the extracellular domain that is exposed on the outside of the lipid bilayer. Regions of the stalk in the C-terminal region (e.g. corresponding to amino acids 159-167 of NiV-G) have been shown to be involved in interactions with F protein and triggering of F protein fusion (Liu et al. 2015 J of Virology 89:1838). In wild-type NiV-G protein, the globular head mediates receptor binding to henipavirus entry receptors ephrin B2 and ephrin B3, but is dispensable for membrane fusion (Brande-Tretheway et al. Journal of Virology. 2019. 93(13)e00577-19).

In particular embodiments herein, tropism of the G protein is modified. Binding of the G protein to a binding partner can trigger fusion mediated by a compatible F protein or biologically active portion thereof. G protein sequences disclosed herein are predominantly disclosed as expressed sequences including an N-terminal methionine required for start of translation. As such N-terminal methionines are commonly cleaved co- or post-translationally, the mature protein sequences for all G protein sequences disclosed herein are also contemplated as lacking the N-terminal methionine.

G glycoproteins are highly conserved between henipavirus species. For example, the G protein of NiV and HeV viruses share 79% amino acids identity. Studies have shown a high degree of compatibility among G proteins with F proteins of different species as demonstrated by heterotypic fusion activation (Brandel-Tretheway et al. Journal of Virology. 2019). As described below, a re-targeted lentiviral vector can contain heterologous proteins from different species.

TABLE 24
Exemplary Henipavirus G Proteins

| | | | SEQ ID NO |
|---|---|---|---|
| | | | (without |
| Viral G | | | N-terminal |
| Protein | Sequence | SEQ ID NO: | methionine) |
| Hendra Virus | MMADSKLVSLNNNLSGKIKDQGKVIKNYYG | 2 | 3 |
| G Protein | TMDIKKINDGLLDSKILGAFNTVIALLGSIIIIV | | |
| | MNIMIIQNYTRTTDNQALIKESLQSVQQQIKA | | |
| | LTDKIGTEIGPKVSLIDTSSTITIPANIGLLGSKI | | |
| | SQSTSSINENVNDKCKFTLPPLKIHECNISCPN | | |
| | PLPFREYRPISQGVSDLVGLPNQICLQKTTSTI | | |
| | LKPRLISYTLPINTREGVCITDPLLAVDNGFFA | | |
| | YSHLEKIGSCTRGIAKQRIIGVGEVLDRGDKV | | |
| | PSMFMTNVWTPPNPSTIHHCSSTYHEDFYYT | | |
| | LCAVSHVGDPILNSTSWTESLSLIRLAVRPKSD | | |
| | SGDYNQKYIAITKVERGKYDKVMPYGPSGIK | | |
| | QGDTLYFPAVGFLPRTEFQYNDSNCPIIHCKYS | | |
| | KAENCRLSMGVNSKSHYILRSGLLKYNLSLG | | |
| | GDIILQFIEIADNRLTIGSPSKIYNSLGQPVFYQ | | |
| | ASYSWDTMIKLGDVDTVDPLRVQWRNNSVI | | |
| | SRPGQSQCPRFNVCPEVCWEGTYNDAFLIDR | | |
| | LNWVSAGVYLNSNQTAENPVFAVFKDNEILY | | |
| | QVPLAEDDTNAQKTITDCFLLENVIWCISLVEI | | |
| | YDTGDSVIRPKLFAVKIPAQCSES | | |
| Nipah Virus G | MPAENKKVRFENTTSDKGKIPSKVIKSYYGT | 4 | 5 |
| Protein | MDIKKINEGLLDSKILSAFNTVIALLGSIVIIV | | |
| | MNIMIIQNYTRSTDNQAVIKDALQGIQQQIKG | | |
| | LADKIGTEIGPKVSLIDTSSTITIPANIGLLGSKI | | |
| | SQSTASINENVNEKCKFTLPPLKIHECNISCPN | | |
| | PLPFREYRPQTEGVSNLVGLPNNICLQKTSNQ | | |
| | ILKPKLISYTLPVVGQSGTCITDPLLAMDEGY | | |
| | FAYSHLERIGSCSRGVSKQRIIGVGEVLDRGD | | |
| | EVPSLFMTNVWTPPNPNTVYHCSAVYNNEFY | | |
| | YVLCAVSTVGDPILNSTYWSGSLMMTRLAVK | | |
| | PKSNGGGYNQHQLALRSIEKGRYDKVMPYG | | |
| | PSGIKQGDTLYFPAVGFLVRTEFKYNDSNCPIT | | |
| | KCQYSKPENCRLSMGIRPNSHYILRSGLLKYN | | |
| | LSDGENPKVVFIEISDQRLSIGSPSKIYDSLGQ | | |
| | PVFYQASFSWDTMIKFGDVLTVNPLVVNWR | | |
| | NNTVISRPGQSQCPRFNTCPEICWEGVYNDAF | | |
| | LIDRINWISAGVFLDSNQTAENPVFTVFKDNEI | | |
| | LYRAQLASEDTNAQKTITNCFLLKNKIWCISL | | |
| | VEIYDTGDNVIRPKLFAVKIPEQCT | | |
| Cedar Virus G | MLSQLQKNYLDNSNQQGDKMNNPDKKLSV | 6 | 7 |
| Protein | NFNPLELDKGQKDLNKSYYVKNKNYNVSNL | | |
| | LNESLHDIKFCIYCIFSLLIIITIINIITISIVITRLK | | |
| | VHEENNGMESPNLQSIQDSLSSLTNMINTEITP | | |
| | RIGILVTATSVTLSSSINYVGTKTNQLVNELKD | | |
| | YITKSCGFKVPELKLHECNISCADPKISKSAM | | |
| | YSTNAYAELAGPPKIFCKSVSKDPDFRLKQID | | |
| | YVIPVQQDRSICMNNPLLDISDGFFTYIHYEGI | | |
| | NSCKKSDSFKVLLSHGEIVDRGDYRPSLYLLS | | |
| | SHYHPYSMQVINCVPVTCNQSSFVFCHISNNT | | |
| | KTLDNSDYSSDEYYITYFNGIDRPKTKKIPINN | | |
| | MTADNRYIHFTFSGGGGVCLGEEFIIPVTTVIN | | |
| | TDVFTHDYCESFNCSVQTGKSLKEICSESLRS | | |
| | PTNSSRYNLNGIMIISQNNMTDFKIQLNGITY | | |
| | NKLSFGSPGRLSKTLGQVLYYQSSMSWDTYL | | |
| | KAGFVEKWKPFTPNWMNNTVISRPNQGNCP | | |
| | RYHKCPEICYGGTYNDIAPLDLGKDMYVSVI | | |
| | LDSDQLAENPEITVENSTTILYKERVSKDELNT | | |
| | RSTTTSCFLFLDEPWCISVLETNRFNGKSIRPE | | |
| | IYSYKIPKYC | | |
| Bat | MPQKTVEFINMNSPLERGVSTLSDKKTLNQS | 8 | 9 |
| Paramyxovirus | KITKQGYFGLGSHSERNWKKQKNQNDHYMT | | |
| G Protein, | VSTMILEILVVLGIMENLIVLTMVYYQNDNIN | | |
| Eid_hel/GH- | QRMAELTSNITVLNLNLNQLTNKIQREIIPRIT | | |
| M74a/GHA/2009 | LIDTATTITIPSAITYILATLTTRISELLPSINQKC | | |
| | EFKTPTLVLNDCRINCTPPLNPSDGVKMSSLA | | |
| | TNLVAHGPSPCRNFSSVPTIYYYRIPGLYNRTA | | |
| | LDERCILNPRLTISSTKFAYVHSEYDKNCTRGF | | |
| | KYYELMTFGEILEGPEKEPRMFSRSFYSPTNA | | |
| | VNYHSCTPIVTVNEGYFLCLECTSSDPLYKAN | | |
| | LSNSTFHLVILRHNKDEKIVSMPSFNLSTDQE | | |
| | YVQIIPAEGGGTAESGNLYFPCIGRLLHKRVTH | | |
| | PLCKKSNCSRTDDESCLKSYYNQGSPQHQVV | | |
| | NCLIRIRNAQRDNPTWDVITVDLTNTYPGSRS | | |
| | RIFGSFSKPMLYQSSVSWHTLLQVAEITDLDK | | |
| | YQLDWLDTPYISRPGGSECPFGNYCPTVCWE | | |
| | GTYNDVYSLTPNNDLFVTVYLKSEQVAENPY | | |
| | FAIFSRDQILKEFPLDAWISSARTTTISCFMFNN | | |
| | EIWCIAALEITRLNDDIIRPIYYSFWLPTDCRTP | | |
| | YPHTGKMTRVPLRSTYNY | | |
| Mojiang virus, | MATNRDNTITSAEVSQEDKVKKYYGVETAEK | 10 | 11 |
| Tongguan 1 G | VADSISGNKVFILMNTLLILTGAIITITLNITNLT | | |
| Protein | AAKSQQNMLKIIQDDVNAKLEMFVNLDQLV | | |
| | KGEIKPKVSLINTAVSVSIPGQISNLQTKFLQK | | |
| | YVYLEESITKQCTCNPLSGIFPTSGPTYPPTDK | | |
| | PDDDTTDDDKVDTTIKPIEYPKPDGCNRTGD | | |
| | HFTMEPGANFYTVPNLGPASSNSDECYTNPSF | | |
| | SIGSSIYMFSQEIRKTDCTAGEILSIQIVLGRIV | | |
| | DKGQQGPQASPLLVWAVPNPKIINSCAVAAGD | | |
| | EMGWVLCSVTLTAASGEPIPHMFDGFWLYKL | | |
| | EPDTEVVSYRITGYAYLLDKQYDSVFIGKGG | | |
| | GIQKGNDLYFQMYGLSRNRQSFKALCEHGSC | | |
| | LGTGGGGYQVLCDRAVMSFGSEESLITNAYL | | |
| | KVNDLASGKPVIIGQTFPPSDSYKGSNGRMY | | |
| | TIGDKYGLYLAPSSWNRYLRFGITPDISVRSTT | | |
| | WLKSQDPIMKILSTCTNTDRDMCPEICNTRG | | |
| | YQDIFPLSEDSEYYTYIGITPNNGGTKNFVAVR | | |
| | DSDGHIASIDILQNYYSITSATISCFMYKDEIW | | |
| | CIAITEGKKQKDNPQRIYAHSYKIRQMCYNM | | |
| | KSATVTVGNAKNITIRRY | | |

In some embodiments, the G protein has a sequence set forth in any of SEQ ID NOs: 1-11 or is a functionally active variant or biologically active portion thereof that has a sequence that is at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identical to any one of SEQ ID NOs:1, 2, 3, 4, 5, 6, 7, 8, 9, 10 or 11. In some embodiments, the G protein has a sequence set forth in SEQ ID NO:1 or is a functionally active variant or biologically active portion thereof that has a sequence that is at least at or about 80%, at least at or about 90%, at least at or about 95%, or at least at or about 99% identical to SEQ ID NO:1. In some embodiments, the G protein has a sequence set forth in SEQ ID NO:4 or is a functionally active variant or biologically active portion thereof that has a sequence that is at least at or about 80%, at least at or about 90%, at least at or about 95%, or at least at or about 99% identical to SEQ ID NO:4. In some embodiments, the G protein has a sequence set forth in SEQ ID NO:5 or is a functionally active variant or biologically active portion thereof that has a sequence that is at least at or about 80%, at least at or about 90%, at least at or about 95%, or at least at or about 99% identical to SEQ ID NO:5.

In particular embodiments, the G protein or functionally active variant or biologically active portion is a protein that retains fusogenic activity in conjunction with a Henipavirus F protein, e.g. NiV-F or HeV-F. Fusogenic activity includes the activity of the G protein in conjunction with a Henipavirus F protein to promote or facilitate fusion of two membrane lumens, such as the lumen of the targeted viral vector having embedded in its lipid bilayer a henipavirus F and G protein, and a cytoplasm of a target cell, e.g. a cell that contains a surface receptor or molecule that is recognized or bound by the targeted envelope protein. In some embodiments, the F protein and G protein are from the same Henipavirus species (e.g. NiV-G and NiV-F). In some embodiments, the F protein and G protein are from different Henipavirus species (e.g. NiV-G and HeV-F).

In particular embodiments, the G protein has the sequence of amino acids set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10 or SEQ ID NO:11 or is a functionally active variant thereof or a biologically active portion thereof that retains fusogenic activity. In some embodiments, the functionally active variant comprises an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10 or SEQ ID NO: 11 and retains fusogenic activity in conjunction with a Henipavirus F protein (e.g., NiV-F or HeV-F). In some embodiments, the biologically active portion has an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10 or SEQ ID NO:11 and retains fusogenic activity in conjunction with a Henipavirus F protein (e.g., NiV-F or HeV-F).

Reference to retaining fusogenic activity includes activity (in conjunction with a Henipavirus F protein) that is between at or about 10% and at or about 150% or more of the level or degree of binding of the corresponding wild-type G protein, such as set forth in SEQ ID NO: 1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10 or SEQ ID NO:11 such as at least or at least about 10% of the level or degree of fusogenic activity of the corresponding wild-type G protein, such as at least or at least about 15% of the level or degree of fusogenic activity of the corresponding wild-type G protein, such as at least or at least about 20% of the level or degree of fusogenic activity of the corresponding wild-type G protein, such as at least or at least about 25% of the level or degree of fusogenic activity of the corresponding wild-type G protein, such as at least or at least about 30% of the level or degree of fusogenic activity of the corresponding wild-type G protein, such as at least or at least about 35% of the level or degree of fusogenic activity of the corresponding wild-type G protein, such as at least or at least about 40% of the level or degree of fusogenic activity of the corresponding wild-type G protein, such as at least or at least about 45% of the level or degree of fusogenic activity of the corresponding wild-type G protein, such as at least or at least about 50% of the level or degree of fusogenic activity of the corresponding wild-type G protein, such as at least or at least about 55% of the level or degree of fusogenic activity of the corresponding wild-type G protein, such as at least or at least about 60% of the level or degree of fusogenic activity of the corresponding wild-type G protein, such as at least or at least about 65% of the level or degree of fusogenic activity of the corresponding wild-type G protein, such as at least or at least about 70% of the level or degree of fusogenic activity of the corresponding wild-type G protein, such as at least or at least about 75% of the level or degree of fusogenic activity of the corresponding wild-type G protein, such as at least or at least about 80% of the level or degree of fusogenic activity of the corresponding wild-type G protein, such as at least or at least about 85% of the level or degree of fusogenic activity of the corresponding wild-type G protein, such as at least or at least about 90% of the level or degree of fusogenic activity of the corresponding wild-type G protein, such as at least or at least about 95% of the level or degree of fusogenic activity of the corresponding wild-type G protein, such as at least or at least about 100% of the level or degree of fusogenic activity of the corresponding wild-type G protein, or such as at least or at least about 120% of the level or degree of fusogenic activity of the corresponding wild-type G protein.

### 3. Mutated Paramyxovirus G/H Proteins

In some embodiments the G protein is a mutant G protein that is a functionally active variant or biologically active portion containing one or more amino acid mutations, such as one or more amino acid insertions, deletions, substitutions or truncations. In some embodiments, the mutations described herein relate to amino acid insertions, deletions, substitutions or truncations of amino acids compared to a reference G protein sequence. In some embodiments, the reference G protein sequence is the wild-type sequence of a G protein or a biologically active portion thereof. In some embodiments, the functionally active variant or the biologically active portion thereof is a mutant of a wild-type Hendra (HcV) virus G protein, a wild-type Nipah (NiV) virus G-protein (NiV-G), a wild-type Cedar (CedPV) virus G-protein, a wild-type Mojiang virus G-protein, a wild-type bat Paramyxovirus G-protein, a Kumasi virus G-protein, a Langya virus G-protein, or biologically active portion thereof. In some embodiments, the wild-type G protein has the sequence set forth in any one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10 or SEQ ID NO:11.

Provided herein are mutant Paramyxovirus G/H glycoproteins (e.g., variant Paramyxovirus G/H glycoproteins) comprising one or more amino acid mutations that result in decreased glycosylation of the protein. The one or more amino acid mutations, also called deglycosylation mutations, can be one or more amino acid substitutions (also referred to as mutations).

In some embodiments, the mutant Paramyxovirus G/H glycoprotein comprises an amino acid substitution at one or more amino acid positions that reduce glycosylation of the G/H glycoprotein. In some embodiments, the one or more amino acid substitutions disrupts an N-linked glycosylation site. In some embodiments, the one or more amino acid substitutions disrupts an O-linked glycosylation site.

In some embodiments, the mutant Paramyxovirus G/H glycoprotein is derived from Morbillivirus (e.g., measles virus (MeV), canine distemper virus, Cetacean morbillivirus, Peste-des-petits-ruminants virus, Phocine distemper virus, Rinderpest virus), Henipavirus (e.g., Hendra (HeV) virus, Nipah (NiV) virus, a Cedar (CedPV) virus, Mojiang virus, a Langya virus or bat Paramyxovirus). In some embodiments, the mutant Paramyxovirus G/H glycoprotein is a mutant of a Paramyxovirus G/H glycoprotein derived from Nipah virus or Measles virus. In some embodiments, the mutant Paramyxovirus G/H protein is selected from the group consisting of SEQ ID NOs: 2-11, 375, and 376, or a modified Paramyxovirus G/H glycoprotein derived from any one of 2-11, 375, and 376 containing an altered cytoplasmic tail. In some embodiments, the mutant Paramyxovirus G/H protein has a sequence of amino acids that has at least 85%, at least 86%, at least 87%, at least 88%, at least 89%, at least 90%, at least 91%, at least 92%, at least 93%, at least 94% or at least 95% to any one of SEQ ID NOs: 2-11, 375, and 376 and contains the acid substitution at one or more amino acid positions that reduce glycosylation of the G/H glycoprotein as provided herein.

The location of precited glycosylation sites can be determined using the sequence of a protein. For example, N-glycosylation often occurs at sites with the sequence N-X-S/T in which “X” is any amino acid except P. Various algorithms and tools are available for prediction of both N- and O-linked glycosylation, including SprintGly (http://sparks-lab.org/server/sprint-gly/), NetNGlyc (services.healthtech.dtu.dk/service.php?NetNGlyc-1.0), NetOGlyc (services.healthtech.dtu.dk/service.php?NetOGlyc-4.0), and GlycoMine^{struct} (glycomine.erc.monash.edu/Lab/GlycoMine_Struct/), and methods described in Pitti et al., Sci. Reports, 9:15975 (2019) and Pakhrin et al., Molecules 26:7314 (2021). Any predicted glycosylation site may be substituted as described herein.

In some embodiments, the Paramyxovirus G/H glycoprotein to which the deglycosylation mutation is made is a NiV-G set forth in SEQ ID NO: 4 or a modified Nipah G glycoprotein (NiV-G) that has an altered cytoplasmic tail compared to native NiV-G (e.g., SEQ ID NO: 4). In some embodiments, the variant Paramyxovirus G/H protein has a sequence of amino acids that has at least 85%, at least 86%, at least 87%, at least 88%, at least 89%, at least 90%, at least 91%, at least 92%, at least 93%, at least 94% or at least 95% to SEQ ID NO: 4 and contains the acid substitution at one or more amino acid positions that reduce glycosylation of the G/H glycoprotein as provided herein.

In some embodiments, the one or more amino acid mutations are at positions corresponding to positions 39, 126, 128, 273, 345, 384, 448, and 496 of SEQ ID NO:42. In some embodiments, the variant Paramyxovirus G/H glycoprotein comprises an amino acid mutation at any one of positions 39, 126, 128, 273, 345, 384, 448, and 496 of SEQ ID NO:42. In some embodiments, the variant Paramyxovirus G/H glycoprotein comprises two or more amino acid mutations at any of positions corresponding to positions 39, 126, 128, 273, 345, 384, 448, and 496 of SEQ ID NO:42., such as mutations at 2, 3, 4, 5, 7, or 8 of the positions.

In some embodiments, the one or more amino acid mutations is at a position corresponding to position 39 of SEQ ID NO:42. In some embodiments, the one or more amino acid mutations is at a position corresponding to position 126 of SEQ ID NO:42. In some embodiments, the one or more amino acid mutations is at a position corresponding to position 128 of SEQ ID NO:42. In some embodiments, the one or more amino acid mutations is at a position corresponding to position 273 of SEQ ID NO:42. In some embodiments, the one or more amino acid mutations is at a position corresponding to position 345 of SEQ ID NO:42. In some embodiments, the one or more amino acid mutations is at a position corresponding to position 384 of SEQ ID NO:42. In some embodiments, the one or more amino acid mutations is at a position corresponding to position 448 of SEQ ID NO:42. In some embodiments, the one or more amino acid mutations is at a position corresponding to position 496 of SEQ ID NO:42.

In some embodiments, the native amino acid at the position comprising the amino acid mutation is asparagine or serine. In some embodiments, the amino acid mutation is an amino acid substitution. In some embodiments, the mutation is an asparagine to glutamine substitution. In some embodiments, the mutation is a serine to alanine substitution.

In some embodiments, the mutation is an asparagine to glutamine substitution at a position corresponding to position 39 (N39Q) of SEQ ID NO:42. In some embodiments, the mutation is an asparagine to glutamine substitution at a position corresponding to position 126 (N126Q) of SEQ JD NO: 42. In some embodiments, the mutation is an asparagine to glutamine substitution at a position corresponding to position 273 (N273Q) of SEQ ID NO: 42. In some embodiments, the mutation is an asparagine to glutamine substitution at a position corresponding to position 345 (N345Q) of SEQ ID NO: 42. In some embodiments, the mutation is an asparagine to glutamine substitution at a position corresponding to position 384 (N384Q) of SEQ ID NO: 42. In some embodiments, the mutation is an asparagine to glutamine substitution at a position corresponding to position 448 (N448Q) of SEQ ID NO: 42. In some embodiments, the mutation is an asparagine to glutamine substitution at a position corresponding to position 496 (N496Q) of SEQ ID NO: 42.

In some embodiments, the mutation is a serine to alanine substitution at a position corresponding to position 128 (S128A) of SEQ ID NO: 42.

In some embodiments, the G/H glycoprotein is derived from Nipah virus G protein and the one or more amino acid substitutions are at positions corresponding to positions selected from the group consisting of 39, 126, 128, 273, 345, 384, 448, and 496 of SEQ ID NO: 42. In some embodiments, the one or more amino acid substitutions are selected from N39Q, N126Q, S128A, N273Q, N345Q, N384Q, N448Q, N496Q or any combination thereof. In some embodiments, the G/H glycoprotein is a mutant NiV-G containing one amino acid substitution from any one of N39Q, N126Q, S128A, N273Q, N345Q, N384Q, N448Q, N496Q. In some embodiments, the G/H glycoprotein is a mutant NiV-G containing two amino acid substitutions from any two of N39Q, N126Q, S128A, N273Q, N345Q, N384Q, N448Q, N496Q. In some embodiments, the G/H glycoprotein is a mutant NiV-G containing three amino acid substitutions from any three of N39Q, N126Q, S128A, N273Q, N345Q, N384Q, N448Q, N496Q. In some embodiments, the G/H glycoprotein is a mutant NiV-G containing four amino acid substitutions from any one of N39Q, N126Q, S128A, N273Q, N345Q, N384Q, N448Q, N496Q. In some embodiments, the G/H glycoprotein is a mutant NiV-G containing five amino acid substitutions from any one of N39Q, N126Q, S128A, N273Q, N345Q, N384Q, N448Q, N496Q. In some embodiments, the G/H glycoprotein is a mutant NiV-G containing six amino acid substitutions from any one of N39Q, N126Q, S128A, N273Q, N345Q, N384Q, N448Q, N496Q. In some embodiments, the G/H glycoprotein is a mutant NiV-G containing seven amino acid substitutions from any one of N39Q, N126Q, S128A, N273Q, N345Q, N384Q, N448Q, N496Q. In some embodiments, the G/H glycoprotein is a mutant NiV-G containing eight amino acid substitutions from any one of N39Q, N126Q, S128A, N273Q, N345Q, N384Q, N448Q, N496Q. In some embodiments, the one or more amino acid substitutions are in the SEQ ID NO:147 or a or a modified Nipah G glycoprotein (NiV-G) that has an altered cytoplasmic tail compared to native NiV-G (e.g., SEQ ID NO:42). In some embodiments, the amino acid substitutions are in a modified NiV-G protein described in Section II.B. In some embodiments, the amino acid substitutions are in the NiV-G set forth in SEQ ID NO:42.

In some embodiments, the variant Nipah-G protein comprises at least three amino acid substitutions. In some embodiments, the amino acid substitutions are at positions 273, 384, and 496 of SEQ ID NO:42. In some embodiments, the amino acid substitutions are at positions 273, 345, and 496 of SEQ ID NO:42. In some embodiments, the amino acid substitutions are at positions 39, 126, and 128 of SEQ ID NO:42. In some embodiments, the amino acid substitutions are at positions 39, 273, and 345 of SEQ ID NO:42. In some embodiments, the amino acid substitutions are at positions 39, 384, and 448 of SEQ ID NO:42. In some embodiments, the amino acid substitutions are at positions 39, 448, and 496 of SEQ ID NO:42. In some embodiments, the amino acid substitutions are at positions 39, 128, and 273 of SEQ ID NO:42. In some embodiments, the amino acid substitutions are at positions 39, 345, and 384 of SEQ ID NO:42. In some embodiments, the amino acid substitutions are at positions 39, 384, and 448 of SEQ ID NO:42.

In some embodiments, the variant Nipah-G protein comprises at least two amino acid substitutions. In some embodiments, the amino acid substitutions are at positions 273, and 496 of SEQ ID NO:42. In some embodiments, the amino acid substitutions are at positions 345, and 496 of SEQ ID NO:42. In some embodiments, the amino acid substitutions are at positions 39 and 128 of SEQ ID NO:42. In some embodiments, the amino acid substitutions are at positions 39, and 345 of SEQ ID NO:42. In some embodiments, the amino acid substitutions are at positions 39, and 448 of SEQ ID NO:42. In some embodiments, the amino acid substitutions are at positions 39 and 496 of SEQ ID NO:42. In some embodiments, the amino acid substitutions are at positions 39 and 273 of SEQ ID NO:42. In some embodiments, the amino acid substitutions are at positions 39 and 384 of SEQ ID NO:42. In some embodiments, the amino acid substitutions are at positions 384 and 448 of SEQ ID NO:42.

In some embodiments, the amino acid substitution is at position 39 of SEQ ID NO:42. In some embodiments, the amino acid substitution is at position 126 of SEQ ID NO:42. In some embodiments, the amino acid substitution is at position 128 of SEQ ID NO:42. In some embodiments, the amino acid substitution is at position 273 of SEQ ID NO:42. In some embodiments, the amino acid substitution is at position 345 of SEQ ID NO:42. In some embodiments, the amino acid substitution is at position 384 of SEQ ID NO:42. In some embodiments, the amino acid substitution is at position 448 of SEQ ID NO:42. In some embodiments, the amino acid substitution is at position 496 of SEQ ID NO:42.

In some embodiments, the mutant Nipah-G protein comprises an asparagine to glutamine substitution at position 39 of SEQ ID NO:42. In some embodiments, the mutant Nipah-G protein comprises an asparagine to glutamine substitution at position 126 of SEQ ID NO:42. In some embodiments, the mutant Nipah-G protein comprises an asparagine to glutamine substitution at position 273 of SEQ ID NO:42. In some embodiments, the mutant Nipah-G protein comprises an asparagine to glutamine substitution at position 345 of SEQ ID NO:42. In some embodiments, the mutant Nipah-G protein comprises an asparagine to glutamine substitution at position 384 of SEQ ID NO:42. In some embodiments, the mutant Nipah-G protein comprises an asparagine to glutamine substitution at position 448 of SEQ ID NO:42. In some embodiments, the mutant Nipah-G protein comprises an asparagine to glutamine substitution at position 496 of SEQ ID NO:42. In some embodiments, the mutant Nipah-G protein comprises a serine to alanine substitution at position 128 of SEQ ID NO:42.

In some embodiments, the mutant Nipah-G protein comprises the sequence selected from the group consisting of any one of SEQ ID NOs: 377-503, In some embodiments, the mutant Nipah-G protein comprises the sequence of SEQ ID NO: 379. In some embodiments, the variant Nipah-G protein comprises the sequence of SEQ ID NO: 400. In some embodiments, the variant Nipah-G protein comprises the sequence of SEQ ID NO: 404.

In some embodiments, the Paramyxovirus G/H glycoprotein to which the deglycosylation mutations is made is a Measles virus H (Mev-H) protein or a modified MeV-H protein that has an altered cytoplasmic tail compared to native MeV-H (e.g., SEQ ID NO:375). In some embodiments, the mutant Paramyxovirus G/H protein has a sequence of amino acids that has at least 85%, at least 86%, at least 87%, at least 88%, at least 89%, at least 90%, at least 91%, at least 92%, at least 93%, at least 94% or at least 95% to SEQ ID NO: 375 and contains the acid substitution at one or more amino acid positions that reduce glycosylation of the G/H glycoprotein as provided herein.

In some embodiments, the G/H glycoprotein is derived from Measles virus H (Mev-H) protein and the one or more amino acid substitutions are at positions corresponding to positions selected from the group consisting of 168, 187, 200, 215, 238 of SEQ ID NO: 375. In some embodiments, the mutant Mev-H protein comprises at least two amino acid substitutions, such as 2, 3, 4, or 5 substitutions at positions 168, 187, 200, 215, 238 of SEQ ID NO: 375.

In some embodiments, the Paramyxovirus G/H glycoprotein to which the deglycosylation mutations is made is a Canine distemper virus H (CDV-H) protein or a modified CDV-H protein that has an altered cytoplasmic tail compared to native CDV-H (e.g., SEQ ID NO:376). In some embodiments, the mutant Paramyxovirus G/H protein has a sequence of amino acids that has at least 85%, at least 86%, at least 87%, at least 88%, at least 89%, at least 90%, at least 91%, at least 92%, at least 93%, at least 94% or at least 95% to SEQ ID NO: 376 and contains the acid substitution at one or more amino acid positions that reduce glycosylation of the G/H glycoprotein as provided herein.

In some embodiments, the G/H glycoprotein is derived from Canine distemper virus H (CDV-H) protein and the one or more amino acid substitutions are at positions corresponding to positions selected from the group consisting of 19, 149, 422 of SEQ ID NO: 376. In some embodiments, the variant CDV-H protein comprises at least two amino acid substitutions, such as 2 or 3 substitutions at positions 19, 149, 422 of SEQ ID NO: 376.

In some embodiments, the G protein is a mutant G protein that is a biologically active portion that is an N-terminally and/or C-terminally truncated fragment of a wild-type Hendra (HeV) virus G protein, a wild-type Nipah (NiV) virus G-protein (NiV-G), a wild-type Cedar (CedPV) virus G-protein, a wild-type Mojiang virus G-protein, a wild-type bat Paramyxovirus G-protein, a Kumasi virus G-protein, or a Langya virus G-protein. In particular embodiments, the truncation is an N-terminal truncation of all or a portion of the cytoplasmic domain. In some embodiments, the mutant G protein is a biologically active portion that is truncated and lacks up to 49 contiguous amino acid residues at or near the N-terminus of the wild-type G protein, such as a wild-type G protein set forth in any one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO: 10 or SEQ ID NO:11. In some embodiments, the mutant F protein is truncated and lacks up to 49 contiguous amino acids, such as up to 49, 48, 47, 46, 45, 44, 43, 42, 41, 40, 30, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 contiguous amino acids at the N-terminus of the wild-type G protein.

In some embodiments, the G protein is a wild-type Nipah virus G (NiV-G) protein or a Hendra virus G protein, or is a functionally active variant or biologically active portion thereof. In some embodiments, the G protein is a NiV-G protein that has the sequence set forth in SEQ ID NO:1, SEQ ID NO:4 or SEQ ID NO:5, or is a functional variant or a biologically active portion thereof that has an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, at least at or about 99% sequence identity to SEQ ID NO:1, SEQ ID NO:4 or SEQ ID NO:5. In some embodiments, the G protein is a NiV-G protein that has the sequence set forth in SEQ ID NO:1, or is a functional variant or a biologically active portion thereof that has an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, at least at or about 99% sequence identity to SEQ ID NO:1. In some embodiments, the G protein is a NiV-G protein that has the sequence set forth in SEQ ID NO:1. In some embodiments, the G protein is a NiV-G protein that has the sequence set forth in SEQ ID NO:4, or is a functional variant or a biologically active portion thereof that has an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, at least at or about 99% sequence identity to SEQ ID NO:4. In some embodiments, the G protein is a NiV-G protein that has the sequence set forth in SEQ ID NO:4. In some embodiments, the G protein is a NiV-G protein that has the sequence set forth in SEQ ID NO:5, or is a functional variant or a biologically active portion thereof that has an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, at least at or about 99% sequence identity to SEQ ID NO:5. In some embodiments, the G protein is a NiV-G protein that has the sequence set forth in SEQ ID NO:5.

In some embodiments, the G protein is a mutant NiV-G protein that is a biologically active portion of a wild-type NiV-G. In some embodiments, the biologically active portion is an N-terminally truncated fragment. In some embodiments, the mutant NiV-G protein is truncated and lacks up to 5 contiguous amino acid residues at or near the N-terminus of the wild-type NiV-G protein (SEQ ID NO:1, SEQ ID NO:4 or SEQ ID NO:5), up to 6 contiguous amino acid residues at or near the N-terminus of the wild-type NiV-G protein (SEQ ID NO:1, SEQ ID NO:4 or SEQ ID NO:5), up to 7 contiguous amino acid residues at or near the N-terminus of the wild-type NiV-G protein (SEQ ID NO:1, SEQ ID NO:4 or SEQ ID NO:5), up to 8 contiguous amino acid residues at or near the N-terminus of the wild-type NiV-G protein (SEQ ID NO:1, SEQ ID NO:4 or SEQ ID NO:5), up to 9 contiguous amino acid residues at or near the N-terminus of the wild-type NiV-G protein (SEQ ID NO:1, SEQ ID NO:4 or SEQ ID NO:5) up to 10 contiguous amino acid residues at or near the N-terminus of the wild-type NiV-G protein (SEQ ID NO:1, SEQ ID NO:4 or SEQ ID NO:5), up to 11 contiguous amino acid residues at or near the N-terminus of the wild-type NiV-G protein (SEQ ID NO:1, SEQ ID NO:4 or SEQ ID NO:5), up to 12 contiguous amino acid residues at or near the N-terminus of the wild-type NiV-G protein SEQ ID NO:1, SEQ ID NO:4, or SEQ ID NO:5), up to 13 contiguous amino acid residues at or near the N-terminus of the wild-type NiV-G protein (SEQ ID NO:1, SEQ ID NO:4 or SEQ ID NO:5), up to 14 contiguous amino acid residues at or near the N-terminus of the wild-type NiV-G protein (SEQ ID NO:1, SEQ ID NO:4 or SEQ ID NO:5), up to 15 contiguous amino acid residues at or near the N-terminus of the wild-type NiV-G protein (SEQ ID NO:1, SEQ ID NO:4 or SEQ ID NO:5) up to 16 contiguous amino acid residues at or near the N-terminus of the wild-type NiV-G protein (SEQ ID NO:1, SEQ ID NO:4 or SEQ ID NO:5), up to 17 contiguous amino acid residues at or near the N-terminus of the wild-type NiV-G protein (SEQ ID NO:1, SEQ ID NO:4 or SEQ ID NO:5), up to 18 contiguous amino acid residues at or near the N-terminus of the wild-type NiV-G protein (SEQ ID NO:1, SEQ ID NO:4 or SEQ ID NO:5), up to 19 contiguous amino acid residues at or near the N-terminus of the wild-type NiV-G protein (SEQ ID NO:1, SEQ ID NO:4 or SEQ ID NO:5), up to 20 contiguous amino acid residues at or near the N-terminus of the wild-type NiV-G protein (SEQ ID NO:1, SEQ ID NO:4 or SEQ ID NO:5), up to 21 contiguous amino acid residues at or near the N-terminus of the wild-type NiV-G protein SEQ ID NO:1, SEQ ID NO:4 or SEQ ID NO:5), up to 22 contiguous amino acid residues at or near the N-terminus of the wild-type NiV-G protein (SEQ ID NO:1, SEQ ID NO:4 or SEQ ID NO:5), up to 23 contiguous amino acid residues at or near the N-terminus of the wild-type NiV-G protein (SEQ ID NO:1, SEQ ID NO:4 or SEQ ID NO:5), up to 24 contiguous amino acid residues at or near the N-terminus of the wild-type NiV-G protein (SEQ ID NO:1, SEQ ID NO:4 or SEQ ID NO:5), up to 25 contiguous amino acid residues at or near the N-terminus of the wild-type NiV-G protein (SEQ ID NO:1, SEQ ID NO:4 or SEQ ID NO:5), up to 26 contiguous amino acid residues at or near the N-terminus of the wild-type NiV-G protein (SEQ ID NO:1, SEQ ID NO:4 or SEQ ID NO:5), up to 27 contiguous amino acid residues at or near the N-terminus of the wild-type NiV-G protein (SEQ ID NO:1, SEQ ID NO:4 or SEQ ID NO:5), up to 28 contiguous amino acid residues at or near the N-terminus of the wild-type NiV-G protein (SEQ ID NO:1, SEQ ID NO:4 or SEQ ID NO:5), up to 29 contiguous amino acid residues at or near the N-terminus of the wild-type NiV-G protein (SEQ ID NO:1, SEQ ID NO:4 or SEQ ID NO:5), up to 30 contiguous amino acid residues at or near the N-terminus of the wild-type NiV-G protein (SEQ ID NO:1, SEQ ID NO:4 or SEQ ID NO:5), up to 31 contiguous amino acid residues at or near the N-terminus of the wild-type NiV-G protein (SEQ ID NO:1, SEQ ID NO:4 or SEQ ID NO:5), up to 32 contiguous amino acid residues at or near the N-terminus of the wild-type NiV-G protein (SEQ ID NO:1, SEQ ID NO:4 or SEQ ID NO:5), up to 33 contiguous amino acid residues at or near the N-terminus of the wild-type NiV-G protein (SEQ ID NO:1, SEQ ID NO:4 or SEQ ID NO:5), up to 34 contiguous amino acid residues at or near the N-terminus of the wild-type NiV-G protein (SEQ ID NO:1, SEQ ID NO:4 or SEQ ID NO:5), up to 35 contiguous amino acid residues at or near the N-terminus of the wild-type NiV-G protein (SEQ ID NO:1, SEQ ID NO:4 or SEQ ID NO:5), up to 36 contiguous amino acid residues at or near the N-terminus of the wild-type NiV-G protein (SEQ ID NO:1, SEQ ID NO:4 or SEQ ID NO:5), up to 37 contiguous amino acid residues at or near the N-terminus of the wild-type NiV-G protein (SEQ ID NO:1, SEQ ID NO:4 or SEQ ID NO:5) up to 38 contiguous amino acid residues at or near the N-terminus of the wild-type NiV-G protein (SEQ ID NO:1, SEQ ID NO:4 or SEQ ID NO:5), up to 39 contiguous amino acid residues at or near the N-terminus of the wild-type NiV-G protein (SEQ ID NO:1, SEQ ID NO:4 or SEQ ID NO:5), up to 40 contiguous amino acid residues at or near the N-terminus of the wild-type NiV-G protein (SEQ ID NO:1, SEQ ID NO:4 or SEQ ID NO:5), up to 41 contiguous amino acid residues at or near the N-terminus of the wild-type NiV-G protein (SEQ ID NO:1, SEQ ID NO:4 or SEQ ID NO:5), up to 42 contiguous amino acid residues at or near the N-terminus of the wild-type NiV-G protein (SEQ ID NO:1, SEQ ID NO:4 or SEQ ID NO:5), up to 43 contiguous amino acid residues at or near the N-terminus of the wild-type NiV-G protein (SEQ ID NO:1, SEQ ID NO:4 or SEQ ID NO:5), up to 44 contiguous amino acid residues at or near the N-terminus of the wild-type NiV-G protein (SEQ ID NO:1, SEQ ID NO:4 or SEQ ID NO:5), or up to 45 contiguous amino acid residues at or near the N-terminus of the wild-type NiV-G protein (SEQ ID NO:1, SEQ ID NO:4 or SEQ ID NO:5).

In some embodiments, the mutant NiV-G protein is truncated and lacks 5 contiguous amino acid residues at or near the N-terminus of the wild-type NiV-G protein (SEQ ID NO:1, SEQ ID NO:4 or SEQ ID NO:5). In some embodiments, the mutant NiV-G protein comprises the amino acid sequence set forth in SEQ ID NO:12. In some embodiments, the mutant NiV-G protein is truncated and lacks 10 contiguous amino acid residues at or near the N-terminus of the wild-type NiV-G protein (SEQ ID NO:1, SEQ ID NO:4 or SEQ ID NO:5). In some embodiments, the mutant NiV-G protein comprises the amino acid sequence set forth in SEQ ID NO:44. In some embodiments, the mutant NiV-G protein is truncated and lacks 15 contiguous amino acid residues at or near the N-terminus of the wild-type NiV-G protein (SEQ ID NO:1, SEQ ID NO:4 or SEQ ID NO:5). In some embodiments, the mutant NiV-G protein comprises the amino acid sequence set forth in SEQ ID NO:45. In some embodiments, the mutant NiV-G protein is truncated and lacks 20 contiguous amino acid residues at or near the N-terminus of the wild-type NiV-G protein (SEQ ID NO:1, SEQ ID NO:4 or SEQ ID NO:5). In some embodiments, the mutant NiV-G protein comprises the amino acid sequence set forth in SEQ ID NO:13. In some embodiments, the mutant NiV-G protein is truncated and lacks 25 contiguous amino acid residues at or near the N-terminus of the wild-type NiV-G protein (SEQ ID NO:1, SEQ ID NO:4 or SEQ ID NO:5). In some embodiments, the mutant NiV-G protein comprises the amino acid sequence set forth in SEQ ID NO:14. In some embodiments, the mutant NiV-G protein is truncated and lacks 30 contiguous amino acid residues at or near the N-terminus of the wild-type NiV-G protein (SEQ ID NO:1, SEQ ID NO:4 or SEQ ID NO:5). In some embodiments, the mutant NiV-G protein comprises the amino acid sequence set forth in SEQ ID NO:43. In some embodiments, the mutant NiV-G protein is truncated and lacks 34 contiguous amino acid residues at or near the N-terminus of the wild-type NiV-G protein (SEQ ID NO:1, SEQ ID NO:4 or SEQ ID NO:5). In some embodiments, the mutant NiV-G protein comprises the amino acid sequence set forth in SEQ ID NO:42.

In some embodiments, the NiV-G protein is a biologically active portion that does not contain a cytoplasmic domain. In some embodiments, the NiV-G protein without the cytoplasmic domain is encoded by SEQ ID NO:22.

In some embodiments, the mutant NiV-G protein comprises a sequence set forth in any of SEQ ID NOS: 12-14, 17, 18 and 22, or 42-45 or is a functional variant thereof that has an amino acid sequence having at least at or 80%, at least at or about 81%, at least at or about 82%, at least at or about 83%, at or about 84%, at least at or about 85%, at least at or about 86%, or at least at or about 87%, at least at or about 88%, or at least at or about 89%, about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NOS: 12-14, 17, 18 and 22 or 42-45.

In some embodiments, the mutant NiV-G protein has a 5 amino acid truncation at or near the N-terminus of the wild-type NiV-G protein (SEQ ID NO:1, SEQ ID NO:4 or SEQ ID NO:5), such as set forth in SEQ ID NO:12 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:12 or such as set forth in SEQ ID NO:17 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:17. In some embodiments, the mutant NiV-G protein has a 10 amino acid truncation at or near the N-terminus of the wild-type NiV-G protein (SEQ ID NO:1, SEQ ID NO:4 or SEQ ID NO:5), such as set forth in SEQ ID NO:44 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:44. In some embodiments, the mutant NiV-G protein has a 20 amino acid truncation at or near the N-terminus of the wild-type NiV-G protein (SEQ ID NO:1, SEQ ID NO:4 or SEQ ID NO:5), such as set forth in SEQ ID NO:13 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:13. In some embodiments, the mutant NiV-G protein has a 25 amino acid truncation at or near the N-terminus of the wild-type NiV-G protein (SEQ ID NO:1, SEQ ID NO:4 or SEQ ID NO:5), such as set forth in SEQ ID NO:14 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:14. In some embodiments, the mutant NiV-G protein has a 33 amino acid truncation at or near the N-terminus of the wild-type NiV-G protein (SEQ ID NO:1, SEQ ID NO:4 or SEQ ID NO:5), such as set forth in SEQ ID NO:17 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:17. In some embodiments, the mutant NiV-G protein has a 34 amino acid truncation at or near the N-terminus of the wild-type NiV-G protein (SEQ ID NO:1, SEQ ID NO:4 or SEQ ID NO:5), such as set forth in SEQ ID NO:18 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:18. In some embodiments, the mutant NiV-G protein has a 48 amino acid truncation at or near the N-terminus of the wild-type NiV-G protein (SEQ ID NO:1, SEQ ID NO:4 or SEQ ID NO:5), such as set forth in SEQ ID NO:22 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:22.

In some embodiments, the mutant NiV-G protein has a 15 amino acid truncation at or near the N-terminus of the wild-type NiV-G protein (SEQ ID NO:1, SEQ ID NO:4, or SEQ ID NO:5), such as set forth in SEQ ID NO:45 or a functional variant thereof that has an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:45.

In some embodiments, the mutant NiV-G protein has a 20 amino acid truncation at or near the N-terminus of the wild-type NiV-G protein (SEQ ID NO:1, SEQ ID NO:4, or SEQ ID NO:5), such as set forth in SEQ ID NO:13 or a functional variant thereof that has an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:13.

In some embodiments, the mutant NiV-G protein has a 25 amino acid truncation at or near the N-terminus of the wild-type NiV-G protein (SEQ ID NO:1, SEQ ID NO:4, or SEQ ID NO:5), such as set forth in SEQ ID NO:14 or a functional variant thereof that has an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:14.

In some embodiments, the mutant NiV-G protein has a 30 amino acid truncation at or near the N-terminus of the wild-type NiV-G protein (SEQ ID NO:1, SEQ ID NO:4, or SEQ ID NO:5), such as set forth in SEQ ID NO:43 or a functional variant thereof that has an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:43.

In some embodiments, the mutant NiV-G protein has a 34 amino acid truncation at or near the N-terminus of the wild-type NiV-G protein (SEQ ID NO:1, SEQ ID NO:4, or SEQ ID NO:5), such as set forth in SEQ ID NO:42 or a functional variant thereof that has an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:42.

In some embodiments, the mutant NiV-G protein has a 48 amino acid truncation at or near the N-terminus of the wild-type NiV-G protein (SEQ ID NO:1, SEQ ID NO:4, or SEQ ID NO:5), such as set forth in SEQ ID NO:22 or a functional variant thereof that has an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:22.

In some embodiments, the G protein is a mutant HeV-G protein that is a biologically active portion of a wild-type HeV-G. In some embodiments, the biologically active portion is an N-terminally truncated fragment.

In some embodiments, the G protein is a wild-type HeV-G protein that has the sequence set forth in SEQ ID NO:23 or 24, or is a functional variant or biologically active portion thereof that has an amino acid sequence having at least at or about 80%, at least at or about 81%, at least at or about 82%, at least at or about 83%, at least at or about 84%, at or about 85%, at least at or about 86%, at least at or about 87%, at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:23 or 24.

In some embodiments, the G protein is a mutant HeV-G protein that is a biologically active portion of a wild-type HeV-G (SEQ ID NO:23 or SEQ ID NO:24). In some embodiments, the biologically active portion is an N-terminally truncated fragment. In some embodiments, the mutant HeV-G protein is truncated and lacks up to 5 contiguous amino acid residues at or near the N-terminus of the wild-type HeV-G protein (SEQ ID NO:23 or 24), up to 6 contiguous amino acid residues at or near the N-terminus of the wild-type HeV-G protein (SEQ ID NO:23 or 24), up to 7 contiguous amino acid residues at or near the N-terminus of the wild-type HeV-G protein (SEQ ID NO:23 or 24) or up to 8 contiguous amino acid residues at or near the N-terminus of the wild-type HeV-G protein (SEQ ID NO:23 or 24), up to 9 contiguous amino acid residues at or near the N-terminus of the wild-type HeV-G protein (SEQ ID NO:23 or 24), up to 10 contiguous amino acid residues at or near the N-terminus of the wild-type HeV-G protein (SEQ ID NO:23 or 24), up to 11 contiguous amino acid residues at or near the N-terminus of the wild-type HeV-G protein (SEQ ID NO:23 or 24), up to 12 contiguous amino acid residues at or near the N-terminus of the wild-type HeV-G protein (SEQ ID NO:23 or 24), up to 13 contiguous amino acid residues at or near the N-terminus of the wild-type HeV-G protein (SEQ ID NO:23 or 24), up to 14 contiguous amino acid residues at or near the N-terminus of the wild-type HeV-G protein (SEQ ID NO:23 or 24), up to 15 contiguous amino acid residues at or near the N-terminus of the wild-type HeV-G protein (SEQ ID NO:23 or 24), up to 16 contiguous amino acid residues at or near the N-terminus of the wild-type HeV-G protein (SEQ ID NO:23 or 24), up to 17 contiguous amino acid residues at or near the N-terminus of the wild-type HeV-G protein (SEQ ID NO:23 or 24), up to 18 contiguous amino acid residues at or near the N-terminus of the wild-type HeV-G protein (SEQ ID NO:23 or 24), up to 19 contiguous amino acid residues at or near the N-terminus of the wild-type HeV-G protein (SEQ ID NO:23 or 24), up to 20 contiguous amino acid residues at or near the N-terminus of the wild-type HeV-G protein (SEQ ID NO:23 or 24), up to 21 contiguous amino acid residues at or near the N-terminus of the wild-type HeV-G protein (SEQ ID NO:23 or 24), up to 22 contiguous amino acid residues at or near the N-terminus of the wild-type HeV-G protein (SEQ ID NO:23 or 24), up to 23 contiguous amino acid residues at or near the N-terminus of the wild-type HeV-G protein SEQ ID NO:23 or 24), up to 24 contiguous amino acid residues at or near the N-terminus of the wild-type HeV-G protein (SEQ ID NO:23 or 24), up to 25 contiguous amino acid residues at or near the N-terminus of the wild-type HeV-G protein (SEQ ID NO:23 or 24), up to 26 contiguous amino acid residues at or near the N-terminus of the wild-type HeV-G protein (SEQ ID NO:23 or 24), up to 27 contiguous amino acid residues at or near the N-terminus of the wild-type HeV-G protein (SEQ ID NO:23 or 24), up to 28 contiguous amino acid residues at or near the N-terminus of the wild-type HeV-G protein (SEQ ID NO:23 or 24), up to 29 contiguous amino acid residues at or near the N-terminus of the wild-type HeV-G protein (SEQ ID NO:23 or 24), up to 30 contiguous amino acid residues at or near the N-terminus of the wild-type HeV-G protein (SEQ ID NO:23 or 24), up to 31 contiguous amino acid residues at or near the N-terminus of the wild-type HeV-G protein (SEQ ID NO:23 or 24), up to 32 contiguous amino acid residues at or near the N-terminus of the wild-type HeV-G protein (SEQ ID NO:23 or 24), up to 33 contiguous amino acid residues at or near the N-terminus of the wild-type HeV-G protein (SEQ ID NO:23 or 24), up to 34 contiguous amino acid residues at or near the N-terminus of the wild-type HeV-G protein (SEQ ID NO:23 or 24), up to 35 contiguous amino acid residues at or near the N-terminus of the wild-type HeV-G protein (SEQ ID NO:23 or 24), up to 36 contiguous amino acid residues at or near the N-terminus of the wild-type HeV-G protein (SEQ ID NO:23 or 24), up to 37 contiguous amino acid residues at or near the N-terminus of the wild-type HeV-G protein (SEQ ID NO:23 or 24), up to 38 contiguous amino acid residues at or near the N-terminus of the wild-type HeV-G protein (SEQ ID NO:23 or 24), up to 39 contiguous amino acid residues at or near the N-terminus of the wild-type HeV-G protein (SEQ ID NO:23 or 24), up to 40 contiguous amino acid residues at or near the N-terminus of the wild-type HeV-G protein (SEQ ID NO:23 or 24), up to 41 contiguous amino acid residues at or near the N-terminus of the wild-type HeV-G protein (SEQ ID NO:23 or 24), up to 42 contiguous amino acid residues at or near the N-terminus of the wild-type HeV-G protein (SEQ ID NO:23 or 24), up to 43 contiguous amino acid residues at or near the N-terminus of the wild-type HeV-G protein (SEQ ID NO:23 or 24), up to 44 contiguous amino acid residues at or near the N-terminus of the wild-type HeV-G protein (SEQ ID NO:23 or 24), or up to 45 contiguous amino acid residues at or near the N-terminus of the wild-type HeV-G protein (SEQ ID NO:23 or 24).

In some embodiments, the HeV-G protein is a biologically active portion that does not contain a cytoplasmic domain. In some embodiments, the mutant HeV-G protein lacks the N-terminal cytoplasmic domain of the wild-type HeV-G protein (SEQ ID NO:23 or 24), such as set forth in SEQ ID NO:25 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:25. In some embodiments, the mutant HeV-G protein lacks the N-terminal cytoplasmic domain of the wild-type HeV-G protein (SEQ ID NO:23 or 24), such as set forth in SEQ ID NO:26 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:26.

In some embodiments, the G protein or the functionally active variant or biologically active portion thereof binds to Ephrin B2 or Ephrin B3. In some aspects, the G protein has the sequence of amino acids set forth in any one of SEQ ID NO:24, SEQ ID NO:23, SEQ ID NO:4, SEQ ID NO:6, SEQ ID NO:5, SEQ ID NO:8 or SEQ ID NO:10, or is a functionally active variant thereof or a biologically active portion thereof that is able to bind to Ephrin B2 or Ephrin B3. In some embodiments, the functionally active variant or biologically active portion has an amino acid sequence having at least at or about 80%, at least at or about 81%, at least at or about 82%, at least at or about 83%, at least at or about 84%, at least at or about 85%, at or about 86%, at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to any of SEQ ID NO:24, SEQ ID NO:23, SEQ ID NO:4, SEQ ID NO:6, SEQ ID NO:5, SEQ ID NO:8 or SEQ ID NO:10, or a functionally active variant or biologically active portion thereof, and retains binding to Ephrin B2 or B3.

In some embodiments, the functionally active variant or biologically active portion has an amino acid sequence having at least about 80%, at least about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:27, SEQ ID NO:23, SEQ ID NO:4, SEQ ID NO:6, SEQ ID NO:5, SEQ ID NO:8 or SEQ ID NO: 10, or a functionally active variant or biologically active portion thereof, and retains binding to Ephrin B2 or B3. Reference to retaining binding to Ephrin B2 or B3 includes binding that is at least or at least about 5% of the level or degree of binding of the corresponding wild-type G protein, such as set forth in SEQ ID NO:27, SEQ ID NO:23, SEQ ID NO:4, SEQ ID NO:6, SEQ ID NO:5, SEQ ID NO:8 or SEQ ID NO:10, or a functionally active variant or biologically active portion thereof, 10% of the level or degree of binding of the corresponding wild-type G protein, such as set forth in SEQ ID NO:27, SEQ ID NO:23, SEQ ID NO:4, SEQ ID NO:6, SEQ ID NO:5, SEQ ID NO:8 or SEQ ID NO:10, or a functionally active variant or biologically active portion thereof, 15% of the level or degree of binding of the corresponding wild-type G protein, such as set forth in SEQ ID NO:27, SEQ ID NO:23, SEQ ID NO:4, SEQ ID NO:6, SEQ ID NO:5, SEQ ID NO:8 or SEQ ID NO:10, or a functionally active variant or biologically active portion thereof, 20% of the level or degree of binding of the corresponding wild-type G protein, such as set forth in SEQ ID NO:27, SEQ ID NO:23, SEQ ID NO:4, SEQ ID NO:6, SEQ ID NO:5, SEQ ID NO:8 or SEQ ID NO:10, or a functionally active variant or biologically active portion thereof, 25% of the level or degree of binding of the corresponding wild-type G protein, such as set forth in SEQ ID NO:27, SEQ ID NO:23, SEQ ID NO:4, SEQ ID NO:6, SEQ ID NO:5, SEQ ID NO:8 or SEQ ID NO:10, or a functionally active variant or biologically active portion, 30% of the level or degree of binding of the corresponding wild-type G protein, such as set forth in SEQ ID NO:27, SEQ ID NO:23, SEQ ID NO:4, SEQ ID NO:6, SEQ ID NO:5, SEQ ID NO:8 or SEQ ID NO:10, or a functionally active variant or biologically active portion thereof, 35% of the level or degree of binding of the corresponding wild-type G protein, such as set forth in SEQ ID NO:27, SEQ ID NO:23, SEQ ID NO:4, SEQ ID NO:6, SEQ ID NO:5, SEQ ID NO:8 or SEQ ID NO:10, or a functionally active variant or biologically active portion thereof, 40% of the level or degree of binding of the corresponding wild-type G protein, such as set forth in SEQ ID NO:27, SEQ ID NO:23, SEQ ID NO:4, SEQ ID NO:6, SEQ ID NO:5, SEQ ID NO:8 or SEQ ID NO:10, or a functionally active variant or biologically active portion thereof, 45% of the level or degree of binding of the corresponding wild-type G protein, such as set forth in SEQ ID NO:27, SEQ ID NO:23, SEQ ID NO:4, SEQ ID NO:6, SEQ ID NO:5, SEQ ID NO:8 or SEQ ID NO:10, or a functionally active variant or biologically active portion thereof, 50% of the level or degree of binding of the corresponding wild-type G protein, such as set forth in SEQ ID NO:27, SEQ ID NO:23, SEQ ID NO:4, SEQ ID NO:6, SEQ ID NO:5, SEQ ID NO:8 or SEQ ID NO:10, or a functionally active variant or biologically active portion thereof, 55% of the level or degree of binding of the corresponding wild-type G protein, such as set forth in SEQ ID NO:27, SEQ ID NO:23, SEQ ID NO:4, SEQ ID NO:6, SEQ ID NO:5, SEQ ID NO:8 or SEQ ID NO:10, or a functionally active variant or biologically active portion thereof, 60% of the level or degree of binding of the corresponding wild-type G protein, such as set forth in SEQ ID NO:27, SEQ ID NO:23, SEQ ID NO:4, SEQ ID NO:6, SEQ ID NO:5, SEQ ID NO:8 or SEQ ID NO:10, or a functionally active variant or biologically active portion thereof, 65% of the level or degree of binding of the corresponding wild-type G protein, such as set forth in SEQ ID NO:27, SEQ ID NO:23, SEQ ID NO:4, SEQ ID NO:6, SEQ ID NO:5, SEQ ID NO:8 or SEQ ID NO:10, or a functionally active variant or biologically active portion thereof, 70% of the level or degree of binding of the corresponding wild-type G protein, such as set forth in SEQ ID NO:27, SEQ ID NO:23, SEQ ID NO:4, SEQ ID NO:6, SEQ ID NO:5, SEQ ID NO:8 or SEQ ID NO:10 or a functionally active variant or biologically active portion thereof, such as at least or at least about 75% of the level or degree of binding of the corresponding wild-type G protein, such as set forth in SEQ ID NO:27, SEQ ID NO:23, SEQ ID NO:4, SEQ ID NO:6, SEQ ID NO:5, SEQ ID NO:8 or SEQ ID NO:10, or a functionally active variant or biologically active portion thereof, such as at least or at least about 80% of the level or degree of binding of the corresponding wild-type G protein, such as set forth in SEQ ID NO:27, SEQ ID NO:23, SEQ ID NO:4, NO:4,SEQ ID NO:6, SEQ ID NO:5, SEQ ID NO:8 or SEQ ID NO:10, or a functionally active variant or biologically active portion thereof, such as at least or at least about 85% of the level or degree of binding of the corresponding wild-type G protein, such as set forth in SEQ ID NO:27, SEQ ID NO:23, SEQ ID NO:4,SEQ ID NO:6, SEQ ID NO:5, SEQ ID NO:8 or SEQ ID NO:10, or a functionally active variant or biologically active portion thereof, such as at least or at least about 90% of the level or degree of binding of the corresponding wild-type G protein, such as set forth in SEQ ID NO:27, SEQ ID NO:23, SEQ ID NO:4,SEQ ID NO:6, SEQ ID NO:5, SEQ ID NO:8 or SEQ ID NO:10, or a functionally active variant or biologically active portion thereof, or such as at least or at least about 95% of the level or degree of binding of the corresponding wild-type protein, such as set forth in SEQ ID NO:27, SEQ ID NO:23, SEQ ID NO:4,SEQ ID NO:6, SEQ ID NO:5, SEQ ID NO:8 or SEQ ID NO:10, or a functionally active variant or biologically active portion thereof. In some embodiments, the G protein is NiV-G or a functionally active variant or biologically active portion thereof and binds to Ephrin B2 or Ephrin B3. In some aspects, the NiV-G has the sequence of amino acids set forth in SEQ ID NO:4, SEQ ID NO:5 or SEQ ID NO:27, or is a functionally active variant thereof or a biologically active portion thereof that is able to bind to Ephrin B2 or Ephrin B3. In some embodiments, the functionally active variant or biologically active portion has an amino acid sequence having at least about 80%, at least about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:4, SEQ ID NO:5 or SEQ ID NO:27 and retains binding to Ephrin B2 or B3. Exemplary biologically active portions include N-terminally truncated variants lacking all or a portion of the cytoplasmic domain, e.g. 1 or more, such as 1 to 49 contiguous N-terminal amino acid residues. Reference to retaining binding to Ephrin B2 or B3 includes binding that is at least or at least about 5% of the level or degree of binding of the corresponding wild-type NiV-G, such as set forth in SEQ ID NO:4, SEQ ID NO:5 or SEQ ID NO:27, 10% of the level or degree of binding of the corresponding wild-type NiV-G, such as set forth in SEQ ID NO:4, SEQ ID NO:5 or SEQ ID NO:27, 15% of the level or degree of binding of the corresponding wild-type NiV-G, such as set forth in SEQ ID NO:4, SEQ ID NO:5 or SEQ ID NO:27, 20% of the level or degree of binding of the corresponding wild-type NiV-G, such as set forth in SEQ ID NO:4, SEQ ID NO:5 or SEQ ID NO:27, 25% of the level or degree of binding of the corresponding wild-type NiV-G, such as set forth in SEQ ID NO:4, SEQ ID NO:5 or SEQ ID NO:27, 30% of the level or degree of binding of the corresponding wild-type NiV-G, such as set forth in SEQ ID NO:4, SEQ ID NO:5 or SEQ ID NO:27, 35% of the level or degree of binding of the corresponding wild-type NiV-G, such as set forth in SEQ ID NO:4, SEQ ID NO:5 or SEQ ID NO:27, 40% of the level or degree of binding of the corresponding wild-type NiV-G, such as set forth in SEQ ID NO:4, SEQ ID NO:5 or SEQ ID NO:27, 45% of the level or degree of binding of the corresponding wild-type NiV-G, such as set forth in SEQ ID NO:4, SEQ ID NO:5 or SEQ ID NO:27 50% of the level or degree of binding of the corresponding wild-type NiV-G, such as set forth in SEQ ID NO:4, SEQ ID NO:5 or SEQ ID NO:27, 55% of the level or degree of binding of the corresponding wild-type NiV-G, such as set forth in SEQ ID NO:4, SEQ ID NO:5 or SEQ ID NO:27, 60% of the level or degree of binding of the corresponding wild-type NiV-G, such as set forth in SEQ ID NO:4, SEQ ID NO:5 or SEQ ID NO:27, 65% of the level or degree of binding of the corresponding wild-type NiV-G, such as set forth in SEQ ID NO:4, SEQ ID NO:5 or SEQ ID NO:27, 70% of the level or degree of binding of the corresponding wild-type NiV-G, such as set forth in SEQ ID NO:4, SEQ ID NO:5 or SEQ ID NO:27, such as at least or at least about 75% of the level or degree of binding of the corresponding wild-type NiV-G, such as set forth in SEQ ID NO:4, SEQ ID NO:5 or SEQ ID NO:27, such as at least or at least about 80% of the level or degree of binding of the corresponding wild-type NIV-G, such as set forth in SEQ ID NO:4, SEQ ID NO:5 or SEQ ID NO:27, such as at least or at least about 85% of the level or degree of binding of the corresponding wild-type NiV-G, such as set forth in SEQ ID NO:4, SEQ ID NO:5 or SEQ ID NO:27, such as at least or at least about 90% of the level or degree of binding of the corresponding wild-type NiV-G, such as set forth in SEQ ID NO:4, SEQ ID NO:5 or SEQ ID NO:27, or such as at least or at least about 95% of the level or degree of binding of the corresponding wild-type NiV-G, such as set forth in SEQ ID NO:4, SEQ ID NO:5 or SEQ ID NO:27.

In some embodiments, the G protein or the biologically thereof is a mutant G protein that exhibits reduced binding for the native binding partner of a wild-type G protein. In some embodiments, the mutant G protein or the biologically active portion thereof is a mutant of wild-type Niv-G and exhibits reduced binding to one or both of the native binding partners Ephrin B2 or Ephrin B3. In some embodiments, the mutant G-protein or the biologically active portion, such as a mutant NiV-G protein, exhibits reduced binding to the native binding partner. In some embodiments, the reduced binding to Ephrin B2 or Ephrin B3 is reduced by greater than at or about 5%, at or about 10%, at or about 15%, at or about 20%, at or about 25%, at or about 30%, at or about 40%, at or about 50%, at or about 60%, at or about 70%, at or about 80%, at or about 90%, or at or about 100%.

In some embodiments, the mutations described herein can improve transduction efficiency. In some embodiments, the mutations described herein allow for specific targeting of other desired cell types that are not Ephrin B2 or Ephrin B3. In some embodiments, the mutations described herein result in at least the partial inability to bind at least one natural receptor, such has reduced binding to at least one of Ephrin B2 or Ephrin B3. In some embodiments, the mutations described herein interfere with natural receptor recognition.

In some embodiments, the G protein is HeV-G or a functionally active variant or biologically active portion thereof and binds to Ephrin B2 or Ephrin B3. In some aspects, the HeV-G has the sequence of amino acids set forth in SEQ ID NO:23 or 24, or is a functionally active variant thereof or a biologically active portion thereof that is able to bind to Ephrin B2 or Ephrin B3. In some embodiments, the functionally active variant or biologically active portion has an amino acid sequence having at least about 80%, at least about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:23 or 24 and retains binding to Ephrin B2 or B3. Exemplary biologically active portions include N-terminally truncated variants lacking all or a portion of the cytoplasmic domain, e.g. 1 or more, such as 1 to 49 contiguous N-terminal amino acid residues. Reference to retaining binding to Ephrin B2 or B3 includes binding that is at least or at least about 5% of the level or degree of binding of the corresponding wild-type HeV-G, such as set forth in SEQ ID NO:23 or 24, 10% of the level or degree of binding of the corresponding wild-type HeV-G, such as set forth in SEQ ID NO:23 or 24, 15% of the level or degree of binding of the corresponding wild-type HeV-G, such as set forth in SEQ ID NO:23 or 24, 20% of the level or degree of binding of the corresponding wild-type HeV-G, such as set forth in SEQ ID NO:23 or 24, 25% of the level or degree of binding of the corresponding wild-type HeV-G, such as set forth in SEQ ID NO:23 or 24, 30% of the level or degree of binding of the corresponding wild-type HeV-G, such as set forth in SEQ ID NO:23 or 24, 35% of the level or degree of binding of the corresponding wild-type HeV-G, such as set forth in SEQ ID NO:23 or 24, 40% of the level or degree of binding of the corresponding wild-type HeV-G, such as set forth in SEQ ID NO:23 or 24, 45% of the level or degree of binding of the corresponding wild-type HeV-G, such as set forth in SEQ ID NO:23 or 24, 50% of the level or degree of binding of the corresponding wild-type HeV-G, such as set forth in SEQ ID NO:23 or 24, 55% of the level or degree of binding of the corresponding wild-type HeV-G, such as set forth in SEQ ID NO:23 or 24, 60% of the level or degree of binding of the corresponding wild-type HeV-G, such as set forth in SEQ ID NO:23 or 24, 65% of the level or degree of binding of the corresponding wild-type HeV-G, such as set forth in SEQ ID NO:23 or 24, 70% of the level or degree of binding of the corresponding wild-type HeV-G, such as set forth in SEQ ID NO:23 or 24, such as at least or at least about 75% of the level or degree of binding of the corresponding wild-type HeV-G, such as set forth in SEQ ID NO:23 or 24, such as at least or at least about 80% of the level or degree of binding of the corresponding wild-type NIV-G, such as set forth in SEQ ID NO:23 or 24, such as at least or at least about 85% of the level or degree of binding of the corresponding wild-type HeV-G, such as set forth in SEQ ID NO:23 or 24, such as at least or at least about 90% of the level or degree of binding of the corresponding wild-type HeV-G, such as set forth in SEQ ID NO:23 or 24, or such as at least or at least about 95% of the level or degree of binding of the corresponding wild-type HeV-G, such as set forth in SEQ ID NO:23 or 24.

In some embodiments, the G protein or the biologically thereof is a mutant G protein that exhibits reduced binding for the native binding partner of a wild-type G protein. In some embodiments, the mutant G protein or the biologically active portion thereof is a mutant of wild-type Niv-G and exhibits reduced binding to one or both of the native binding partners Ephrin B2 or Ephrin B3. In some embodiments, the mutant G-protein or the biologically active portion, such as a mutant NiV-G protein, exhibits reduced binding to the native binding partner. In some embodiments, the reduced binding to Ephrin B2 or Ephrin B3 is reduced by greater than at or about 5%, at or about 10%, at or about 15%, at or about 20%, at or about 25%, at or about 30%, at or about 40%, at or about 50%, at or about 60%, at or about 70%, at or about 80%, at or about 90%, or at or about 100%.

In some embodiments, the G protein contains one or more amino acid substitutions in a residue that is involved in the interaction with one or both of Ephrin B2 and Ephrin B3. In some embodiments, the amino acid substitutions correspond to mutations E501A, W504A, Q530A and E533A with reference to numbering set forth in SEQ ID NO:4.

In some embodiments, the G protein is a mutant G protein. In some embodiments, the G protein is a mutant G protein containing one or more amino acid substitutions selected from the group consisting of E501A, W504A, Q530A and E533A with reference to numbering set forth in SEQ ID NO:4. In some embodiments, the G protein is a mutant G protein that contains one or more amino acid substitutions elected from the group consisting of E501A, W504A, Q530A and E533A with reference to SEQ ID NO:4 and is a biologically active portion thereof containing an N-terminal truncation. In some embodiments, the mutant NiV-G protein or the biologically active portion thereof is truncated and lacks up to 5 contiguous amino acid residues at or near the N-terminus of the wild-type NiV-G protein (SEQ ID NO:4), 6 contiguous amino acid residues at or near the N-terminus of the wild-type NiV-G protein (SEQ ID NO:4), 7 contiguous amino acid residues at or near the N-terminus of the wild-type NiV-G protein (SEQ ID NO:4), 8 contiguous amino acid residues at or near the N-terminus of the wild-type NiV-G protein (SEQ ID NO:4), 9 contiguous amino acid residues at or near the N-terminus of the wild-type NiV-G protein (SEQ ID NO:4), up to 10 contiguous amino acid residues at or near the N-terminus of the wild-type NiV-G protein (SEQ ID NO:4), 11 contiguous amino acid residues at or near the N-terminus of the wild-type NiV-G protein (SEQ ID NO:4), 12 contiguous amino acid residues at or near the N-terminus of the wild-type NiV-G protein (SEQ ID NO:4), 13 contiguous amino acid residues at or near the N-terminus of the wild-type NiV-G protein (SEQ ID NO:4), 14 contiguous amino acid residues at or near the N-terminus of the wild-type NiV-G protein (SEQ ID NO:4), up to 15 contiguous amino acid residues at or near the N-terminus of the wild-type NiV-G protein (SEQ ID NO:4), 16 contiguous amino acid residues at or near the N-terminus of the wild-type NiV-G protein (SEQ ID NO:4), 17 contiguous amino acid residues at or near the N-terminus of the wild-type NiV-G protein (SEQ ID NO:4), 18 contiguous amino acid residues at or near the N-terminus of the wild-type NiV-G protein (SEQ ID NO:4), 19 contiguous amino acid residues at or near the N-terminus of the wild-type NiV-G protein (SEQ ID NO:4), up to 20 contiguous amino acid residues at or near the N-terminus of the wild-type NiV-G protein (SEQ ID NO:4), 21 contiguous amino acid residues at or near the N-terminus of the wild-type NiV-G protein (SEQ ID NO:4) 22 contiguous amino acid residues at or near the N-terminus of the wild-type NiV-G protein (SEQ ID NO:4), 23 contiguous amino acid residues at or near the N-terminus of the wild-type NiV-G protein (SEQ ID NO:4), 24 contiguous amino acid residues at or near the N-terminus of the wild-type NiV-G protein (SEQ ID NO:4), up to 25 contiguous amino acid residues at or near the N-terminus of the wild-type NiV-G protein (SEQ ID NO:4), 26 contiguous amino acid residues at or near the N-terminus of the wild-type NiV-G protein (SEQ ID NO:4), 27 contiguous amino acid residues at or near the N-terminus of the wild-type NiV-G protein (SEQ ID NO:4), 28 contiguous amino acid residues at or near the N-terminus of the wild-type NiV-G protein (SEQ ID NO:4), 29 contiguous amino acid residues at or near the N-terminus of the wild-type NiV-G protein (SEQ ID NO:4), up to 30 contiguous amino acid residues at or near the N-terminus of the wild-type NiV-G protein (SEQ ID NO: 4), up to 31 contiguous amino acid residues at or near the N-terminus of the wild-type NiV-G protein (SEQ ID NO:4), 32 contiguous amino acid residues at or near the N-terminus of the wild-type NiV-G protein (SEQ ID NO:4), 33 contiguous amino acid residues at or near the N-terminus of the wild-type NiV-G protein (SEQ ID NO:4) 34 contiguous amino acid residues at or near the N-terminus of the wild-type NiV-G protein (SEQ ID NO:4), 35 contiguous amino acid residues at or near the N-terminus of the wild-type NiV-G protein (SEQ ID NO:4) up to 36 contiguous amino acid residues at or near the N-terminus of the wild-type NiV-G protein (SEQ ID NO:4), up to 37 contiguous amino acid residues at or near the N-terminus of the wild-type NiV-G protein (SEQ ID NO:4), up to 38 contiguous amino acid residues at or near the N-terminus of the wild-type NiV-G protein (SEQ ID NO: 4), up to 39 contiguous amino acid residues at or near the N-terminus of the wild-type NiV-G protein (SEQ ID NO:4), or up to 40 contiguous amino acid residues at or near the N-terminus of the wild-type NiV-G protein (SEQ ID NO: 4).

In some embodiments, the mutant NiV-G protein has the amino acid sequence set forth in SEQ ID NO:17 or 18 or an amino acid sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:17 or 18. In particular embodiments, the G protein has the sequence of amino acids set forth in SEQ ID NO: 17 or 18. In some embodiments, the mutant NiV-G protein has the amino acid sequence set forth in SEQ ID NO:17 or an amino acid sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:17. In particular embodiments, the G protein has the sequence of amino acids set forth in SEQ ID NO 17. In some embodiments, the mutant NiV-G protein has the amino acid sequence set forth in SEQ ID NO:18 or an amino acid sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:18. In particular embodiments, the G protein has the sequence of amino acids set forth in SEQ ID NO 18.

In some embodiments, the G protein is a mutant G protein containing one or more amino acid substitutions selected from the group consisting of E501A, W504A, Q530A and E533A with reference to numbering set forth in SEQ ID NO:4. In some embodiments, the G protein is a mutant G protein that contains one or more amino acid substitutions elected from the group consisting of E501A, W504A, Q530A and E533A with reference to SEQ JD NO:4 and is a biologically active portion thereof containing an N-terminal truncation.

### 4. F Proteins

In some embodiments, the fusogen contains a protein with a hydrophobic fusion peptide domain. In some embodiments, the fusogen is or contains a F protein. In some embodiments, the fusogen contains a Henipavirus F protein molecule or biologically active portion thereof. In some embodiments, the Henipavirus F protein is a Hendra (Hev) virus F protein, a Nipah (NiV) virus F-protein, a Cedar (CedPV) virus F protein, a Mojiang virus F protein, a bat Paramyxovirus F protein, a Kumasi virus F protein, a Langya virus F protein, or a biologically active portion thereof.

Table 25 provides non-limiting examples of F proteins. In some embodiments, the N-terminal hydrophobic fusion peptide domain of the F protein molecule or biologically active portion thereof is exposed on the outside of lipid bilayer.

F proteins of henipaviruses are encoded as F₀ precursors containing a signal peptide (e.g. corresponding to amino acid residues 1-26 of SEQ ID NO:28). Following cleavage of the signal peptide, the mature F₀ (e.g. SEQ ID NO:29) is transported to the cell surface, then endocytosed and cleaved by cathepsin L into the mature fusogenic subunits F1 and F2. In some embodiments, the signal peptide comprises the amino acid sequence set forth in SEQ ID NO: 38. In some embodiments, the F₀ comprises the amino acid sequence of SEQ ID NO:41. In some embodiments, the F1 subunit comprises the sequence amino acid sequence set forth in SEQ ID NO:46. In some embodiments, the F2 subunit comprises the sequence amino acid sequence set forth in SEQ ID NO:39. The F1 and F2 subunits are associated by a disulfide bond and recycled back to the cell surface. The F1 subunit contains the fusion peptide domain located at the N terminus of the F1 subunit, where it is able to insert into a cell membrane to drive fusion. In some aspects, fusion is blocked by association of the F protein with G protein, until the G protein engages with a target molecule resulting in its disassociation from F and exposure of the fusion peptide to mediate membrane fusion.

Among different henipavirus species, the sequence and activity of the F protein is highly conserved. For examples, the F protein of NiV and HeV viruses share 89% amino acid sequence identity. Further, in some cases, the henipavirus F proteins exhibit compatibility with G proteins from other species to trigger fusion (Brandel-Tretheway et al. Journal of Virology. 2019. 93(13):e00577-19). In some aspects or the provided re-targeted viral vectors, the F protein is heterologous to the G protein, i.e. the F and G protein or biologically active portions are from different henipavirus species. For example, the F protein is from Hendra virus and the G protein is from Nipah virus. In other aspects, the F protein can be a chimeric F protein containing regions of F proteins from different species of Henipavirus. In some embodiments, switching a region of amino acid residues of the F protein from one species of Henipavirus to another can result in fusion to the G protein of the species comprising the amino acid insertion. (Brandel-Tretheway et al. Journal of Virology. 2019. 93(13):e00577-19). In some cases, the chimeric F protein contains an extracellular domain from one henipavirus species and a transmembrane and/or cytoplasmic domain from a different henipavirus species. For example, the F protein contains an extracellular domain of Hendra virus and a transmembrane/cytoplasmic domain of Nipah virus. F protein sequences disclosed herein are predominantly disclosed as expressed sequences including an N-terminal signal sequence. As such N-terminal signal sequences are commonly cleaved co- or post-translationally, the mature protein sequences for all F protein sequences disclosed herein are also contemplated as lacking the N-terminal signal sequence.

TABLE 25
F proteins

| | | | SEQ ID |
|---|---|---|---|
| | | | (without |
| Full Gene | | | signal |
| Name | Sequence | SEQ ID | sequence) |
| Hendra | MATQEVRLKCLLCGIIVLVLSLEGLGILHYEKLSKIG | 28 | 29 |
| virus F | LVKGITRKYKIKSNPLTKDIVIKMIPNVSNVSKCTGT | | |
| Protein | VMENYKSRLTGILSPIKGAIELYNNNTHDLVGDVKL | | |
| | AGVVMAGIAIGIATAAQITAGVALYEAMKNADNIN | | |
| | KLKSSIESTNEAVVKLQETAEKTVYVLTALQDYINT | | |
| | NLVPTIDQISCKQTELALDLALSKYLSDLLFVFGPNL | | |
| | QDPVSNSMTIQAISQAFGGNYETLLRTLGYATEDFD | | |
| | DLLESDSIAGQIVYVDLSSYYIIVRVYFPILTEIQQAY | | |
| | VQELLPVSFNNDNSEWISIVPNFVLIRNTLISNIEVK | | |
| | YCLITKKSVICNQDYATPMTASVRECLTGSTDKCPR | | |
| | ELVVSSHVPRFALSGGVLFANCISVTCQCQTTGRAIS | | |
| | QSGEQTLLMIDNTTCTTVVLGNIIISLGKYLGSINYN | | |
| | SESIAVGPPVYTDKVDISSQISSMNQSLQQSKDYIKE | | |
| | AQKILDTVNPSLISMLSMIILYVLSIAALCIGLITFISF | | |
| | VIVEKKRGNYSRLDDRQVRPVSNGDLYYIGT | | |
| Nipah virus | MVVILDKRCYCNLLILILMISECSVGILHYEKLSKIG | 30 | 31 |
| F Protein | LVKGVTRKYKIKSNPLTKDIVIKMIPNVSNMSQCTG | | |
| | SVMENYKTRLNGILTPIKGALEIYKNNTHDLVGDVR | | |
| | LAGVIMAGVAIGIATAAQITAGVALYEAMKNADNIN | | |
| | KLKSSIESTNEAVVKLQETAEKTVYVLTALQDYINT | | |
| | NLVPTIDKISCKQTELSLDLALSKYLSDLLFVFGPNL | | |
| | QDPVSNSMTIQAISQAFGGNYETLLRTLGYATEDFD | | |
| | DLLESDSITGQIIYVDLSSYYIIVRVYFPILTEIQQAYI | | |
| | QELLPVSFNNDNSEWISIVPNFILVRNTLISNIEIGFCL | | |
| | ITKRSVICNQDYATPMTNNMRECLTGSTEKCPRELV | | |
| | VSSHVPRFALSNGVLFANCISVTCQCQTTGRAISQS | | |
| | GEQTLLMIDNTTCPTAVLGNVIISLGKYLGSVNYNS | | |
| | EGIAIGPPVFTDKVDISSQISSMNQSLQQSKDYIKEA | | |
| | QRLLDTVNPSLISMLSMIILYVLSIASLCIGLITFISFII | | |
| | VEKKRNTYSRLEDRRVRPTSSGDLYYIGT | | |
| Cedar Virus | MSNKRTTVLIIISYTLFYLNNAAIVGFDFDKLNKIGV | 32 | 33 |
| F Protein | VQGRVLNYKIKGDPMTKDLVLKFIPNIVNITECVRE | | |
| | PLSRYNETVRRLLLPIHNMLGLYLNNTNAKMTGLM | | |
| | IAGVIMGGIAIGIATAAQITAGFALYEAKKNTENIQK | | |
| | LTDSIMKTQDSIDKLTDSVGTSILILNKLQTYINNQL | | |
| | VPNLELLSCRQNKIEFDLMLTKYLVDLMTVIGPNIN | | |
| | NPVNKDMTIQSLSLLFDGNYDIMMSELGYTPQDFL | | |
| | DLIESKSITGQIIYVDMENLYVVIRTYLPTLIEVPDAQ | | |
| | IYEFNKITMSSNGGEYLSTIPNFILIRGNYMSNIDVAT | | |
| | CYMTKASVICNQDYSLPMSQNLRSCYQGETEYCPV | | |
| | EAVIASHSPRFALTNGVIFANCINTICRCQDNGKTITQ | | |
| | NINQFVSMIDNSTCNDVMVDKFTIKVGKYMGRKDI | | |
| | NNINIQIGPQIIIDKVDLSNEINKMNQSLKDSIFYLRE | | |
| | AKRILDSVNISLISPSVQLFLIIISVLSFIILLIIIVYLYC | | |
| | KSKHSYKYNKFIDDPDYYNDYKRERINGKASKSNN | | |
| | IYYVGD | | |
| Mojiang | MALNKNMFSSLFLGYLLVYATTVQSSIHYDSLSKV | 34 | 35 |
| virus, | GVIKGLTYNYKIKGSPSTKLMVVKLIPNIDSVKNCT | | |
| Tongguan 1 | QKQYDEYKNLVRKALEPVKMAIDTMLNNVKSGNN | | |
| F Protcin | KYRFAGAIMAGVALGVATAATVTAGIALHRSNENA | | |
| | QAIANMKSAIQNTNEAVKQLQLANKQTLAVIDTIRG | | |
| | EINNNIIPVINQLSCDTIGLSVGIRLTQYYSEIITAFGP | | |
| | ALQNPVNTRITIQAISSVFNGNFDELLKIMGYTSGD | | |
| | LYEILHSELIRGNIIDVDVDAGYIALEIEFPNLTLVPN | | |
| | AVVQELMPISYNIDGDEWVTLVPRFVLTRTTLLSNI | | |
| | DTSRCTITDSSVICDNDYALPMSHELIGCLQGDTSK | | |
| | CAREKVVSSYVPKFALSDGLVYANCLNTICRCMDT | | |
| | DTPISQSLGATVSLLDNKRCSVYQVGDVLISVGSYL | | |
| | GDGEYNADNVELGPPIVIDKIDIGNQLAGINQTLQE | | |
| | AEDYIEKSEEFLKGVNPSIITLGSMVVLYIFMILIAIV | | |
| | SVIALVLSIKLTVKGNVVRQQFTYTQHVPSMENINY | | |
| | VSH | | |
| Bat | MKKKTDNPTISKRGHNHSRGIKSRALLRETDNYSN | 36 | 37 |
| Paramyxovirus | GLIVENLVRNCHHPSKNNLNYTKTQKRDSTIPYRVE | | |
| Eid_hel/GH- | ERKGHYPKIKHLIDKSYKHIKRGKRRNGHNGNIITII | | |
| M74a/GHA/ | LLLILILKTQMSEGAIHYETLSKIGLIKGITREYKVK | | |
| 2009 F | GTPSSKDIVIKLIPNVTGLNKCTNISMENYKEQLDKI | | |
| protein | LIPINNIIELYANSTKSAPGNARFAGVIIAGVALGVAA | | |
| | AAQITAGIALHEARQNAERINLLKDSISATNNAVAEL | | |
| | QEATGGIVNVITGMQDYINTNLVPQIDKLQCSQIKT | | |
| | ALDISLSQYYSEILTVFGPNLQNPVTTSMSIQAISQSF | | |
| | GGNIDLLLNLLGYTANDLLDLLESKSITGQITYINLE | | |
| | HYFMVIRVYYPIMTTISNAYVQELIKISFNVDGSEW | | |
| | VSLVPSYILIRNSYLSNIDISECLITKNSVICRHDFAM | | |
| | PMSYTLKECLTGDTEKCPREAVVTSYVPRFAISGGV | | |
| | IYANCLSTTCQCYQTGKVIAQDGSQTLMMIDNQTC | | |
| | SIVRIEEILISTGKYLGSQEYNTMHVSVGNPVFTDKL | | |
| | DITSQISNINQSIEQSKFYLDKSKAILDKINLNLIGSV | | |
| | PISILFIIAILSLILSIITFVIVMIIVRRYNKYTPLINSDPS | | |
| | SRRSTIQDVYIIPNPGEHSIRSAARSIDRDRD | | |

In some embodiments, the F protein is encoded by a nucleotide sequence that encodes the sequence set forth by any one of SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, or SEQ ID NO:37, or is a functionally active variant or a biologically active portion thereof that has a sequence that is at least at or about 80%, at least at or about 85%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identical to any one of SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, or SEQ ID NO:37. In some embodiments, the F protein is encoded by a nucleotide sequence that encodes the sequence set forth by any one of SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, or SEQ ID NO:37.

In particular embodiments, the F protein or the functionally active variant or biologically active portion thereof retains fusogenic activity in conjunction with a Henipavirus G protein, such as a G protein set forth above (e.g. NiV-G or HeV-G). Fusogenic activity includes the activity of the F protein in conjunction with a G protein to promote or facilitate fusion of two membrane lumens, such as the lumen of the targeted viral vector having embedded in its lipid bilayer a henipavirus F and G protein, and a cytoplasm of a target cell, e.g. a cell that contains a surface receptor or molecule that is recognized or bound by the targeted envelope protein. In some embodiments, the F protein and G protein are from the same Henipavirus species (e.g. NiV-G and NiV-F). In some embodiments, the F protein and G protein are from different Henipavirus species (e.g. NiV-G and HeV-F). In particular embodiments, the F protein of the functionally active variant or biologically active portion retains the cleavage site cleaved by cathepsin L (e.g. corresponding to the cleavage site between amino acids 109-110 of SEQ ID NO:30).

In particular embodiments, the F protein has the sequence of amino acids set forth in SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, or SEQ ID NO:37, or is a functionally active variant thereof or a biologically active portion thereof that retains fusogenic activity. In some embodiments, the functionally active variant comprises an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, or SEQ ID NO:37, and retains fusogenic activity in conjunction with a Henipavirus G protein (e.g., NiV-G or HeV-G). In some embodiments, the biologically active portion has an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, or SEQ ID NO:37.

Reference to retaining fusogenic activity includes activity (in conjunction with a Henipavirus G protein) that between at or about 10% and at or about 150% or more of the level or degree of binding of the corresponding wild-type F protein, such as set forth in SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, or SEQ ID NO:37, such as at least or at least about 10% of the level or degree of fusogenic activity of the corresponding wild-type F protein, such as at least or at least about 15% of the level or degree of fusogenic activity of the corresponding wild-type F protein, such as at least or at least about 20% of the level or degree of fusogenic activity of the corresponding wild-type F protein, such as at least or at least about 25% of the level or degree of fusogenic activity of the corresponding wild-type F protein, such as at least or at least about 30% of the level or degree of fusogenic activity of the corresponding wild-type F protein, such as at least or at least about 35% of the level or degree of fusogenic activity of the corresponding wild-type F protein, such as at least or at least about 40% of the level or degree of fusogenic activity of the corresponding wild-type F protein, such as at least or at least about 45% of the level or degree of fusogenic activity of the corresponding wild-type F protein, such as at least or at least about 50% of the level or degree of fusogenic activity of the corresponding wild-type F protein, such as at least or at least about 55% of the level or degree of fusogenic activity of the corresponding wild-type f protein, such as at least or at least about 60% of the level or degree of fusogenic activity of the corresponding wild-type F protein, such as at least or at least about 65% of the level or degree of fusogenic activity of the corresponding wild-type F protein, such as at least or at least about 70% of the level or degree of fusogenic activity of the corresponding wild-type F protein, such as at least or at least about 75% of the level or degree of fusogenic activity of the corresponding wild-type F protein, such as at least or at least about 80% of the level or degree of fusogenic activity of the corresponding wild-type F protein, such as at least or at least about 85% of the level or degree of fusogenic activity of the corresponding wild-type F protein, such as at least or at least about 90% of the level or degree of fusogenic activity of the corresponding wild-type F protein, such as at least or at least about 95% of the level or degree of fusogenic activity of the corresponding wild-type F protein, such as at least or at least about 100% of the level or degree of fusogenic activity of the corresponding wild-type F protein, or such as at least or at least about 120% of the level or degree of fusogenic activity of the corresponding wild-type F protein.

In some embodiments, the F protein is a mutant F protein that is a functionally active fragment or a biologically active portion containing one or more amino acid mutations, such as one or more amino acid insertions, deletions, substitutions or truncations. In some embodiments, the mutations described herein relate to amino acid insertions, deletions, substitutions or truncations of amino acids compared to a reference F protein sequence. In some embodiments, the reference F protein sequence is the wild-type sequence of an F protein or a biologically active portion thereof. In some embodiments, the mutant F protein or the biologically active portion thereof is a mutant of a wild-type Hendra (Hey) virus F protein, a Nipah (NiV) virus F-protein, a Cedar (CedPV) virus F protein, a Mojiang virus F protein, a bat Paramyxovirus F protein, a Kumasi virus F protein, or a Langya virus F protein. In some embodiments, the wild-type F protein is encoded by a sequence of nucleotides that encodes any one of SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, or SEQ ID NO:37.

In some embodiments, the mutant F protein is a biologically active portion of a wild-type F protein that is an N-terminally and/or C-terminally truncated fragment. In some embodiments, the mutant F protein or the biologically active portion of a wild-type F protein thereof comprises one or more amino acid substitutions. In some embodiments, the mutations described herein can improve transduction efficiency. In some embodiments, the mutations described herein can increase fusogenic capacity. Exemplary mutations include any as described, see e.g. Khetawat and Broder 2010 Virology Journal 7:312; Witting et al. 2013 Gene Therapy 20:997-1005; published international; patent application No. WO/2013/148327.

In some embodiments, the mutant F protein is a biologically active portion that is truncated and lacks up to 20 contiguous amino acid residues at or near the C-terminus of the wild-type F protein, such as a wild-type F protein encoded by a sequence of nucleotides encoding the F protein set forth in any one of SEQ ID NOS: 28-37. In some embodiments, the mutant F protein is truncated and lacks up to 20 contiguous amino acids, such as up to 19, 18, 17, 16, 15, 14, 13, 12, 11, 10, 9, 8, 7, 6, 5, 4, 3, 2, or 1 contiguous amino acids at the C-terminus of the wild-type F protein. In some embodiments, the mutant F protein comprises the sequence set forth in SEQ ID NO:15. In some embodiments, the mutant F protein comprises the sequence set forth in SEQ ID NO:20. In some embodiments, the mutant F protein is truncated and lacks up to 19 contiguous amino acids, such as up to 18, 17, 16, 15, 14, 13, 12, 11, 10, 9, 8, 7, 6, 5, 4, 3, 2, or 1 contiguous amino acids at the C-terminus of the wild-type F protein.

In some embodiments, the F protein or the functionally active variant or biologically active portion thereof comprises an F1 subunit or a fusogenic portion thereof. In some embodiments, the F1 subunit is a proteolytically cleaved portion of the F₀ precursor. In some embodiments, the F₀ precursor is inactive. In some embodiments, the cleavage of the F₀ precursor forms a disulfide-linked F1+F2 heterodimer. In some embodiments, the cleavage exposes the fusion peptide and produces a mature F protein. In some embodiments, the cleavage occurs at or around a single basic residue. In some embodiments, the cleavage occurs at Arginine 109 of NiV-F protein. In some embodiments, cleavage occurs at Lysine 109 of the Hendra virus F protein.

In some embodiments, the F protein is a wild-type Nipah virus F (NiV-F) protein or is a functionally active variant or biologically active portion thereof. In some embodiments, the F₀ precursor is encoded by a sequence of nucleotides encoding the sequence set forth in SEQ ID NO:20. The encoding nucleic acid can encode a signal peptide sequence that has the sequence MVVILDKRCY CNLLILILMI SECSVG (SEQ ID NO:38). In some examples, the F protein is cleaved into an F1 subunit comprising the sequence set forth in SEQ ID NO:46 and an F2 subunit comprising the sequence set forth in SEQ ID NO:39.

In some embodiments, the F protein is a NiV-F protein that is encoded by a sequence of nucleotides encoding the sequence set forth in SEQ ID NO:30, or is a functionally active variant or biologically active portion thereof that has an amino acid sequence having at least at or about 80%, at least at or about 81%, at least at or about 82%, at least at or about 83%, at least at or about 84%, at least at or about 85%, at or about 86%, at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:30. In some embodiments, the F protein is a NiV-F protein that is encoded by a sequence of nucleotides encoding the sequence set forth in SEQ ID NO:30. In some embodiments, the NiV-F-protein has the sequence of set forth in 30, or is a functionally active variant or a biologically active portion thereof that has an amino acid sequence having at least at or about 80%, at least at or about 81%, at least at or about 82%, at least at or about 83%, at least at or about 84%, at least at or about 85%, at or about 86%, at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to 30. In some embodiments, the NiV-F-protein has the sequence of set forth in 30. In In particular embodiments, the F protein or the functionally active variant or biologically active portion thereof retains the cleavage site cleaved by cathepsin L.

In some embodiments, the F protein or the functionally active variant or the biologically active portion thereof includes an F1 subunit that has the sequence set forth in SEQ ID NO:46, or an amino acid sequence having, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:46.

In some embodiments, the F protein or the functionally active variant or biologically active portion thereof includes an F2 subunit that has the sequence set forth in SEQ ID NO:39, or an amino acid sequence having, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:39.

In some embodiments, the F protein or the functionally active variant or the biologically active portion thereof includes an F1 subunit that has the sequence set forth in SEQ ID NO:46, or an amino acid sequence having, at least at or about 80%, at least at or about 81%, at least at or about 82%, at least at or about 83%, at least at or about 84%, at least at or about 85%, at or about 86%, at least at or about 87%, at least at or about 88%, or at least at or about 89% at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:46.

In some embodiments, the F protein or the functionally active variant or biologically active portion thereof includes an F2 subunit that has the sequence set forth in SEQ ID NO:39, or an amino acid sequence having, at least at or about 80%, at least at or about 81%, at least at or about 82%, at least at or about 83%, at least at or about 84%, at least at or about 85%, at or about 86%, at least at or about 87%, at least at or about 88%, or at least at or about 89% at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:39.

In some embodiments, the F protein is a mutant NiV-F protein that is a biologically active portion thereof that is truncated and lacks up to 20 contiguous amino acid residues at or near the C-terminus of the wild-type NiV-F protein (e.g. set forth SEQ ID NO:40). In some embodiments, the mutant NiV-F protein comprises an amino acid sequence set forth in SEQ ID NO:20. In some embodiments, the mutant NiV-F protein has a sequence that has at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:20. In some embodiments, the mutant F protein contains an F1 protein that has the sequence set forth in SEQ ID NO:46. In some embodiments, the mutant F protein has a sequence that has at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:46.

In some embodiments, the F protein is a mutant NiV-F protein that is a biologically active portion thereof that comprises a 20 amino acid truncation at or near the C-terminus of the wild-type NiV-F protein (SEQ ID NO:40); and a point mutation on an N-linked glycosylation site. In some embodiments, the mutant NiV-F protein comprises an amino acid sequence set forth in SEQ ID NO:15. In some embodiments, the mutant NiV-F protein has a sequence that has at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:15.

In some embodiments, the F protein is a mutant NiV-F protein that is a biologically active portion thereof that comprises a 25 amino acid truncation at or near the C-terminus of the wild-type NiV-F protein (SEQ ID NO:40). In some embodiments, the F protein is a mutant NiV-F protein that is a biologically active portion thereof that comprises a 22 amino acid truncation at or near the C-terminus of the wild-type NiV-F protein (SEQ ID NO:40). In some embodiments, the NiV-F protein is encoded by a nucleotide sequence that encodes the sequence set forth in SEQ ID NO:20. In some embodiments, the NiV-F proteins is encoded by a nucleotide sequence that encodes sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:20.

In some embodiments, the F protein is a mutant NiV-F protein that is a biologically active portion thereof that comprises a 22 amino acid truncation at or near the C-terminus of the wild-type NiV-F protein (SEQ ID NO:40). In some embodiments, the NiV-F protein comprises the amino acid sequence set forth in SEQ ID NO:21, or an amino acid sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:21. In some embodiments, the NiV-F protein is encoded by a nucleotide sequence that encodes the sequence set forth in SEQ ID NO:21. In some embodiments, the NiV-F proteins is encoded by a nucleotide sequence that encodes sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:21.

In some embodiments where a VLP includes a fusogen that contains a G or H protein and an F protein (e.g., a G or H protein and F protein from a Paramyxovirus (e.g., Morbillivirus or Henipavirus) G or H protein or a biologically active portion thereof), the VLP also comprises a gag protein (e.g. an MLV-gag protein). In some embodiments, the MLV-gag protein is part of a fusion protein, where the the fusion protein is a cleavable fusion protein between (i) a viral structural protein (e.g. GAG (e.g. MLV-gag or HIV-gag)) and (ii) a nuclease protein (e.g. Cas protein (e.g. any of the Cas protein described herein). In some embodiments where the VLP includes fusogen that contains a G or H protein and an F protein and a gag protein (e.g. an MLV-gag protein), production of such a VLP includes varying the ratio of polynucleotide encoding the G or H protein and an F protein and the polynucleotide encoding the gag protein. In some embodiments, the ratio of polynucleotide encoding the G or H protein and an F protein to the polynucleotide encoding the gag protein includes a 1:1, 1:2, 1:3, 1:4, or 1:5 ratio.

### C. Targeting Agent

In some embodiments, the viral vector includes a targeting agent that binds to a target molecule. In some embodiments, the target molecule is expressed on a target cell. In some embodiments, the targeting agent targets the viral vector to the target cell. In some embodiments, the viral vector preferentially targets a target cell compared to a non-target cell. In some embodiments, the viral vector preferentially fuses with a target cell compared to a non-target cell.

In some embodiments, the fusogen is fused to the targeting agent of the viral vector. In some aspects, the fusogen is retargeted by the targeting agent to display altered tropism.

In some embodiment, the targeting agent is linked to an exposed domain of the fusogen. In some embodiments, the exposed domain is the N-terminus. In some embodiments, the exposed domain is the C-terminus. In some embodiments, the targeting agent is fused to the fusogen directly. In some embodiments, the targeting agent is fused to the fusogen via a linker. In some embodiments, the linker is a peptide linker. In some embodiments, the targeting agent and fusogen are fused together, directly or indirectly, such as via the linker, in a fusion protein.

In some embodiments, the targeting agent is fused to a Paramyxovirus attachment protein comprised by the fusogen. In some embodiments, the targeting agent is fused to the C-terminus of the Paramyxovirus attachment protein. In some embodiments, the targeting agent is fused to the Paramyxovirus attachment protein directly. In some embodiments, the targeting agent is fused to the Paramyxovirus attachment protein via a linker. In some embodiments, the linker is a peptide linker. In some embodiments, the targeting agent and Paramyxovirus attachment protein are fused together, directly or indirectly, such as via the linker, in a fusion protein.

In some embodiments, the Paramyxovirus attachment protein is a Paramyxovirus G or H protein. In some embodiments, the Paramyxovirus attachment protein is a Paramyxovirus G protein. In some embodiments, the Paramyxovirus attachment protein is a Paramyxovirus H protein. In some embodiments, the Paramyxovirus attachment protein is any described in Section IT-B-2-b. In some embodiments, the Paramyxovirus attachment protein is a mutated variant having reduced binding to its native receptor.

In other embodiments, the targeting agent is fused to a separate transmembrane domain incorporated into the lipid bilayer, not the fusogen. The transmembrane domain may be any naturally-occurring or non-naturally occurring transmembrane domain. The transmembrane domain may be a transmembrane domain of a receptor, a transmembrane protein, preferably a viral transmembrane protein, a fragment of a transmembrane protein, a transmembrane peptide or a variant thereof, such as a genetically modified transmembrane domain of a receptor, a genetically modified transmembrane protein, a genetically modified fragment of a transmembrane protein, or a genetically modified transmembrane peptide. Examples of transmembrane domains are the transmembrane domain (TMD) of the platelet-derived growth factor receptor (PDGFR), the transmembrane domain of CD34, or the VSVG glycoprotein transmembrane domain.

In some embodiments, the C-terminus of the transmembrane domain is fused, directly or indirectly (for example via a linker, such as a peptide linker), to the N-terminus of the targeting agent.

In particular embodiments, the linker is a flexible peptide linker. In some such embodiments, the linker is 1-20 amino acids, such as 1-20 amino acids predominantly composed of glycine. In some embodiments, the linker is 1-20 amino acids, such as 1-20 amino acids predominantly composed of glycine and serine. In some embodiments, the linker is a flexible peptide linker containing amino acids Glycine and Serine, referred to as GS-linkers. In some embodiments, the peptide linker includes the sequences GS, GGS, GGGGS (SEQ ID NO:263), GGGGGS (SEQ ID NO:264), or combinations thereof. In some embodiments, the polypeptide linker has the sequence (GGS)n (SEQ IDNO:268), wherein n is 1 to 10. In some embodiments, the polypeptide linker has the sequence (GGGGS)n, (SEQ ID NO:265) wherein n is 1 to 10. In some embodiments, the polypeptide linker has the sequence (GGGGGS)n (SEQ ID NO:266), wherein n is 1 to 6. In some embodiments, the peptide linker is (GmS)n (SEQ ID NO:267), wherein each of m and n is an integer between 1 to 4, inclusive.

As used herein, a “target cell” refers to a cell of a type that is specifically targeted by the viral vector. In some embodiments, the target molecule is expressed on the target cell. In some embodiments, the target cell includes of a B cell, a T cell, a natural killer cell, an islet cell, a glial progenitor cell, a cardiac cell, a blood cell, a hepatocyte, a CD34+ progenitor cell, a stem cell, or an induced pluripotent stem cell.

As used herein, a “non-target cell” refers to a cell of a type to which targeting of the viral vector is not desired. In some embodiments, the target molecule is expressed at higher levels on target cells than on non-target cells. In some embodiments, the target molecule is not expressed on the non-target cell.

In some embodiments, the target cell is a thymocyte. In some embodiments, the target cell is a natural killer (NK) cell. In some embodiments, the thymocyte expresses CD4 or CD8. In some embodiments, the thymocyte does not express CD4 or CD8. In some embodiments, the natural killer (NK) cell is a cell that expresses CD56.

In some embodiments, one or more fusogens comprise at least one fusogen that has a tropism for B cells. In some embodiments, one or more fusogens comprise at least one fusogen that has a tropism for T cells. In some embodiments, one or more fusogens comprise at least one fusogen that has a tropism for islet cells. In some embodiments, one or more fusogens comprise at least one fusogen that has a tropism for cardiac cells. In some embodiments, one or more fusogens comprise at least one fusogen that has an endogenous tropism. In some embodiments, one or more fusogens comprise at least one fusogen that has engineered tropism.

In some embodiments, one or more fusogens comprise one or more chimeric proteins. In some embodiments, one or more chimeric proteins comprise at least one chimeric protein that comprises a paramyxovirus envelope protein or biologically active portion thereof. In some embodiments, one or more chimeric proteins comprise at least one chimeric protein that comprises an scFV. In some embodiments, one or more chimeric proteins comprise at least one chimeric protein that comprises (i) a paramyxovirus envelope protein or biologically active portion thereof and (ii) an scFV. In some embodiments, an scFV targets an antigen present on the surface of a B cell, a T cell, a natural killer cell, an islet cell, a glial progenitor cell, a cardiac cell, a blood cell, a hepatocyte, a stem cell, or an induced pluripotent stem cell.

In some embodiments, the target cell is a T cell. In some embodiments, the T cell is a CD3+ T cell, a CD4+ T cell, a CDS+ T cell, a naive T cell, a regulatory T (Treg) cell, a non-regulatory T cell, a Th1 cell, a Th2 cell, a Th9 cell, a Th17 cell, a T-follicular helper (Tfh) cell, a cytotoxic T lymphocyte (CTL), an effector T (Teff) cell, a central memory T cell, an effector memory T cell, an effector memory T cell expressing CD45RA (TEMRA cell), a tissue-resident memory (Trm) cell, a virtual memory T cell, an innate memory T cell, a memory stem cell (Tse), or a γδ T cell. In some embodiments, the T cell is a cytotoxic T cell, a helper T cell, a memory T cell, a regulatory T cell, or a tumor infiltrating lymphocyte. In some embodiments, the T cell is a human T cell. In some embodiments, the targeting agent targets CD8, optionally, the targeting agent is an scFv that targets CD8. In some embodiments, the targeting agent targets CD4, optionally, the targeting agent is an scFv that targets CD4.

In some embodiments, an scFV targets an antigen present on the surface of a B cell. In some embodiments, an scFV targets an antigen present on the surface of a T cell. In some embodiments, an scFV targets CD8. In some embodiments, an scFV targets CD4. Sequences of CD4-targeting scFVs, CDR domains, V_{H}, and V_{L} regions are provided in Tables 25-28. Sequences of CD8-targeting scFVs, CDR domains, V_{H}, and V_{L} regions are provided in Tables 27-32.

In some embodiments, an scFV targets an antigen present on the surface of an islet cell. In some embodiments, an islet cell is an alpha cell, a beta cell, or a delta cell. In some embodiments, an islet cell is a beta cell.

TABLE 25
HCDR in Kabat Numbering Scheme

| CD4 | H-CDR1 | | H-CDR2 | | H-CDR3 | |
|---|---|---|---|---|---|---|
| Binder | Sequence | SEQ ID NO: | Sequence | SEQ ID NO: | Sequence | SEQ ID NO: |
| 1 | SYWIE | 553 | EILPGSGSTSYNEKFKG | 557 | RGYGYDEGFDY | 561 |
| 2 | DYVIS | 554 | EIYPGSGSSYYNEKFKG | 558 | PGDLGFAY | 562 |
| 3 | THWMH | 555 | MINPSDGVTYYAQTFQG | 559 | EYYGEGFDY | 563 |
| 4 | GYWMY | 556 | AISPGGGSTYYPDSVKG | 560 | SLTATHTYEYDY | 564 |

TABLE 26
LCDR in Kabat Numbering Scheme

| CD4 | L-CDR1 | | L-CDR2 | | L-CDR3 | |
|---|---|---|---|---|---|---|
| Binder | Sequence | SEQ ID NO: | Sequence | SEQ ID NO: | Sequence | SEQ ID NO: |
| 1 | ASQDINSYLS | 565 | RANRLVD | 568 | LQYDEFPPT | 571 |
| 2 | ASQSVDYDGDSYMN | 566 | AASNLES | 569 | QQSNKDPFT | 572 |
| 3 | RASQGISNYLA | 567 | SASNLQS | 570 | QQSYSTPLT | 573 |

TABLE 27
VH Sequences

| CD4 | | |
|---|---|---|
| Binder | VH Sequence | SEQ ID NO: |
| 1 | QVQLQQSGAELMKPGASVKISCKATGYTFS | 574 |
| | SYWIEWVKQRPGHGLEWIGEILPGSGSTSY | |
| | NEKFKGKATFTADTSSNTAYMQLSSLTSED | |
| | SAVYYCARRGYGYDEGFDYWGQGTTLTVSS | |
| 2 | QVQLQQSGPELVKPGASVKMSCKASGYTFT | 575 |
| | DYVISWVRQAPGQGLEWIGEIYPGSGSSYY | |
| | NEKFKGRATLTADKSSNTAYMQLSSLRSED | |
| | SAVYFCARPGDLGFAYWGQGTLVTVSS | |
| 3 | QVQLVQSGAEVKKPGASVKVSCKASGYSLI | 576 |
| | THWMHWVRQAPGQGLEWMGMINPSDGVTYY | |
| | AQTFQGRVTMTRDTSTSTVYMELSSLRSED | |
| | TAVYYCAREYYGEGFDYWGQGTLVTVSS | |
| 4 | EVQLVESGGGLVQSGGSLRLSCAASGFTFS | 577 |
| | GYWMYWVRQAPGKGLEWVSAISPGGGSTYY | |
| | PDSVKGRFTISRDNAKNTLYLQMNSLEPED | |
| | TALYYCASSLTATHTYEYDYWGQGTQVTVS | |
| | S | |

TABLE 28
VL Sequences

| CD4 | | |
|---|---|---|
| Binder | VL Sequence | SEQ ID NO: |
| 1 | DIKMTQSPSSMYASLGERVTITCKASQDIN | 578 |
| | SYLSWFQQKPGKSPKTLIYRANRLVDGVPS | |
| | RFSGSGSGQDYSLTISSLEYEDMGIYYCLQ | |
| | YDEFPPTFGAGTKLELKR | |
| 2 | DIVLTQSPSSLAVSLGQRATISCKASQSVD | 579 |
| | YDGDSYMNWYQQKPGQPPKLLIYAASNLES | |
| | GIPARFSGSGSGTDFTLTIHPVEEEDAATY | |
| | YCQQSNKDPFTFGGGTKLELKR | |
| 3 | DIQMTQSPSSLSASVGDRVTITCRASQGIS | 580 |
| | NYLAWYQQKPGKAPKLLIYSASNLQSGVPS | |
| | RFSGSGSGTDFTLTISSLQPEDFATYYCQQ | |
| | SYSTPLTFGGGTKVEIKR* | |

TABLE 29
HCDRS in Kabat Numbering Scheme

| CD8 | H-CDR1 | | H-CDR2 | | H-CDR3 | |
|---|---|---|---|---|---|---|
| Binder | Sequence | SEQ ID NO: | Sequence | SEQ ID NO: | Sequence | SEQ ID NO: |
| 1 | SYAIS | 581 | IIDPSDGNTNYAQNFQG | 585 | ERAAAGYYYYMDV | 589 |
| 2 | DYYIQ | 582 | WINPNSGGTSYAQKFQG | 586 | EGDYYYGMDA | 590 |
| 3 | SYYMH | 583 | GFDPEDGETIYAQKFQG | 587 | DQGWGMDV | 591 |
| 4 | NHYMH | 584 | WMNPNSGNTGYAQKFQG | 588 | SESGSDLDY | 592 |

TABLE 30
LCDRS in Kabat Numbering Scheme

| CD8 | L-CDR1 | | L-CDR2 | | L-CDR3 | |
|---|---|---|---|---|---|---|
| Binder | Sequence | SEQ ID NO: | Sequence | SEQ ID NO: | Sequence | SEQ ID NO: |
| 1 | RASQSISSYLN | 593 | AASSLQS | 597 | QQSYSTPLT | 601 |
| 2 | RSSQSLLHSNGYNYLD | 594 | LGSNRAS | 598 | MQGLQTPHT | 602 |
| 3 | RASQSISSYLN | 595 | AASSLQS | 599 | QQTYSTPYT | 603 |
| 4 | RASQTIGNYVN | 596 | GASNLHT | 600 | QQTYSAPLT | 604 |

TABLE 31
VH Sequences

| CD8 | | SEQ |
|---|---|---|
| Binder | VH Sequence | ID NO: |
| 1 | QVQLVQSGAEVKKPGASVKVSCKASGGTFSSYAI | 605 |
| | SWVRQAPGQGLEWMGIIDPSDGNTNYAQNFQGRV | |
| | TMTRDTSTSTVYMELSSLRSEDTAVYYCAKERAA | |
| | AGYYYYMDVWGQGTTVTVSS | |
| 2 | QVQLVQSGAEVKKPGASVKVSCKASGYTFTDYYI | 606 |
| | QWVRQAPGQGLEWMGWINPNSGGTSYAQKFQGRV | |
| | TMTRDTSTSTVYMELSSLRSEDTAVYYCAKEGDY | |
| | YYGMDAWGQGTMVTVSS | |
| 3 | QVQLVQSGAEVKKPGASVKVSCKASGYTFTSYYM | 607 |
| | HWVRQAPGQGLEWMGGFDPEDGETIYAQKFQGRV | |
| | TMTRDTSTSTVYMELSSLRSEDTAVYYCARDQGW | |
| | GMDVWGQGTTVTVSS | |
| 4 | QVQLVQSGAEVKKPGASVKVSCKASGYTFTNHYM | 608 |
| | HWVRQAPGQGLEWMGWMNPNSGNTGYAQKFQGRV | |
| | TMTRDTSTSTVYMELSSLRSEDTAVYYCASSESG | |
| | SDLDYWGQGTLVTVSS | |

TABLE 32
VL Sequences

| CD8 | | SEQ |
|---|---|---|
| Binder | VH Sequence | ID NO: |
| 1 | DIQMTQSPSSLSASVGDRVTITCRASQSISS | 609 |
| | YLNWYQQKPGKAPKLLIYAASSLQSGVPSRF | |
| | SGSGSGTDFTLTISSLQPEDFATYYCQQSYS | |
| | TPLTFGGGTKVEIKR | |
| 2 | DIVMTQSPLSLPVTPGEPASISCRSSQSLLH | 610 |
| | SNGYNYLDWYLQKPGQSPQLLIYLGSNRASG | |
| | VPDRFSGSGSGTDFTLKISRVEAEDVGVYYC | |
| | MQGLQTPHTFGQGTKVEIKR | |
| 3 | DIQMTQSPSSLSASVGDRVTITCRASQSISS | 611 |
| | YLNWYQQKPGKAPKLLIYAASSLQSGVPSRF | |
| | SGSGSGTDFTLTISSLQPEDFATYYCQQTYS | |
| | TPYTFGQGTKLEIKR | |
| 4 | DIQMTQSPSSLSASVGDRVTITCRASQTIGN | 612 |
| | YVNWYQQKPGKAPKLLIYGASNLHTGVPSRF | |
| | SGSGSGTDFTLTISSLQPEDFATYYCQQTYS | |
| | APLTFGGGTKVEIKR | |

### IV. Methods of Production Viral Vectors

Large scale viral vector production, e.g., lentiviral production, is often useful to achieve a desired viral titer. Viral vectors can be produced by transfecting a transfer vector into a packaging cell line that comprises viral structural and/or accessory genes, e.g., gag, pol, env, tat, rev, vif, vpr, vpu, vpx, or nef genes or other retroviral genes.

In some embodiments, viral vectors may be produced in multiple cell culture systems including bacteria, mammalian cell lines, insect cell lines, yeast and plant cells. Exemplary methods for producing viral vectors are described.

In some embodiments, elements for the production of a viral vector, e.g., a recombinant viral vector such as a replication incompetent lentiviral vector, are included in a packaging cell line or are present on a packaging vector. In some embodiments, viral vectors can include packaging elements, rev, gag, and pol, delivered to the packaging cells line via one or more packaging vectors.

In some embodiments, the packaging vector is an expression vector that lacks a packaging signal and comprises a polynucleotide encoding one, two, three, four or more viral structural and/or accessory genes. Typically, the packaging vectors are included in a packaging cell, and are introduced into the cell via transfection, transduction or infection. A lentiviral transfer plasmid can be introduced into a packaging cell line via transfection to generate a source cell or cell line. The packaging vectors can be introduced into human cells or cell lines by standard methods including, e.g., calcium phosphate transfection, lipofection or electroporation. In some embodiments, the packaging vectors are introduced into the cells together with a dominant selectable marker, such as neomycin, hygromycin, puromycin, blastocidin, zeocin, thymidine kinase, DHFR, Gln synthetase or ADA, followed by selection in the presence of the appropriate drug and isolation of clones. A selectable marker gene can be linked physically to genes encoding by the packaging vector, e.g., by IRES or self-cleaving viral peptides. In some embodiments, the packaging vector is a packaging plasmid.

Producer cell lines (also called packaging cell lines) include cell lines that do not contain a packaging signal, but do stably or transiently express viral structural proteins and replication enzymes (e.g., gag, pol and env) which can package viral vectors, e.g., lentiviral vectors. Any suitable cell line can be employed, e.g., mammalian cells, e.g., human cells. Suitable cell lines which can be used include, for example, CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211 A cells. In embodiments, the packaging cells are 293 cells, 293T cells, or A549 cells.

In some embodiments, a producer cell (i.e., a source cell line) includes a cell line which is capable of producing engineered lentiviral vectors, comprising a packaging cell line and a transfer plasmid construct comprising a packaging signal. Methods of preparing viral stock solutions are illustrated by, e.g., Y. Soneoka et al. (1995) Nucl. Acids Res. 23:628-633, and N. R. Landau et al. (1992) J. Virol. 66:5110-5113, which are incorporated herein by reference. Infectious virus vectors may be collected from the packaging cells, e.g., by cell lysis, or collection of the supernatant of the cell culture. Optionally, the collected virus vectors may be enriched or purified.

In some embodiments, the source cell comprises one or more plasmids coding for viral structural proteins and replication enzymes (e.g., gag, pol and env) which can package viral vectors (i.e., a packaging plasmid). In some embodiments, the sequences coding for at least two of the gag, pol, and env precursors are on the same plasmid. In some embodiments, the sequences coding for the gag, pol, and env precursors are on different plasmids. In some embodiments, the sequences coding for the gag, pol, and env precursors have the same expression signal, e.g., promoter. In some embodiments, the sequences coding for the gag, pol, and env precursors have a different expression signal, e.g., different promoters. In some embodiments, expression of the gag, pol, and env precursors is inducible. In some embodiments, the plasmids coding for viral structural proteins and replication enzymes are transfected at the same time or at different times. In some embodiments, the plasmids coding for viral structural proteins and replication enzymes are transfected at the same time or at a different time from the packaging vector.

In some embodiments, the source cell line comprises one or more stably integrated viral structural genes. In some embodiments expression of the stably integrated viral structural genes is inducible.

In some embodiments, expression of the viral structural genes is regulated at the transcriptional level. In some embodiments, expression of the viral structural genes is regulated at the translational level. In some embodiments, expression of the viral structural genes is regulated at the post-translational level.

In some embodiments, expression of the viral structural genes is regulated by a tetracycline (Tet)-dependent system, in which a Tet-regulated transcriptional repressor (Tet-R) binds to DNA sequences included in a promoter and represses transcription by steric hindrance (Yao et al, 1998; Jones et al, 2005). Upon addition of doxycycline (dox), Tet-R is released, allowing transcription. Multiple other suitable transcriptional regulatory promoters, transcription factors, and small molecule inducers are suitable to regulate transcription of viral structural genes.

In some embodiments, the third-generation lentivirus components, human immunodeficiency virus type 1 (HIV) Rev, Gag/Pol, and an envelope under the control of Tet-regulated promoters and coupled with antibiotic resistance cassettes are separately integrated into the source cell genome. In some embodiments the source cell only has one copy of each of Rev, Gag/Pol, and an envelope protein integrated into the genome.

In some embodiments, a lentiviral nucleic acid described herein is unable to undergo reverse transcription. Such a nucleic acid, in embodiments, is able to transiently express transgene. The engineered lentiviral vector may comprise a disabled reverse transcriptase protein, or may not comprise a reverse transcriptase protein. In embodiments, the lentiviral nucleic acid comprises a disabled primer binding site (PBS) and/or att site. In embodiments, one or more viral accessory genes, including rev, tat, vif, nef, vpr, vpu, vpx and S2 or functional equivalents thereof, are disabled or absent from the retroviral nucleic acid. In embodiments, one or more accessory genes selected from S2, rev and tat are disabled or absent from the lentiviral nucleic acid.

Typically, modern lentiviral vector systems include viral genomes bearing cis-acting vector sequences for transcription, reverse-transcription, integration, translation and packaging of viral RNA into the viral vectors, and (2) producer cells lines which express the trans-acting lentiviral gene sequences (e.g., gag, pol and env) needed for production of virus vectors. By separating the cis- and trans-acting vector sequences completely, the virus is unable to maintain replication for more than one cycle of infection. Generation of live virus can be avoided by a number of strategies, e.g., by minimizing the overlap between the cis- and trans-acting sequences to avoid recombination.

Generally, for viral vectors, expression of the gag precursor protein alone mediates vector assembly and release. In some aspects, gag proteins or fragments thereof have been demonstrated to assemble into structures analogous to viral cores. In one embodiment this may be achieved by using an endogenous packaging signal binding site on gag. Alternatively, the endogenous packaging signal binding site is on pol. In this embodiment, the RNA which is to be delivered will contain a cognate packaging signal. In another embodiment, a heterologous binding domain (which is heterologous to gag) located on the RNA to be delivered, and a cognate binding site located on gag or pol, can be used to ensure packaging of the RNA to be delivered. The heterologous sequence could be non-viral or it could be viral, in which case it may be derived from a different virus. The VLP could be used to deliver therapeutic RNA, in which case functional integrase and/or reverse transcriptase is not required. These VLPs could also be used to deliver a therapeutic gene of interest, in which case pol is typically included.

In an embodiment, gag-pol are altered, and the packaging signal is replaced with a corresponding packaging signal. In this embodiment, the viral vector can package the RNA with the new packaging signal. The advantage of this approach is that it is possible to package an RNA sequence which is devoid of viral sequence for example, RNAi.

In some embodiments, a polynucleotide comprises a nucleotide sequence encoding a viral gag protein or retroviral gag and pol proteins, wherein the gag protein or pol protein comprises a heterologous RNA binding domain capable of recognizing a corresponding sequence in an RNA sequence to facilitate packaging of the RNA sequence into a viral vector. In some embodiments, the heterologous RNA binding domain comprises an RNA binding domain derived from a bacteriophage coat protein, a Rev protein, a protein of the U 1 small nuclear ribonucleoprotein vector, a Nova protein, a TF 111 A protein, a TIS 11 protein, a trp RNA-binding attenuation protein (TRAP) or a pseudouridine synthase.

In some embodiments, formation of viral vectors can be detected by any suitable technique known in the art. Examples of such techniques include, e.g., electron microscopy, dynamic light scattering, selective chromatographic separation and/or density gradient centrifugation.

In some embodiments, methods described herein are methods of producing lentiviral vectors.

In some embodiments, a method described herein can comprise introducing an engineered lentiviral vector system comprising two or more transfer plasmid as described herein into producer cells. In some embodiments, the two or more transfer plasmids are present in different amounts. In some embodiments, the two or more transfer plasmids are present in the same amount. In some embodiments, the lentiviral vector system comprises at least a first transfer plasmid and a second transfer plasmid. In some embodiments, the first transfer plasmid and the second transfer plasmid are present at a ratio of between about 1:1 and 1:10 or between about 1:10 and 1:1. In some embodiments, the ratio of the first transfer plasmid and second transfer plasmid is about 1:1, about 1:2, about 1:3, about 1:4, about 1:5, about 1:6, about 1:7, about 1:8, about 1:9, about 1:10, about 2:1, about 3:1, about 4:1, about 5:1, about 6:1, about 7:1, about 8:1, about 9:1, about 10:1, about 2:3, about 3:2, about 2:5, about 3:5, about 5:2, or about 5:3.

In some embodiments producer cells comprise CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells.

In some embodiments, a method described herein can comprise culturing the producer cells under conditions sufficient to produce the viral vectors. In some embodiments, producer cells are cultured at 37° C. In some embodiments, producer cells are cultured at least at 37° C. In some embodiments, producer cells are cultured between 35° C. and 45° C. In some embodiments, producer cells are cultured while exposed to, among other gases, 5% carbon dioxide. In some embodiments, producer cells are cultured at a temperature that is permissive to viral vector production by a producer cell. In some embodiments, producer cells are cultured with one or more gas(es) that is/are permissive to viral vector production by a producer cell. In some embodiments, a producer cell culture is a suspension culture.

### V. Drug Substance

Methods provided herein are useful for the production of viral vectors. In some embodiments, such viral vectors will be utilized in a drug substance. A “drug substance” is an active ingredient (e.g., viral vectors) that is intended to furnish pharmacological activity or other direct effect in the diagnosis, cure, mitigation, treatment, or prevention of disease or to affect the structure or any function of a subject's body, but does not include intermediates used in the synthesis of such ingredient. A drug substance may need further processing to become a “drug product,” which is a finished dosage form (e.g., tablet or solution) to be administered to a subject. However, a drug substance does not require further processing to purify, isolate, or otherwise enrich the active ingredient prior to incorporation into a drug product.

Provided herein are methods of making a drug substance comprising a lentiviral vector with two or more genomes. In some embodiments, after producer cells are cultured under conditions sufficient to produce the viral vectors, a method comprises obtaining a subset of the producer cell culture comprising lentiviral vectors. In some embodiments, a subset of the producer cell culture comprises producer cells and/or culture medium.

In some embodiments, a method comprises enriching lentiviral vectors from the subset of the producer cell culture. In some embodiments, enriching lentiviral vectors form the subset of the producer cell culture comprises increasing the relative concentration of lentiviral vectors. In some embodiments, enriching lentiviral vectors form the subset of the producer cell culture comprises chromatographic and/or filtration methods.

In some embodiments, a method comprises removing producer cell DNA and/or producer cell protein from the subset of the producer cell culture. In some embodiments, removing producer cell DNA and/or producer cell protein from the subset of the producer cell culture comprises chromatographic and/or filtration methods.

In some embodiments, a method comprises adding a pharmaceutically acceptable excipient to enriched lentiviral vectors.

In some embodiments, a drug substance or drug product as described herein can include a pharmaceutically acceptable carrier or excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, MD, 2006; incorporated herein by reference) discloses various excipients used in formulating pharmaceutical compositions and known techniques for the preparation thereof. Suitable pharmaceutically acceptable carriers include but are not limited to water, salt solutions (e.g., NaCl), saline, buffered saline, glycerol, sugars such as mannitol, sucrose, or others, dextrose, fatty acid esters, etc., as well as combinations thereof.

A drug substance or drug product as described herein can, if desired, be mixed with auxiliary agents (e.g., lubricants, preservatives, stabilizers, wetting agents, emulsifiers, salts for influencing osmotic pressure, buffers, coloring, flavoring and/or aromatic substances and the like), which do not deleteriously react with the active compounds or interfere with their activity. In certain embodiments, a water-soluble carrier suitable for intravenous administration is used. In some embodiments, a pharmaceutical composition can be sterile.

A suitable drug substance or drug product as described herein, if desired, can also contain minor amounts of wetting or emulsifying agents, or pH buffering agents.

A drug product formulated from a drug substance described herein can be formulated in accordance with the routine procedures as a drug substance, e.g., adapted for administration to human beings. The formulation of a drug substance should suit the mode of administration. For example, in some embodiments, a drug substance for intravenous administration is typically a solution in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachet indicating the quantity of active agent. Where a drug substance is to be administered by infusion, it can be dispensed with an infusion bottle containing sterile pharmaceutical grade water, saline or dextrose/water. Where a drug substance is administered by injection, an ampule of sterile water for injection or saline can be provided so that the ingredients may be mixed prior to administration.

A drug product as described herein can be formulated for administration by any suitable route, such as, for example, intravenous, intratumoral, intraarterial, intramuscular, intraperitoneal, intrathecal, epidural, and/or subcutaneous administration routes. Preferably, the composition is formulated for a parenteral route of administration.

A composition suitable for parenteral administration can be an aqueous or nonaqueous, isotonic sterile injection solution, which can contain anti-oxidants, buffers, bacteriostats, and solutes, for example, that render the composition isotonic with the blood of the intended recipient. An aqueous or nonaqueous sterile suspension can contain one or more suspending agents, solubilizers, thickening agents, stabilizers, and preservatives.

Although the descriptions of drug substances provided herein are principally directed to drug substances that are suitable for ethical administration to humans, it will be understood by the skilled artisan that such compositions are generally suitable for administration to animals of all sorts or cells in vitro or ex vivo. Modification of drug substances suitable for administration to humans in order to render the compositions suitable for administration to various animals or cells in vitro or ex vivo is well understood, and the ordinarily skilled practitioner, e.g., a veterinary pharmacologist, can design and/or perform such modification with merely ordinary, if any, experimentation.

Formulations of drug substances described herein may be prepared by any method known or hereafter developed in the art of pharmacology. In general, such preparatory methods include the step of bringing the active ingredient into association with a diluent or another excipient and/or one or more other accessory ingredients, and then, if necessary and/or desirable, shaping and/or packaging the product into a desired single- or multi-dose unit.

### VII. Methods of Use

In some embodiments, viral vectors comprising two or more genomes (e.g., payload genes) provided herein or pharmaceutical compositions containing same can be administered to a subject, e.g. a mammal, e.g. a human. In some embodiments, the viral vectors and payload genes are administered by the provided system of ex vivo dosing and administration. In some embodiments, the methods and uses involve dosing the therapy in-line in a closed fluid circuit attached or operably connected to the subject being treated. In some embodiments, the fluid pathway from the whole blood sample from the subject to the reinfusion of the transduction mixture containing the viral vector and PBMCs or subset, such as a leukapheresis or apheresis cell composition, is closed so that the entire process occurs while the system is connected to the subject or patient.

In such embodiments, the subject may be at risk of, may have a symptom of, or may be diagnosed with or identified as having, a particular disease or condition. In one embodiment, the subject has cancer. In one embodiment, the subject has an infectious disease. In some embodiments, the viral vectors, such as a targeted viral vector, contains nucleic acid sequences encoding the payload agent (also interchangeably called an exogenous agent or in some cases “cargo”) for treating the disease or condition in the subject. Thus, in some embodiments, the disease or condition that is treated is any that may be treatable by the encoded payload agent. For instance, in some embodiments the payload agent encodes a chimeric antigen receptor (CAR) that specifically binds to an antigen, and the disease or condition to be treated can be any in which expression of the antigen is associated with and/or involved in the etiology of a disease condition or disorder, e.g. causes, exacerbates or otherwise is involved in such disease, condition, or disorder. Exemplary diseases and conditions can include diseases or conditions associated with malignancy or transformation of cells (e.g. cancer), autoimmune or inflammatory disease, or an infectious disease, e.g. caused by bacterial, viral or other pathogens. Exemplary antigens, which include antigens associated with various diseases and conditions that can be treated, include any of antigens described herein.

For example, the exogenous agent is one that targets or is specific for a protein of a neoplastic cells and the viral vector is administered to a subject for treating a tumor or cancer in the subject. In another example, the exogenous agent is an inflammatory mediator or immune molecule, such as a cytokine, and viral vector is administered to a subject for treating any condition in which it is desired to modulate (e.g. increase) the immune response, such as a cancer or infectious disease. In some embodiments, the viral vector is administered in an effective amount or dose to effect treatment of the disease, condition, or disorder.

Provided herein are uses of any of the provided viral vectors in such methods and treatments, and in the preparation of a medicament in order to carry out such therapeutic methods. In some embodiments, the methods are carried out by administering the viral vector or compositions comprising the same, to the subject having, having had, or suspected of having the disease or condition or disorder. In some embodiments, the methods thereby treat the disease or condition or disorder in the subject. Also provided herein are uses of any of the compositions, such as pharmaceutical compositions provided herein, for the treatment of a disease, condition or disorder associated with a particular gene or protein targeted by or provided by the exogenous agent.

In some embodiments, the viral vector or compositions described herein can be administered to a subject, e.g., a mammal, e.g., a human. In some of any embodiments, the subject may be at risk of, may have a symptom of, or may be diagnosed with or identified as having, a particular disease or condition (e.g., a disease or condition described herein). In some embodiments, the disease is a disease or disorder.

In some embodiments, the transgene/payload agent is a chimeric antigen receptor, including any as described in Section II.A and/or that specifically binds an antigen described in Section II.A. Exemplary target antigens include, but are not limited to, CD5, CD19, CD20, CD22, CD23, CD30, CD70, Kappa, Lambda, and B cell maturation agent (BCMA), G-protein coupled receptor family C group 5 member D (GPRC5D) (associated with leukemias); CS1/SLAMF7, CD38, CD138, GPRC5D, TACI, and BCMA (associated with myelomas); GD2, HER2, EGFR, EGFRvIII, B7H3, PSMA, PSCA, CAIX, CD171, CEA, CSPG4, EPHA2, FAP, FRα, IL-13Ra, Mesothelin, MUC1, MUC16, and ROR1 (associated with solid tumors). In some embodiments, the first transgene encodes a first CAR and the second transgene encodes a second CAR, wherein the first CAR and the second CAR include different antigen binding domains that target different antigens expressed on a target cell. In some embodiments, the first CAR is a CD19 CAR and the second CAR is a CD22 CAR; the first CAR is a B cell maturation agent (BCMA) CAR and the second CAR is a G-protein coupled receptor family C group 5 member D (GPRC5D) CAR; the first CAR is a CD19 CAR and the second CAR is a CD20 CAR; the first CAR is a CD20 CAR and the second CAR is a CD22 CAR; or the first CAR is a CD19 CAR and the second CAR is a BCMA CAR; the first CAR is a CD20 CAR and the second CAR is a L1-CAM CAR; the first CAR is a L1-CAM CAR and the second CAR is a GD2 CAR; the first CAR is a EGFR CAR and the second CAR is a L1-CAM CAR; the first CAR is a EGFR CAR and the second CAR is a C-MET CAR; the first CAR is a EGFR CAR and the second CAR is a HER2 CAR; the first CAR is a C-MET CAR and the second CAR is a HER2 CAR; or the first CAR is a EGFR CAR and the second CAR is a ROR1 CAR.

In some embodiments, the disease or condition is a B cell malignancy and the antigen targeted by the CAR is expressed by cells associated with the B cell malignancy. In some embodiments, the antigen is CD19. In some embodiments, the antigen is CD20. In some embodiments, the antigen is CD22. In some embodiments, the antigen is BCMA. In some embodiments, the B cell malignancy is a Large B-cell Lymphoma (LBCL). In some embodiments, the disease or condition has relapsed or the subject is refractory to treatment of the disease or condition. For instance, in some embodiments, the disease or condition is relapsed and/or refractory Large B-cell Lymphoma (LBCL). In some embodiments, LBCL include Non-Hodgkin's lymphoma, including diffuse large B-cell lymphoma (DLBCL) not otherwise specified (including DLBCL arising from indolent lymphoma), primary mediastinal large B-cell lymphoma, high grade B-cell lymphoma, follicular lymphoma, and marginal zone lymphoma. In some embodiments, the subject has received or is receiving prior to the ex vivo dosing provided herein prior therapies, such as two or more lines of systemic therapy for treating the disease or condition. In some embodiments, the subject has relapsed and/or is refractory to the prior therapies. In some embodiments, the prior therapies include two or more prior therapies from a chemotherapy containing regimen, such as with anthracycline, or an anti-CD20 mAb (unless CD20 negative), or after autologous stem cell transplant (ASCT). In some embodiments, the subject has one or more measurable PET-positive lesion, such as measured per Lugano classification. In some embodiments, the subject as an ECOG performance status of 0 or 1. In some embodiments, the subject has adequate organ function.

In some embodiments, the disease or condition is a multiple myeloma and the antigen targeted by the CAR is expressed by cells associated with the multiple myeloma. In some embodiments, the antigen is BCMA. In some embodiments, the subject has or is suspected of having a multiple myeloma that is associated with expression of B cell maturation antigen (BCMA). In some embodiments, the multiple myeloma is a relapsed and/or refractory multiple myeloma.

In some aspects, response rates in subjects, such as subjects with LBCL, are based on the Lugano criteria. (Cheson et al., (2014) JCO., 32(27):3059-3067; Johnson et al., (2015) Radiology 2:323-338; Cheson, B. D. (2015) Chin. Clin. Oncol. 4(1):5). In some aspects, response assessment utilizes any of clinical, hematologic, and/or molecular methods. In some aspects, response assessed using the Lugano criteria involves the use of positron emission tomography (PET)-computed tomography (CT) and/or CT as appropriate. PET-CT evaluations may further comprise the use of fluorodeoxyglucose (FDG) for FDG-avid lymphomas. In some aspects, where PET-CT will be used to assess response in FDG-avid histologies, a 5-point scale may be used. In some respects, the 5-point scale comprises the following criteria: 1, no uptake above background; 2, uptake≤mediastinum; 3, uptake>mediastinum but≤liver; 4, uptake moderately>liver; 5, uptake markedly higher than liver and/or new lesions; X, new areas of uptake unlikely to be related to lymphoma.

In some embodiments, response is based on lack of detectable minimal residual disease (MRD negativity) which means that no disease is detected. Methods for assessing MRD include, but are not limited to flow cytometry, polymerase chain reaction (PCR) and next-generation sequencing. In some embodiments, a sample of bone marrow cells and/or peripheral blood cells is assessed for disease. For instance, certain mutations or genetic abnormalities can be assessed that are known to be associated with the cancer. A skilled artisan is familiar with methods to assess MRD.

In some cases, the pharmacokinetics of cells expressing the payload agent (e.g. CAR) are determined to assess the bioavailability of the engineered cells in vivo. Methods for determining the pharmacokinetics of engineered cells in vivo may include drawing peripheral blood from subjects that have received the ex vivo dosing and determining the number of engineered cells in the blood based on detection of the engineered payload agent (CAR) expressed by the cells. For example, an anti-idiotypic antibody against the CAR may be used for detection of the CAR-expressing cells.

In some embodiments, the regimen of administration may affect what constitutes an effective amount. In some embodiments, the therapeutic formulations may be administered to the subject either prior to or after a diagnosis of disease. In some embodiments, several divided dosages, as well as staggered dosages may be administered daily or sequentially, or the dose may be continuously infused, or may be a bolus injection. In some embodiments, the dosages of the therapeutic formulations may be proportionally increased or decreased as indicated by the exigencies of the therapeutic or prophylactic situation.

In some embodiments, an effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the subject being treated, and like factors well-known in the medical arts. In some embodiments, the dosage regimens may be adjusted to provide the optimum therapeutic response. In some embodiments, several divided doses may be administered daily or the dose may be proportionally reduced as indicated by the exigencies of the therapeutic situation. One of ordinary skill in the art would be able to study the relevant factors and make the determination regarding the effective amount of the therapeutic compound without undue experimentation.

A medical doctor, e.g., physician or veterinarian, having ordinary skill in the art may readily determine and prescribe the effective amount of the pharmaceutical composition required. In some embodiments, the physician or veterinarian could start doses of the compounds of the invention employed in the pharmaceutical composition at levels lower than that required in order to achieve the desired therapeutic effect and gradually increase the dosage until the desired effect is achieved.

In some embodiments, routes of administration of any of the compositions disclosed herein include oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, and topical administration. In some embodiments, the administration is by infusion such as by intravenous infusion.

In some embodiments, the provided embodiments do not involve a lymphodepletion therapy prior to the ex vivo administration of the viral vector. Thus, the provided methods do not involve administration of lymphopleting regimens, such as those including cyclophosphamide and/or fludarabine and/or bendamustine, or other lymphodepleting regimens or protocols, prior to receiving administration of the viral vector. It is understood that the exclusion of lymphodepleting therapies in accord to the provided methods does not exclude that the subject may have previous in time (e.g. months to years earlier) may have received a lymphodepleting therapy. Rather, the provided embodiments include those in which the subject has not in accord with the present dosing methods received a lymphodepleting therapy, such as within 60 days or 30 days, prior to the ex vivo administration of the viral vector of the present methods.

In some embodiments, the viral vector composition comprising an exogenous agent or cargo, may be used to deliver such exogenous agent or cargo to a cell tissue or subject. In some embodiments, delivery of a cargo by administration of a viral vector composition described herein may modify cellular protein expression levels. In certain embodiments, the administered composition directs upregulation of (via expression in the cell, delivery in the cell, or induction within the cell) of one or more cargo (e.g., a polypeptide or mRNA) that provide a functional activity which is substantially absent or reduced in the cell in which the polypeptide is delivered. In some embodiments, the missing functional activity may be enzymatic, structural, or regulatory in nature. In some embodiments, the administered composition directs up-regulation of one or more polypeptides that increases (e.g., synergistically) a functional activity which is present but substantially deficient in the cell in which the polypeptide is upregulated. In some of any embodiments, the administered composition directs downregulation of (via expression in the cell, delivery in the cell, or induction within the cell) of one or more cargo (e.g., a polypeptide, siRNA, or miRNA) that repress a functional activity which is present or upregulated in the cell in which the polypeptide, siRNA, or miRNA is delivered. In some of any embodiments, the upregulated functional activity may be enzymatic, structural, or regulatory in nature. In some embodiments, the administered composition directs down-regulation of one or more polypeptides that decreases (e.g., synergistically) a functional activity which is present or upregulated in the cell in which the polypeptide is downregulated. In some embodiments, the administered composition directs upregulation of certain functional activities and downregulation of other functional activities.

In some of any embodiments, the viral vector composition (e.g., one comprising mitochondria or DNA) mediates an effect on a target cell, and the effect lasts for at least 1, 2, 3, 4, 5, 6, or 7 days, 2, 3, or 4 weeks, or 1, 2, 3, 6, or 12 months. In some embodiments (e.g., wherein the viral vector composition comprises an exogenous protein), the effect lasts for less than 1, 2, 3, 4, 5, 6, or 7 days, 2, 3, or 4 weeks, or 1, 2, 3, 6, or 12 months.

In some of any embodiments, the viral vector composition described herein is delivered for ex-vivo administration to a cell or tissue, e.g., a human cell or tissue. In embodiments, the composition improves function of a cell or tissue ex-vivo, e.g., improves cell viability, respiration, or other function (e.g., another function described herein).

In some embodiments, the composition is delivered for ex vivo administration to a tissue that is in an injured state (e.g., from trauma, disease, hypoxia, ischemia or other damage).

In some embodiments, the composition is delivered for ex-vivo transplant (e.g., a tissue explant or tissue for transplantation, e.g., a human vein, a musculoskeletal graft such as bone or tendon, cornea, skin, heart valves, nerves; or an isolated or cultured organ, e.g., an organ to be transplanted into a human, e.g., a human heart, liver, lung, kidney, pancreas, intestine, thymus, eye). In some embodiments, the composition is delivered to the tissue or organ before, during and/or after transplantation.

In some embodiments, the viral vector compositions described herein can be administered to a subject, e.g., a mammal, e.g., a human. In such embodiments, the subject may be at risk of, may have a symptom of, or may be diagnosed with or identified as having, a particular disease or condition (e.g., a disease or condition described herein).

In some embodiments, the source of viral vector are from the same subject that is administered a viral vector composition. In other embodiments, they are different. In some embodiments, the source of viral vector and recipient tissue may be autologous (from the same subject) or heterologous (from different subjects). In some embodiments, the donor tissue for viral vector compositions described herein may be a different tissue type than the recipient tissue. In some embodiments, the donor tissue may be muscular tissue and the recipient tissue may be connective tissue (e.g., adipose tissue). In other embodiments, the donor tissue and recipient tissue may be of the same or different type, but from different organ systems.

In some embodiments, the viral vector composition described herein may be administered to a subject having a cancer, an autoimmune disease, an infectious disease, a metabolic disease, a neurodegenerative disease, or a genetic disease (e.g., enzyme deficiency). In some embodiments, the subject is in need of regeneration.

In some embodiments, the viral vector is co-administered with an inhibitor of a protein that inhibits membrane fusion. For example, Suppressyn is a human protein that inhibits cell-cell fusion (Sugimoto et al., “A novel human endogenous retroviral protein inhibits cell-cell fusion” Scientific Reports 3: 1462 (DOI: 10.1038/srep01462)). In some embodiments, the viral vector vectors is co-administered with an inhibitor of sypressyn, e.g., a siRNA or inhibitory antibody.

In some embodiment, described here in, method of producing a CAR-T cell by transducing a T cell with the lentiviral vector of the present disclosure by contacting the T cell with the lentiviral vector, wherein the lentiviral vector comprises a first genome comprising a first transgene; and a second genome comprising a second transgene; and the first transgene encodes a CAR and the second transgene encodes a second CAR, wherein the first CAR and the second CAR comprise different antigen binding domains that target different antigens expressed on a target cell. In some embodiments, the contacting is in vitro or ex vivo. In some embodiments, the CAR-T cell expresses a first CAR and a second CAR, wherein: the first CAR is a CD19 CAR and the second CAR is a CD22 CAR; the first CAR is a B cell maturation agent (BCMA) CAR and the second CAR is a G-protein coupled receptor family C group 5 member D (GPRC5D) CAR; the first CAR is a CD19 CAR and the second CAR is a CD20 CAR; the first CAR is a CD20 CAR and the second CAR is a CD22 CAR; or the first CAR is a CD19 CAR and the second CAR is a BCMA CAR; the first CAR is a CD20 CAR and the second CAR is a L1-CAM CAR; the first CAR is a L1-CAM CAR and the second CAR is a GD2 CAR; the first CAR is a EGFR CAR and the second CAR is a L1-CAM CAR; the first CAR is a EGFR CAR and the second CAR is a C-MET CAR; the first CAR is a EGFR CAR and the second CAR is a HER2 CAR; the first CAR is a C-MET CAR and the second CAR is a HER2 CAR; or the first CAR is a EGFR CAR and the second CAR is a ROR1 CAR.

In some embodiments, the first CAR and the second CAR are expressed by the cell at a ratio of between about 1:1 and 1:10 or between about 1:10 and 1:1. In some embodiments, the ratio of the first CAR and second CAR is about 1:1, about 1:2, about 1:3, about 1:4, about 1:5, about 1:6, about 1:7, about 1:8, about 1:9, about 1:10, about 2:1, about 3:1, about 4:1, about 5:1, about 6:1, about 7:1, about 8:1, about 9:1, about 10:1, about 2:3, about 3:2, about 2:5, about 3:5, about 5:2, or about 5:3.

In some embodiments the CAR-T cells are hypoimmunogenic. For example, the CAR-T cells comprise further genetic modification produce a CAR-T cell less prone to immune rejection by a subject into which such cell is transplanted. For example, relative to an unaltered or unmodified wild-type cell, such a hypoimmunogenic cell is about 2.5%, 5%, 10%, 20%, 30%, 40%, 50%, 60%, 70%, 80%, 90%, 95%, 97.5%, 99% or less prone to immune rejection by a subject into which such cell is transplanted. In some examples described herein, genome editing technologies are used to reduce the expression of MHC I and/or MHC II genes, and thus, to generate a hypoimmunogenic cell. In other examples described herein, a tolerogenic factor is introduced into a T cell and when expressed can reduce the ability of the cell to be recognized by host immune system and thus confer hypoimmunogenicity. Hypoimmunogenicity of a cell is determined by evaluating the cell's ability to elicit adaptive and innate immune responses. Such immune response can be measured using assays recognized by those skilled in the art, for example, by measuring the effect of a hypoimmunogenic cell on T cell proliferation, T cell activation, T cell killing, NK cell proliferation, NK cell activation, and macrophage activity. Hypoimmunogenic cells may undergo decreased killing by T cells and/or NK cells upon administration to a subject or show decreased macrophage engulfment compared to an unmodified or wildtype cell. In some embodiments, a hypoimmunogenic cell elicits a reduced or diminished immune response in a recipient subject compared to a corresponding unmodified wild-type cell. In some embodiments, a hypoimmunogenic cell is nonimmunogenic or fails to elicit an immune response in a recipient subject.

In some embodiments, described herein, a method of treating a disease or condition in a subject comprising administering the CAR-T cell expressing first CAR and the second CAR to a subject in need thereof. In some embodiments, the disease or condition is a cancer. In some embodiments, the disease or condition is a B cell acute lymphoblastic leukemia (B-ALL), diffuse large B-cell lymphoma, acute myeloid lymphoid leukemia, or multiple myeloma. In some embodiments, the cancer is B cell acute lymphoblastic leukemia (B-ALL), diffuse large B-cell lymphoma, liver cancer, pancreatic cancer, breast cancer, ovarian cancer, colorectal cancer, lung cancer, non-small cell lung cancer, acute myeloid lymphoid leukemia, multiple myeloma, gastric cancer, gastric adenocarcinoma, pancreatic adenocarcinoma, glioblastoma, neuroblastoma, lung squamous cell carcinoma, hepatocellular carcinoma, and bladder cancer.

In some embodiments, relapse of the disease or condition is prevented or delayed compared to a subject diagnosed with relapse of the disease or condition that was treated with a CAR-T cell targeting a single antigenic target. In some embodiments, the single antigenic target is CD19, CD20, CD22, or BCMA.

The disclosure is further illustrated by the following examples. The examples are provided for illustrative purposes only. They are not to be construed as limiting the scope or content of the disclosure in any way.

### EXAMPLES

### Example 1: Transduction of Cells with Lentiviral Vectors Having Two Genomes

This Example describes the production of lentiviral vectors that include two genomes (e.g., transfer plasmids) and compares the production with that of lentiviral vectors that include a single genome. This Example further describes transduction of primary T cells with the produced lentiviral vectors and provides data evidencing expression of proteins encoded by each of the two genomes (e.g., transfer plasmids) packaged within the lentiviral vectors. This Example provides a comparison of expression of proteins encoded by each of the two genomes (e.g., transfer plasmids) packaged within two different lentiviral vectors or packaged within a single lentiviral vector (vCAR).

Cell cultures were seeded in T225 flasks and cultured for a day. The cell cultures were then each transfected with a transfection mixture that included a lentiviral vector system for the production of lentiviral vectors.

Lentiviral vectors including a single genome were generated. Some of the single-genome lentiviral vectors comprised a single genome that encoded a CD19 CAR; other of the single-genome lentiviral vectors comprised a single genome that encoded a CD22 CAR. To generate lentiviral vectors including a single genome, the lentiviral vector system included a packaging plasmid, an envelope plasmid, and a transfer plasmid, wherein the transfer plasmid comprised either a transgene encoding a CD19 CAR or a transgene encoding a CD22 CAR.

Lentiviral vectors including two genomes were also generated (vCAR). For lentiviral vectors including two genomes, the lentiviral vector system included a packaging plasmid, an envelope plasmid, and two transfer plasmids (each having a transgene encoding either a CD19 or CD22 CAR). In other words, the lentiviral vector system included a first transfer plasmid including a transgene encoding a CD19-specific CAR and a second transfer plasmid encoding a CD22-specific CAR.

Following transfection, the producer cell cultures were further cultured for two days to allow for the production of lentiviral vectors. Supernatant comprising the produced lentiviral vectors was then collected from each of the cultures and enriched (e.g., using chromatographic and filtration) to obtain concentrated lentiviral vector compositions (Two LV Composition and vCAR Composition).

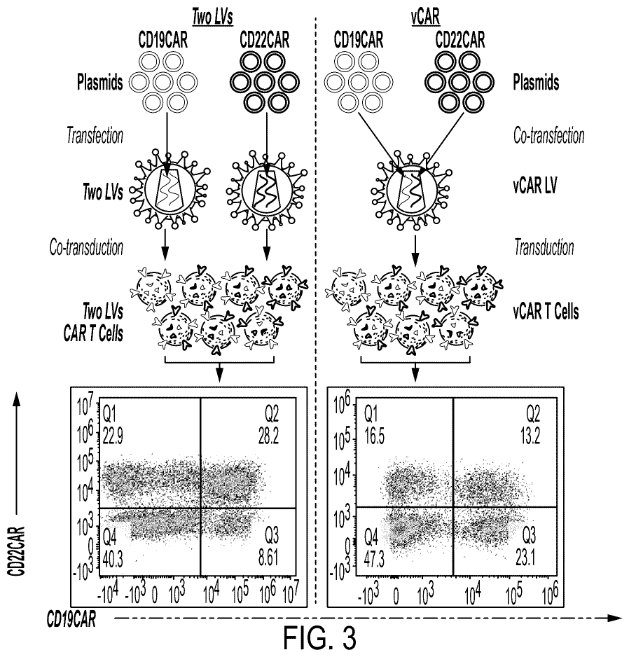

Primary T cells were then transduced with the Two LV Composition or the vCAR Composition in triplicate. After 6 days, the cells were collected and stained for flow cytometry. The cells were analyzed for CD19 CAR and CD22 CAR expression. As shown in FIG. 3, the cells transduced with both the Two LV Composition and the vCAR Composition were able to doubly express both CD 19 CAR and CD22 CAR.

The data provided herein demonstrates that proteins encoded on two genomes in a lentiviral vector can be expressed following delivery of the genomes to a cell. The data also shows that lentiviral vectors including two genomes can be used as alternative to a mixture of lentiviral vectors that each include a single genome.

### Example 2: Transduction of Cells with Lentiviral Vectors Having Two Genomes

This Example describes the production of lentiviral vectors that include two genomes (e.g., transfer plasmids). This Example further describes transduction of primary T cells with the produced lentiviral vectors and provides data evidencing expression of proteins encoded by each of the two genomes (e.g., transfer plasmids) packaged within the lentiviral vectors.

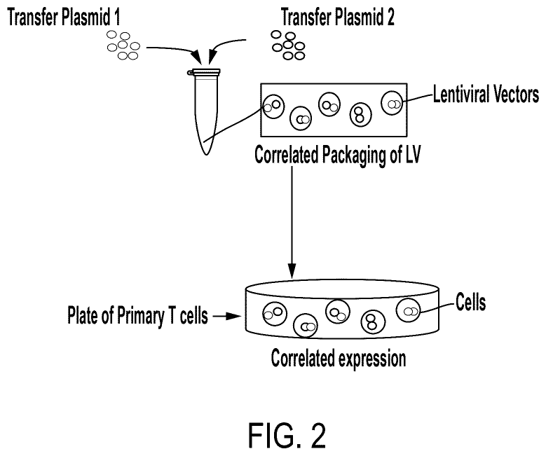

Three cell cultures were seeded in T225 flasks and cultured for a day. The three cell cultures were then each transfected with a transfection mixture that included a lentiviral vector system for the production of lentiviral vectors. The lentiviral vector system included a packaging plasmid, an envelope plasmid, and two transfer plasmids. See, e.g., FIG. 2. The first transfer plasmid (Transfer Plasmid 1) encoded CD47 protein and a CD19CAR. The second transfer plasmid (Transfer Plasmid 2) encoded CD47 protein and a CD22CAR. The transfection mixture for each of the three cell cultures included different ratios of Transfer Plasmid 1 and Transfer Plasmid 2, as shown below:

| Producer Cell | Transfection |
|---|---|
| Culture | Mixture Composition |
| Culture 1 | 25% Transfer Plasmid 1/ |
| | 75% Transfer Plasmid 2 |
| Culture 2 | 50% Transfer Plasmid 1/ |
| | 50% Transfer Plasmid 2 |
| Culture 3 | 75% Transfer Plasmid 1/ |
| | 25% Transfer Plasmid 2 |

Following transfection, the three producer cell cultures were further cultured for two days to allow for the production of lentiviral vectors. Supernatant comprising the produced lentiviral vectors was then collected from each of the cultures and enriched (e.g., using chromatographic and filtration) to obtain concentrated lentiviral vector compositions (Lentiviral Composition 1, Lentiviral Composition 2, and Lentiviral Composition 3).

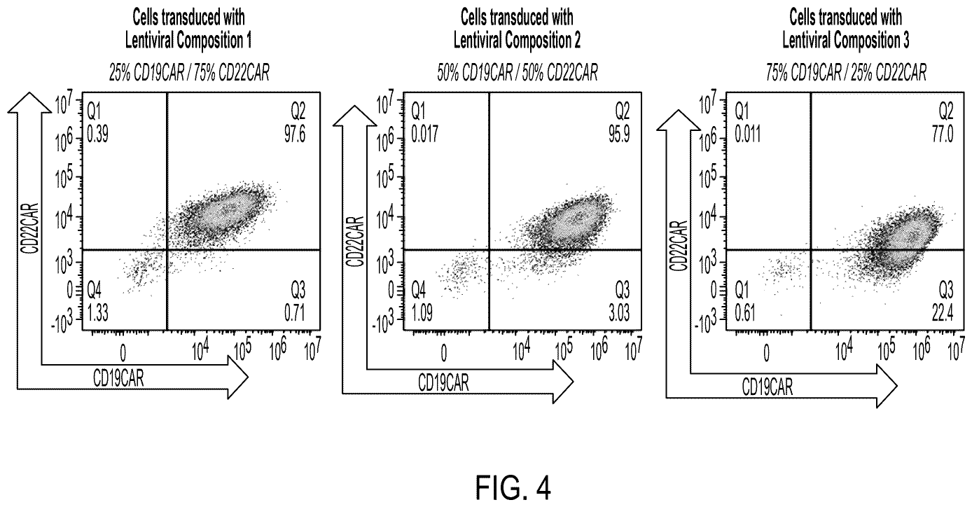

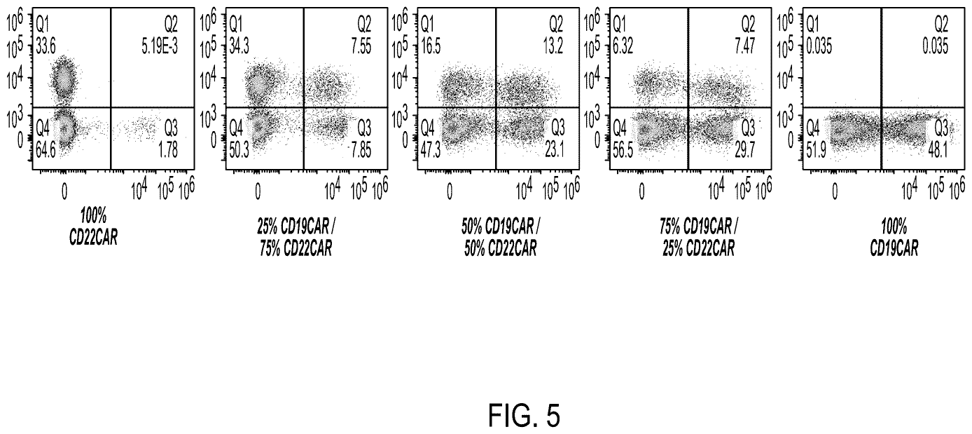

Primary T cells were then transduced with Lentiviral Composition 1, Lentiviral Composition 2, or Lentiviral Composition 3 in triplicate. After 72 hours, half the cells in each culture were taken out for vector copy number analysis (data not shown). Following an additional 72 hours, the remaining cells were collected and stained for flow cytometry. The cells were analyzed for CD19 CAR and CD22 CAR expression. As shown in FIG. 4, the transduced cells were able to doubly express both CD19 CAR and CD22 CAR. Further, the ratio of Transfer Plasmid 1 and Transfer Plasmid 2 within the lentiviral vectors used for transduction influenced the expression levels of encoded CD19 CAR and CD22 CAR from the transduced cells. For example, as the ratio of Transfer Plasmid 1 (encoding CD19 CAR) to Transfer Plasmid 2 (encoding CD22 CAR) in the transfection mixture (and in turn, the Lentiviral Composition) increases, the amount of CD19 CAR expressed also increases, as shown by the shift in the cell populations to the right. Similarly, as the ratio of Transfer Plasmid 1 (encoding CD19 CAR) to Transfer Plasmid 2 (encoding CD22 CAR) in the transfection mixture (and in turn, the Lentiviral Composition) increases, the amount of CD22 CAR expressed decreases, as shown by the shift in the cell populations to toward the bottom of the graphs.

The data provided herein demonstrates that proteins encoded on two genomes in a lentiviral vector can be expressed following delivery of the genomes to a cell. The data further demonstrates that expression ratios of multiple proteins encoded by two genomes in a lentiviral vector can be adjusted by adjusting the ratio of the two genomes within the lentiviral vector.

This data that, by tuning the ratios of the two transfer plasmids, the expression of proteins encoding on transfer plasmids can be skewed in cells, e.g., favoring one protein over another. Importantly, the tuned co-transfection method described in this Example allows the generation of these cells expressing multiple transgenes from a single heterogeneous lentiviral vector pool following a single transduction step. This simple method has the potential to overcome biological, CMC, safety and limitations of other approaches used to generate therapeutic cells with multiple transgenes.

### Example 3: Effective Control of Tumor Cells by CAR-T Cells Generated Using Lentiviral Vectors Having Two Genomes

This Example demonstrates the ex vivo and in vivo efficacy of lentiviral vectors that include two genomes for expressing transgenes. Further, this Example describes use of lentiviral vectors (e.g., as produced in Example 2) expressing CD19 CAR, CD22 CAR, or both for the control and killing of cancer cells.

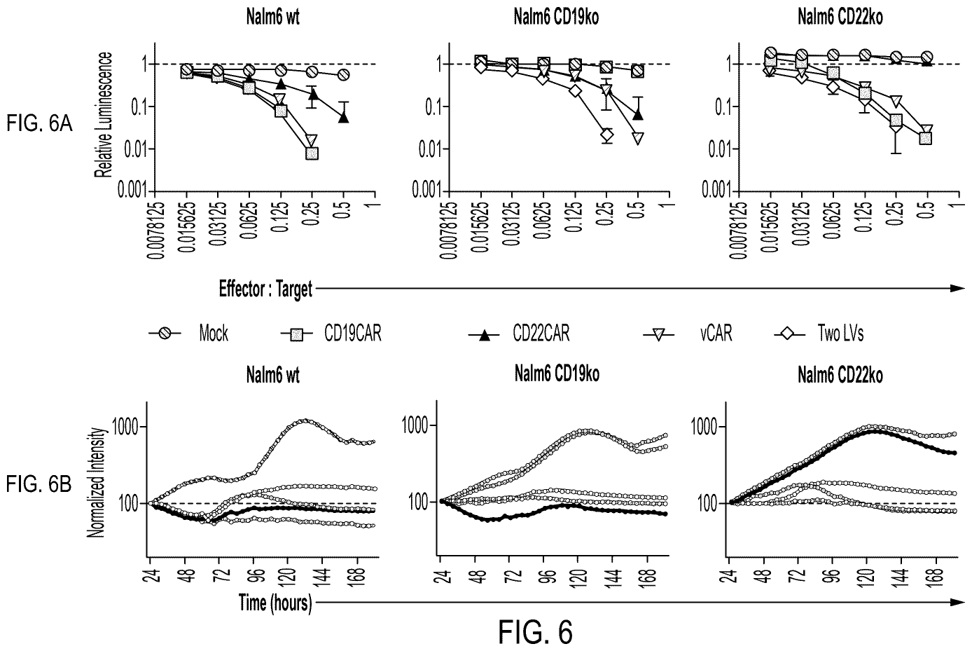

Nalm6 (acute lymphoblastic leukemia) wild-type cells, Nalm6 CD19 knockout (KO) cells, and Nalm6 CD22 KO cells were cultured and challenged with CAR T-cells expressing a CD19 CAR, a CD22 CAR, or both CD19 and CD22 CARs. The CAR T-cells expressing both CD19 and CD22 CARs were generated either as described herein (“vCAR”) or by transducing T cells with two separate lentiviral vectors, one with a genome encoding CD19 CAR and the other with a genome encoding CD22 CAR (“Two LVs”). See, e.g., FIG. 3. As shown in FIG. 6, CAR-T cells expressing both CD19 and CD22 CARs from a single heterogeneous lentiviral vector effectively kill Nalm6 cells in a dose dependent (FIG. 6A, Luciferase Cytotoxicity assay) and time dependent manner (FIG. 6B). Also, as expected, CAR T-cells expressing a CD19 CAR did not kill Nalm6 CD19 KO cells, and CAR T-cells expressing a CD22 CAR did not kill Nalm6 CD22 KO cells. Further, the results observed with vCAR generated T-cells were similar to the results observed with Two LV generated T-cells.

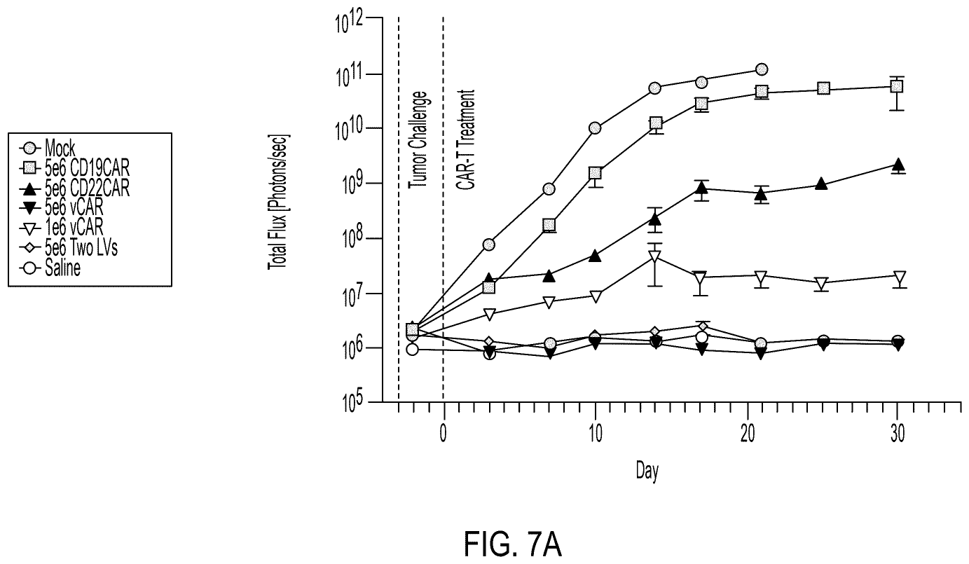

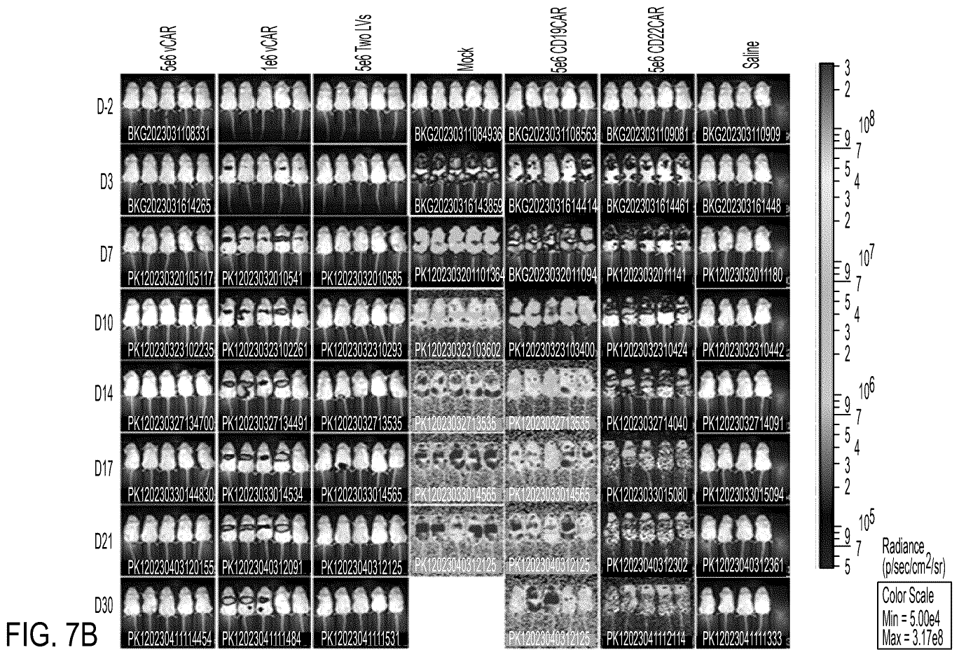

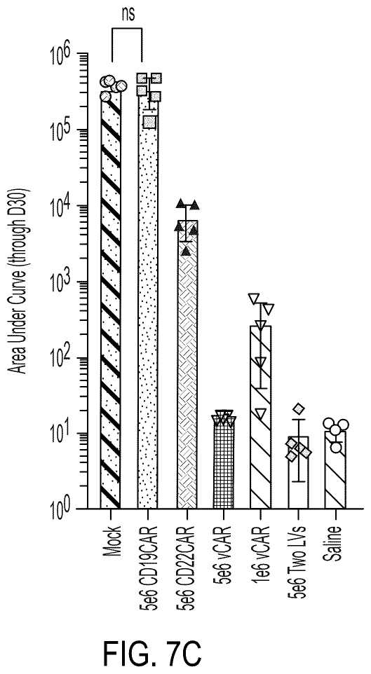

To assess whether CAR-T cells expressing both CD19CAR and CD22CAR from a single heterogeneous lentiviral vector would be effective in killing cancer cells in vivo, the CAR-T cells generated using vCAR lentiviral vectors were tested in mice. Immunodeficient NSG™ mice were challenged intravenously with a population of Nalm6 cells. 50% of the population were CD19KO and 50% of the population were CD22KO. Four days after tumor challenge the mice were treated with CAR-T cells expressing a CD19 CAR (5×10⁶ cells), a CD22 CAR (5×10⁶ cells), or both CD19 and CD22 CARs generated either by either transducing T cells with two separate lentiviral vectors (“Two LVs”, 5×10⁶ cells) or lentiviral vectors made using the two vector approach as described herein (“vCAR”, 1×10⁶ cells, and 5×10⁶ cells). As shown in FIG. 7, CAR-T cells expressing both CD19 and CD22 CARs from a heterogeneous lentiviral vector (vCAR) effectively controlled the growth of Nalm6 tumors in vivo.

### Example 4: CAR-T Cells Produced By Lentiviral Vectors Having Two Genomes Control Tumor Cells in a Model of Antigen Escape

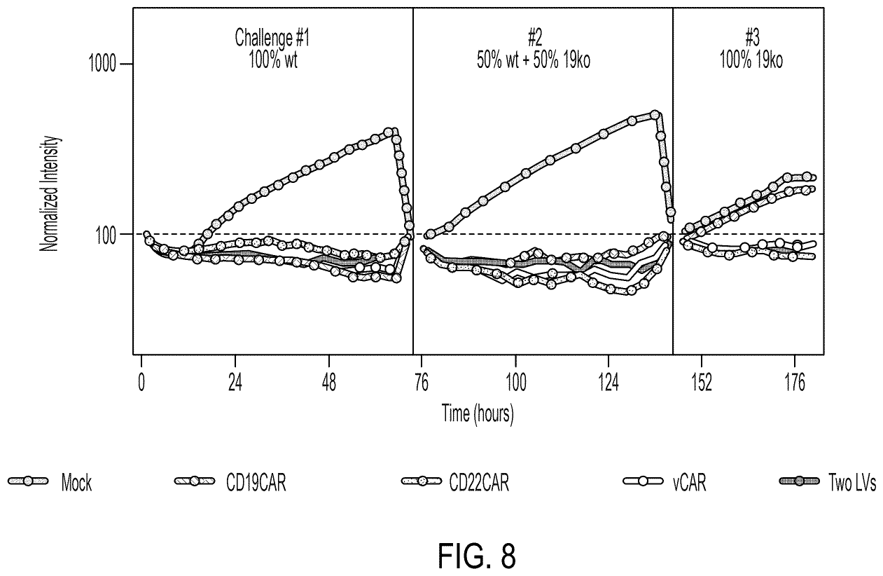

This Example demonstrates that CAR-T cells generated using lentiviral vectors described herein are able to kill tumor cells in vivo after antigen escape. Mice were challenged with a population of wild-type Nalm6 cells (“Challenge 1”), a population of 50% wild type Nalm6 cells and 50% CD19KO (“Challenge 2”), or a population of CD19KO Nalm6 cells (“Challenge 3”). CAR-T cells expressing a CD19 CAR, a CD22 CAR, or both CD19 and CD22 CARs were used to treat the mice. The CAR-T cells expressing both CD19 and CD22 CARs were generated either by transducing T cells with two separate lentiviral vectors (“Two LVs”) or lentiviral vectors as described herein (“vCAR”). FIG. 8 shows that in each condition the CAR-T cells generated using lentiviral vectors using the two vector approach as described herein are able to address antigen escape through dual CAR expression. In other words, FIG. 8 shows that in each condition the CAR-T cells generated using Two LVs or vCAR were able to address antigen escape through dual CAR expression.

### Example 5: Transduction of Cells with Lentiviral Vectors Having Two Genomes to Generate Car-T Cells Expressing a Bcma-Specific Car and a Gpc5D-Specific Car

This Example describes the production of lentiviral vectors that include two genomes (e.g., transfer plasmids). The first genome includes a transgene encoding a BCMA-specific CAR. The second genome includes a transgene encoding a GPC5D-specific CAR. This Example further describes transduction of primary T cells with the produced lentiviral vectors and provides data evidencing expression of proteins encoded by each of the two genomes (e.g., transfer plasmids) packaged within the lentiviral vectors.

Four cell cultures were seeded in T225 flasks and cultured for a day. The four cell cultures were then each transfected with a transfection mixture that included a lentiviral vector system for the production of lentiviral vectors. The vCAR lentiviral vector system included a packaging plasmid, an envelope plasmid, and two transfer plasmids. See, e.g., FIG. 2. The first transfer plasmid (Transfer Plasmid 1) encoded CD47 protein and a BCMA CAR. The second transfer plasmid (Transfer Plasmid 2) encoded CD47 protein and a GPRC5D CAR. The transfection mixture for each of the four cell cultures included different ratios of Transfer Plasmid 1 and Transfer Plasmid 2, as shown below:

| Producer Cell | Transfection |
|---|---|
| Culture | Mixture Composition |
| Culture 1 | 100% Transfer Plasmid 1 |
| Culture 2 | 25% Transfer Plasmid 1/ |
| | 75% Transfer Plasmid 2 |
| Culture 3 | 50% Transfer Plasmid 1/ |
| | 50% Transfer Plasmid 2 |
| Culture 4 | 100% Transfer Plasmid 2 |

Following transfection, the four producer cell cultures were further cultured for two days to allow for the production of lentiviral vectors. Supernatant comprising the produced lentiviral vectors was then collected from each of the cultures and enriched (e.g., using chromatographic and filtration) to obtain concentrated lentiviral vector compositions (Lentiviral Composition 1, Lentiviral Composition 2, Lentiviral Composition 3, Lentiviral Composition 4).

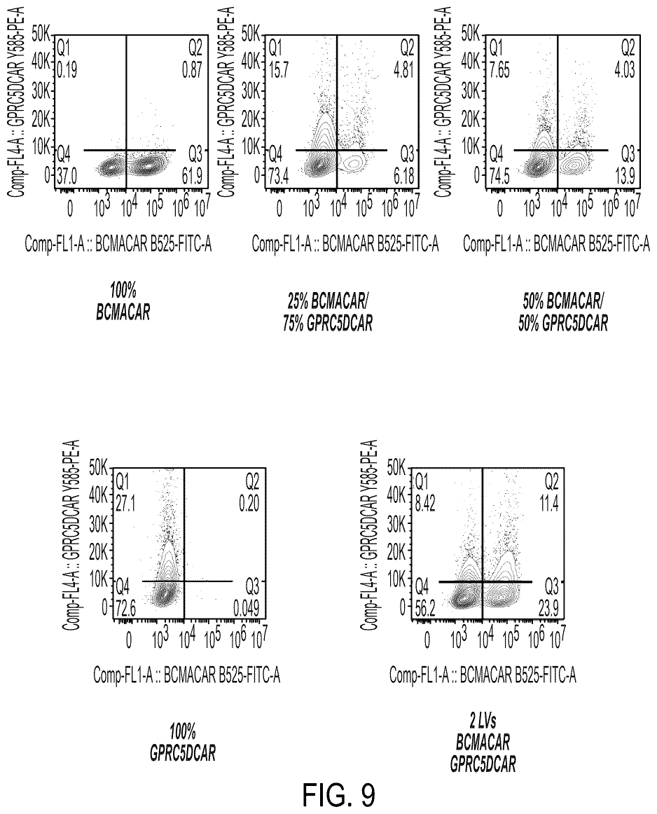

Primary T cells were then transduced with Lentiviral Composition 1, Lentiviral Composition 2, Lentiviral Composition 3, Lentiviral Composition 4, or both Lentiviral Compositions 1 and 4 in triplicate. After 72 hours, half the cells in each culture were taken out for vector copy number analysis (data not shown). Following an additional 72 hours, the remaining cells were collected and stained for flow cytometry. The cells were analyzed for BCMA CAR and GPRC5D CAR expression. As shown in FIG. 9, the transduced cells were able to doubly express both BCMA CAR and GPRC5D CAR. Further, the ratio of Transfer Plasmid 1 and Transfer Plasmid 2 within the lentiviral vectors used for transduction influenced the expression levels of encoded BCMA CAR and GPRC5D CAR from the transduced cells. For example, as the ratio of Transfer Plasmid 1 (encoding BCMA CAR) to Transfer Plasmid 2 (encoding GPRC5D CAR) in the transfection mixture (and in turn, the Lentiviral Composition) increases, the amount of BCMA CAR expressed also increases, as shown by the shift in the cell populations to the right. Similarly, as the ratio of Transfer Plasmid 1 (encoding BCMA CAR) to Transfer Plasmid 2 (encoding GPRC5D CAR) in the transfection mixture (and in turn, the Lentiviral Composition) increases, the amount of GPRC5D CAR expressed decreases, as shown by the shift in the cell populations to toward the bottom of the graphs.

The data provided herein demonstrates that proteins encoded on two genomes in a lentiviral vector can be expressed following delivery of the genomes to a cell. The data further demonstrates that expression ratios of multiple proteins encoded by two genomes in a lentiviral vector can be adjusted by adjusting the ratio of the two genomes within the lentiviral vector.

This data indicates that, by tuning the ratios of the two transfer plasmids, the expression of proteins encoding on transfer plasmids can be skewed in cells, e.g., favoring one protein over another. Importantly, the tuned co-transfection method described in this Example allows the generation of these cells expressing multiple transgenes from a single heterogeneous lentiviral vector pool following a single transduction step. This simple method has the potential to overcome biological, CMC, safety and limitations of other approaches used to generate therapeutic cells with multiple transgenes.

Sequences

| SEQ | | |
|---|---|---|
| ID | | |
| NO. | SEQUENCE | Description |
| 1 | MGPAENKKVR FENTTSDKGK IPSKVIKSYY GTMDIKKINE GLLDSKILSA | NiVG protein |
| | FNTVIALLGS IVIIVMNIMI IQNYTRSTDN QAVIKDALQG IQQQIKGLAD | attachment |
| | KIGTEIGPKV SLIDTSSTIT IPANIGLLGS KISQSTASIN ENVNEKCKFT | glycoprotein |
| | LPPLKIHECN ISCPNPLPFR EYRPQTEGVS NLVGLPNNIC LQKTSNQILK | (602 aa) |
| | PKLISYTLPV VGQSGTCITD PLLAMDEGYF AYSHLERIGS CSRGVSKQRI | |
| | IGVGEVLDRG DEVPSLFMTN VWTPPNPNTV YHCSAVYNNE FYYVLCAVST | |
| | VGDPILNSTY WSGSLMMTRL AVKPKSNGGG YNQHQLALRS IEKGRYDKVM | |
| | PYGPSGIKQG DTLYFPAVGF LVRTEFKYND SNCPITKCQY SKPENCRLSM | |
| | GIRPNSHYIL RSGLLKYNLS DGENPKVVFI EISDQRLSIG SPSKIYDSLG | |
| | QPVFYQASFS WDTMIKFGDV LTVNPLVVNW RNNTVISRPG QSQCPRFNTC | |
| | PEICWEGVYN DAFLIDRINW ISAGVFLDSN QTAENPVFTV FKDNEILYRA | |
| | QLASEDTNAQ KTITNCFLLK NKIWCISLVE IYDTGDNVIR PKLFAVKIPE QCT | |
| 2 | MMADSKLVSLNNNLSGKIKDQGKVIKNYYGTMDIKKINDGLLDSKILGAFNT | Hendra Virus G |
| | VIALLGSIIIIVMNIMIIQNYTRTTDNQALIKESLQSVQQQIKALTDKIGTEIGPKV | Protein |
| | SLIDTSSTITIPANIGLLGSKISQSTSSINENVNDKCKFTLPPLKIHECNISCPNPLP | |
| | FREYRPISQGVSDLVGLPNQICLQKTTSTILKPRLISYTLPINTREGVCITDPLLA | |
| | VDNGFFAYSHLEKIGSCTRGIAKQRIIGVGEVLDRGDKVPSMFMTNVWTPPNP | |
| | STIHHCSSTYHEDFYYTLCAVSHVGDPILNSTSWTESLSLIRLAVRPKSDSGDY | |
| | NQKYIAITKVERGKYDKVMPYGPSGIKQGDTLYFPAVGFLPRTEFQYNDSNCPI | |
| | IHCKYSKAENCRLSMGVNSKSHYILRSGLLKYNLSLGGDIILQFIEIADNRLTIG | |
| | SPSKIYNSLGQPVFYQASYSWDTMIKLGDVDTVDPLRVQWRNNSVISRPGQSQ | |
| | CPRFNVCPEVCWEGTYNDAFLIDRLNWVSAGVYLNSNQTAENPVFAVFKDNE | |
| | ILYQVPLAEDDTNAQKTITDCFLLENVIWCISLVEIYDTGDSVIRPKLFAVKIPAQ | |
| | CSES | |
| 3 | MADSKLVSLNNNLSGKIKDQGKVIKNYYGTMDIKKINDGLLDSKILGAFNTVI | Hendra Virus G |
| | ALLGSIIIIVMNIMIIQNYTRTTDNQALIKESLQSVQQQIKALTDKIGTEIGPKVSL | Protein without |
| | IDTSSTITIPANIGLLGSKISQSTSSINENVNDKCKFTLPPLKIHECNISCPNPLPFR | Met |
| | EYRPISQGVSDLVGLPNQICLQKTTSTILKPRLISYTLPINTREGVCITDPLLAVD | |
| | NGFFAYSHLEKIGSCTRGIAKQRIIGVGEVLDRGDKVPSMFMTNVWTPPNPSTI | |
| | HHCSSTYHEDFYYTLCAVSHVGDPILNSTSWTESLSLIRLAVRPKSDSGDYNQK | |
| | YIAITKVERGKYDKVMPYGPSGIKQGDTLYFPAVGFLPRTEFQYNDSNCPIIHC | |
| | KYSKAENCRLSMGVNSKSHYILRSGLLKYNLSLGGDIILQFIEIADNRLTIGSPS | |
| | KIYNSLGQPVFYQASYSWDTMIKLGDVDTVDPLRVQWRNNSVISRPGQSQCP | |
| | RFNVCPEVCWEGTYNDAFLIDRLNWVSAGVYLNSNQTAENPVFAVFKDNEIL | |
| | YQVPLAEDDTNAQKTITDCFLLENVIWCISLVEIYDTGDSVIRPKLFAVKIPAQC | |
| | SES | |
| 4 | MPAENKKVRFENTTSDKGKIPSKVIKSYYGTMDIKKINEGLLDSKILSAFNTVI | Nipah Virus G |
| | ALLGSIVIIVMNIMIIQNYTRSTDNQAVIKDALQGIQQQIKGLADKIGTEIGPKVS | Protein |
| | LIDTSSTITIPANIGLLGSKISQSTASINENVNEKCKFTLPPLKIHECNISCPNPLPF | |
| | REYRPQTEGVSNLVGLPNNICLQKTSNQILKPKLISYTLPVVGQSGTCITDPLLA | |
| | MDEGYFAYSHLERIGSCSRGVSKQRIIGVGEVLDRGDEVPSLFMTNVWTPPNP | |
| | NTVYHCSAVYNNEFYYVLCAVSTVGDPILNSTYWSGSLMMTRLAVKPKSNGG | |
| | GYNQHQLALRSIEKGRYDKVMPYGPSGIKQGDTLYFPAVGFLVRTEFKYNDSN | |
| | CPITKCQYSKPENCRLSMGIRPNSHYILRSGLLKYNLSDGENPKVVFIEISDQRL | |
| | SIGSPSKIYDSLGQPVFYQASFSWDTMIKFGDVLTVNPLVVNWRNNTVISRPG | |
| | QSQCPRFNTCPEICWEGVYNDAFLIDRINWISAGVFLDSNQTAENPVFTVFKD | |
| | NEILYRAQLASEDTNAQKTITNCFLLKNKIWCISLVEIYDTGDNVIRPKLFAVKI | |
| | PEQCT | |
| 5 | PAENKKVRFENTTSDKGKIPSKVIKSYYGTMDIKKINEGLLDSKILSAFNTVIAL | Nipah Virus G |
| | LGSIVIIVMNIMIIQNYTRSTDNQAVIKDALQGIQQQIKGLADKIGTEIGPKVSLI | Protein (No |
| | DTSSTITIPANIGLLGSKISQSTASINENVNEKCKFTLPPLKIHECNISCPNPLPFRE | Met) |
| | YRPQTEGVSNLVGLPNNICLQKTSNQILKPKLISYTLPVVGQSGTCITDPLLAM | |
| | DEGYFAYSHLERIGSCSRGVSKQRIIGVGEVLDRGDEVPSLFMTNVWTPPNPN | |
| | TVYHCSAVYNNEFYYVLCAVSTVGDPILNSTYWSGSLMMTRLAVKPKSNGGG | |
| | YNQHQLALRSIEKGRYDKVMPYGPSGIKQGDTLYFPAVGFLVRTEFKYNDSNC | |
| | PITKCQYSKPENCRLSMGIRPNSHYILRSGLLKYNLSDGENPKVVFIEISDQRLS | |
| | IGSPSKIYDSLGQPVFYQASFSWDTMIKFGDVLTVNPLVVNWRNNTVISRPGQ | |
| | SQCPRFNTCPEICWEGVYNDAFLIDRINWISAGVFLDSNQTAENPVFTVFKDNE | |
| | ILYRAQLASEDTNAQKTITNCFLLKNKIWCISLVEIYDTGDNVIRPKLFAVKIPE | |
| | QCT | |
| 6 | MLSQLQKNYLDNSNQQGDKMNNPDKKLSVNFNPLELDKGQKDLNKSYYVK | Cedar Virus G |
| | NKNYNVSNLLNESLHDIKFCIYCIFSLLIIITIINIITISIVITRLKVHEENNGMESP | Protein |
| | NLQSIQDSLSSLTNMINTEITPRIGILVTATSVTLSSSINYVGTKTNQLVNELKDYI | |
| | TKSCGFKVPELKLHECNISCADPKISKSAMYSTNAYAELAGPPKIFCKSVSKDP | |
| | DFRLKQIDYVIPVQQDRSICMNNPLLDISDGFFTYIHYEGINSCKKSDSFKVLLS | |
| | HGEIVDRGDYRPSLYLLSSHYHPYSMQVINCVPVTCNQSSFVFCHISNNTKTLD | |
| | NSDYSSDEYYITYFNGIDRPKTKKIPINNMTADNRYIHFTFSGGGGVCLGEEFII | |
| | PVTTVINTDVFTHDYCESFNCSVQTGKSLKEICSESLRSPTNSSRYNLNGIMIIS | |
| | QNNMTDFKIQLNGITYNKLSFGSPGRLSKTLGQVLYYQSSMSWDTYLKAGFV | |
| | EKWKPFTPNWMNNTVISRPNQGNCPRYHKCPEICYGGTYNDIAPLDLGKDMY | |
| | VSVILDSDQLAENPEITVFNSTTILYKERVSKDELNTRSTTTSCFLFLDEPWCISV | |
| | LETNRFNGKSIRPEIYSYKIPKYC | |
| 7 | LSQLQKNYLDNSNQQGDKMNNPDKKLSVNFNPLELDKGQKDLNKSYYVKN | Cedar Virus G |
| | KNYNVSNLLNESLHDIKFCIYCIFSLLIIITIINIITISIVITRLKVHEENNGMESPNL | Protein (No |
| | QSIQDSLSSLTNMINTEITPRIGILVTATSVTLSSSINYVGTKTNQLVNELKDYITK | Met) |
| | SCGFKVPELKLHECNISCADPKISKSAMYSTNAYAELAGPPKIFCKSVSKDPDF | |
| | RLKQIDYVIPVQQDRSICMNNPLLDISDGFFTYIHYEGINSCKKSDSFKVLLSHG | |
| | EIVDRGDYRPSLYLLSSHYHPYSMQVINCVPVTCNQSSFVFCHISNNTKTLDNS | |
| | DYSSDEYYITYFNGIDRPKTKKIPINNMTADNRYIHFTFSGGGGVCLGEEFIIPV | |
| | TTVINTDVFTHDYCESFNCSVQTGKSLKEICSESLRSPTNSSRYNLNGIMIISQN | |
| | NMTDFKIQLNGITYNKLSFGSPGRLSKTLGQVLYYQSSMSWDTYLKAGFVEK | |
| | WKPFTPNWMNNTVISRPNQGNCPRYHKCPEICYGGTYNDIAPLDLGKDMYVS | |
| | VILDSDQLAENPEITVFNSTTILYKERVSKDELNTRSTTTSCFLFLDEPWCISVLE | |
| | TNRFNGKSIRPEIYSYKIPKYC | |
| 8 | MPQKTVEFINMNSPLERGVSTLSDKKTLNQSKITKQGYFGLGSHSERNWKKQ | Bat |
| | KNQNDHYMTVSTMILEILVVLGIMENLIVLTMVYYQNDNINQRMAELTSNITV | Paramyxovirus |
| | LNLNLNQLTNKIQREIIPRITLIDTATTITIPSAITYILATLTTRISELLPSINQKCEFK | G Protein |
| | TPTLVLNDCRINCTPPLNPSDGVKMSSLATNLVAHGPSPCRNFSSVPTIYYYRIP | |
| | GLYNRTALDERCILNPRLTISSTKFAYVHSEYDKNCTRGFKYYELMTFGEILEG | |
| | PEKEPRMFSRSFYSPTNAVNYHSCTPIVTVNEGYFLCLECTSSDPLYKANLSNS | |
| | TFHLVILRHNKDEKIVSMPSFNLSTDQEYVQIIPAEGGGTAESGNLYFPCIGRLL | |
| | HKRVTHPLCKKSNCSRTDDESCLKSYYNQGSPQHQVVNCLIRIRNAQRDNPT | |
| | WDVITVDLTNTYPGSRSRIFGSFSKPMLYQSSVSWHTLLQVAEITDLDKYQLD | |
| | WLDTPYISRPGGSECPFGNYCPTVCWEGTYNDVYSLTPNNDLFVTVYLKSEQ | |
| | VAENPYFAIFSRDQILKEFPLDAWISSARTTTISCFMFNNEIWCIAALEITRLNDD | |
| | IIRPIYYSFWLPTDCRTPYPHTGKMTRVPLRSTYNY | |
| 9 | PQKTVEFINMNSPLERGVSTLSDKKTLNQSKITKQGYFGLGSHSERNWKKQK | Bat |
| | NQNDHYMTVSTMILEILVVLGIMENLIVLTMVYYQNDNINQRMAELTSNITVL | Paramyxovirus |
| | NLNLNQLTNKIQREIIPRITLIDTATTITIPSAITYILATLTTRISELLPSINQKCEFKT | G Protein (No |
| | PTLVLNDCRINCTPPLNPSDGVKMSSLATNLVAHGPSPCRNFSSVPTIYYYRIPG | Met) |
| | LYNRTALDERCILNPRLTISSTKFAYVHSEYDKNCTRGFKYYELMTFGEILEGPE | |
| | KEPRMFSRSFYSPTNAVNYHSCTPIVTVNEGYFLCLECTSSDPLYKANLSNSTF | |
| | HLVILRHNKDEKIVSMPSFNLSTDQEYVQIIPAEGGGTAESGNLYFPCIGRLLHK | |
| | RVTHPLCKKSNCSRTDDESCLKSYYNQGSPQHQVVNCLIRIRNAQRDNPTWD | |
| | VITVDLTNTYPGSRSRIFGSFSKPMLYQSSVSWHTLLQVAEITDLDKYQLDWLD | |
| | TPYISRPGGSECPFGNYCPTVCWEGTYNDVYSLTPNNDLFVTVYLKSEQVAEN | |
| | PYFAIFSRDQILKEFPLDAWISSARTTTISCFMFNNEIWCIAALEITRLNDDIIRPI | |
| | YYSFWLPTDCRTPYPHTGKMTRVPLRSTYNY | |
| 10 | MATNRDNTITSAEVSQEDKVKKYYGVETAEKVADSISGNKVFILMNTLLILTG | Mojiang virus, |
| | AIITITLNITNLTAAKSQQNMLKIIQDDVNAKLEMFVNLDQLVKGEIKPKVSLIN | Tongguan 1 G |
| | TAVSVSIPGQISNLQTKFLQKYVYLEESITKQCTCNPLSGIFPTSGPTYPPTDKPD | Protein |
| | DDTTDDDKVDTTIKPIEYPKPDGCNRTGDHFTMEPGANFYTVPNLGPASSNSD | |
| | ECYTNPSFSIGSSIYMFSQEIRKTDCTAGEILSIQIVLGRIVDKGQQGPQASPLLV | |
| | WAVPNPKIINSCAVAAGDEMGWVLCSVTLTAASGEPIPHMEDGFWLYKLEPDT | |
| | EVVSYRITGYAYLLDKQYDSVFIGKGGGIQKGNDLYFQMYGLSRNRQSFKAL | |
| | CEHGSCLGTGGGGYQVLCDRAVMSFGSEESLITNAYLKVNDLASGKPVIIGQT | |
| | FPPSDSYKGSNGRMYTIGDKYGLYLAPSSWNRYLRFGITPDISVRSTTWLKSQ | |
| | DPIMKILSTCTNTDRDMCPEICNTRGYQDIFPLSEDSEYYTYIGITPNNGGTKNF | |
| | VAVRDSDGHIASIDILQNYYSITSATISCFMYKDEIWCIAITEGKKQKDNPQRIY | |
| | AHSYKIRQMCYNMKSATVTVGNAKNITIRRY | |
| 11 | ATNRDNTITSAEVSQEDKVKKYYGVETAEKVADSISGNKVFILMNTLLILTGAII | Mojiang virus, |
| | TITLNITNLTAAKSQQNMLKIIQDDVNAKLEMFVNLDQLVKGEIKPKVSLINTA | Tongguan 1 G |
| | VSVSIPGQISNLQTKFLQKYVYLEESITKQCTCNPLSGIFPTSGPTYPPTDKPDD | (No Met) |
| | DTTDDDKVDTTIKPIEYPKPDGCNRTGDHFTMEPGANFYTVPNLGPASSNSDE | |
| | CYTNPSFSIGSSIYMFSQEIRKTDCTAGEILSIQIVLGRIVDKGQQGPQASPLLVW | |
| | AVPNPKIINSCAVAAGDEMGWVLCSVTLTAASGEPIPHMFDGFWLYKLEPDTE | |
| | VVSYRITGYAYLLDKQYDSVFIGKGGGIQKGNDLYFQMYGLSRNRQSFKALC | |
| | EHGSCLGTGGGGYQVLCDRAVMSFGSEESLITNAYLKVNDLASGKPVIIGQTF | |
| | PPSDSYKGSNGRMYTIGDKYGLYLAPSSWNRYLRFGITPDISVRSTTWLKSQD | |
| | PIMKILSTCTNTDRDMCPEICNTRGYQDIFPLSEDSEYYTYIGITPNNGGTKNFV | |
| | AVRDSDGHIASIDILQNYYSITSATISCFMYKDEIWCIAITEGKKQKDNPQRIYA | |
| | HSYKIRQMCYNMKSATVTVGNAKNITIRRY | |
| 12 | MKVR FENTTSDKGK IPSKVIKSYY GTMDIKKINE GLLDSKILSA | NiVG protein |
| | FNTVIALLGS IVIIVMNIMI IQNYTRSTDN QAVIKDALQG IQQQIKGLAD | attachment |
| | KIGTEIGPKV SLIDTSSTIT IPANIGLLGS KISQSTASIN ENVNEKCKFT | glycoprotein |
| | LPPLKIHECN ISCPNPLPFR EYRPQTEGVS NLVGLPNNIC LQKTSNQILK | Truncated Δ5 |
| | PKLISYTLPV VGQSGTCITD PLLAMDEGYF AYSHLERIGS CSRGVSKQRI | |
| | IGVGEVLDRG DEVPSLFMTN VWTPPNPNTV YHCSAVYNNE FYYVLCAVST | |
| | VGDPILNSTY WSGSLMMTRL AVKPKSNGGG YNQHQLALRS IEKGRYDKVM | |
| | PYGPSGIKQG DTLYFPAVGF LVRTEFKYND SNCPITKCQY SKPENCRLSM | |
| | GIRPNSHYIL RSGLLKYNLS DGENPKVVFI EISDQRLSIG SPSKIYDSLG | |
| | QPVFYQASFS WDTMIKFGDV LTVNPLVVNW RNNTVISRPG QSQCPRENTC | |
| | PEICWEGVYN DAFLIDRINW ISAGVFLDSN QTAENPVFTV FKDNEILYRA | |
| | QLASEDTNAQ KTITNCFLLK NKIWCISLVE IYDTGDNVIR PKLFAVKIPE QCT | |
| 13 | MSKVIKSYY GTMDIKKINE GLLDSKILSA FNTVIALLGS IVIIVMNIMI | NiVG protein |
| | IQNYTRSTDN QAVIKDALQG IQQQIKGLAD KIGTEIGPKV SLIDTSSTIT | attachment |
| | IPANIGLLGS KISQSTASIN ENVNEKCKFT LPPLKIHECN ISCPNPLPFR | glycoprotein |
| | EYRPQTEGVS NLVGLPNNIC LQKTSNQILK PKLISYTLPV VGQSGTCITD | Truncated Δ20 |
| | PLLAMDEGYF AYSHLERIGS CSRGVSKQRI IGVGEVLDRG DEVPSLFMTN | |
| | VWTPPNPNTV YHCSAVYNNE FYYVLCAVST VGDPILNSTY WSGSLMMTRL | |
| | AVKPKSNGGG YNQHQLALRS IEKGRYDKVM PYGPSGIKQG DTLYFPAVGF | |
| | LVRTEFKYND SNCPITKCQY SKPENCRLSM GIRPNSHYIL RSGLLKYNLS | |
| | DGENPKVVFI EISDQRLSIG SPSKIYDSLG QPVFYQASFS WDTMIKFGDV | |
| | LTVNPLVVNW RNNTVISRPG QSQCPRENTC PEICWEGVYN DAFLIDRINW | |
| | ISAGVFLDSN QTAENPVFTV FKDNEILYRA QLASEDTNAQ KTITNCFLLK | |
| | NKIWCISLVE IYDTGDNVIR PKLFAVKIPE QCT | |
| 14 | MSYY GTMDIKKINE GLLDSKILSA FNTVIALLGS IVIIVMNIMI | NiVG protein |
| | IQNYTRSTDN QAVIKDALQG IQQQIKGLAD KIGTEIGPKV SLIDTSSTIT | attachment |
| | IPANIGLLGS KISQSTASIN ENVNEKCKFT LPPLKIHECN ISCPNPLPFR | glycoprotein |
| | EYRPQTEGVS NLVGLPNNIC LQKTSNQILK PKLISYTLPV VGQSGTCITD | Truncated Δ25 |
| | PLLAMDEGYF AYSHLERIGS CSRGVSKQRI IGVGEVLDRG DEVPSLEMTN | |
| | VWTPPNPNTV YHCSAVYNNE FYYVLCAVST VGDPILNSTY WSGSLMMTRL | |
| | AVKPKSNGGG YNQHQLALRS IEKGRYDKVM PYGPSGIKQG DTLYFPAVGF | |
| | LVRTEFKYND SNCPITKCQY SKPENCRLSM GIRPNSHYIL RSGLLKYNLS | |
| | DGENPKVVFI EISDQRLSIG SPSKIYDSLG QPVFYQASFS WDTMIKFGDV | |
| | LTVNPLVVNW RNNTVISRPG QSQCPRENTC PEICWEGVYN DAFLIDRINW | |
| | ISAGVFLDSN QTAENPVFTV FKDNEILYRA QLASEDTNAQ KTITNCFLLK | |
| | NKIWCISLVE IYDTGDNVIR PKLFAVKIPE QCT | |
| 15 | ILHY EKLSKIGLVK GVTRKYKIKS NPLTKDIVIK MIPNVSNMSQ | Nipah virus NiV- |
| | CTGSVMENYK TRLNGILTPI KGALEIYKNQ THDLVGDVRL AGVIMAGVAI | F F0 T234 |
| | GIATAAQITA GVALYEAMKN ADNINKLKSS IESTNEAVVK LQETAEKTVY | truncation (aa |
| | VLTALQDYIN TNLVPTIDKI SCKQTELSLD LALSKYLSDL LFVFGPNLQD | 525-544) AND |
| | PVSNSMTIQA ISQAFGGNYE TLLRTLGYAT EDFDDLLESD SITGQIIYVD | mutation on N- |
| | LSSYYIIVRV YFPILTEIQQ AYIQELLPVS FNNDNSEWIS IVPNFILVRN | linked |
| | TLISNIEIGF CLITKRSVIC NQDYATPMTN NMRECLTGST EKCPRELVVS | glycosylation |
| | SHVPRFALSN GVLFANCISV TCQCQTTGRA ISQSGEQTLL MIDNTTCPTA | site |
| | VLGNVIISLG KYLGSVNYNS EGIAIGPPVF TDKVDISSQI SSMNQSLQQS | |
| | KDYIKEAQRL LDTVNPSLIS MLSMIILYVL SIASLCIGLI TFISFIIVEK KRNTGT | |
| 16 | MVVILDKRCY CNLLILILMI SECSVGILHY EKLSKIGLVK GVTRKYKIKS | Truncated NiV |
| | NPLTKDIVIK MIPNVSNMSQ CTGSVMENYK TRLNGILTPI KGALEIYKNN | fusion |
| | THDLVGDVRL AGVIMAGVAI GIATAAQITA GVALYEAMKN ADNINKLKSS | glycoprotein |
| | IESTNEAVVK LQETAEKTVY VLTALQDYIN TNLVPTIDKI SCKQTELSLD | (FcDelta22) at |
| | LALSKYLSDL LFVFGPNLQD PVSNSMTIQA ISQAFGGNYE TLLRTLGYAT | cytoplasmic tail |
| | EDFDDLLESD SITGQIIYVD LSSYYIIVRV YFPILTEIQQ AYIQELLPVS | (with signal |
| | FNNDNSEWIS IVPNFILVRN TLISNIEIGF CLITKRSVIC NQDYATPMTN | sequence) |
| | NMRECLTGST EKCPRELVVS SHVPRFALSN GVLFANCISV TCQCQTTGRA | |
| | ISQSGEQTLL MIDNTTCPTA VLGNVIISLG KYLGSVNYNS EGIAIGPPVF | |
| | TDKVDISSQI SSMNQSLQQS KDYIKEAQRL LDTVNPSLIS MLSMIILYVL | |
| | SIASLCIGLI TFISFIIVEK KRNT | |
| 17 | MKKINEGLLDSKILSA FNTVIALLGS IVIIVMNIMI IQNYTRSTDN | NiVG protein |
| | QAVIKDALQG IQQQIKGLAD KIGTEIGPKV SLIDTSSTIT IPANIGLLGS | attachment |
| | KISQSTASIN ENVNEKCKFT LPPLKIHECN ISCPNPLPFR EYRPQTEGVS | glycoprotein |
| | NLVGLPNNIC LQKTSNQILK PKLISYTLPV VGQSGTCITD PLLAMDEGYF | Truncated and |
| | AYSHLERIGS CSRGVSKQRI IGVGEVLDRG DEVPSLFMTN VWTPPNPNTV | mutated |
| | YHCSAVYNNE FYYVLCAVST VGDPILNSTY WSGSLMMTRL AVKPKSNGGG | (E501 A, |
| | YNQHQLALRS IEKGRYDKVM PYGPSGIKQG DTLYFPAVGF LVRTEFKYND | W504A, Q530A, |
| | SNCPITKCQY SKPENCRLSM GIRPNSHYIL RSGLLKYNLS DGENPKVVFI | E533A) NIV G |
| | EISDQRLSIG SPSKIYDSLG QPVFYQASFS WDTMIKIGDV LTVNPLVVNW | protein (Gc Δ |
| | RNNTVISRPG QSQCPRENTC PAICAEGVYN DAFLIDRINW ISAGVFLDSN | 34) |
| | ATAANPVFTV FKDNEILYRA QLASEDTNAQ KTITNCFLLK NKIWCISLVE | |
| | IYDTGDNVIR PKLFAVKIPE QCT | |
| 18 | KKINEGLLDSKILSA FNTVIALLGS IVIIVMNIMI IQNYTRSTDN | NiVG protein |
| | QAVIKDALQG IQQQIKGLAD KIGTEIGPKV SLIDTSSTIT IPANIGLLGS | attachment |
| | KISQSTASIN ENVNEKCKFT LPPLKIHECN ISCPNPLPFR EYRPQTEGVS | glycoprotein |
| | NLVGLPNNIC LQKTSNQILK PKLISYTLPV VGQSGTCITD PLLAMDEGYF | Truncated and |
| | AYSHLERIGS CSRGVSKQRI IGVGEVLDRG DEVPSLFMTN VWTPPNPNTV | mutated |
| | YHCSAVYNNE FYYVLCAVST VGDPILNSTY WSGSLMMTRL AVKPKSNGGG | (E501 A, |
| | YNQHQLALRS IEKGRYDKVM PYGPSGIKQG DTLYFPAVGF LVRTEFKYND | W504A, Q530A, |
| | SNCPITKCQY SKPENCRLSM GIRPNSHYIL RSGLLKYNLS DGENPKVVFI | E533A) NiV G |
| | EISDQRLSIG SPSKIYDSLG QPVFYQASFS WDTMIKFGDV LTVNPLVVNW | protein (Gc Δ |
| | RNNTVISRPG QSQCPRENTC PAICAEGVYN DAFLIDRINW ISAGVFLDSN | 34) Without N- |
| | ATAANPVFTV FKDNEILYRA QLASEDTNAQ KTITNCFLLK NKIWCISLVE | terminal |
| | IYDTGDNVIR PKLFAVKIPE QCT | methionine |
| 19 | MVVILDKRCY CNLLILILMI SECSVGILHY EKLSKIGLVK GVTRKYKIKS | Truncated NiV |
| | NPLTKDIVIK MIPNVSNMSQ CTGSVMENYK TRLNGILTPI KGALEIYKNN | fusion |
| | THDLVGDVRL AGVIMAGVAI GIATAAQITA GVALYEAMKN ADNINKLKSS | glycoprotein |
| | IESTNEAVVK LQETAEKTVY VLTALQDYIN TNLVPTIDKI SCKQTELSLD | (FcDelta22) at |
| | LALSKYLSDL LFVFGPNLQD PVSNSMTIQA ISQAFGGNYE TLLRTLGYAT | cytoplasmic tail |
| | EDFDDLLESD SITGQIIYVD LSSYYIIVRV YFPILTEIQQ AYIQELLPVS | (with signal |
| | FNNDNSEWIS IVPNFILVRN TLISNIEIGF CLITKRSVIC NQDYATPMTN | sequence) |
| | NMRECLTGST EKCPRELVVS SHVPRFALSN GVLFANCISV TCQCQTTGRA | |
| | ISQSGEQTLL MIDNTTCPTA VLGNVIISLG KYLGSVNYNS EGIAIGPPVF | |
| | TDKVDISSQI SSMNQSLQQS KDYIKEAQRL LDTVNPSLIS MLSMIILYVL | |
| | SIASLCIGLI TFISFIIVEK KRNT | |
| 20 | ILHY EKLSKIGLVK GVTRKYKIKS NPLTKDIVIK MIPNVSNMSQ | Nipah virus NiV- |
| | CTGSVMENYK TRLNGILTPI KGALEIYKNN THDLVGDVRL AGVIMAGVAI | F F0 T234 |
| | GIATAAQITA GVALYEAMKN ADNINKLKSS IESTNEAVVK LQETAEKTVY | truncation (aa |
| | VLTALQDYIN TNLVPTIDKI SCKQTELSLD LALSKYLSDL LFVFGPNLQD | 525-544) |
| | PVSNSMTIQA ISQAFGGNYE TLLRTLGYAT EDFDDLLESD SITGQIIYVD | |
| | LSSYYIIVRV YFPILTEIQQ AYIQELLPVS FNNDNSEWIS IVPNFILVRN | |
| | TLISNIEIGF CLITKRSVIC NQDYATPMTN NMRECLTGST EKCPRELVVS | |
| | SHVPRFALSN GVLFANCISV TCQCQTTGRA ISQSGEQTLL MIDNTTCPTA | |
| | VLGNVIISLG KYLGSVNYNS EGIAIGPPVF TDKVDISSQI SSMNQSLQQS | |
| | KDYIKEAQRL LDTVNPSLIS MLSMIILYVL SIASLCIGLI TFISFIIVEK KRNTGT | |
| 21 | ILHY EKLSKIGLVK GVTRKYKIKS NPLTKDIVIK MIPNVSNMSQ | Truncated |
| | CTGSVMENYK TRLNGILTPI KGALEIYKNN THDLVGDVRL AGVIMAGVAI | mature NiV |
| | GIATAAQITA GVALYEAMKN ADNINKLKSS IESTNEAVVK LQETAEKTVY | fusion |
| | VLTALQDYIN TNLVPTIDKI SCKQTELSLD LALSKYLSDL LFVFGPNLQD | glycoprotein |
| | PVSNSMTIQA ISQAFGGNYE TLLRTLGYAT EDFDDLLESD SITGQIIYVD | (FcDelta22) at |
| | LSSYYIIVRV YFPILTEIQQ AYIQELLPVS FNNDNSEWIS IVPNFILVRN | cytoplasmic tail |
| | TLISNIEIGF CLITKRSVIC NQDYATPMTN NMRECLTGST EKCPRELVVS | |
| | SHVPRFALSN GVLFANCISV TCQCQTTGRA ISQSGEQTLL MIDNTTCPTA | |
| | VLGNVIISLG KYLGSVNYNS EGIAIGPPVF TDKVDISSQI SSMNQSLQQS | |
| | KDYIKEAQRL LDTVNPSLIS MLSMIILYVL SIASLCIGLI TFISFIIVEK KRNT | |
| 22 | FNTVIALLGS IVIIVMNIMI IQNYTRSTDN QAVIKDALQG IQQQIKGLAD | NivG protein |
| | KIGTEIGPKV SLIDTSSTIT IPANIGLLGS KISQSTASIN ENVNEKCKFT | attachment |
| | LPPLKIHECN ISCPNPLPFR EYRPQTEGVS NLVGLPNNIC LQKTSNQILK | glycoprotein |
| | PKLISYTLPV VGQSGTCITD PLLAMDEGYF AYSHLERIGS CSRGVSKQRI | Without |
| | IGVGEVLDRG DEVPSLFMTN VWTPPNPNTV YHCSAVYNNE FYYVLCAVST | cytoplasmic tail |
| | VGDPILNSTY WSGSLMMTRL AVKPKSNGGG YNQHQLALRS IEKGRYDKVM | Uniprot Q9IH62 |
| | PYGPSGIKQG DTLYFPAVGF LVRTEFKYND SNCPITKCQY SKPENCRLSM | |
| | GIRPNSHYIL RSGLLKYNLS DGENPKVVFI EISDQRLSIG SPSKIYDSLG | |
| | QPVFYQASFS WDTMIKFGDV LTVNPLVVNW RNNTVISRPG QSQCPRENTC | |
| | PEICWEGVYN DAFLIDRINW ISAGVFLDSN QTAENPVFTV FKDNEILYRA | |
| | QLASEDTNAQ KTITNCFLLK NKIWCISLVE IYDTGDNVIR PKLFAVKIPE QC | |
| 23 | MMADSKLVSL NNNLSGKIKD QGKVIKNYYG TMDIKKINDG LLDSKILGAF | Hendra virus G |
| | NTVIALLGSI IIIVMNIMII QNYTRTTDNQ ALIKESLQSV QQQIKALTDK | protein Uniprot |
| | IGTEIGPKVS LIDTSSTITI PANIGLLGSK ISQSTSSINE NVNDKCKFTL | O89343 |
| | PPLKIHECNI SCPNPLPFRE YRPISQGVSD LVGLPNQICL QKTTSTILKP | |
| | RLISYTLPIN TREGVCITDP LLAVDNGFFA YSHLEKIGSC TRGIAKQRII | |
| | GVGEVLDRGD KVPSMFMTNV WTPPNPSTIH HCSSTYHEDF YYTLCAVSHV | |
| | GDPILNSTSW TESLSLIRLA VRPKSDSGDY NQKYIAITKV ERGKYDKVMP | |
| | YGPSGIKQGD TLYFPAVGFL PRTEFQYNDS NCPIIHCKYS KAENCRLSMG | |
| | VNSKSHYILR SGLLKYNLSL GGDIILQFIE IADNRLTIGS PSKIYNSLGQ | |
| | PVFYQASYSW DTMIKLGDVD TVDPLRVQWR NNSVISRPGQ SQCPRFNVCP | |
| | EVCWEGTYND AFLIDRLNWV SAGVYLNSNQ TAENPVFAVF KDNEILYQVP | |
| | LAEDDTNAQK TITDCFLLEN VIWCISLVEI YDTGDSVIRP KLFAVKIPAQ CSES | |
| 24 | MADSKLVSL NNNLSGKIKD QGKVIKNYYG TMDIKKINDG LLDSKILGAF | Hendra virus G |
| | NTVIALLGSI IIIVMNIMII QNYTRTTDNQ ALIKESLQSV QQQIKALTDK | protein Uniprot |
| | IGTEIGPKVS LIDTSSTITI PANIGLLGSK ISQSTSSINE NVNDKCKFTL | O89343 Without |
| | PPLKIHECNI SCPNPLPFRE YRPISQGVSD LVGLPNQICL QKTTSTILKP | N-terminal |
| | RLISYTLPIN TREGVCITDP LLAVDNGFFA YSHLEKIGSC TRGIAKQRII | methionine |
| | GVGEVLDRGD KVPSMFMTNV WTPPNPSTIH HCSSTYHEDF YYTLCAVSHV | |
| | GDPILNSTSW TESLSLIRLA VRPKSDSGDY NQKYIAITKV ERGKYDKVMP | |
| | YGPSGIKQGD TLYFPAVGFL PRTEFQYNDS NCPIIHCKYS KAENCRLSMG | |
| | VNSKSHYILR SGLLKYNLSL GGDIILQFIE IADNRLTIGS PSKIYNSLGQ | |
| | PVFYQASYSW DTMIKLGDVD TVDPLRVQWR NNSVISRPGQ SQCPRFNVCP | |
| | EVCWEGTYND AFLIDRLNWV SAGVYLNSNQ TAENPVFAVF KDNEILYQVP | |
| | LAEDDTNAQK TITDCFLLEN VIWCISLVEI YDTGDSVIRP KLFAVKIPAQ CSES | |
| 25 | FNTVIALLGSI IIIVMNIMII QNYTRTTDNQ ALIKESLQSV QQQIKALTDK | Hendra virus G |
| | IGTEIGPKVS LIDTSSTITI PANIGLLGSK ISQSTSSINE NVNDKCKFTL | protein Uniprot |
| | PPLKIHECNI SCPNPLPFRE YRPISQGVSD LVGLPNQICL QKTTSTILKP | O89343 |
| | RLISYTLPIN TREGVCITDP LLAVDNGFFA YSHLEKIGSC TRGIAKQRII | Without |
| | GVGEVLDRGD KVPSMFMTNV WTPPNPSTIH HCSSTYHEDF YYTLCAVSHV | cytoplasmic tail |
| | GDPILNSTSW TESLSLIRLA VRPKSDSGDY NQKYIAITKV ERGKYDKVMP | |
| | YGPSGIKQGD TLYFPAVGFL PRTEFQYNDS NCPIIHCKYS KAENCRLSMG | |
| | VNSKSHYILR SGLLKYNLSL GGDIILQFIE IADNRLTIGS PSKIYNSLGQ | |
| | PVFYQASYSW DTMIKLGDVD TVDPLRVQWR NNSVISRPGQ SQCPRFNVCP | |
| | EVCWEGTYND AFLIDRLNWV SAGVYLNSNQ TAENPVFAVF KDNEILYQVP | |
| | LAEDDTNAQK TITDCFLLEN VIWCISLVEI YDTGDSVIRP KLFAVKIPAQ CSES | |
| 26 | FNTVIALLGSI IIIVMNIMII QNYTRTTDNQ ALIKESLQSV QQQIKALTDK | Hendra virus G |
| | IGTEIGPKVS LIDTSSTITI PANIGLLGSK ISQSTSSINE NVNDKCKFTL | protein Uniprot |
| | PPLKIHECNI SCPNPLPFRE YRPISQGVSD LVGLPNQICL QKTTSTILKP | O89343 |
| | RLISYTLPIN TREGVCITDP LLAVDNGFFA YSHLEKIGSC TRGIAKQRII | Without |
| | GVGEVLDRGD KVPSMFMTNV WTPPNPSTIH HCSSTYHEDF YYTLCAVSHV | cytoplasmic tail |
| | GDPILNSTSW TESLSLIRLA VRPKSDSGDY NQKYIAITKV ERGKYDKVMP | |
| | YGPSGIKQGD TLYFPAVGFL PRTEFQYNDS NCPIIHCKYS KAENCRLSMG | |
| | VNSKSHYILR SGLLKYNLSL GGDIILQFIE IADNRLTIGS PSKIYNSLGQ | |
| | PVFYQASYSW DTMIKLGDVD TVDPLRVQWR NNSVISRPGQ SQCPRFNVCP | |
| | EVCWEGTYND AFLIDRLNWV SAGVYLNSNQ TAENPVFAVF KDNEILYQVP | |
| | LAEDDTNAQK TITDCFLLEN VIWCISLVEI YDTGDSVIRP KLFAVKIPAQ CSES | |
| 27 | MGPAENKKVR FENTTSDKGK IPSKVIKSYY GTMDIKKINE GLLDSKILSA | NiVG protein |
| | FNTVIALLGS IVIIVMNIMI IQNYTRSTDN QAVIKDALQG IQQQIKGLAD | attachment |
| | KIGTEIGPKV SLIDTSSTIT IPANIGLLGS KISQSTASIN ENVNEKCKFT | glycoprotein |
| | LPPLKIHECN ISCPNPLPFR EYRPQTEGVS NLVGLPNNIC LQKTSNQILK | (602 aa) |
| | PKLISYTLPV VGQSGTCITD PLLAMDEGYF AYSHLERIGS CSRGVSKQRI | |
| | IGVGEVLDRG DEVPSLFMTN VWTPPNPNTV YHCSAVYNNE FYYVLCAVST | |
| | VGDPILNSTY WSGSLMMTRL AVKPKSNGGG YNQHQLALRS IEKGRYDKVM | |
| | PYGPSGIKQG DTLYFPAVGF LVRTEFKYND SNCPITKCQY SKPENCRLSM | |
| | GIRPNSHYIL RSGLLKYNLS DGENPKVVFI EISDQRLSIG SPSKIYDSLG | |
| | QPVFYQASFS WDTMIKFGDV LTVNPLVVNW RNNTVISRPG QSQCPRENTC | |
| | PEICWEGVYN DAFLIDRINW ISAGVFLDSN QTAENPVFTV FKDNEILYRA | |
| | QLASEDTNAQ KTITNCFLLK NKIWCISLVE IYDTGDNVIR PKLFAVKIPE QC | |
| 28 | MATQEVRLKCLLCGIIVLVLSLEGLGILHYEKLSKIGLVKGITRKYKIKSNPLTKDIVIKMIPNVSNVSK | Hendra virus F |
| | CTGTVMENYKSRLTGILSPIKGAIELYNNNTHDLVGDVKLAGVVMAGIAIGIATAAQITAGVALYE | Protein |
| | AMKNADNINKLKSSIESTNEAVVKLQETAEKTVYVLTALQDYINTNLVPTIDQISCKQTELALDLAL | |
| | SKYLSDLLFVFGPNLQDPVSNSMTIQAISQAFGGNYETLLRTLGYATEDFDDLLESDSIAGQIVYV | |
| | DLSSYYIIVRVYFPILTEIQQAYVQELLPVSFNNDNSEWISIVPNFVLIRNTLISNIEVKYCLITKKSVIC | |
| | NQDYATPMTASVRECLTGSTDKCPRELVVSSHVPRFALSGGVLFANCISVTCQCQTTGRAISQSG | |
| | EQTLLMIDNTTCTTVVLGNIIISLGKYLGSINYNSESIAVGPPVYTDKVDISSQISSMNQSLQQSKD | |
| | YIKEAQKILDTVNPSLISMLSMIILYVLSIAALCIGLITFISFVIVEKKRGNYSRLDDRQVRPVSNGDLY | |
| | YIGT | |
| 29 | ILHYEKLSKIGLVKGITRKYKIKSNPLTKDIVIKMIPNVSNVSKCTGTVMENYKS | Hendra virus F |
| | RLTGILSPIKGAIELYNNNTHDLVGDVKLAGVVMAGIAIGIATAAQITAGVALYE | Protein, |
| | AMKNADNINKLKSSIESTNEAVVKLQETAEKTVYVLTALQDYINTNLVPTIDQI | Without signal |
| | SCKQTELALDLALSKYLSDLLFVFGPNLQDPVSNSMTIQAISQAFGGNYETLL | sequence |
| | RTLGYATEDFDDLLESDSIAGQIVYVDLSSYYIIVRVYFPILTEIQQAYVQELLPV | |
| | SFNNDNSEWISIVPNFVLIRNTLISNIEVKYCLITKKSVICNQDYATPMTASVREC | |
| | LTGSTDKCPRELVVSSHVPRFALSGGVLFANCISVTCQCQTTGRAISQSGEQTL | |
| | LMIDNTTCTTVVLGNIIISLGKYLGSINYNSESIAVGPPVYTDKVDISSQISSMNQ | |
| | SLQQSKDYIKEAQKILDTVNPSLISMLSMIILYVLSIAALCIGLITFISFVIVEKKR | |
| | GNYSRLDDRQVRPVSNGDLYYIGT | |
| 30 | MVVILDKRCYCNLLILILMISECSVGILHYEKLSKIGLVKGVTRKYKIKSNPLTK | Nipah virus F |
| | DIVIKMIPNVSNMSQCTGSVMENYKTRLNGILTPIKGALEIYKNNTHDLVGDV | Protein |
| | RLAGVIMAGVAIGIATAAQITAGVALYEAMKNADNINKLKSSIESTNEAVVKLQ | |
| | ETAEKTVYVLTALQDYINTNLVPTIDKISCKQTELSLDLALSKYLSDLLFVFGPN | |
| | LQDPVSNSMTIQAISQAFGGNYETLLRTLGYATEDFDDLLESDSITGQIIYVDLS | |
| | SYYIIVRVYFPILTEIQQAYIQELLPVSFNNDNSEWISIVPNFILVRNTLISNIEIGF | |
| | CLITKRSVICNQDYATPMTNNMRECLTGSTEKCPRELVVSSHVPRFALSNGVLF | |
| | ANCISVTCQCQTTGRAISQSGEQTLLMIDNTTCPTAVLGNVIISLGKYLGSVNY | |
| | NSEGIAIGPPVFTDKVDISSQISSMNQSLQQSKDYIKEAQRLLDTVNPSLISMLS | |
| | MIILYVLSIASLCIGLITFISFIIVEKKRNTYSRLEDRRVRPTSSGDLYYIGT | |
| 31 | ILHYEKLSKIGLVKGVTRKYKIKSNPLTKDIVIKMIPNVSNMSQCTGSVMENYK | Nipah virus F |
| | TRLNGILTPIKGALEIYKNNTHDLVGDVRLAGVIMAGVAIGIATAAQITAGVALY | Protein, without |
| | EAMKNADNINKLKSSIESTNEAVVKLQETAEKTVYVLTALQDYINTNLVPTIDK | signal sequence |
| | ISCKQTELSLDLALSKYLSDLLFVFGPNLQDPVSNSMTIQAISQAFGGNYETLL | |
| | RTLGYATEDFDDLLESDSITGQIIYVDLSSYYIIVRVYFPILTEIQQAYIQELLPVS | |
| | FNNDNSEWISIVPNFILVRNTLISNIEIGFCLITKRSVICNQDYATPMTNNMRECL | |
| | TGSTEKCPRELVVSSHVPRFALSNGVLFANCISVTCQCQTTGRAISQSGEQTLL | |
| | MIDNTTCPTAVLGNVIISLGKYLGSVNYNSEGIAIGPPVFTDKVDISSQISSMNQ | |
| | SLQQSKDYIKEAQRLLDTVNPSLISMLSMIILYVLSIASLCIGLITFISFIIVEKKR | |
| | NTYSRLEDRRVRPTSSGDLYYIGT | |
| 32 | MSNKRTTVLIIISYTLFYLNNAAIVGFDFDKLNKIGVVQGRVLNYKIKGDPMTK | Cedar Virus F |
| | DLVLKFIPNIVNITECVREPLSRYNETVRRLLLPIHNMLGLYLNNTNAKMTGLM | Protein |
| | IAGVIMGGIAIGIATAAQITAGFALYEAKKNTENIQKLTDSIMKTQDSIDKLTDSV | |
| | GTSILILNKLQTYINNQLVPNLELLSCRQNKIEFDLMLTKYLVDLMTVIGPNINN | |
| | PVNKDMTIQSLSLLFDGNYDIMMSELGYTPQDFLDLIESKSITGQIIYVDMENL | |
| | YVVIRTYLPTLIEVPDAQIYEFNKITMSSNGGEYLSTIPNFILIRGNYMSNIDVAT | |
| | CYMTKASVICNQDYSLPMSQNLRSCYQGETEYCPVEAVIASHSPRFALTNGVIF | |
| | ANCINTICRCQDNGKTITQNINQFVSMIDNSTCNDVMVDKFTIKVGKYMGRK | |
| | DINNINIQIGPQIIIDKVDLSNEINKMNQSLKDSIFYLREAKRILDSVNISLISPSV | |
| | QLFLIIISVLSFIILLIIIVYLYCKSKHSYKYNKFIDDPDYYNDYKRERINGKASKS | |
| | NNIYYVGD | |
| 33 | TVLIIISYTLFYLNNAAIVGFDFDKLNKIGVVQGRVLNYKIKGDPMTKDLVLKF | Cedar Virus F |
| | IPNIVNITECVREPLSRYNETVRRLLLPIHNMLGLYLNNTNAKMTGLMIAGVIM | Protein, without |
| | GGIAIGIATAAQITAGFALYEAKKNTENIQKLTDSIMKTQDSIDKLTDSVGTSILI | signal sequence |
| | LNKLQTYINNQLVPNLELLSCRQNKIEFDLMLTKYLVDLMTVIGPNINNPVNK | |
| | DMTIQSLSLLFDGNYDIMMSELGYTPQDFLDLIESKSITGQIIYVDMENLYVVIR | |
| | TYLPTLIEVPDAQIYEFNKITMSSNGGEYLSTIPNFILIRGNYMSNIDVATCYMT | |
| | KASVICNQDYSLPMSQNLRSCYQGETEYCPVEAVIASHSPRFALTNGVIFANCI | |
| | NTICRCQDNGKTITQNINQFVSMIDNSTCNDVMVDKFTIKVGKYMGRKDINNI | |
| | NIQIGPQIIIDKVDLSNEINKMNQSLKDSIFYLREAKRILDSVNISLISPSVQLFLII | |
| | ISVLSFIILLIIIVYLYCKSKHSYKYNKFIDDPDYYNDYKRERINGKASKSNNIYY | |
| | VGD | |
| 34 | MALNKNMFSSLFLGYLLVYATTVQSSIHYDSLSKVGVIKGLTYNYKIKGSPSTK | Mojiang virus, |
| | LMVVKLIPNIDSVKNCTQKQYDEYKNLVRKALEPVKMAIDTMLNNVKSGNN | Tongguan 1 F |
| | KYRFAGAIMAGVALGVATAATVTAGIALHRSNENAQAIANMKSAIQNTNEAVK | Protein |
| | QLQLANKQTLAVIDTIRGEINNNIIPVINQLSCDTIGLSVGIRLTQYYSEIITAFGP | |
| | ALQNPVNTRITIQAISSVFNGNFDELLKIMGYTSGDLYEILHSELIRGNIIDVDV | |
| | DAGYIALEIEFPNLTLVPNAVVQELMPISYNIDGDEWVTLVPRFVLTRTTLLSNI | |
| | DTSRCTITDSSVICDNDYALPMSHELIGCLQGDTSKCAREKVVSSYVPKFALSD | |
| | GLVYANCLNTICRCMDTDTPISQSLGATVSLLDNKRCSVYQVGDVLISVGSYL | |
| | GDGEYNADNVELGPPIVIDKIDIGNQLAGINQTLQEAEDYIEKSEEFLKGVNPSI | |
| | ITLGSMVVLYIFMILIAIVSVIALVLSIKLTVKGNVVRQQFTYTQHVPSMENINY | |
| | VSH | |
| 35 | IHYDSLSKVGVIKGLTYNYKIKGSPSTKLMVVKLIPNIDSVKNCTQKQYDEYK | Mojiang virus, |
| | NLVRKALEPVKMAIDTMLNNVKSGNNKYRFAGAIMAGVALGVATAATVTAGI | Tongguan 1 F |
| | ALHRSNENAQAIANMKSAIQNTNEAVKQLQLANKQTLAVIDTIRGEINNNIIPV | Protein, without |
| | INQLSCDTIGLSVGIRLTQYYSEIITAFGPALQNPVNTRITIQAISSVENGNFDELL | signal sequence |
| | KIMGYTSGDLYEILHSELIRGNIIDVDVDAGYIALEIEFPNLTLVPNAVVQELMPI | |
| | SYNIDGDEWVTLVPRFVLTRTTLLSNIDTSRCTITDSSVICDNDYALPMSHELIG | |
| | CLQGDTSKCAREKVVSSYVPKFALSDGLVYANCLNTICRCMDTDTPISQSLGA | |
| | TVSLLDNKRCSVYQVGDVLISVGSYLGDGEYNADNVELGPPIVIDKIDIGNQL | |
| | AGINQTLQEAEDYIEKSEEFLKGVNPSIITLGSMVVLYIFMILIAIVSVIALVLSIK | |
| | LTVKGNVVRQQFTYTQHVPSMENINYVSH | |
| 36 | MKKKTDNPTISKRGHNHSRGIKSRALLRETDNYSNGLIVENLVRNCHHPSKNN | Bat |
| | LNYTKTQKRDSTIPYRVEERKGHYPKIKHLIDKSYKHIKRGKRRNGHNGNIITII | Paramyxovirus F |
| | LLLILILKTQMSEGAIHYETLSKIGLIKGITREYKVKGTPSSKDIVIKLIPNVTGL | Protein |
| | NKCTNISMENYKEQLDKILIPINNIIELYANSTKSAPGNARFAGVIIAGVALGVA | |
| | AAAQITAGIALHEARQNAERINLLKDSISATNNAVAELQEATGGIVNVITGMQD | |
| | YINTNLVPQIDKLQCSQIKTALDISLSQYYSEILTVFGPNLQNPVTTSMSIQAISQ | |
| | SFGGNIDLLLNLLGYTANDLLDLLESKSITGQITYINLEHYFMVIRVYYPIMTTI | |
| | SNAYVQELIKISFNVDGSEWVSLVPSYILIRNSYLSNIDISECLITKNSVICRHDF | |
| | AMPMSYTLKECLTGDTEKCPREAVVTSYVPRFAISGGVIYANCLSTTCQCYQT | |
| | GKVIAQDGSQTLMMIDNQTCSIVRIEEILISTGKYLGSQEYNTMHVSVGNPVFT | |
| | DKLDITSQISNINQSIEQSKFYLDKSKAILDKINLNLIGSVPISILFIIAILSLILSIITF | |
| | VIVMIIVRRYNKYTPLINSDPSSRRSTIQDVYIIPNPGEHSIRSAARSIDRDRD | |
| 37 | SRALLRETDNYSNGLIVENLVRNCHHPSKNNLNYTKTQKRDSTIPYRVEERKG | Bat |
| | HYPKIKHLIDKSYKHIKRGKRRNGHNGNIITIILLLILILKTQMSEGAIHYETLSK | Paramyxovirus F |
| | IGLIKGITREYKVKGTPSSKDIVIKLIPNVTGLNKCTNISMENYKEQLDKILIPIN | Protein, without |
| | NIIELYANSTKSAPGNARFAGVIIAGVALGVAAAAQITAGIALHEARQNAERINL | signal sequence |
| | LKDSISATNNAVAELQEATGGIVNVITGMQDYINTNLVPQIDKLQCSQIKTALDI | |
| | SLSQYYSEILTVFGPNLQNPVTTSMSIQAISQSFGGNIDLLLNLLGYTANDLLDL | |
| | LESKSITGQITYINLEHYFMVIRVYYPIMTTISNAYVQELIKISFNVDGSEWVSLV | |
| | PSYILIRNSYLSNIDISECLITKNSVICRHDFAMPMSYTLKECLTGDTEKCPREAV | |
| | VTSYVPRFAISGGVIYANCLSTTCQCYQTGKVIAQDGSQTLMMIDNQTCSIVRI | |
| | EEILISTGKYLGSQEYNTMHVSVGNPVFTDKLDITSQISNINQSIEQSKFYLDKS | |
| | KAILDKINLNLIGSVPISILFIIAILSLILSIITFVIVMIIVRRYNKYTPLINSDPSSRR | |
| | STIQDVYIIPNPGEHSIRSAARSIDRDRD | |
| 38 | MVVILDKRCY CNLLILILMI SECSVG | signal sequence |
| 39 | ILHYEKLSKIGLVKGVTRKYKIKSNPLTKDIVIKMIPNVSNMSQCTGSVMENYK | Nipah virus NiV- |
| | TRLNGILTPIKGALEIYKNNTHDLVGDVR | F F2 (aa 27-109) |
| 40 | MVVILDKRCYCNLLILILMISECSVGILHYEKLSKIGLVKGVTRKYKIKSNPLTK | Nipah virus F |
| | DIVIKMIPNVSNMSQCTGSVMENYKTRLNGILTPIKGALEIYKNNTHDLVGDV | Protein |
| | RLAGVIMAGVAIGIATAAQITAGVALYEAMKNADNINKLKSSIESTNEAVVKLQ | |
| | ETAEKTVYVLTALQDYINTNLVPTIDKISCKQTELSLDLALSKYLSDLLFVFGPN | |
| | LQDPVSNSMTIQAISQAFGGNYETLLRTLGYATEDFDDLLESDSITGQIIYVDLS | |
| | SYYIIVRVYFPILTEIQQAYIQELLPVSFNNDNSEWISIVPNFILVRNTLISNIEIGF | |
| | CLITKRSVICNQDYATPMTNNMRECLTGSTEKCPRELVVSSHVPRFALSNGVLF | |
| | ANCISVTCQCQTTGRAISQSGEQTLLMIDNTTCPTAVLGNVIISLGKYLGSVNY | |
| | NSEGIAIGPPVFTDKVDISSQISSMNQSLQQSKDYIKEAQRLLDTVNPSLISMLS | |
| | MIILYVLSIASLCIGLITFISFIIVEKKRNTYSRLEDRRVRPTSSGDLYYIGT | |
| 41 | ILHY EKLSKIGLVK GVTRKYKIKS NPLTKDIVIK MIPNVSNMSQ | Nipah virus NiV- |
| | CTGSVMENYK TRLNGILTPI KGALEIYKNN THDLVGDVRL AGVIMAGVAI | F F0 (aa 27-546) |
| | GIATAAQITA GVALYEAMKN ADNINKLKSS IESTNEAVVK LQETAEKTVY | |
| | VLTALQDYIN TNLVPTIDKI SCKQTELSLD LALSKYLSDL LFVFGPNLQD | |
| | PVSNSMTIQA ISQAFGGNYE TLLRTLGYAT EDFDDLLESD SITGQIIYVD | |
| | LSSYYIIVRV YFPILTEIQQ AYIQELLPVS FNNDNSEWIS IVPNFILVRN | |
| | TLISNIEIGF CLITKRSVIC NQDYATPMTN NMRECLTGST EKCPRELVVS | |
| | SHVPRFALSN GVLFANCISV TCQCQTTGRA ISQSGEQTLL MIDNTTCPTA | |
| | VLGNVIISLG KYLGSVNYNS EGIAIGPPVF TDKVDISSQI SSMNQSLQQS | |
| | KDYIKEAQRL LDTVNPSLIS MLSMIILYVL SIASLCIGLI TFISFIIVEK | |
| | KRNTYSRLED RRVRPTSSGD LYYIGT | |
| 42 | MKKINEGLLDSKILSA FNTVIALLGS IVIIVMNIMI IQNYTRSTDN | NiVG protein |
| | QAVIKDALQG IQQQIKGLAD KIGTEIGPKV SLIDTSSTIT IPANIGLLGS | attachment |
| | KISQSTASIN ENVNEKCKFT LPPLKIHECN ISCPNPLPFR EYRPQTEGVS | glycoprotein |
| | NLVGLPNNIC LQKTSNQILK PKLISYTLPV VGQSGTCITD PLLAMDEGYF | Truncated (Gc Δ |
| | AYSHLERIGS CSRGVSKQRI IGVGEVLDRG DEVPSLFMTN VWTPPNPNTV | 34) |
| | YHCSAVYNNE FYYVLCAVST VGDPILNSTY WSGSLMMTRL AVKPKSNGGG | |
| | YNQHQLALRS IEKGRYDKVM PYGPSGIKQG DTLYFPAVGF LVRTEFKYND | |
| | SNCPITKCQY SKPENCRLSM GIRPNSHYIL RSGLLKYNLS DGENPKVVFI | |
| | EISDQRLSIG SPSKIYDSLG QPVFYQASFS WDTMIKFGDV LTVNPLVVNW | |
| | RNNTVISRPG QSQCPRENTC PEICWEGVYN DAFLIDRINW ISAGVFLDSN | |
| | QTAENPVFTV FKDNEILYRA QLASEDTNAQ KTITNCFLLK NKIWCISLVE | |
| | IYDTGDNVIR PKLFAVKIPE QCT | |
| 43 | MTMDIKKINE GLLDSKILSA FNTVIALLGS IVIIVMNIMI IQNYTRSTDN | NiVG protein |
| | QAVIKDALQG IQQQIKGLAD KIGTEIGPKV SLIDTSSTIT IPANIGLLGS | attachment |
| | KISQSTASIN ENVNEKCKFT LPPLKIHECN ISCPNPLPFR EYRPQTEGVS | glycoprotein |
| | NLVGLPNNIC LQKTSNQILK PKLISYTLPV VGQSGTCITD PLLAMDEGYF | Truncated Δ30 |
| | AYSHLERIGS CSRGVSKQRI IGVGEVLDRG DEVPSLFMTN VWTPPNPNTV | |
| | YHCSAVYNNE FYYVLCAVST VGDPILNSTY WSGSLMMTRL AVKPKSNGGG | |
| | YNQHQLALRS IEKGRYDKVM PYGPSGIKQG DTLYFPAVGF LVRTEFKYND | |
| | SNCPITKCQY SKPENCRLSM GIRPNSHYIL RSGLLKYNLS DGENPKVVFI | |
| | EISDQRLSIG SPSKIYDSLG QPVFYQASFS WDTMIKFGDV LTVNPLVVNW | |
| | RNNTVISRPG QSQCPRENTC PEICWEGVYN DAFLIDRINW ISAGVFLDSN | |
| | QTAENPVFTV FKDNEILYRA QLASEDTNAQ KTITNCFLLK NKIWCISLVE | |
| | IYDTGDNVIR PKLFAVKIPE QCT | |
| 44 | MGNTTSDKGK IPSKVIKSYY GTMDIKKINE GLLDSKILSA FNTVIALLGS | NiVG protein |
| | IVIIVMNIMI IQNYTRSTDN QAVIKDALQG IQQQIKGLAD KIGTEIGPKV | attachment |
| | SLIDTSSTIT IPANIGLLGS KISQSTASIN ENVNEKCKFT LPPLKIHECN | glycoprotein |
| | ISCPNPLPFR EYRPQTEGVS NLVGLPNNIC LQKTSNQILK PKLISYTLPV | Truncated Δ10 |
| | VGQSGTCITD PLLAMDEGYF AYSHLERIGS CSRGVSKQRI IGVGEVLDRG | |
| | DEVPSLFMTN VWTPPNPNTV YHCSAVYNNE FYYVLCAVST VGDPILNSTY | |
| | WSGSLMMTRL AVKPKSNGGG YNQHQLALRS IEKGRYDKVM PYGPSGIKQG | |
| | DTLYFPAVGF LVRTEFKYND SNCPITKCQY SKPENCRLSM GIRPNSHYIL | |
| | RSGLLKYNLS DGENPKVVFI EISDQRLSIG SPSKIYDSLG QPVFYQASFS | |
| | WDTMIKFGDV LTVNPLVVNW RNNTVISRPG QSQCPRENTC PEICWEGVYN | |
| | DAFLIDRINW ISAGVFLDSN QTAENPVFTV FKDNEILYRA QLASEDTNAQ | |
| | KTITNCFLLK NKIWCISLVE IYDTGDNVIR PKLFAVKIPE QC | |
| 45 | MGKGK IPSKVIKSYY GTMDIKKINE GLLDSKILSA FNTVIALLGS | NiVG protein |
| | IVIIVMNIMI IQNYTRSTDN QAVIKDALQG IQQQIKGLAD KIGTEIGPKV | attachment |
| | SLIDTSSTIT IPANIGLLGS KISQSTASIN ENVNEKCKFT LPPLKIHECN | glycoprotein |
| | ISCPNPLPFR EYRPQTEGVS NLVGLPNNIC LQKTSNQILK PKLISYTLPV | Truncated Δ15 |
| | VGQSGTCITD PLLAMDEGYF AYSHLERIGS CSRGVSKQRI IGVGEVLDRG | |
| | DEVPSLFMTN VWTPPNPNTV YHCSAVYNNE FYYVLCAVST VGDPILNSTY | |
| | WSGSLMMTRL AVKPKSNGGG YNQHQLALRS IEKGRYDKVM PYGPSGIKQG | |
| | DTLYFPAVGF LVRTEFKYND SNCPITKCQY SKPENCRLSM GIRPNSHYIL | |
| | RSGLLKYNLS DGENPKVVFI EISDQRLSIG SPSKIYDSLG QPVFYQASES | |
| | WDTMIKFGDV LTVNPLVVNW RNNTVISRPG QSQCPRENTC PEICWEGVYN | |
| | DAFLIDRINW ISAGVFLDSN QTAENPVFTV FKDNEILYRA QLASEDTNAQ | |
| | KTITNCFLLK NKIWCISLVE IYDTGDNVIR PKLFAVKIPE QC | |
| 46 | LAGVIMAGVAIGIATAAQITAGVALYEAMKNADNINKLKSSIESTNEAVVKLQE | Nipah virus NiV |
| | TAEKTVYVLTALQDYINTNLVPTIDKISCKQTELSLDLALSKYLSDLLFVFGPNL | FF1 (aa 110- |
| | QDPVSNSMTIQAISQAFGGNYETLLRTLGYATEDFDDLLESDSITGQIIYVDLSS | 546) |
| | YYIIVRVYFPILTEIQQAYIQELLPVSFNNDNSEWISIVPNFILVRNTLISNIEIGFC | |
| | LITKRSVICNQDYATPMTNNMRECLTGSTEKCPRELVVSSHVPRFALSNGVLFA | |
| | NCISVTCQCQTTGRAISQSGEQTLLMIDNTTCPTAVLGNVIISLGKYLGSVNYN | |
| | SEGIAIGPPVFTDKVDISSQISSMNQSLQQSKDYIKEAQRLLDTVNPSLISMLSM | |
| | IILYVLSIASLCIGLITFISFIIVEKKRNTYSRLEDRRVRPTSSGDLYYIGT | |
| 47 | MALPVTALLLPLALLLHAARP | CD8α signal |
| | | peptide |
| 48 | METDTLLLWVLLLWVPGSTG | IgK signal |
| | | peptide |
| 49 | MLLLVTSLLLCELPHPAFLLIP | GMCSFR-α |
| | | (CSF2RA) |
| | | signal peptide |
| 50 | TTTPAPRPPTPAPTIASQPLSLRPEACRPAAGGAVHTRGLDFACD | CD8α hinge |
| | | domain |
| 51 | IEVMYPPPYLDNEKSNGTIIHVKGKHLCPSPLFPGPSKP | CD28 hinge |
| | | domain |
| 52 | AAAIEVMYPPPYLDNEKSNGTIIHVKGKHLCPSPLFPGPSKP | CD28 hinge |
| | | domain |
| 53 | ESKYGPPCPPCP | IgG4 hinge |
| | | domain |
| 54 | ESKYGPPCPSCP | IgG4 hinge |
| | | domain |
| 55 | ESKYGPPCPPCPAPEFLGGPSVFLFPPKPKDTLMISRTPEVTCVVVDVSQEDPEV | IgG4 hinge- |
| | QFNWYVDGVEVHNAKTKPREEQFNSTYRVVSVLTVLHQDWLNGKEYKCKV | CH2-CH3 |
| | SNKGLPSSIEKTISKAKGQPREPQVYTLPPSQEEMTKNQVSLTCLVKGFYPSDI | domain |
| | AVEWESNGQPENNYKTTPPVLDSDGSFFLYSRLTVDKSRWQEGNVFSCSVMH | |
| | EALHNHYTQKSLSLSLGK | |
| 56 | IYIWAPLAGTCGVLLLSLVITLYC | CD8α |
| | | transmembrane |
| | | domain |
| 57 | FWVLVVVGGVLACYSLLVTVAFIIFWV | CD28 |
| | | transmembrane |
| | | domain |
| 58 | MFWVLVVVGGVLACYSLLVTVAFIIFWV | CD28 |
| | | transmembrane |
| | | domain |
| 59 | KRGRKKLLYIFKQPFMRPVQTTQEEDGCSCRFPEEEEGGCEL | 4-1BB |
| | | costimulatory |
| | | domain |
| 60 | RSKRSRLLHSDYMNMTPRRPGPTRKHYQPYAPPRDFAAYRS | CD28 |
| | | costimulatory |
| | | domain |
| 61 | RVKFSRSADAPAYQQGQNQLYNELNLGRREEYDVLDKRRGRDPEMGGKPRR | CD3ζ signaling |
| | KNPQEGLYNELQKDKMAEAYSEIGMKGERRRGKGHDGLYQGLSTATKDTYD | domain |
| | ALHMQALPPR | |
| 62 | RVKFSRSADAPAYKQGQNQLYNELNLGRREEYDVLDKRRGRDPEMGGKPRR | CD3ζ signaling |
| | KNPQEGLYNELQKDKMAEAYSEIGMKGERRRGKGHDGLYQGLSTATKDTYD | domain (with Q |
| | ALHMQALPPR | to K mutation at |
| | | position 14) |
| 63 | DIQMTQTTSSLSASLGDRVTISCRASQDISKYLNWYQQKPDGTVKLLIYHTSRL | Anti-CD19 |
| | HSGVPSRFSGSGSGTDYSLTISNLEQEDIATYFCQQGNTLPYTFGGGTKLEITGS | FMC63 scFv |
| | TSGSGKPGSGEGSTKGEVKLQESGPGLVAPSQSLSVTCTVSGVSLPDYGVSWI | entire sequence, |
| | RQPPRKGLEWLGVIWGSETTYYNSALKSRLTIIKDNSKSQVFLKMNSLQTDDT | with Whitlow |
| | AIYYCAKHYYYGGSYAMDYWGQGTSVTVSS | linker |
| 64 | DIQMTQTTSSLSASLGDRVTISCRASQDISKYLNWYQQKPDGTVKLLIYHTSRL | Anti-CD19 |
| | HSGVPSRFSGSGSGTDYSLTISNLEQEDIATYFCQQGNTLPYTFGGGTKLEIT | FMC63 scFv |
| | | light chain |
| | | variable region |
| 65 | QDISKY | Anti-CD19 |
| | | FMC63 scFv |
| | | light chain |
| | | CDR1 |
| 66 | HTS | Anti-CD19 |
| | | FMC63 scFv |
| | | light chain |
| | | CDR2 |
| 67 | QQGNTLPYT | Anti-CD19 |
| | | FMC63 scFv |
| | | light chain |
| | | CDR3 |
| 68 | GSTSGSGKPGSGEGSTKG | Whitlow linker |
| 69 | EVKLQESGPGLVAPSQSLSVTCTVSGVSLPDYGVSWIRQPPRKGLEWLGVIWG | Anti-CD19 |
| | SETTYYNSALKSRLTIIKDNSKSQVFLKMNSLQTDDTAIYYCAKHYYYGGSYA | FMC63 scFv |
| | MDYWGQGTSVTVSS | heavy chain |
| | | variable region |
| 70 | GVSLPDYG | Anti-CD19 |
| | | FMC63 scFv |
| | | heavy chain |
| | | CDR1 |
| 71 | IWGSETT | Anti-CD19 |
| | | FMC63 scFv |
| | | heavy chain |
| | | CDR2 |
| 72 | AKHYYYGGSYAMDY | Anti-CD19 |
| | | FMC63 scFv |
| | | heavy chain |
| | | CDR3 |
| 73 | DIQMTQTTSSLSASLGDRVTISCRASQDISKYLNWYQQKPDGTVKLLIYHTSRL | Anti-CD19 |
| | HSGVPSRFSGSGSGTDYSLTISNLEQEDIATYFCQQGNTLPYTFGGGTKLEITGG | FMC63 scFv |
| | GGSGGGGSGGGGSEVKLQESGPGLVAPSQSLSVTCTVSGVSLPDYGVSWIRQP | entire sequence, |
| | PRKGLEWLGVIWGSETTYYNSALKSRLTIIKDNSKSQVFLKMNSLQTDDTAIY | with 3xG4S |
| | YCAKHYYYGGSYAMDYWGQGTSVTVSS | linker |
| 74 | atggccttaccagtgaccgccttgctcctgccgctggccttgctgctccacgccgccaggccggacatccagatgac | Exemplary |
| | acagactacatcctccctgtctgcctctctgggagacagagtcaccatcagttgcagggcaagtcaggacattagta | CD19 CAR |
| | aatatttaaattggtatcagcagaaaccagatggaactgttaaactcctgatctaccatacatcaagattacactca | nucleotide |
| | ggagtcccatcaaggttcagtggcagtgggtctggaacagattattctctcaccattagcaacctggagcaagaaga | sequence |
| | tattgccacttacttttgccaacagggtaatacgcttccgtacacgttcggaggggggaccaagctggagatcacag | |
| | gctccacctctggatccggcaagcccggatctggcgagggatccaccaagggcgaggtgaaactgcaggagtcagga | |
| | cctggcctggtggcgccctcacagagcctgtccgtcacatgcactgtctcaggggtctcattacccgactatggtgt | |
| | aagctggattcgccagcctccacgaaagggtctggagtggctgggagtaatatggggtagtgaaaccacatactata | |
| | attcagctctcaaatccagactgaccatcatcaaggacaactccaagagccaagttttcttaaaaatgaacagtctg | |
| | caaactgatgacacagccatttactactgtgccaaacattattactacggtggtagctatgctatggactactgggg | |
| | ccaaggaacctcagtcaccgtctcctcaaccacgacgccagcgccgcgaccaccaacaccggcgcccaccatcgcgt | |
| | cgcagcccctgtccctgcgcccagaggcgtgccggccagcggcggggggcgcagtgcacacgagggggctggacttc | |
| | ctgtcactggttatcaccctttactgcaaacggggcagaaagaaactcctgtatatattcaaacaaccatttatgag | |
| | acgcctgtgatatctacatctgggcgcccttggccgggacttgtggggtccttctccagtacaaactactcaagagg | |
| | aagatggctgtagctgccgatttccagaagaagaagaaggaggatgtgaactgagagtgaagttcagcaggagcgca | |
| | gacgcccccgcgtaccagcagggccagaaccagctctataacgagctcaatctaggacgaagagaggagtacgatgt | |
| | tttggacaagagacgtggccgggaccctgagatggggggaaagccgagaaggaagaaccctcaggaaggcctgtaca | |
| | atgaactgcagaaagataagatggcggaggcctacagtgagattgggatgaaaggcgagcgccggaggggcaagggg | |
| | cacgatggcctttaccagggtctcagtacagccaccaaggacacctacgacgcccttcacatgcaggccctgccccc | |
| | tcgc | |
| 75 | MALPVTALLLPLALLLHAARPDIQMTQTTSSLSASLGDRVTISCRASQDISKYL | Exemplary |
| | NWYQQKPDGTVKLLIYHTSRLHSGVPSRFSGSGSGTDYSLTISNLEQEDIATYF | CD19 CAR |
| | CQQGNTLPYTFGGGTKLEITGSTSGSGKPGSGEGSTKGEVKLQESGPGLVAPS | amino acid |
| | QSLSVTCTVSGVSLPDYGVSWIRQPPRKGLEWLGVIWGSETTYYNSALKSRLT | sequence |
| | IIKDNSKSQVFLKMNSLQTDDTAIYYCAKHYYYGGSYAMDYWGQGTSVTVSS | |
| | TTTPAPRPPTPAPTIASQPLSLRPEACRPAAGGAVHTRGLDFACDIYIWAPLAGT | |
| | CGVLLLSLVITLYCKRGRKKLLYIFKQPFMRPVQTTQEEDGCSCRFPEEEEGGC | |
| | ELRVKFSRSADAPAYQQGQNQLYNELNLGRREEYDVLDKRRGRDPEMGGKP | |
| | RRKNPQEGLYNELQKDKMAEAYSEIGMKGERRRGKGHDGLYQGLSTATKDT | |
| | YDALHMQALPPR | |
| 76 | atggccttaccagtgaccgccttgctcctgccgctggccttgctgctccacgccgccaggccggacatccagatgac | Tisagenlecleucel |
| | acagactacatcctccctgtctgcctctctgggagacagagtcaccatcagttgcagggcaagtcaggacattagta | CD19 CAR |
| | aatatttaaattggtatcagcagaaaccagatggaactgttaaactcctgatctaccatacatcaagattacactca | nucleotide |
| | ggagtcccatcaaggttcagtggcagtgggtctggaacagattattctctcaccattagcaacctggagcaagaaga | sequence |
| | tattgccacttacttttgccaacagggtaatacgcttccgtacacgttcggaggggggaccaagctggagatcacag | |
| | gtggcggtggctcgggcggtggtgggtcgggtggcggcggatctgaggtgaaactgcaggagtcaggacctggcctg | |
| | gtggcgccctcacagagcctgtccgtcacatgcactgtctcaggggtctcattacccgactatggtgtaagctggat | |
| | tcgccagcctccacgaaagggtctggagtggctgggagtaatatggggtagtgaaaccacatactataattcagctc | |
| | tcaaatccagactgaccatcatcaaggacaactccaagagccaagttttcttaaaaatgaacagtctgcaaactgat | |
| | gacacagccatttactactgtgccaaacattattactacggtggtagctatgctatggactactggggccaaggaac | |
| | ctcagtcaccgtctcctcaaccacgacgccagcgccgcgaccaccaacaccggcgcccaccatcgcgtcgcagcccc | |
| | tgtccctgcgcccagaggcgtgccggccagcggcggggggcgcagtgcacacgagggggctggacttcgcctgtgat | |
| | atctacatctgggcgcccttggccgggacttgtggggtccttctcctgtcactggttatcaccctttactgcaaacg | |
| | gggcagaaagaaactcctgtatatattcaaacaaccatttatgagaccagtacaaactactcaagaggaagatggct | |
| | gtagctgccgatttccagaagaagaagaaggaggatgtgaactgagagtgaagttcagcaggagcgcagacgccccc | |
| | gcgtacaagcagggccagaaccagctctataacgagctcaatctaggacgaagagaggagtacgatgttttggacaa | |
| | gagacgtggccgggaccctgagatggggggaaagccgagaaggaagaaccctcaggaaggcctgtacaatgaactgc | |
| | agaaagataagatggcggaggcctacagtgagattgggatgaaaggcgagcgccggaggggcaaggggcacgatggc | |
| | ctttaccagggtctcagtacagccaccaaggacacctacgacgcccttcacatgcaggccctgccccctcgc | |
| 77 | MALPVTALLLPLALLLHAARPDIQMTQTTSSLSASLGDRVTISCRASQDISKYL | Tisagenlecleucel |
| | NWYQQKPDGTVKLLIYHTSRLHSGVPSRFSGSGSGTDYSLTISNLEQEDIATYF | CD19 CAR |
| | CQQGNTLPYTFGGGTKLEITGGGGSGGGGSGGGGSEVKLQESGPGLVAPSQSL | amino acid |
| | SVTCTVSGVSLPDYGVSWIRQPPRKGLEWLGVIWGSETTYYNSALKSRLTIIK | sequence |
| | DNSKSQVFLKMNSLQTDDTAIYYCAKHYYYGGSYAMDYWGQGTSVTVSSTT | |
| | TPAPRPPTPAPTIASQPLSLRPEACRPAAGGAVHTRGLDFACDIYIWAPLAGTCG | |
| | VLLLSLVITLYCKRGRKKLLYIFKQPFMRPVQTTQEEDGCSCRFPEEEEGGCEL | |
| | RVKFSRSADAPAYKQGQNQLYNELNLGRREEYDVLDKRRGRDPEMGGKPRR | |
| | KNPQEGLYNELQKDKMAEAYSEIGMKGERRRGKGHDGLYQGLSTATKDTYD | |
| | ALHMQALPPR | |
| 78 | atgctgctgctggtgaccagcctgctgctgtgcgagctgccccaccccgcctttctgctgatccccgacatccagat | Lisocabtagene |
| | gacccagaccacctccagcctgagcgccagcctgggcgaccgggtgaccatcagctgccgggccagccaggacatca | maraleucel |
| | gcaagtacctgaactggtatcagcagaagcccgacggcaccgtcaagctgctgatctaccacaccagccggctgcac | CD19 CAR |
| | agcggcgtgcccagccggtttagcggcagcggctccggcaccgactacagcctgaccatctccaacctggaacagga | nucleotide |
| | agatatcgccacctacttttgccagcagggcaacacactgccctacacctttggcggcggaacaaagctggaaatca | sequence |
| | ccggcagcacctccggcagcggcaagcctggcagcggcgagggcagcaccaagggcgaggtgaagctgcaggaaagc | |
| | ggccctggcctggtggcccccagccagagcctgagcgtgacctgcaccgtgagcggcgtgagcctgcccgactacgg | |
| | cgtgagctggatccggcagccccccaggaagggcctggaatggctgggcgtgatctggggcagcgagaccacctact | |
| | acaacagcgccctgaagagccggctgaccatcatcaaggacaacagcaagagccaggtgttcctgaagatgaacagc | |
| | ctgcagaccgacgacaccgccatctactactgcgccaagcactactactacggcggcagctacgccatggactactg | |
| | gggccagggcaccagcgtgaccgtgagcagcgaatctaagtacggaccgccctgccccccttgccctatgttctggg | |
| | tgctggtggtggtcggaggcgtgctggcctgctacagcctgctggtcaccgtggccttcatcatcttttgggtgaaa | |
| | cggggcagaaagaaactcctgtatatattcaaacaaccatttatgagaccagtacaaactactcaagaggaagatgg | |
| | ctgtagctgccgatttccagaagaagaagaaggaggatgtgaactgcgggtgaagttcagcagaagcgccgacgccc | |
| | ctgcctaccagcagggccagaatcagctgtacaacgagctgaacctgggcagaagggaagagtacgacgtcctggat | |
| | aagcggagaggccgggaccctgagatgggcggcaagcctcggcggaagaacccccaggaaggcctgtataacgaact | |
| | gcagaaagacaagatggccgaggcctacagcgagatcggcatgaagggcgagcggaggcggggcaagggccacgacg | |
| | gcctgtatcagggcctgtccaccgccaccaaggatacctacgacgccctgcacatgcaggccctgcccccaagg | |
| 79 | MLLLVTSLLLCELPHPAFLLIPDIQMTQTTSSLSASLGDRVTISCRASQDISKYLN | Lisocabtagene |
| | WYQQKPDGTVKLLIYHTSRLHSGVPSRFSGSGSGTDYSLTISNLEQEDIATYFC | maraleucel |
| | QQGNTLPYTFGGGTKLEITGSTSGSGKPGSGEGSTKGEVKLQESGPGLVAPSQS | CD19 CAR |
| | LSVTCTVSGVSLPDYGVSWIRQPPRKGLEWLGVIWGSETTYYNSALKSRLTIIK | amino acid |
| | DNSKSQVFLKMNSLQTDDTAIYYCAKHYYYGGSYAMDYWGQGTSVTVSSES | sequence |
| | KYGPPCPPCPMFWVLVVVGGVLACYSLLVTVAFIIFWVKRGRKKLLYIFKQPF | |
| | MRPVQTTQEEDGCSCRFPEEEEGGCELRVKFSRSADAPAYQQGQNQLYNELN | |
| | LGRREEYDVLDKRRGRDPEMGGKPRRKNPQEGLYNELQKDKMAEAYSEIGM | |
| | KGERRRGKGHDGLYQGLSTATKDTYDALHMQALPPR | |
| 80 | atgcttctcctggtgacaagccttctgctctgtgagttaccacacccagcattcctcctgatcccagacatccagat | Axicabtagene |
| | gacacagactacatcctccctgtctgcctctctgggagacagagtcaccatcagttgcagggcaagtcaggacatta | ciloleucel CD19 |
| | gtaaatatttaaattggtatcagcagaaaccagatggaactgttaaactcctgattaccatacatcaagattacact | CAR nucleotide |
| | caggagtcccatcaaggttcagtggcagtgggtctggaacagattattctctcaccattagcaacctggagcaagaa | sequence |
| | gatattgccacttacttttgccaacagggtaatacgcttccgtacacgttcggaggggggactaagttggaaataac | |
| | aggctccacctctggatccggcaagcccggatctggcgagggatccaccaagggcgaggtgaaactgcaggagtcag | |
| | gacctggcctggtggcgccctcacagagcctgtccgtcacatgcactgtctcaggggtctcattacccgactatggt | |
| | gtaagctggattcgccagcctccacgaaagggtctggagtggctgggagtaatatggggtagtgaaaccacatacta | |
| | taattcagctctcaaatccagactgaccatcatcaaggacaactccaagagccaagttttcttaaaaatgaacagtc | |
| | tgcaaactgatgacacagccatttactactgtgccaaacattattactacggtggtagctatgctatggactactgg | |
| | ggtcaaggaacctcagtcaccgtctcctcagcggccgcaattgaagttatgtatcctcctccttacctagacaatga | |
| | gaagagcaatggaaccattatccatgtgaaagggaaacacctttgtccaagtcccctatttcccggaccttctaagc | |
| | ccttttgggtgctggtggtggttgggggagtcctggcttgctatagcttgctagtaacagtggcctttattattttc | |
| | tgggtgaggagtaagaggagcaggctcctgcacagtgactacatgaacatgactccccgccgccccgggcccacccg | |
| | caagcattaccagccctatgccccaccacgcgacttcgcagcctatcgctccagagtgaagttcagcaggagcgcag | |
| | acgcccccgcgtaccagcagggccagaaccagctctataacgagctcaatctaggacgaagagaggagtacgatgtt | |
| | ttggacaagagacgtggccgggaccctgagatggggggaaagccgagaaggaagaaccctcaggaaggcctgtacaa | |
| | tgaactgcagaaagataagatggcggaggcctacagtgagattgggatgaaaggcgagcgccggaggggcaaggggc | |
| | acgatggcctttaccagggtctcagtacagccaccaaggacacctacgacgcccttcacatgcaggccctgccccct | |
| | cgc | |
| 81 | MLLLVTSLLLCELPHPAFLLIPDIQMTQTTSSLSASLGDRVTISCRASQDISKYLN | Axicabtagene |
| | WYQQKPDGTVKLLIYHTSRLHSGVPSRFSGSGSGTDYSLTISNLEQEDIATYFC | ciloleucel CD19 |
| | QQGNTLPYTFGGGTKLEITGSTSGSGKPGSGEGSTKGEVKLQESGPGLVAPSQS | CAR amino acid |
| | LSVTCTVSGVSLPDYGVSWIRQPPRKGLEWLGVIWGSETTYYNSALKSRLTIIK | sequence |
| | DNSKSQVFLKMNSLQTDDTAIYYCAKHYYYGGSYAMDYWGQGTSVTVSSAA | |
| | AIEVMYPPPYLDNEKSNGTIIHVKGKHLCPSPLFPGPSKPFWVLVVVGGVLAC | |
| | YSLLVTVAFIIFWVRSKRSRLLHSDYMNMTPRRPGPTRKHYQPYAPPRDFAAY | |
| | RSRVKFSRSADAPAYQQGQNQLYNELNLGRREEYDVLDKRRGRDPEMGGKP | |
| | RRKNPQEGLYNELQKDKMAEAYSEIGMKGERRRGKGHDGLYQGLSTATKDT | |
| | YDALHMQALPPR | |
| 82 | DIVLTQSPAILSASPGEKVTMTCRASSSVNYMDWYQKKPGSSPKPWIYATSNL | Anti-CD20 |
| | ASGVPARFSGSGSGTSYSLTISRVEAEDAATYYCQQWSFNPPTFGGGTKLEIKG | Leu16 scFv |
| | STSGSGKPGSGEGSTKGEVQLQQSGAELVKPGASVKMSCKASGYTFTSYNMH | entire sequence, |
| | WVKQTPGQGLEWIGAIYPGNGDTSYNQKFKGKATLTADKSSSTAYMQLSSLTS | with Whitlow |
| | EDSADYYCARSNYYGSSYWFFDVWGAGTTVTVSS | linker |
| 83 | DIVLTQSPAILSASPGEKVTMTCRASSSVNYMDWYQKKPGSSPKPWIYATSNL | Anti-CD20 |
| | ASGVPARFSGSGSGTSYSLTISRVEAEDAATYYCQQWSFNPPTFGGGTKLEIK | Leu16 scFv |
| | | light chain |
| | | variable region |
| 84 | RASSSVNYMD | Anti-CD20 |
| | | Leu16 scFv |
| | | light chain |
| | | CDR1 |
| 85 | ATSNLAS | Anti-CD20 |
| | | Leu16 scFv |
| | | light chain |
| | | CDR2 |
| 86 | QQWSFNPPT | Anti-CD20 |
| | | Leu16 scFv |
| | | light chain |
| | | CDR3 |
| 87 | EVQLQQSGAELVKPGASVKMSCKASGYTFTSYNMHWVKQTPGQGLEWIGAI | Anti-CD20 |
| | YPGNGDTSYNQKFKGKATLTADKSSSTAYMQLSSLTSEDSADYYCARSNYYG | Leu16 scFv |
| | SSYWFFDVWGAGTTVTVSS | heavy chain |
| 88 | SYNMH | Anti-CD20 |
| | | Leu16 scFv |
| | | heavy chain |
| | | CDR1 |
| 89 | AIYPGNGDTSYNQKFKG | Anti-CD20 |
| | | Leu16 scFv |
| | | heavy chain |
| | | CDR2 |
| 90 | QVQLQQSGPGLVKPSQTLSLTCAISGDSVSSNSAAWNWIRQSPSRGLEWLGRT | Anti-CD22 |
| | YYRSKWYNDYAVSVKSRITINPDTSKNQFSLQLNSVTPEDTAVYYCAREVTGD | m971 scFv |
| | LEDAFDIWGQGTMVTVSSGGGGSGGGGSGGGGSDIQMTQSPSSLSASVGDRV | entire sequence, |
| | TITCRASQTIWSYLNWYQQRPGKAPNLLIYAASSLQSGVPSRFSGRGSGTDFTL | with 3xG4S |
| | TISSLQAEDFATYYCQQSYSIPQTFGQGTKLEIK | linker |
| 91 | QVQLQQSGPGLVKPSQTLSLTCAISGDSVSSNSAAWNWIRQSPSRGLEWLGRT | Anti-CD22 |
| | YYRSKWYNDYAVSVKSRITINPDTSKNQFSLQLNSVTPEDTAVYYCAREVTGD | m971 scFv |
| | LEDAFDIWGQGTMVTVSS | heavy chain |
| | | variable region |
| 92 | GDSVSSNSAA | Anti-CD22 |
| | | m971 scFv |
| | | heavy chain |
| | | CDR1 |
| 93 | TYYRSKWYN | Anti-CD22 |
| | | m971 scFv |
| | | heavy chain |
| | | CDR2 |
| 94 | AREVTGDLEDAFDI | Anti-CD22 |
| | | m971 scFv |
| | | heavy chain |
| | | CDR3 |
| 95 | DIQMTQSPSSLSASVGDRVTITCRASQTIWSYLNWYQQRPGKAPNLLIYAASSL | Anti-CD22 |
| | QSGVPSRFSGRGSGTDFTLTISSLQAEDFATYYCQQSYSIPQTFGQGTKLEIK | m971 scFv light |
| | | chain |
| 96 | QTIWSY | Anti-CD22 |
| | | m971 scFv light |
| | | chain CDR1 |
| 97 | AAS | Anti-CD22 |
| | | m971 scFv light |
| | | chain CDR2 |
| 98 | QQSYSIPQT | Anti-CD22 |
| | | m971 scFv light |
| | | chain CDR3 |
| 99 | QVQLQQSGPGMVKPSQTLSLTCAISGDSVSSNSVAWNWIRQSPSRGLEWLGRT | Anti-CD22 |
| | YYRSTWYNDYAVSMKSRITINPDTNKNQFSLQLNSVTPEDTAVYYCAREVTG | m971-L7 scFv |
| | DLEDAFDIWGQGTMVTVSSGGGGSGGGGSGGGGSDIQMIQSPSSLSASVGDR | entire sequence, |
| | VTITCRASQTIWSYLNWYRQRPGEAPNLLIYAASSLQSGVPSRFSGRGSGTDFT | with 3xG4S |
| | LTISSLQAEDFATYYCQQSYSIPQTFGQGTKLEIK | linker |
| 100 | QVQLQQSGPGMVKPSQTLSLTCAISGDSVSSNSVAWNWIRQSPSRGLEWLGRT | Anti-CD22 |
| | YYRSTWYNDYAVSMKSRITINPDTNKNQFSLQLNSVTPEDTAVYYCAREVTG | m971-L7 scFv |
| | DLEDAFDIWGQGTMVTVSS | heavy chain |
| | | variable region |
| 101 | GDSVSSNSVA | Anti-CD22 |
| | | m971-L7 scFv |
| | | heavy chain |
| | | CDR1 |
| 102 | TYYRSTWYN | Anti-CD22 |
| | | m971-L7 scFv |
| | | heavy chain |
| | | CDR2 |
| 103 | AREVTGDLEDAFDI | Anti-CD22 |
| | | m971-L7 scFv |
| | | heavy chain |
| | | CDR3 |
| 104 | DIQMIQSPSSLSASVGDRVTITCRASQTIWSYLNWYRQRPGEAPNLLIYAASSL | Anti-CD22 |
| | QSGVPSRFSGRGSGTDFTLTISSLQAEDFATYYCQQSYSIPQTFGQGTKLEIK | m971-L7 scFv |
| | | light chain |
| | | variable region |
| 105 | QTIWSY | Anti-CD22 |
| | | m971-L7 scFv |
| | | light chain |
| | | CDR1 |
| 106 | AAS | Anti-CD22 |
| | | m971-L7 scFv |
| | | light chain |
| | | CDR2 |
| 107 | QQSYSIPQT | Anti-CD22 |
| | | m971-L7 scFv |
| | | light chain |
| | | CDR3 |
| 108 | DIVLTQSPASLAMSLGKRATISCRASESVSVIGAHLIHWYQQKPGQPPKLLIYL | Anti-BCMA |
| | ASNLETGVPARFSGSGSGTDFTLTIDPVEEDDVAIYSCLQSRIFPRTFGGGTKLEI | C11D5.3 scFv |
| | KGSTSGSGKPGSGEGSTKGQIQLVQSGPELKKPGETVKISCKASGYTFTDYSIN | entire sequence, |
| | WVKRAPGKGLKWMGWINTETREPAYAYDFRGRFAFSLETSASTAYLQINNLK | with Whitlow |
| | YEDTATYFCALDYSYAMDYWGQGTSVTVSS | linker |
| 109 | DIVLTQSPASLAMSLGKRATISCRASESVSVIGAHLIHWYQQKPGQPPKLLIYL | Anti-BCMA |
| | ASNLETGVPARFSGSGSGTDFTLTIDPVEEDDVAIYSCLQSRIFPRTFGGGTKLEI | C11D5.3 scFv |
| | K | light chain |
| | | variable region |
| 110 | RASESVSVIGAHLIH | Anti-BCMA |
| | | C11D5.3 scFv |
| | | light chain |
| | | CDR1 |
| 111 | LASNLET | Anti-BCMA |
| | | C11D5.3 scFv |
| | | light chain |
| | | CDR2 |
| 112 | LQSRIFPRT | Anti-BCMA |
| | | C11D5.3 scFv |
| | | light chain |
| | | CDR3 |
| 113 | QIQLVQSGPELKKPGETVKISCKASGYTFTDYSINWVKRAPGKGLKWMGWIN | Anti-BCMA |
| | TETREPAYAYDERGRFAFSLETSASTAYLQINNLKYEDTATYFCALDYSYAMDY | C11D5.3 scFv |
| | WGQGTSVTVSS | heavy chain |
| | | variable region |
| 114 | DYSIN | Anti-BCMA |
| | | C11D5.3 scFv |
| | | heavy chain |
| | | CDR1 |
| 115 | WINTETREPAYAYDFRG | Anti-BCMA |
| | | C11D5.3 scFv |
| | | heavy chain |
| | | CDR2 |
| 116 | DYSYAMDY | Anti-BCMA |
| | | C11D5.3 scFv |
| | | heavy chain |
| | | CDR3 |
| 117 | DIVLTQSPPSLAMSLGKRATISCRASESVTILGSHLIYWYQQKPGQPPTLLIQLA | Anti-BCMA |
| | SNVQTGVPARFSGSGSRTDFTLTIDPVEEDDVAVYYCLQSRTIPRTFGGGTKLEI | C12A3.2 scFv |
| | KGSTSGSGKPGSGEGSTKGQIQLVQSGPELKKPGETVKISCKASGYTFRHYSM | entire sequence, |
| | NWVKQAPGKGLKWMGRINTESGVPIYADDFKGRFAFSVETSASTAYLVINNL | with Whitlow |
| | KDEDTASYFCSNDYLYSLDFWGQGTALTVSS | linker |
| 118 | DIVLTQSPPSLAMSLGKRATISCRASESVTILGSHLIYWYQQKPGQPPTLLIQLA | Anti-BCMA |
| | SNVQTGVPARFSGSGSRTDFTLTIDPVEEDDVAVYYCLQSRTIPRTFGGGTKLEI | C12A3.2 scFv |
| | K | light chain |
| | | variable region |
| 119 | RASESVTILGSHLIY | Anti-BCMA |
| | | C12A3.2 scFv |
| | | light chain |
| | | CDR1 |
| 120 | LASNVQT | Anti-BCMA |
| | | C12A3.2 scFv |
| | | light chain |
| | | CDR2 |
| 121 | LQSRTIPRT | Anti-BCMA |
| | | C12A3.2 scFv |
| | | light chain |
| | | CDR3 |
| 122 | QIQLVQSGPELKKPGETVKISCKASGYTFRHYSMNWVKQAPGKGLKWMGRI | Anti-BCMA |
| | NTESGVPIYADDFKGRFAFSVETSASTAYLVINNLKDEDTASYFCSNDYLYSLD | C12A3.2 scFv |
| | FWGQGTALTVSS | heavy chain |
| | | variable region |
| 123 | HYSMN | Anti-BCMA |
| | | C12A3.2 scFv |
| | | heavy chain |
| | | CDR1 |
| 124 | RINTESGVPIYADDFKG | Anti-BCMA |
| | | C12A3.2 scFv |
| | | heavy chain |
| | | CDR2 |
| 125 | DYLYSLDF | Anti-BCMA |
| | | C12A3.2 scFv |
| | | heavy chain |
| | | CDR3 |
| 126 | EVQLLESGGGLVQPGGSLRLSCAASGFTFSSYAMSWVRQAPGKGLEWVSSISG | Anti-BCMA |
| | SGDYIYYADSVKGRFTISRDISKNTLYLQMNSLRAEDTAVYYCAKEGTGANSS | FHVH33 entire |
| | LADYRGQGTLVTVSS | sequence |
| 127 | GFTFSSYA | Anti-BCMA |
| | | FHVH33 CDR1 |
| 128 | ISGSGDYI | Anti-BCMA |
| | | FHVH33 CDR2 |
| 129 | AKEGTGANSSLADY | Anti-BCMA |
| | | FHVH33 CDR3 |
| 130 | DIQMTQSPSSLSASVGDRVTITCRASQSISSYLNWYQQKPGKAPKLLIYAASSL | Anti-BCMA |
| | QSGVPSRFSGSGSGTDFTLTISSLQPEDFATYYCQQKYDLLTFGGGTKVEIKGST | CT103A scFv |
| | SGSGKPGSGEGSTKGQLQLQESGPGLVKPSETLSLTCTVSGGSISSSSYYWGWI | entire sequence, |
| | RQPPGKGLEWIGSISYSGSTYYNPSLKSRVTISVDTSKNQFSLKLSSVTAADTAV | with Whitlow |
| | YYCARDRGDTILDVWGQGTMVTVSS | linker |
| 131 | DIQMTQSPSSLSASVGDRVTITCRASQSISSYLNWYQQKPGKAPKLLIYAASSL | Anti-BCMA |
| | QSGVPSRFSGSGSGTDFTLTISSLQPEDFATYYCQQKYDLLTFGGGTKVEIK | CT103A scFv |
| | | light chain |
| | | variable region |
| 132 | QSISSY | Anti-BCMA |
| | | CT103A scFv |
| | | light chain |
| | | CDR1 |
| 133 | AAS | Anti-BCMA |
| | | CT103A scFv |
| | | light chain |
| | | CDR2 |
| 134 | QQKYDLLT | Anti-BCMA |
| | | CT103A scFv |
| | | light chain |
| | | CDR3 |
| 135 | QLQLQESGPGLVKPSETLSLTCTVSGGSISSSSYYWGWIRQPPGKGLEWIGSISY | Anti-BCMA |
| | SGSTYYNPSLKSRVTISVDTSKNQFSLKLSSVTAADTAVYYCARDRGDTILDV | CT103A scFv |
| | WGQGTMVTVSS | heavy chain |
| | | variable region |
| 136 | GGSISSSSYY | Anti-BCMA |
| | | CT103A scFv |
| | | heavy chain |
| | | CDR1 |
| 137 | ISYSGST | Anti-BCMA |
| | | CT103A scFv |
| | | heavy chain |
| | | CDR2 |
| 138 | ARDRGDTILDV | Anti-BCMA |
| | | CT103A scFv |
| | | heavy chain |
| | | CDR3 |
| 139 | atggccttaccagtgaccgccttgctcctgccgctggccttgctgctccacgccgccaggccggacatccagatgac | Exemplary |
| | ccagtctccatcctccctgtctgcatctgtaggagacagagtcaccatcacttgccgggcaagtcagagcattagca | BCMA CAR |
| | gctatttaaattggtatcagcagaaaccagggaaagcccctaagctcctgatctatgctgcatccagtttgcaaagt | nucleotide |
| | ggggtcccatcaaggttcagtggcagtggatctgggacagatttcactctcaccatcagcagtctgcaacctgaaga | sequence |
| | ttttgcaacttactactgtcagcaaaaatacgacctcctcacttttggggagggaccaaggttgagatcaaaggcag | |
| | caccagcggctccggcaagcctggctctggcgagggcagcacaaagggacagctgcagctgcaggagtcgggcccag | |
| | gactggtgaagccttcggagaccctgtccctcacctgcactgtctctggtggctccatcagcagtagtagttactac | |
| | tggggctggatccgccagcccccagggaaggggctggagtggattgggagtatctcctatagtgggagcacctacta | |
| | caacccgtccctcaagagtcgagtcaccatatccgtagacacgtccaagaaccagttctccctgaagctgagttctg | |
| | tgaccgccgcagacacggcggtgtactactgcgccagagatcgtggagacaccatactagacgtatggggtcagggt | |
| | acaatggtcaccgtcagctcattcgtgcccgtgttcctgcccgccaaacctaccaccacccctgcccctagacctcc | |
| | caccccagccccaacaatcgccagccagcctctgtctctgcggcccgaagcctgtagacctgctgccggcggagccg | |
| | tgcacaccagaggcctggacttcgcctgcgacatctacatctgggcccctctggccggcacctgtggcgtgctgctg | |
| | ctgagcctggtgatcaccctgtactgcaaccaccggaacaaacggggcagaaagaaactcctgtatatattcaaaca | |
| | accatttatgagaccagtacaaactactcaagaggaagatggctgtagctgccgatttccagaagaagaagaaggag | |
| | gatgtgaactgagagtgaagttcagcagatccgccgacgcccctgcctaccagcagggacagaaccagctgtacaac | |
| | gagctgaacctgggcagacgggaagagtacgacgtgctggacaagcggagaggccgggaccccgagatgggggaaag | |
| | cccagacggaagaacccccaggaaggcctgtataacgaactgcagaaagacaagatggccgaggcctacagcgagat | |
| | cggcatgaagggcgagcggaggcgcggcaagggccacgatggcctgtaccagggcctgagcaccgccaccaaggaca | |
| | cctacgacgccctgcacatgcaggccctgccccccaga | |
| 140 | MALPVTALLLPLALLLHAARPDIQMTQSPSSLSASVGDRVTITCRASQSISSYL | Exemplary |
| | NWYQQKPGKAPKLLIYAASSLQSGVPSRFSGSGSGTDFTLTISSLQPEDFATYY | BCMA CAR |
| | CQQKYDLLTFGGGTKVEIKGSTSGSGKPGSGEGSTKGQLQLQESGPGLVKPSE | amino acid |
| | TLSLTCTVSGGSISSSSYYWGWIRQPPGKGLEWIGSISYSGSTYYNPSLKSRVTI | sequence |
| | SVDTSKNQFSLKLSSVTAADTAVYYCARDRGDTILDVWGQGTMVTVSSFVPV | |
| | FLPAKPTTTPAPRPPTPAPTIASQPLSLRPEACRPAAGGAVHTRGLDFACDIYIW | |
| | APLAGTCGVLLLSLVITLYCNHRNKRGRKKLLYIFKQPFMRPVQTTQEEDGCS | |
| | CRFPEEEEGGCELRVKFSRSADAPAYQQGQNQLYNELNLGRREEYDVLDKRR | |
| | GRDPEMGGKPRRKNPQEGLYNELQKDKMAEAYSEIGMKGERRRGKGHDGLY | |
| | QGLSTATKDTYDALHMQALPPR | |
| 141 | ACRPAAGGAVHTRGLDFACDIYIWAPLAGTCGVLLLSLVITLYC | CD8 |
| | | Transmembrane |
| 142 | TTTPAPRPPTPAPTIASQPLSLRPE | CD8 Hinge |
| 143 | GGGGSGGGGSGGGGS | linker |
| 144 | SNYYGSSYWFFDV | Anti-CD20 |
| | | Leu16 scFv |
| | | heavy chain |
| | | CDR3 |
| 145 | RYTMH | CD3 CDR-H1 |
| 146 | YINPSRGYTNYNQKFKD | CD3 CDR-H2 |
| 147 | YYDDHYCLDY | CD3 CDR-H3 |
| 148 | RASSSVSYMN | CD3 CDR-L1 |
| 149 | DTSKVAS | CD3 CDR-L2 |
| 150 | QQWSSNPLT | CD3 CDR-L3 |
| 151 | DIKLQQSGAELARPGASVKMSCKTSGYTFTRYTMHWVKQRPGQGLEWIGYIN | CD3 VH |
| | PSRGYTNYNQKFKDKATLTTDKSSSTAYMQLSSLTSEDSAVYYCARYYDDHY | |
| | CLDYWGQGTTLTVSSVE | |
| 152 | DIQLTQSPAIMSASPGEKVTMTCRASSSVSYMNWYQQKSGTSPKRWIYDTSKV | CD3 VL |
| | ASGVPYRFSGSGSGTSYSLTISSMEAEDAATYYCQQWSSNPLTFGAGTKLELK | |
| 153 | DIKLQQSGAELARPGASVKMSCKTSGYTFTRYTMHWVKQRPGQGLEWIGYIN | CD3 scFv |
| | PSRGYTNYNQKFKDKATLTTDKSSSTAYMQLSSLTSEDSAVYYCARYYDDHY | |
| | CLDYWGQGTTLTVSSVEGGSGGSGGSGGSGGVDDIQLTQSPAIMSASPGEKVT | |
| | MTCRASSSVSYMNWYQQKSGTSPKRWIYDTSKVASGVPYRFSGSGSGTSYSL | |
| | TISSMEAEDAATYYCQQWSSNPLTFGAGTKLELK | |
| 154 | APTSSSTKKTQLQLEHLLLDLQMILNGINNYKNPKLTRMLTFKFYMPKKATEL | IL-2 (mature) |
| | KHLQCLEEELKPLEEVLNLAQSKNFHLRPRDLISNINVIVLELKGSETTFMCEY | |
| | ADETATIVEFLNRWITFCQSIISTLT | |
| 155 | MYRMQLLSCIALSLALVTNSAPTSSSTKKTQLQLEHLLLDLQMILNGINNYKN | IL-2 (precursor) |
| | PKLTRMLTFKFYMPKKATELKHLQCLEEELKPLEEVLNLAQSKNFHLRPRDLIS | |
| | NINVIVLELKGSETTFMCEYADETATIVEFLNRWITFCQSIISTLT | |
| 156 | PTSSSTKKTQLQLEHLLLDLQMILNGINNYKNPKLTRMLTFKFYMPKKATELK | Aldesleukin/ |
| | HLQCLEEELKPLEEVLNLAQSKNFHLRPRDLISNINVIVLELKGSETTFMCEYA | proleukin |
| | DETATIVEFLNRWITFSQSIISTLT | |
| 157 | GFTFSYYG | 17 |
| 158 | ISNDGSNK | 18 |
| 159 | RPEDTAVYYCAT | 19 |
| 160 | GYNFDTYW | 20 |
| 161 | IYPADSDT | 21 |
| 162 | ARFSSSAYDI | 22 |
| 163 | GYNFDTYW | 23 |
| 164 | IYPADSDT | 24 |
| 165 | SSAYDIARFS | 25 |
| 166 | GFTFNRYA | 26 |
| 167 | ISFDGGNQ | 27 |
| 168 | VRSPAGDWVAYFDY | 28 |
| 169 | ALPKRF | 29 |
| 170 | EDN | 30 |
| 171 | YSTDNTGGLWV | 31 |
| 172 | ALPRQF | 32 |
| 173 | KDT | 33 |
| 174 | DSSGTYHQSA | 34 |
| 175 | ALPRKF | 35 |
| 176 | QDT | 36 |
| 177 | QSADRSDSYV | 37 |
| 178 | EDISNH | 38 |
| 179 | DAS | 39 |
| 180 | HQYHDYPSYT | 40 |
| 181 | MFHVSFRYIF GLPPLILVLL PVASSDCDIE GKDGKQYESV LMVSIDQLLD | Genbank |
| | SMKEIGSNCL NNEFNFFKRH ICDANKEGMF LFRAARKLRQ FLKMNSTGDF | Accession No. |
| | DLHLLKVSEG TTILLNCTGQ VKGRKPAALG EAQPTKSLEE NKSLKEQKKL | P13232 (human |
| | NDLCFLKRLL QEIKTCWNKI LMGTKEH | IL-7) |
| 182 | MRISKPHLRSISIQCYLCLLLNSHFLTEAGIHVFILGCFSAGLPKTEANWVNVIS | IL-15 LSP |
| | DLKKIEDLIQSMHIDATLYTESDVHPSCKVTAMKCFLLELQVISLESGDASIHDT | |
| | VENLIILANNSLSSNGNVTESGCKECEELEEKNIKEFLQSFVHIVQMFINTS | |
| 183 | MVLGTIDLCSCFSAGLPKTEANWVNVISDLKKIEDLIQSMHIDATLYTESDVHP | IL-15 SSP |
| | SCKVTAMKCFLLELQVISLESGDASIHDTVENLIILANNSLSSNGNVTESGCKE | |
| | CEELEEKNIKEFLQSFVHIVQMFINTS | |
| 184 | NWVNVISDLKKIEDLIQSMHIDATLYTESDVHPSCKVTAMKCFLLELQVISLES | IL-15 mature |
| | GDASIHDTVENLIILANNSLSSNGNVTESGCKECEELEEKNIKEFLQSFVHIVQ | |
| | MFINTS | |
| 185 | QVQLVQSGAEVKKPGASVKVSCKAS | 24 |
| 186 | IHWVRQAPGQGLELIGW | 25 |
| 187 | NYAQKFQVKATMTRDTSISTAYMELSRLRSDDTAVYYC | 26 |
| 188 | WGQGTTVTVSS | 27 |
| 189 | DVVMTQSPLSLPVTLGQPASISCKSS | 28 |
| 190 | LNWLQQRPGQSPRRLI | 29 |
| 191 | KLDSGVPDRFSGSGSGTDFTLKISRVEAEDVGVYYC | 30 |
| 192 | FGQGTKLEIK | 31 |
| 193 | QVQLVQSGAEVKKPGASVKVSCKASGFNIK | 32 |
| 194 | WVRQAPGQGLELIG | 33 |
| 195 | KATMTRDTSISTAYMELSRLRSDDTAVYYCND | 34 |
| 196 | WGQGTTVTVSS | 35 |
| 197 | DVVMTQSPLSLPVTLGQPASISC | 36 |
| 198 | WLQQRPGQSPRRLIS | 37 |
| 199 | GVPDRFSGSGSGTDFTLKISRVEAEDVGVYYC | 38 |
| 200 | FGQGTKLEIK | 39 |
| 201 | QGQDRHMIRMRQLIDIVDQLKNYVNDLVPEFLPAPEDVETNCEWSAFSCFQK | IL-21 (156) |
| | AQLKSANTGNNERIINVSIKKLKRKPPSTNAGRRQKHRLTCPSCDSYEKKPPKE | |
| | FLERFKSLLQKMIHQHLSSRTHGSEDS | |
| 202 | QGQDEHMIRMRQLIDIVDQLKNYVNDLVPEFLPAPEDVETNCEWSAFSCFQK | IL-21 (157) |
| | AQLKSANTGNNERIINVSIKKLKRKPPSTNAGRRQKHRLTCPSCDSYEKKPPKE | |
| | FLERFKSLLQKMIHQHLSSRTHGSEDS | |
| 203 | QGQDRHMIRMRQLIDIVDQLKNYVNDLVPEFLPAPEDVETNCEWSAFSCFQK | IL-21 (158) |
| | AQLKSANTGNNERIINVSIKKLKRKPPSTNAGRRQKHRLTCPSCDSYEKKPPKE | |
| | FLERFKSLLQKMIHQHL | |
| 204 | MDCDIEGKDGKQYESVLMVSIDQLLDSMKEIGSNCLNNEFNFFKRHICDANK | IL-7 (1) |
| | EGMFLFRAARKLRQFLKMNSTGDFDLHLLKVSEGTTILLNCTGQVKGRKPAA | |
| | LGEAQPTKSLEENKSLKEQKKLNDLCFLKRLLQEIKTCWNKILMGTKEH | |
| 205 | DCDIEGKDGKQYESVLMVSIDQLLDSMKEIGSNCLNNEFNFFKRHICDANKEG | IL-7 (2) |
| | MFLFRAARKLRQFLKMNSTGDFDLHLLKVSEGTTILLNCTGQVKGRKPAALG | |
| | EAQPTKSLEENKSLKEQKKLNDLCFLKRLLQEIKTCWNKILMGTKEH | |
| 206 | DCDIEGKDGKQYESVLMVSIDQLLDSMKEIGSNCLNNEFNFFKRHICDANKEG | IL-7 (3) |
| | MFLFRAARKLRQFLKMNSTGDFDLHLLKVSEGTTILLNCTGQEENKSLKEQK | |
| | KLNDLCFLKRLLQEIKTCWNKILMGTKEH | |
| 207 | DCDIEGKDGKQYESVLMVSIDQLLDSMKEIGSNCLNNEFNFFKRHICDANKEG | IL-7 (4) |
| | MFLFRAARKLRQFLKMNSTGDFDLHLLKVSEGTTILLKEQKKLNDLCFLKRL | |
| | LQEIKTCWNKILMGTKEH | |
| 208 | DCDIEGKDGKQYESVLMVSIDQLLDSMKEIGSNCLNNEFNFFKRHICDANKV | IL-7 (5) |
| | KGRKPAALGEAQPTKSLEENESLKEQKKLNDLCFLKRLLQEIKTCWNKILMGT | |
| | KEH | |
| 209 | DCDIEGKDGKQYESVLMVSIDQLLDSMKEIGSNCLNNEFNFFKRHICDANKEE | IL-7 (6) |
| | NESLKEQKKLNDLCFLKRLLQEIKTCWNKILMGTKEH | |
| 210 | DCDIEGKDGKQYESVLMVSIDQLLVKGRKPAALGEAQPTKSLEENESLKEQK | IL-7 (7) |
| | KLNDLCFLKRLLQEIKTCWNKILMGTKEH | |
| 211 | DCDIEGKDGKQYESVLMVSIDQLLEENKSLKEQKKLNDLCFLKRLLQEIKTC | IL-7 (8) |
| | WNKILMGTKEH | |
| 212 | DCDIEGKDGKQYESVLMVSIDQLLDSMKEIGSNCL | IL-7 (mature |
| | NNEFNFFKRHICDANKVKGRKPAALGEAQPTKSLEENKSLKEQKKLNDLCFL | human IL-7) |
| | KRLLQEIK | |
| | TCWNKILMGTKEH | |
| 213 | HKSSSQGQDRHMIRMRQLIDIVDQLKNYVNDLVPEF | IL-21 (mature |
| | LPAPEDVETNCEWSAFSCFQKAQLKSANTGNNERIINVSIKKLKRKPPSTNAGR | human IL-21) |
| | RQKHRL | |
| | TCPSCDSYEKKPPKEFLERFKSLLQKMIHQHLSSRTHGSEDS | |
| 214 | QVQLVQSGAEVKKPGASVKVSCKASGGTFSSYAISWVRQAPGQGLEWMGIID | CD8_1 VH |
| | PSDGNTNYAQNFQGRVTMTRDTSTSTVYMELSSLRSEDTAVYYCAKERAAAG | |
| | YYYYMDVWGQGTTVTVSS | |
| 215 | DIQMTQSPSSLSASVGDRVTITCRASQSISSYLNWYQQKPGKAPKLLIYAASSL | CD8_1 VL |
| | QSGVPSRFSGSGSGTDFTLTISSLQPEDFATYYCQQSYSTPLTFGGGTKVEIKR | |
| 216 | QVQLVQSGAEVKKPGASVKVSCKASGYTFTDYYIQWVRQAPGQGLEWMGW | CD8_2 VH |
| | INPNSGGTSYAQKFQGRVTMTRDTSTSTVYMELSSLRSEDTAVYYCAKEGDY | |
| | YYGMDAWGQGTMVTVSS | |
| 217 | DIVMTQSPLSLPVTPGEPASISCRSSQSLLHSNGYNYLDWYLQKPGQSPQLLIY | CD8_2 VL |
| | LGSNRASGVPDRFSGSGSGTDFTLKISRVEAEDVGVYYCMQGLQTPHTFGQG | |
| | TKVEIKR | |
| 218 | QVQLVQSGAEVKKPGASVKVSCKASGYTFTSYYMHWVRQAPGQGLEWMGG | CD8_3 VH |
| | FDPEDGETIYAQKFQGRVTMTRDTSTSTVYMELSSLRSEDTAVYYCARDQGW | |
| | GMDVWGQGTTVTVSS | |
| 219 | DIQMTQSPSSLSASVGDRVTITCRASQSISSYLNWYQQKPGKAPKLLIYAASSL | CD8_3 VL |
| | QSGVPSRFSGSGSGTDFTLTISSLQPEDFATYYCQQTYSTPYTFGQGTKLEIKR | |
| 220 | QVQLVQSGAEVKKPGASVKVSCKASGYTFTNHYMHWVRQAPGQGLEWMG | CD8_4 VH |
| | WMNPNSGNTGYAQKFQGRVTMTRDTSTSTVYMELSSLRSEDTAVYYCASSES | |
| | GSDLDYWGQGTLVTVSS | |
| 221 | DIQMTQSPSSLSASVGDRVTITCRASQTIGNYVNWYQQKPGKAPKLLIYGASN | CD8_4 VL |
| | LHTGVPSRFSGSGSGTDFTLTISSLQPEDFATYYCQQTYSAPLTFGGGTKVEIKR | |
| 222 | QVQLVESGGGLVQAGGSLRLSCAASGRTFSGYVMGWFRQAPGKQRKFVAAIS | CD8_VHH |
| | RGGLSTSYADSVKGRFTISRDNAKNTVFLQMNTLKPEDTAVYYCAADRSDLY | |
| | EITAASNIDSWGQGTLVTVSS | |
| 223 | SYAIS | CDR-H1 |
| 224 | IIDPSDGNTNYAQNFQG | CDR-H2 |
| 225 | ERAAAGYYYYMDV | CDR-H3 |
| 226 | RASQSISSYLN | CDR-L1 |
| 227 | AASSLqS | CDR-L2 |
| 228 | QQSYSTPLT | CDR-L3 |
| 229 | QVQLVQSGAEVKKPGASVKVSCKASGGTFSSYAISWVRQAPGQGLEWMGIID | CD8 scFv |
| | PSDGNTNYAQNFQGRVTMTRDTSTSTVYMELSSLRSEDTAVYYCAKERAAAG | |
| | YYYYMDVWGQGTTVTVSSGGGGSGGGGSGGGGSDIQMTQSPSSLSASVGDR | |
| | VTITCRASQSISSYLNWYQQKPGKAPKLLIYAASSLQSGVPSRFSGSGSGTDFT | |
| | LTISSLQPEDFATYYCQQSYSTPLTFGGGTKVEIKR | |
| 230 | DYYIQ | CDR-H1 |
| 231 | WINPNSGGTSYAQKFQG | CDR-H2 |
| 232 | EGDYYYGMDA | CDR-H3 |
| 233 | RSSQSLLHSNGYNYLD | CDR-L1 |
| 234 | LGSNRAS | CDR-L2 |
| 235 | MQGLQTPHT | CDR-L3 |
| 236 | QVQLVQSGAEVKKPGASVKVSCKASGYTFTDYYIQWVRQAPGQGLEWMGW | CD8 scFv |
| | INPNSGGTSYAQKFQGRVTMTRDTSTSTVYMELSSLRSEDTAVYYCAKEGDY | |
| | YYGMDAWGQGTMVTVSSGGGGSGGGGSGGGGSDIVMTQSPLSLPVTPGEPA | |
| | SISCRSSQSLLHSNGYNYLDWYLQKPGQSPQLLIYLGSNRASGVPDRFSGSGS | |
| | GTDFTLKISRVEAEDVGVYYCMQGLQTPHTFGQGTKVEIKR | |
| 237 | SYYMH | CDR-H1 |
| 238 | GFDPEDGETIYAQKFQG | CDR-H2 |
| 239 | DQGWGMDV | CDR-H3 |
| 240 | QQTYSTPYT | CDR-L3 |
| 241 | QVQLVQSGAEVKKPGASVKVSCKASGYTFTSYYMHWVRQAPGQGLEWMGG | CD8 scFv |
| | FDPEDGETIYAQKFQGRVTMTRDTSTSTVYMELSSLRSEDTAVYYCARDQGW | |
| | GMDVWGQGTTVTVSSGGGGSGGGGSGGGGSDIQMTQSPSSLSASVGDRVTIT | |
| | CRASQSISSYLNWYQQKPGKAPKLLIYAASSLQSGVPSRFSGSGSGTDFTLTISS | |
| | LQPEDFATYYCQQTYSTPYTFGQGTKLEIKR | |
| 242 | NHYMH | CDR-H1 |
| 243 | WMNPNSGNTGYAQKFQG | CDR-H2 |
| 244 | SESGSDLDY | CDR-H3 |
| 245 | RASQTIGNYVN | CDR-L1 |
| 246 | GASNLHT | CDR-L2 |
| 247 | QQTYSAPLT | CDR-L3 |
| 248 | QVQLVQSGAEVKKPGASVKVSCKASGYTFTNHYMHWVRQAPGQGLEWMG | CD8 scFv |
| | WMNPNSGNTGYAQKFQGRVTMTRDTSTSTVYMELSSLRSEDTAVYYCASSES | |
| | GSDLDYWGQGTLVTVSSGGGGSGGGGSGGGGSDIQMTQSPSSLSASVGDRVT | |
| | ITCRASQTIGNYVNWYQQKPGKAPKLLIYGASNLHTGVPSRFSGSGSGTDFTL | |
| | TISSLQPEDFATYYCQQTYSAPLTFGGGTKVEIKR | |
| 249 | GYVMG | CDR-H1 |
| 250 | AISRGGLSTSYADSVKG | CDR-H2 |
| 251 | DRSDLYEITAASNIDS | CDR-H3 |
| 252 | GFDDPRKAIELVQKRYGRPCDCSGGQVSEPPSDRVSQVTCSGKTAYLMPDQR | Full BaEV |
| | WKCKSIPKDTSPSGPLQECPCNSYQSSVHSSCYTSYQQCRSGNKTYYTATLLK | protein without |
| | TQTGGTSDVQVLGSTNKLIQSPCNGIKGQSICWSTTAPIHVSDGGGPLDTTRIK | signal sequence |
| | SVQRKLEEIHKALYPELQYHPLAIPKVRDNLMVDAQTLNILNATYNLLLMSNT | (cytoplasmic |
| | SLVDDCWLCLKLGPPTPLAIPNFLLSYVTRSSDNISCLIIPPLLVQPMQFSNSSCL | tail, bold and |
| | FSPSYNSTEEIDLGHVAFSNCTSITNVTGPICAVNGSVFLCGNNMAYTYLPTNW | underline) |
| | TGLCVLATLLPDIDIIPGDEPVPIPAIDHFIYRPKRAIQFIPLLAGLGITAAFTTGAT | |
| | GLGVSVTQYTKLSNQLISDVQILSSTIQDLQDQVDSLAEVVLQNRRGLDLLTA | |
| | EQGGICLALQEKCCFYVNKSGIVRDKIKTLQEELERRRKDLASNPLWTGLQGL | |
| | LPYLLPFLGPLLTLLLLLTIGPCIFNRLTAFINDKLNIIHAMVLTQQYQVLRTD | |
| | EEAqD | |
| 253 | VLTQQYQVLRTDEEAQD | Full R peptide |
| | | only |
| 254 | GFDDPRKAIELVQKRYGRPCDCSGGQVSEPPSDRVSQVTCSGKTAYLMPDQR | BaEv envelope |
| | WKCKSIPKDTSPSGPLQECPCNSYQSSVHSSCYTSYQQCRSGNKTYYTATLLK | glycoprotein |
| | TQTGGTSDVQVLGSTNKLIQSPCNGIKGQSICWSTTAPIHVSDGGGPLDTTRIK | R + 3 |
| | SVQRKLEEIHKALYPELQYHPLAIPKVRDNLMVDAQTLNILNATYNLLLMSNT | |
| | SLVDDCWLCLKLGPPTPLAIPNFLLSYVTRSSDNISCLIIPPLLVQPMQFSNSSCL | |
| | FSPSYNSTEEIDLGHVAFSNCTSITNVTGPICAVNGSVFLCGNNMAYTYLPTNW | |
| | TGLCVLATLLPDIDIIPGDEPVPIPAIDHFIYRPKRAIQFIPLLAGLGITAAFTTGAT | |
| | GLGVSVTQYTKLSNQLISDVQILSSTIQDLQDQVDSLAEVVLQNRRGLDLLTA | |
| | EQGGICLALQEKCCFYVNKSGIVRDKIKTLQEELERRRKDLASNPLWTGLQGL | |
| | LPYLLPFLGPLLTLLLLLTIGPCIFNRLTAFINDKLNIIHAMVLT | |
| 255 | GFDDPRKAIELVQKRYGRPCDCSGGQVSEPPSDRVSQVTCSGKTAYLMPDQR | BaEv envelope |
| | WKCKSIPKDTSPSGPLQECPCNSYQSSVHSSCYTSYQQCRSGNKTYYTATLLK | glycoprotein |
| | TQTGGTSDVQVLGSTNKLIQSPCNGIKGQSICWSTTAPIHVSDGGGPLDTTRIK | R + 4 |
| | SVQRKLEEIHKALYPELQYHPLAIPKVRDNLMVDAQTLNILNATYNLLLMSNT | |
| | SLVDDCWLCLKLGPPTPLAIPNFLLSYVTRSSDNISCLIIPPLLVQPMQFSNSSCL | |
| | FSPSYNSTEEIDLGHVAFSNCTSITNVTGPICAVNGSVFLCGNNMAYTYLPTNW | |
| | TGLCVLATLLPDIDIIPGDEPVPIPAIDHFIYRPKRAIQFIPLLAGLGITAAFTTGAT | |
| | GLGVSVTQYTKLSNQLISDVQILSSTIQDLQDQVDSLAEVVLQNRRGLDLLTA | |
| | EQGGICLALQEKCCFYVNKSGIVRDKIKTLQEELERRRKDLASNPLWTGLQGL | |
| | LPYLLPFLGPLLTLLLLLTIGPCIFNRLTAFINDKLNIIHAMVLTQ | |
| 256 | GFDDPRKAIELVQKRYGRPCDCSGGQVSEPPSDRVSQVTCSGKTAYLMPDQR | BaEv envelope |
| | WKCKSIPKDTSPSGPLQECPCNSYQSSVHSSCYTSYQQCRSGNKTYYTATLLK | glycoprotein |
| | TQTGGTSDVQVLGSTNKLIQSPCNGIKGQSICWSTTAPIHVSDGGGPLDTTRIK | R + 5 |
| | SVQRKLEEIHKALYPELQYHPLAIPKVRDNLMVDAQTLNILNATYNLLLMSNT | |
| | SLVDDCWLCLKLGPPTPLAIPNFLLSYVTRSSDNISCLIIPPLLVQPMQFSNSSCL | |
| | FSPSYNSTEEIDLGHVAFSNCTSITNVTGPICAVNGSVFLCGNNMAYTYLPTNW | |
| | TGLCVLATLLPDIDIIPGDEPVPIPAIDHFIYRPKRAIQFIPLLAGLGITAAFTTGAT | |
| | GLGVSVTQYTKLSNQLISDVQILSSTIQDLQDQVDSLAEVVLQNRRGLDLLTA | |
| | EQGGICLALQEKCCFYVNKSGIVRDKIKTLQEELERRRKDLASNPLWTGLQGL | |
| | LPYLLPFLGPLLTLLLLLTIGPCIFNRLTAFINDKLNIIHAMVLTQQ | |
| 257 | GFDDPRKAIELVQKRYGRPCDCSGGQVSEPPSDRVSQVTCSGKTAYLMPDQR | BaEv envelope |
| | WKCKSIPKDTSPSGPLQECPCNSYQSSVHSSCYTSYQQCRSGNKTYYTATLLK | glycoprotein |
| | TQTGGTSDVQVLGSTNKLIQSPCNGIKGQSICWSTTAPIHVSDGGGPLDTTRIK | R + 6 |
| | SVQRKLEEIHKALYPELQYHPLAIPKVRDNLMVDAQTLNILNATYNLLLMSNT | |
| | SLVDDCWLCLKLGPPTPLAIPNFLLSYVTRSSDNISCLIIPPLLVQPMQFSNSSCL | |
| | FSPSYNSTEEIDLGHVAFSNCTSITNVTGPICAVNGSVFLCGNNMAYTYLPTNW | |
| | TGLCVLATLLPDIDIIPGDEPVPIPAIDHFIYRPKRAIQFIPLLAGLGITAAFTTGAT | |
| | GLGVSVTQYTKLSNQLISDVQILSSTIQDLQDQVDSLAEVVLQNRRGLDLLTA | |
| | EQGGICLALQEKCCFYVNKSGIVRDKIKTLQEELERRRKDLASNPLWTGLQGL | |
| | LPYLLPFLGPLLTLLLLLTIGPCIFNRLTAFINDKLNIIHAMVLTQQY | |
| 258 | GFDDPRKAIELVQKRYGRPCDCSGGQVSEPPSDRVSQVTCSGKTAYLMPDQR | BaEv envelope |
| | WKCKSIPKDTSPSGPLQECPCNSYQSSVHSSCYTSYQQCRSGNKTYYTATLLK | glycoprotein |
| | TQTGGTSDVQVLGSTNKLIQSPCNGIKGQSICWSTTAPIHVSDGGGPLDTTRIK | R + 7 |
| | SVQRKLEEIHKALYPELQYHPLAIPKVRDNLMVDAQTLNILNATYNLLLMSNT | |
| | SLVDDCWLCLKLGPPTPLAIPNFLLSYVTRSSDNISCLIIPPLLVQPMQFSNSSCL | |
| | FSPSYNSTEEIDLGHVAFSNCTSITNVTGPICAVNGSVFLCGNNMAYTYLPTNW | |
| | TGLCVLATLLPDIDIIPGDEPVPIPAIDHFIYRPKRAIQFIPLLAGLGITAAFTTGAT | |
| | GLGVSVTQYTKLSNQLISDVQILSSTIQDLQDQVDSLAEVVLQNRRGLDLLTA | |
| | EQGGICLALQEKCCFYVNKSGIVRDKIKTLQEELERRRKDLASNPLWTGLQGL | |
| | LPYLLPFLGPLLTLLLLLTIGPCIFNRLTAFINDKLNIIHAMVLTQQYQ | |
| 259 | GFDDPRKAIELVQKRYGRPCDCSGGQVSEPPSDRVSQVTCSGKTAYLMPDQR | BaEv envelope |
| | WKCKSIPKDTSPSGPLQECPCNSYQSSVHSSCYTSYQQCRSGNKTYYTATLLK | glycoprotein |
| | TQTGGTSDVQVLGSTNKLIQSPCNGIKGQSICWSTTAPIHVSDGGGPLDTTRIK | R + 8 |
| | SVQRKLEEIHKALYPELQYHPLAIPKVRDNLMVDAQTLNILNATYNLLLMSNT | |
| | SLVDDCWLCLKLGPPTPLAIPNFLLSYVTRSSDNISCLIIPPLLVQPMQFSNSSCL | |
| | FSPSYNSTEEIDLGHVAFSNCTSITNVTGPICAVNGSVFLCGNNMAYTYLPTNW | |
| | TGLCVLATLLPDIDIIPGDEPVPIPAIDHFIYRPKRAIQFIPLLAGLGITAAFTTGAT | |
| | GLGVSVTQYTKLSNQLISDVQILSSTIQDLQDQVDSLAEVVLQNRRGLDLLTA | |
| | EQGGICLALQEKCCFYVNKSGIVRDKIKTLQEELERRRKDLASNPLWTGLQGL | |
| | LPYLLPFLGPLLTLLLLLTIGPCIFNRLTAFINDKLNIIHAMVLTQQYQV | |
| 260 | GFDDPRKAIELVQKRYGRPCDCSGGQVSEPPSDRVSQVTCSGKTAYLMPDQR | BaEv envelope |
| | WKCKSIPKDTSPSGPLQECPCNSYQSSVHSSCYTSYQQCRSGNKTYYTATLLK | glycoprotein |
| | TQTGGTSDVQVLGSTNKLIQSPCNGIKGQSICWSTTAPIHVSDGGGPLDTTRIK | R + 9 |
| | SVQRKLEEIHKALYPELQYHPLAIPKVRDNLMVDAQTLNILNATYNLLLMSNT | |
| | SLVDDCWLCLKLGPPTPLAIPNFLLSYVTRSSDNISCLIIPPLLVQPMQFSNSSCL | |
| | FSPSYNSTEEIDLGHVAFSNCTSITNVTGPICAVNGSVFLCGNNMAYTYLPTNW | |
| | TGLCVLATLLPDIDIIPGDEPVPIPAIDHFIYRPKRAIQFIPLLAGLGITAAFTTGAT | |
| | GLGVSVTQYTKLSNQLISDVQILSSTIQDLQDQVDSLAEVVLQNRRGLDLLTA | |
| | EQGGICLALQEKCCFYVNKSGIVRDKIKTLQEELERRRKDLASNPLWTGLQGL | |
| | LPYLLPFLGPLLTLLLLLTIGPCIFNRLTAFINDKLNIIHAMVLTQQYQVL | |
| 261 | MGFTTKIIFLYNLVLVYAGFDDPRKAIELVQKRYGRPCDCSGGQVSEPPSDRVS | BaEVRLess |
| | QVTCSGKTAYLMPDQRWKCKSIPKDTSPSGPLQECPCNSYQSSVHSSCYTSYQ | |
| | QCRSGNKTYYTATLLKTQTGGTSDVQVLGSTNKLIQSPCNGIKGQSICWSTTA | |
| | PIHVSDGGGPLDTTRIKSVQRKLEEIHKALYPELQYHPLAIPKVRDNLMVDAQ | |
| | TLNILNATYNLLLMSNTSLVDDCWLCLKLGPPTPLAIPNFLLSYVTRSSDNISCL | |
| | IIPPLLVQPMQFSNSSCLFSPSYNSTEEIDLGHVAFSNCTSITNVTGPICAVNGSV | |
| | FLCGNNMAYTYLPTNWTGLCVLATLLPDIDIIPGDEPVPIPAIDHFIYRPKRAIQF | |
| | IPLLAGLGITAAFTTGATGLGVSVTQYTKLSNQLISDVQILSSTIQDLQDQVDSL | |
| | AEVVLQNRRGLDLLTAEQGGICLALQEKCCFYVNKSGIVRDKIKTLQEELERR | |
| | RKDLASNPLWTGLQGLLPYLLPFLGPLLTLLLLLTIGPCIFNRLTAFINDKLNIIH | |
| | AM | |
| 262 | MGFTTKIIFLYNLVLVYAGFDDPRKAIELVQKRYGRPCDCSGGQVSEPPSDRVS | BaEVTR |
| | QVTCSGKTAYLMPDQRWKCKSIPKDTSPSGPLQECPCNSYQSSVHSSCYTSYQ | |
| | QCRSGNKTYYTATLLKTQTGGTSDVQVLGSTNKLIQSPCNGIKGQSICWSTTA | |
| | PIHVSDGGGPLDTTRIKSVQRKLEEIHKALYPELQYHPLAIPKVRDNLMVDAQ | |
| | TLNILNATYNLLLMSNTSLVDDCWLCLKLGPPTPLAIPNFLLSYVTRSSDNISCL | |
| | IIPPLLVQPMQFSNSSCLFSPSYNSTEEIDLGHVAFSNCTSITNVTGPICAVNGSV | |
| | FLCGNNMAYTYLPTNWTGLCVLATLLPDIDIIPGDEPVPIPAIDHFIYRPKRAIQF | |
| | IPLLAGLGITAAFTTGATGLGVSVTQYTKLSNQLISDVQILSSTIQDLQDQVDSL | |
| | AEVVLQNRRGLDLLTAEQGGICLALQEKCCFYVNKSGIVRDKIKTLQEELERR | |
| | RKDLASNPLWTGLQGLLPYLLPFLGPLLTLLLLLTIGPCIFNRLVQFVKDRISVV | |
| | QALVLTQQYHQLKPLEYEP | |
| 263 | GGGGS | Linker |
| 264 | GGGGGS | Linker |
| 265 | (GGGGS)n | Linker |
| | | n is 1 to 10 |
| 266 | (GGGGGS)n | Linker |
| | | n is 1 to 6 |
| 267 | (GmS)n | Linker |
| | | Each of m and n |
| | | is an integer |
| | | between 1 to 4, |
| | | inclusive |
| 268 | (GGS)n | Linker |
| | | n is 1 to 10 |
| 269- | | Reserved |
| 374 | | |
| 375 | MSPQRDRINAFYKDNPHPKGSRIVINREHLMIDRPYVLLAVLFVMFLSLIGLLA | Measles H |
| | IAGIRLHRAAIYTAEIHKSLSTNLDVTNSIEHQVKDVLTPLFKIIGDEVGLRTPQR | protein |
| | FTDLVKFISDKIKFLNPDREYDFRDLTWCINPPERIKLDYDQYCADVAAEELMN | |
| | ALVNSTLLETRTTNQFLAVSKGNCSGPTTIRGQFSNMSLSLLDLYLGRGYNVSS | |
| | IVTMTSQGMYGGTYLVEKPNLSSKRSELSQLSMYRVFEVGVIRNPGLGAPVFH | |
| | MTNYLEQPVSNDLSNCMVALGELKLAALCHGEDSITIPYQGSGKGVSFQLVKL | |
| | GVWKSPTDMQSWVPLSTDD- | |
| | PVIDRLYLSSHRGVIADNQAKWAVPTTRTDDKLRMETCFQQACKGKIQALCE | |
| | NPEWAPLKDNRIPSYGVLSVDLSLTVELKIKIASGFGPLITHGSGMDLYKSNHN | |
| | NVYWLTIPPMKNLALGVINTLEWIPRFKVSPYLFNVPIKEAGEDCHAPTYLPAE | |
| | VDGDVKLSSNLVILPGQDLQYVLATYDTSRVEHAVVYYVYSPSRSFSYFYPFR | |
| | LPIKGVPIELQVECFTWDQKLWCRHFCVLADSESGGHITHSGMEGMGVSCTV | |
| | TREDGTNRR | |
| 376 | MLPYQDKVGAFYKDNARANSTKLSLVTEGHGGRRPPYLLFVLLILLVGILALL | Canine |
| | AITGVRFHQVSTSNMEFSRLLKEDMEKSEAVHHQVIDVLTPLFKIIGDEIGLRLP | distemper virus |
| | QKLNEIKQFILQKTNFFNPNREFDFRDLHWCINPPSTVKVNFTNYCESIGIRKAI | H protein |
| | ASAANPILLSALSGGRGDIFPPHRCSGATTSVGKVFPLSVSLSMSLISRTSEVIN | |
| | MLTAISDGVYGKTYLLVPDDIEREFDTREIRVFEIGFIKRWLNDMPLLQTTNYM | |
| | VLPKNSKAKVCTIAVGELTLASLCVEESTVLLYHDSSGSQDGILVVTLGIFWATP | |
| | MDHIEEVIPVAHPSMKKIHITNHRGFIKDSIATWMVPALASEKQEEQKGCLESA | |
| | CQRKTYPMCNQASWEPFGGRQLPSYGRLTLPLDASVDLQLNISFTYGPVILNG | |
| | DGMDYYESPLLNSGWLTIPPKDGTISGLINKAGRGDQFTVLPHVLTFAPRESSG | |
| | NCYLPIQTSQIRDRDVLIESNIVVLPTQSIRYVIATYDISRSDHAIVYYVYDPIRTI | |
| | SYTHPFRLTTKGRPDFLRIECFVWDDNLWCHQF | |
| 377 | MKKINEGLLDSKILSAFNTVIALLGSIVIIVMNIMIIQQYTRSTDNQAVIKDALQ | NivG.690 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRNNTVISRPGQSQCPRFNTCPAICAEGVYNDAFLIDRINWI | |
| | SAGVFLDSNATAANPVFTVFKDNEILYRAQLASEDTNAQKTITNCFLLKNKIW | |
| | CISLVEIYDTGDNVIRPKLFAVKIPEQCT | |
| 378 | MKKINEGLLDSKILSAFNTVIALLGSIVIIVMNIMIIQNYTRSTDNQAVIKDALQ | NivG.691 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRNNTVISRPGQSQCPRENTCPAICAEGVYNDAFLIDRINWI | |
| | SAGVELDSNATAANPVFTVFKDNEILYRAQLASEDTNAQKTITNCFLLKNKIW | |
| | CISLVEIYDTGDNVIRPKLFAVKIPEQCT | |
| 379 | MKKINEGLLDSKILSAFNTVIALLGSIVIIVMNIMIIQNYTRSTDNQAVIKDALQ | NivG.693 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRNNTVISRPGQSQCPRFNTCPAICAEGVYNDAFLIDRINWI | |
| | SAGVFLDSNATAANPVFTVFKDNEILYRAQLASEDTNAQKTITNCFLLKNKIW | |
| | CISLVEIYDTGDNVIRPKLFAVKIPEQCT | |
| 380 | MKKINEGLLDSKILSAFNTVIALLGSIVIIVMNIMIIQNYTRSTDNQAVIKDALQ | NivG.694 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRNNTVISRPGQSQCPRFNTCPAICAEGVYNDAFLIDRINWI | |
| | SAGVFLDSNATAANPVFTVFKDNEILYRAQLASEDTNAQKTITNCFLLKNKIW | |
| | CISLVEIYDTGDNVIRPKLFAVKIPEQCT | |
| 381 | MKKINEGLLDSKILSAFNTVIALLGSIVIIVMNIMIIQNYTRSTDNQAVIKDALQ | NivG.695 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRNNTVISRPGQSQCPRFNTCPAICAEGVYNDAFLIDRINWI | |
| | SAGVELDSNATAANPVFTVFKDNEILYRAQLASEDTNAQKTITNCFLLKNKIW | |
| | CISLVEIYDTGDNVIRPKLFAVKIPEQCT | |
| 382 | MKKINEGLLDSKILSAFNTVIALLGSIVIIVMNIMIIQNYTRSTDNQAVIKDALQ | NivG.696 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRQNTVISRPGQSQCPRENTCPAICAEGVYNDAFLIDRINWI | |
| | SAGVFLDSNATAANPVFTVFKDNEILYRAQLASEDTNAQKTITNCFLLKNKIW | |
| | CISLVEIYDTGDNVIRPKLFAVKIPEQCT | |
| 383 | MKKINEGLLDSKILSAFNTVIALLGSIVIIVMNIMIIQNYTRSTDNQAVIKDALQ | NivG.697 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRNNTVISRPGQSQCPRFNTCPAICAEGVYNDAFLIDRINWI | |
| | SAGVFLDSQATAANPVFTVFKDNEILYRAQLASEDTNAQKTITNCFLLKNKIW | |
| | CISLVEIYDTGDNVIRPKLFAVKIPEQCT | |
| 384 | MKKINEGLLDSKILSAFNTVIALLGSIVIIVMNIMIIQNYTRSTDNQAVIKDALQ | NivG.699 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRQNTVISRPGQSQCPRFNTCPAICAEGVYNDAFLIDRINWI | |
| | SAGVFLDSNATAANPVFTVFKDNEILYRAQLASEDTNAQKTITNCFLLKNKIW | |
| | CISLVEIYDTGDNVIRPKLFAVKIPEQCT | |
| 385 | MKKINEGLLDSKILSAFNTVIALLGSIVIIVMNIMIIQNYTRSTDNQAVIKDALQ | NivG.700 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRQNTVISRPGQSQCPRFNTCPAICAEGVYNDAFLIDRINWI | |
| | SAGVELDSNATAANPVFTVFKDNEILYRAQLASEDTNAQKTITNCFLLKNKIW | |
| | CISLVEIYDTGDNVIRPKLFAVKIPEQCT | |
| 386 | MKKINEGLLDSKILSAFNTVIALLGSIVIIVMNIMIIQNYTRSTDNQAVIKDALQ | NivG.701 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRNNTVISRPGQSQCPRFNTCPAICAEGVYNDAFLIDRINWI | |
| | SAGVFLDSQATAANPVFTVFKDNEILYRAQLASEDTNAQKTITNCFLLKNKIW | |
| | CISLVEIYDTGDNVIRPKLFAVKIPEQCT | |
| 387 | MKKINEGLLDSKILSAFNTVIALLGSIVIIVMNIMIIQQYTRSTDNQAVIKDALQ | NivG.702 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRQNTVISRPGQSQCPRFNTCPAICAEGVYNDAFLIDRINWI | |
| | SAGVFLDSQATAANPVFTVFKDNEILYRAQLASEDTNAQKTITNCFLLKNKIW | |
| | CISLVEIYDTGDNVIRPKLFAVKIPEQCT | |
| 388 | MKKINEGLLDSKILSAFNTVIALLGSIVIIVMNIMIIQNYTRSTDNQAVIKDALQ | NivG.740 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRQNTVISRPGQSQCPRFNTCPAICAEGVYNDAFLIDRINWI | |
| | SAGVFLDSQATAANPVFTVFKDNEILYRAQLASEDTNAQKTITNCFLLKNKIW | |
| | CISLVEIYDTGDNVIRPKLFAVKIPEQCT | |
| 389 | MKKINEGLLDSKILSAFNTVIALLGSIVIIVMNIMIIQNYTRSTDNQAVIKDALQ | NivG.742 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRNNTVISRPGQSQCPRFNTCPAICAEGVYNDAFLIDRINWI | |
| | SAGVFLDSQATAANPVFTVFKDNEILYRAQLASEDTNAQKTITNCFLLKNKIW | |
| | CISLVEIYDTGDNVIRPKLFAVKIPEQCT | |
| 390 | MKKINEGLLDSKILSAFNTVIALLGSIVIIVMNIMIIQNYTRSTDNQAVIKDALQ | NivG.743 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRQNTVISRPGQSQCPRFNTCPAICAEGVYNDAFLIDRINWI | |
| | SAGVELDSNATAANPVFTVFKDNEILYRAQLASEDTNAQKTITNCFLLKNKIW | |
| | CISLVEIYDTGDNVIRPKLFAVKIPEQCT | |
| 391 | MKKINEGLLDSKILSAFNTVIALLGSIVIIVMNIMIIQNYTRSTDNQAVIKDALQ | NivG.744 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRQNTVISRPGQSQCPRENTCPAICAEGVYNDAFLIDRINWI | |
| | SAGVFLDSQATAANPVFTVFKDNEILYRAQLASEDTNAQKTITNCFLLKNKIW | |
| | CISLVEIYDTGDNVIRPKLFAVKIPEQCT | |
| 392 | MKKINEGLLDSKILSAFNTVIALLGSIVIIVMNIMIIQNYTRSTDNQAVIKDALQ | NivG.748 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRQNTVISRPGQSQCPRFNTCPAICAEGVYNDAFLIDRINWI | |
| | SAGVFLDSQATAANPVFTVFKDNEILYRAQLASEDTNAQKTITNCFLLKNKIW | |
| | CISLVEIYDTGDNVIRPKLFAVKIPEQCT | |
| 393 | MKKINEGLLDSKILSAFNTVIALLGSIVIIVMNIMIIQNYTRSTDNQAVIKDALQ | NivG.749 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRNNTVISRPGQSQCPRFNTCPAICAEGVYNDAFLIDRINWI | |
| | SAGVFLDSNATAANPVFTVFKDNEILYRAQLASEDTNAQKTITNCFLLKNKIW | |
| | CISLVEIYDTGDNVIRPKLFAVKIPEQCT | |
| 394 | MKKINEGLLDSKILSAFNTVIALLGSIVIIVMNIMIIQNYTRSTDNQAVIKDALQ | NivG.750 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRNNTVISRPGQSQCPRFNTCPAICAEGVYNDAFLIDRINWI | |
| | SAGVELDSQATAANPVFTVFKDNEILYRAQLASEDTNAQKTITNCFLLKNKIW | |
| | CISLVEIYDTGDNVIRPKLFAVKIPEQCT | |
| 395 | MKKINEGLLDSKILSAFNTVIALLGSIVIIVMNIMIIQNYTRSTDNQAVIKDALQ | NivG.752 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRQNTVISRPGQSQCPRFNTCPAICAEGVYNDAFLIDRINWI | |
| | SAGVFLDSQATAANPVFTVFKDNEILYRAQLASEDTNAQKTITNCFLLKNKIW | |
| | CISLVEIYDTGDNVIRPKLFAVKIPEQCT | |
| 396 | MKKINEGLLDSKILSAFNTVIALLGSIVIIVMNIMIIQNYTRSTDNQAVIKDALQ | NivG.753 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRNNTVISRPGQSQCPRENTCPAICAEGVYNDAFLIDRINWI | |
| | SAGVFLDSQATAANPVFTVFKDNEILYRAQLASEDTNAQKTITNCFLLKNKIW | |
| | CISLVEIYDTGDNVIRPKLFAVKIPEQCT | |
| 397 | MKKINEGLLDSKILSAFNTVIALLGSIVIIVMNIMIIQNYTRSTDNQAVIKDALQ | NivG.754 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRQNTVISRPGQSQCPRFNTCPAICAEGVYNDAFLIDRINWI | |
| | SAGVFLDSNATAANPVFTVFKDNEILYRAQLASEDTNAQKTITNCFLLKNKIW | |
| | CISLVEIYDTGDNVIRPKLFAVKIPEQCT | |
| 398 | MKKINEGLLDSKILSAFNTVIALLGSIVIIVMNIMIIQNYTRSTDNQAVIKDALQ | NivG.755 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRQNTVISRPGQSQCPRFNTCPAICAEGVYNDAFLIDRINWI | |
| | SAGVFLDSQATAANPVFTVFKDNEILYRAQLASEDTNAQKTITNCFLLKNKIW | |
| | CISLVEIYDTGDNVIRPKLFAVKIPEQCT | |
| 399 | MKKINEGLLDSKILSAFNTVIALLGSIVIIVMNIMIIQNYTRSTDNQAVIKDALQ | NivG.756 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRNNTVISRPGQSQCPRFNTCPAICAEGVYNDAFLIDRINWI | |
| | SAGVELDSNATAANPVFTVFKDNEILYRAQLASEDTNAQKTITNCFLLKNKIW | |
| | CISLVEIYDTGDNVIRPKLFAVKIPEQCT | |
| 400 | MKKINEGLLDSKILSAFNTVIALLGSIVIIVMNIMIIQNYTRSTDNQAVIKDALQ | NivG.757 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRNNTVISRPGQSQCPRENTCPAICAEGVYNDAFLIDRINWI | |
| | SAGVFLDSQATAANPVFTVFKDNEILYRAQLASEDTNAQKTITNCFLLKNKIW | |
| | CISLVEIYDTGDNVIRPKLFAVKIPEQCT | |
| 401 | MKKINEGLLDSKILSAFNTVIALLGSIVIIVMNIMIIQNYTRSTDNQAVIKDALQ | NivG.758 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRQNTVISRPGQSQCPRFNTCPAICAEGVYNDAFLIDRINWI | |
| | SAGVELDSNATAANPVFTVFKDNEILYRAQLASEDTNAQKTITNCFLLKNKIW | |
| | CISLVEIYDTGDNVIRPKLFAVKIPEQCT | |
| 402 | MKKINEGLLDSKILSAFNTVIALLGSIVIIVMNIMIIQNYTRSTDNQAVIKDALQ | NivG.759 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRQNTVISRPGQSQCPRFNTCPAICAEGVYNDAFLIDRINWI | |
| | SAGVFLDSQATAANPVFTVFKDNEILYRAQLASEDTNAQKTITNCFLLKNKIW | |
| | CISLVEIYDTGDNVIRPKLFAVKIPEQCT | |
| 403 | MKKINEGLLDSKILSAFNTVIALLGSIVIIVMNIMIIQNYTRSTDNQAVIKDALQ | NivG.760 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRNNTVISRPGQSQCPRFNTCPAICAEGVYNDAFLIDRINWI | |
| | SAGVELDSNATAANPVFTVFKDNEILYRAQLASEDTNAQKTITNCFLLKNKIW | |
| | CISLVEIYDTGDNVIRPKLFAVKIPEQCT | |
| 404 | MKKINEGLLDSKILSAFNTVIALLGSIVIIVMNIMIIQNYTRSTDNQAVIKDALQ | NivG.761 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRNNTVISRPGQSQCPRFNTCPAICAEGVYNDAFLIDRINWI | |
| | SAGVFLDSQATAANPVFTVFKDNEILYRAQLASEDTNAQKTITNCFLLKNKIW | |
| | CISLVEIYDTGDNVIRPKLFAVKIPEQCT | |
| 405 | MKKINEGLLDSKILSAFNTVIALLGSIVIIVMNIMIIQNYTRSTDNQAVIKDALQ | NivG.762 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRQNTVISRPGQSQCPRENTCPAICAEGVYNDAFLIDRINWI | |
| | SAGVFLDSNATAANPVFTVFKDNEILYRAQLASEDTNAQKTITNCFLLKNKIW | |
| | CISLVEIYDTGDNVIRPKLFAVKIPEQCT | |
| 406 | MKKINEGLLDSKILSAFNTVIALLGSIVIIVMNIMIIQNYTRSTDNQAVIKDALQ | NivG.763 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRQNTVISRPGQSQCPRFNTCPAICAEGVYNDAFLIDRINWI | |
| | SAGVFLDSQATAANPVFTVFKDNEILYRAQLASEDTNAQKTITNCFLLKNKIW | |
| | CISLVEIYDTGDNVIRPKLFAVKIPEQCT | |
| 407 | MKKINEGLLDSKILSAFNTVIALLGSIVIIVMNIMIIQNYTRSTDNQAVIKDALQ | NivG.764 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRNNTVISRPGQSQCPRFNTCPAICAEGVYNDAFLIDRINWI | |
| | SAGVFLDSNATAANPVFTVFKDNEILYRAQLASEDTNAQKTITNCFLLKNKIW | |
| | CISLVEIYDTGDNVIRPKLFAVKIPEQCT | |
| 408 | MKKINEGLLDSKILSAFNTVIALLGSIVIIVMNIMIIQNYTRSTDNQAVIKDALQ | NivG.765 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRNNTVISRPGQSQCPRFNTCPAICAEGVYNDAFLIDRINWI | |
| | SAGVELDSQATAANPVFTVFKDNEILYRAQLASEDTNAQKTITNCFLLKNKIW | |
| | CISLVEIYDTGDNVIRPKLFAVKIPEQCT | |
| 409 | MKKINEGLLDSKILSAFNTVIALLGSIVIIVMNIMIIQNYTRSTDNQAVIKDALQ | NivG.766 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRQNTVISRPGQSQCPRENTCPAICAEGVYNDAFLIDRINWI | |
| | SAGVELDSNATAANPVFTVFKDNEILYRAQLASEDTNAQKTITNCFLLKNKIW | |
| | CISLVEIYDTGDNVIRPKLFAVKIPEQCT | |
| 410 | MKKINEGLLDSKILSAFNTVIALLGSIVIIVMNIMIIQNYTRSTDNQAVIKDALQ | NivG.767 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRQNTVISRPGQSQCPRFNTCPAICAEGVYNDAFLIDRINWI | |
| | SAGVFLDSQATAANPVFTVFKDNEILYRAQLASEDTNAQKTITNCFLLKNKIW | |
| | CISLVEIYDTGDNVIRPKLFAVKIPEQCT | |
| 411 | MKKINEGLLDSKILSAFNTVIALLGSIVIIVMNIMIIQNYTRSTDNQAVIKDALQ | NivG.768 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRNNTVISRPGQSQCPRFNTCPAICAEGVYNDAFLIDRINWI | |
| | SAGVFLDSQATAANPVFTVFKDNEILYRAQLASEDTNAQKTITNCFLLKNKIW | |
| | CISLVEIYDTGDNVIRPKLFAVKIPEQCT | |
| 412 | MKKINEGLLDSKILSAFNTVIALLGSIVIIVMNIMIIQNYTRSTDNQAVIKDALQ | NivG.769 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRQNTVISRPGQSQCPRFNTCPAICAEGVYNDAFLIDRINWI | |
| | SAGVELDSNATAANPVFTVFKDNEILYRAQLASEDTNAQKTITNCFLLKNKIW | |
| | CISLVEIYDTGDNVIRPKLFAVKIPEQCT | |
| 413 | MKKINEGLLDSKILSAFNTVIALLGSIVIIVMNIMIIQNYTRSTDNQAVIKDALQ | NivG.770 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRQNTVISRPGQSQCPRFNTCPAICAEGVYNDAFLIDRINWI | |
| | SAGVFLDSQATAANPVFTVFKDNEILYRAQLASEDTNAQKTITNCFLLKNKIW | |
| | CISLVEIYDTGDNVIRPKLFAVKIPEQCT | |
| 414 | MKKINEGLLDSKILSAFNTVIALLGSIVIIVMNIMIIQNYTRSTDNQAVIKDALQ | NivG.771 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRNNTVISRPGQSQCPRENTCPAICAEGVYNDAFLIDRINWI | |
| | SAGVFLDSNATAANPVFTVFKDNEILYRAQLASEDTNAQKTITNCFLLKNKIW | |
| | CISLVEIYDTGDNVIRPKLFAVKIPEQCT | |
| 415 | MKKINEGLLDSKILSAFNTVIALLGSIVIIVMNIMIIQNYTRSTDNQAVIKDALQ | NivG.772 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRNNTVISRPGQSQCPRFNTCPAICAEGVYNDAFLIDRINWI | |
| | SAGVFLDSQATAANPVFTVFKDNEILYRAQLASEDTNAQKTITNCFLLKNKIW | |
| | CISLVEIYDTGDNVIRPKLFAVKIPEQCT | |
| 416 | MKKINEGLLDSKILSAFNTVIALLGSIVIIVMNIMIIQNYTRSTDNQAVIKDALQ | NivG.773 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRQNTVISRPGQSQCPRFNTCPAICAEGVYNDAFLIDRINWI | |
| | SAGVFLDSNATAANPVFTVFKDNEILYRAQLASEDTNAQKTITNCFLLKNKIW | |
| | CISLVEIYDTGDNVIRPKLFAVKIPEQCT | |
| 417 | MKKINEGLLDSKILSAFNTVIALLGSIVIIVMNIMIIQNYTRSTDNQAVIKDALQ | NivG.774 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRQNTVISRPGQSQCPRFNTCPAICAEGVYNDAFLIDRINWI | |
| | SAGVELDSQATAANPVFTVFKDNEILYRAQLASEDTNAQKTITNCFLLKNKIW | |
| | CISLVEIYDTGDNVIRPKLFAVKIPEQCT | |
| 418 | MKKINEGLLDSKILSAFNTVIALLGSIVIIVMNIMIIQNYTRSTDNQAVIKDALQ | NivG.775 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRNNTVISRPGQSQCPRENTCPAICAEGVYNDAFLIDRINWI | |
| | SAGVFLDSNATAANPVFTVFKDNEILYRAQLASEDTNAQKTITNCFLLKNKIW | |
| | CISLVEIYDTGDNVIRPKLFAVKIPEQCT | |
| 419 | MKKINEGLLDSKILSAFNTVIALLGSIVIIVMNIMIIQNYTRSTDNQAVIKDALQ | NivG.776 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRNNTVISRPGQSQCPRFNTCPAICAEGVYNDAFLIDRINWI | |
| | SAGVFLDSQATAANPVFTVFKDNEILYRAQLASEDTNAQKTITNCFLLKNKIW | |
| | CISLVEIYDTGDNVIRPKLFAVKIPEQCT | |
| 420 | MKKINEGLLDSKILSAFNTVIALLGSIVIIVMNIMIIQNYTRSTDNQAVIKDALQ | NivG.777 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRQNTVISRPGQSQCPRFNTCPAICAEGVYNDAFLIDRINWI | |
| | SAGVFLDSNATAANPVFTVFKDNEILYRAQLASEDTNAQKTITNCFLLKNKIW | |
| | CISLVEIYDTGDNVIRPKLFAVKIPEQCT | |
| 421 | MKKINEGLLDSKILSAFNTVIALLGSIVIIVMNIMIIQNYTRSTDNQAVIKDALQ | NivG.778 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRQNTVISRPGQSQCPRFNTCPAICAEGVYNDAFLIDRINWI | |
| | SAGVELDSQATAANPVFTVFKDNEILYRAQLASEDTNAQKTITNCFLLKNKIW | |
| | CISLVEIYDTGDNVIRPKLFAVKIPEQCT | |
| 422 | MKKINEGLLDSKILSAFNTVIALLGSIVIIVMNIMIIQNYTRSTDNQAVIKDALQ | NivG.779 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRNNTVISRPGQSQCPRFNTCPAICAEGVYNDAFLIDRINWI | |
| | SAGVFLDSNATAANPVFTVFKDNEILYRAQLASEDTNAQKTITNCFLLKNKIW | |
| | CISLVEIYDTGDNVIRPKLFAVKIPEQCT | |
| 423 | MKKINEGLLDSKILSAFNTVIALLGSIVIIVMNIMIIQNYTRSTDNQAVIKDALQ | NivG.780 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRNNTVISRPGQSQCPRENTCPAICAEGVYNDAFLIDRINWI | |
| | SAGVFLDSQATAANPVFTVFKDNEILYRAQLASEDTNAQKTITNCFLLKNKIW | |
| | CISLVEIYDTGDNVIRPKLFAVKIPEQCT | |
| 424 | MKKINEGLLDSKILSAFNTVIALLGSIVIIVMNIMIIQNYTRSTDNQAVIKDALQ | NivG.781 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRQNTVISRPGQSQCPRFNTCPAICAEGVYNDAFLIDRINWI | |
| | SAGVFLDSNATAANPVFTVFKDNEILYRAQLASEDTNAQKTITNCFLLKNKIW | |
| | CISLVEIYDTGDNVIRPKLFAVKIPEQCT | |
| 425 | MKKINEGLLDSKILSAFNTVIALLGSIVIIVMNIMIIQNYTRSTDNQAVIKDALQ | NivG.782 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRQNTVISRPGQSQCPRFNTCPAICAEGVYNDAFLIDRINWI | |
| | SAGVFLDSQATAANPVFTVFKDNEILYRAQLASEDTNAQKTITNCFLLKNKIW | |
| | CISLVEIYDTGDNVIRPKLFAVKIPEQCT | |
| 426 | MKKINEGLLDSKILSAFNTVIALLGSIVIIVMNIMIIQNYTRSTDNQAVIKDALQ | NivG.783 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRNNTVISRPGQSQCPRFNTCPAICAEGVYNDAFLIDRINWI | |
| | SAGVELDSNATAANPVFTVFKDNEILYRAQLASEDTNAQKTITNCFLLKNKIW | |
| | CISLVEIYDTGDNVIRPKLFAVKIPEQCT | |
| 427 | MKKINEGLLDSKILSAFNTVIALLGSIVIIVMNIMIIQNYTRSTDNQAVIKDALQ | NivG.784 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRNNTVISRPGQSQCPRENTCPAICAEGVYNDAFLIDRINWI | |
| | SAGVFLDSQATAANPVFTVFKDNEILYRAQLASEDTNAQKTITNCFLLKNKIW | |
| | CISLVEIYDTGDNVIRPKLFAVKIPEQCT | |
| 428 | MKKINEGLLDSKILSAFNTVIALLGSIVIIVMNIMIIQNYTRSTDNQAVIKDALQ | NivG.785 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRQNTVISRPGQSQCPRFNTCPAICAEGVYNDAFLIDRINWI | |
| | SAGVFLDSNATAANPVFTVFKDNEILYRAQLASEDTNAQKTITNCFLLKNKIW | |
| | CISLVEIYDTGDNVIRPKLFAVKIPEQCT | |
| 429 | MKKINEGLLDSKILSAFNTVIALLGSIVIIVMNIMIIQNYTRSTDNQAVIKDALQ | NivG.786 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRQNTVISRPGQSQCPRFNTCPAICAEGVYNDAFLIDRINWI | |
| | SAGVFLDSQATAANPVFTVFKDNEILYRAQLASEDTNAQKTITNCFLLKNKIW | |
| | CISLVEIYDTGDNVIRPKLFAVKIPEQCT | |
| 430 | MKKINEGLLDSKILSAFNTVIALLGSIVIIVMNIMIIQNYTRSTDNQAVIKDALQ | NivG.787 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRNNTVISRPGQSQCPRFNTCPAICAEGVYNDAFLIDRINWI | |
| | SAGVELDSNATAANPVFTVFKDNEILYRAQLASEDTNAQKTITNCFLLKNKIW | |
| | CISLVEIYDTGDNVIRPKLFAVKIPEQCT | |
| 431 | MKKINEGLLDSKILSAFNTVIALLGSIVIIVMNIMIIQNYTRSTDNQAVIKDALQ | NivG.788 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRNNTVISRPGQSQCPRFNTCPAICAEGVYNDAFLIDRINWI | |
| | SAGVFLDSQATAANPVFTVFKDNEILYRAQLASEDTNAQKTITNCFLLKNKIW | |
| | CISLVEIYDTGDNVIRPKLFAVKIPEQCT | |
| 432 | MKKINEGLLDSKILSAFNTVIALLGSIVIIVMNIMIIQNYTRSTDNQAVIKDALQ | NivG.789 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRQNTVISRPGQSQCPRINTCPAICAEGVYNDAFLIDRINWI | |
| | SAGVELDSNATAANPVFTVFKDNEILYRAQLASEDTNAQKTITNCFLLKNKIW | |
| | CISLVEIYDTGDNVIRPKLFAVKIPEQCT | |
| 433 | MKKINEGLLDSKILSAFNTVIALLGSIVIIVMNIMIIQNYTRSTDNQAVIKDALQ | NivG.790 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRQNTVISRPGQSQCPRFNTCPAICAEGVYNDAFLIDRINWI | |
| | SAGVFLDSQATAANPVFTVFKDNEILYRAQLASEDTNAQKTITNCFLLKNKIW | |
| | CISLVEIYDTGDNVIRPKLFAVKIPEQCT | |
| 434 | MKKINEGLLDSKILSAFNTVIALLGSIVIIVMNIMIIQNYTRSTDNQAVIKDALQ | NivG.791 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRNNTVISRPGQSQCPRFNTCPAICAEGVYNDAFLIDRINWI | |
| | SAGVFLDSNATAANPVFTVFKDNEILYRAQLASEDTNAQKTITNCFLLKNKIW | |
| | CISLVEIYDTGDNVIRPKLFAVKIPEQCT | |
| 435 | MKKINEGLLDSKILSAFNTVIALLGSIVIIVMNIMIIQNYTRSTDNQAVIKDALQ | NivG.792 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRNNTVISRPGQSQCPRFNTCPAICAEGVYNDAFLIDRINWI | |
| | SAGVELDSQATAANPVFTVFKDNEILYRAQLASEDTNAQKTITNCFLLKNKIW | |
| | CISLVEIYDTGDNVIRPKLFAVKIPEQCT | |
| 436 | MKKINEGLLDSKILSAFNTVIALLGSIVIIVMNIMIIQNYTRSTDNQAVIKDALQ | NivG.793 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRQNTVISRPGQSQCPRINTCPAICAEGVYNDAFLIDRINWI | |
| | SAGVELDSNATAANPVFTVFKDNEILYRAQLASEDTNAQKTITNCFLLKNKIW | |
| | CISLVEIYDTGDNVIRPKLFAVKIPEQCT | |
| 437 | MKKINEGLLDSKILSAFNTVIALLGSIVIIVMNIMIIQNYTRSTDNQAVIKDALQ | NivG.794 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRQNTVISRPGQSQCPRFNTCPAICAEGVYNDAFLIDRINWI | |
| | SAGVFLDSQATAANPVFTVFKDNEILYRAQLASEDTNAQKTITNCFLLKNKIW | |
| | CISLVEIYDTGDNVIRPKLFAVKIPEQCT | |
| 438 | MKKINEGLLDSKILSAFNTVIALLGSIVIIVMNIMIIQNYTRSTDNQAVIKDALQ | NivG.795 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRNNTVISRPGQSQCPRFNTCPAICAEGVYNDAFLIDRINWI | |
| | SAGVFLDSNATAANPVFTVFKDNEILYRAQLASEDTNAQKTITNCFLLKNKIW | |
| | CISLVEIYDTGDNVIRPKLFAVKIPEQCT | |
| 439 | MKKINEGLLDSKILSAFNTVIALLGSIVIIVMNIMIIQNYTRSTDNQAVIKDALQ | NivG.796 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRNNTVISRPGQSQCPRFNTCPAICAEGVYNDAFLIDRINWI | |
| | SAGVELDSQATAANPVFTVFKDNEILYRAQLASEDTNAQKTITNCFLLKNKIW | |
| | CISLVEIYDTGDNVIRPKLFAVKIPEQCT | |
| 440 | MKKINEGLLDSKILSAFNTVIALLGSIVIIVMNIMIIQNYTRSTDNQAVIKDALQ | NivG.797 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRQNTVISRPGQSQCPRFNTCPAICAEGVYNDAFLIDRINWI | |
| | SAGVFLDSNATAANPVFTVFKDNEILYRAQLASEDTNAQKTITNCFLLKNKIW | |
| | CISLVEIYDTGDNVIRPKLFAVKIPEQCT | |
| 441 | MKKINEGLLDSKILSAFNTVIALLGSIVIIVMNIMIIQNYTRSTDNQAVIKDALQ | NivG.798 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRQNTVISRPGQSQCPRENTCPAICAEGVYNDAFLIDRINWI | |
| | SAGVFLDSQATAANPVFTVFKDNEILYRAQLASEDTNAQKTITNCFLLKNKIW | |
| | CISLVEIYDTGDNVIRPKLFAVKIPEQCT | |
| 442 | MKKINEGLLDSKILSAFNTVIALLGSIVIIVMNIMIIQQYTRSTDNQAVIKDALQ | NivG.799 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRNNTVISRPGQSQCPRFNTCPAICAEGVYNDAFLIDRINWI | |
| | SAGVFLDSQATAANPVFTVFKDNEILYRAQLASEDTNAQKTITNCFLLKNKIW | |
| | CISLVEIYDTGDNVIRPKLFAVKIPEQCT | |
| 443 | MKKINEGLLDSKILSAFNTVIALLGSIVIIVMNIMIIQQYTRSTDNQAVIKDALQ | NivG.800 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRQNTVISRPGQSQCPRFNTCPAICAEGVYNDAFLIDRINWI | |
| | SAGVFLDSNATAANPVFTVFKDNEILYRAQLASEDTNAQKTITNCFLLKNKIW | |
| | CISLVEIYDTGDNVIRPKLFAVKIPEQCT | |
| 444 | MKKINEGLLDSKILSAFNTVIALLGSIVIIVMNIMIIQQYTRSTDNQAVIKDALQ | NivG.801 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRQNTVISRPGQSQCPRFNTCPAICAEGVYNDAFLIDRINWI | |
| | SAGVELDSQATAANPVFTVFKDNEILYRAQLASEDTNAQKTITNCFLLKNKIW | |
| | CISLVEIYDTGDNVIRPKLFAVKIPEQCT | |
| 445 | MKKINEGLLDSKILSAFNTVIALLGSIVIIVMNIMIIQQYTRSTDNQAVIKDALQ | NivG.802 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRNNTVISRPGQSQCPRENTCPAICAEGVYNDAFLIDRINWI | |
| | SAGVELDSNATAANPVFTVFKDNEILYRAQLASEDTNAQKTITNCFLLKNKIW | |
| | CISLVEIYDTGDNVIRPKLFAVKIPEQCT | |
| 446 | MKKINEGLLDSKILSAFNTVIALLGSIVIIVMNIMIIQQYTRSTDNQAVIKDALQ | NivG.803 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRNNTVISRPGQSQCPRFNTCPAICAEGVYNDAFLIDRINWI | |
| | SAGVFLDSQATAANPVFTVFKDNEILYRAQLASEDTNAQKTITNCFLLKNKIW | |
| | CISLVEIYDTGDNVIRPKLFAVKIPEQCT | |
| 447 | MKKINEGLLDSKILSAFNTVIALLGSIVIIVMNIMIIQQYTRSTDNQAVIKDALQ | NivG.804 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRQNTVISRPGQSQCPRFNTCPAICAEGVYNDAFLIDRINWI | |
| | SAGVFLDSNATAANPVFTVFKDNEILYRAQLASEDTNAQKTITNCFLLKNKIW | |
| | CISLVEIYDTGDNVIRPKLFAVKIPEQCT | |
| 448 | MKKINEGLLDSKILSAFNTVIALLGSIVIIVMNIMIIQQYTRSTDNQAVIKDALQ | NivG.805 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRQNTVISRPGQSQCPRFNTCPAICAEGVYNDAFLIDRINWI | |
| | SAGVELDSQATAANPVFTVFKDNEILYRAQLASEDTNAQKTITNCFLLKNKIW | |
| | CISLVEIYDTGDNVIRPKLFAVKIPEQCT | |
| 449 | MKKINEGLLDSKILSAFNTVIALLGSIVIIVMNIMIIQQYTRSTDNQAVIKDALQ | NivG.806 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRNNTVISRPGQSQCPRFNTCPAICAEGVYNDAFLIDRINWI | |
| | SAGVELDSNATAANPVFTVFKDNEILYRAQLASEDTNAQKTITNCFLLKNKIW | |
| | CISLVEIYDTGDNVIRPKLFAVKIPEQCT | |
| 450 | MKKINEGLLDSKILSAFNTVIALLGSIVIIVMNIMIIQQYTRSTDNQAVIKDALQ | NivG.807 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRNNTVISRPGQSQCPRENTCPAICAEGVYNDAFLIDRINWI | |
| | SAGVFLDSQATAANPVFTVFKDNEILYRAQLASEDTNAQKTITNCFLLKNKIW | |
| | CISLVEIYDTGDNVIRPKLFAVKIPEQCT | |
| 451 | MKKINEGLLDSKILSAFNTVIALLGSIVIIVMNIMIIQQYTRSTDNQAVIKDALQ | NivG.808 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRQNTVISRPGQSQCPRFNTCPAICAEGVYNDAFLIDRINWI | |
| | SAGVELDSNATAANPVFTVFKDNEILYRAQLASEDTNAQKTITNCFLLKNKIW | |
| | CISLVEIYDTGDNVIRPKLFAVKIPEQCT | |
| 452 | MKKINEGLLDSKILSAFNTVIALLGSIVIIVMNIMIIQQYTRSTDNQAVIKDALQ | NivG.809 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRQNTVISRPGQSQCPRFNTCPAICAEGVYNDAFLIDRINWI | |
| | SAGVFLDSQATAANPVFTVFKDNEILYRAQLASEDTNAQKTITNCFLLKNKIW | |
| | CISLVEIYDTGDNVIRPKLFAVKIPEQCT | |
| 453 | MKKINEGLLDSKILSAFNTVIALLGSIVIIVMNIMIIQQYTRSTDNQAVIKDALQ | NivG.810 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRNNTVISRPGQSQCPRFNTCPAICAEGVYNDAFLIDRINWI | |
| | SAGVFLDSNATAANPVFTVFKDNEILYRAQLASEDTNAQKTITNCFLLKNKIW | |
| | CISLVEIYDTGDNVIRPKLFAVKIPEQCT | |
| 454 | MKKINEGLLDSKILSAFNTVIALLGSIVIIVMNIMIIQQYTRSTDNQAVIKDALQ | NivG.811 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRNNTVISRPGQSQCPRENTCPAICAEGVYNDAFLIDRINWI | |
| | SAGVFLDSQATAANPVFTVFKDNEILYRAQLASEDTNAQKTITNCFLLKNKIW | |
| | CISLVEIYDTGDNVIRPKLFAVKIPEQCT | |
| 455 | MKKINEGLLDSKILSAFNTVIALLGSIVIIVMNIMIIQQYTRSTDNQAVIKDALQ | NivG.812 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRQNTVISRPGQSQCPRFNTCPAICAEGVYNDAFLIDRINWI | |
| | SAGVFLDSNATAANPVFTVFKDNEILYRAQLASEDTNAQKTITNCFLLKNKIW | |
| | CISLVEIYDTGDNVIRPKLFAVKIPEQCT | |
| 456 | MKKINEGLLDSKILSAFNTVIALLGSIVIIVMNIMIIQQYTRSTDNQAVIKDALQ | NivG.813 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRQNTVISRPGQSQCPRFNTCPAICAEGVYNDAFLIDRINWI | |
| | SAGVFLDSQATAANPVFTVFKDNEILYRAQLASEDTNAQKTITNCFLLKNKIW | |
| | CISLVEIYDTGDNVIRPKLFAVKIPEQCT | |
| 457 | MKKINEGLLDSKILSAFNTVIALLGSIVIIVMNIMIIQQYTRSTDNQAVIKDALQ | NivG.814 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRNNTVISRPGQSQCPRFNTCPAICAEGVYNDAFLIDRINWI | |
| | SAGVELDSNATAANPVFTVFKDNEILYRAQLASEDTNAQKTITNCFLLKNKIW | |
| | CISLVEIYDTGDNVIRPKLFAVKIPEQCT | |
| 458 | MKKINEGLLDSKILSAFNTVIALLGSIVIIVMNIMIIQQYTRSTDNQAVIKDALQ | NivG.815 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRNNTVISRPGQSQCPRFNTCPAICAEGVYNDAFLIDRINWI | |
| | SAGVFLDSQATAANPVFTVFKDNEILYRAQLASEDTNAQKTITNCFLLKNKIW | |
| | CISLVEIYDTGDNVIRPKLFAVKIPEQCT | |
| 459 | MKKINEGLLDSKILSAFNTVIALLGSIVIIVMNIMIIQQYTRSTDNQAVIKDALQ | NivG.816 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRQNTVISRPGQSQCPRENTCPAICAEGVYNDAFLIDRINWI | |
| | SAGVELDSNATAANPVFTVFKDNEILYRAQLASEDTNAQKTITNCFLLKNKIW | |
| | CISLVEIYDTGDNVIRPKLFAVKIPEQCT | |
| 460 | MKKINEGLLDSKILSAFNTVIALLGSIVIIVMNIMIIQQYTRSTDNQAVIKDALQ | NivG.817 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRQNTVISRPGQSQCPRFNTCPAICAEGVYNDAFLIDRINWI | |
| | SAGVFLDSQATAANPVFTVFKDNEILYRAQLASEDTNAQKTITNCFLLKNKIW | |
| | CISLVEIYDTGDNVIRPKLFAVKIPEQCT | |
| 461 | MKKINEGLLDSKILSAFNTVIALLGSIVIIVMNIMIIQQYTRSTDNQAVIKDALQ | NivG.818 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRNNTVISRPGQSQCPRFNTCPAICAEGVYNDAFLIDRINWI | |
| | SAGVELDSNATAANPVFTVFKDNEILYRAQLASEDTNAQKTITNCFLLKNKIW | |
| | CISLVEIYDTGDNVIRPKLFAVKIPEQCT | |
| 462 | MKKINEGLLDSKILSAFNTVIALLGSIVIIVMNIMIIQQYTRSTDNQAVIKDALQ | NivG.819 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRNNTVISRPGQSQCPRFNTCPAICAEGVYNDAFLIDRINWI | |
| | SAGVELDSQATAANPVFTVFKDNEILYRAQLASEDTNAQKTITNCFLLKNKIW | |
| | CISLVEIYDTGDNVIRPKLFAVKIPEQCT | |
| 463 | MKKINEGLLDSKILSAFNTVIALLGSIVIIVMNIMIIQQYTRSTDNQAVIKDALQ | NivG.820 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRQNTVISRPGQSQCPRENTCPAICAEGVYNDAFLIDRINWI | |
| | SAGVFLDSNATAANPVFTVFKDNEILYRAQLASEDTNAQKTITNCFLLKNKIW | |
| | CISLVEIYDTGDNVIRPKLFAVKIPEQCT | |
| 464 | MKKINEGLLDSKILSAFNTVIALLGSIVIIVMNIMIIQQYTRSTDNQAVIKDALQ | NivG.821 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRQNTVISRPGQSQCPRFNTCPAICAEGVYNDAFLIDRINWI | |
| | SAGVFLDSQATAANPVFTVFKDNEILYRAQLASEDTNAQKTITNCFLLKNKIW | |
| | CISLVEIYDTGDNVIRPKLFAVKIPEQCT | |
| 465 | MKKINEGLLDSKILSAFNTVIALLGSIVIIVMNIMIIQQYTRSTDNQAVIKDALQ | NivG.822 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRNNTVISRPGQSQCPRFNTCPAICAEGVYNDAFLIDRINWI | |
| | SAGVFLDSNATAANPVFTVFKDNEILYRAQLASEDTNAQKTITNCFLLKNKIW | |
| | CISLVEIYDTGDNVIRPKLFAVKIPEQCT | |
| 466 | MKKINEGLLDSKILSAFNTVIALLGSIVIIVMNIMIIQQYTRSTDNQAVIKDALQ | NivG.823 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRNNTVISRPGQSQCPRFNTCPAICAEGVYNDAFLIDRINWI | |
| | SAGVELDSQATAANPVFTVFKDNEILYRAQLASEDTNAQKTITNCFLLKNKIW | |
| | CISLVEIYDTGDNVIRPKLFAVKIPEQCT | |
| 467 | MKKINEGLLDSKILSAFNTVIALLGSIVIIVMNIMIIQQYTRSTDNQAVIKDALQ | NivG.824 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRQNTVISRPGQSQCPRFNTCPAICAEGVYNDAFLIDRINWI | |
| | SAGVFLDSNATAANPVFTVFKDNEILYRAQLASEDTNAQKTITNCFLLKNKIW | |
| | CISLVEIYDTGDNVIRPKLFAVKIPEQCT | |
| 468 | MKKINEGLLDSKILSAFNTVIALLGSIVIIVMNIMIIQQYTRSTDNQAVIKDALQ | NivG.825 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRQNTVISRPGQSQCPRENTCPAICAEGVYNDAFLIDRINWI | |
| | SAGVFLDSQATAANPVFTVFKDNEILYRAQLASEDTNAQKTITNCFLLKNKIW | |
| | CISLVEIYDTGDNVIRPKLFAVKIPEQCT | |
| 469 | MKKINEGLLDSKILSAFNTVIALLGSIVIIVMNIMIIQQYTRSTDNQAVIKDALQ | NivG.826 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRNNTVISRPGQSQCPRFNTCPAICAEGVYNDAFLIDRINWI | |
| | SAGVELDSNATAANPVFTVFKDNEILYRAQLASEDTNAQKTITNCFLLKNKIW | |
| | CISLVEIYDTGDNVIRPKLFAVKIPEQCT | |
| 470 | MKKINEGLLDSKILSAFNTVIALLGSIVIIVMNIMIIQQYTRSTDNQAVIKDALQ | NivG.827 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRNNTVISRPGQSQCPRFNTCPAICAEGVYNDAFLIDRINWI | |
| | SAGVFLDSQATAANPVFTVFKDNEILYRAQLASEDTNAQKTITNCFLLKNKIW | |
| | CISLVEIYDTGDNVIRPKLFAVKIPEQCT | |
| 471 | MKKINEGLLDSKILSAFNTVIALLGSIVIIVMNIMIIQQYTRSTDNQAVIKDALQ | NivG.828 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRQNTVISRPGQSQCPRFNTCPAICAEGVYNDAFLIDRINWI | |
| | SAGVELDSNATAANPVFTVFKDNEILYRAQLASEDTNAQKTITNCFLLKNKIW | |
| | CISLVEIYDTGDNVIRPKLFAVKIPEQCT | |
| 472 | MKKINEGLLDSKILSAFNTVIALLGSIVIIVMNIMIIQQYTRSTDNQAVIKDALQ | NivG.829 |
| | GIQQQIKGLADKIGTEIGPKVSLIDTSSTITIPANIGLLGSKISQSTASINENVNEK | |
| | CKFTLPPLKIHECNISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRQNTVISRPGQSQCPRENTCPAICAEGVYNDAFLIDRINWI | |
| | SAGVFLDSQATAANPVFTVFKDNEILYRAQLASEDTNAQKTITNCFLLKNKIW | |
| | CISLVEIYDTGDNVIRPKLFAVKIPEQCT | |
| 473 | MKKINEGLLDSKILSAFNTVIALLGSIVIIVMNIMIIQQYTRSTDNQAVIKDALQ | NivG.830 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRNNTVISRPGQSQCPRFNTCPAICAEGVYNDAFLIDRINWI | |
| | SAGVELDSNATAANPVFTVFKDNEILYRAQLASEDTNAQKTITNCFLLKNKIW | |
| | CISLVEIYDTGDNVIRPKLFAVKIPEQCT | |
| 474 | MKKINEGLLDSKILSAFNTVIALLGSIVIIVMNIMIIQQYTRSTDNQAVIKDALQ | NivG.831 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRNNTVISRPGQSQCPRFNTCPAICAEGVYNDAFLIDRINWI | |
| | SAGVFLDSQATAANPVFTVFKDNEILYRAQLASEDTNAQKTITNCFLLKNKIW | |
| | CISLVEIYDTGDNVIRPKLFAVKIPEQCT | |
| 475 | MKKINEGLLDSKILSAFNTVIALLGSIVIIVMNIMIIQQYTRSTDNQAVIKDALQ | NivG.832 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRQNTVISRPGQSQCPRFNTCPAICAEGVYNDAFLIDRINWI | |
| | SAGVELDSNATAANPVFTVFKDNEILYRAQLASEDTNAQKTITNCFLLKNKIW | |
| | CISLVEIYDTGDNVIRPKLFAVKIPEQCT | |
| 476 | MKKINEGLLDSKILSAFNTVIALLGSIVIIVMNIMIIQQYTRSTDNQAVIKDALQ | NivG.833 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRQNTVISRPGQSQCPRFNTCPAICAEGVYNDAFLIDRINWI | |
| | SAGVFLDSQATAANPVFTVFKDNEILYRAQLASEDTNAQKTITNCFLLKNKIW | |
| | CISLVEIYDTGDNVIRPKLFAVKIPEQCT | |
| 477 | MKKINEGLLDSKILSAFNTVIALLGSIVIIVMNIMIIQQYTRSTDNQAVIKDALQ | NivG.834 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRNNTVISRPGQSQCPRENTCPAICAEGVYNDAFLIDRINWI | |
| | SAGVFLDSNATAANPVFTVFKDNEILYRAQLASEDTNAQKTITNCFLLKNKIW | |
| | CISLVEIYDTGDNVIRPKLFAVKIPEQCT | |
| 478 | MKKINEGLLDSKILSAFNTVIALLGSIVIIVMNIMIIQQYTRSTDNQAVIKDALQ | NivG.835 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRNNTVISRPGQSQCPRFNTCPAICAEGVYNDAFLIDRINWI | |
| | SAGVFLDSQATAANPVFTVFKDNEILYRAQLASEDTNAQKTITNCFLLKNKIW | |
| | CISLVEIYDTGDNVIRPKLFAVKIPEQCT | |
| 479 | MKKINEGLLDSKILSAFNTVIALLGSIVIIVMNIMIIQQYTRSTDNQAVIKDALQ | NivG.836 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRQNTVISRPGQSQCPRFNTCPAICAEGVYNDAFLIDRINWI | |
| | SAGVFLDSNATAANPVFTVFKDNEILYRAQLASEDTNAQKTITNCFLLKNKIW | |
| | CISLVEIYDTGDNVIRPKLFAVKIPEQCT | |
| 480 | MKKINEGLLDSKILSAFNTVIALLGSIVIIVMNIMIIQQYTRSTDNQAVIKDALQ | NivG.837 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRQNTVISRPGQSQCPRFNTCPAICAEGVYNDAFLIDRINWI | |
| | SAGVELDSQATAANPVFTVFKDNEILYRAQLASEDTNAQKTITNCFLLKNKIW | |
| | CISLVEIYDTGDNVIRPKLFAVKIPEQCT | |
| 481 | MKKINEGLLDSKILSAFNTVIALLGSIVIIVMNIMIIQQYTRSTDNQAVIKDALQ | NivG.838 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRNNTVISRPGQSQCPRINTCPAICAEGVYNDAFLIDRINWI | |
| | SAGVFLDSNATAANPVFTVFKDNEILYRAQLASEDTNAQKTITNCFLLKNKIW | |
| | CISLVEIYDTGDNVIRPKLFAVKIPEQCT | |
| 482 | MKKINEGLLDSKILSAFNTVIALLGSIVIIVMNIMIIQQYTRSTDNQAVIKDALQ | NivG.839 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRNNTVISRPGQSQCPRFNTCPAICAEGVYNDAFLIDRINWI | |
| | SAGVFLDSQATAANPVFTVFKDNEILYRAQLASEDTNAQKTITNCFLLKNKIW | |
| | CISLVEIYDTGDNVIRPKLFAVKIPEQCT | |
| 483 | MKKINEGLLDSKILSAFNTVIALLGSIVIIVMNIMIIQQYTRSTDNQAVIKDALQ | NivG.840 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRQNTVISRPGQSQCPRFNTCPAICAEGVYNDAFLIDRINWI | |
| | SAGVFLDSNATAANPVFTVFKDNEILYRAQLASEDTNAQKTITNCFLLKNKIW | |
| | CISLVEIYDTGDNVIRPKLFAVKIPEQCT | |
| 484 | MKKINEGLLDSKILSAFNTVIALLGSIVIIVMNIMIIQQYTRSTDNQAVIKDALQ | NivG.841 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRQNTVISRPGQSQCPRFNTCPAICAEGVYNDAFLIDRINWI | |
| | SAGVELDSQATAANPVFTVFKDNEILYRAQLASEDTNAQKTITNCFLLKNKIW | |
| | CISLVEIYDTGDNVIRPKLFAVKIPEQCT | |
| 485 | MKKINEGLLDSKILSAFNTVIALLGSIVIIVMNIMIIQQYTRSTDNQAVIKDALQ | NivG.842 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRNNTVISRPGQSQCPRFNTCPAICAEGVYNDAFLIDRINWI | |
| | SAGVFLDSNATAANPVFTVFKDNEILYRAQLASEDTNAQKTITNCFLLKNKIW | |
| | CISLVEIYDTGDNVIRPKLFAVKIPEQCT | |
| 486 | MKKINEGLLDSKILSAFNTVIALLGSIVIIVMNIMIIQQYTRSTDNQAVIKDALQ | NivG.843 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRNNTVISRPGQSQCPRENTCPAICAEGVYNDAFLIDRINWI | |
| | SAGVFLDSQATAANPVFTVFKDNEILYRAQLASEDTNAQKTITNCFLLKNKIW | |
| | CISLVEIYDTGDNVIRPKLFAVKIPEQCT | |
| 487 | MKKINEGLLDSKILSAFNTVIALLGSIVIIVMNIMIIQQYTRSTDNQAVIKDALQ | NivG.844 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRQNTVISRPGQSQCPRFNTCPAICAEGVYNDAFLIDRINWI | |
| | SAGVFLDSNATAANPVFTVFKDNEILYRAQLASEDTNAQKTITNCFLLKNKIW | |
| | CISLVEIYDTGDNVIRPKLFAVKIPEQCT | |
| 488 | MKKINEGLLDSKILSAFNTVIALLGSIVIIVMNIMIIQQYTRSTDNQAVIKDALQ | NivG.845 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILN | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRQNTVISRPGQSQCPRFNTCPAICAEGVYNDAFLIDRINWI | |
| | SAGVFLDSQATAANPVFTVFKDNEILYRAQLASEDTNAQKTITNCFLLKNKIW | |
| | CISLVEIYDTGDNVIRPKLFAVKIPEQCT | |
| 489 | MKKINEGLLDSKILSAFNTVIALLGSIVIIVMNIMIIQQYTRSTDNQAVIKDALQ | NivG.846 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRNNTVISRPGQSQCPRFNTCPAICAEGVYNDAFLIDRINWI | |
| | SAGVELDSNATAANPVFTVFKDNEILYRAQLASEDTNAQKTITNCFLLKNKIW | |
| | CISLVEIYDTGDNVIRPKLFAVKIPEQCT | |
| 490 | MKKINEGLLDSKILSAFNTVIALLGSIVIIVMNIMIIQQYTRSTDNQAVIKDALQ | NivG.847 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRNNTVISRPGQSQCPRENTCPAICAEGVYNDAFLIDRINWI | |
| | SAGVFLDSQATAANPVFTVFKDNEILYRAQLASEDTNAQKTITNCFLLKNKIW | |
| | CISLVEIYDTGDNVIRPKLFAVKIPEQCT | |
| 491 | MKKINEGLLDSKILSAFNTVIALLGSIVIIVMNIMIIQQYTRSTDNQAVIKDALQ | NivG.848 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRQNTVISRPGQSQCPRFNTCPAICAEGVYNDAFLIDRINWI | |
| | SAGVFLDSNATAANPVFTVFKDNEILYRAQLASEDTNAQKTITNCFLLKNKIW | |
| | CISLVEIYDTGDNVIRPKLFAVKIPEQCT | |
| 492 | MKKINEGLLDSKILSAFNTVIALLGSIVIIVMNIMIIQQYTRSTDNQAVIKDALQ | NivG.849 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRQNTVISRPGQSQCPRFNTCPAICAEGVYNDAFLIDRINWI | |
| | SAGVFLDSQATAANPVFTVFKDNEILYRAQLASEDTNAQKTITNCFLLKNKIW | |
| | CISLVEIYDTGDNVIRPKLFAVKIPEQCT | |
| 493 | MKKINEGLLDSKILSAFNTVIALLGSIVIIVMNIMIIQQYTRSTDNQAVIKDALQ | NivG.850 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRNNTVISRPGQSQCPRFNTCPAICAEGVYNDAFLIDRINWI | |
| | SAGVELDSNATAANPVFTVFKDNEILYRAQLASEDTNAQKTITNCFLLKNKIW | |
| | CISLVEIYDTGDNVIRPKLFAVKIPEQCT | |
| 494 | MKKINEGLLDSKILSAFNTVIALLGSIVIIVMNIMIIQQYTRSTDNQAVIKDALQ | NivG.851 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRNNTVISRPGQSQCPRFNTCPAICAEGVYNDAFLIDRINWI | |
| | SAGVFLDSQATAANPVFTVFKDNEILYRAQLASEDTNAQKTITNCFLLKNKIW | |
| | CISLVEIYDTGDNVIRPKLFAVKIPEQCT | |
| 495 | MKKINEGLLDSKILSAFNTVIALLGSIVIIVMNIMIIQQYTRSTDNQAVIKDALQ | NivG.852 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRQNTVISRPGQSQCPRENTCPAICAEGVYNDAFLIDRINWI | |
| | SAGVFLDSNATAANPVFTVFKDNEILYRAQLASEDTNAQKTITNCFLLKNKIW | |
| | CISLVEIYDTGDNVIRPKLFAVKIPEQCT | |
| 496 | MKKINEGLLDSKILSAFNTVIALLGSIVIIVMNIMIIQQYTRSTDNQAVIKDALQ | NivG.853 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYNDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRQNTVISRPGQSQCPRFNTCPAICAEGVYNDAFLIDRINWI | |
| | SAGVFLDSQATAANPVFTVFKDNEILYRAQLASEDTNAQKTITNCFLLKNKIW | |
| | CISLVEIYDTGDNVIRPKLFAVKIPEQCT | |
| 497 | MKKINEGLLDSKILSAFNTVIALLGSIVIIVMNIMIIQQYTRSTDNQAVIKDALQ | NivG.854 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRNNTVISRPGQSQCPRFNTCPAICAEGVYNDAFLIDRINWI | |
| | SAGVFLDSNATAANPVFTVFKDNEILYRAQLASEDTNAQKTITNCFLLKNKIW | |
| | CISLVEIYDTGDNVIRPKLFAVKIPEQCT | |
| 498 | MKKINEGLLDSKILSAFNTVIALLGSIVIIVMNIMIIQQYTRSTDNQAVIKDALQ | NivG.855 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRNNTVISRPGQSQCPRFNTCPAICAEGVYNDAFLIDRINWI | |
| | SAGVELDSQATAANPVFTVFKDNEILYRAQLASEDTNAQKTITNCFLLKNKIW | |
| | CISLVEIYDTGDNVIRPKLFAVKIPEQCT | |
| 499 | MKKINEGLLDSKILSAFNTVIALLGSIVIIVMNIMIIQQYTRSTDNQAVIKDALQ | NivG.856 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRQNTVISRPGQSQCPRENTCPAICAEGVYNDAFLIDRINWI | |
| | SAGVFLDSNATAANPVFTVFKDNEILYRAQLASEDTNAQKTITNCFLLKNKIW | |
| | CISLVEIYDTGDNVIRPKLFAVKIPEQCT | |
| 500 | MKKINEGLLDSKILSAFNTVIALLGSIVIIVMNIMIIQQYTRSTDNQAVIKDALQ | NivG.857 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYNLSDGENPKVVFIEISDQRLSIGSPSKIYDSLGQPVFYQASFSWDTMIKF | |
| | GDVLTVNPLVVNWRQNTVISRPGQSQCPRFNTCPAICAEGVYNDAFLIDRINWI | |
| | SAGVFLDSQATAANPVFTVFKDNEILYRAQLASEDTNAQKTITNCFLLKNKIW | |
| | CISLVEIYDTGDNVIRPKLFAVKIPEQCT | |
| 501 | MKKINEGLLDSKILSAFNTVIALLGSIVIIVMNIMIIQQYTRSTDNQAVIKDALQ | NivG.858 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRNNTVISRPGQSQCPRFNTCPAICAEGVYNDAFLIDRINWI | |
| | SAGVFLDSNATAANPVFTVFKDNEILYRAQLASEDTNAQKTITNCFLLKNKIW | |
| | CISLVEIYDTGDNVIRPKLFAVKIPEQCT | |
| 502 | MKKINEGLLDSKILSAFNTVIALLGSIVIIVMNIMIIQQYTRSTDNQAVIKDALQ | NivG.859 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRNNTVISRPGQSQCPRFNTCPAICAEGVYNDAFLIDRINWI | |
| | SAGVELDSQATAANPVFTVFKDNEILYRAQLASEDTNAQKTITNCFLLKNKIW | |
| | CISLVEIYDTGDNVIRPKLFAVKIPEQCT | |
| 503 | MKKINEGLLDSKILSAFNTVIALLGSIVIIVMNIMIIQQYTRSTDNQAVIKDALQ | NivG.860 |
| | GIQQQIKGLADKIGTEIGPKVSLIDTSSTITIPANIGLLGSKISQSTASINENVNEK | |
| | CKFTLPPLKIHECQISCPNPLPFREYRPQTEGVSNLVGLPNNICLQKTSNQILKP | |
| | KLISYTLPVVGQSGTCITDPLLAMDEGYFAYSHLERIGSCSRGVSKQRIIGVGE | |
| | VLDRGDEVPSLFMTNVWTPPNPNTVYHCSAVYNNEFYYVLCAVSTVGDPILQ | |
| | STYWSGSLMMTRLAVKPKSNGGGYNQHQLALRSIEKGRYDKVMPYGPSGIK | |
| | QGDTLYFPAVGFLVRTEFKYQDSNCPITKCQYSKPENCRLSMGIRPNSHYILRS | |
| | GLLKYQLSDGENPKVVFIEISDQRLSIGSPSKIYDSLGQPVFYQASFSWDTMIKF | |
| | GDVLTVNPLVVNWRQNTVISRPGQSQCPRFNTCPAICAEGVYNDAFLIDRINWI | |
| | SAGVFLDSNATAANPVFTVFKDNEILYRAQLASEDTNAQKTITNCFLLKNKIW | |
| | CISLVEIYDTGDNVIRPKLFAVKIPEQCT | |
| 504 | GYTFTSYY | GPRC5D |
| | | Binder 1- |
| | | HCDR1 |
| 505 | GFTFSSYA | GPRC5D |
| | | Binder 2- |
| | | HCDR1 |
| 506 | GDSVSSNSAA | GPRC5D |
| | | Binder 3- |
| | | HCDR1 |
| 507 | INPNSGGT | GPRC5D |
| | | Binder 1- |
| | | HCDR2 |
| 508 | ISGSGGST | GPRC5D |
| | | Binder 2- |
| | | HCDR2 |
| 509 | TYYRSKWYN | GPRC5D |
| | | Binder 3- |
| | | HCDR2 |
| 510 | VRSKGRAARNYYYMDV | GPRC5D |
| | | Binder 1- |
| | | HCDR3 |
| 511 | ARDLYGYRYYYYGMDV | GPRC5D |
| | | Binder 2- |
| | | HCDR3 |
| 512 | ARAYSPSRLRWSRAAAFDI | GPRC5D |
| | | Binder 3- |
| | | HCDR3 |
| 513 | SVRTYY | GPRC5D |
| | | Binder 1- |
| | | LCDR1 |
| 514 | SLRRYF | GPRC5D |
| | | Binder 2- |
| | | LCDR1 |
| 515 | SLRSYY | GPRC5D |
| | | Binder 3- |
| | | LCDR1 |
| 516 | GKN | GPRC5D |
| | | Binder 1- |
| | | LCDR2 |
| 517 | GKN | GPRC5D |
| | | Binder 2- |
| | | LCDR2 |
| 518 | GKN | GPRC5D |
| | | Binder 3- |
| | | LCDR2 |
| 519 | NSRDSSANPV | GPRC5D |
| | | Binder 1- |
| | | LCDR3 |
| 520 | NSRDRSGTVV | GPRC5D |
| | | Binder 2- |
| | | LCDR3 |
| 521 | SSRDSSGNHLVV | GPRC5D |
| | | Binder 3- |
| | | LCDR3 |
| 522 | QVQLVQSGAEVKKPGASVKVSCKASGYTFTSYYMHWVRQAPGQGLEWMG | GPRC5D |
| | WINPNSGGTNYAQKFQGRVTITADKSTSTAYMELNSLRAEDTAVYYCVRSKGR | Binder 1-VH |
| | AARNYYYMDVWGKGTTVTVSS | |
| 523 | EVQLVESGGGVVQPGRSLRLSCAASGFTFSSYAMSWVRQAPGKGLEWVSAIS | GPRC5D |
| | GSGGSTYYAGSVKGRFTISRDNSKNTLYLQMNSLRAEDTAVYYCARDLYGYR | Binder 2-VH |
| | YYYYGMDVWGQGTMVTVSS | |
| 524 | QVQLQQSGPGQVKPSQTLSLTCAISGDSVSSNSAAWNWIRQSPSRGLEWLGRT | GPRC5D |
| | YYRSKWYNDYAVSVKSRITINPDTSKNQFSLQLNSVTPEDTAVYYCARAYSPS | Binder 3-VH |
| | RLRWSRAAAFDIWGQGTTVTVSS | |
| 525 | SSELTQDPAASVALGQTVRITCQGDSVRTYYAGWYQQKPGQAPVLVIYGKNH | GPRC5D |
| | RPSGIPDRESGSTSGNTASLTITGVQAEDEADYYCNSRDSSANPVFGGGTKVTV | Binder 1-VL |
| | L | |
| 526 | SSELTQDPAASVALGQTVKITCQGDSLRRYFASWYQQKPGQAPTLVTYGKNRR | GPRC5D |
| | PSGVPDRLSGSSSGDTASLTITGAQAEDEGDYYCNSRDRSGTVVFGGGTKLTV | Binder 2-VL |
| | L | |
| 527 | SSELTQDPAVSVALGQTVRITCQGDSLRSYYANWYQQKPGQAPILVNYGKNNR | GPRC5D |
| | PSGIPDRFSGSSSGKTASLTITGAQAEDEADYYCSSRDSSGNHLVVFGGGTQLT | Binder 3-VL |
| | VL | |
| 528 | SSELTQDPAASVALGQTVRITCQGDSVRTYYAGWYQQKPGQAPVLVIYGKNH | GPRC5D |
| | RPSGIPDRFSGSTSGNTASLTITGVQAEDEADYYCNSRDSSANPVFGGGTKVTV | Binder 1 |
| | LGGGGSGGGGSGGGGSQVQLVQSGAEVKKPGASVKVSCKASGYTFTSYYMH | |
| | WVRQAPGQGLEWMGWINPNSGGTNYAQKFQGRVTITADKSTSTAYMELNSL | |
| | RAEDTAVYYCVRSKGRAARNYYYMDVWGKGTTVTVSS | |
| 529 | SSELTQDPAASVALGQTVKITCQGDSLRRYFASWYQQKPGQAPTLVTYGKNRR | GPRC5D |
| | PSGVPDRLSGSSSGDTASLTITGAQAEDEGDYYCNSRDRSGTVVFGGGTKLTV | Binder 2 |
| | LGGGGSGGGGSGGGGSEVQLVESGGGVVQPGRSLRLSCAASGFTFSSYAMSW | |
| | VRQAPGKGLEWVSAISGSGGSTYYAGSVKGRFTISRDNSKNTLYLQMNSLRA | |
| | EDTAVYYCARDLYGYRYYYYGMDVWGQGTMVTVSS | |
| 530 | SSELTQDPAVSVALGQTVRITCQGDSLRSYYANWYQQKPGQAPILVNYGKNNR | GPRC5D |
| | PSGIPDRFSGSSSGKTASLTITGAQAEDEADYYCSSRDSSGNHLVVFGGGTQLT | Binder 3 |
| | VLGGGGSGGGGSGGGGSQVQLQQSGPGQVKPSQTLSLTCAISGDSVSSNSAA | |
| | WNWIRQSPSRGLEWLGRTYYRSKWYNDYAVSVKSRITINPDTSKNQFSLQLNS | |
| | VTPEDTAVYYCARAYSPSRLRWSRAAAFDIWGQGTTVTVSS | |
| 531 | ESKYGPPCPPCP | IgG4 Hinge |
| 532 | TTTPAPRPPTPAPTIASQPLSLRPE | CD8 Hinge |
| 533 | IEVMYPPPYLDNEKSNGTIIHVKGKHLCPSPLFPGPSKP | CD28 Hinge |
| 534 | ACRPAAGGAVHTRGLDFACDIYIWAPLAGTCGVLLLSLVITLYC | CD8 TM |
| 535 | FWVLVVVGGVLACYSLLVTVAFIIFWV | CD28 TM |
| 536 | MFWVLVVVGGVLACYSLLVTVAFIIFWV | CD28 TM |
| 537 | RSKRSRLLHSDYMNMTPRRPGPTRKHYQPYAPPRDFAAYRS | CD28 Costim |
| 538 | KRGRKKLLYIFKQPFMRPVQTTQEEDGCSCRFPEEEEGGCEL | 4-1BB Costim |
| 539 | RVKFSRSADAPAYQQGQNQLYNELNLGRREEYDVLDKRRGRDPEMGGKPRR | CD3zeta |
| | KNPQEGLYNELQKDKMAEAYSEIGMKGERRRGKGHDGLYQGLSTATKDTYD | |
| | ALHMQALPPR | |
| 540 | RVKFSRSADAPAYKQGQNQLYNELNLGRREEYDVLDKRRGRDPEMGGKPRR | CD3zeta |
| | KNPQEGLYNELQKDKMAEAYSEIGMKGERRRGKGHDGLYQGLSTATKDTYD | (Q > K) |
| | ALHMQALPPR | |
| 541 | MWPLVAALLLGSACCGSAQLLFNKTKSVEFTFCNDTVVIPCFVTNMEAQNTT | Amino acid |
| | EVYVKWKFKGRDIYTFDGALNKSTVPTDFSSAKIEVSQLLKGDASLKMDKSD | sequence |
| | AVSHTGNYTCEVTELTREGETIIELKYRVVSWFSPNENILIVIFPIFAILLFWGQF | encoded by |
| | GIKTLKYRSGGMDEKTIALLVAGLVITVIVIVGAILFVPGEYSLKNATGLGLIVT | CDS of |
| | STGILILLHYYVFSTAIGLTSFVIAILVIQVIAYILAVVGLSLCIAACIPMHGPLLIS | NM_001777.4 |
| | GLSILALAQLLGLVYMKFVASNQKTIQPPRKAVEEPLNAFKESKGMMNDE | |
| 542 | MWPLVAALLLGSACCGSAQLLENKTKSVEFTFCNDTVVIPCFVTNMEAQNTT | Amino acid |
| | EVYVKWKFKGRDIYTFDGALNKSTVPTDESSAKIEVSQLLKGDASLKMDKSD | sequence |
| | AVSHTGNYTCEVTELTREGETIIELKYRVVSWFSPNENILIVIFPIFAILLFWGQF | encoded by |
| | GIKTLKYRSGGMDEKTIALLVAGLVITVIVIVGAILFVPGEYSLKNATGLGLIVT | CDS of |
| | STGILILLHYYVFSTAIGLTSFVIAILVIQVIAYILAVVGLSLCIAACIPMHGPLLIS | NM_198793.2 |
| | GLSILALAQLLGLVYMKFVASNQKTIQPPRNN | |
| 543 | atgtggcccctggtagcggcgctgttgctgggctcggcgtgctgcggatcagctcagctactatttaat | Nucleotide |
| | aaaacaaaatctgtagaattcacgttttgtaatgacactgtcgtcattccatgctttgttactaatatggag | sequence of |
| | gcacaaaacactactgaagtatacgtaaagtggaaatttaaaggaagagatatttacacctttgatgga | NM_001777.4 |
| | gctctaaacaagtccactgtccccactgactttagtagtgcaaaaattgaagtctcacaattactaaaag | CDS (nts 124- |
| | gagatgcctctttgaagatggataagagtgatgctgtctcacacacaggaaactacacttgtgaagtaa | 1095) |
| | cagaattaaccagagaaggigaaacgatcalcgagctaaaatatcgtgtigtttcalggitttctccaaat | |
| | gaaaatattcttattgttattttcccaatttttgctatactcctgttctggggacagtttggtattaaaacactta | |
| | aatatagatccggtggtatggatgagaaaacaattgctttacttgttgctggactagtgatcactgtcatt | |
| | gtcattgttggagccattcttttcgtcccaggtgaatattcattaaagaatgctactggccttggtttaattgt | |
| | gacttctacagggatattaatattacttcactactatgtgtttagtacagcgattggattaacctccttcgtc | |
| | attgccatattggttattcaggtgatagcctatatcctcgctgtggttggactgagtctctgtattgcggcg | |
| | tgtataccaatgcatggccctcttctgatttcaggtttgagtatcttagctctagcacaattacttggactag | |
| | tttatatgaaatttgtggcttccaatcagaagactatacaacctcctaggaaagctgtagaggaacccctt | |
| | aatgcattcaaagaatcaaaaggaatgatgaatgatgaataa | |
| 544 | atgtggcccctggtagcggcgctgttgctgggctcggcgtgctgcggatcagctcagctactatttaataaaacaaa | Nucleotide |
| | atctgtagaattcacgttttgtaatgacactgtcgtcattccatgctttgttactaatatggaggcacaaaacacta | sequence of |
| | ctgaagtatacgtaaagtggaaatttaaaggaagagatatttacacctttgatggagctctaaacaagtccactgtc | NM_198793.2 |
| | cccactgactttagtagtgcaaaaattgaagtctcacaattactaaaaggagatgcctctttgaagatggataagag | CDS (nts 181- |
| | tgatgctgtctcacacacaggaaactacacttgtgaagtaacagaattaaccagagaaggtgaaacgatcatcgagc | 1098) |
| | taaaatatcgtgttgtttcatggttttctccaaatgaaaatattcttattgttattttcccaatttttgctatactc | |
| | ctgttctggggacagtttggtattaaaacacttaaatatagatccggtggtatggatgagaaaacaattgctttact | |
| | tgttgctggactagtgatcactgtcattgtcattgttggagccattcttttcgtcccaggtgaatattcattaaaga | |
| | atgctactggccttggtttaattgtgacttctacagggatattaatattacttcactactatgtgtttagtacagcg | |
| | attggattaacctccttcgtcattgccatattggttattcaggtgatagcctatatcctcgctgtggttggactgag | |
| | tctctgtattgcggcgtgtataccaatgcatggccctcttctgatttcaggtttgagtatcttagctctagcacaat | |
| | tacttggactagtttatatgaaatttgtggcttccaatcagaagactatacaacctcctaggaataactga | |
| 545 | atgtggcccctggtcgccgccctgttgctgggctcggcatgctgcggatcagctcagctactgtttaataaaacaaa | Codon- |
| | atctgtagaattcacgttttgtaacgacactgtcgtgatcccatgctttgttactaatatggaggcacaaaacacca | optimized |
| | ctgaagtgtacgtgaagtggaaattcaaaggcagagacatttacacctttgacggcgccctcaacaagtccaccgtg | nucleotide |
| | cccactgactttagtagcgcaaaaattgaggtcagccaattactaaaaggagatgcctctttgaagatggacaagag | sequence |
| | cgatgctgtcagccacacagggaactacacttgtgaagtaacagagttaacccgcgaaggtgaaacgatcatcgagc | encoding CD47 |
| | tgaagtatcgagtggtgtcctggttttctccgaacgagaatatccttatcgtaattttcccaattttcgctatcctc | |
| | ctgttctggggccagtttggtatcaagacactcaaatatcggtccggtgggatggatgagaagacaattgccctgct | |
| | tgttgctggactcgtgatcaccgtcatcgtgattgttggggccatccttttcgtcccaggggagtacagcctgaaga | |
| | atgctacgggcctgggattaattgtgacctctacagggatactcatcctgcttcactactatgtgttcagtaccgcg | |
| | attggactgacctccttcgtcattgccatattggtgattcaggtgatagcctacatcctcgccgtggttggcctgag | |
| | tctctgtatcgcggcgtgcatacccatgcatggccctcttctgatttcagggttgagtatcctcgcactagcacagt | |
| | tgctgggactggtttatatgaaatttgtggcctccaaccagaagactatacagcctcctaggaaggctgtagaggag | |
| | cccctgaatgcattcaaggaatcaaaaggcatgatgaatgatgaa | |
| 546 | (GSG)EGRGSLLTCGDVEENPGP | T2A |
| 547 | (GSG)ATNFSLLKQAGDVEENPGP | P2A |
| 548 | (GSG)QCTNYALLKLAGDVESNPGP | E2A |
| 549 | (GSG)VKQTLNFDLLKLAGDVESNPGP | F2A |
| 550 | RRRR(GSG) | FC1 |
| 551 | RKRR(GSG) | FC2 |
| 552 | RKRR(GSG)TPDPW(GSG) | FC3 |
| 553 | SYWIE | CD4 Binder 1- |
| | | HCDR1 |
| 554 | DYVIS | CD4 Binder 2- |
| | | HCDR1 |
| 555 | THWMH | CD4 Binder 3- |
| | | HCDR1 |
| 556 | GYWMY | CD4 Binder 4- |
| | | HCDR1 |
| 557 | EILPGSGSTSYNEKFKG | CD4 Binder 1- |
| | | HCDR2 |
| 558 | EIYPGSGSSYYNEKFKG | CD4 Binder 2- |
| | | HCDR2 |
| 559 | MINPSDGVTYYAQTFQG | CD4 Binder 3- |
| | | HCDR2 |
| 560 | AISPGGGSTYYPDSVKG | CD4 Binder 4- |
| | | HCDR2 |
| 561 | RGYGYDEGFDY | CD4 Binder 1- |
| | | HCDR3 |
| 562 | PGDLGFAY | CD4 Binder 2- |
| | | HCDR3 |
| 563 | EYYGEGFDY | CD4 Binder 3- |
| | | HCDR3 |
| 564 | SLTATHTYEYDY | CD4 Binder 4- |
| | | HCDR3 |
| 565 | ASQDINSYLS | CD4 Binder 1- |
| | | LCDR1 |
| 566 | ASQSVDYDGDSYMN | CD4 Binder 2 |
| | | LCDR1 |
| 567 | RASQGISNYLA | CD4 Binder 3- |
| | | LCDR1 |
| 568 | RANRLVD | CD4 Binder 1- |
| | | LCDR2 |
| 569 | AASNLES | CD4 Binder 2- |
| | | LCDR2 |
| 570 | SASNLQS | CD4 Binder 3 |
| | | LCDR2 |
| 571 | LQYDEFPPT | CD4 Binder 1- |
| | | LCDR3 |
| 572 | QQSNKDPFT | CD4 Binder 2- |
| | | LCDR3 |
| 573 | QQSYSTPLT | CD4 Binder 3- |
| | | LCDR3 |
| 574 | QVQLQQSGAELMKPGASVKISCKATGYTFSSYWIEWVKQRPGHGLEWIGEILP | CD4 Binder 1- |
| | GSGSTSYNEKFKGKATFTADTSSNTAYMQLSSLTSEDSAVYYCARRGYGYDEG | VH |
| | FDYWGQGTTLTVSS | |
| 575 | QVQLQQSGPELVKPGASVKMSCKASGYTFTDYVISWVRQAPGQGLEWIGEIY | CD4 Binder 2- |
| | PGSGSSYYNEKFKGRATLTADKSSNTAYMQLSSLRSEDSAVYFCARPGDLGFA | VH |
| | YWGQGTLVTVSS | |
| 576 | QVQLVQSGAEVKKPGASVKVSCKASGYSLITHWMHWVRQAPGQGLEWMG | CD4 Binder 3- |
| | MINPSDGVTYYAQTFQGRVTMTRDTSTSTVYMELSSLRSEDTAVYYCAREYY | VH |
| | GEGFDYWGQGTLVTVSS | |
| 577 | EVQLVESGGGLVQSGGSLRLSCAASGFTFSGYWMYWVRQAPGKGLEWVSAI | CD4 Binder 4 |
| | SPGGGSTYYPDSVKGRFTISRDNAKNTLYLQMNSLEPEDTALYYCASSLTATHT | VH |
| | YEYDYWGQGTQVTVSS | |
| 578 | DIKMTQSPSSMYASLGERVTITCKASQDINSYLSWFQQKPGKSPKTLIYRANRL | CD4 Binder 1- |
| | VDGVPSRFSGSGSGQDYSLTISSLEYEDMGIYYCLQYDEFPPTFGAGTKLELKR | VL |
| 579 | DIVLTQSPSSLAVSLGQRATISCKASQSVDYDGDSYMNWYQQKPGQPPKLLIY | CD4 Binder 2- |
| | AASNLESGIPARFSGSGSGTDFTLTIHPVEEEDAATYYCQQSNKDPFTFGGGTK | VL |
| | LELKR | |
| 580 | DIQMTQSPSSLSASVGDRVTITCRASQGISNYLAWYQQKPGKAPKLLIYSASNL | CD4 Binder 3- |
| | QSGVPSRFSGSGSGTDFTLTISSLQPEDFATYYCQQSYSTPLTFGGGTKVEIKR* | VL |
| 581 | SYAIS | CD8 Binder 1- |
| | | HCDR1 |
| 582 | DYYIQ | CD8 Binder 2- |
| | | HCDR1 |
| 583 | SYYMH | CD8 Binder 3- |
| | | HCDR1 |
| 584 | NHYMH | CD8 Binder 4- |
| | | HCDR1 |
| 585 | IIDPSDGNTNYAQNFQG | CD8 Binder 1- |
| | | HCDR2 |
| 586 | WINPNSGGTSYAQKFQG | CD8 Binder 2- |
| | | HCDR2 |
| 587 | GFDPEDGETIYAQKFQG | CD8 Binder 3 |
| | | HCDR2 |
| 588 | WMNPNSGNTGYAQKFQG | CD8 Binder 4- |
| | | HCDR2 |
| 589 | ERAAAGYYYYMDV | CD8 Binder 1- |
| | | HCDR3 |
| 590 | EGDYYYGMDA | CD8 Binder 2- |
| | | HCDR3 |
| 591 | DQGWGMDV | CD8 Binder 3- |
| | | HCDR3 |
| 592 | SESGSDLDY | CD8 Binder 4- |
| | | HCDR3 |
| 593 | RASQSISSYLN | CD8 Binder 1- |
| | | LCDR1 |
| 594 | RSSQSLLHSNGYNYLD | CD8 Binder 2- |
| | | LCDR1 |
| 595 | RASQSISSYLN | CD8 Binder 3- |
| | | LCDR1 |
| 596 | RASQTIGNYVN | CD8 Binder 4- |
| | | LCDR1 |
| 597 | AASSLQS | CD8 Binder 1- |
| | | LCDR2 |
| 598 | LGSNRAS | CD8 Binder 2 |
| | | LCDR2 |
| 599 | AASSLQS | CD8 Binder 3- |
| | | LCDR2 |
| 600 | GASNLHT | CD8 Binder 4- |
| | | LCDR2 |
| 601 | QQSYSTPLT | CD8 Binder 1- |
| | | LCDR3 |
| 602 | MQGLQTPHT | CD8 Binder 2 |
| | | LCDR3 |
| 603 | QQTYSTPYT | CD8 Binder 3 |
| | | LCDR3 |
| 604 | QQTYSAPLT | CD8 Binder 4- |
| | | LCDR3 |
| 605 | QVQLVQSGAEVKKPGASVKVSCKASGGTFSSYAISWVRQAPGQGLEWMGIID | CD8 Binder 1- |
| | PSDGNTNYAQNFQGRVTMTRDTSTSTVYMELSSLRSEDTAVYYCAKERAAAG | VH |
| | YYYYMDVWGQGTTVTVSS | |
| 606 | QVQLVQSGAEVKKPGASVKVSCKASGYTFTDYYIQWVRQAPGQGLEWMGW | CD8 Binder 2- |
| | INPNSGGTSYAQKFQGRVTMTRDTSTSTVYMELSSLRSEDTAVYYCAKEGDY | VH |
| | YYGMDAWGQGTMVTVSS | |
| 607 | QVQLVQSGAEVKKPGASVKVSCKASGYTFTSYYMHWVRQAPGQGLEWMGG | CD8 Binder 3- |
| | FDPEDGETIYAQKFQGRVTMTRDTSTSTVYMELSSLRSEDTAVYYCARDQGW | VH |
| | GMDVWGQGTTVTVSS | |
| 608 | QVQLVQSGAEVKKPGASVKVSCKASGYTFTNHYMHWVRQAPGQGLEWMG | CD8 Binder 4- |
| | WMNPNSGNTGYAQKFQGRVTMTRDTSTSTVYMELSSLRSEDTAVYYCASSES | VH |
| | GSDLDYWGQGTLVTVSS | |
| 609 | DIQMTQSPSSLSASVGDRVTITCRASQSISSYLNWYQQKPGKAPKLLIYAASSL | CD8 Binder 1- |
| | QSGVPSRFSGSGSGTDFTLTISSLQPEDFATYYCQQSYSTPLTFGGGTKVEIKR | VL |
| 610 | DIVMTQSPLSLPVTPGEPASISCRSSQSLLHSNGYNYLDWYLQKPGQSPQLLIY | CD8 Binder 2- |
| | LGSNRASGVPDRFSGSGSGTDFTLKISRVEAEDVGVYYCMQGLQTPHTFGQG | VL |
| | TKVEIKR | |
| 611 | DIQMTQSPSSLSASVGDRVTITCRASQSISSYLNWYQQKPGKAPKLLIYAASSL | CD8 Binder 3- |
| | QSGVPSRFSGSGSGTDFTLTISSLQPEDFATYYCQQTYSTPYTFGQGTKLEIKR | VL |
| 612 | DIQMTQSPSSLSASVGDRVTITCRASQTIGNYVNWYQQKPGKAPKLLIYGASN | CD8 Binder 4- |
| | LHTGVPSRFSGSGSGTDFTLTISSLQPEDFATYYCQQTYSAPLTFGGGTKVEIKR | VL |

### EQUIVALENTS

It is to be understood that while the disclosure has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An engineered lentiviral vector comprising:
a. a first genome comprising a first transgene; and
b. a second genome comprising a second transgene,
wherein the first transgene encodes a first chimeric antigen receptor (CAR) and the second transgene encodes a second CAR, wherein the first CAR and the second CAR comprise different antigen binding domains that target different antigens expressed on a target cell.

2. The engineered lentiviral vector of claim 1, wherein:
a. the first CAR is a CD19 CAR and the second CAR is a CD22 CAR;
b. the first CAR is a B cell maturation agent (BCMA) CAR and the second CAR is a G-protein coupled receptor family C group 5 member D (GPRC5D) CAR;
c. the first CAR is a CD19 CAR and the second CAR is a CD20 CAR;
d. the first CAR is a CD20 CAR and the second CAR is a CD22 CAR; or
d. the first CAR is a CD19 CAR and the second CAR is a BCMA CAR.

3. The engineered lentiviral vector of claim 1, wherein the first CAR and the second CAR are selected from the group comprising or consisting of a CD19 CAR, a CD22 CAR, B cell maturation agent (BCMA) CAR, a G-protein coupled receptor family C group 5 member D (GPRC5D) CAR, a CD5 CAR, a CD20 CAR, a CD23 CAR, a CD30 CAR, a CD33 CAR, CD38 CAR, a CD70 CAR, a CD123 CAR, a CD138 CAR, a Kappa CAR, Lambda CAR, a CD123 CAR, a LeY CAR, a NKG2D ligand CAR, a WT1 CAR, a GD2 CAR, a HER2 CAR, an EGFR CAR, an EGFRvIII CAR, a B7H3 CAR, a PSMA CAR, a PSCA CAR, a CAIX CAR, a CD171 CAR, a CEA CAR, a CSPG4 CAR, a EPHA2 CAR, a FAP CAR, a FRα CAR, a IL-13Rα CAR, a Mesothelin CAR, a MUC1 CAR, a MUC16 CAR, a ROR1 CAR, a C-Met CAR, a CD133 CAR, a Ep-CAM CAR, a GPC3 CAR, a HPV16-E6 CAR, a IL13Ra2 CAR, a MAGEA3 CAR, a MAGEA4 CAR, a MART1 CAR, a NY-ESO-1 CAR, a VEGFR2 CAR, a α-Folate receptor CAR, a CD24 CAR, a CD44v7/8 CAR, a EGP-2 CAR, a EGP-40 CAR, a erb-B2 CAR, a erb-B 2,3,4 CAR, a FBP CAR, a Fetal acethylcholine e receptor CAR, a GD2 CAR, a GD3 CAR, a HMW-MAA CAR, a IL-11Rα CAR, a KDR CAR, a Lewis Y CAR, a L1-cell adhesion molecule CAR, a MAGE-A1 CAR, a Oncofetal antigen (h5T4) CAR, a TAG-72 CAR, or a CD19/CD22 bispecific CAR.

4. The engineered lentiviral vector of claim 1, the first CAR and the second CAR are selected from the group comprising or consisting of a CD19 CAR, a CD22 CAR, BCMA CAR, a GPRC5D CAR, a CD20 CAR, a CD19/CD22-bispecific CAR, a CD38 CAR, a CD123 CAR, or a CD138 CAR.

5. The engineered lentiviral vector of any one of claims 1-4, wherein the first genome and/or the second genome further comprises a transgene encoding a tolerogenic factor.

6. The engineered lentiviral vector of any one of claims 1-5, wherein the first transgene comprises a biscistronic or multicistronic expression cassette encoding the first CAR and a tolerogenic factor and/or the second transgene comprises a biscistronic or multicistronic expression cassette encoding the second CAR and a tolerogenic factor.

7. The engineered lentiviral vector of claim 5 or 6, wherein the tolerogenic factor is selected from the group comprising or consisting of CD47, a SIRPα engager, A20/TNFAIP3, B2M-HLA-E, CD16, CD16 Fc receptor, CD24, CD27, CD35, CD39, CD46, CD47, CD52, CD55, CD59, CD64, CD200, CCL21, CCL22, CTLA4-Ig, C1 inhibitor, CR1, DUX4, FASL, HLA-C, HLA-E, HLA-E heavy chain, HLA-F, HLA-G, H2-M3, IDO1, IL-10, IL15-RF, IL-35, IL-39, MANF, Mfge8, PD-L1, or Serpinb9.

8. The engineered lentiviral vector of claim 5 or 6, wherein the tolerogenic factor is CD47.

9. The engineered lentiviral vector of claim 5 or 6, wherein the first genome comprises a first tolerogenic factor and the second genome comprises a second tolerogenic factor, and the first and second tolerogenic factors are different.

10. The engineered lentiviral vector of claim 9, wherein the first tolerogenic factor and the second tolerogenic factor are selected from the group comprising or consisting of CD47, a SIRPα engager, A20/TNFAIP3, B2M-HLA-E, CD16, CD16 Fe receptor, CD24, CD27, CD35, CD39, CD46, CD47, CD52, CD55, CD59, CD64, CD200, CCL21, CCL22, CTLA4-Ig, C1 inhibitor, CR1, DUX4, FASL, HLA-C, HLA-E, HLA-E heavy chain, HLA-F, HLA-G, H2-M3, IDO1, IL-10, IL15-RF, IL-35, IL-39, MANF, Mfge8, PD-L1, or Serpinb9.

11. The engineered lentiviral vector of claim 5 or 6, wherein the first genome comprises a tolerogenic factor and the second genome does not comprise a tolerogenic factor.

12. An engineered lentiviral vector comprising a first genome and a second genome, wherein the first genome comprises a first transgene and the second genome comprises a second transgene, and wherein the first and the second transgene are different.

13. The engineered lentiviral vector of claim 12, wherein the first transgene encodes a sequence-specific nuclease.

14. The engineered lentiviral vector of claim 13, wherein the sequence-specific nuclease is an RNA-guided nuclease, a transcription activator-like effector nuclease (TALEN), a meganuclease, a CRISPR-associated transposase, and a TnpB polypeptide, or a zinc-finger nuclease (ZFN).

15. The engineered lentiviral vector of claim 13 or 14, wherein the sequence-specific nuclease is selected from the group consisting of: Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, a CRISPR-associated transposase, and a TnpB polypeptide.

16. The engineered lentiviral vector of claim 14, wherein the RNA-guided nuclease comprises a Cas nuclease.

17. The engineered lentiviral vector of claim 16, wherein the Cas nuclease is a Type 11 or a Type V Cas protein.

18. The engineered lentiviral vector of claim 16 or 17, wherein the Cas nuclease is selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, and Mad7.

19. The engineered lentiviral vector of claim 16 or 17, wherein the Cas nuclease a Cas9 or a Cas12b.

20. The engineered lentiviral vector of any one of claims 16-19, wherein the RNA-guided nuclease comprises a Cas nuclease and a guide RNA (CRISPR-Cas combination).

21. The engineered lentiviral vector of claim 12, wherein the first transgene encodes a gRNA.

22. The engineered lentiviral vector of claim 12, wherein the first transgene encodes an antibody or portion thereof.

23. The engineered lentiviral vector of claim 12, wherein the first transgene encodes an antigen.

24. The engineered lentiviral vector of claim 12, wherein the first transgene encodes a therapeutic polypeptide.

25. The engineered lentiviral vector of claim 24, wherein the therapeutic polypeptide is useful for protein replacement therapy.

26. The engineered lentiviral vector of claim 12, wherein the first transgene encodes a CAR.

27. The engineered lentiviral vector any one of claims 12-26, wherein the second transgene encodes a sequence-specific nuclease.

28. The engineered lentiviral vector of claim 27, wherein the sequence-specific nuclease is an RNA-guided nuclease, a transcription activator-like effector nuclease (TALEN), a meganuclease, a CRISPR-associated transposase, and a TnpB polypeptide, or a zinc-finger nuclease (ZFN).

29. The engineered lentiviral vector of claim 27 or 28, wherein the sequence-specific nuclease is selected from the group consisting of: Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, a CRISPR-associated transposase, and a TnpB polypeptide.

30. The engineered lentiviral vector of claim 28, wherein the RNA-guided nuclease comprises a Cas nuclease.

31. The engineered lentiviral vector of claim 30, wherein the Cas nuclease is a Type II or Type V Cas protein.

32. The engineered lentiviral vector of claim 30 or 31, wherein the Cas nuclease is selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, and Mad7.

33. The engineered lentiviral vector of claim 31 or 32, wherein the Cas nuclease a Cas9 or a Cas12b.

34. The engineered lentiviral vector of any one of claims 28-33, wherein the RNA-guided nuclease comprises a Cas nuclease and a guide RNA (CRISPR-Cas combination).

35. The engineered lentiviral vector of any one of claims 12-26, wherein the second transgene encodes a gRNA.

36. The engineered lentiviral vector of any one of claims 12-26, wherein the second transgene encodes an antibody or portion thereof.

37. The engineered lentiviral vector of any one of claims 12-26, wherein the second transgene encodes an antigen.

38. The engineered lentiviral vector of any one of claims 12-26, wherein the second transgene encodes a therapeutic polypeptide.

39. The engineered lentiviral vector of claim 38, wherein the therapeutic polypeptide is useful for protein replacement therapy.

40. The engineered lentiviral vector of any one of any one of claims 12-26, wherein the second transgene encodes a CAR.

41. The engineered lentiviral vector of any one of any one of claims 12-40, wherein the CAR is selected from the group comprising or consisting of: a CD19 CAR, a CD22 CAR, B cell maturation agent (BCMA) CAR, a G-protein coupled receptor family C group 5 member D (GPRC5D) CAR, a CD5 CAR, a CD20 CAR, a CD23 CAR, a CD30 CAR, a CD33 CAR, CD38 CAR, a CD70 CAR, a CD123 CAR, a CD138 CAR, a Kappa CAR, Lambda CAR, a CD123 CAR, a LeY CAR, a NKG2D ligand CAR, a WT1 CAR, a GD2 CAR, a HER2 CAR, an EGFR CAR, an EGFRvIII CAR, a B7H3 CAR, a PSMA CAR, a PSCA CAR, a CAIX CAR, a CD171 CAR, a CEA CAR, a CSPG4 CAR, a EPHA2 CAR, a FAP CAR, a FRα CAR, a IL-13Rα CAR, a Mesothelin CAR, a MUC1 CAR, a MUC16 CAR, a ROR1 CAR, a C-Met CAR, a CD133 CAR, a Ep-CAM CAR, a GPC3 CAR, a HPV16-E6 CAR, a IL13Ra2 CAR, a MAGEA3 CAR, a MAGEA4 CAR, a MART1 CAR, a NY-ESO-1 CAR, a VEGFR2 CAR, a α-Folate receptor CAR, a CD24 CAR, a CD44v7/8 CAR, a EGP-2 CAR, a EGP-40 CAR, a erb-B2 CAR, a erb-B 2,3,4 CAR, a FBP CAR, a Fetal acethylcholine e receptor CAR, a GD2 CAR, a GD3 CAR, a HMW-MAA CAR, a IL-11Rα CAR, a KDR CAR, a Lewis Y CAR, a L1-cell adhesion molecule CAR, a MAGE-A1 CAR, a Oncofetal antigen (h5T4) CAR, a TAG-72 CAR, or a CD19/CD22 bispecific CAR.

42. The engineered lentiviral vector of claim 41, wherein the first transgene encodes a first CAR and the second transgene encodes a second CAR, and the first CAR and the second CAR are selected from the group comprising or consisting of a CD19 CAR, a CD22 CAR, BCMA CAR, a GPRC5D CAR, a CD20 CAR, a CD19/CD22-bispecific CAR, a CD38 CAR, a CD123 CAR, or a CD138 CAR.

43. The engineered lentiviral vector of claim 42, wherein:
a. the first CAR is a CD19 CAR and the second CAR is a CD22 CAR,
b. the first CAR is a BCMA CAR and the second CAR is a GPRC5D CAR;
c. the first CAR is a CD19 CAR and the second CAR is a CD20 CAR;
d. the first CAR is a CD20 CAR and the second CAR is a CD22 CAR; or
d. the first CAR is a CD19 CAR and the second CAR is a BCMA CAR.

44. The engineered lentiviral vector of any one of claims 12-43, wherein the first genome and/or the second genome further comprises a transgene encoding a tolerogenic factor.

45. The engineered lentiviral vector of any one of claims 12-44, wherein the first transgene and/or the second transgene comprises a biscistronic or a multicistronic expression cassette.

46. The engineered lentiviral vector of claim 44-45, wherein the tolerogenic factor is selected from the group comprising or consisting of CD47, a SIRPα engager, A20/TNFAIP3, B2M-HLA-E, CD16, CD16 Fc receptor, CD24, CD27, CD35, CD39, CD46, CD47, CD52, CD55, CD59, CD64, CD200, CCL21, CCL22, CTLA4-Ig, C1 inhibitor, CR1, DUX4, FASL, HLA-C, HLA-E, HLA-E heavy chain, HLA-F, HLA-G, H2-M3, IDO1, IL-10, IL15-RF, IL-35, IL-39, MANF, Mfge8, PD-L1, or Serpinb9.

47. The engineered lentiviral vector of any one of claims 44-46, wherein the tolerogenic factor is CD47.

48. The engineered lentiviral vector of any one of claims 44-46, wherein the first genome comprises a first tolerogenic factor and the second genome comprises a second tolerogenic factor, and the first and the second tolerogenic factors are different.

49. The engineered lentiviral vector of claim 48, wherein the first tolerogenic factor and the second tolerogenic factor are selected from the group comprising or consisting of CD47, a SIRPα engager, A20/TNFAIP3, B2M-HLA-E, CD16, CD16 Fe receptor, CD24, CD27, CD35, CD39, CD46, CD47, CD52, CD55, CD59, CD64, CD200, CCL21, CCL22, CTLA4-Ig, C1 inhibitor, CR1, DUX4, FASL, HLA-C, HLA-E, HLA-E heavy chain, HLA-F, HLA-G, H2-M3, IDO1, IL-10, IL15-RF, IL-35, IL-39, MANF, Mfge8, PD-L1, or Serpinb9.

50. The engineered lentiviral vector of any one of claims 44-47, wherein first genome comprises a tolerogenic factor and the second genome does not comprise a tolerogenic factor.

51. The engineered lentiviral vector any one of claims 12-43, wherein the first genome further comprises a transgene encoding CD47 and the second genome further comprises a transgene encoding CD47.

52. The engineered lentiviral vector of any one of claims 1-51, further comprising a lipid bilayer envelope that comprises one or more fusogens.

53. The engineered lentiviral vector of claim 52, wherein the one or more fusogens comprise at least one fusogen that has a tropism for B cells, T cells, natural killer cells, islet cells, glial progenitor cells, neuronal cells, hematopoietic stem cells, cardiac cells, hepatocytes, stem cells, or induced pluripotent stem cells.

54. The engineered lentiviral vector of any one of claims 52-53, wherein the one or more fusogens comprise at least one fusogen that has an endogenous tropism.

55. The engineered lentiviral vector of any one of claims 52-54, wherein the one or more fusogens comprise at least one fusogen that has engineered tropism.

56. The engineered lentiviral vector of any one of claims 52-55, wherein the one or more fusogens comprise one or more viral fusogens.

57. The engineered lentiviral vector of any one of claims 52-56, wherein the one or more fusogens comprise at least one fusogen that is involved in attachment of a viral vector to a cell membrane.

58. The engineered lentiviral vector of any one of claims 52-57, wherein the one or more fusogens comprise at least one fusogen that is involved in directing fusion of the lipid bilayer of a viral vector and a cell membrane.

59. The engineered lentiviral vector of any one of claims 52-58, wherein the one or more fusogens comprise one or more paramyxovirus envelope proteins or biologically active portions thereof.

60. The engineered lentiviral vector of claim 59, wherein the one or more paramyxovirus envelope proteins or biologically active portions thereof comprises a paramyxovirus glycoprotein (Protein G) or biologically active portion thereof.

61. The engineered lentiviral vector of claim 59 or 60, wherein the one or more paramyxovirus envelope proteins or biologically active portions thereof comprises a paramyxovirus fusion protein (Protein F) or biologically active portion thereof.

62. The engineered lentiviral vector of any one of claims 52-61, wherein the one or more fusogens comprise a Nipah virus fusion protein or a functional variant thereof.

63. The engineered lentiviral vector of any one of claims 52-62, wherein the one or more fusogens comprise a Nipah virus F glycoprotein (NiV-F) or a biologically active portion thereof and a Nipah virus G glycoprotein (NiV-G) or a biologically active portion thereof.

64. The engineered lentiviral vector any one of claims 52-63, wherein the one or more fusogens comprise one or more chimeric proteins.

65. The engineered lentiviral vector of claim 64, wherein the one or more chimeric proteins comprise at least one chimeric protein that comprises a paramyxovirus envelope protein or biologically active portion thereof.

66. The engineered lentiviral vector of claim 64 or 65, wherein the one or more chimeric proteins comprise at least one chimeric protein that comprises a targeting agent that targets a target molecule.

67. The engineered lentiviral vector of any one of claims 64-66, wherein the one or more chimeric proteins comprise at least one chimeric protein that comprises (i) a paramyxovirus envelope protein or biologically active portion thereof and (ii) an targeting agent.

68. The engineered lentiviral vector of any one of claims 63-67, wherein the NiV-G protein or biologically active portion thereof is fused to a targeting agent.

69. The engineered lentiviral vector of claim 68, wherein the targeting agent is fused to the C-terminus of the NiV-G protein or biologically active portion thereof.

70. The engineered lentiviral vector of any of claims 63-69, wherein the NiV-G or biologically active portion thereof is a wild-type NiV-G protein or a functionally active variant or biologically active portion thereof.

71. The engineered lentiviral vector of any of claims 63-69, wherein the NiV-G protein or biologically active portion thereof is truncated and lacks up to 40 contiguous amino acid residues at or near the N-terminus of the wild-type NiV-G protein set forth in SEQ ID NO:4.

72. The engineered lentiviral vector of any of claims 63-69 and 71, wherein the NiV-G protein or biologically active portion thereof is a biologically active portion that is a truncated NiV-G that has a deletion of amino acids 2-34 at or near the N-terminus of wild-type NiV-G set forth in SEQ ID NO:4.

73. The engineered lentiviral vector of any of claims 63-72, wherein the NiV-G protein or biologically active portion thereof does not include an initial methionine.

74. The engineered lentiviral vector of any of claims 63-73, wherein the NiV-G protein or biologically active portion thereof has the amino acid sequence set forth in SEQ ID NO:42 or a sequence of amino acids that exhibits at least at or about 80%, 85%, 90%, or 95% sequence identity to the sequence set forth in SEQ ID NO:42.

75. The engineered lentiviral vector of any of claims 63-74, wherein the NiV-G protein or biologically active portion thereof is set forth in SEQ ID NO:42.

76. The engineered lentiviral vector of any of claims 52-75, wherein at least one of the one or more fusogens comprise one or more modifications to reduce binding to its native receptor.

77. The engineered lentiviral vector of claim 76, wherein the NiV-G protein or biologically active portion thereof is a mutant NiV-G protein or biologically active portion thereof that exhibits reduced binding to Ephrin B2 or Ephrin B3.

78. The engineered lentiviral vector of claim 76 or 77, wherein the NiV-G protein or biologically active portion thereof comprises one or more amino acid substitutions corresponding to amino acid substitutions selected from the group consisting of E501A, W504A, Q530A, and E533A with reference to numbering set forth in SEQ ID NO:4.

79. The engineered lentiviral vector of any of claims 76-78, wherein the NiV-G protein or biologically active portion thereof has the amino acid sequence set forth in SEQ ID NO: 17 or a sequence of amino acids that exhibits at least at or about 80%, 85%, 90%, or 95% sequence identity to the sequence set forth in SEQ ID NO:17.

80. The engineered lentiviral vector of any of claims 76-78, wherein the NiV-G protein or biologically active portion thereof is set forth in SEQ ID NO:17.

81. The engineered lentiviral vector of any of claims 76-78, wherein the NiV-G protein or the biologically active portion has the amino acid sequence set forth in SEQ ID NO: 18 or a sequence of amino acids that exhibits at least at or about 80%, 85%, 90%, or 95% sequence identity to the sequence set forth in SEQ ID NO: 18.

82. The engineered lentiviral vector of any of claims 76-78, wherein the NiV-G protein or biologically active portion thereof is set forth in SEQ ID NO:18.

83. The engineered lentiviral vector of any of claims 63-82, wherein the NiV-F protein or the biologically active portion thereof is a wild-type NiV-F protein or a functionally active variant or biologically active portion thereof.

84. The engineered lentiviral vector of any of claims 63-83, wherein the NiV-F protein or biologically active portion thereof is a truncated NiV-F that is truncated by or by at least 22 amino acids or by or by at least 20 amino acids at or near the C-terminus of wild-type NiV-F set forth in SEQ ID NO:30.

85. The engineered lentiviral vector of any of claims 63-84, wherein the NiV-F protein or biologically active portion thereof has a 22 amino acid truncation at or near the C-terminus of the wild-type NiV-F protein set forth in SEQ ID NO:30.

86. The engineered lentiviral vector of any of claims 63-85, wherein the NiV-F protein or biologically active portion thereof comprises a deletion in its cytoplasmic tail and lacks amino acid residues 525-546 of SEQ ID NO:30.

87. The engineered lentiviral vector of any of claims 63-86, wherein the NiV-F protein or biologically active portion thereof has the sequence set forth in SEQ ID NO: 16 or a sequence of amino acids that exhibits at least at or about 80%, 85%, 90%, or 95% sequence identity to the sequence set forth in SEQ ID NO: 16.

88. The engineered lentiviral vector of any of claims 63-87, wherein the NiV-F protein or biologically active portion thereof is set forth in SEQ ID NO:16.

89. The engineered lentiviral vector of any of claims 63-86, wherein the NiV-F protein or biologically active portion thereof has the amino acid sequence set forth in SEQ ID NO:21 or a sequence of amino acids that exhibits at least at or about 80%, 85%, 90%, or 95% sequence identity to the sequence set forth in SEQ ID NO:21.

90. The engineered lentiviral vector of any of claims 63-86 and 89, wherein the NiV-F protein or biologically active portion thereof is set forth in SEQ ID NO:21.

91. The engineered lentiviral vector of any of claims 63-77, 81-82, and 89-90, wherein the NiV-G protein or biologically active portion thereof has the sequence set forth in SEQ ID NO:18, and the NiV-F protein or biologically active portion thereof has the sequence set forth in SEQ ID NO:21.

92. The engineered lentiviral vector of any of claims 66-91, wherein the targeting agent is an antibody or antibody fragment, a Design ankyrin repeat protein (DARPin), or an antigen-binding fibronectin type III (Fn3) scaffold that binds to the target molecule.

93. The engineered lentiviral vector of any of claims 66-92, wherein the targeting agent is a single domain antibody that binds to the target molecule.

94. The engineered lentiviral vector of any of claims 66-93, wherein the targeting agent is a VHH that binds to the target molecule.

95. The engineered lentiviral vector of any of claims 66-93, wherein the targeting agent is a single chain variable fragment (scFv) that binds to the target molecule.

96. The engineered lentiviral vector of any of claims 66-95, wherein the target molecule is expressed on a target cell.

97. The engineered lentiviral vector of claim 96, wherein the target cell comprises of a B cell, a T cell, a natural killer cell, an islet cell, a glial progenitor cell, a cardiac cell, a blood cell, a hepatocyte, a stem cell, or an induced pluripotent stem cell; and a target antigen is present on the surface of the target cell.

98. The engineered lentiviral vector of claim 96 or 97, wherein the target cell is a T cell, B cell, natural killer cell, macrophage, or monocyte.

99. The engineered lentiviral vector of claim 98, wherein the target cell is a T cell.

100. The engineered lentiviral vector of claim 98 or 99, wherein the T cell is a CD3+ T cell, a CD4+ T cell, a CDS+ T cell, a naive T cell, a regulatory T (Treg) cell, a non-regulatory T cell, a Th1 cell, a Th2 cell, a Th9 cell, a Th17 cell, a T-follicular helper (Tfh) cell, a cytotoxic T lymphocyte (CTL), an effector T (Teff) cell, a central memory T cell, an effector memory T cell, an effector memory T cell expressing CD45RA (TEMRA cell), a tissue-resident memory (Trm) cell, a virtual memory T cell, an innate memory T cell, a memory stem cell (Tse), or a γδ T cell.

101. The engineered lentiviral vector of any one of claims 98-100, wherein the T cell is a cytotoxic T cell, a helper T cell, a memory T cell, a regulatory T cell, or a tumor infiltrating lymphocyte.

102. The engineered lentiviral vector of any one of claims 98-101, wherein the T cell is a human T cell.

103. The engineered lentiviral vector of any one of claims 98-102, wherein the T cell is an autologous T cell.

104. The engineered lentiviral vector of any one of claims 98-102, wherein the T cell is an allogeneic T cell.

105. The engineered lentiviral vector of claim 103 or 104, wherein the autologous T cell or the allogeneic T cell is a primary T cell.

106. The engineered lentiviral vector of any one of claims 97-104, wherein the target cell has been differentiated from an embryonic stem cell (ESC) or an induced pluripotent stem cell (iPSC).

107. The engineered lentiviral vector of any one of claims 92-106, wherein the targeting agent targets CD8, optionally, the targeting agent is an scFv that targets CD8.

108. The engineered lentiviral vector of any one of claims 92-107, wherein the targeting agent targets CD4, optionally, the targeting agent is an scFv that targets CD4.

109. The engineered lentiviral vector of any one of claims 1-108, wherein the first genome further comprises one or more of: a promoter, an RSV promoter, a T7 promoter, a UbC promoter, a 5′ LTR, a truncated HIV-1 5′ LTR, an HIV-1 Ψ packaging signal, a RRE sequence, HIV-1 cPPT/CTS sequence, a P2A sequence, a T2A sequence, a WPRE sequence, a 3′ LTR, a self-inactivating HIV-1 3′ LTR, a poly A sequence, an origin of replication (ori), SV40 ori, and a reporter gene.

110. The engineered lentiviral vector of any one of claims 1-109, wherein the second genome further comprises one or more of: a promoter, an RSV promoter, a T7 promoter, a UbC promoter, a 5′ LTR, a truncated HIV-1 5′ LTR, an HIV-1 Ψ packaging signal, a RRE sequence, HIV-1 cPPT/CTS sequence, a P2A sequence, a T2A sequence, a WPRE sequence, a 3′ LTR, a self-inactivating HIV-1 3′ LTR, a poly A sequence, an origin of replication (ori), SV40 ori, and a reporter gene.

111. An engineered lentiviral vector system comprising:
(i) one or more envelope plasmids,
(ii) a packaging plasmid, and
(iii) two or more transfer plasmids, wherein the two or more transfer plasmids comprise a first transfer plasmid that comprises a first transgene and a second transfer plasmid that comprises a second transgene, and wherein the first and the second transgene are different.

112. The engineered lentiviral vector system of claim 111, wherein the first transfer plasmid and the second transfer plasmid are present at a ratio of between about 1:1 and 1:10 or between about 1:10 and 1:1.

113. The engineered lentiviral vector system of claim 111 or 112, wherein the ratio of the first transfer plasmid and second transfer plasmid is about 1:1, about 1:2, about 1:3, about 1:4, about 1:5, about 1:6, about 1:7, about 1:8, about 1:9, about 1:10, about 2:1, about 3:1, about 4:1, about 5:1, about 6:1, about 7:1, about 8:1, about 9:1, about 10:1, about 2:3, about 3:2, about 2:5, about 3:5, about 5:2, or about 5:3.

114. The engineered lentiviral vector system of any one of claims 111-113, wherein the first transgene encodes a first CAR and the second transgene encodes a second CAR, wherein the first CAR and the second CAR comprise different antigen binding domains that target different antigens expressed on a target cell.

115. The engineered lentiviral vector system of claim 114, wherein:
a. the first CAR is a CD19 CAR and the second CAR is a CD22 CAR;
b. the first CAR is a B cell maturation agent (BCMA) CAR and the second CAR is a G-protein coupled receptor family C group 5 member D (GPRC5D) CAR;
c. the first CAR is a CD19 CAR and the second CAR is a CD20 CAR;
d. the first CAR is a CD20 CAR and the second CAR is a CD22 CAR; or
d. the first CAR is a CD19 CAR and the second CAR is a BCMA CAR.

116. The engineered lentiviral vector system of claim 114, wherein the first CAR and the second CAR are selected from the group comprising or consisting of a CD19 CAR, a CD22 CAR, B cell maturation agent (BCMA) CAR, a G-protein coupled receptor family C group 5 member D (GPRC5D) CAR, a CD5 CAR, a CD20 CAR, a CD23 CAR, a CD30 CAR, a CD33 CAR, CD38 CAR, a CD70 CAR, a CD123 CAR, a CD138 CAR, a Kappa CAR, Lambda CAR, a CD123 CAR, a LeY CAR, a NKG2D ligand CAR, a WT1 CAR, a GD2 CAR, a HER2 CAR, an EGFR CAR, an EGFRvIII CAR, a B7H3 CAR, a PSMA CAR, a PSCA CAR, a CAIX CAR, a CD171 CAR, a CEA CAR, a CSPG4 CAR, a EPHA2 CAR, a FAP CAR, a FRα CAR, a IL-13Rα CAR, a Mesothelin CAR, a MUC1 CAR, a MUC16 CAR, a ROR1 CAR, a C-Met CAR, a CD133 CAR, a Ep-CAM CAR, a GPC3 CAR, a HPV16-E6 CAR, a IL13Ra2 CAR, a MAGEA3 CAR, a MAGEA4 CAR, a MART1 CAR, a NY-ESO-1 CAR, a VEGFR2 CAR, a α-Folate receptor CAR, a CD24 CAR, a CD44v7/8 CAR, a EGP-2 CAR, a EGP-40 CAR, a erb-B2 CAR, a erb-B 2,3,4 CAR, a FBP CAR, a Fetal acethylcholine e receptor CAR, a GD2 CAR, a GD3 CAR, a HMW-MAA CAR, a IL-11Rα CAR, a KDR CAR, a Lewis Y CAR, a L1-cell adhesion molecule CAR, a MAGE-A1 CAR, a Oncofetal antigen (h5T4) CAR, a TAG-72 CAR, or a CD19/CD22 bispecific CAR.

117. The engineered lentiviral vector system of claim 114, the first CAR and the second CAR are selected from the group comprising or consisting of a CD19 CAR, a CD22 CAR, BCMA CAR, a GPRC5D CAR, a CD20 CAR, a CD19/CD22-bispecific CAR, a CD38 CAR, a CD123 CAR, or a CD138 CAR.

118. The engineered lentiviral vector system of claim 111-113, wherein the first transgene encodes a sequence-specific nuclease.

119. The engineered lentiviral vector system of claim 118, wherein the sequence-specific nuclease is an RNA-guided nuclease, a transcription activator-like effector nuclease (TALEN), a meganuclease, a CRISPR-associated transposase, and a TnpB polypeptide, or a zinc-finger nuclease (ZFN).

120. The engineered lentiviral vector system of claim 118 or 119, wherein the sequence-specific nuclease is selected from the group consisting of: Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, a CRISPR-associated transposase, and a TnpB polypeptide.

121. The engineered lentiviral vector system of claim 119, wherein the RNA-guided nuclease comprises a Cas nuclease.

122. The engineered lentiviral vector system of claim 121, wherein the Cas nuclease is a Type II or a Type V Cas protein.

123. The engineered lentiviral vector system of claim 121 or 122, wherein the Cas nuclease is selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, and Mad7.

124. The engineered lentiviral vector system of claim 121 or 123, wherein the Cas nuclease a Cas9 or a Cas12b.

125. The engineered lentiviral vector system of any one of claims 121-124, wherein the RNA-guided nuclease comprises a Cas nuclease and a guide RNA (CRISPR-Cas combination).

126. The engineered lentiviral vector system of claim 111-113, wherein the first transgene encodes a gRNA.

127. The engineered lentiviral vector system of claim 111-113, wherein the first transgene encodes an antibody or portion thereof.

128. The engineered lentiviral vector system of claim 111-113, wherein the first transgene encodes an antigen.

129. The engineered lentiviral vector system of claim 111-113, wherein the first transgene encodes a therapeutic polypeptide.

130. The engineered lentiviral vector system of claim 129, wherein the therapeutic polypeptide is useful for protein replacement therapy.

131. The engineered lentiviral vector system of claim 111-113, wherein the first transgene encodes a CAR.

132. The engineered lentiviral vector system any one of claims 111-113 and 118-131, wherein the second transgene encodes a sequence-specific nuclease.

133. The engineered lentiviral vector system of claim 132, wherein the sequence-specific nuclease is an RNA-guided nuclease, a transcription activator-like effector nuclease (TALEN), a meganuclease, a CRISPR-associated transposase, and a TnpB polypeptide, or a zinc-finger nuclease (ZFN).

134. The engineered lentiviral vector system of claim 132 or 133, wherein the sequence-specific nuclease is selected from the group consisting of: Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, a CRISPR-associated transposase, and a TnpB polypeptide.

135. The engineered lentiviral vector system of claim 133, wherein the RNA-guided nuclease comprises a Cas nuclease.

136. The engineered lentiviral vector system of claim 135, wherein the Cas nuclease is a Type II or Type V Cas protein.

137. The engineered lentiviral vector system of claim 135 or 136, wherein the Cas nuclease is selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, and Mad7.

138. The engineered lentiviral vector system of claim 135 or 136, wherein the Cas nuclease a Cas9 or a Cas12b.

139. The engineered lentiviral vector system of any one of claims 133-138, wherein the RNA-guided nuclease comprises a Cas nuclease and a guide RNA (CRISPR-Cas combination).

140. The engineered lentiviral vector system of any one of claims 111-113 and 118-131, wherein the second transgene encodes a gRNA.

141. The engineered lentiviral vector system of any one of claims 111-113 and 118-131, wherein the second transgene encodes an antibody or portion thereof.

142. The engineered lentiviral vector system of any one of claims 111-113 and 118-131, wherein the second transgene encodes an antigen.

143. The engineered lentiviral vector system of any one of claims 111-113 and 118-131, wherein the second transgene encodes a therapeutic polypeptide.

144. The engineered lentiviral vector system of claim 143, wherein the therapeutic polypeptide is useful for protein replacement therapy.

145. The engineered lentiviral vector system of any one of any one of claims 111-113 and 118-131, wherein the second transgene encodes a CAR.

146. The engineered lentiviral vector system of any one of claims 111-145, wherein the first transfer plasmid and/or the second transfer plasmid further comprises a transgene encoding a tolerogenic factor.

147. The engineered lentiviral vector system of any one of claims 111-146, wherein the first transgene and/or the second transgene comprises a biscistronic or a multicistronic expression cassette.

148. The engineered lentiviral vector system of claim 146 or 147, wherein the tolerogenic factor is selected from the group comprising or consisting of CD47, a SIRPα engager, A20/TNFAIP3, B2M-HLA-E, CD16, CD16 Fc receptor, CD24, CD27, CD35, CD39, CD46, CD47, CD52, CD55, CD59, CD64, CD200, CCL21, CCL22, CTLA4-Ig, C1 inhibitor, CR1, DUX4, FASL, HLA-C, HLA-E, HLA-E heavy chain, HLA-F, HLA-G, H2-M3, IDO1, IL-10, IL15-RF, IL-35, IL-39, MANF, Mfge8, PD-L1, or Serpinb9.

149. The engineered lentiviral vector system of any one of claims 146-148, wherein the tolerogenic factor is CD47.

150. The engineered lentiviral vector system of any one of claims 146-148, wherein the first transfer plasmid comprises a first tolerogenic factor and the second transfer plasmid comprises a second tolerogenic factor, and the first and the second tolerogenic factors are different.

151. The engineered lentiviral vector system of claim 150, wherein the first tolerogenic factor and the second tolerogenic factor are selected from the group comprising or consisting of CD47, a SIRPα engager, A20/TNFAIP3, B2M-HLA-E, CD16, CD16 Fc receptor, CD24, CD27, CD35, CD39, CD46, CD47, CD52, CD55, CD59, CD64, CD200, CCL21, CCL22, CTLA4-Ig, C1 inhibitor, CR1, DUX4, FASL, HLA-C, HLA-E, HLA-E heavy chain, HLA-F, HLA-G, H2-M3, IDO1, IL-10, IL15-RF, IL-35, IL-39, MANF, Mfge8, PD-L1, or Serpinb9.

152. The engineered lentiviral vector system of any one of claims 146-149, wherein first transfer plasmid comprises a tolerogenic factor and the second transfer plasmid does not comprise a tolerogenic factor.

153. The engineered lentiviral vector system any one of claims 111-145, wherein the first transfer plasmid further comprises a transgene encoding CD47 and the second plasmid further comprises a transgene encoding CD47.

154. The engineered lentiviral vector system of any one of claims 111-153, the one or more envelope plasmids comprise a sequence encoding one or more fusogens.

155. The engineered lentiviral vector system of claim 154, wherein the one or more fusogens comprise at least one fusogen that has a tropism for B cells, T cells, natural killer cells, islet cells, glial progenitor cells, neuronal cells, hematopoietic stem cells, cardiac cells, hepatocytes, stem cells, or induced pluripotent stem cells.

156. The engineered lentiviral vector system of any one of claims 154-155, wherein the one or more fusogens comprise at least one fusogen that has an endogenous tropism.

157. The engineered lentiviral vector system of any one of claims 154-156, wherein the one or more fusogens comprise at least one fusogen that has engineered tropism.

158. The engineered lentiviral vector system of any one of claims 154-157, wherein the one or more fusogens comprise one or more viral fusogens.

159. The engineered lentiviral vector system of any one of claims 154-158, wherein the one or more fusogens comprise at least one fusogen that is involved in attachment of a viral vector to a cell membrane.

160. The engineered lentiviral vector system of any one of claims 154-159, wherein the one or more fusogens comprise at least one fusogen that is involved in directing fusion of the lipid bilayer of a viral vector and a cell membrane.

161. The engineered lentiviral vector system of any one of claims 154-160, wherein the one or more fusogens comprise one or more paramyxovirus envelope proteins or biologically active portions thereof.

162. The engineered lentiviral vector system of claim 161, wherein the one or more paramyxovirus envelope proteins or biologically active portions thereof comprises a paramyxovirus glycoprotein (Protein G) or biologically active portion thereof.

163. The engineered lentiviral vector system of claim 161 or 162, wherein the one or more paramyxovirus envelope proteins or biologically active portions thereof comprises a paramyxovirus fusion protein (Protein F) or biologically active portion thereof.

164. The engineered lentiviral vector system of any one of claims 154-163, wherein the one or more fusogens comprise a Nipah virus fusion protein or a functional variant thereof.

165. The engineered lentiviral vector system of any one of claims 154-164, wherein the one or more fusogens comprise a Nipah virus F glycoprotein (NiV-F) or a biologically active portion thereof and a Nipah virus G glycoprotein (NiV-G) or a biologically active portion thereof.

166. The engineered lentiviral vector system any one of claims 154-165, wherein the one or more fusogens comprise one or more chimeric proteins.

167. The engineered lentiviral vector system of claim 166, wherein the one or more chimeric proteins comprise at least one chimeric protein that comprises a paramyxovirus envelope protein or biologically active portion thereof.

168. The engineered lentiviral vector system of claim 166 or 167, wherein the one or more chimeric proteins comprise at least one chimeric protein that comprises a targeting agent that targets a target molecule.

169. The engineered lentiviral vector system of any one of claims 166-168, wherein the one or more chimeric proteins comprise at least one chimeric protein that comprises (i) a paramyxovirus envelope protein or biologically active portion thereof and (ii) an targeting agent.

170. The engineered lentiviral vector system of any one of claims 165-169, wherein the NiV-G protein or biologically active portion thereof is fused to a targeting agent.

171. The engineered lentiviral vector system of claim 170, wherein the targeting agent is fused to the C-terminus of the NiV-G protein or biologically active portion thereof.

172. The engineered lentiviral vector system of any of claims 165-171, wherein the NiV-G or biologically active portion thereof is a wild-type NiV-G protein or a functionally active variant or biologically active portion thereof.

173. The engineered lentiviral vector system of any of claims 165-171, wherein the NiV-G protein or biologically active portion thereof is truncated and lacks up to 40 contiguous amino acid residues at or near the N-terminus of the wild-type NiV-G protein set forth in SEQ ID NO:4.

174. The engineered lentiviral vector system of any of claims 165-171 and 173, wherein the NiV-G protein or biologically active portion thereof is a biologically active portion that is a truncated NiV-G that has a deletion of amino acids 2-34 at or near the N-terminus of wild-type NiV-G set forth in SEQ ID NO:4.

175. The engineered lentiviral vector system of any of claims 165-174, wherein the NiV-G protein or biologically active portion thereof does not include an initial methionine.

176. The engineered lentiviral vector system of any of claims 165-175, wherein the NiV-G protein or biologically active portion thereof has the amino acid sequence set forth in SEQ ID NO:42 or a sequence of amino acids that exhibits at least at or about 80%, 85%, 90%, or 95% sequence identity to the sequence set forth in SEQ ID NO:42.

177. The engineered lentiviral vector system of any of claims 165-176, wherein the NiV-G protein or biologically active portion thereof is set forth in SEQ ID NO:42.

178. The engineered lentiviral vector system of any of claims 154-177, wherein at least one of the one or more fusogens comprise one or more modifications to reduce binding to its native receptor.

179. The engineered lentiviral vector system of claim 178, wherein the NiV-G protein or biologically active portion thereof is a mutant NiV-G protein or biologically active portion thereof that exhibits reduced binding to Ephrin B2 or Ephrin B3.

180. The engineered lentiviral vector system of claim 178 or 179, wherein the NiV-G protein or biologically active portion thereof comprises one or more amino acid substitutions corresponding to amino acid substitutions selected from the group consisting of E501A, W504A, Q530A, and E533A with reference to numbering set forth in SEQ ID NO:4.

181. The engineered lentiviral vector system of any of claims 178-180, wherein the NiV-G protein or biologically active portion thereof has the amino acid sequence set forth in SEQ ID NO: 17 or a sequence of amino acids that exhibits at least at or about 80%, 85%, 90%, or 95% sequence identity to the sequence set forth in SEQ ID NO:17.

182. The engineered lentiviral vector system of any of claims 178-180, wherein the NiV-G protein or biologically active portion thereof is set forth in SEQ ID NO:17.

183. The engineered lentiviral vector system of any of claims 178-180, wherein the NiV-G protein or the biologically active portion has the amino acid sequence set forth in SEQ ID NO: 18 or a sequence of amino acids that exhibits at least at or about 80%, 85%, 90%, or 95% sequence identity to the sequence set forth in SEQ ID NO: 18.

184. The engineered lentiviral vector system of any of claims 178-180, wherein the NiV-G protein or biologically active portion thereof is set forth in SEQ ID NO:18.

185. The engineered lentiviral vector system of any of claims 165-184, wherein the NiV-F protein or the biologically active portion thereof is a wild-type NiV-F protein or a functionally active variant or biologically active portion thereof.

186. The engineered lentiviral vector system of any of claims 165-185, wherein the NiV-F protein or biologically active portion thereof is a truncated NiV-F that is truncated by or by at least 22 amino acids or by or by at least 20 amino acids at or near the C-terminus of wild-type NiV-F set forth in SEQ ID NO:30.

187. The engineered lentiviral vector system of any of claims 165-186, wherein the NiV-F protein or biologically active portion thereof has a 22 amino acid truncation at or near the C-terminus of the wild-type NiV-F protein set forth in SEQ ID NO:30.

188. The engineered lentiviral vector system of any of claims 165-187, wherein the NiV-F protein or biologically active portion thereof comprises a deletion in its cytoplasmic tail and lacks amino acid residues 525-546 of SEQ ID NO:30.

189. The engineered lentiviral vector system of any of claims 165-188, wherein the NiV-F protein or biologically active portion thereof has the sequence set forth in SEQ ID NO: 16 or a sequence of amino acids that exhibits at least at or about 80%, 85%, 90%, or 95% sequence identity to the sequence set forth in SEQ ID NO: 16.

190. The engineered lentiviral vector system of any of claims 165-189, wherein the NiV-F protein or biologically active portion thereof is set forth in SEQ ID NO:16.

191. The engineered lentiviral vector system of any of claims 165-188, wherein the NiV-F protein or biologically active portion thereof has the amino acid sequence set forth in SEQ ID NO:21 or a sequence of amino acids that exhibits at least at or about 80%, 85%, 90%, or 95% sequence identity to the sequence set forth in SEQ ID NO:21.

192. The engineered lentiviral vector system of any of claims 165-188 and 191, wherein the NiV-F protein or biologically active portion thereof is set forth in SEQ ID NO:21.

193. The engineered lentiviral vector system of any of claims 165-179, 183-184, and 191-192, wherein the NiV-G protein or biologically active portion thereof has the sequence set forth in SEQ ID NO:18, and the NiV-F protein or biologically active portion thereof has the sequence set forth in SEQ ID NO:21.

194. The engineered lentiviral vector system of any of claims 168-193, wherein the targeting agent is an antibody or antibody fragment, a Design ankyrin repeat protein (DARPin), or an antigen-binding fibronectin type III (Fn3) scaffold that binds to the target molecule.

195. The engineered lentiviral vector system of any of claims 168-194, wherein the targeting agent is a single domain antibody that binds to the target molecule.

196. The engineered lentiviral vector system of any of claims 168-195, wherein the targeting agent is a VHH that binds to the target molecule.

197. The engineered lentiviral vector system of any of claims 168-195, wherein the targeting agent is a single chain variable fragment (scFv) that binds to the target molecule.

198. The engineered lentiviral vector system of any of claims 168-197, wherein the target molecule is expressed on a target cell.

199. The engineered lentiviral vector system of claim 198, wherein the target cell comprises of a B cell, a T cell, a natural killer cell, an islet cell, a glial progenitor cell, a cardiac cell, a blood cell, a hepatocyte, a stem cell, or an induced pluripotent stem cell; and a target antigen is present on the surface of the target cell.

200. The engineered lentiviral vector system of claim 198 or 199, wherein the target cell is a T cell, B cell, natural killer cell, macrophage, or monocyte.

201. The engineered lentiviral vector system of claim 200, wherein the target cell is a T cell.

202. The engineered lentiviral vector system of claim 200 or 201, wherein the T cell is a CD3+ T cell, a CD4+ T cell, a CDS+ T cell, a naive T cell, a regulatory T (Treg) cell, a non-regulatory T cell, a Th1 cell, a Th2 cell, a Th9 cell, a Th17 cell, a T-follicular helper (Tfh) cell, a cytotoxic T lymphocyte (CTL), an effector T (Teff) cell, a central memory T cell, an effector memory T cell, an effector memory T cell expressing CD45RA (TEMRA cell), a tissue-resident memory (Trm) cell, a virtual memory T cell, an innate memory T cell, a memory stem cell (Tse), or a γδ T cell.

203. The engineered lentiviral vector system of any one of claims 200-202, wherein the T cell is a cytotoxic T cell, a helper T cell, a memory T cell, a regulatory T cell, or a tumor infiltrating lymphocyte.

204. The engineered lentiviral vector system of any one of claims 200-203, wherein the T cell is a human T cell.

205. The engineered lentiviral vector system of any one of claims 194-204, wherein the targeting agent targets CD8, optionally, the targeting agent is an scFv that targets CD8.

206. The engineered lentiviral vector system of any one of claims 194-205, wherein the targeting agent targets CD4, optionally, the targeting agent is an scFv that targets CD4.

207. A method of making engineered lentiviral vectors, comprising:
introducing an engineered lentiviral vector system of any one of claims 111-206 to producer cells, and
culturing the producer cells under conditions sufficient to produce the viral vectors.

208. The method of claim 207, wherein the producer cell is selected from the group consisting of CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells.

209. The method of claim 207 or 208, wherein the producer cell is a 293T cell.

210. The method of any one of claims 207-209, wherein the two or more transfer plasmids are present in different amounts.

211. The method of any one of claims 207-209, wherein the two or more transfer plasmids are present in the same amount.

212. The method of any one of claims 207-209, wherein the first transfer plasmid and the second transfer plasmid are present at a ratio of between about 1:1 and 1:10 or between about 1:10 and 1:1.

213. The method of any one of claims 207-212, wherein the ratio of the first transfer plasmid and second transfer plasmid is about 1:1, about 1:2, about 1:3, about 1:4, about 1:5, about 1:6, about 1:7, about 1:8, about 1:9, about 1:10, about 2:1, about 3:1, about 4:1, about 5:1, about 6:1, about 7:1, about 8:1, about 9:1, about 10:1, about 2:3, about 3:2, about 2:5, about 3:5, about 5:2, or about 5:3.

214. The method of any one of claims 207-213, wherein the first transgene encodes a first CAR and the second transgene encodes a second CAR, wherein the first CAR and the second CAR comprise different antigen binding domains that target different antigens expressed on a target cell.

215. The engineered lentiviral vector system of claim 214, wherein:
a. the first CAR is a CD19 CAR and the second CAR is a CD22 CAR;
b. the first CAR is a B cell maturation agent (BCMA) CAR and the second CAR is a G-protein coupled receptor family C group 5 member D (GPRC5D) CAR;
c. the first CAR is a CD19 CAR and the second CAR is a CD20 CAR;
d. the first CAR is a CD20 CAR and the second CAR is a CD22 CAR; or
d. the first CAR is a CD19 CAR and the second CAR is a BCMA CAR.

216. The engineered lentiviral vector system of claim 214, wherein the first CAR and the second CAR are selected from the group comprising or consisting of a CD19 CAR, a CD22 CAR, B cell maturation agent (BCMA) CAR, a G-protein coupled receptor family C group 5 member D (GPRC5D) CAR, a CD5 CAR, a CD20 CAR, a CD23 CAR, a CD30 CAR, a CD33 CAR, CD38 CAR, a CD70 CAR, a CD123 CAR, a CD138 CAR, a Kappa CAR, Lambda CAR, a CD123 CAR, a LeY CAR, a NKG2D ligand CAR, a WT1 CAR, a GD2 CAR, a HER2 CAR, an EGFR CAR, an EGFRvIII CAR, a B7H3 CAR, a PSMA CAR, a PSCA CAR, a CAIX CAR, a CD171 CAR, a CEA CAR, a CSPG4 CAR, a EPHA2 CAR, a FAP CAR, a FRα CAR, a IL-13Rα CAR, a Mesothelin CAR, a MUC1 CAR, a MUC16 CAR, a ROR1 CAR, a C-Met CAR, a CD133 CAR, a Ep-CAM CAR, a GPC3 CAR, a HPV16-E6 CAR, a IL13Ra2 CAR, a MAGEA3 CAR, a MAGEA4 CAR, a MART1 CAR, a NY-ESO-1 CAR, a VEGFR2 CAR, a α-Folate receptor CAR, a CD24 CAR, a CD44v7/8 CAR, a EGP-2 CAR, a EGP-40 CAR, a erb-B2 CAR, a erb-B 2,3,4 CAR, a FBP CAR, a Fetal acethylcholine e receptor CAR, a GD2 CAR, a GD3 CAR, a HMW-MAA CAR, a IL-11Rα CAR, a KDR CAR, a Lewis Y CAR, a L1-cell adhesion molecule CAR, a MAGE-A1 CAR, a Oncofetal antigen (h5T4) CAR, a TAG-72 CAR, or a CD19/CD22 bispecific CAR.

217. The engineered lentiviral vector system of claim 214, the first CAR and the second CAR are selected from the group comprising or consisting of a CD19 CAR, a CD22 CAR, BCMA CAR, a GPRC5D CAR, a CD20 CAR, a CD19/CD22-bispecific CAR, a CD38 CAR, a CD123 CAR, or a CD138 CAR.

218. A cell comprising an engineered lentiviral vector of any one of claims 1-110.

219. The cell of claim 218, wherein the first transgene and the second transgene are integrated into the genome of the cell.

220. The cell of claim 218 or 219, wherein the cell is a B cell, a T cell, a natural killer cell, an islet cell, a glial progenitor cell, a cardiac cell, a blood cell, a hepatocyte, a stem cell, or an induced pluripotent stem cell.

221. The cell of any one of claims 218-220, wherein the cell is a T cell, B cell, natural killer cell, macrophage, or monocyte.

222. The cell of claim 218 or 219, wherein the cell is a T cell.

223. The cell of claim 222, wherein the T cell is a CD3+ T cell, a CD4+ T cell, a CDS+ T cell, a naive T cell, a regulatory T (Treg) cell, a non-regulatory T cell, a Th1 cell, a Th2 cell, a Th9 cell, a Th17 cell, a T-follicular helper (Tfh) cell, a cytotoxic T lymphocyte (CTL), an effector T (Teff) cell, a central memory T cell, an effector memory T cell, an effector memory T cell expressing CD45RA (TEMRA cell), a tissue-resident memory (Trm) cell, a virtual memory T cell, an innate memory T cell, a memory stem cell (Tse), or a γδ T cell.

224. The cell of any one of claims 222-223, wherein the T cell is a cytotoxic T cell, a helper T cell, a memory T cell, a regulatory T cell, or a tumor infiltrating lymphocyte.

225. The cell of any one of claims 222-224, wherein the T cell is a human T cell.

226. The cell of any one of claims 222-225, wherein the T cell is an autologous T cell.

227. The engineered lentiviral vector of any one of claims 222-225, wherein the T cell is an allogeneic T cell.

228. The cell of claim 226 or 227, wherein the autologous T cell or the allogeneic T cell is a primary T cell.

229. The cell of any one of claims 218-227, wherein the cell has been differentiated from an embryonic stem cell (ESC) or an induced pluripotent stem cell (iPSC).

230. The cell of any one of claims 218-227, wherein the cell is a CAR-T cell expressing a first CAR and a second CAR.

231. The cell of claim 230, wherein:
a. the first CAR is a CD19 CAR and the second CAR is a CD22 CAR;
b. the first CAR is a B cell maturation agent (BCMA) CAR and the second CAR is a G-protein coupled receptor family C group 5 member D (GPRC5D) CAR;
c. the first CAR is a CD19 CAR and the second CAR is a CD20 CAR;
d. the first CAR is a CD20 CAR and the second CAR is a CD22 CAR; or
d. the first CAR is a CD19 CAR and the second CAR is a BCMA CAR.

232. A method of making a drug substance comprising an engineered lentiviral vector, comprising:
making engineered lentiviral vectors according to the method of any one of claims 207-217,
obtaining a subset of the producer cell culture comprising lentiviral vectors, and enriching lentiviral vectors from the subset of the producer cell culture.

233. The method of claim 232, further comprising removing producer cell DNA and/or producer cell protein from the subset of the producer cell culture.

234. The method of claim 232 or 233, further comprising adding a pharmaceutically acceptable excipient to the enriched lentiviral vectors.

235. A pharmaceutical composition comprising an engineered lentiviral vector of any one of claims 1-110, and a pharmaceutically acceptable carrier.

236. A method of making the cell of claims 215-231, comprising contacting a target cell with an engineered lentiviral vector of any of claims 1-110 or the pharmaceutical composition of claim 236.

237. A method of transducing a target cell, the method comprising contacting a target cell with an engineered lentiviral vector of any of claims 1-110 or the pharmaceutical composition of claim 235.

238. The method of claim 236 or 237, wherein the contacting is in vitro or ex vivo.

239. The method of claim 238, wherein the contacting is in vivo in a subject.

240. A method of treating a disease or condition in a subject comprising administering a cell of any one of claims 218-231 to a subject in need thereof.

241. The method of claim 240, wherein the cell is a CAR-T cell.

242. The method of claim 241, wherein the CAR-T cell expresses a first CAR and a second CAR, wherein:
a. the first CAR is a CD19 CAR and the second CAR is a CD22 CAR;
b. the first CAR is a B cell maturation agent (BCMA) CAR and the second CAR is a G-protein coupled receptor family C group 5 member D (GPRC5D) CAR;
c. the first CAR is a CD19 CAR and the second CAR is a CD20 CAR;
d. the first CAR is a CD20 CAR and the second CAR is a CD22 CAR; or
d. the first CAR is a CD19 CAR and the second CAR is a BCMA CAR.

243. The method of claim 242, wherein the first CAR and the second CAR are expressed by the cell at a ratio of between about 1:1 and 1:10 or between about 1:10 and 1:1.

244. The method claim 242 or 243, wherein the ratio of the first CAR and second CAR is about 1:1, about 1:2, about 1:3, about 1:4, about 1:5, about 1:6, about 1:7, about 1:8, about 1:9, about 1:10, about 2:1, about 3:1, about 4:1, about 5:1, about 6:1, about 7:1, about 8:1, about 9:1, about 10:1, about 2:3, about 3:2, about 2:5, about 3:5, about 5:2, or about 5:3.

245. The method of any of claims 240-244, wherein the disease or condition is a cancer.

246. The method of any of claims 240-245, wherein the disease or condition is a B cell acute lymphoblastic leukemia (B-ALL), diffuse large B-cell lymphoma, acute myeloid lymphoid leukemia, or multiple myeloma.

247. The method of claim 246, wherein relapse of the disease or condition is prevented or delayed compared to a subject diagnosed with relapse of the disease or condition that was treated with a CAR-T cell targeting a single antigenic target.

248. The method of claim 247, wherein the single antigenic target is CD19, CD20, CD22, or BCMA.

249. A method of treating a disease or condition in a subject comprising administering a viral vector of any of claims 1-110 or a pharmaceutical composition of claim 235 to a subject in need thereof.

250. The method of claim 249, wherein the disease or condition is a cancer.

251. The method of claim 250, wherein the cancer is a solid tumor, a lymphoma, or a leukemia.

252. The method of claim 250 or 251, wherein in the cancer is B cell acute lymphoblastic leukemia (B-ALL), diffuse large B-cell lymphoma, liver cancer, pancreatic cancer, breast cancer, ovarian cancer, colorectal cancer, lung cancer, non-small cell lung cancer, acute myeloid lymphoid leukemia, multiple myeloma, gastric cancer, gastric adenocarcinoma, pancreatic adenocarcinoma, glioblastoma, neuroblastoma, lung squamous cell carcinoma, hepatocellular carcinoma, and bladder cancer.
